(19) United States
(12) Patent Application Publication
(10) Pub. No.: US 2022/0085300 A1
(43) Pub. Date: Mar. 17, 2022

(54) ORGANIC LIGHT EMITTING DEVICE

(71) Applicant: LG CHEM, LTD., Seoul (KR)

(72) Inventors: Minjun Kim, Daejeon (KR); Young Seok Kim, Daejeon (KR); Seoyeon Kim, Daejeon (KR); Da Jung Lee, Daejeon (KR); Dong Hoon Lee, Daejeon (KR); Yongbum Cha, Daejeon (KR); Sang Duk Suh, Daejeon (KR); Donghee Kim, Daejeon (KR); Joongsuk Oh, Daejeon (KR); Seung Won Choi, Daejeon (KR); Jaehoon Sim, Daejeon (KR)

(21) Appl. No.: 17/431,613

(22) PCT Filed: Nov. 11, 2020
(86) PCT No.: PCT/KR2020/015815

§ 371 Date: Aug. 17, 2021

(30) Foreign Application Priority Data
Nov. 11, 2019 (KR) 10-2019-0143630
Nov. 11, 2020 (KR) 10-2020-0150222

(51) Int. Cl. H01L 51/00; C07D 405/04; C09K 11/06; C07D 405/14; C07D 409/14; C07D 405/10; C07C 211/54; C07D 307/91; C07D 333/76; C07D 209/86; C07C 211/61

(52) U.S. Cl. CPC H01L 51/0073; H01L 51/5012; C09K 11/06; H01L 51/0067; H01L 51/0058; H01L 51/0052; C07D 405/14; C07D 409/14; H01L 51/0074; H01L 51/0072; C07D 405/10; C07C 211/54; H01L 51/006; C07D 307/91; H01L 51/0061; C07D 333/76; C07D 209/86; C07C 211/61; C07D 405/04

(57) ABSTRACT

An organic light emitting device including a light emitting layer which comprises a compound represented by Chemical Formula 1 and a compound represented by Chemical Formula 2, and the organic light emitting device having improved driving voltage, efficiency and lifetime.

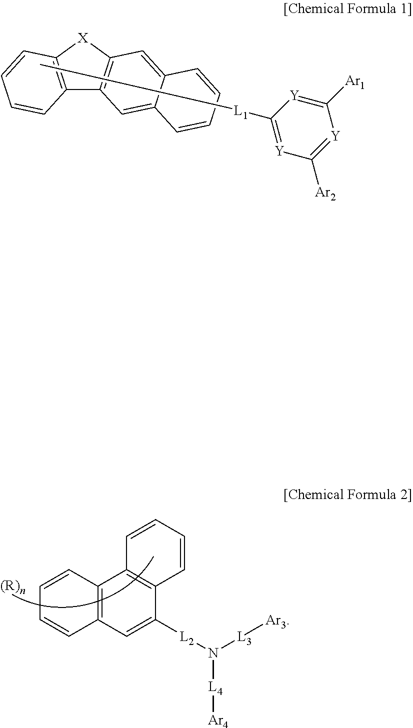

### CROSS-REFERENCE TO RELATED APPLICATION(S)

This application is a National Phase entry pursuant to 35 U.S.C § 371 of International Application No. PCT/KR2020/015815 filed on Nov. 11, 2020, and claims priority to and the benefit of Korean Patent Application No. 10-2019-0143630 filed on Nov. 11, 2019 and Korean Patent Application No. 10-2020-0150222 filed on Nov. 11, 2020, the disclosures of which are incorporated herein by reference in their entireties.

### FIELD OF DISCLOSURE

The present disclosure relates to relates to an organic light emitting device having improved driving voltage, efficiency and lifetime.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

In the organic light emitting devices as described above, there is a continuing need for the development of an organic light emitting device having improved driving voltage, efficiency and lifetime.

### RELATED ART

(Patent Literature 0001) Korean Unexamined Patent Publication No. 10-2000-0051826

### SUMMARY

The present disclosure relates to an organic light emitting device having improved driving voltage, efficiency and lifetime.

Provided herein is the following organic light emitting device:

An organic light emitting device including: an anode, a cathode, and a light emitting layer interposed between the anode and the cathode,

wherein the light emitting layer comprises a compound represented by the following Chemical Formula 1 and a compound represented by the following Chemical Formula 2.

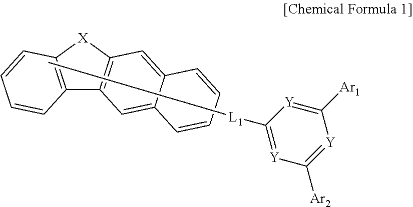

in Chemical Formula 1,

X is O or S,

each Y is independently N or CH, with the proviso that at least one of Y is N,

L₁ is a single bond; or a substituted or unsubstituted C₆₋₆₀ arylene,

Ar₁ and Ar₂ are each independently a substituted or unsubstituted C₆₋₆₀ aryl; or a substituted or unsubstituted C₂₋₆₀ heteroaryl containing any one or more selected from the group consisting of N, O and S,

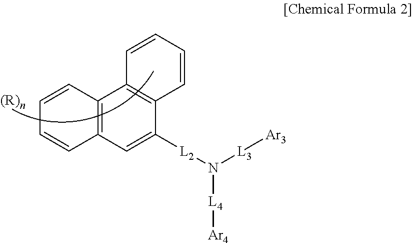

in Chemical Formula 2,

L₂ is a substituted or unsubstituted C₆₋₆₀ arylene,

L₃ and L₄ are each independently a single bond; or a substituted or unsubstituted C₆₋₆₀ arylene,

Ar₃ and Ar₄ are each independently a substituted or unsubstituted C₆₋₆₀ aryl; or a substituted or unsubstituted C₂₋₆₀ heteroaryl containing any one or more selected from the group consisting of N, O and S,

R is deuterium; or a substituted or unsubstituted C₆₋₆₀ aryl, and

n is an integer of 0 to 9.

### Advantageous Effects

The above-mentioned organic light emitting device has excellent driving voltage, efficiency and lifetime by containing the compound represented by Chemical Formula 1 and the compound represented by Chemical Formula 2 in the light emitting layer.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole transport layer 5, a light emitting layer 3, an electron transport layer 6, and a cathode 4.

### DETAILED DESCRIPTION

Hereinafter, embodiments of the present disclosure will be described in more detail to facilitate understanding of the invention.

As used herein, the notation

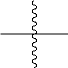

or [symbol] means a bond linked to another substituent group.

As used herein, the term “substituted or unsubstituted” means being unsubstituted or substituted with one or more substituents selected from the group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; or a heteroaryl containing at least one of N, O and S atoms, or being unsubstituted or substituted with a substituent to which two or more substituents of the above-exemplified substituents are connected. For example, “a substituent in which two or more substituents are connected” may be a biphenyl group. Namely, a biphenyl group may be an aryl group, or it may also be interpreted as a substituent in which two phenyl groups are connected.

In the present disclosure, the carbon number of a carbonyl group is not particularly limited, but is preferably 1 to 40. Specifically, the carbonyl group may be a compound having the following structural formulas, but is not limited thereto.

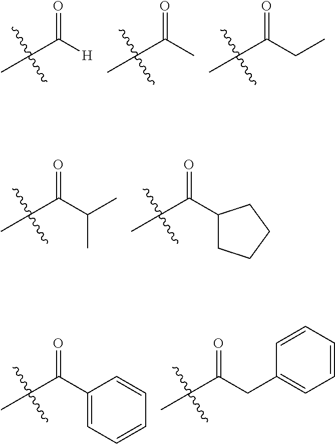

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a compound having the following structural formulas, but is not limited thereto.

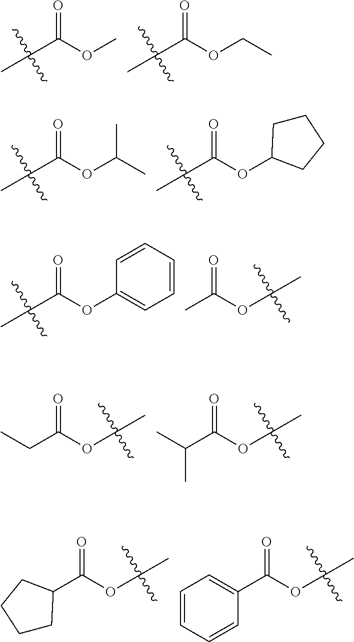

In the present disclosure, the carbon number of an imide group is not particularly limited, but is preferably 1 to 25. Specifically, the imide group may be a compound having the following structural formulas, but is not limited thereto.

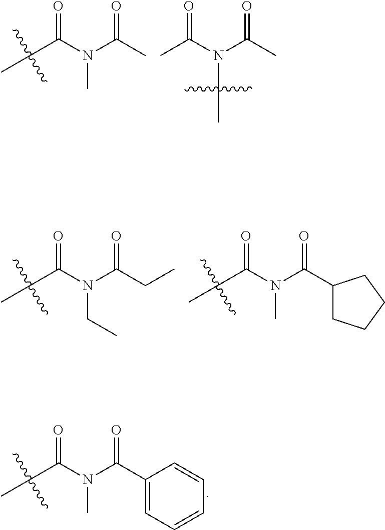

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohectylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dim ethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The aryl group may be a phenyl group, a biphenyl group, a terphenyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, or the like, but is not limited thereto.

In the present disclosure, the fluorenyl group may be substituted, and two substituents may be linked with each other to form a spiro structure. In the case where the fluorenyl group is substituted,

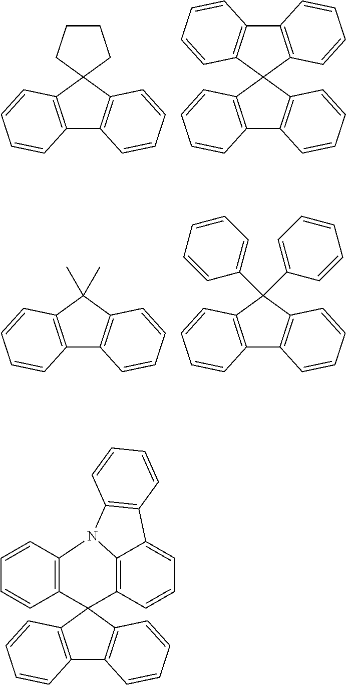

and the like can be formed. However, the structure is not limited thereto.

In the present disclosure, a heterocyclic group is a heterocyclic group containing one or more of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can be applied to the aforementioned description of the heterocyclic group. In the present disclosure, the alkenyl group in the aralkenyl group is the same as the aforementioned examples of the alkenyl group. In the present disclosure, the aforementioned description of the aryl group may be applied except that the arylene is a divalent group. In the present disclosure, the aforementioned description of the heteroaryl group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocyclic group is not a monovalent group but formed by combining two substituent groups.

Hereinafter, the present disclosure will be described in detail for each configuration.

Anode and Cathode

The anode and cathode used in the present disclosure mean electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SNO₂:Sb, conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Hole Injection Layer

The organic light emitting device according to the present disclosure may further include a hole injection layer on the anode, if necessary.

The hole injection layer is a layer injecting holes from an electrode, and the hole injection material is preferably a compound which has an ability of transporting the holes, a hole injection effect in the anode and an excellent hole injection effect to the light emitting layer or the light emitting material, prevents movement of an exciton generated in the light emitting layer to the electron injection layer or the electron injection material, and has an excellent thin film forming ability. It is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

Hole Transport Layer

The organic light emitting device according to the present disclosure may include a hole transport layer on the anode (or on a hole injection layer when the hole injection layer is present), if necessary.

The hole transport layer is a layer that receives holes from an anode or a hole injection layer and transports the holes to the light emitting layer. The hole transport material is suitably a material having large mobility to the holes, which may receive holes from the anode or the hole injection layer and transfer the holes to the light emitting layer.

Specific examples of the hole transport material include an arylamine-based organic material, a conductive polymer, a block copolymer in which a conjugate portion and a non-conjugate portion are present together, and the like, but are not limited thereto.

Light Emitting Layer

The light emitting layer used in the present disclosure means a layer that can emit light in the visible light region by combining holes and electrons transported from the anode and the cathode. Generally, the light emitting layer includes a host material and a dopant material, and in the present disclosure, the compound represented by Chemical Formula 1 and the compound represented by Chemical Formula 2 are included as a host

In Chemical Formula 1, preferably, each Y is N.

Preferably, L₁ is a single bond; phenylene; or naphthylene. More preferably, L₁ is a single bond;

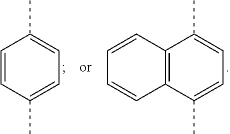

Preferably, Ar₁ and Ar₂ are each independently phenyl, biphenyl, terphenyl, naphthyl, phenanthrenyl, (phenyl)naphthyl, (naphthyl)phenyl, dimethylfluorenyl, diphenylfluorenyl, dibenzofuranyl, dibenzothiophenyl, carbazole-9-yl, 9-phenyl-9H-carbazolyl, each of which is independently unsubstituted or substituted with at least one deuterium. When Ar₁ or Ar₂ is substituted with at least one deuterium, each of them is preferably any one selected from the group consisting of the following:

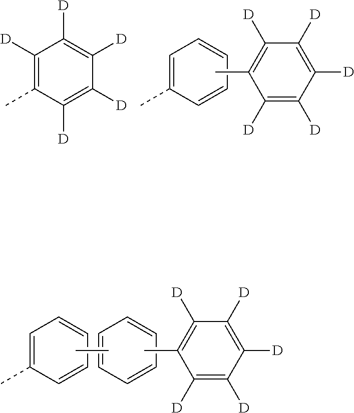

Preferably, Ar₁ is phenyl, biphenyl, or naphthyl, each of which is unsubstituted or substituted with at least one deuterium; and Ar₂ is phenyl, biphenyl, terphenyl, naphthyl, phenanthrenyl, (phenyl)naphthyl, (naphthyl)phenyl, dimethylfluorenyl, diphenylfluorenyl, dibenzofuranyl, dibenzothiophenyl, carbazol-9-yl, 9-phenyl-9H-carbazolyl, each of which is unsubstituted or substituted with at least one deuterium.

Representative examples of the compound represented by Chemical Formula 1 are as follows:

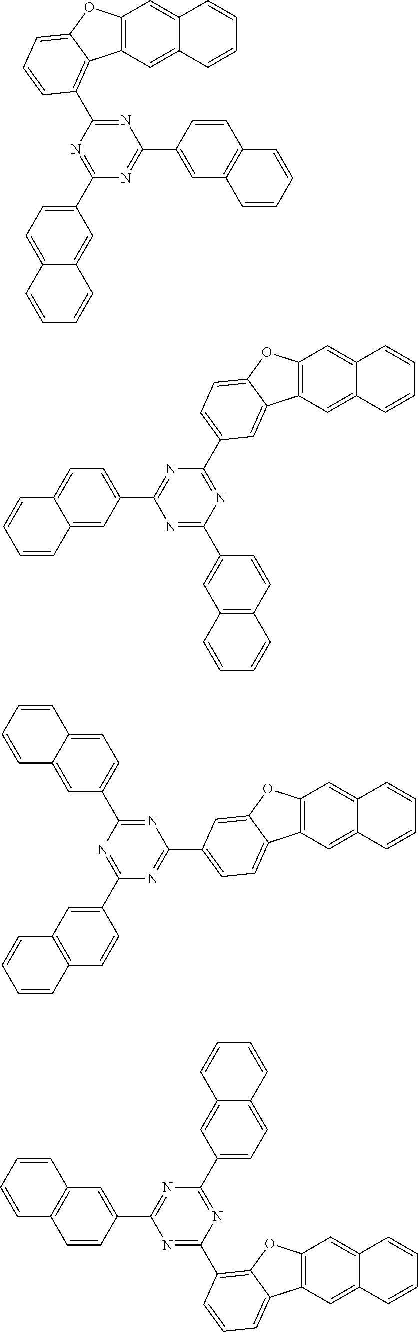 [SMILES: c1ccc2cc(-c3nc(-c4ccc5ccccc5c4)nc(-c4cccc5oc6cc7ccccc7cc6c45)n3)ccc2c1 ; c1ccc2cc(-c3nc(-c4ccc5ccccc5c4)nc(-c4ccc5oc6cc7ccccc7cc6c5c4)n3)ccc2c1 ; c1ccc2cc(-c3nc(-c4ccc5ccccc5c4)nc(-c4ccc5c(c4)oc4cc6ccccc6cc45)n3)ccc2c1 ; c1ccc2cc(-c3nc(-c4ccc5ccccc5c4)nc(-c4cccc5c4oc4cc6ccccc6cc45)n3)ccc2c1]

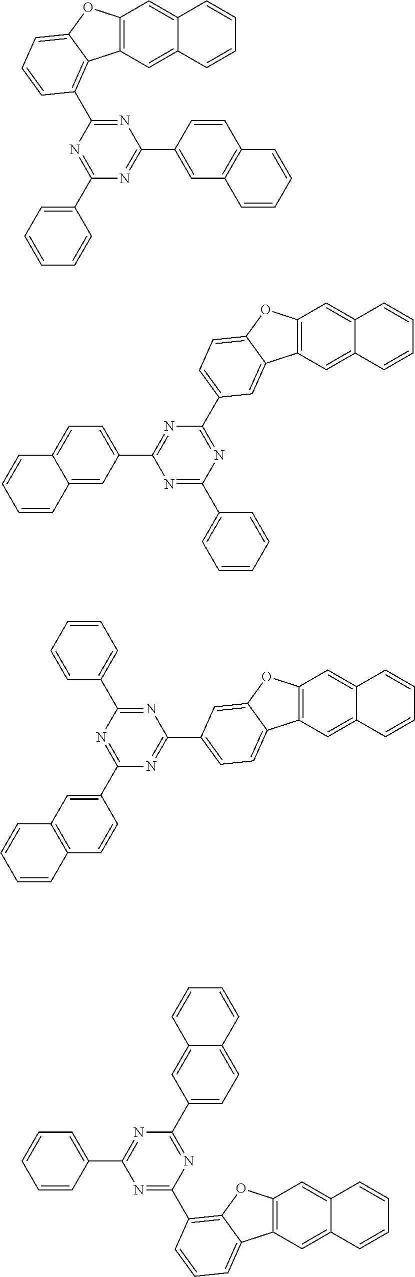 [SMILES: c1ccc(-c2nc(-c3ccc4ccccc4c3)nc(-c3cccc4oc5cc6ccccc6cc5c34)n2)cc1 ; c1ccc(-c2nc(-c3ccc4ccccc4c3)nc(-c3ccc4oc5cc6ccccc6cc5c4c3)n2)cc1 ; c1ccc(-c2nc(-c3ccc4ccccc4c3)nc(-c3ccc4c(c3)oc3cc5ccccc5cc34)n2)cc1 ; c1ccc(-c2nc(-c3ccc4ccccc4c3)nc(-c3cccc4c3oc3cc5ccccc5cc34)n2)cc1]

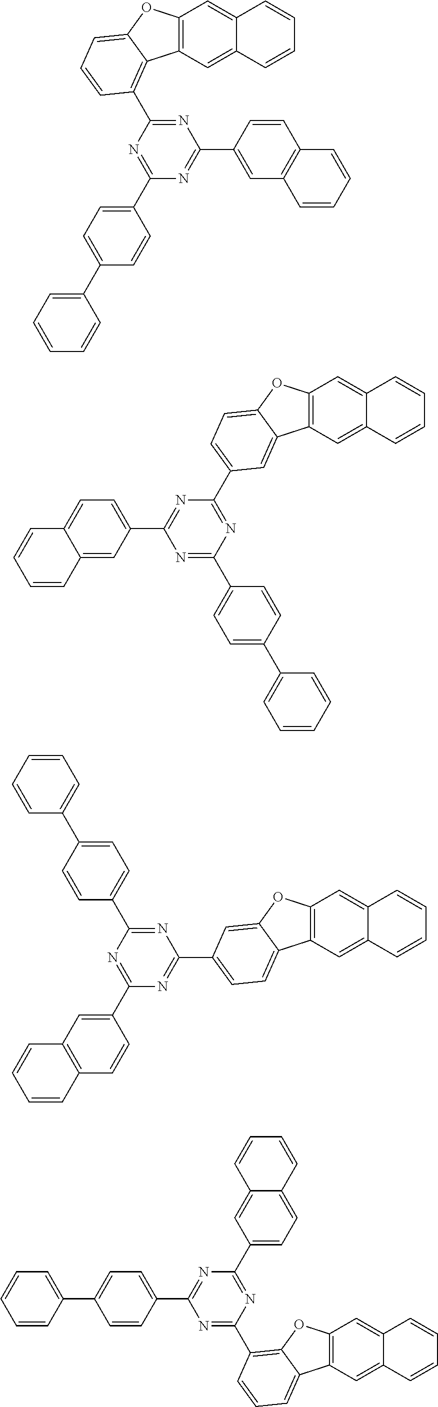 [SMILES: c1ccc(-c2ccc(-c3nc(-c4ccc5ccccc5c4)nc(-c4cccc5oc6cc7ccccc7cc6c45)n3)cc2)cc1 ; c1ccc(-c2ccc(-c3nc(-c4ccc5ccccc5c4)nc(-c4ccc5oc6cc7ccccc7cc6c5c4)n3)cc2)cc1 ; c1ccc(-c2ccc(-c3nc(-c4ccc5ccccc5c4)nc(-c4ccc5c(c4)oc4cc6ccccc6cc45)n3)cc2)cc1 ; c1ccc(-c2ccc(-c3nc(-c4ccc5ccccc5c4)nc(-c4cccc5c4oc4cc6ccccc6cc45)n3)cc2)cc1]

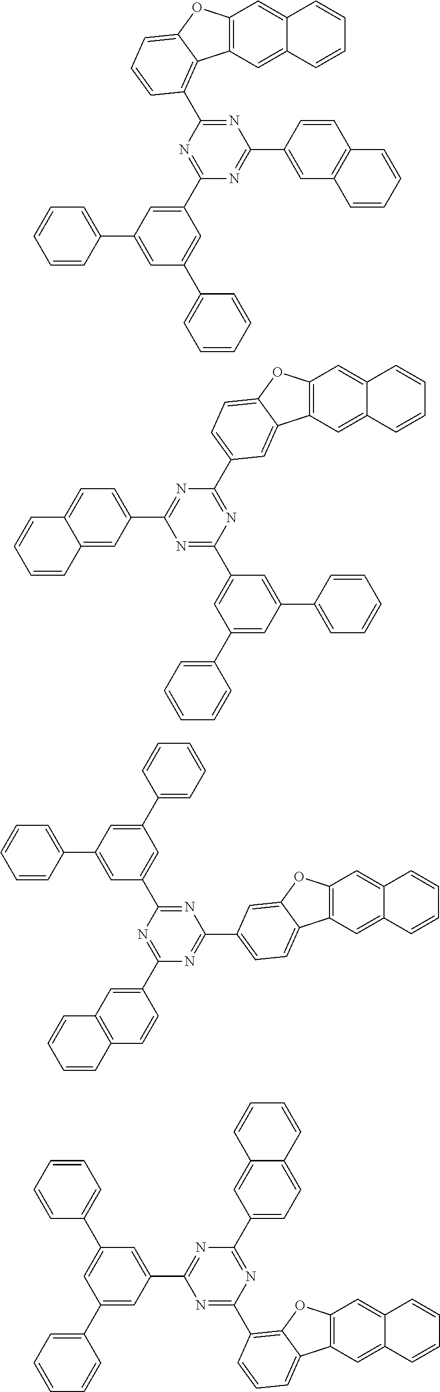

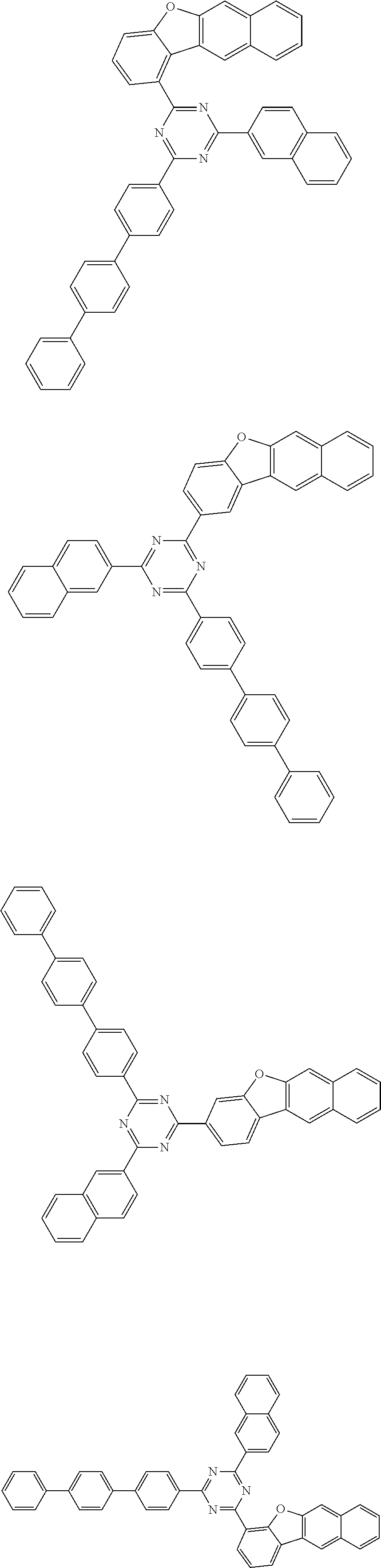 [SMILES: c1ccc(-c2ccc(-c3ccc(-c4nc(-c5ccc6ccccc6c5)nc(-c5cccc6oc7cc8ccccc8cc7c56)n4)cc3)cc2)cc1 ; c1ccc(-c2ccc(-c3ccc(-c4nc(-c5ccc6ccccc6c5)nc(-c5ccc6oc7cc8ccccc8cc7c6c5)n4)cc3)cc2)cc1 ; c1ccc(-c2ccc(-c3ccc(-c4nc(-c5ccc6ccccc6c5)nc(-c5ccc6c(c5)oc5cc7ccccc7cc56)n4)cc3)cc2)cc1 ; c1ccc(-c2ccc(-c3ccc(-c4nc(-c5ccc6ccccc6c5)nc(-c5cccc6c5oc5cc7ccccc7cc56)n4)cc3)cc2)cc1]

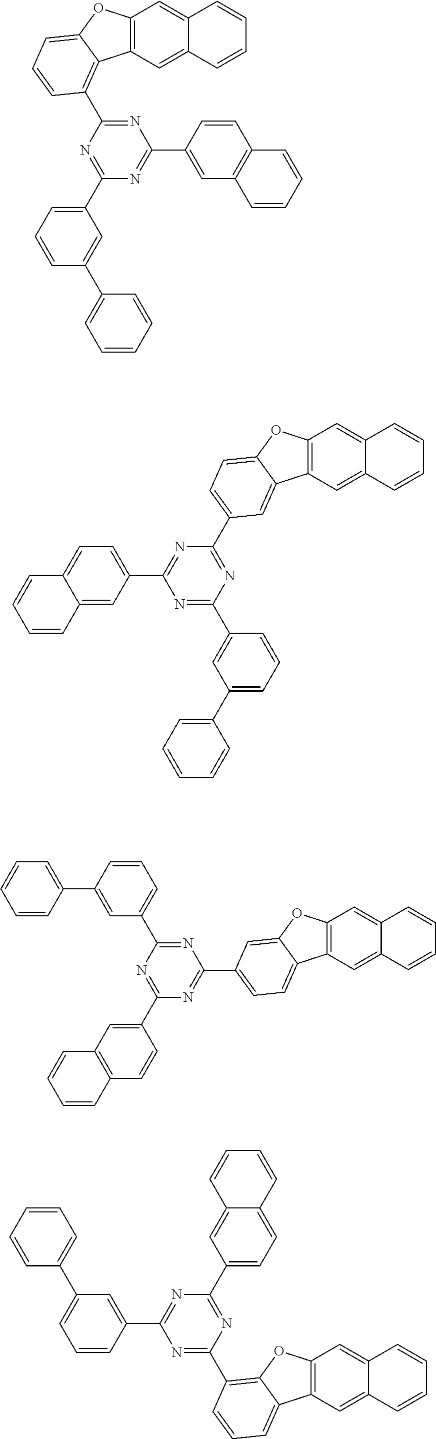 [SMILES: c1ccc(-c2cccc(-c3nc(-c4ccc5ccccc5c4)nc(-c4cccc5oc6cc7ccccc7cc6c45)n3)c2)cc1 ; c1ccc(-c2cccc(-c3nc(-c4ccc5ccccc5c4)nc(-c4ccc5oc6cc7ccccc7cc6c5c4)n3)c2)cc1 ; c1ccc(-c2cccc(-c3nc(-c4ccc5ccccc5c4)nc(-c4ccc5c(c4)oc4cc6ccccc6cc45)n3)c2)cc1 ; c1ccc(-c2cccc(-c3nc(-c4ccc5ccccc5c4)nc(-c4cccc5c4oc4cc6ccccc6cc45)n3)c2)cc1]

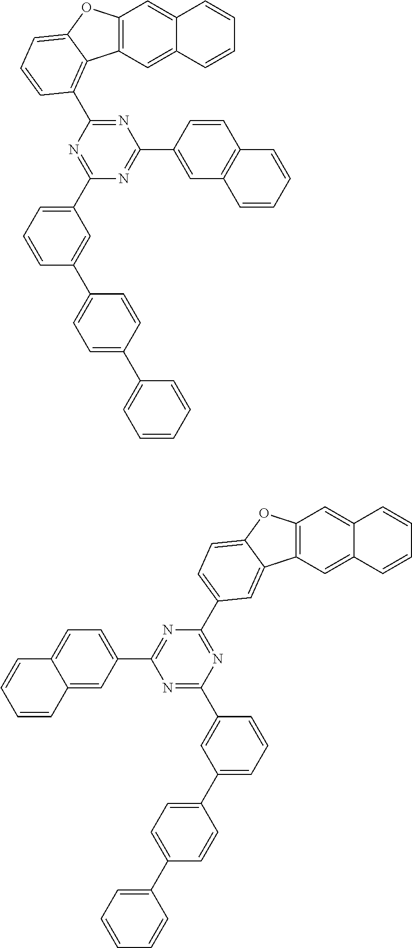 [SMILES: c1ccc(-c2ccc(-c3cccc(-c4nc(-c5ccc6ccccc6c5)nc(-c5cccc6oc7cc8ccccc8cc7c56)n4)c3)cc2)cc1 ; c1ccc(-c2ccc(-c3cccc(-c4nc(-c5ccc6ccccc6c5)nc(-c5ccc6oc7cc8ccccc8cc7c6c5)n4)c3)cc2)cc1]

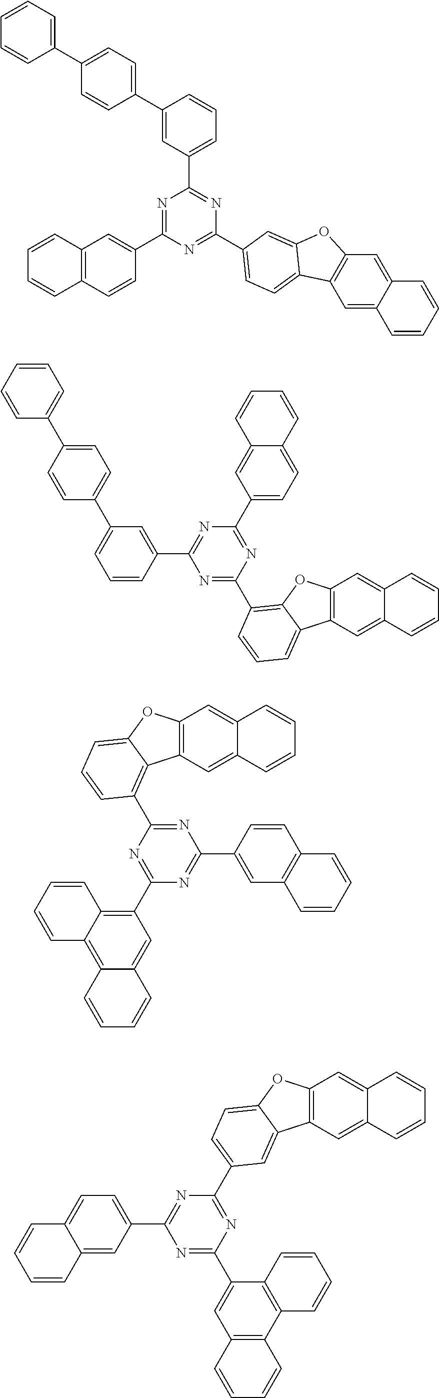 [SMILES: c1ccc(-c2ccc(-c3cccc(-c4nc(-c5ccc6ccccc6c5)nc(-c5ccc6c(c5)oc5cc7ccccc7cc56)n4)c3)cc2)cc1 ; c1ccc(-c2ccc(-c3cccc(-c4nc(-c5ccc6ccccc6c5)nc(-c5cccc6c5oc5cc7ccccc7cc56)n4)c3)cc2)cc1 ; c1ccc2cc(-c3nc(-c4cc5ccccc5c5ccccc45)nc(-c4cccc5oc6cc7ccccc7cc6c45)n3)ccc2c1 ; c1ccc2cc(-c3nc(-c4ccc5oc6cc7ccccc7cc6c5c4)nc(-c4cc5ccccc5c5ccccc45)n3)ccc2c1]

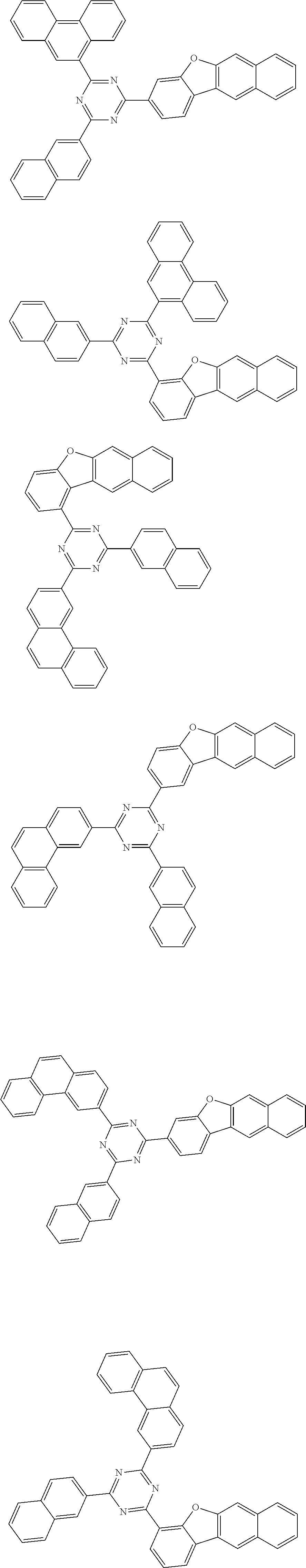

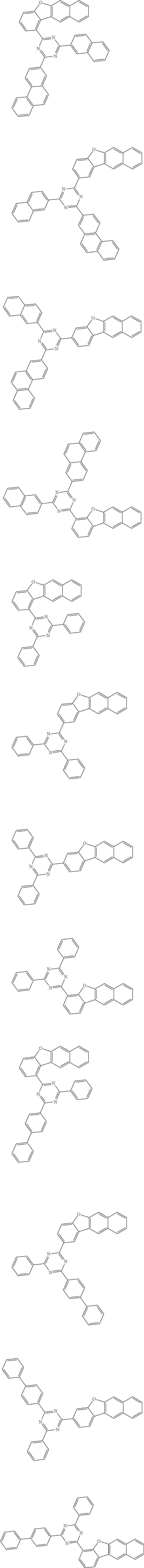

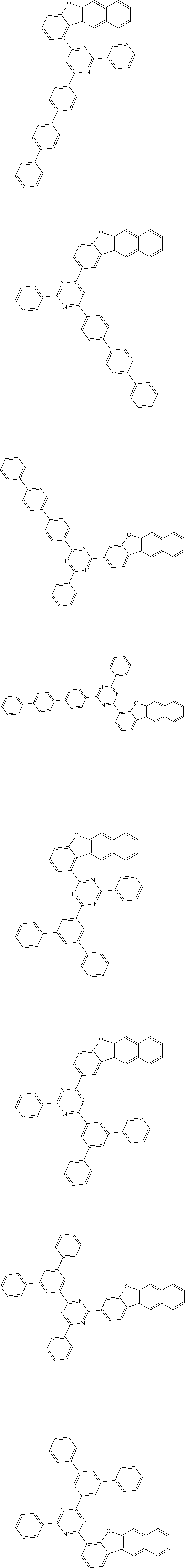

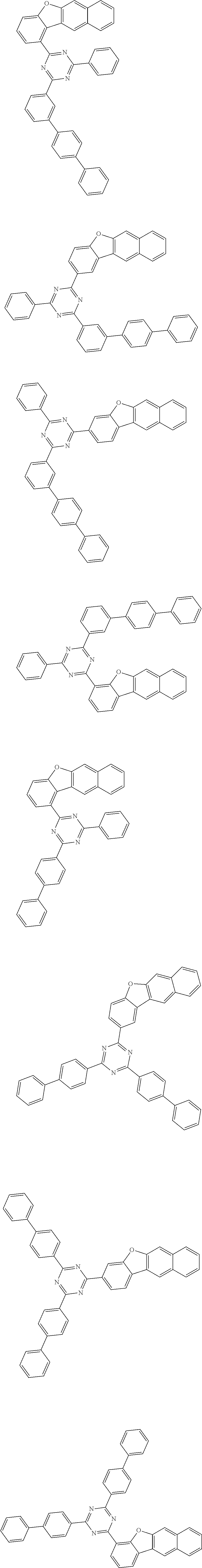

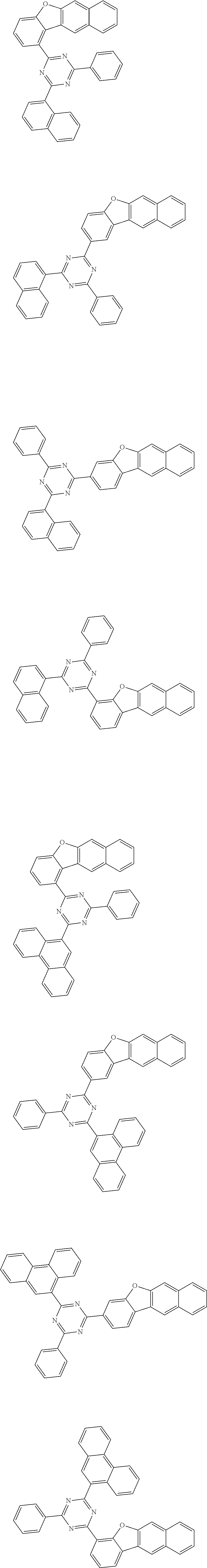

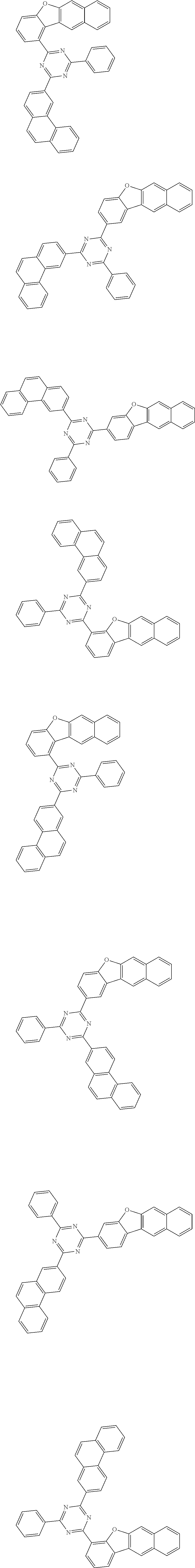

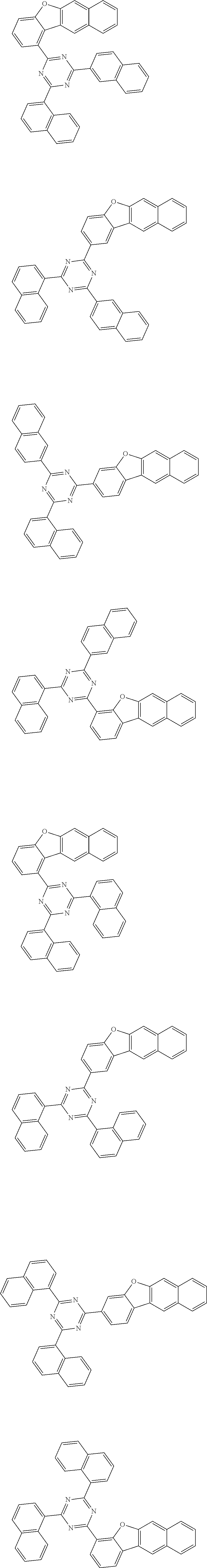

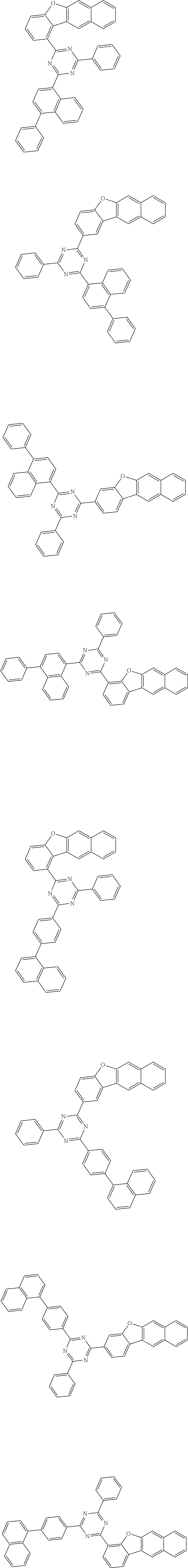

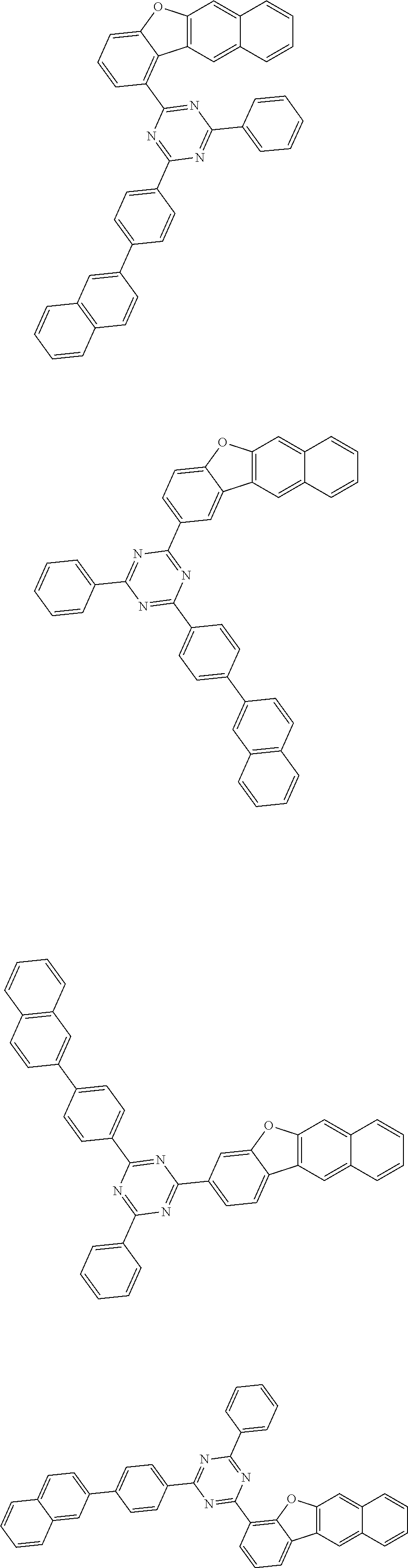 [SMILES: c1ccc(-c2nc(-c3ccc(-c4ccc5ccccc5c4)cc3)nc(-c3cccc4oc5cc6ccccc6cc5c34)n2)cc1 ; c1ccc(-c2nc(-c3ccc(-c4ccc5ccccc5c4)cc3)nc(-c3ccc4oc5cc6ccccc6cc5c4c3)n2)cc1 ; c1ccc(-c2nc(-c3ccc(-c4ccc5ccccc5c4)cc3)nc(-c3ccc4c(c3)oc3cc5ccccc5cc34)n2)cc1 ; c1ccc(-c2nc(-c3ccc(-c4ccc5ccccc5c4)cc3)nc(-c3cccc4c3oc3cc5ccccc5cc34)n2)cc1]

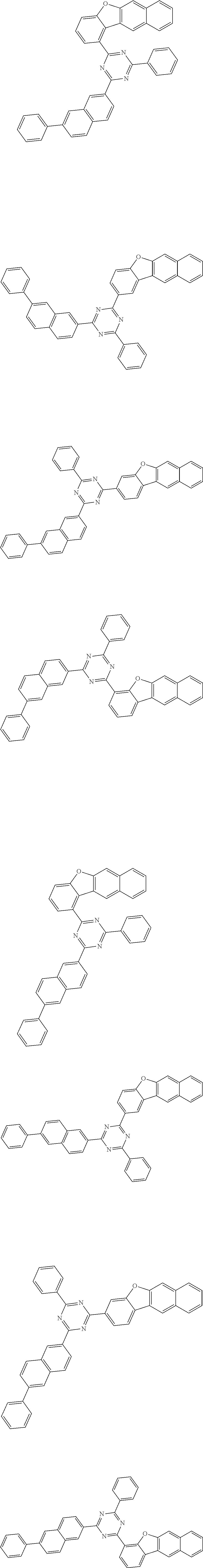

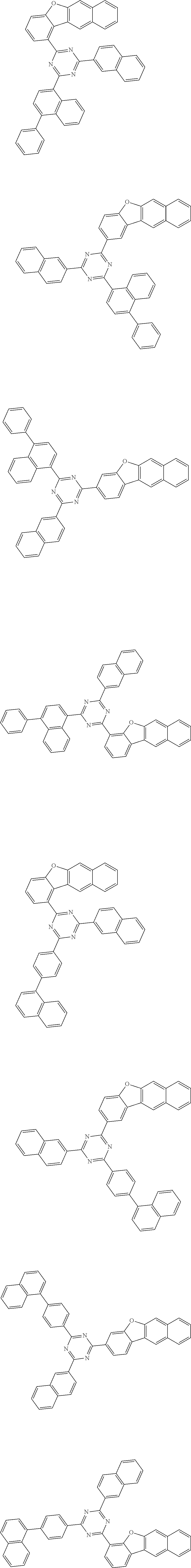

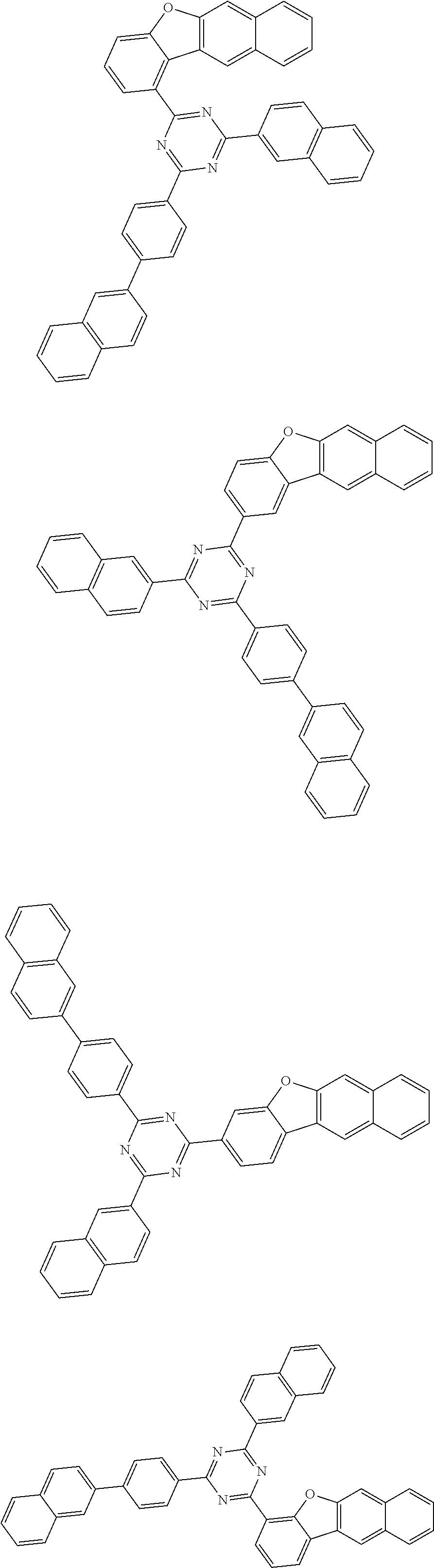 [SMILES: c1ccc2cc(-c3ccc(-c4nc(-c5ccc6ccccc6c5)nc(-c5cccc6oc7cc8ccccc8cc7c56)n4)cc3)ccc2c1 ; c1ccc2cc(-c3ccc(-c4nc(-c5ccc6ccccc6c5)nc(-c5ccc6oc7cc8ccccc8cc7c6c5)n4)cc3)ccc2c1 ; c1ccc2cc(-c3ccc(-c4nc(-c5ccc6ccccc6c5)nc(-c5ccc6c(c5)oc5cc7ccccc7cc56)n4)cc3)ccc2c1 ; c1ccc2cc(-c3ccc(-c4nc(-c5ccc6ccccc6c5)nc(-c5cccc6c5oc5cc7ccccc7cc56)n4)cc3)ccc2c1]

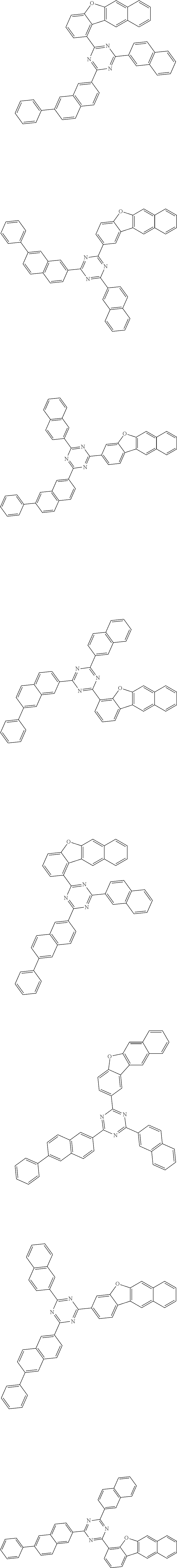

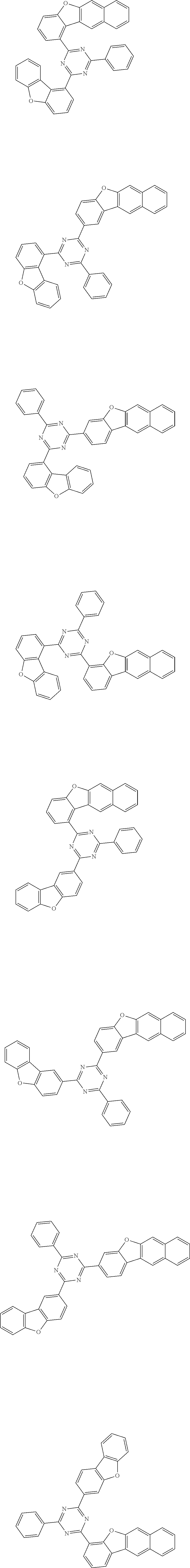

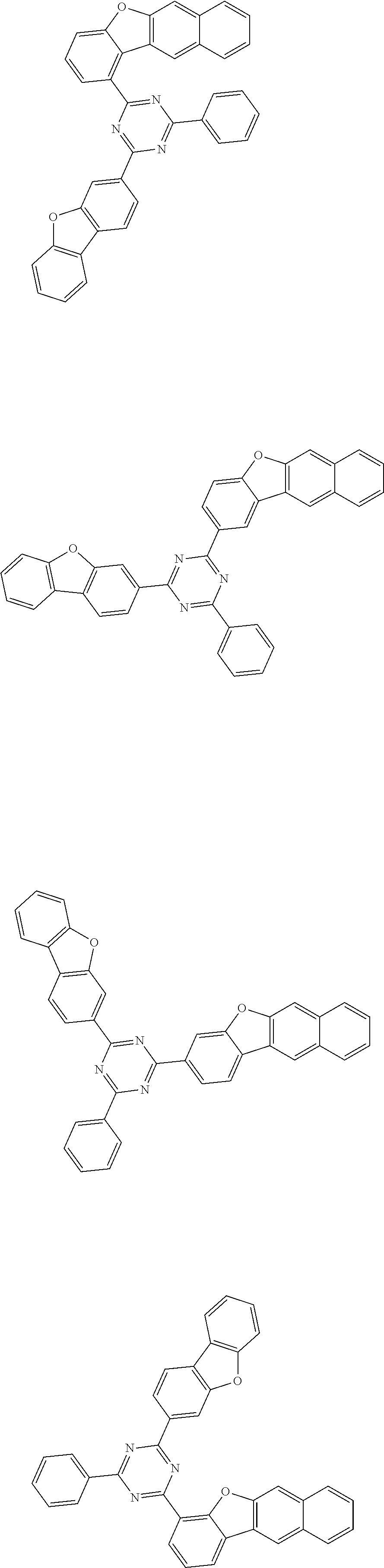 [SMILES: c1ccc(-c2nc(-c3ccc4c(c3)oc3ccccc34)nc(-c3cccc4oc5cc6ccccc6cc5c34)n2)cc1 ; c1ccc(-c2nc(-c3ccc4c(c3)oc3ccccc34)nc(-c3ccc4oc5cc6ccccc6cc5c4c3)n2)cc1 ; c1ccc(-c2nc(-c3ccc4c(c3)oc3ccccc34)nc(-c3ccc4c(c3)oc3cc5ccccc5cc34)n2)cc1 ; c1ccc(-c2nc(-c3ccc4c(c3)oc3ccccc34)nc(-c3cccc4c3oc3cc5ccccc5cc34)n2)cc1]

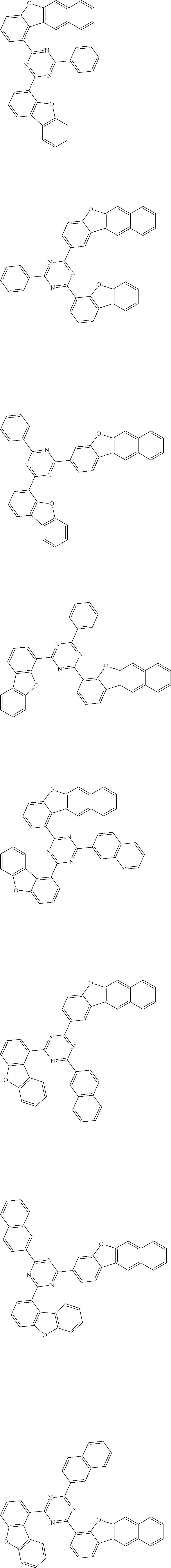

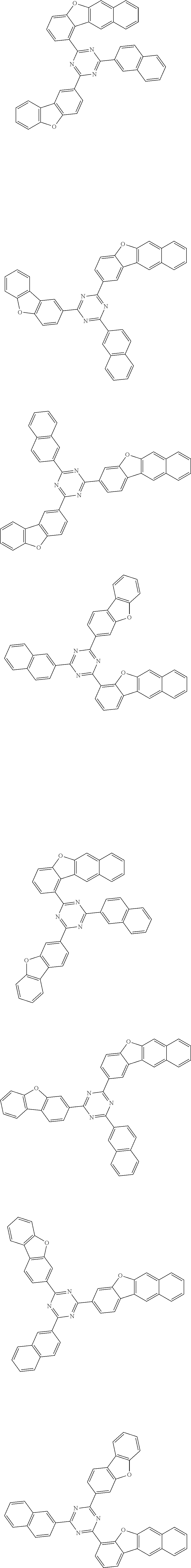

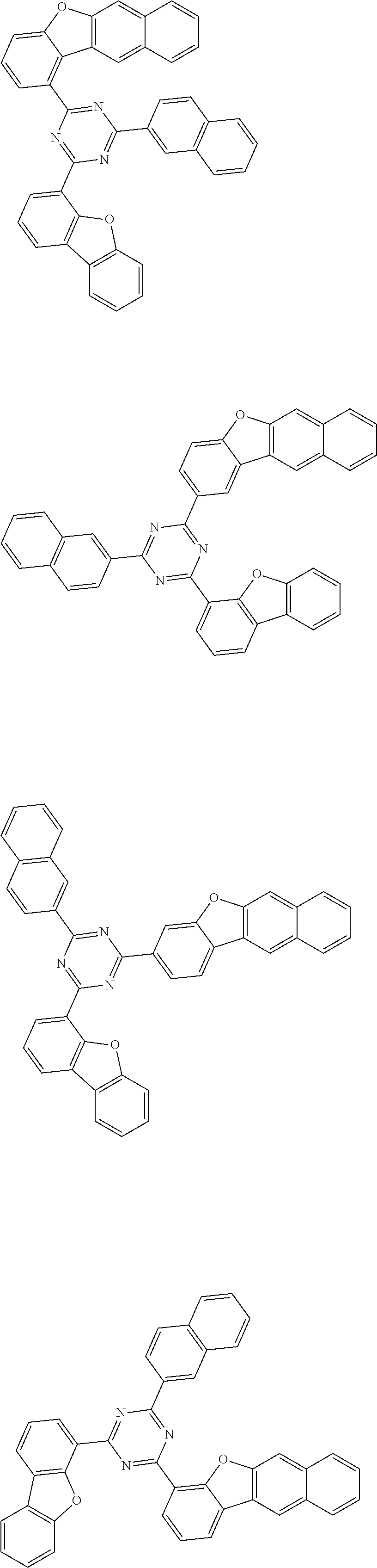 [SMILES: c1ccc2cc(-c3nc(-c4cccc5c4oc4ccccc45)nc(-c4cccc5oc6cc7ccccc7cc6c45)n3)ccc2c1 ; c1ccc2cc(-c3nc(-c4ccc5oc6cc7ccccc7cc6c5c4)nc(-c4cccc5c4oc4ccccc45)n3)ccc2c1 ; c1ccc2cc(-c3nc(-c4ccc5c(c4)oc4cc6ccccc6cc45)nc(-c4cccc5c4oc4ccccc45)n3)ccc2c1 ; c1ccc2cc(-c3nc(-c4cccc5c4oc4ccccc45)nc(-c4cccc5c4oc4cc6ccccc6cc45)n3)ccc2c1]

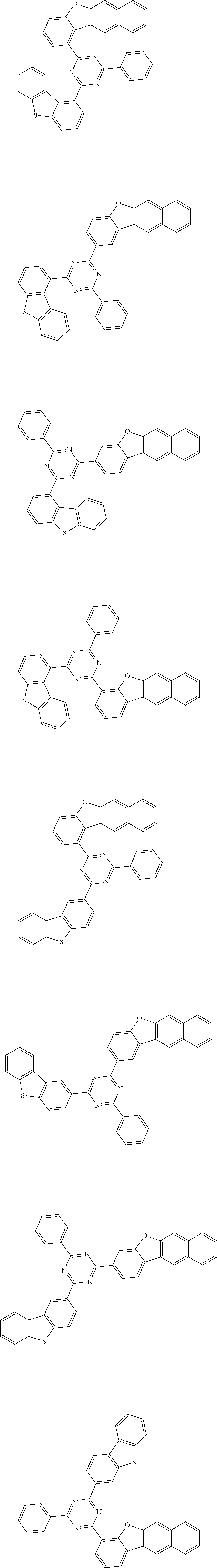

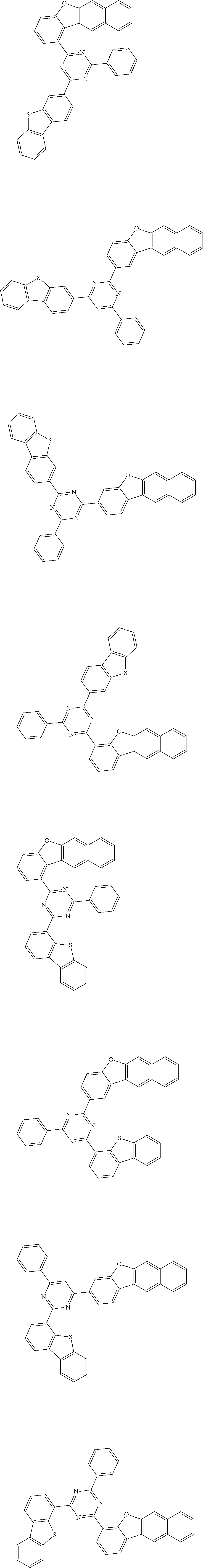

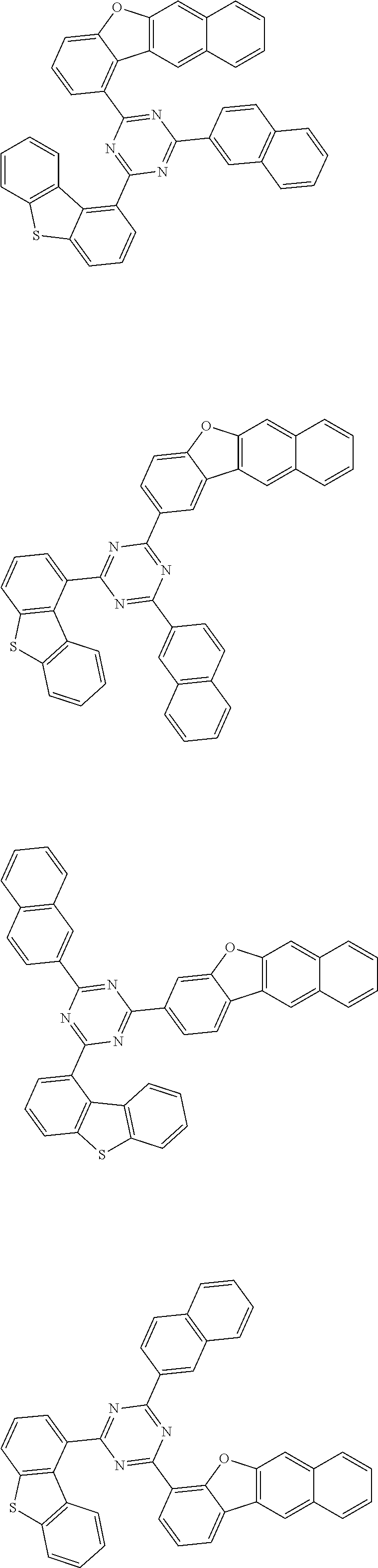 [SMILES: c1ccc2cc(-c3nc(-c4cccc5oc6cc7ccccc7cc6c45)nc(-c4cccc5sc6ccccc6c45)n3)ccc2c1 ; c1ccc2cc(-c3nc(-c4ccc5oc6cc7ccccc7cc6c5c4)nc(-c4cccc5sc6ccccc6c45)n3)ccc2c1 ; c1ccc2cc(-c3nc(-c4ccc5c(c4)oc4cc6ccccc6cc45)nc(-c4cccc5sc6ccccc6c45)n3)ccc2c1 ; c1ccc2cc(-c3nc(-c4cccc5c4oc4cc6ccccc6cc45)nc(-c4cccc5sc6ccccc6c45)n3)ccc2c1]

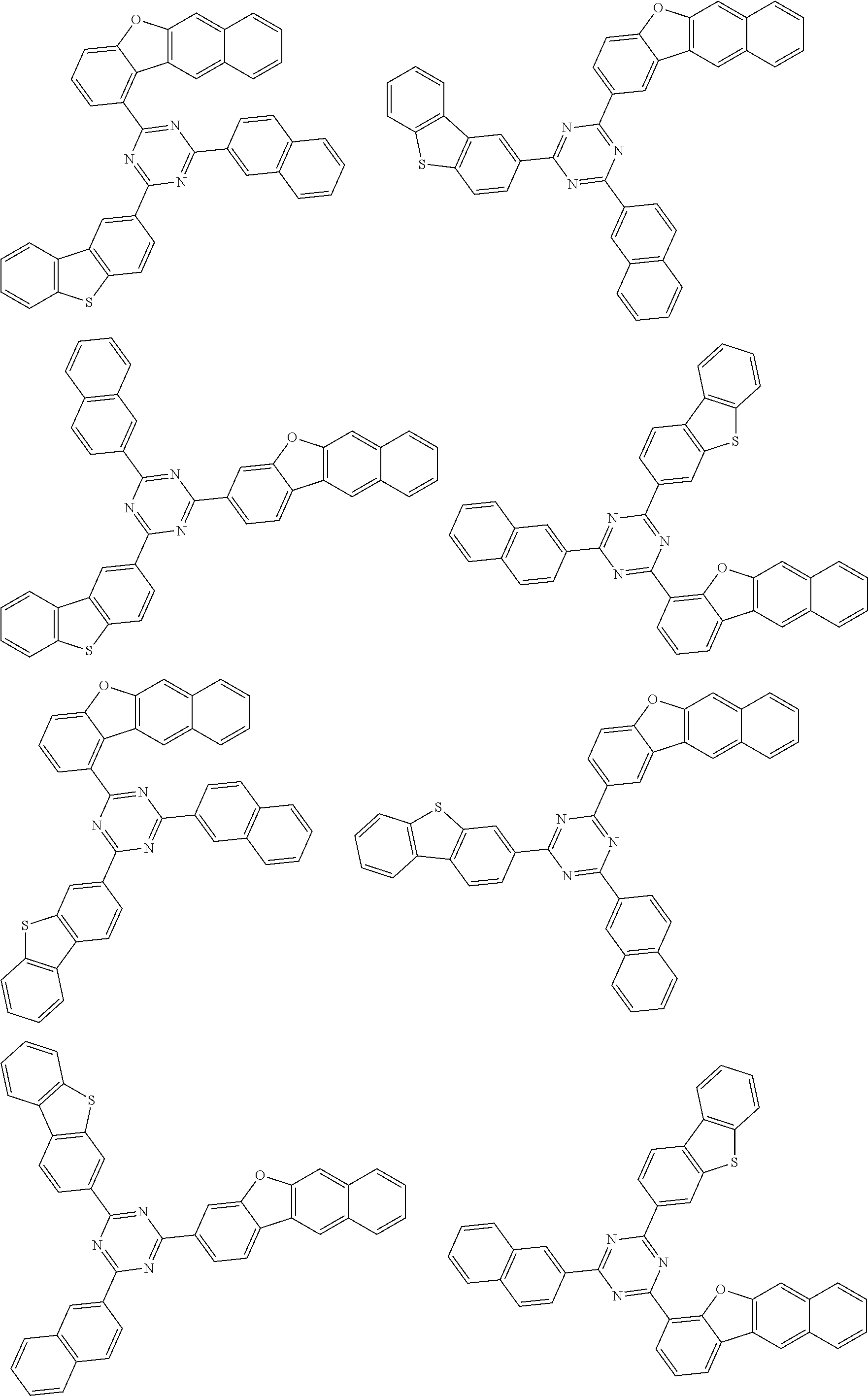

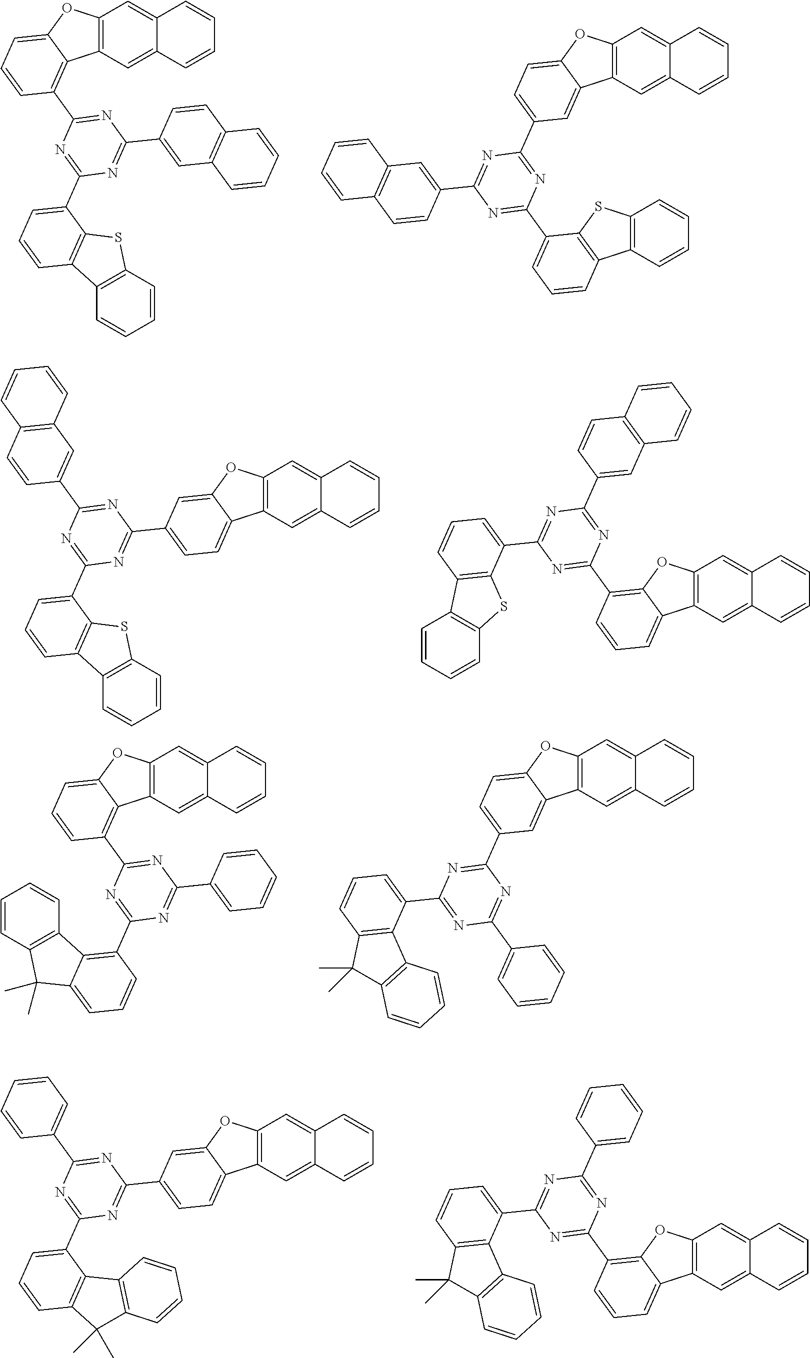

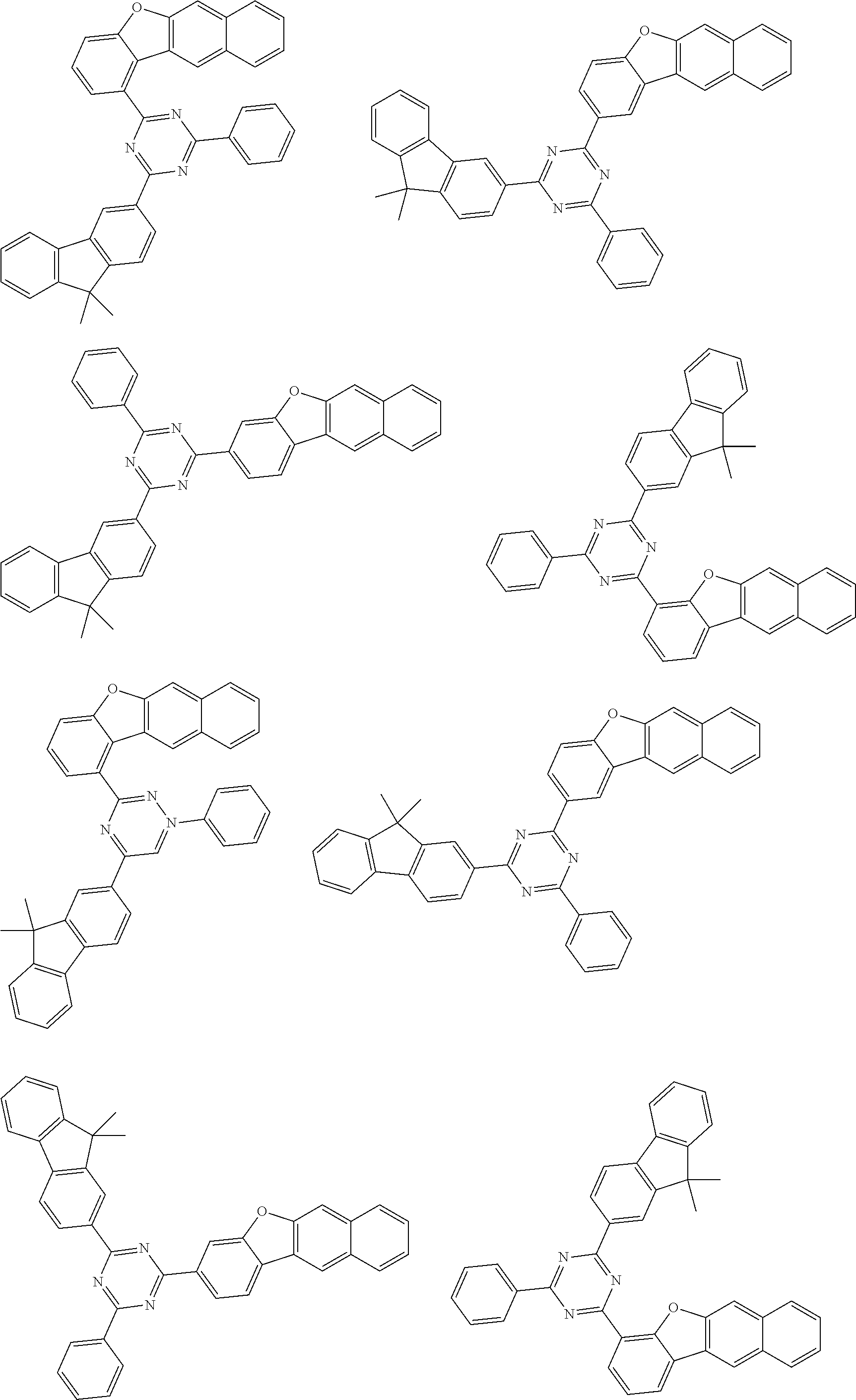

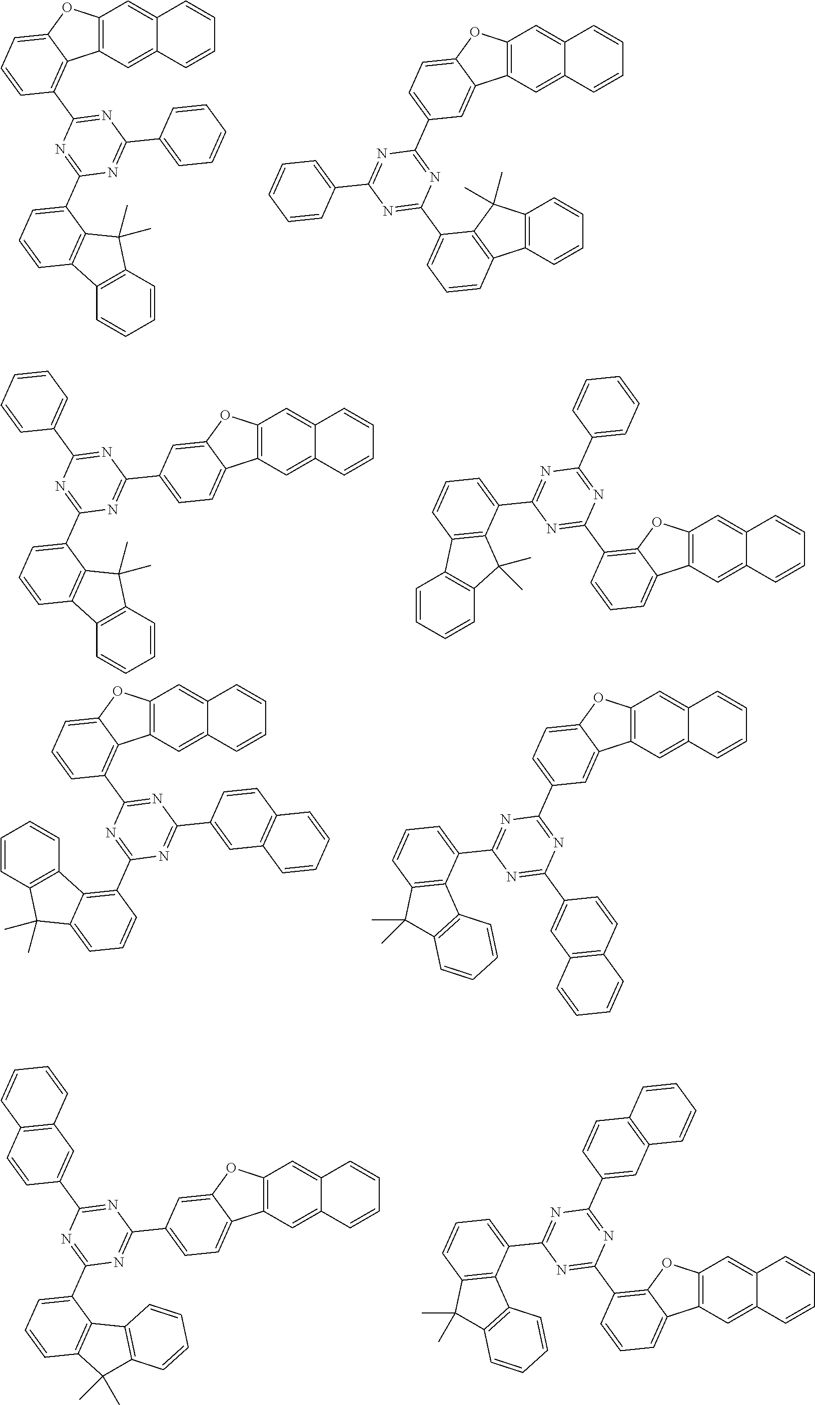

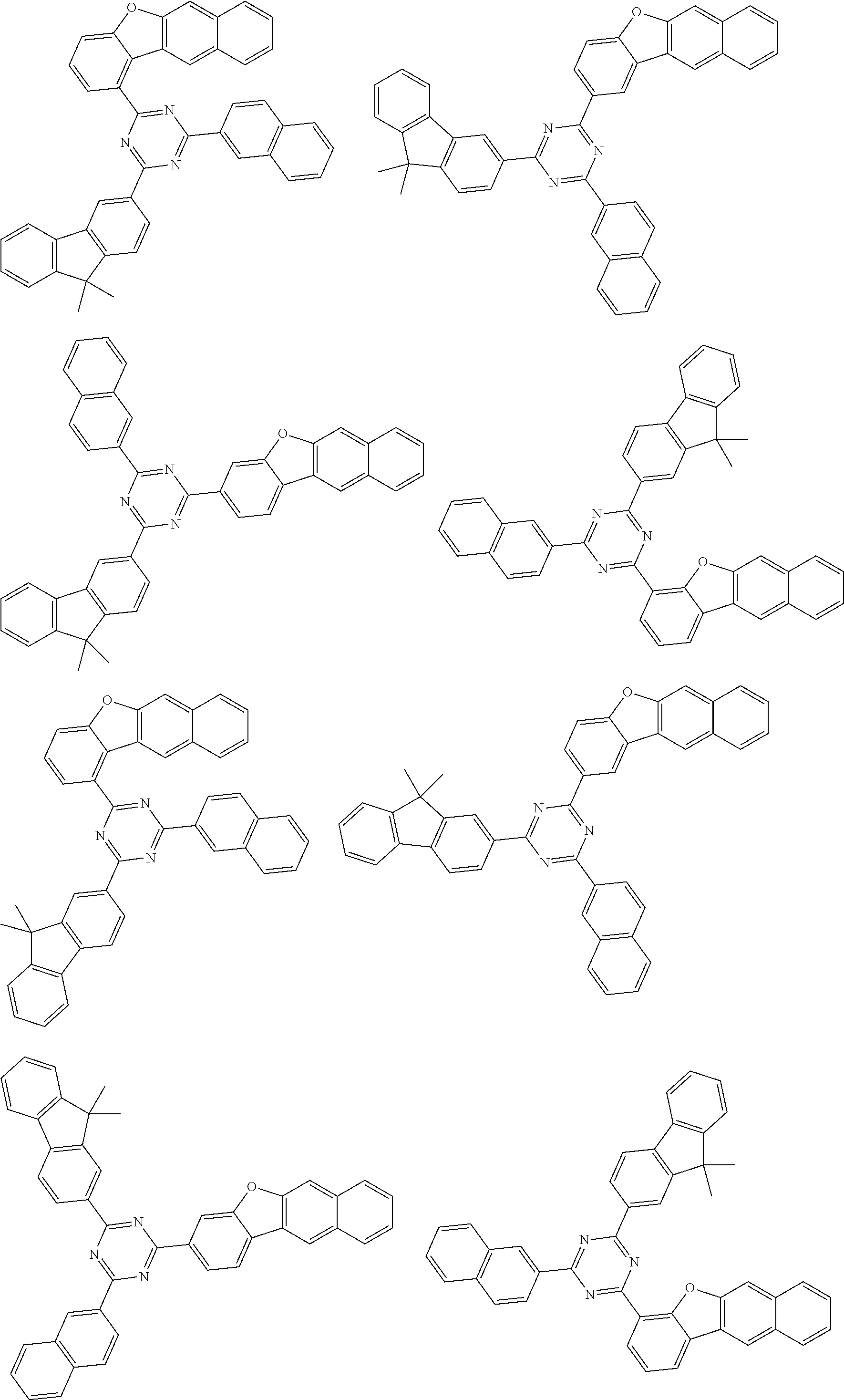

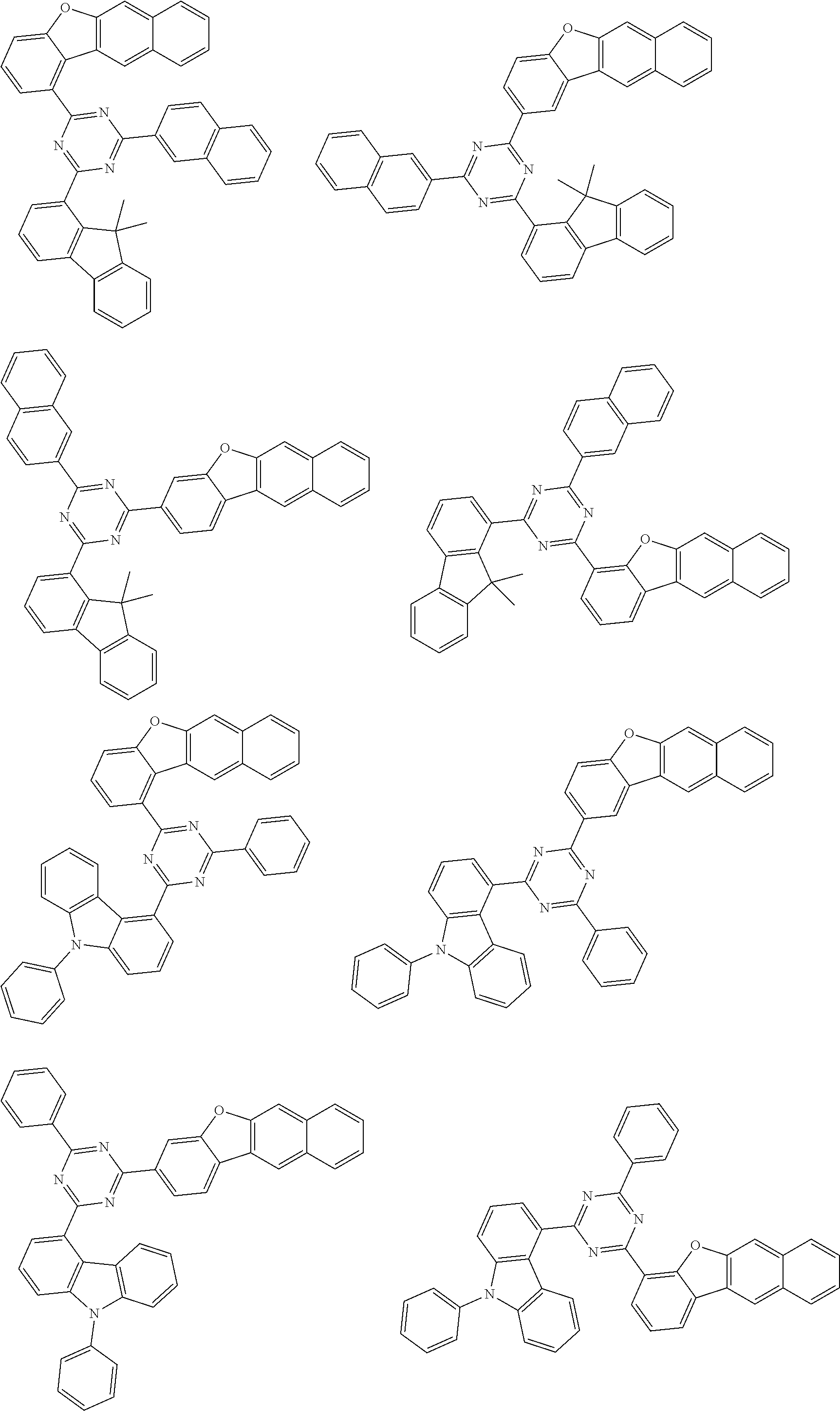

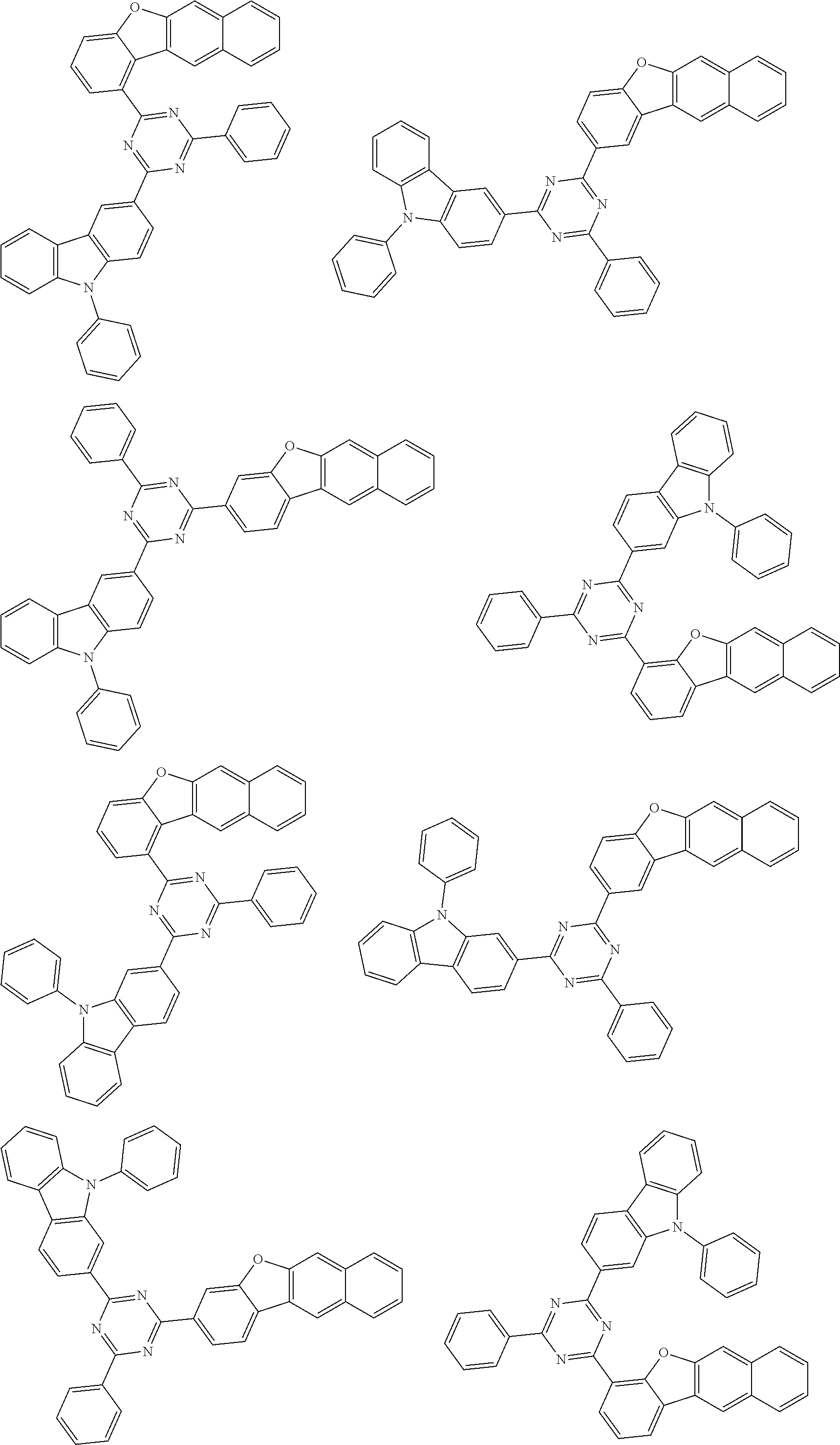

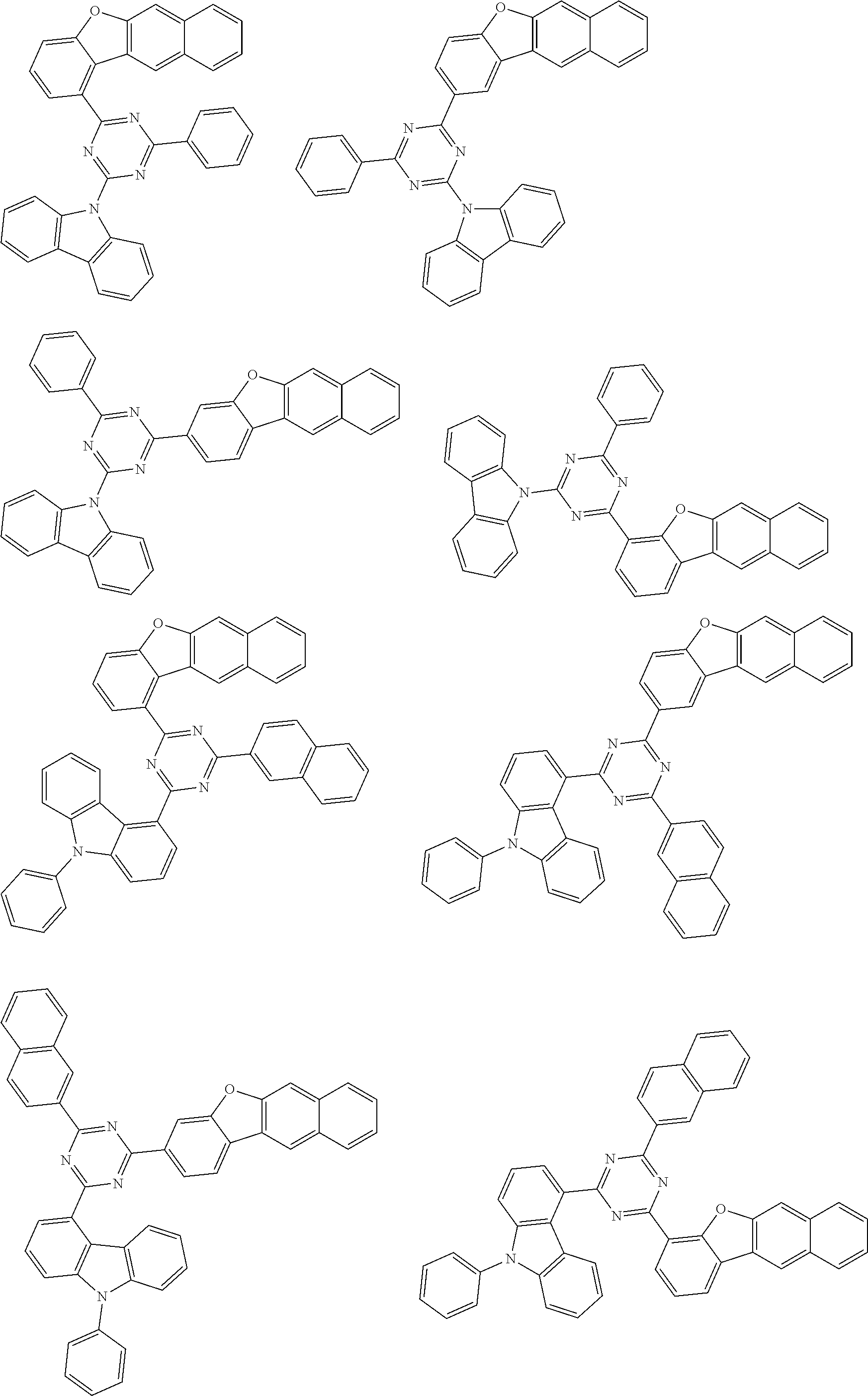

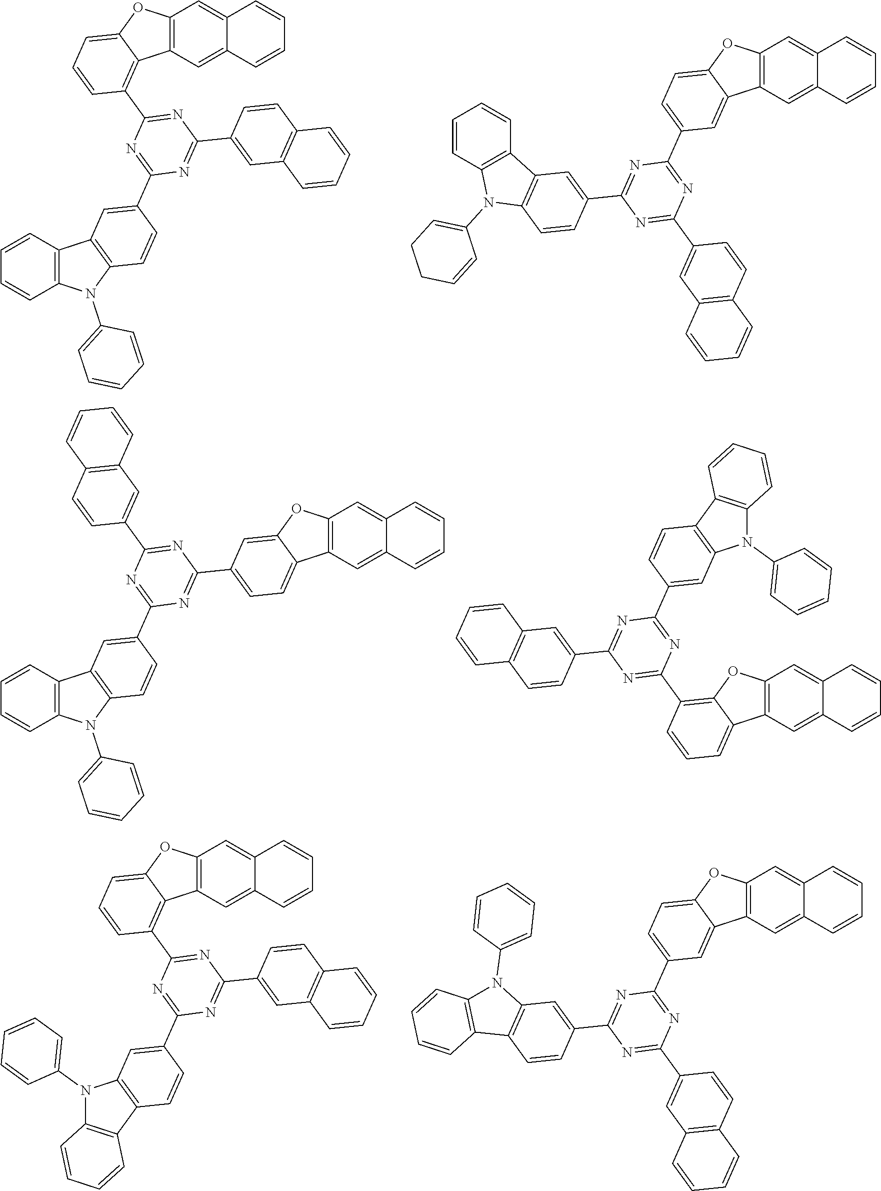

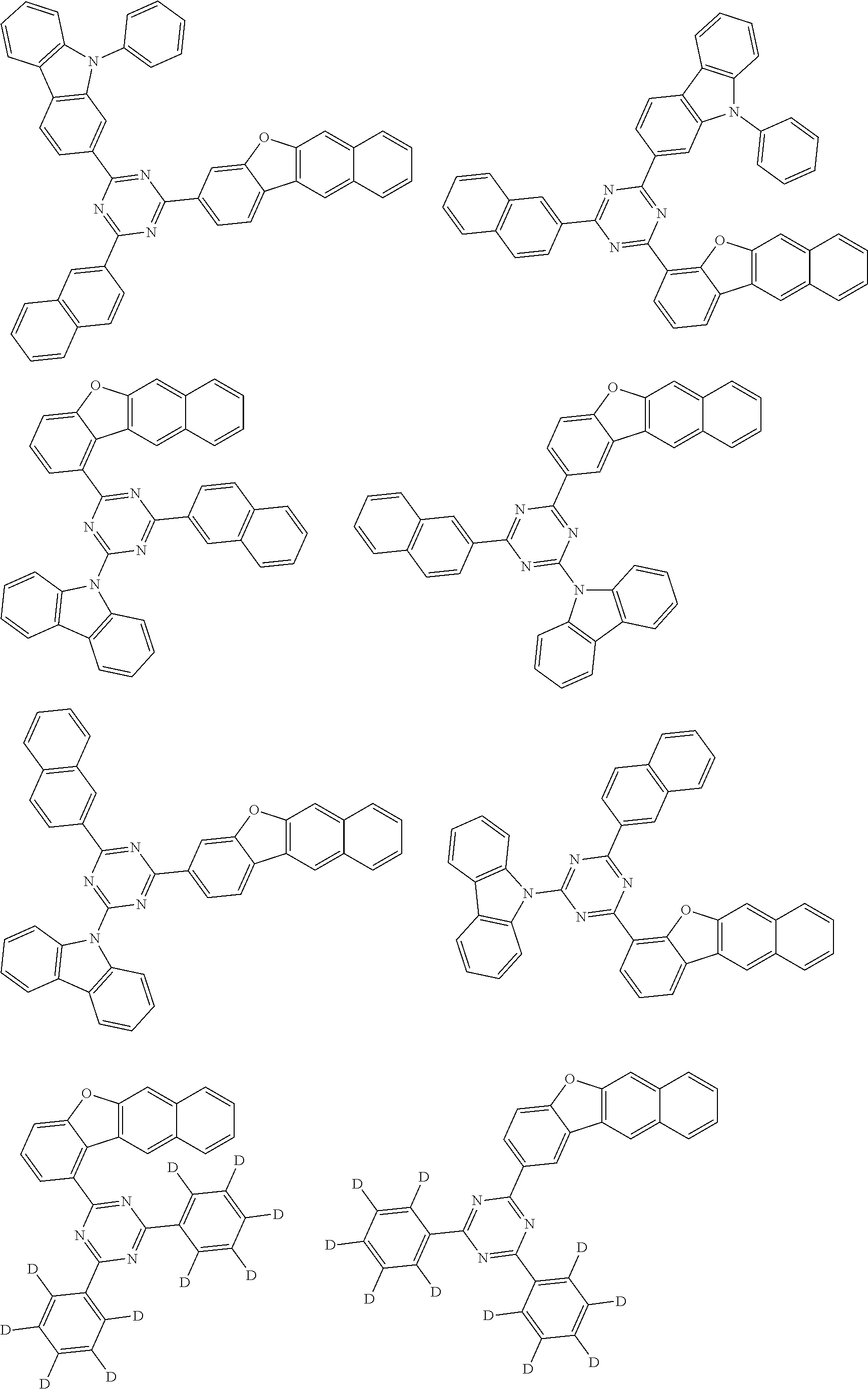

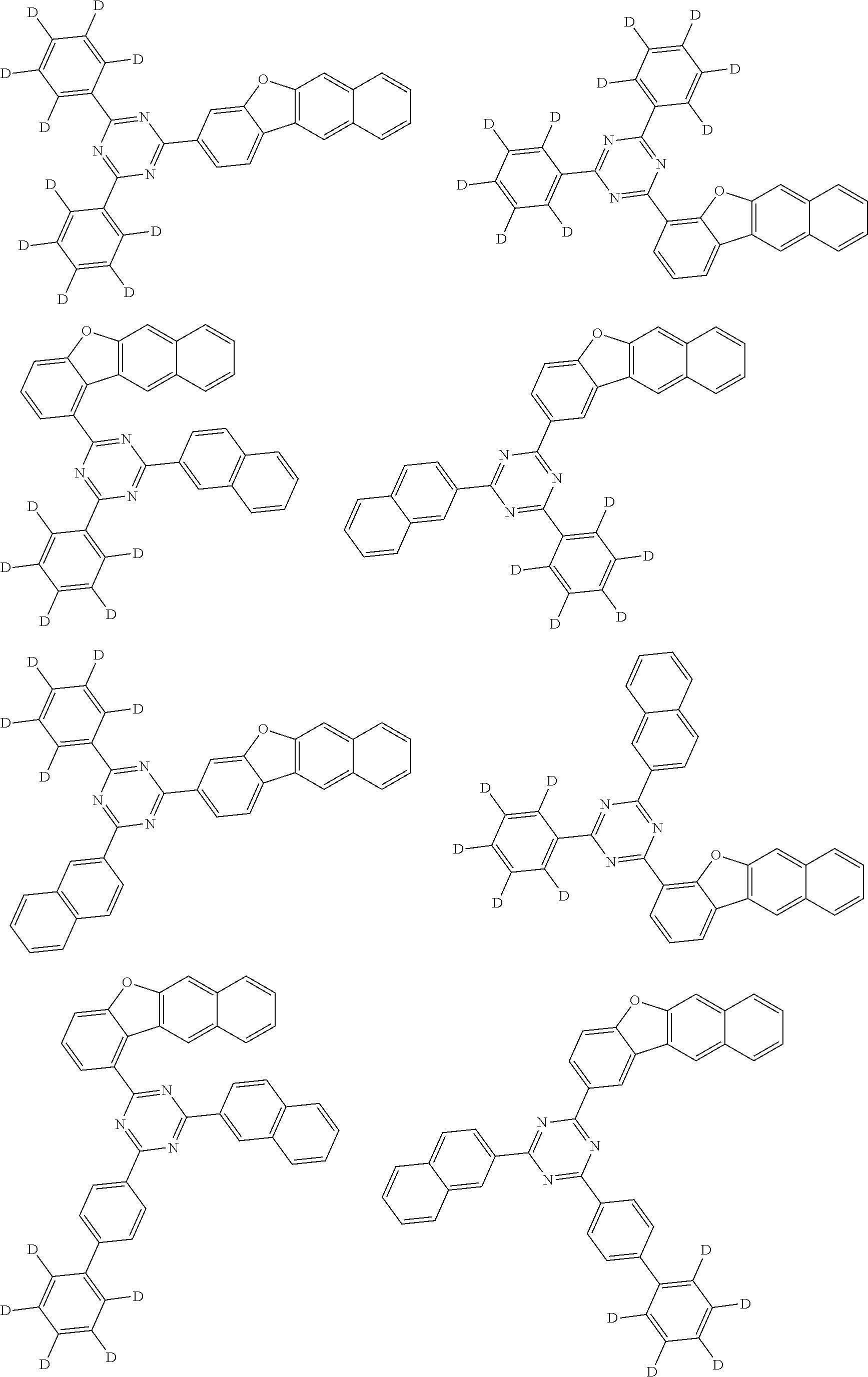

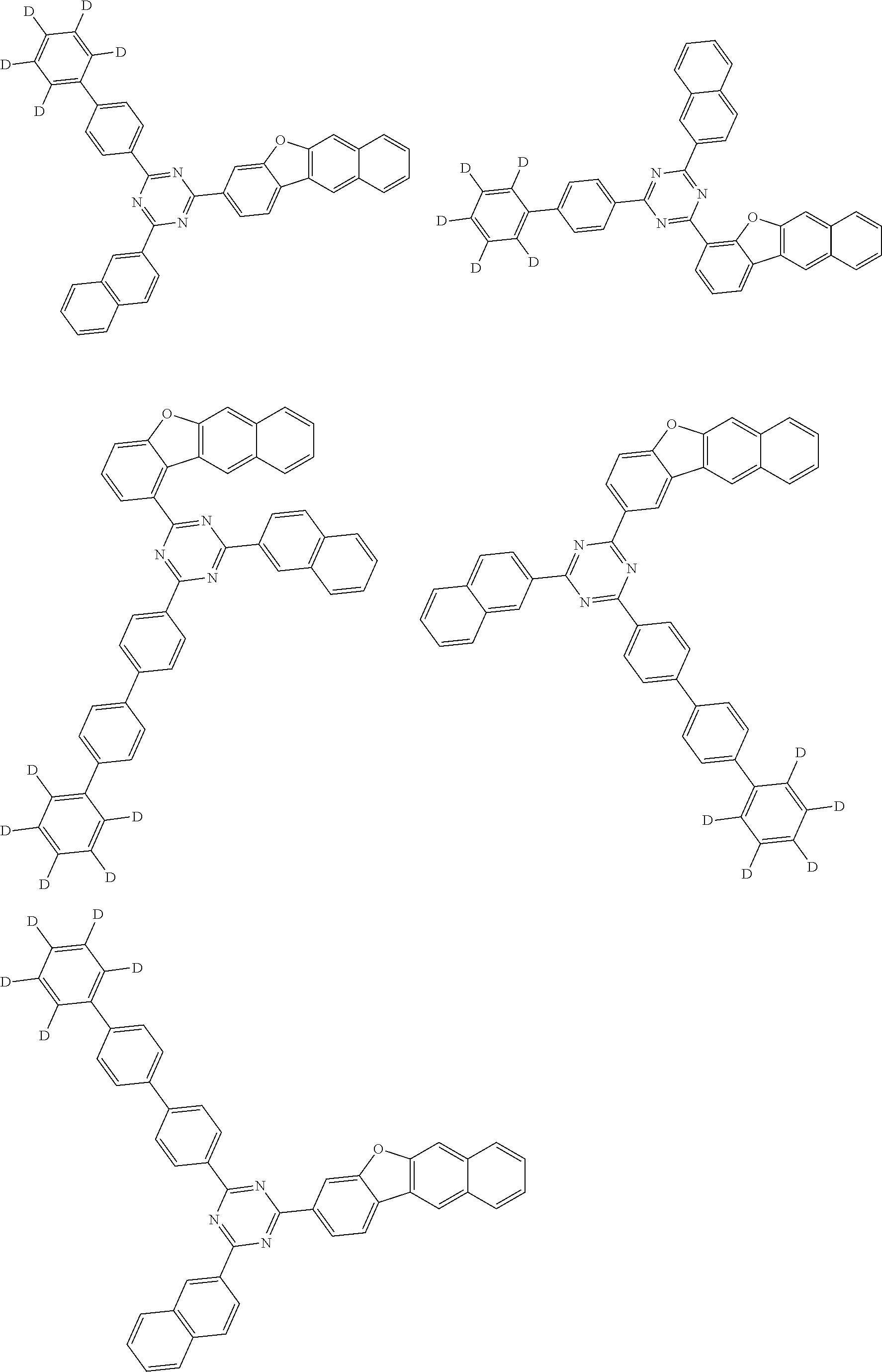

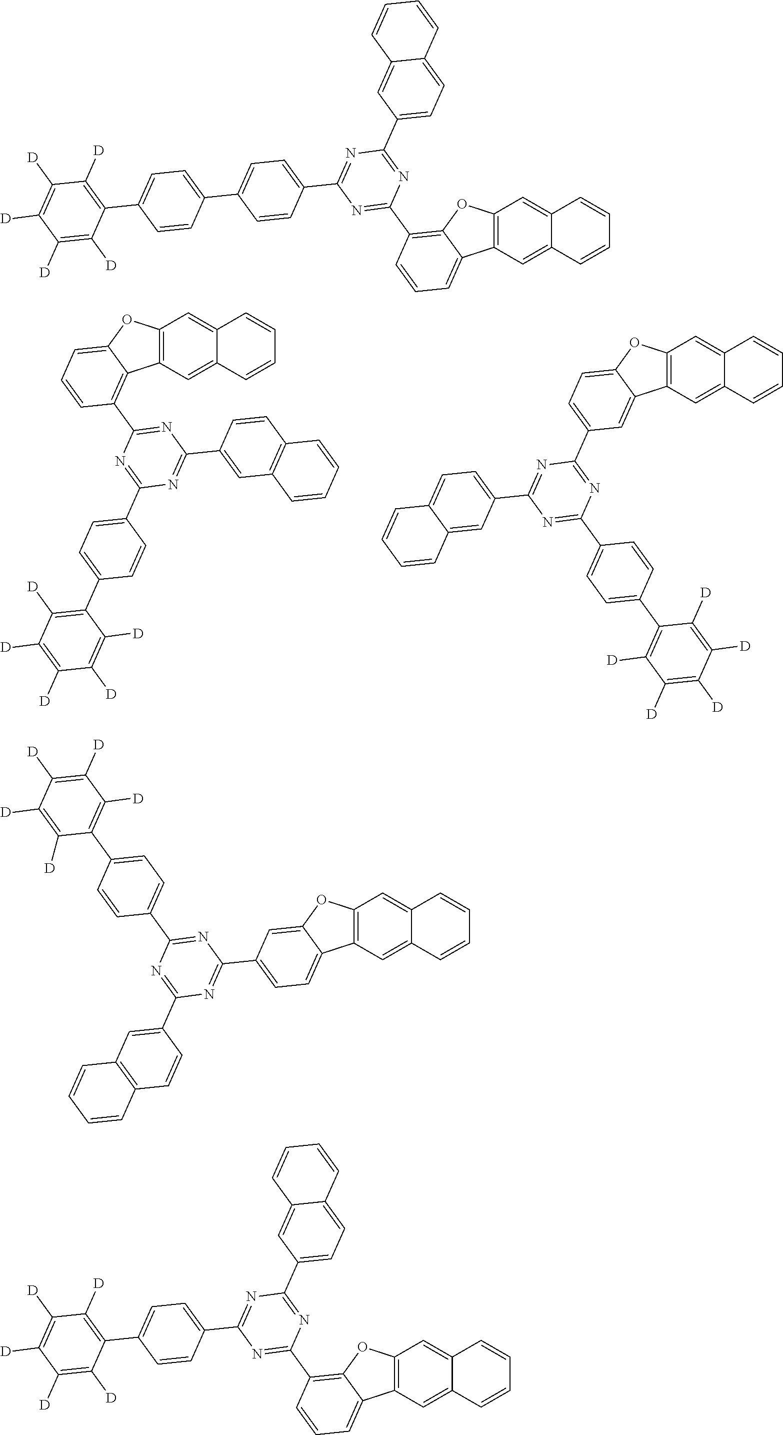

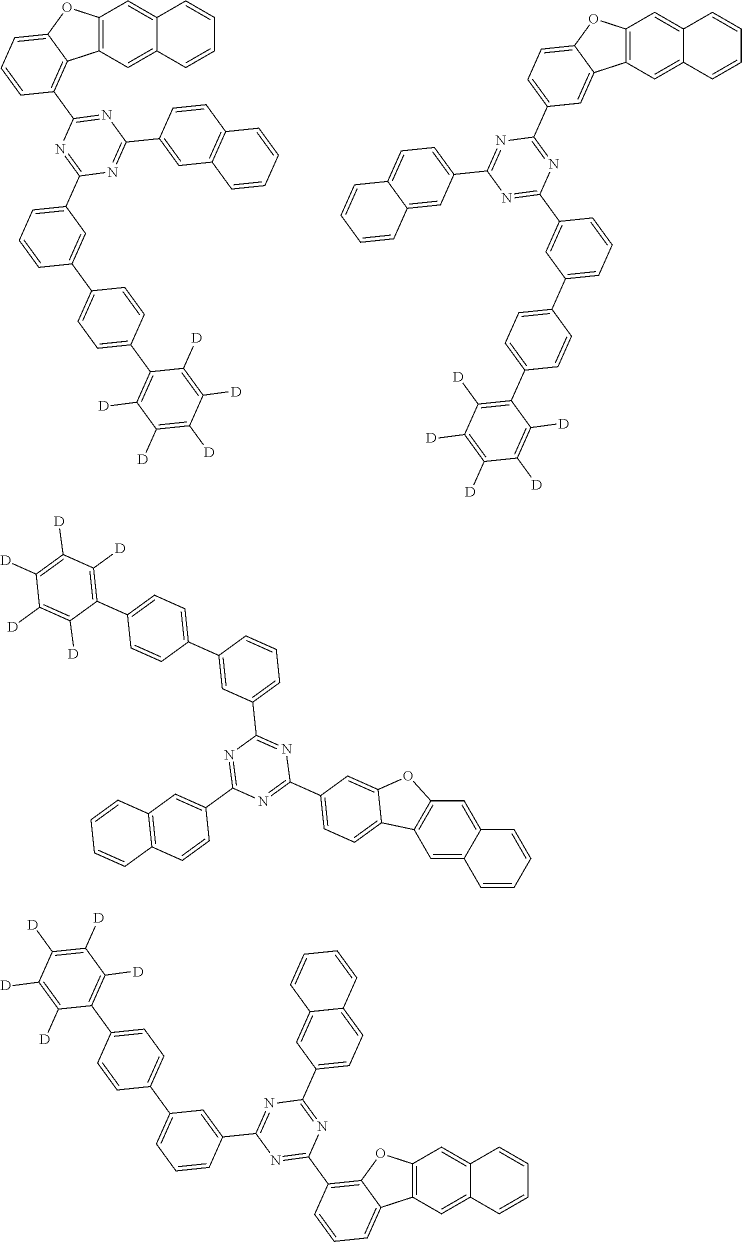 [SMILES: [2H]c1c([2H])c([2H])c(-c2ccc(-c3cccc(-c4nc(-c5ccc6ccccc6c5)nc(-c5cccc6oc7cc8ccccc8cc7c56)n4)c3)cc2)c([2H])c1[2H] ; [2H]c1c([2H])c([2H])c(-c2ccc(-c3cccc(-c4nc(-c5ccc6ccccc6c5)nc(-c5ccc6oc7cc8ccccc8cc7c6c5)n4)c3)cc2)c([2H])c1[2H] ; [2H]c1c([2H])c([2H])c(-c2ccc(-c3cccc(-c4nc(-c5ccc6ccccc6c5)nc(-c5ccc6c(c5)oc5cc7ccccc7cc56)n4)c3)cc2)c([2H])c1[2H] ; [2H]c1c([2H])c([2H])c(-c2ccc(-c3cccc(-c4nc(-c5ccc6ccccc6c5)nc(-c5cccc6c5oc5cc7ccccc7cc56)n4)c3)cc2)c([2H])c1[2H]]

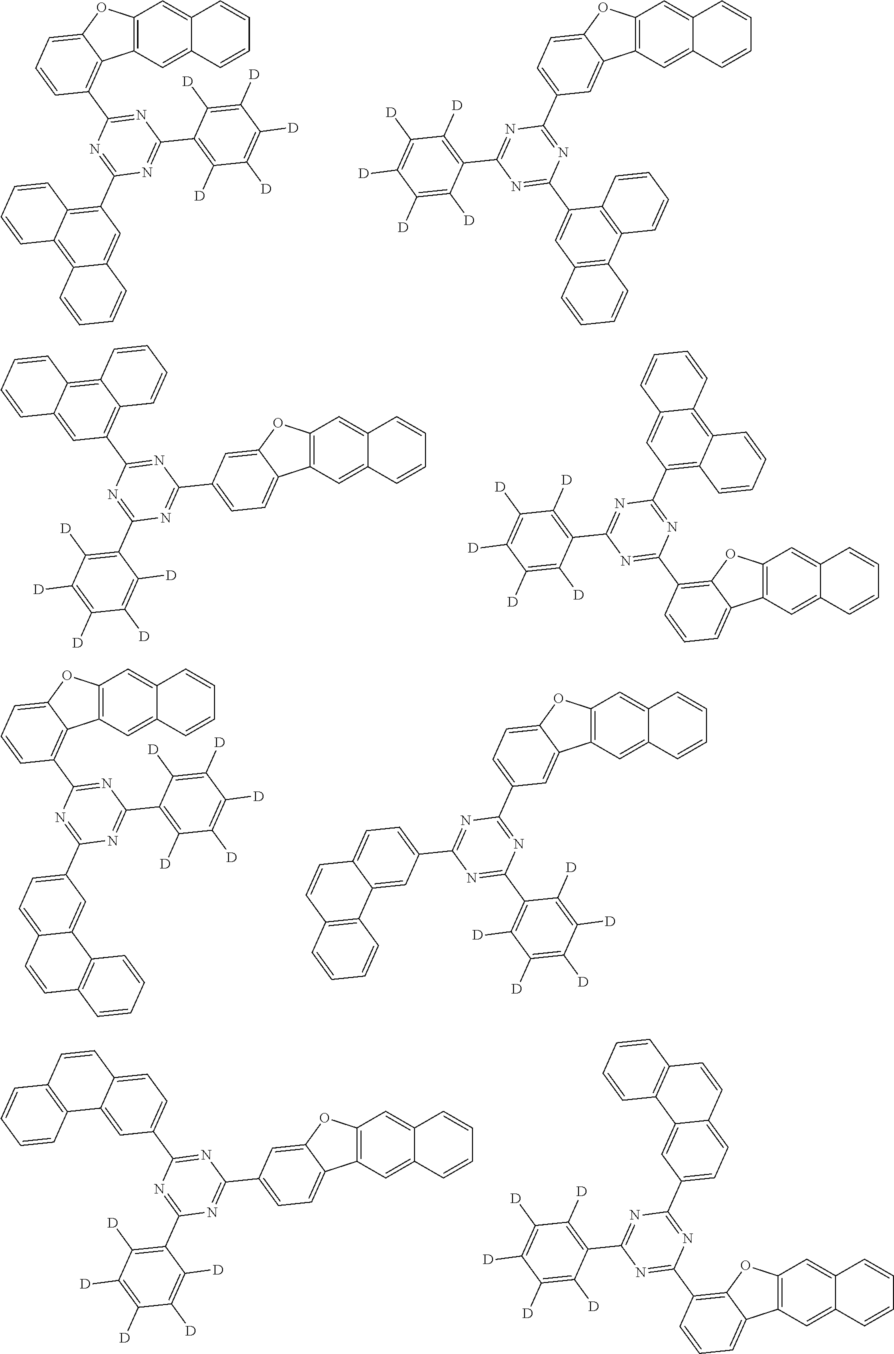

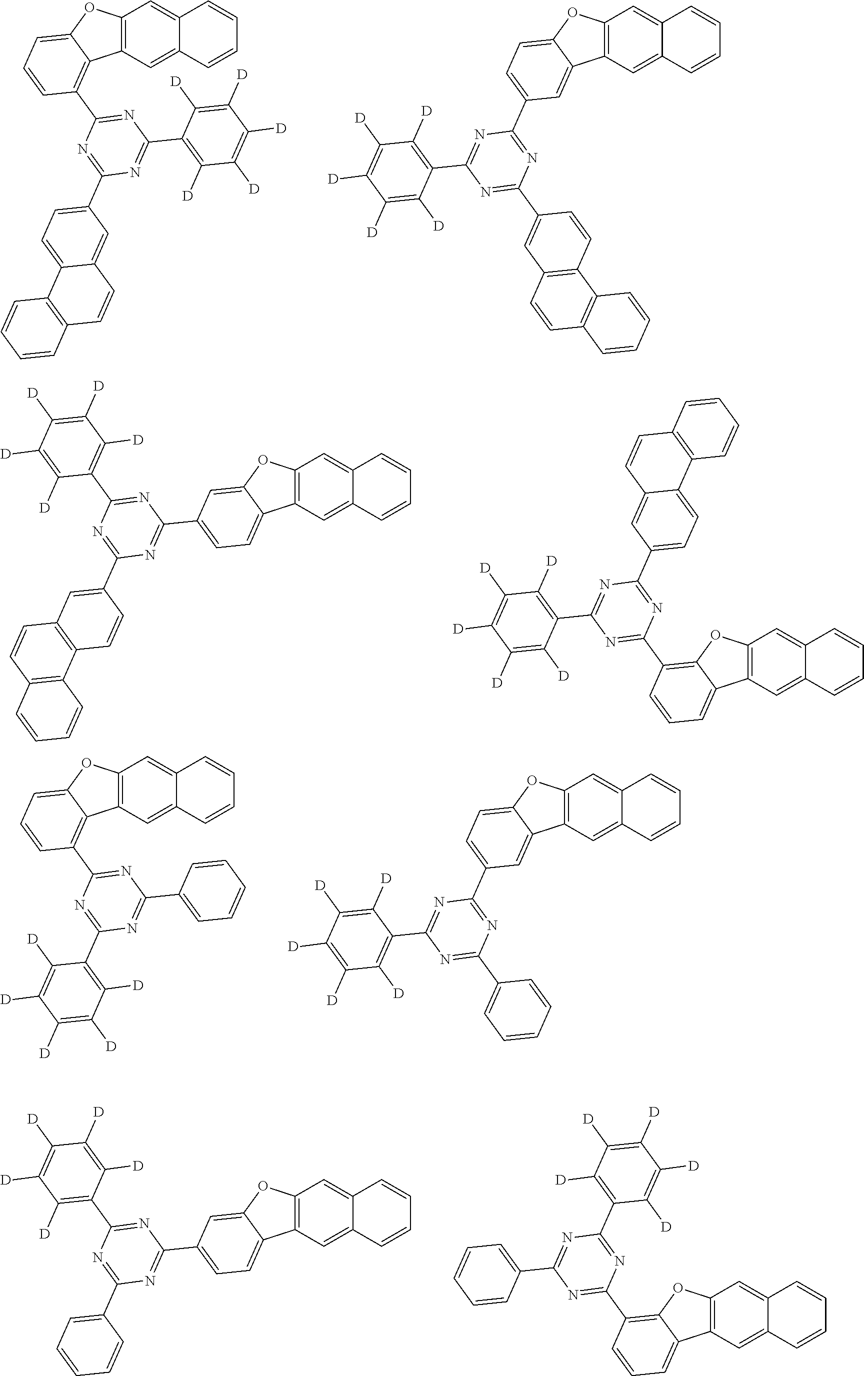

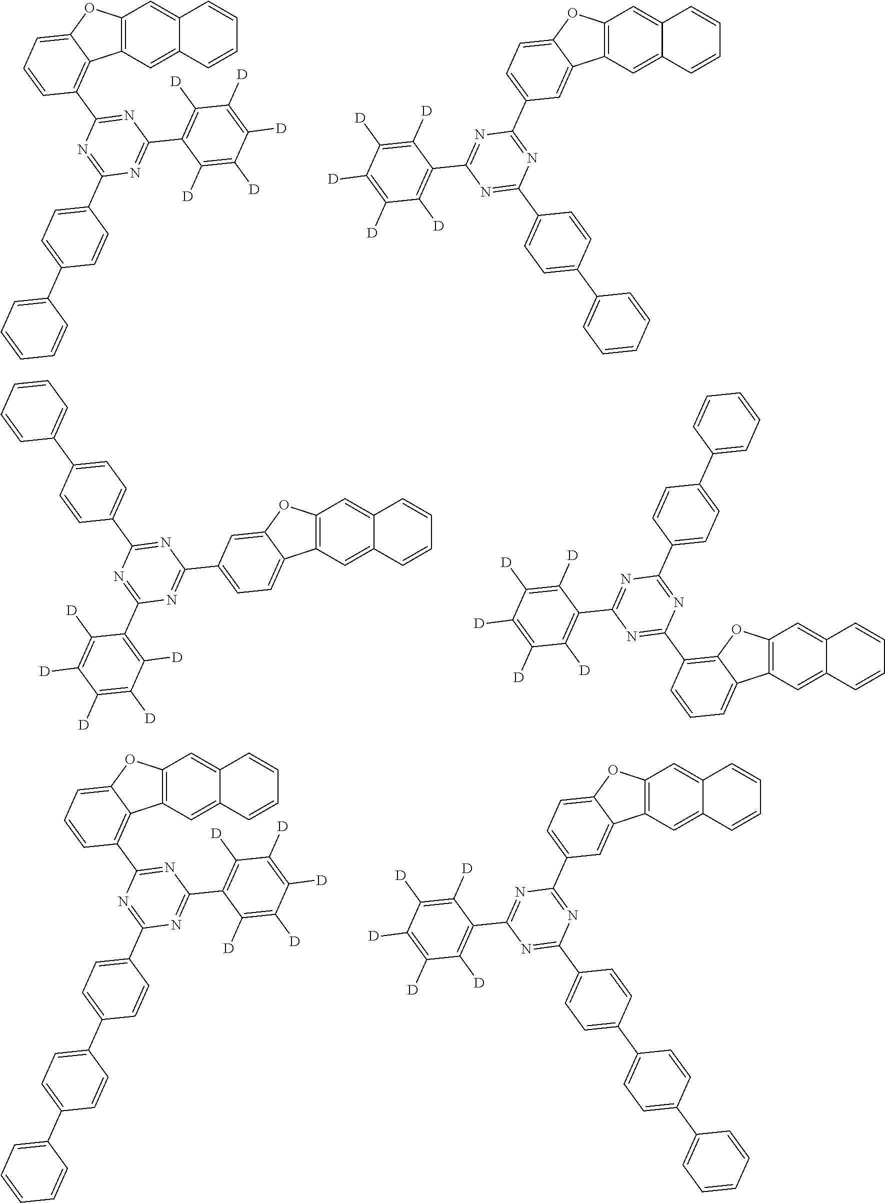

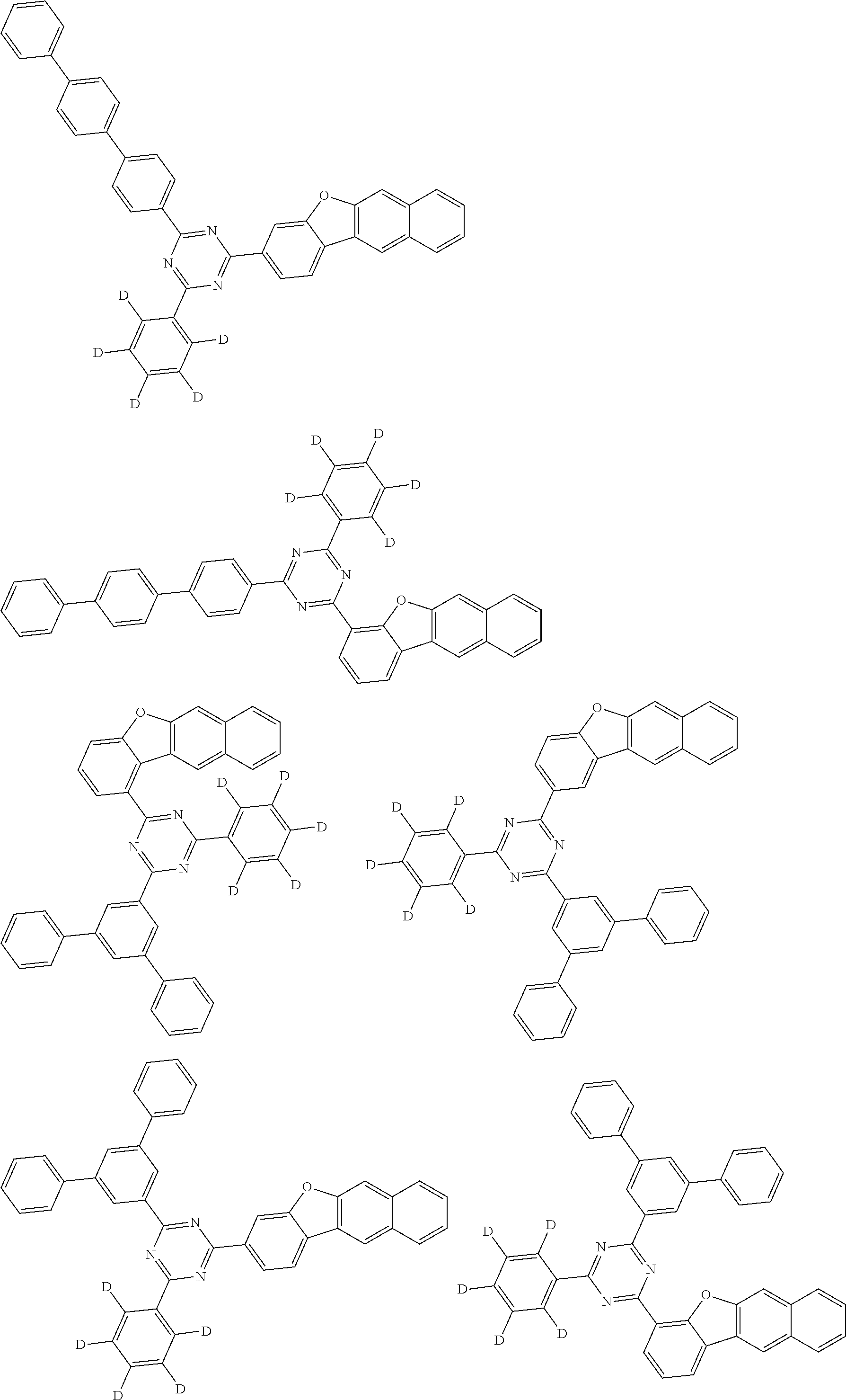

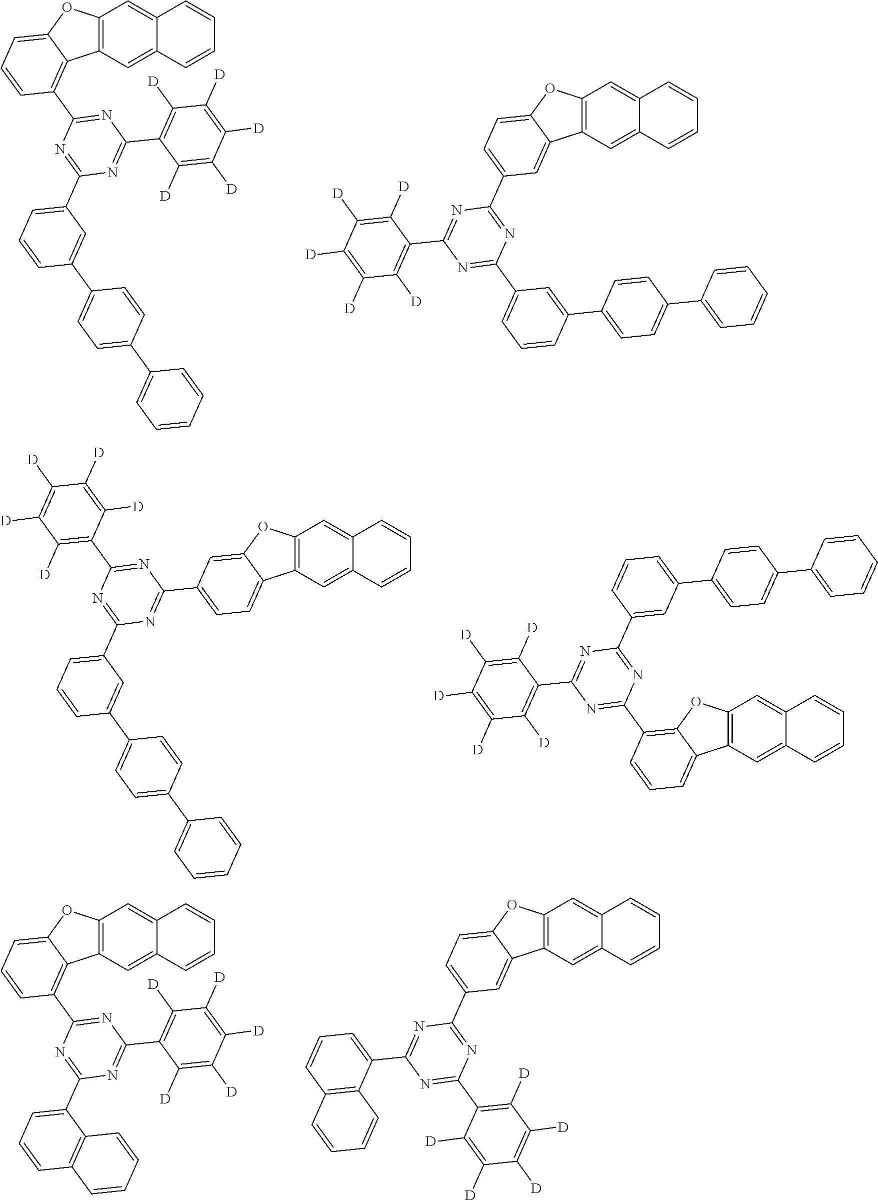

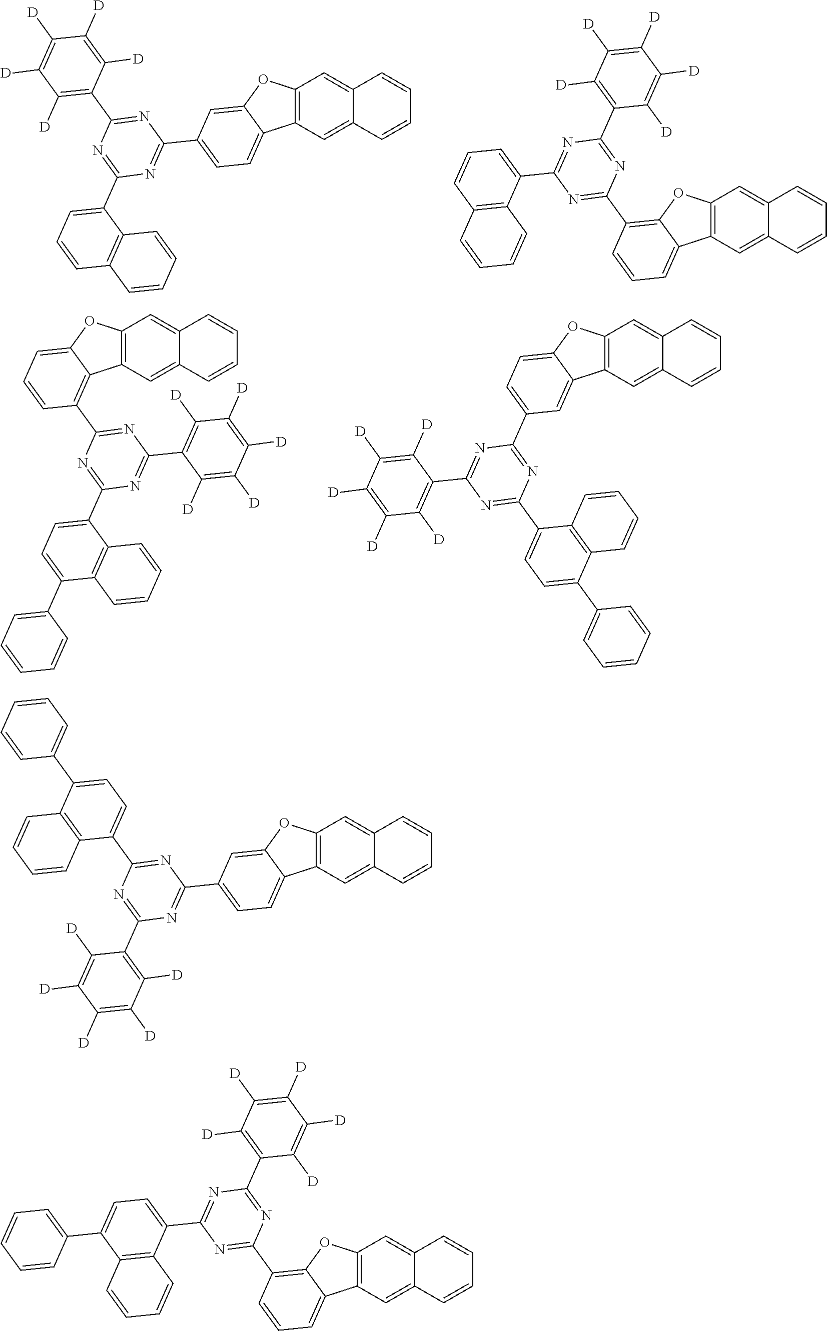

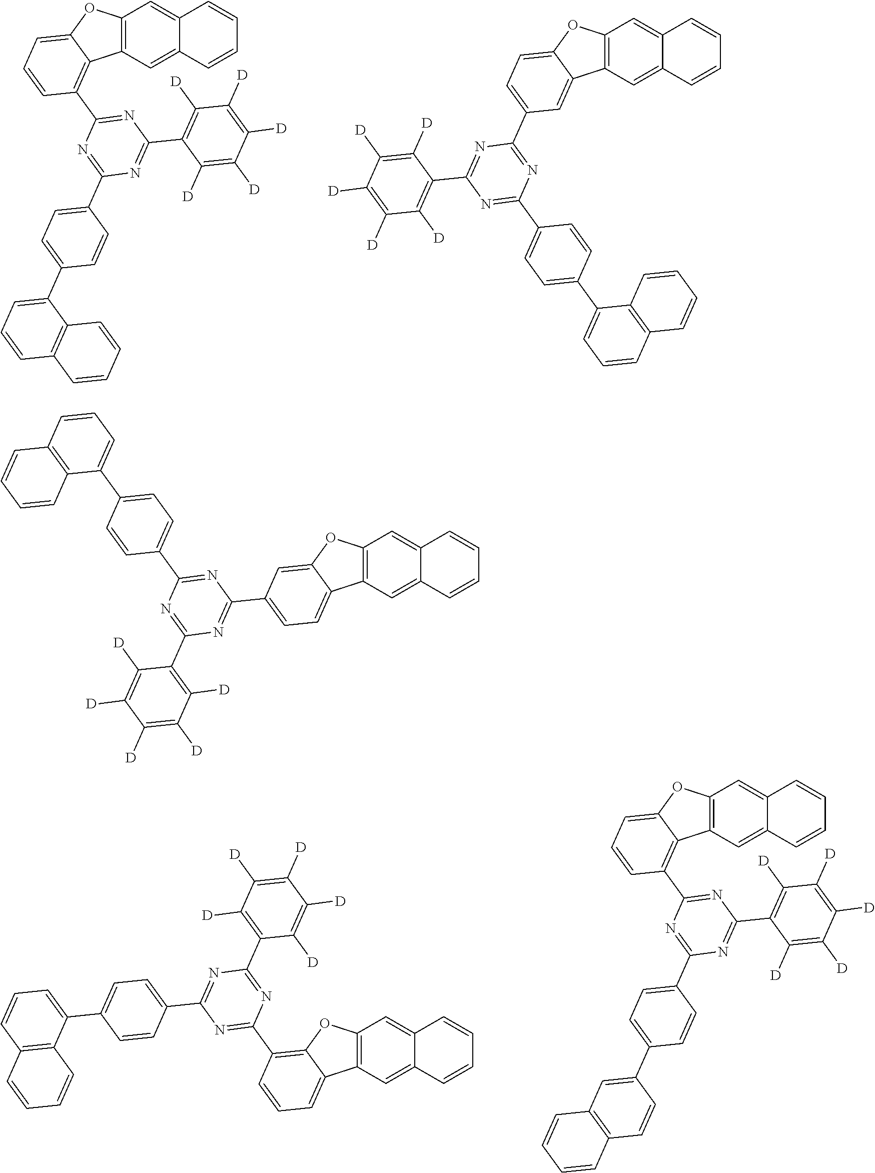

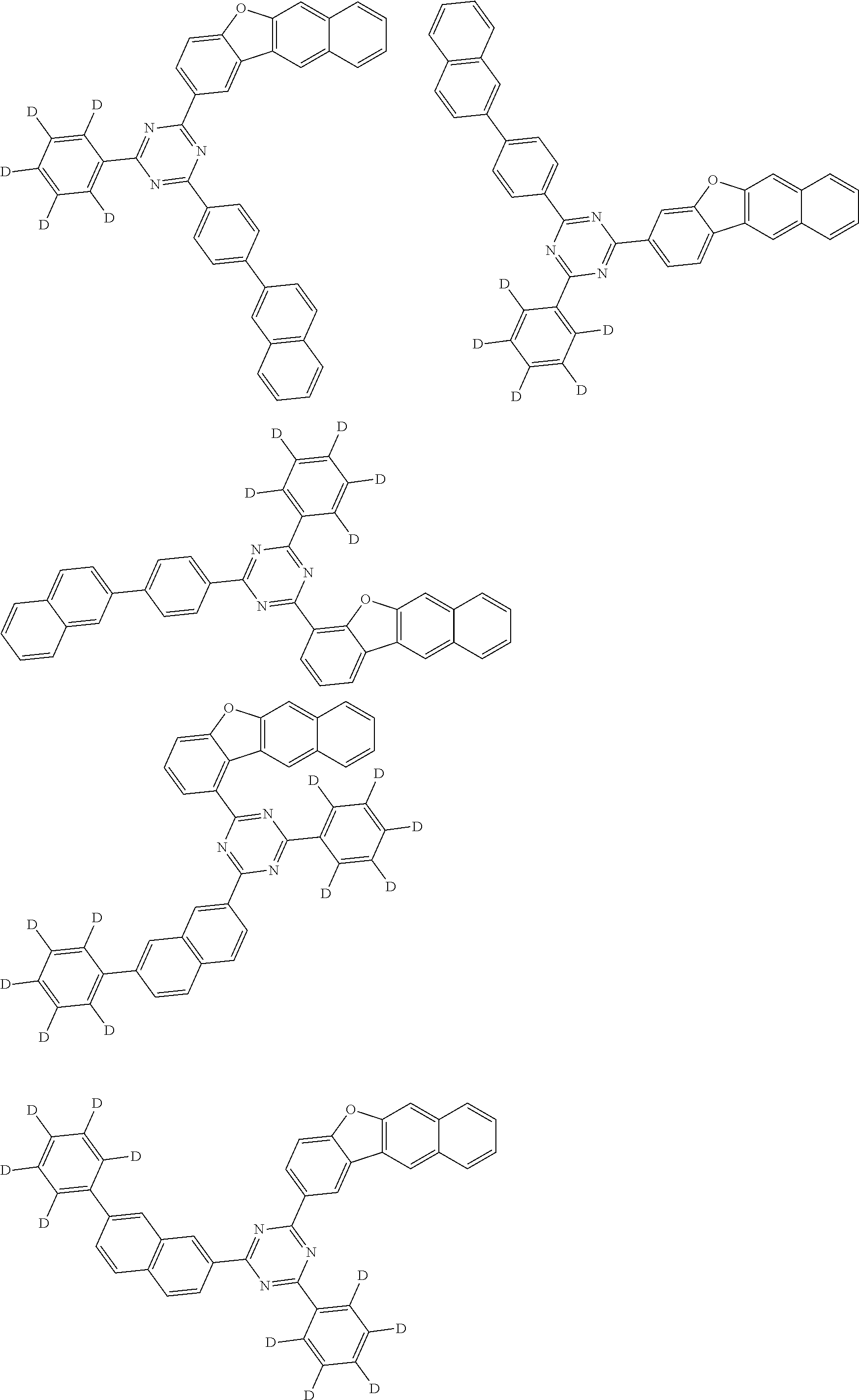

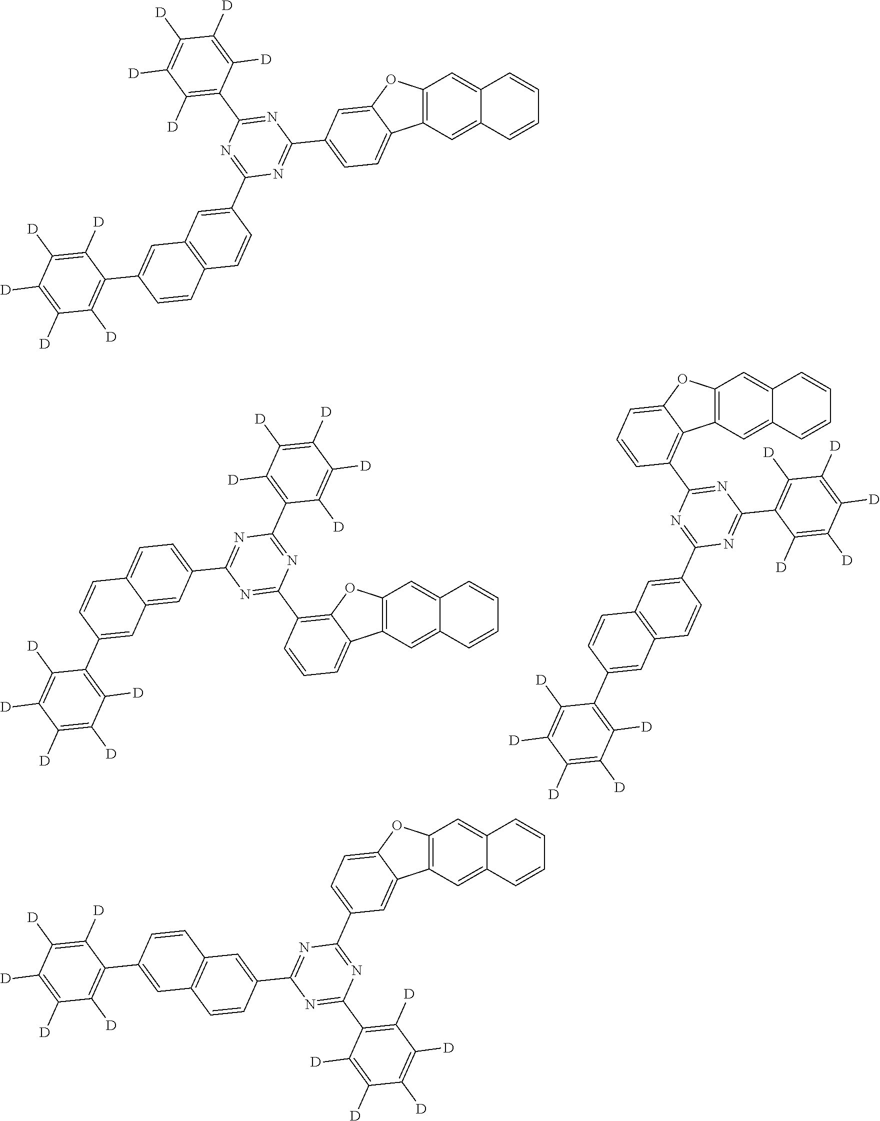

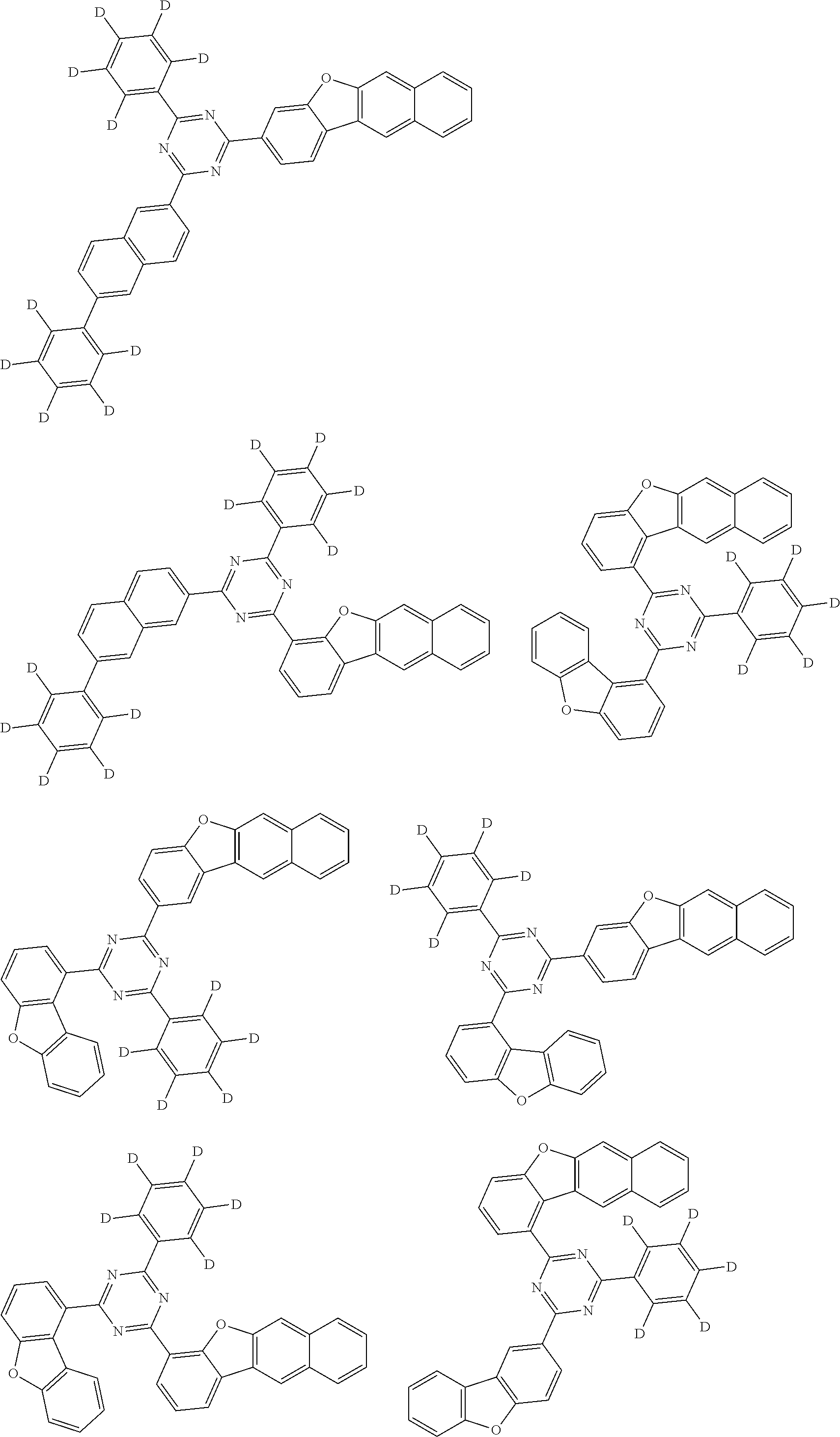

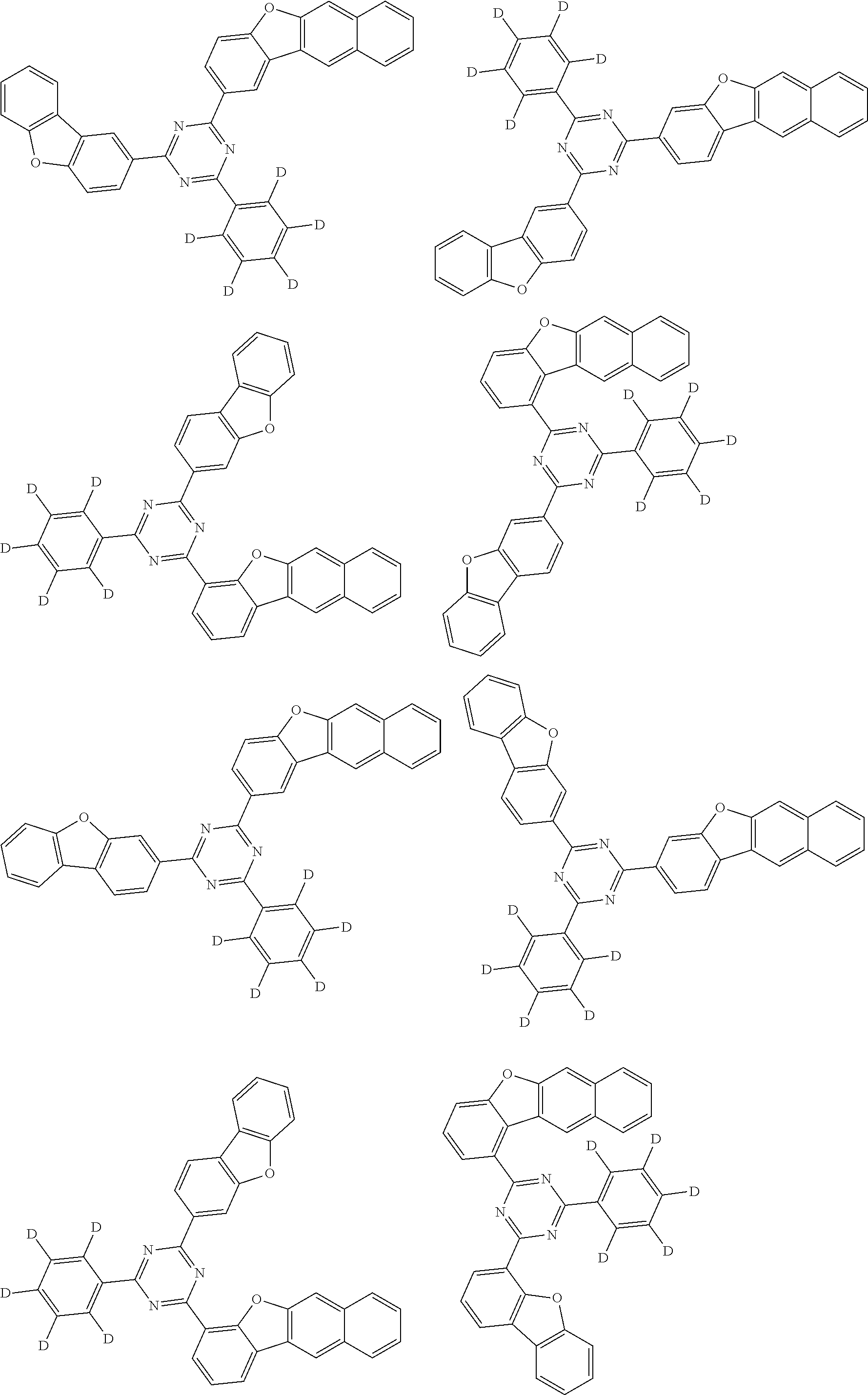

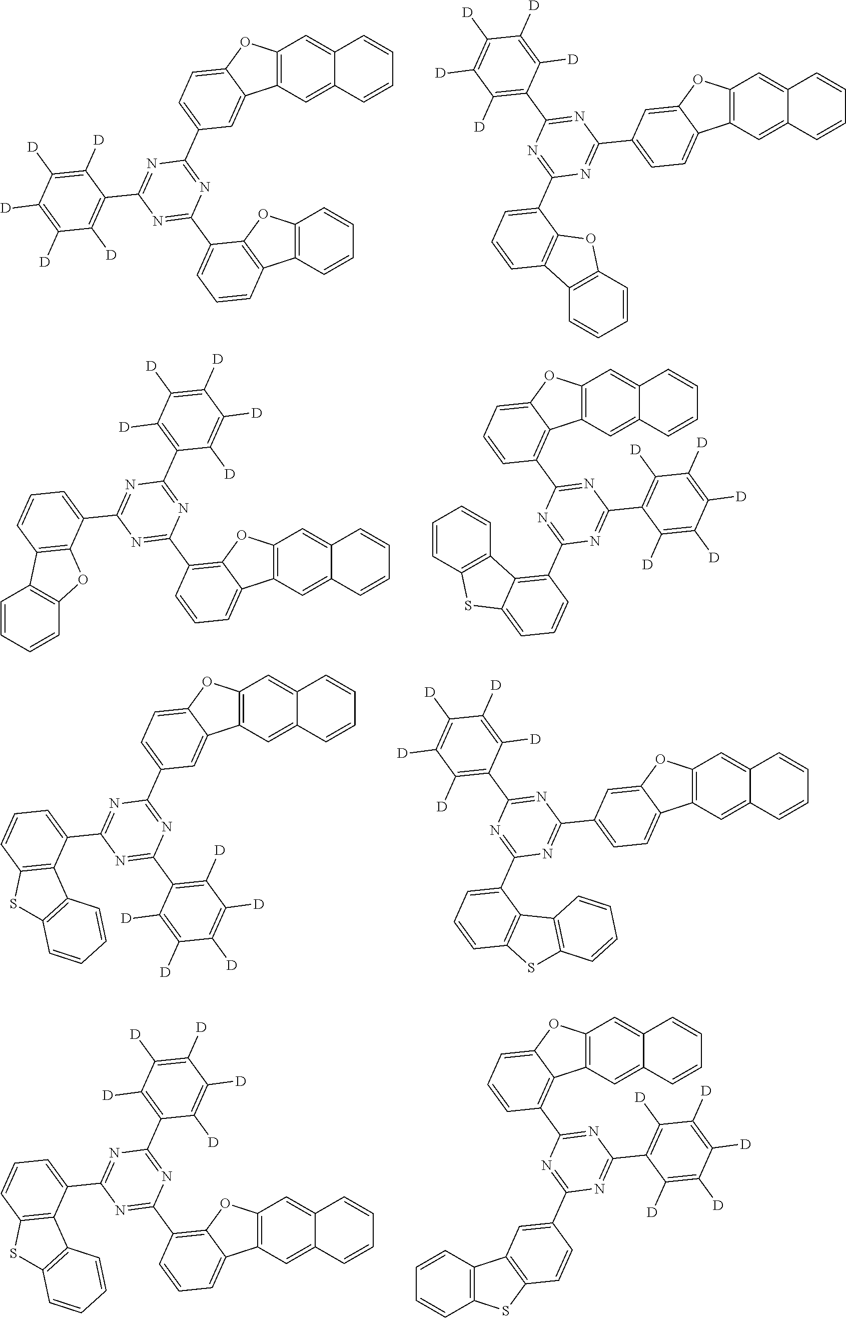

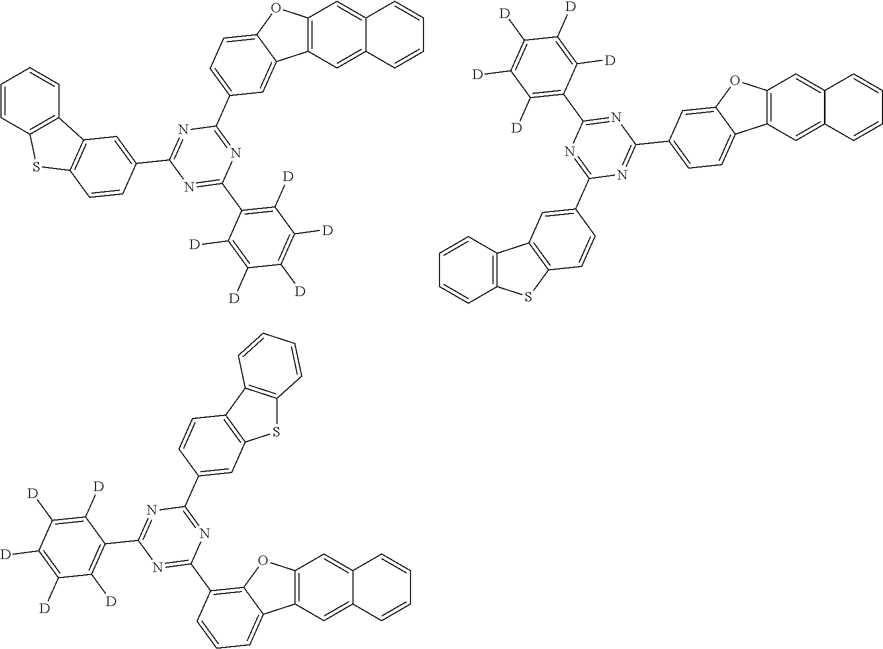

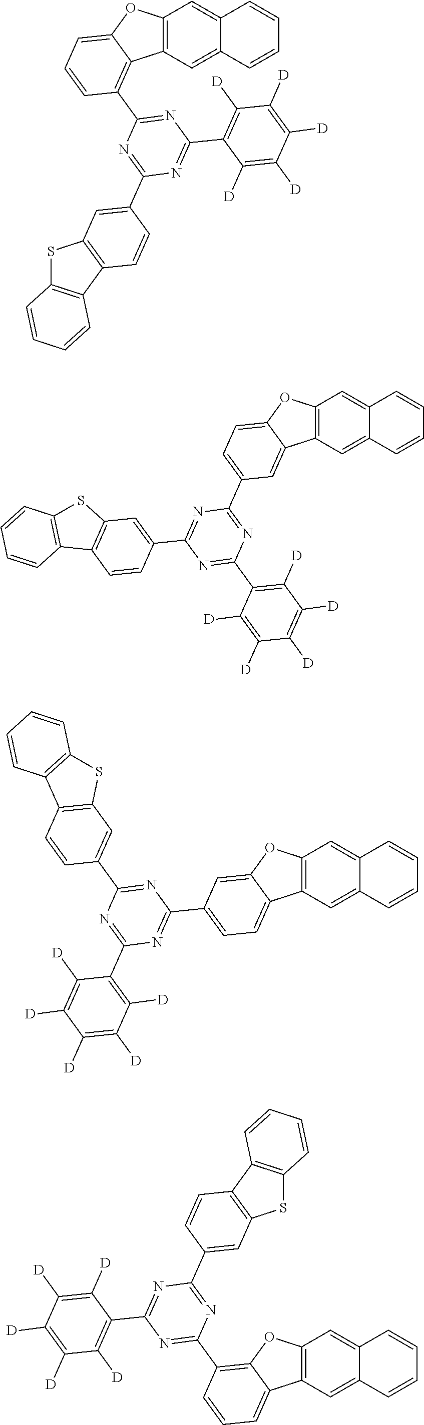 [SMILES: [2H]c1c([2H])c([2H])c(-c2nc(-c3ccc4c(c3)sc3ccccc34)nc(-c3cccc4oc5cc6ccccc6cc5c34)n2)c([2H])c1[2H] ; [2H]c1c([2H])c([2H])c(-c2nc(-c3ccc4c(c3)sc3ccccc34)nc(-c3ccc4oc5cc6ccccc6cc5c4c3)n2)c([2H])c1[2H] ; [2H]c1c([2H])c([2H])c(-c2nc(-c3ccc4c(c3)oc3cc5ccccc5cc34)nc(-c3ccc4c(c3)sc3ccccc34)n2)c([2H])c1[2H] ; [2H]c1c([2H])c([2H])c(-c2nc(-c3ccc4c(c3)sc3ccccc34)nc(-c3cccc4c3oc3cc5ccccc5cc34)n2)c([2H])c1[2H]]

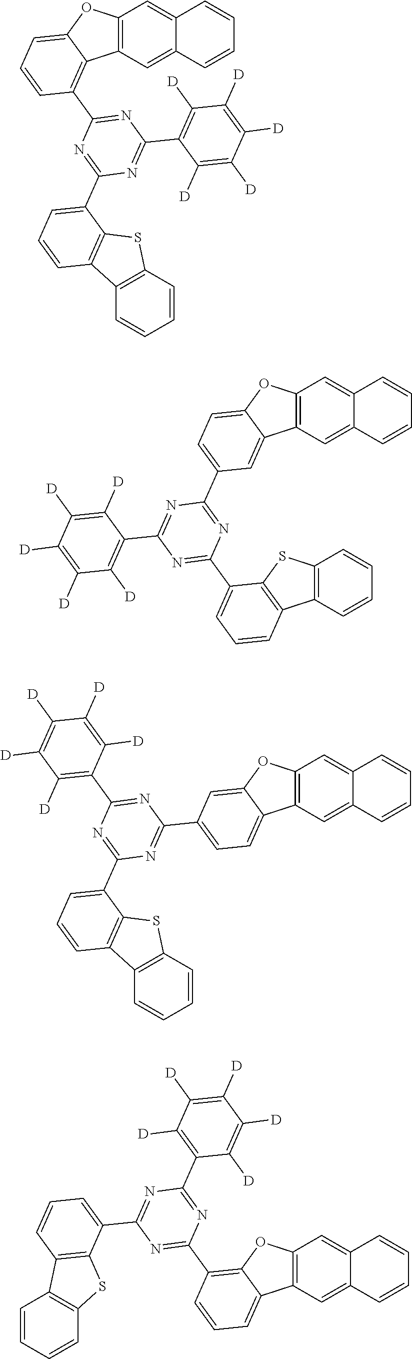 [SMILES: [2H]c1c([2H])c([2H])c(-c2nc(-c3cccc4c3sc3ccccc34)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cccc4c3sc3ccccc34)n2)c([2H])c1[2H] ; [2H]c1c([2H])c([2H])c(-c2nc(-c3ccc4c(c3)oc3cc5ccccc5cc34)nc(-c3cccc4c3sc3ccccc34)n2)c([2H])c1[2H] ; [2H]c1c([2H])c([2H])c(-c2nc(-c3cccc4c3oc3cc5ccccc5cc34)nc(-c3cccc4c3sc3ccccc34)n2)c([2H])c1[2H]]

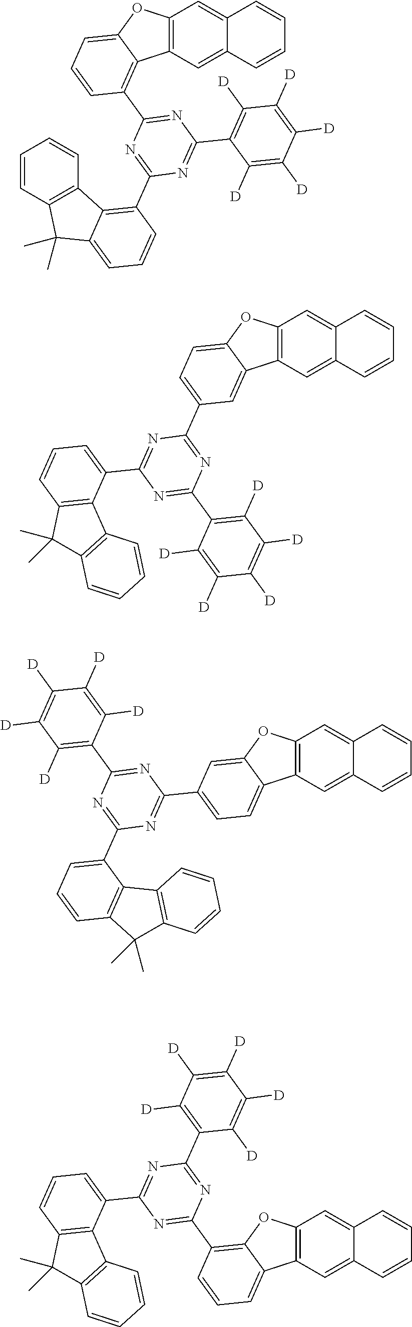 [SMILES: [2H]c1c([2H])c([2H])c(-c2nc(-c3cccc4c3-c3ccccc3C4(C)C)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cccc4c3-c3ccccc3C4(C)C)n2)c([2H])c1[2H] ; [2H]c1c([2H])c([2H])c(-c2nc(-c3ccc4c(c3)oc3cc5ccccc5cc34)nc(-c3cccc4c3-c3ccccc3C4(C)C)n2)c([2H])c1[2H] ; [2H]c1c([2H])c([2H])c(-c2nc(-c3cccc4c3-c3ccccc3C4(C)C)nc(-c3cccc4c3oc3cc5ccccc5cc34)n2)c([2H])c1[2H]]

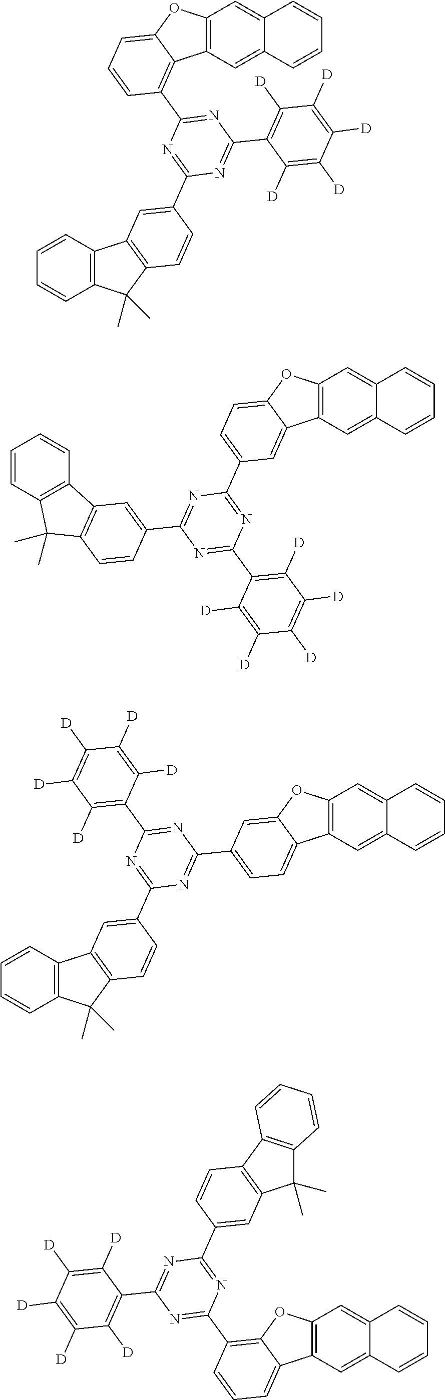 [SMILES: [2H]c1c([2H])c([2H])c(-c2nc(-c3ccc4c(c3)-c3ccccc3C4(C)C)nc(-c3cccc4oc5cc6ccccc6cc5c34)n2)c([2H])c1[2H] ; [2H]c1c([2H])c([2H])c(-c2nc(-c3ccc4c(c3)-c3ccccc3C4(C)C)nc(-c3ccc4oc5cc6ccccc6cc5c4c3)n2)c([2H])c1[2H] ; [2H]c1c([2H])c([2H])c(-c2nc(-c3ccc4c(c3)-c3ccccc3C4(C)C)nc(-c3ccc4c(c3)oc3cc5ccccc5cc34)n2)c([2H])c1[2H] ; [2H]c1c([2H])c([2H])c(-c2nc(-c3ccc4c(c3)C(C)(C)c3ccccc3-4)nc(-c3cccc4c3oc3cc5ccccc5cc34)n2)c([2H])c1[2H]]

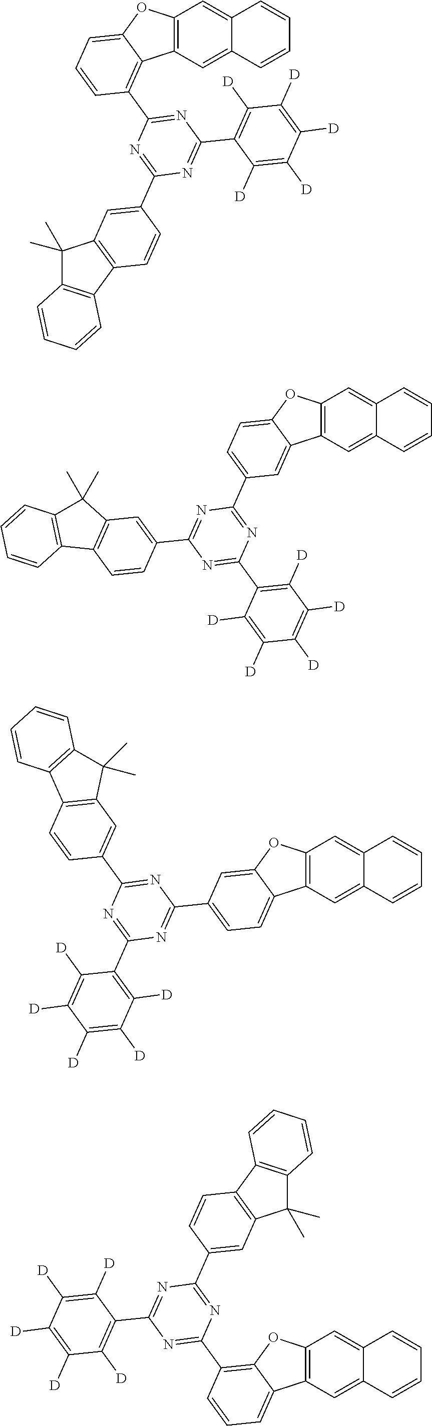 [SMILES: [2H]c1c([2H])c([2H])c(-c2nc(-c3ccc4c(c3)C(C)(C)c3ccccc3-4)nc(-c3cccc4oc5cc6ccccc6cc5c34)n2)c([2H])c1[2H] ; [2H]c1c([2H])c([2H])c(-c2nc(-c3ccc4c(c3)C(C)(C)c3ccccc3-4)nc(-c3ccc4oc5cc6ccccc6cc5c4c3)n2)c([2H])c1[2H] ; [2H]c1c([2H])c([2H])c(-c2nc(-c3ccc4c(c3)C(C)(C)c3ccccc3-4)nc(-c3ccc4c(c3)oc3cc5ccccc5cc34)n2)c([2H])c1[2H] ; [2H]c1c([2H])c([2H])c(-c2nc(-c3ccc4c(c3)C(C)(C)c3ccccc3-4)nc(-c3cccc4c3oc3cc5ccccc5cc34)n2)c([2H])c1[2H]]

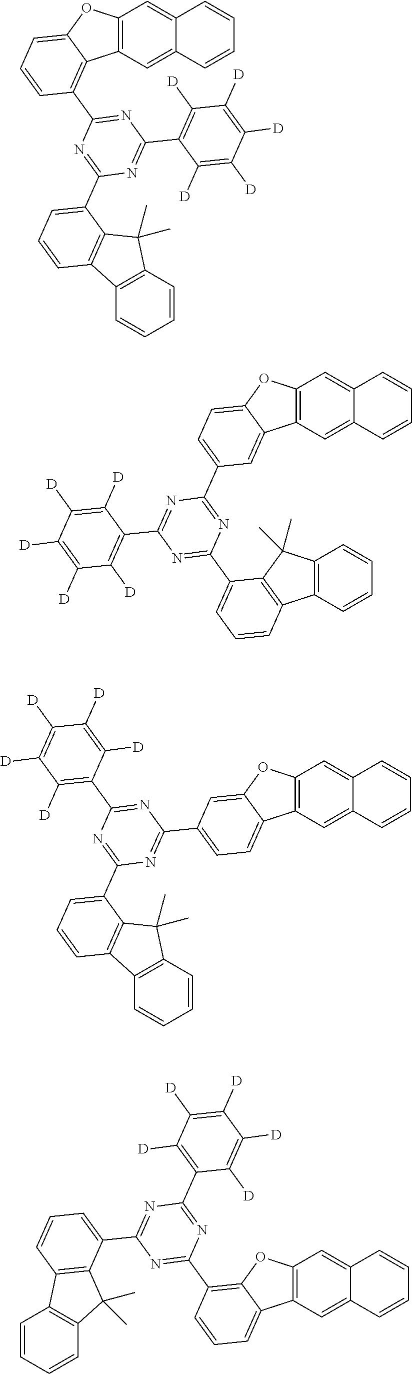 [SMILES: [2H]c1c([2H])c([2H])c(-c2nc(-c3cccc4c3C(C)(C)c3ccccc3-4)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cccc4c3C(C)(C)c3ccccc3-4)n2)c([2H])c1[2H] ; [2H]c1c([2H])c([2H])c(-c2nc(-c3ccc4c(c3)oc3cc5ccccc5cc34)nc(-c3cccc4c3C(C)(C)c3ccccc3-4)n2)c([2H])c1[2H] ; [2H]c1c([2H])c([2H])c(-c2nc(-c3cccc4c3C(C)(C)c3ccccc3-4)nc(-c3cccc4c3oc3cc5ccccc5cc34)n2)c([2H])c1[2H]]

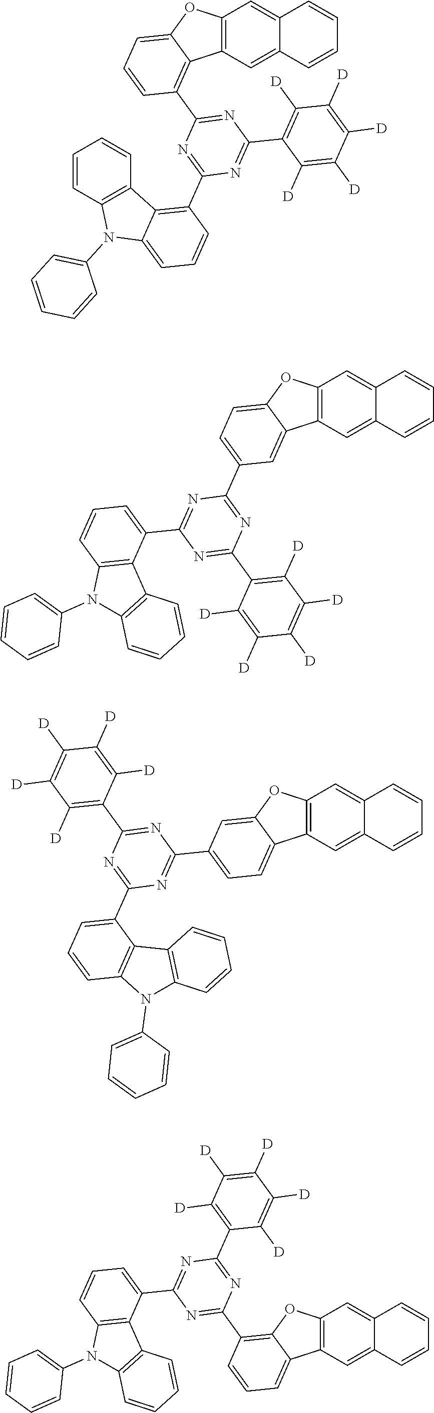 [SMILES: [2H]c1c([2H])c([2H])c(-c2nc(-c3cccc4oc5cc6ccccc6cc5c34)nc(-c3cccc4c3c3ccccc3n4-c3ccccc3)n2)c([2H])c1[2H] ; [2H]c1c([2H])c([2H])c(-c2nc(-c3ccc4oc5cc6ccccc6cc5c4c3)nc(-c3cccc4c3c3ccccc3n4-c3ccccc3)n2)c([2H])c1[2H] ; [2H]c1c([2H])c([2H])c(-c2nc(-c3ccc4c(c3)oc3cc5ccccc5cc34)nc(-c3cccc4c3c3ccccc3n4-c3ccccc3)n2)c([2H])c1[2H] ; [2H]c1c([2H])c([2H])c(-c2nc(-c3cccc4c3oc3cc5ccccc5cc34)nc(-c3cccc4c3c3ccccc3n4-c3ccccc3)n2)c([2H])c1[2H]]

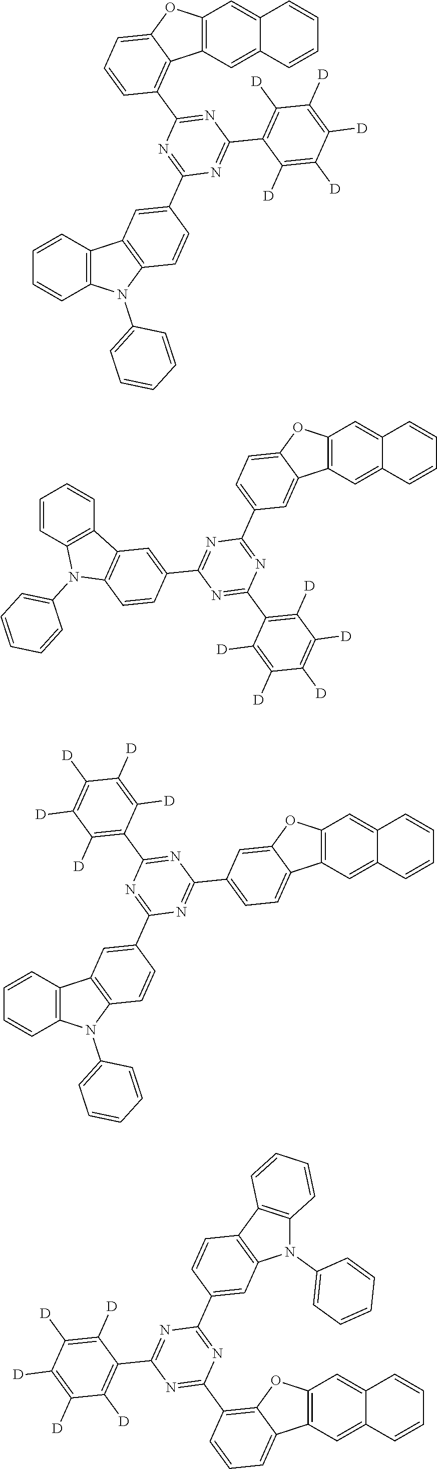 [SMILES: [2H]c1c([2H])c([2H])c(-c2nc(-c3ccc4c(c3)c3ccccc3n4-c3ccccc3)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3ccc4c(c3)c3ccccc3n4-c3ccccc3)n2)c([2H])c1[2H] ; [2H]c1c([2H])c([2H])c(-c2nc(-c3ccc4c(c3)oc3cc5ccccc5cc34)nc(-c3ccc4c(c3)c3ccccc3n4-c3ccccc3)n2)c([2H])c1[2H] ; [2H]c1c([2H])c([2H])c(-c2nc(-c3ccc4c5ccccc5n(-c5ccccc5)c4c3)nc(-c3cccc4c3oc3cc5ccccc5cc34)n2)c([2H])c1[2H]]

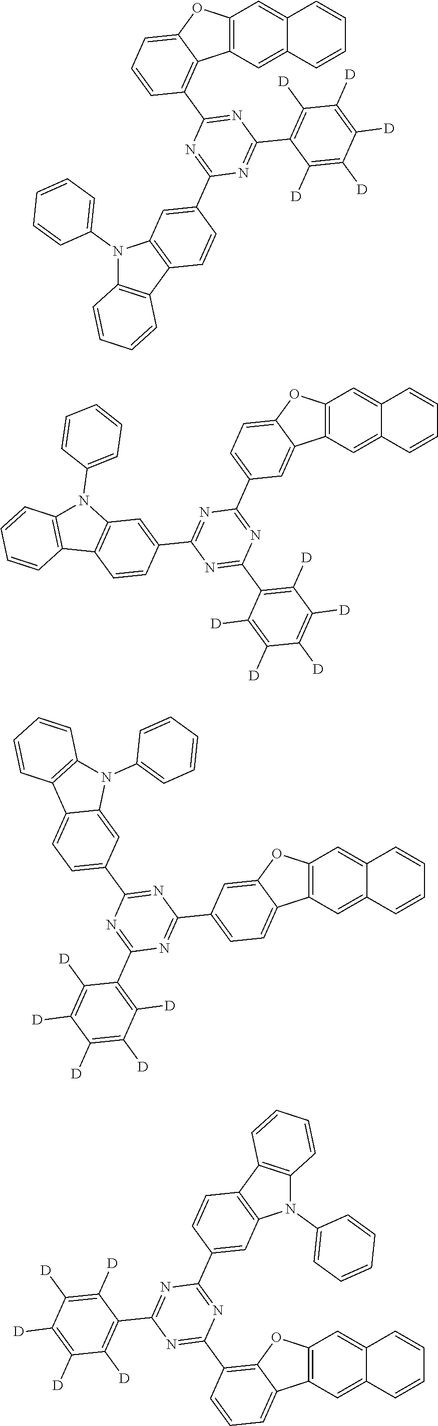 [SMILES: [2H]c1c([2H])c([2H])c(-c2nc(-c3ccc4c5ccccc5n(-c5ccccc5)c4c3)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3ccc4c5ccccc5n(-c5ccccc5)c4c3)n2)c([2H])c1[2H] ; [2H]c1c([2H])c([2H])c(-c2nc(-c3ccc4c(c3)oc3cc5ccccc5cc34)nc(-c3ccc4c5ccccc5n(-c5ccccc5)c4c3)n2)c([2H])c1[2H] ; [2H]c1c([2H])c([2H])c(-c2nc(-c3ccc4c5ccccc5n(-c5ccccc5)c4c3)nc(-c3cccc4c3oc3cc5ccccc5cc34)n2)c([2H])c1[2H]]

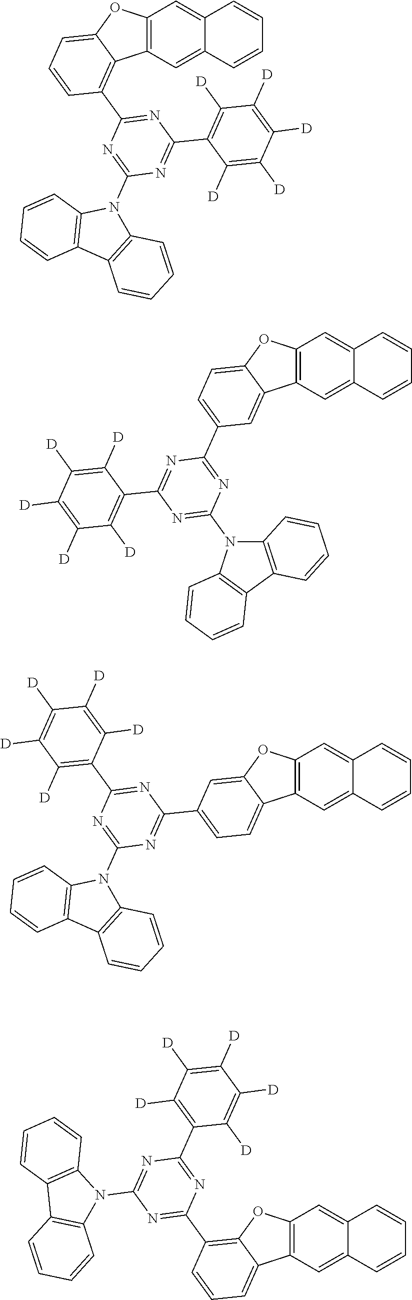 [SMILES: [2H]c1c([2H])c([2H])c(-c2nc(-c3cccc4oc5cc6ccccc6cc5c34)nc(-n3c4ccccc4c4ccccc43)n2)c([2H])c1[2H] ; [2H]c1c([2H])c([2H])c(-c2nc(-c3ccc4oc5cc6ccccc6cc5c4c3)nc(-n3c4ccccc4c4ccccc43)n2)c([2H])c1[2H] ; [2H]c1c([2H])c([2H])c(-c2nc(-c3ccc4c(c3)oc3cc5ccccc5cc34)nc(-n3c4ccccc4c4ccccc43)n2)c([2H])c1[2H] ; [2H]c1c([2H])c([2H])c(-c2nc(-c3cccc4c3oc3cc5ccccc5cc34)nc(-n3c4ccccc4c4ccccc43)n2)c([2H])c1[2H]]

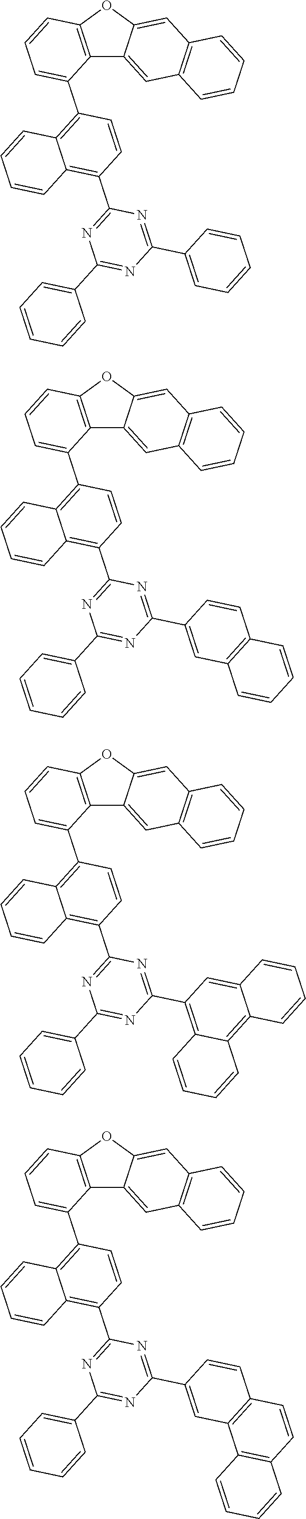

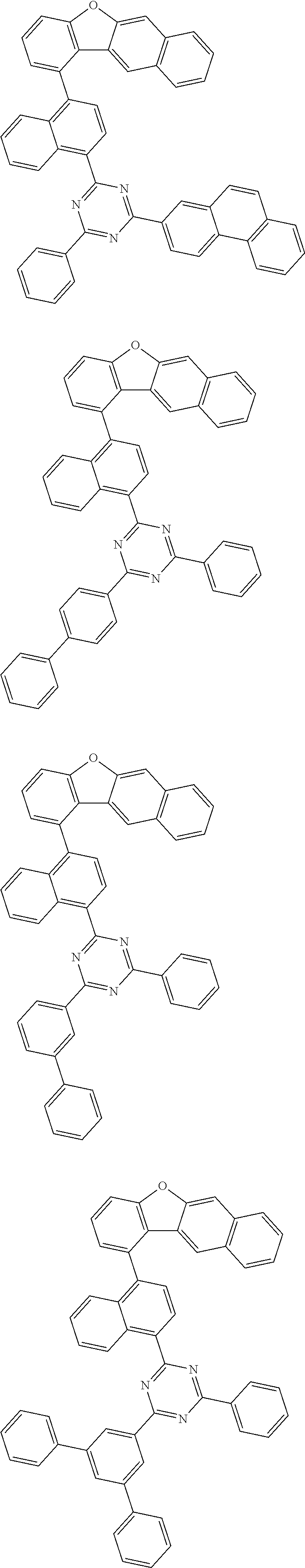 [SMILES: c1ccc(-c2nc(-c3ccc4c(ccc5ccccc54)c3)nc(-c3ccc(-c4cccc5oc6cc7ccccc7cc6c45)c4ccccc34)n2)cc1 ; c1ccc(-c2ccc(-c3nc(-c4ccccc4)nc(-c4ccc(-c5cccc6oc7cc8ccccc8cc7c56)c5ccccc45)n3)cc2)cc1 ; c1ccc(-c2cccc(-c3nc(-c4ccccc4)nc(-c4ccc(-c5cccc6oc7cc8ccccc8cc7c56)c5ccccc45)n3)c2)cc1 ; c1ccc(-c2cc(-c3ccccc3)cc(-c3nc(-c4ccccc4)nc(-c4ccc(-c5cccc6oc7cc8ccccc8cc7c56)c5ccccc45)n3)c2)cc1]

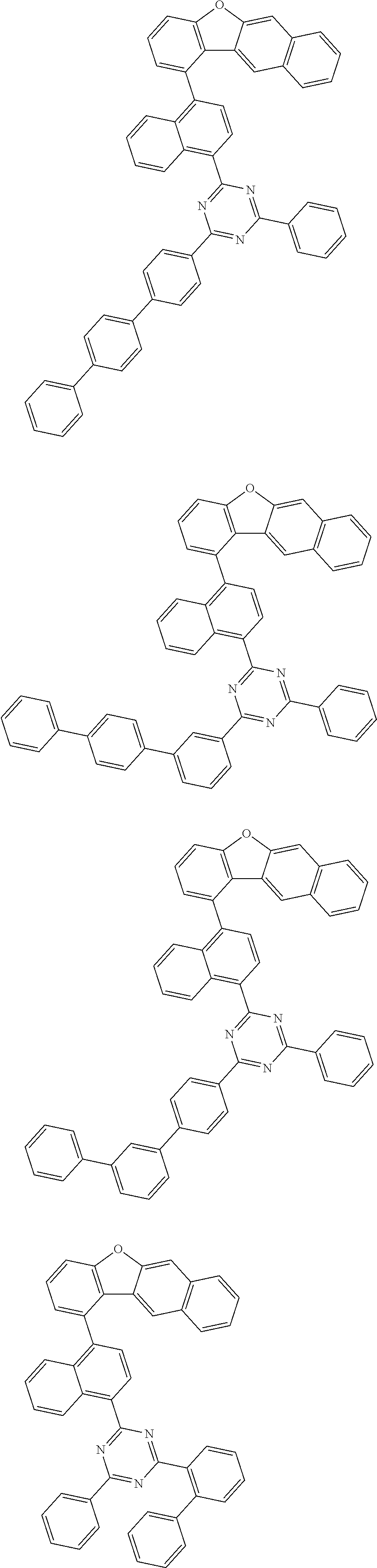 [SMILES: c1ccc(-c2ccc(-c3ccc(-c4nc(-c5ccccc5)nc(-c5ccc(-c6cccc7oc8cc9ccccc9cc8c67)c6ccccc56)n4)cc3)cc2)cc1 ; c1ccc(-c2ccc(-c3cccc(-c4nc(-c5ccccc5)nc(-c5ccc(-c6cccc7oc8cc9ccccc9cc8c67)c6ccccc56)n4)c3)cc2)cc1 ; c1ccc(-c2cccc(-c3ccc(-c4nc(-c5ccccc5)nc(-c5ccc(-c6cccc7oc8cc9ccccc9cc8c67)c6ccccc56)n4)cc3)c2)cc1 ; c1ccc(-c2nc(-c3ccccc3-c3ccccc3)nc(-c3ccc(-c4cccc5oc6cc7ccccc7cc6c45)c4ccccc34)n2)cc1]

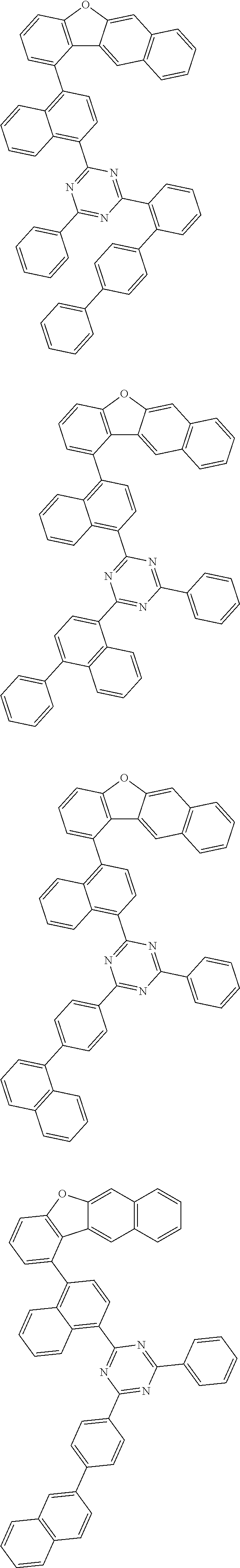 [SMILES: c1ccc(-c2ccc(-c3ccccc3-c3nc(-c4ccccc4)nc(-c4ccc(-c5cccc6oc7cc8ccccc8cc7c56)c5ccccc45)n3)cc2)cc1 ; c1ccc(-c2nc(-c3ccc(-c4ccccc4)c4ccccc34)nc(-c3ccc(-c4cccc5oc6cc7ccccc7cc6c45)c4ccccc34)n2)cc1 ; c1ccc(-c2nc(-c3ccc(-c4cccc5ccccc45)cc3)nc(-c3ccc(-c4cccc5oc6cc7ccccc7cc6c45)c4ccccc34)n2)cc1 ; c1ccc(-c2nc(-c3ccc(-c4ccc5ccccc5c4)cc3)nc(-c3ccc(-c4cccc5oc6cc7ccccc7cc6c45)c4ccccc34)n2)cc1]

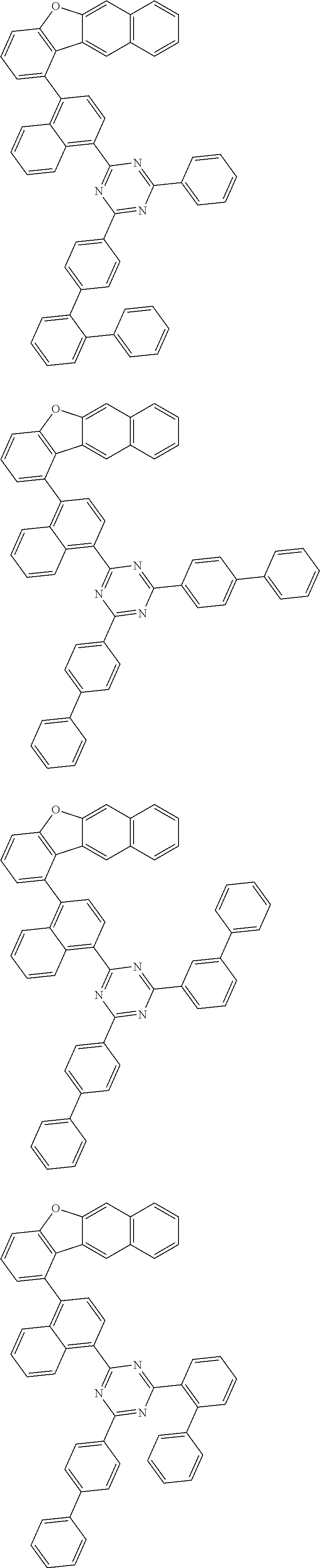 [SMILES: c1ccc(-c2nc(-c3ccc(-c4ccccc4-c4ccccc4)cc3)nc(-c3ccc(-c4cccc5oc6cc7ccccc7cc6c45)c4ccccc34)n2)cc1 ; c1ccc(-c2ccc(-c3nc(-c4ccc(-c5ccccc5)cc4)nc(-c4ccc(-c5cccc6oc7cc8ccccc8cc7c56)c5ccccc45)n3)cc2)cc1 ; c1ccc(-c2ccc(-c3nc(-c4cccc(-c5ccccc5)c4)nc(-c4ccc(-c5cccc6oc7cc8ccccc8cc7c56)c5ccccc45)n3)cc2)cc1 ; c1ccc(-c2ccc(-c3nc(-c4ccccc4-c4ccccc4)nc(-c4ccc(-c5cccc6oc7cc8ccccc8cc7c56)c5ccccc45)n3)cc2)cc1]

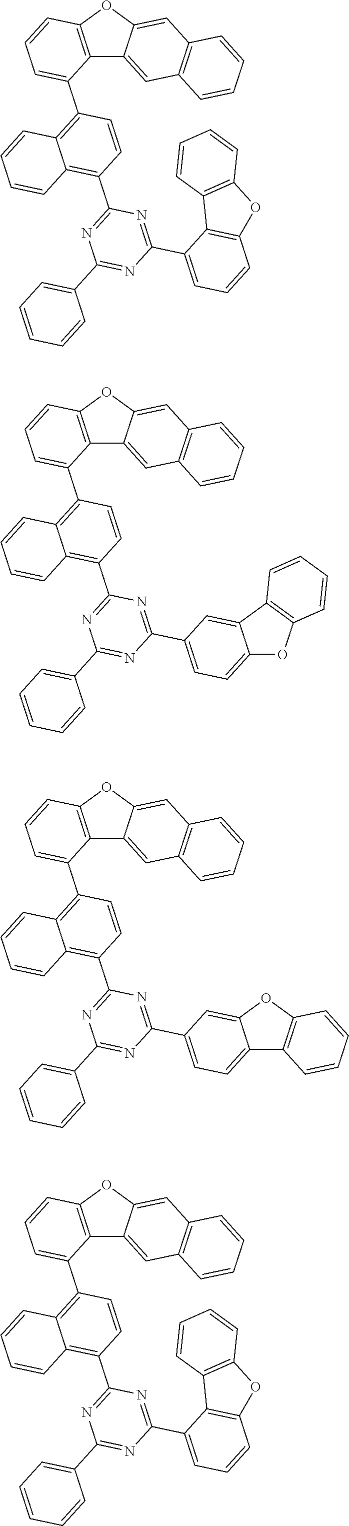 [SMILES: c1ccc(-c2nc(-c3ccc(-c4cccc5oc6cc7ccccc7cc6c45)c4ccccc34)nc(-c3cccc4oc5ccccc5c34)n2)cc1 ; c1ccc(-c2nc(-c3ccc4oc5ccccc5c4c3)nc(-c3ccc(-c4cccc5oc6cc7ccccc7cc6c45)c4ccccc34)n2)cc1 ; c1ccc(-c2nc(-c3ccc4c(c3)oc3ccccc34)nc(-c3ccc(-c4cccc5oc6cc7ccccc7cc6c45)c4ccccc34)n2)cc1 ; c1ccc(-c2nc(-c3ccc(-c4cccc5oc6cc7ccccc7cc6c45)c4ccccc34)nc(-c3cccc4oc5ccccc5c34)n2)cc1]

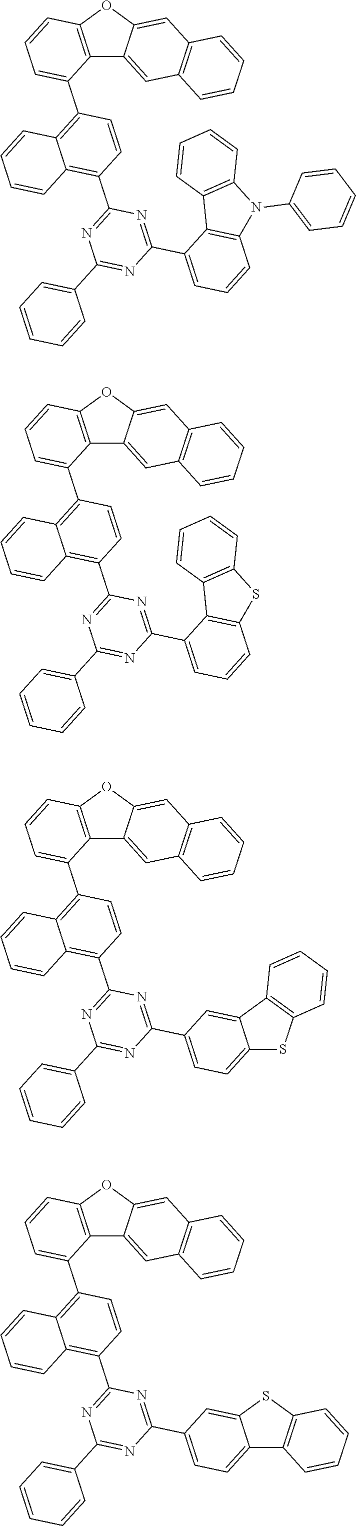 [SMILES: c1ccc(-c2nc(-c3ccc(-c4cccc5oc6cc7ccccc7cc6c45)c4ccccc34)nc(-c3cccc4c3c3ccccc3n4-c3ccccc3)n2)cc1 ; c1ccc(-c2nc(-c3ccc(-c4cccc5oc6cc7ccccc7cc6c45)c4ccccc34)nc(-c3cccc4sc5ccccc5c34)n2)cc1 ; c1ccc(-c2nc(-c3ccc4sc5ccccc5c4c3)nc(-c3ccc(-c4cccc5oc6cc7ccccc7cc6c45)c4ccccc34)n2)cc1 ; c1ccc(-c2nc(-c3ccc4c(c3)sc3ccccc34)nc(-c3ccc(-c4cccc5oc6cc7ccccc7cc6c45)c4ccccc34)n2)cc1]

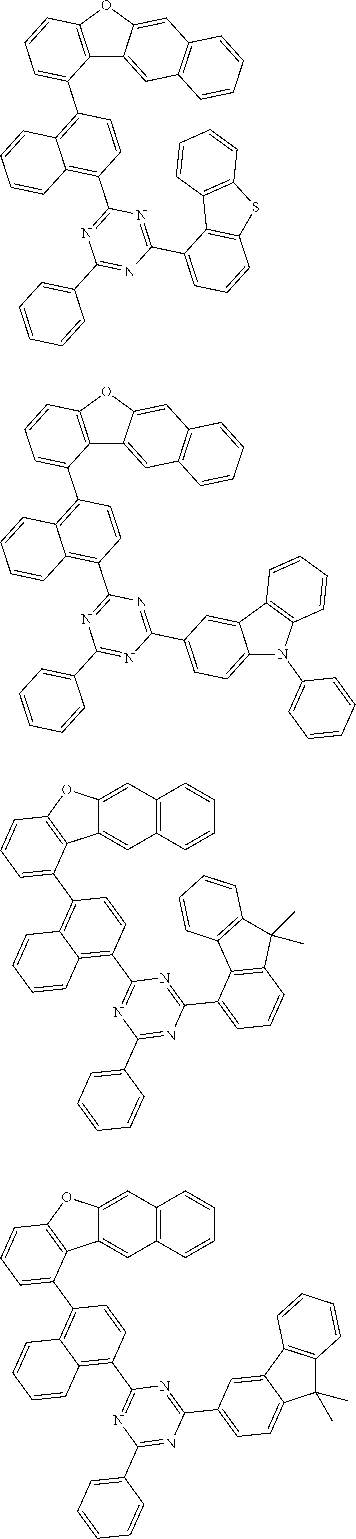 [SMILES: c1ccc(-c2nc(-c3ccc(-c4cccc5oc6cc7ccccc7cc6c45)c4ccccc34)nc(-c3cccc4sc5ccccc5c34)n2)cc1 ; c1ccc(-c2nc(-c3ccc4c(c3)c3ccccc3n4-c3ccccc3)nc(-c3ccc(-c4cccc5oc6cc7ccccc7cc6c45)c4ccccc34)n2)cc1 ; CC1(C)c2ccccc2-c2c(-c3nc(-c4ccccc4)nc(-c4ccc(-c5cccc6oc7cc8ccccc8cc7c56)c5ccccc45)n3)cccc21 ; CC1(C)c2ccccc2-c2cc(-c3nc(-c4ccccc4)nc(-c4ccc(-c5cccc6oc7cc8ccccc8cc7c56)c5ccccc45)n3)ccc21]

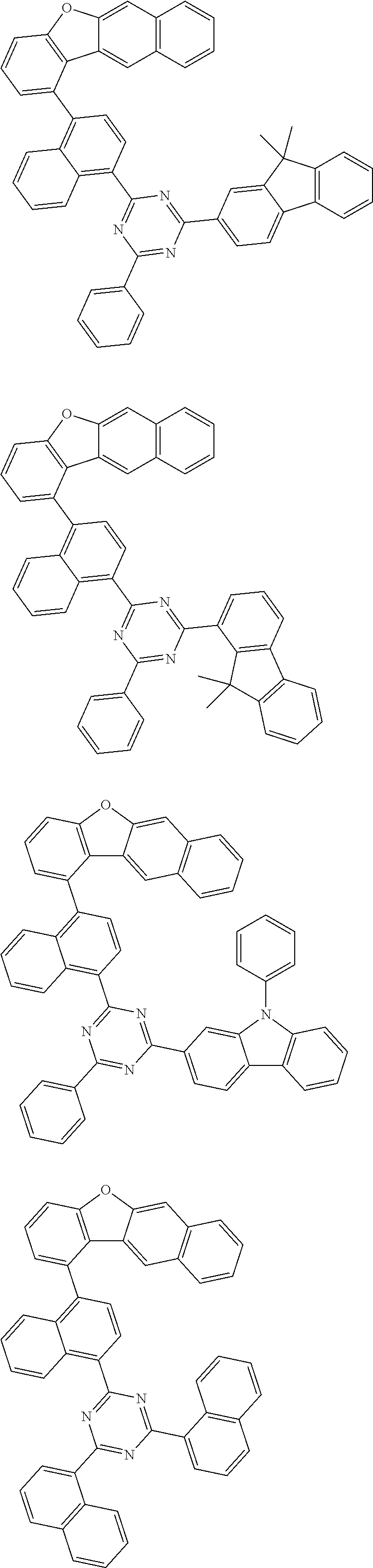 [SMILES: CC1(C)c2ccccc2-c2ccc(-c3nc(-c4ccccc4)nc(-c4ccc(-c5cccc6oc7cc8ccccc8cc7c56)c5ccccc45)n3)cc21 ; CC1(C)c2ccccc2-c2cccc(-c3nc(-c4ccccc4)nc(-c4ccc(-c5cccc6oc7cc8ccccc8cc7c56)c5ccccc45)n3)c21 ; c1ccc(-c2nc(-c3ccc4c5ccccc5n(-c5ccccc5)c4c3)nc(-c3ccc(-c4cccc5oc6cc7ccccc7cc6c45)c4ccccc34)n2)cc1 ; c1ccc2cc3c(cc2c1)oc1cccc(-c2ccc(-c4nc(-c5cccc6ccccc56)nc(-c5cccc6ccccc56)n4)c4ccccc24)c13]

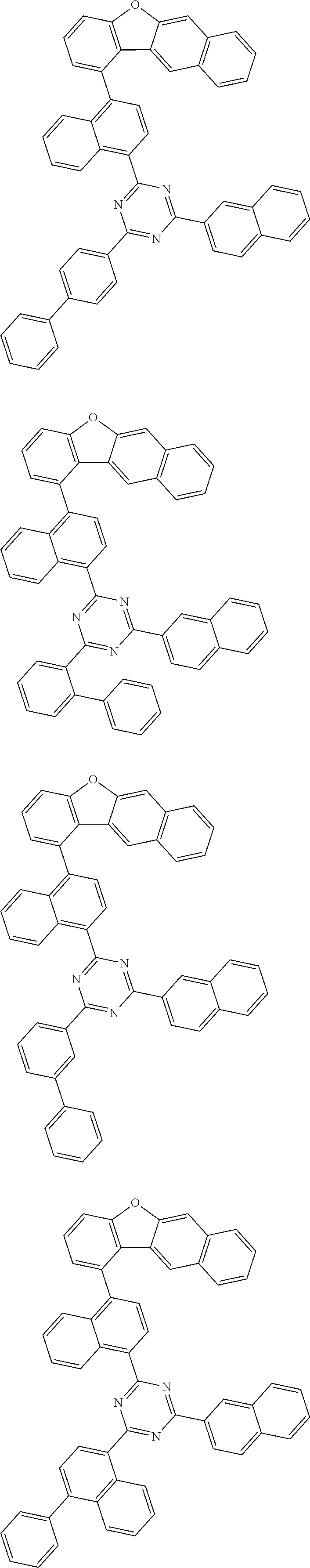 [SMILES: c1ccc(-c2ccc(-c3nc(-c4ccc5ccccc5c4)nc(-c4ccc(-c5cccc6oc7cc8ccccc8cc7c56)c5ccccc45)n3)cc2)cc1 ; c1ccc(-c2ccccc2-c2nc(-c3ccc4ccccc4c3)nc(-c3ccc(-c4cccc5oc6cc7ccccc7cc6c45)c4ccccc34)n2)cc1 ; c1ccc(-c2cccc(-c3nc(-c4ccc5ccccc5c4)nc(-c4ccc(-c5cccc6oc7cc8ccccc8cc7c56)c5ccccc45)n3)c2)cc1 ; c1ccc(-c2ccc(-c3nc(-c4ccc5ccccc5c4)nc(-c4ccc(-c5cccc6oc7cc8ccccc8cc7c56)c5ccccc45)n3)c3ccccc23)cc1]

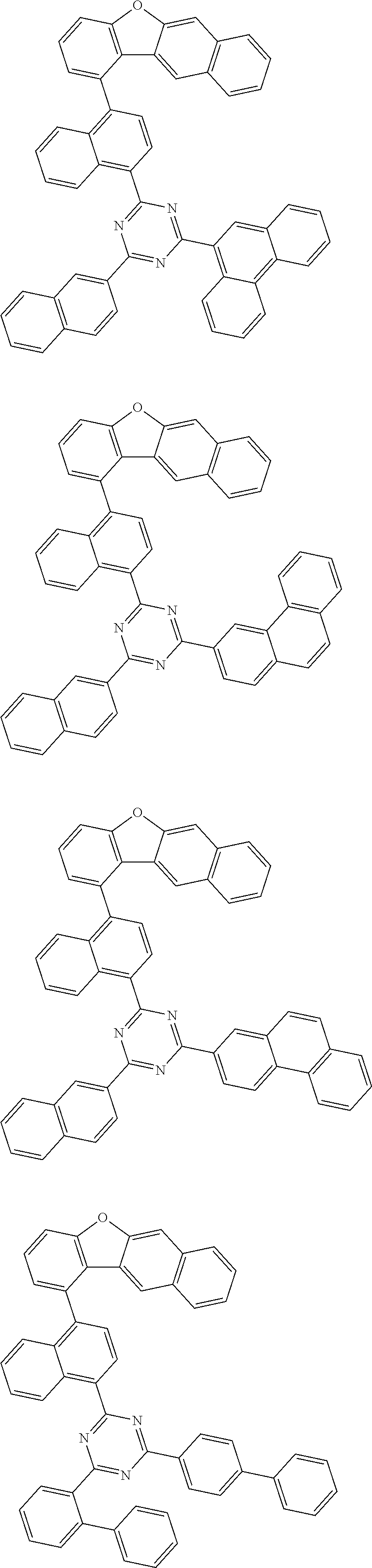 [SMILES: c1ccc2cc(-c3nc(-c4ccc(-c5cccc6oc7cc8ccccc8cc7c56)c5ccccc45)nc(-c4cc5ccccc5c5ccccc45)n3)ccc2c1 ; c1ccc2cc(-c3nc(-c4ccc5ccc6ccccc6c5c4)nc(-c4ccc(-c5cccc6oc7cc8ccccc8cc7c56)c5ccccc45)n3)ccc2c1 ; c1ccc2cc(-c3nc(-c4ccc5c(ccc6ccccc65)c4)nc(-c4ccc(-c5cccc6oc7cc8ccccc8cc7c56)c5ccccc45)n3)ccc2c1 ; c1ccc(-c2ccc(-c3nc(-c4ccccc4-c4ccccc4)nc(-c4ccc(-c5cccc6oc7cc8ccccc8cc7c56)c5ccccc45)n3)cc2)cc1]

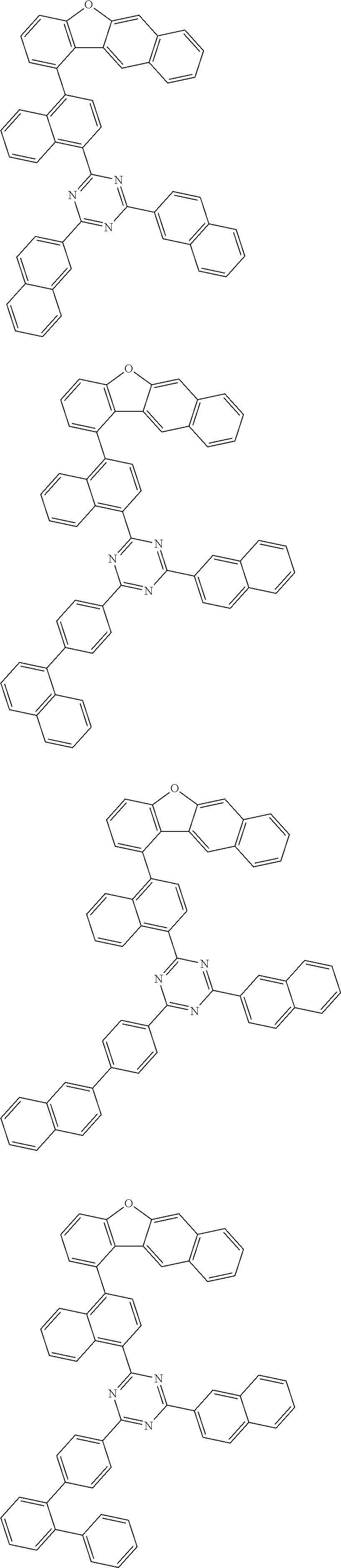 [SMILES: c1ccc2cc(-c3nc(-c4ccc5ccccc5c4)nc(-c4ccc(-c5cccc6oc7cc8ccccc8cc7c56)c5ccccc45)n3)ccc2c1 ; c1ccc2cc(-c3nc(-c4ccc(-c5cccc6ccccc56)cc4)nc(-c4ccc(-c5cccc6oc7cc8ccccc8cc7c56)c5ccccc45)n3)ccc2c1 ; c1ccc2cc(-c3ccc(-c4nc(-c5ccc6ccccc6c5)nc(-c5ccc(-c6cccc7oc8cc9ccccc9cc8c67)c6ccccc56)n4)cc3)ccc2c1 ; c1ccc(-c2ccccc2-c2ccc(-c3nc(-c4ccc5ccccc5c4)nc(-c4ccc(-c5cccc6oc7cc8ccccc8cc7c56)c5ccccc45)n3)cc2)cc1]

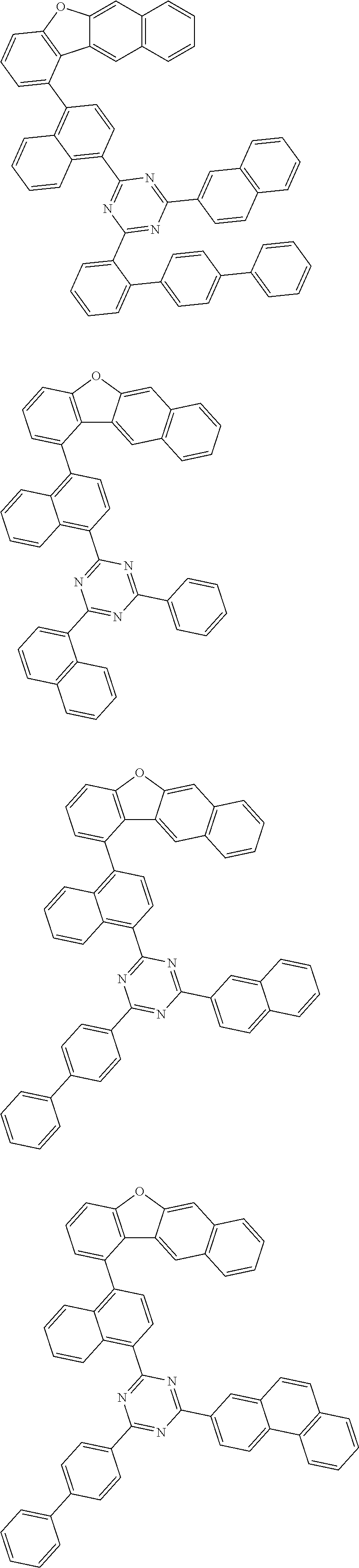 [SMILES: c1ccc(-c2ccc(-c3ccccc3-c3nc(-c4ccc5ccccc5c4)nc(-c4ccc(-c5cccc6oc7cc8ccccc8cc7c56)c5ccccc45)n3)cc2)cc1 ; c1ccc(-c2nc(-c3cccc4ccccc34)nc(-c3ccc(-c4cccc5oc6cc7ccccc7cc6c45)c4ccccc34)n2)cc1 ; c1ccc(-c2ccc(-c3nc(-c4ccc5ccccc5c4)nc(-c4ccc(-c5cccc6oc7cc8ccccc8cc7c56)c5ccccc45)n3)cc2)cc1 ; c1ccc(-c2ccc(-c3nc(-c4ccc5c(ccc6ccccc65)c4)nc(-c4ccc(-c5cccc6oc7cc8ccccc8cc7c56)c5ccccc45)n3)cc2)cc1]

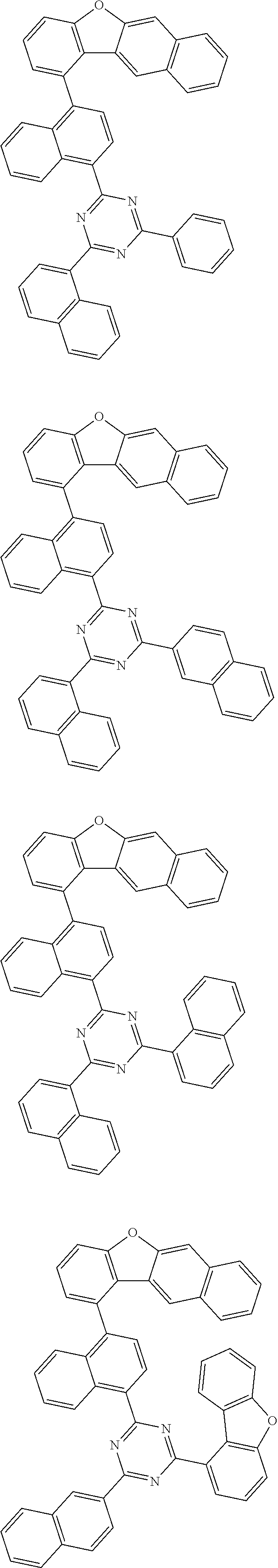 [SMILES: c1ccc(-c2nc(-c3cccc4ccccc34)nc(-c3ccc(-c4cccc5oc6cc7ccccc7cc6c45)c4ccccc34)n2)cc1 ; c1ccc2cc(-c3nc(-c4cccc5ccccc45)nc(-c4ccc(-c5cccc6oc7cc8ccccc8cc7c56)c5ccccc45)n3)ccc2c1 ; c1ccc2cc3c(cc2c1)oc1cccc(-c2ccc(-c4nc(-c5cccc6ccccc56)nc(-c5cccc6ccccc56)n4)c4ccccc24)c13 ; c1ccc2cc(-c3nc(-c4ccc(-c5cccc6oc7cc8ccccc8cc7c56)c5ccccc45)nc(-c4cccc5oc6ccccc6c45)n3)ccc2c1]

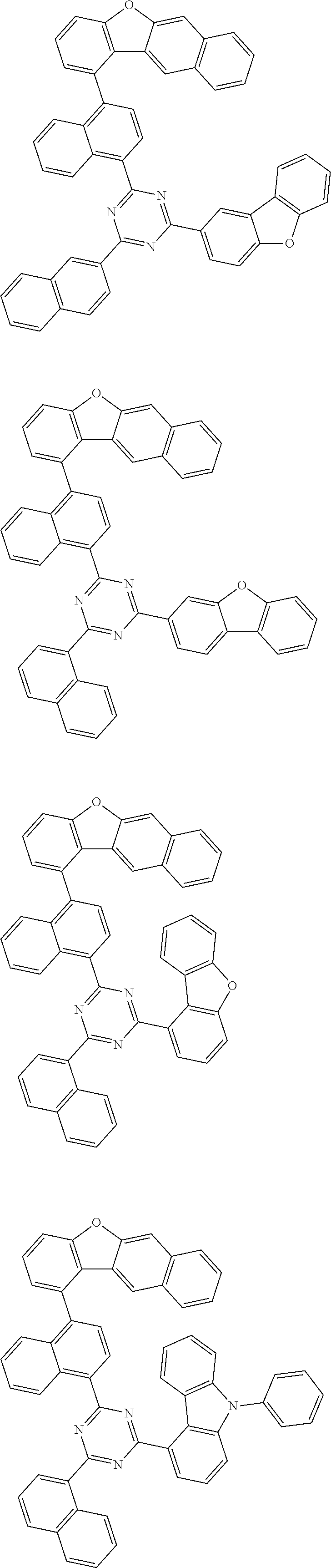 [SMILES: c1ccc2cc(-c3nc(-c4ccc5oc6ccccc6c5c4)nc(-c4ccc(-c5cccc6oc7cc8ccccc8cc7c56)c5ccccc45)n3)ccc2c1 ; c1ccc2cc3c(cc2c1)oc1cccc(-c2ccc(-c4nc(-c5ccc6c(c5)oc5ccccc56)nc(-c5cccc6ccccc56)n4)c4ccccc24)c13 ; c1ccc2cc3c(cc2c1)oc1cccc(-c2ccc(-c4nc(-c5cccc6ccccc56)nc(-c5cccc6oc7ccccc7c56)n4)c4ccccc24)c13 ; c1ccc(-n2c3ccccc3c3c(-c4nc(-c5cccc6ccccc56)nc(-c5ccc(-c6cccc7oc8cc9ccccc9cc8c67)c6ccccc56)n4)cccc32)cc1]

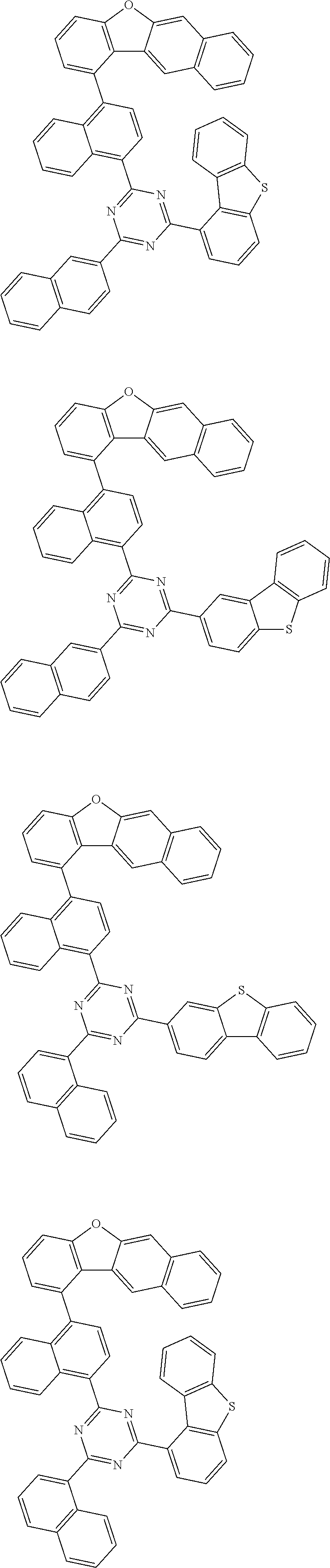 [SMILES: c1ccc2cc(-c3nc(-c4ccc(-c5cccc6oc7cc8ccccc8cc7c56)c5ccccc45)nc(-c4cccc5sc6ccccc6c45)n3)ccc2c1 ; c1ccc2cc(-c3nc(-c4ccc5sc6ccccc6c5c4)nc(-c4ccc(-c5cccc6oc7cc8ccccc8cc7c56)c5ccccc45)n3)ccc2c1 ; c1ccc2cc3c(cc2c1)oc1cccc(-c2ccc(-c4nc(-c5ccc6c(c5)sc5ccccc56)nc(-c5cccc6ccccc56)n4)c4ccccc24)c13 ; c1ccc2cc3c(cc2c1)oc1cccc(-c2ccc(-c4nc(-c5cccc6ccccc56)nc(-c5cccc6sc7ccccc7c56)n4)c4ccccc24)c13]

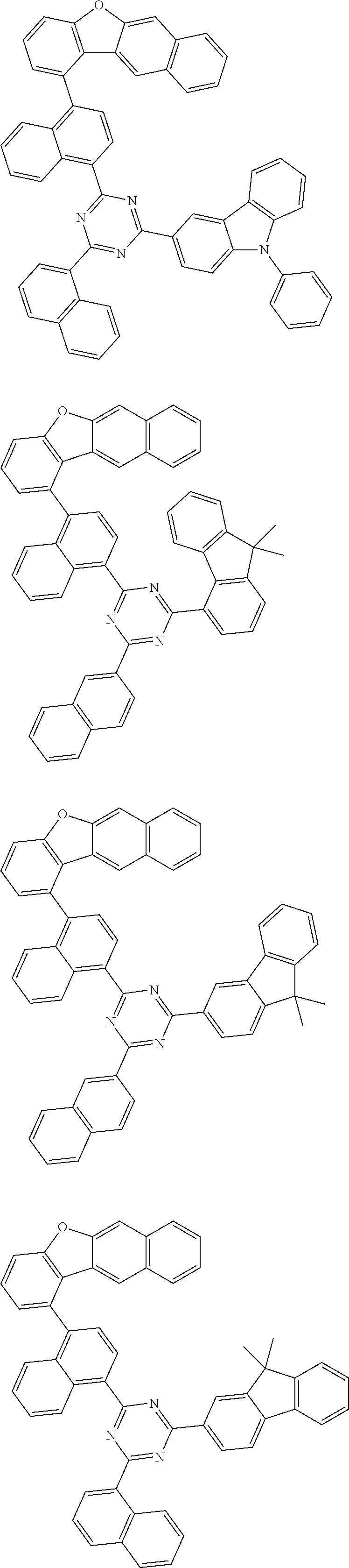 [SMILES: c1ccc(-n2c3ccccc3c3cc(-c4nc(-c5cccc6ccccc56)nc(-c5ccc(-c6cccc7oc8cc9ccccc9cc8c67)c6ccccc56)n4)ccc32)cc1 ; CC1(C)c2ccccc2-c2c(-c3nc(-c4ccc5ccccc5c4)nc(-c4ccc(-c5cccc6oc7cc8ccccc8cc7c56)c5ccccc45)n3)cccc21 ; CC1(C)c2ccccc2-c2cc(-c3nc(-c4ccc5ccccc5c4)nc(-c4ccc(-c5cccc6oc7cc8ccccc8cc7c56)c5ccccc45)n3)ccc21 ; CC1(C)c2ccccc2-c2ccc(-c3nc(-c4cccc5ccccc45)nc(-c4ccc(-c5cccc6oc7cc8ccccc8cc7c56)c5ccccc45)n3)cc21]

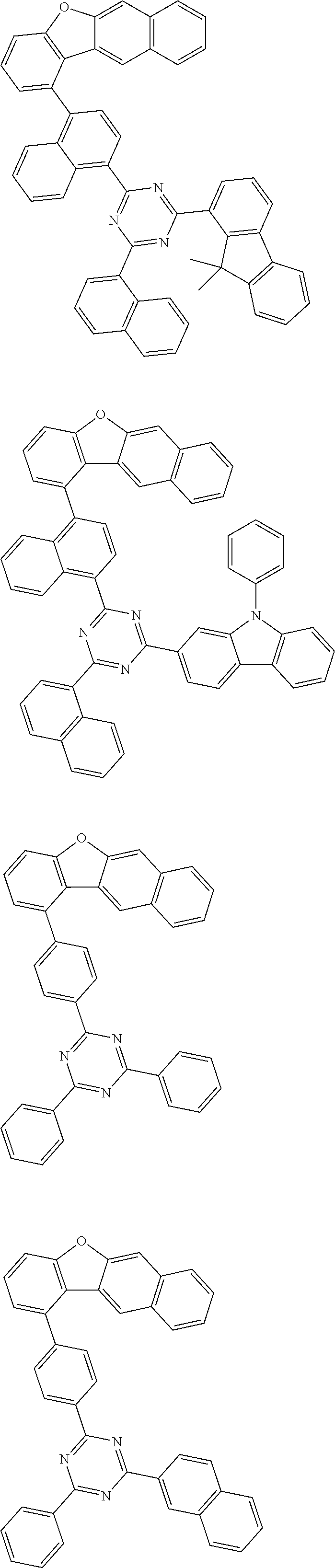 [SMILES: CC1(C)c2ccccc2-c2cccc(-c3nc(-c4cccc5ccccc45)nc(-c4ccc(-c5cccc6oc7cc8ccccc8cc7c56)c5ccccc45)n3)c21 ; c1ccc(-n2c3ccccc3c3ccc(-c4nc(-c5cccc6ccccc56)nc(-c5ccc(-c6cccc7oc8cc9ccccc9cc8c67)c6ccccc56)n4)cc32)cc1 ; c1ccc(-c2nc(-c3ccccc3)nc(-c3ccc(-c4cccc5oc6cc7ccccc7cc6c45)cc3)n2)cc1 ; c1ccc(-c2nc(-c3ccc(-c4cccc5oc6cc7ccccc7cc6c45)cc3)nc(-c3ccc4ccccc4c3)n2)cc1]

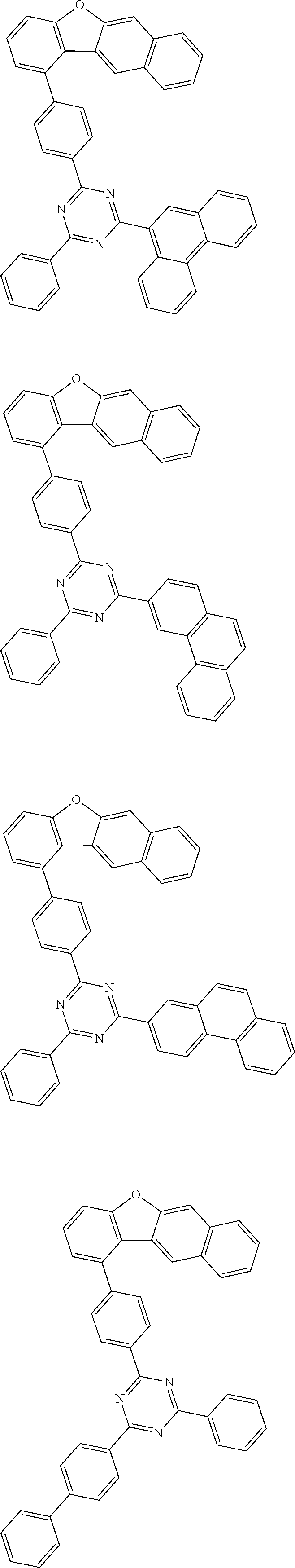 [SMILES: c1ccc(-c2nc(-c3ccc(-c4cccc5oc6cc7ccccc7cc6c45)cc3)nc(-c3cc4ccccc4c4ccccc34)n2)cc1 ; c1ccc(-c2nc(-c3ccc(-c4cccc5oc6cc7ccccc7cc6c45)cc3)nc(-c3ccc4ccc5ccccc5c4c3)n2)cc1 ; c1ccc(-c2nc(-c3ccc(-c4cccc5oc6cc7ccccc7cc6c45)cc3)nc(-c3ccc4c(ccc5ccccc54)c3)n2)cc1 ; c1ccc(-c2ccc(-c3nc(-c4ccccc4)nc(-c4ccc(-c5cccc6oc7cc8ccccc8cc7c56)cc4)n3)cc2)cc1]

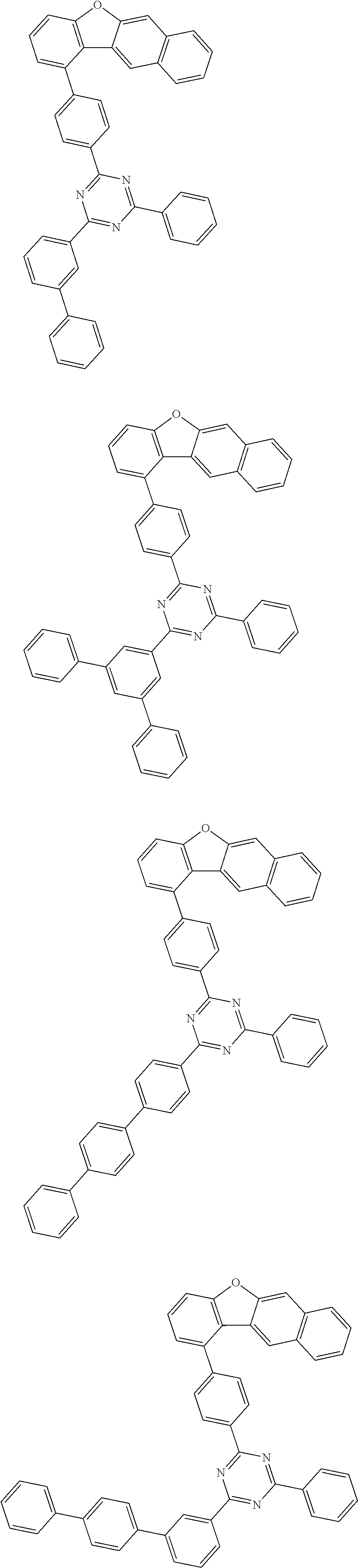 [SMILES: c1ccc(-c2cccc(-c3nc(-c4ccccc4)nc(-c4ccc(-c5cccc6oc7cc8ccccc8cc7c56)cc4)n3)c2)cc1 ; c1ccc(-c2cc(-c3ccccc3)cc(-c3nc(-c4ccccc4)nc(-c4ccc(-c5cccc6oc7cc8ccccc8cc7c56)cc4)n3)c2)cc1 ; c1ccc(-c2ccc(-c3ccc(-c4nc(-c5ccccc5)nc(-c5ccc(-c6cccc7oc8cc9ccccc9cc8c67)cc5)n4)cc3)cc2)cc1 ; c1ccc(-c2ccc(-c3cccc(-c4nc(-c5ccccc5)nc(-c5ccc(-c6cccc7oc8cc9ccccc9cc8c67)cc5)n4)c3)cc2)cc1]

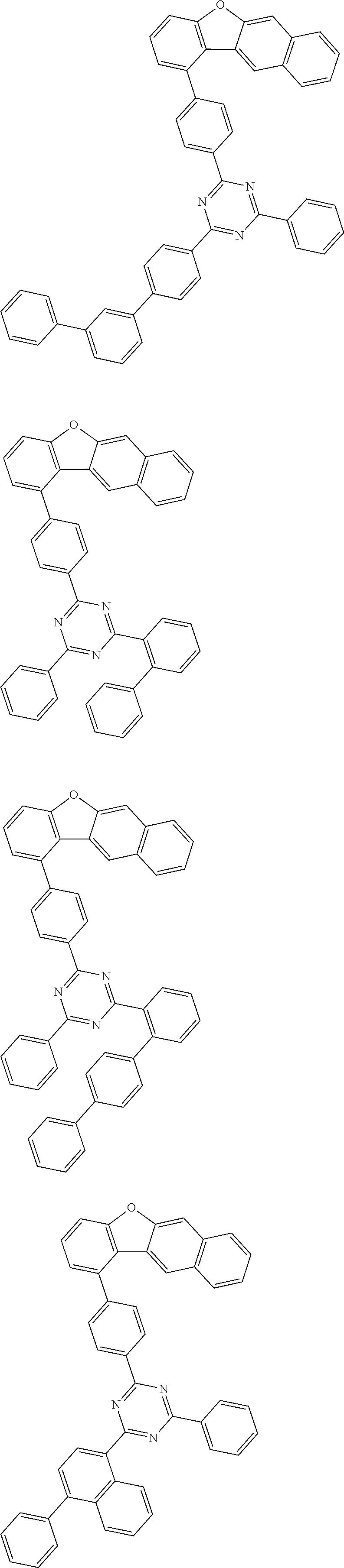 [SMILES: c1ccc(-c2cccc(-c3ccc(-c4nc(-c5ccccc5)nc(-c5ccc(-c6cccc7oc8cc9ccccc9cc8c67)cc5)n4)cc3)c2)cc1 ; c1ccc(-c2nc(-c3ccc(-c4cccc5oc6cc7ccccc7cc6c45)cc3)nc(-c3ccccc3-c3ccccc3)n2)cc1 ; c1ccc(-c2ccc(-c3ccccc3-c3nc(-c4ccccc4)nc(-c4ccc(-c5cccc6oc7cc8ccccc8cc7c56)cc4)n3)cc2)cc1 ; c1ccc(-c2nc(-c3ccc(-c4cccc5oc6cc7ccccc7cc6c45)cc3)nc(-c3ccc(-c4ccccc4)c4ccccc34)n2)cc1]

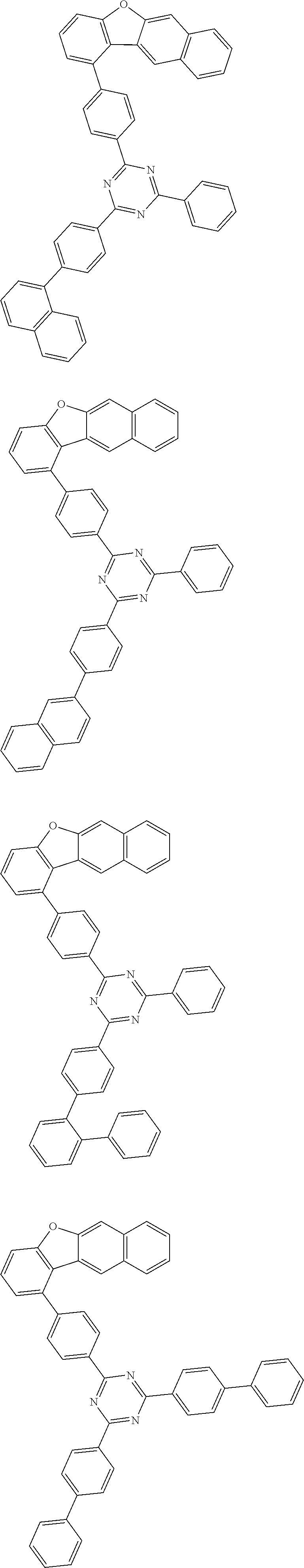 [SMILES: c1ccc(-c2nc(-c3ccc(-c4cccc5ccccc45)cc3)nc(-c3ccc(-c4cccc5oc6cc7ccccc7cc6c45)cc3)n2)cc1 ; c1ccc(-c2nc(-c3ccc(-c4ccc5ccccc5c4)cc3)nc(-c3ccc(-c4cccc5oc6cc7ccccc7cc6c45)cc3)n2)cc1 ; c1ccc(-c2nc(-c3ccc(-c4ccccc4-c4ccccc4)cc3)nc(-c3ccc(-c4cccc5oc6cc7ccccc7cc6c45)cc3)n2)cc1 ; c1ccc(-c2ccc(-c3nc(-c4ccc(-c5ccccc5)cc4)nc(-c4ccc(-c5cccc6oc7cc8ccccc8cc7c56)cc4)n3)cc2)cc1]

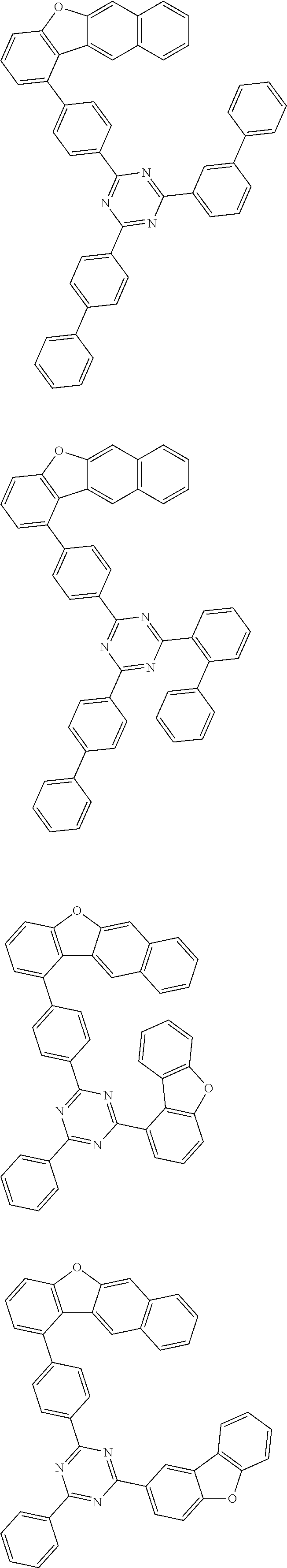 [SMILES: c1ccc(-c2ccc(-c3nc(-c4ccc(-c5cccc6oc7cc8ccccc8cc7c56)cc4)nc(-c4cccc(-c5ccccc5)c4)n3)cc2)cc1 ; c1ccc(-c2ccc(-c3nc(-c4ccc(-c5cccc6oc7cc8ccccc8cc7c56)cc4)nc(-c4ccccc4-c4ccccc4)n3)cc2)cc1 ; c1ccc(-c2nc(-c3ccc(-c4cccc5oc6cc7ccccc7cc6c45)cc3)nc(-c3cccc4oc5ccccc5c34)n2)cc1 ; c1ccc(-c2nc(-c3ccc(-c4cccc5oc6cc7ccccc7cc6c45)cc3)nc(-c3ccc4oc5ccccc5c4c3)n2)cc1]

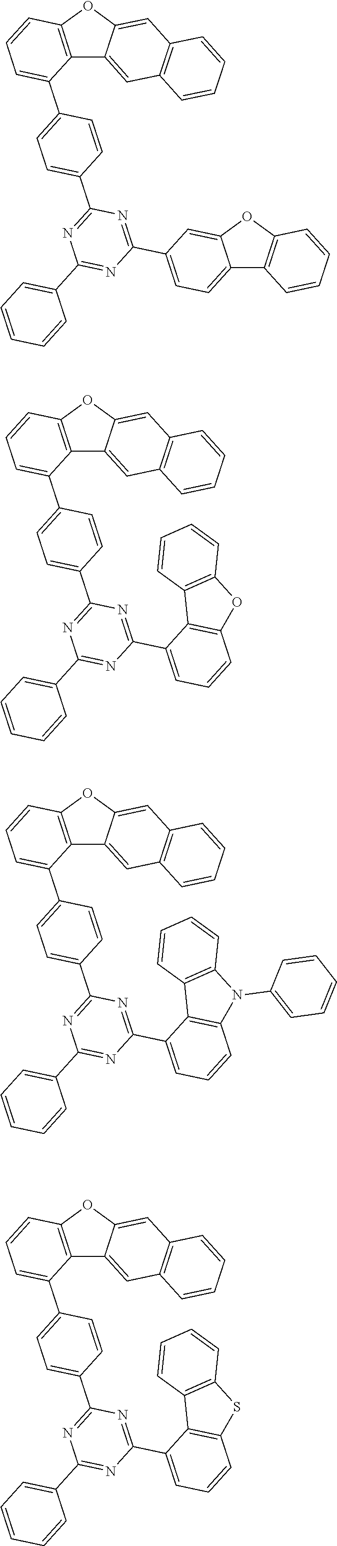 [SMILES: c1ccc(-c2nc(-c3ccc(-c4cccc5oc6cc7ccccc7cc6c45)cc3)nc(-c3ccc4c(c3)oc3ccccc34)n2)cc1 ; c1ccc(-c2nc(-c3ccc(-c4cccc5oc6cc7ccccc7cc6c45)cc3)nc(-c3cccc4oc5ccccc5c34)n2)cc1 ; c1ccc(-c2nc(-c3ccc(-c4cccc5oc6cc7ccccc7cc6c45)cc3)nc(-c3cccc4c3c3ccccc3n4-c3ccccc3)n2)cc1 ; c1ccc(-c2nc(-c3ccc(-c4cccc5oc6cc7ccccc7cc6c45)cc3)nc(-c3cccc4sc5ccccc5c34)n2)cc1]

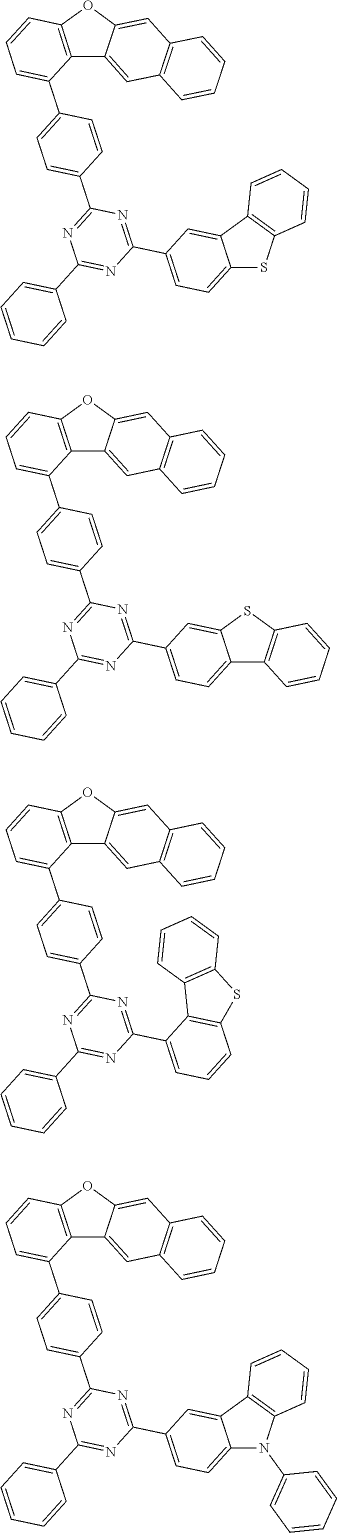 [SMILES: c1ccc(-c2nc(-c3ccc(-c4cccc5oc6cc7ccccc7cc6c45)cc3)nc(-c3ccc4sc5ccccc5c4c3)n2)cc1 ; c1ccc(-c2nc(-c3ccc(-c4cccc5oc6cc7ccccc7cc6c45)cc3)nc(-c3ccc4c(c3)sc3ccccc34)n2)cc1 ; c1ccc(-c2nc(-c3ccc(-c4cccc5oc6cc7ccccc7cc6c45)cc3)nc(-c3cccc4sc5ccccc5c34)n2)cc1 ; c1ccc(-c2nc(-c3ccc(-c4cccc5oc6cc7ccccc7cc6c45)cc3)nc(-c3ccc4c(c3)c3ccccc3n4-c3ccccc3)n2)cc1]

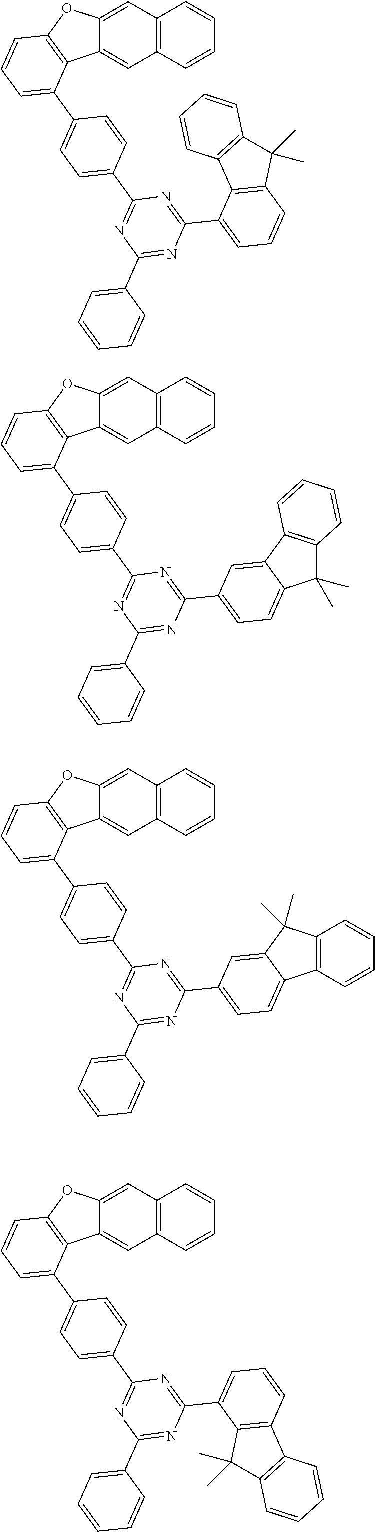

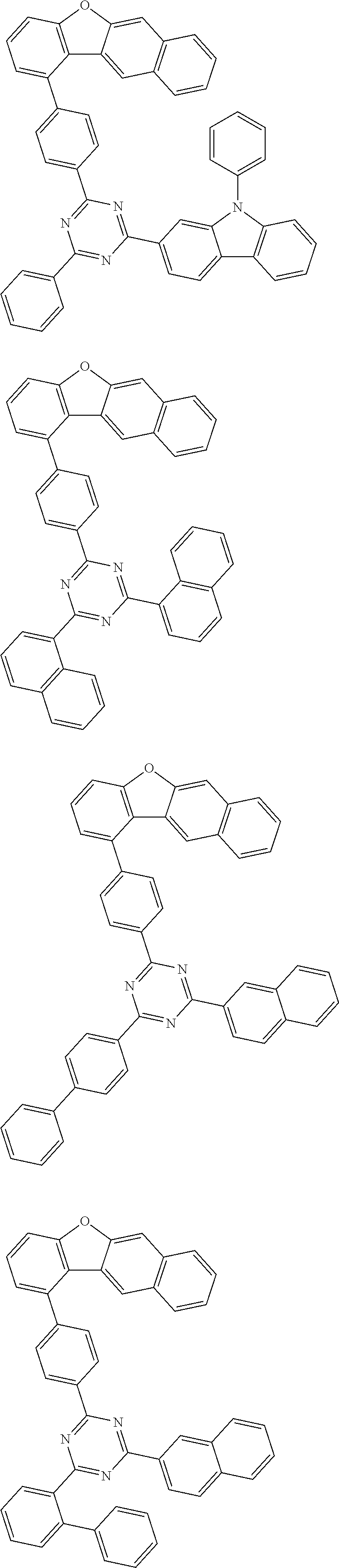 [SMILES: c1ccc(-c2nc(-c3ccc(-c4cccc5oc6cc7ccccc7cc6c45)cc3)nc(-c3ccc4c5ccccc5n(-c5ccccc5)c4c3)n2)cc1 ; c1ccc2cc3c(cc2c1)oc1cccc(-c2ccc(-c4nc(-c5cccc6ccccc56)nc(-c5cccc6ccccc56)n4)cc2)c13 ; c1ccc(-c2ccc(-c3nc(-c4ccc(-c5cccc6oc7cc8ccccc8cc7c56)cc4)nc(-c4ccc5ccccc5c4)n3)cc2)cc1 ; c1ccc(-c2ccccc2-c2nc(-c3ccc(-c4cccc5oc6cc7ccccc7cc6c45)cc3)nc(-c3ccc4ccccc4c3)n2)cc1]

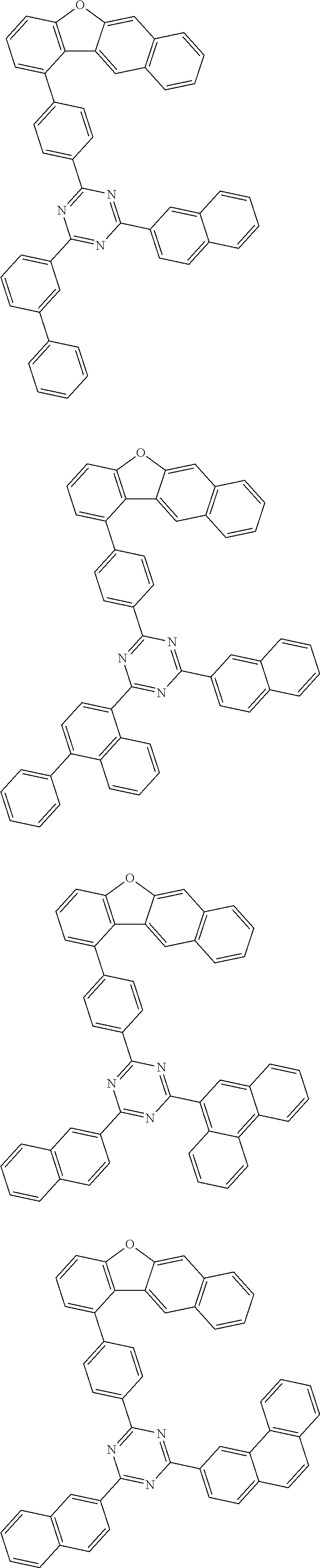 [SMILES: c1ccc(-c2cccc(-c3nc(-c4ccc(-c5cccc6oc7cc8ccccc8cc7c56)cc4)nc(-c4ccc5ccccc5c4)n3)c2)cc1 ; c1ccc(-c2ccc(-c3nc(-c4ccc(-c5cccc6oc7cc8ccccc8cc7c56)cc4)nc(-c4ccc5ccccc5c4)n3)c3ccccc23)cc1 ; c1ccc2cc(-c3nc(-c4ccc(-c5cccc6oc7cc8ccccc8cc7c56)cc4)nc(-c4cc5ccccc5c5ccccc45)n3)ccc2c1 ; c1ccc2cc(-c3nc(-c4ccc(-c5cccc6oc7cc8ccccc8cc7c56)cc4)nc(-c4ccc5ccc6ccccc6c5c4)n3)ccc2c1]

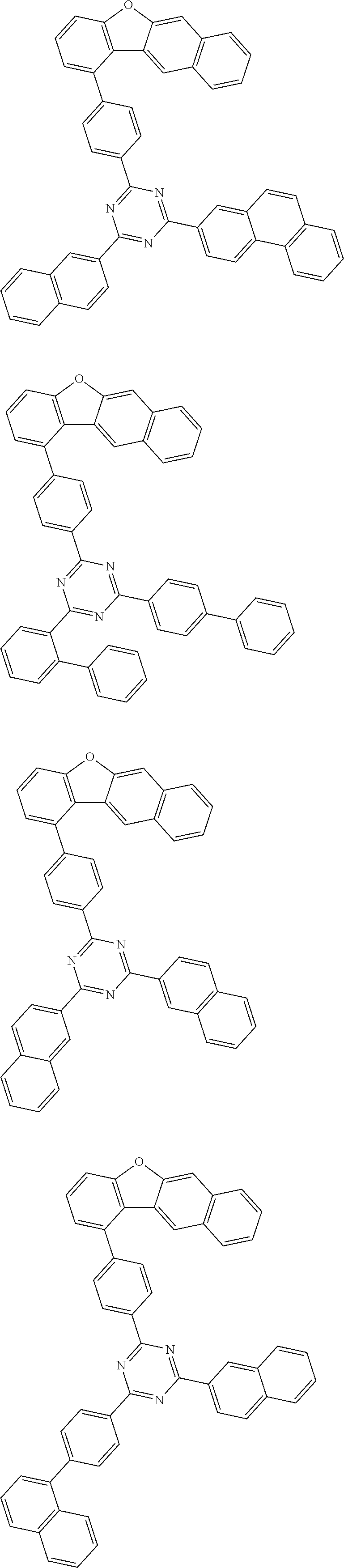 [SMILES: c1ccc2cc(-c3nc(-c4ccc(-c5cccc6oc7cc8ccccc8cc7c56)cc4)nc(-c4ccc5c(ccc6ccccc65)c4)n3)ccc2c1 ; c1ccc(-c2ccc(-c3nc(-c4ccc(-c5cccc6oc7cc8ccccc8cc7c56)cc4)nc(-c4ccccc4-c4ccccc4)n3)cc2)cc1 ; c1ccc2cc(-c3nc(-c4ccc(-c5cccc6oc7cc8ccccc8cc7c56)cc4)nc(-c4ccc5ccccc5c4)n3)ccc2c1 ; c1ccc2cc(-c3nc(-c4ccc(-c5cccc6ccccc56)cc4)nc(-c4ccc(-c5cccc6oc7cc8ccccc8cc7c56)cc4)n3)ccc2c1]

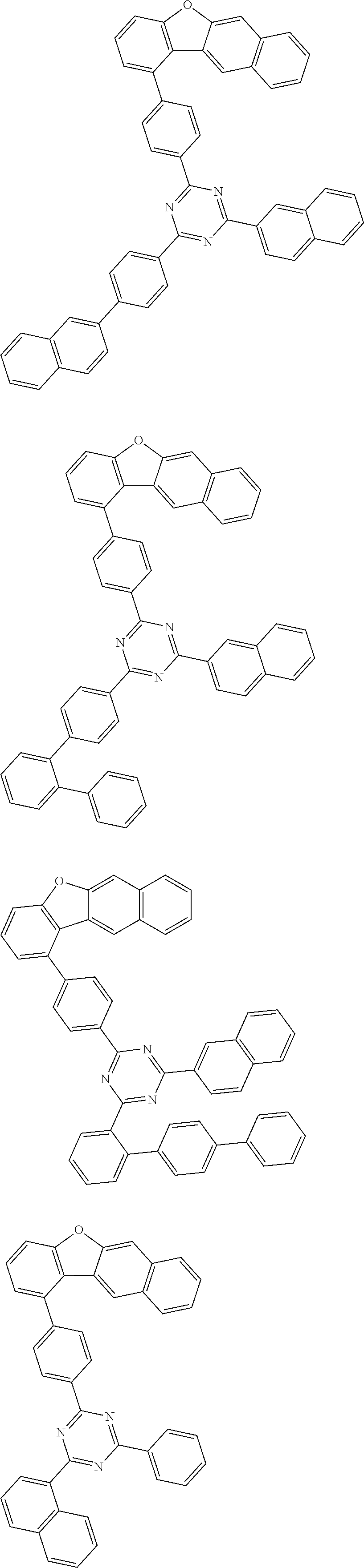 [SMILES: c1ccc2cc(-c3ccc(-c4nc(-c5ccc(-c6cccc7oc8cc9ccccc9cc8c67)cc5)nc(-c5ccc6ccccc6c5)n4)cc3)ccc2c1 ; c1ccc(-c2ccccc2-c2ccc(-c3nc(-c4ccc(-c5cccc6oc7cc8ccccc8cc7c56)cc4)nc(-c4ccc5ccccc5c4)n3)cc2)cc1 ; c1ccc(-c2ccc(-c3ccccc3-c3nc(-c4ccc(-c5cccc6oc7cc8ccccc8cc7c56)cc4)nc(-c4ccc5ccccc5c4)n3)cc2)cc1 ; c1ccc(-c2nc(-c3ccc(-c4cccc5oc6cc7ccccc7cc6c45)cc3)nc(-c3cccc4ccccc34)n2)cc1]

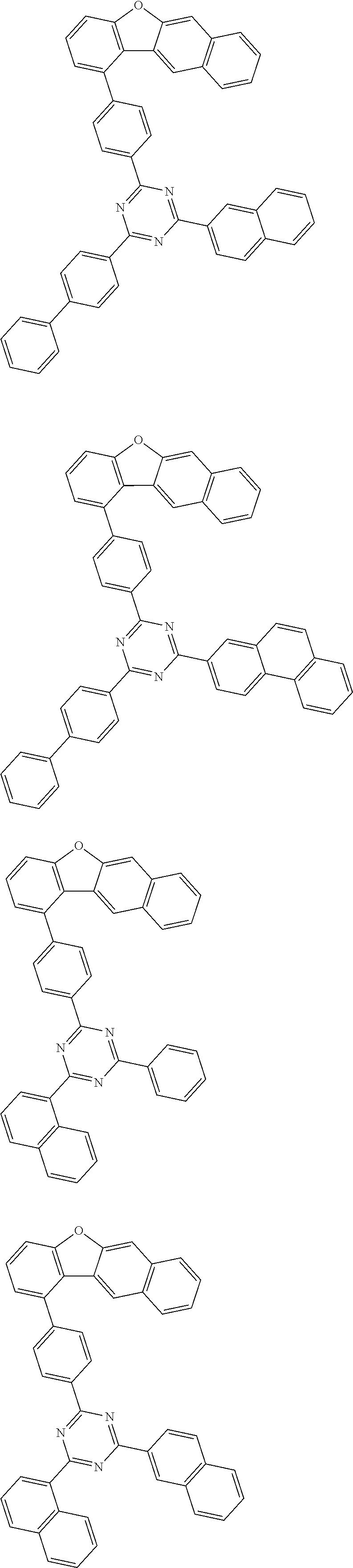 [SMILES: c1ccc(-c2ccc(-c3nc(-c4ccc(-c5cccc6oc7cc8ccccc8cc7c56)cc4)nc(-c4ccc5ccccc5c4)n3)cc2)cc1 ; c1ccc(-c2ccc(-c3nc(-c4ccc(-c5cccc6oc7cc8ccccc8cc7c56)cc4)nc(-c4ccc5c(ccc6ccccc65)c4)n3)cc2)cc1 ; c1ccc(-c2nc(-c3ccc(-c4cccc5oc6cc7ccccc7cc6c45)cc3)nc(-c3cccc4ccccc34)n2)cc1 ; c1ccc2cc(-c3nc(-c4ccc(-c5cccc6oc7cc8ccccc8cc7c56)cc4)nc(-c4cccc5ccccc45)n3)ccc2c1]

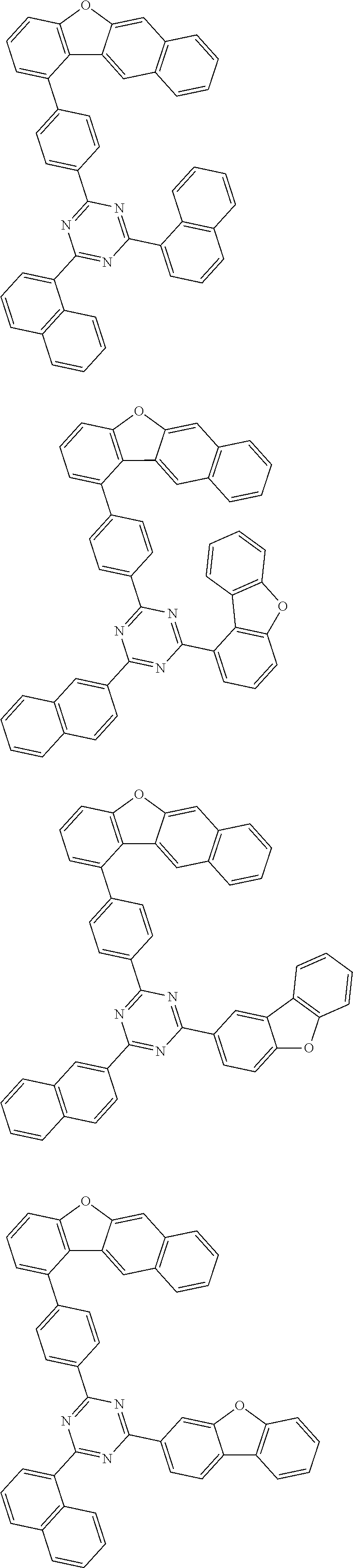 [SMILES: c1ccc2cc3c(cc2c1)oc1cccc(-c2ccc(-c4nc(-c5cccc6ccccc56)nc(-c5cccc6ccccc56)n4)cc2)c13 ; c1ccc2cc(-c3nc(-c4ccc(-c5cccc6oc7cc8ccccc8cc7c56)cc4)nc(-c4cccc5oc6ccccc6c45)n3)ccc2c1 ; c1ccc2cc(-c3nc(-c4ccc(-c5cccc6oc7cc8ccccc8cc7c56)cc4)nc(-c4ccc5oc6ccccc6c5c4)n3)ccc2c1 ; c1ccc2cc3c(cc2c1)oc1cccc(-c2ccc(-c4nc(-c5ccc6c(c5)oc5ccccc56)nc(-c5cccc6ccccc56)n4)cc2)c13]

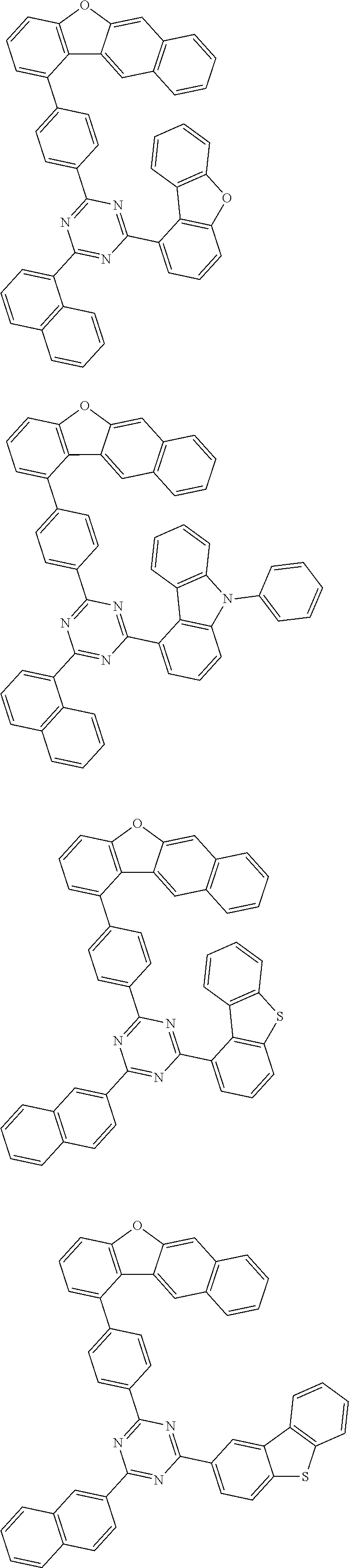

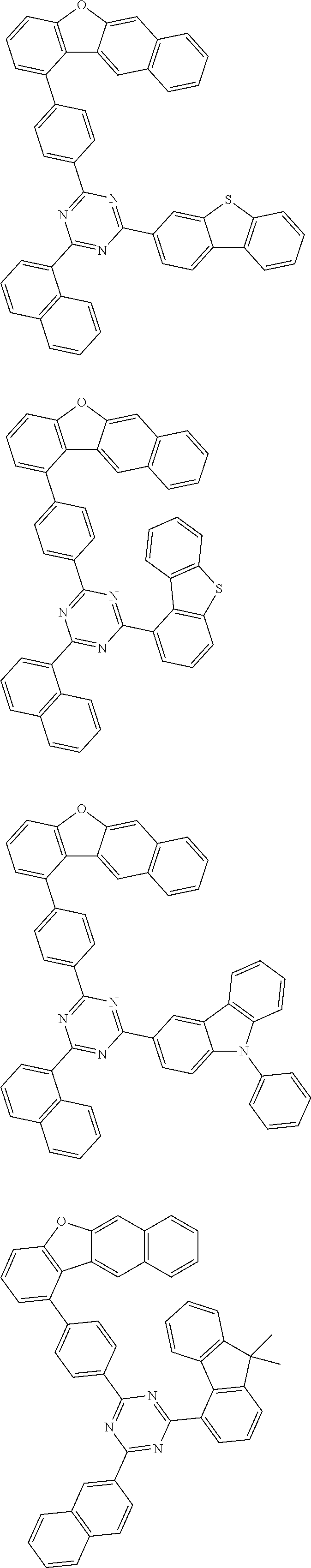 [SMILES: c1ccc2cc3c(cc2c1)oc1cccc(-c2ccc(-c4nc(-c5ccc6c(c5)sc5ccccc56)nc(-c5cccc6ccccc56)n4)cc2)c13 ; c1ccc2cc3c(cc2c1)oc1cccc(-c2ccc(-c4nc(-c5cccc6ccccc56)nc(-c5cccc6sc7ccccc7c56)n4)cc2)c13 ; c1ccc(-n2c3ccccc3c3cc(-c4nc(-c5ccc(-c6cccc7oc8cc9ccccc9cc8c67)cc5)nc(-c5cccc6ccccc56)n4)ccc32)cc1 ; CC1(C)c2ccccc2-c2c(-c3nc(-c4ccc(-c5cccc6oc7cc8ccccc8cc7c56)cc4)nc(-c4ccc5ccccc5c4)n3)cccc21]

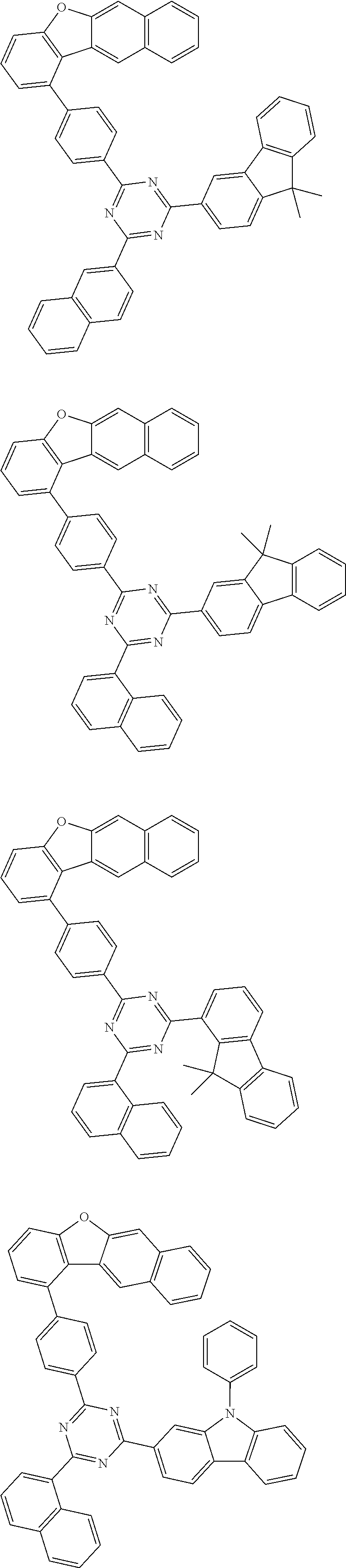 [SMILES: CC1(C)c2ccccc2-c2cc(-c3nc(-c4ccc(-c5cccc6oc7cc8ccccc8cc7c56)cc4)nc(-c4ccc5ccccc5c4)n3)ccc21 ; CC1(C)c2ccccc2-c2ccc(-c3nc(-c4ccc(-c5cccc6oc7cc8ccccc8cc7c56)cc4)nc(-c4cccc5ccccc45)n3)cc21 ; CC1(C)c2ccccc2-c2cccc(-c3nc(-c4ccc(-c5cccc6oc7cc8ccccc8cc7c56)cc4)nc(-c4cccc5ccccc45)n3)c21 ; c1ccc(-n2c3ccccc3c3ccc(-c4nc(-c5ccc(-c6cccc7oc8cc9ccccc9cc8c67)cc5)nc(-c5cccc6ccccc56)n4)cc32)cc1]

Also provided herein is a method for preparing the compound represented by Chemical Formula 1 as shown in the following Reaction Scheme 1.

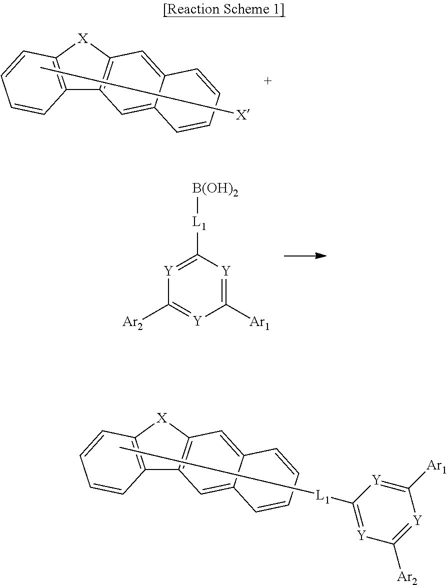

In Reaction Scheme 1, the definition of the remaining substituents except for X′ are the same as defined above, and X is halogen, preferably bromo or chloro. The above reaction is a Suzuki coupling reaction which is preferably carried out in the presence of a palladium catalyst and a base, and a reactive group for the Suzuki coupling reaction can be modified as known in the art. The above preparation method may be further embodied in Preparation Examples described hereinafter.

Preferably, the compound of Chemical Formula 2 is represented by the following Chemical Formula 2-1:

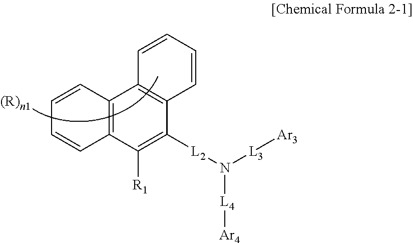

wherein in Chemical Formula 2-1.

R₁ is hydrogen, deuterium, or phenyl,

n1 is an integer of 0 to 8,

L₂, L₃, L₄, Ar₃, Ar₄ and R are the same as defined above.

Preferably, L₂ is phenylene; or phenylene substituted with at least one deuterium. Phenylene substituted with at least one deuterium is preferably any one selected from the group consisting of the following:

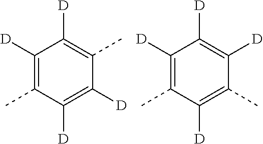

Preferably; L₃ and L₄ are each independently a single bond; phenylene; biphenyldiyl; or naphthylene, each of which except the single bond is independently unsubstituted or substituted with at least one deuterium. When L₃ or L₄ except the single bond is substituted with at least one deuterium; each of them is preferably any one selected from the group consisting of:

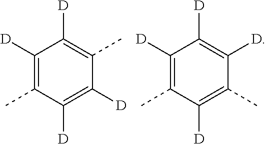

Preferably, Ar₃ and Ar₄ are each independently phenyl, biphenyl, terphenyl, naphthyl; phenanthrenyl, (phenyl)phenanthrenyl, triphenylenyl, phenylnaphthyl, naphthylphenyl, dimethylfluorenyl, diphenylfluorenyl, dibenzofuranyl, (phenyl)dibenzofuranyl, dibenzothiophenyl, (phenyl)dibenzothiophenyl; carbazole-9-yl, or 9-phenyl-9H-carbazolyl, each of which is independently unsubstituted or substituted with at least one deuterium. When Ar₃ or Ar₄ is substituted with at least one deuterium; each of them is preferably any one selected from the group consisting of:

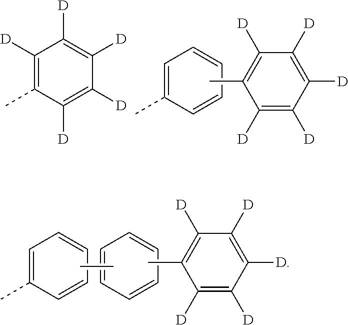

Representative examples of the compound represented by Chemical Formula 2 are as follows:

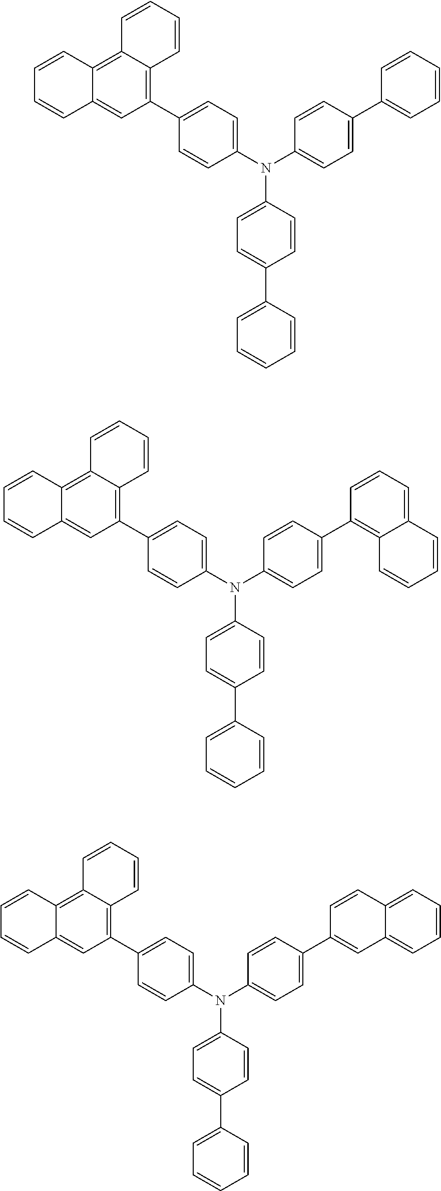 [SMILES: c1ccc(-c2ccc(N(c3ccc(-c4ccccc4)cc3)c3ccc(-c4cc5ccccc5c5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5ccccc5c4)cc3)c3ccc(-c4cc5ccccc5c5ccccc45)cc3)cc2)cc1]

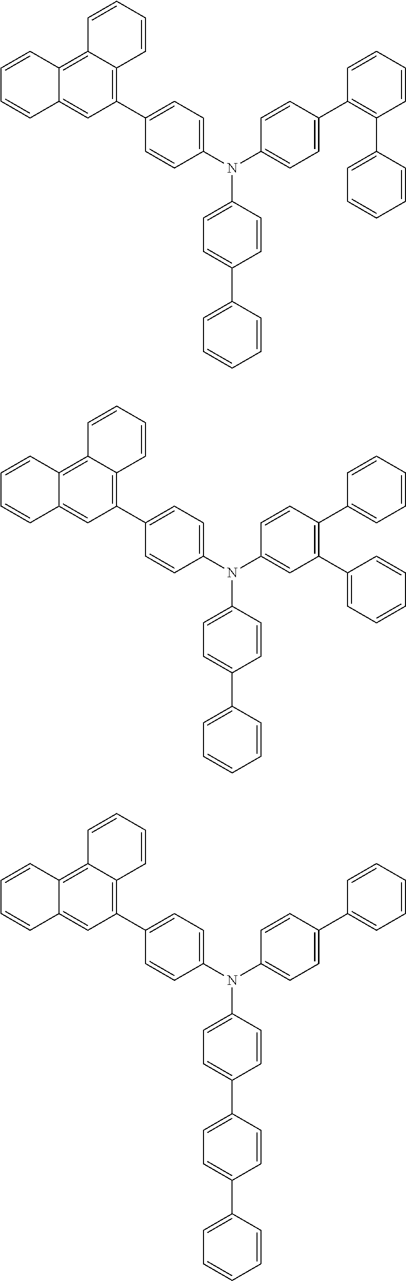 [SMILES: c1ccc(-c2ccc(N(c3ccc(-c4ccccc4-c4ccccc4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c(-c4ccccc4)c3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1]

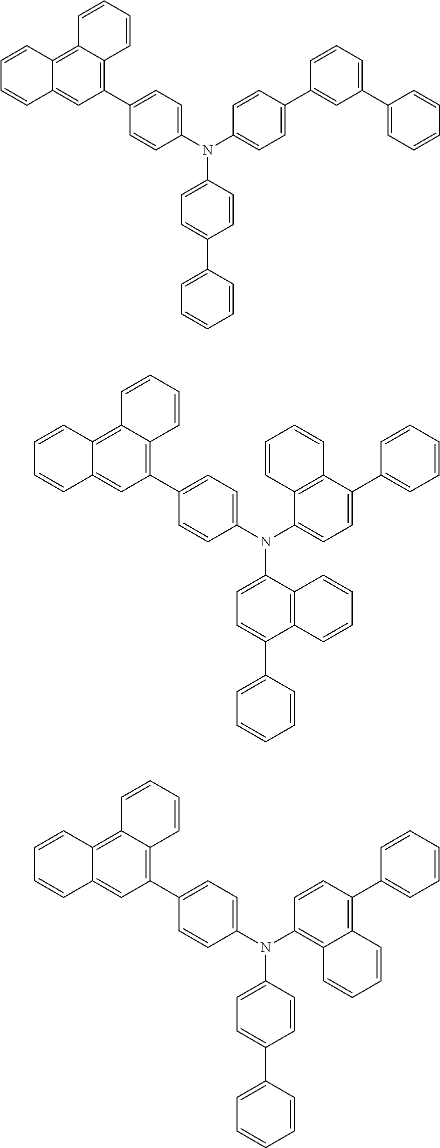 [SMILES: c1ccc(-c2ccc(N(c3ccc(-c4cccc(-c5ccccc5)c4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c4ccccc34)c3ccccc23)cc1 ; c1ccc(-c2ccc(N(c3ccc(-c4cc5ccccc5c5ccccc45)cc3)c3ccc(-c4ccccc4)c4ccccc34)cc2)cc1]

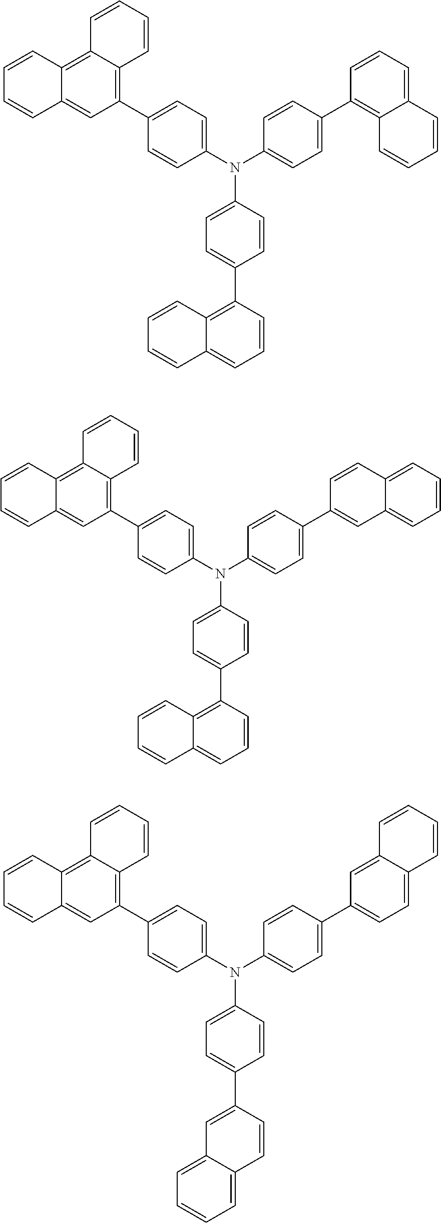 [SMILES: c1ccc2c(-c3ccc(N(c4ccc(-c5cccc6ccccc56)cc4)c4ccc(-c5cc6ccccc6c6ccccc56)cc4)cc3)cccc2c1 ; c1ccc2cc(-c3ccc(N(c4ccc(-c5cccc6ccccc56)cc4)c4ccc(-c5cc6ccccc6c6ccccc56)cc4)cc3)ccc2c1 ; c1ccc2cc(-c3ccc(N(c4ccc(-c5ccc6ccccc6c5)cc4)c4ccc(-c5cc6ccccc6c6ccccc56)cc4)cc3)ccc2c1]

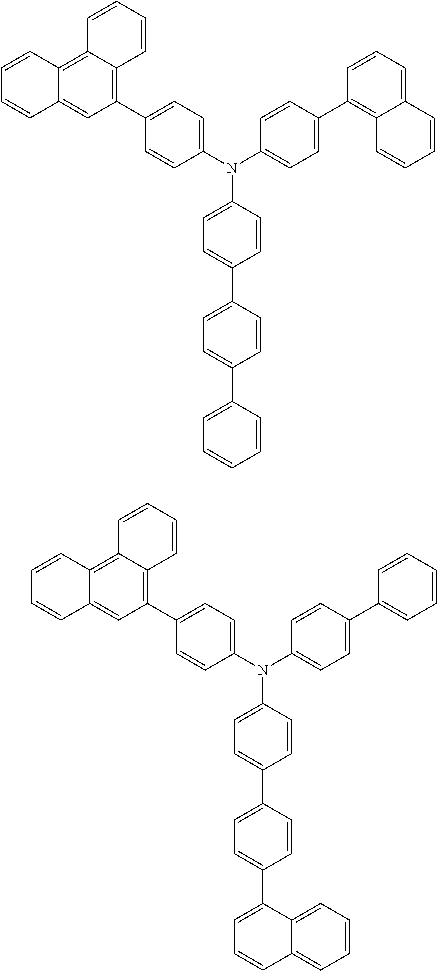 [SMILES: c1ccc(-c2ccc(-c3ccc(N(c4ccc(-c5cccc6ccccc56)cc4)c4ccc(-c5cc6ccccc6c6ccccc56)cc4)cc3)cc2)cc1 ; c1ccc(-c2ccc(N(c3ccc(-c4ccc(-c5cccc6ccccc56)cc4)cc3)c3ccc(-c4cc5ccccc5c5ccccc45)cc3)cc2)cc1]

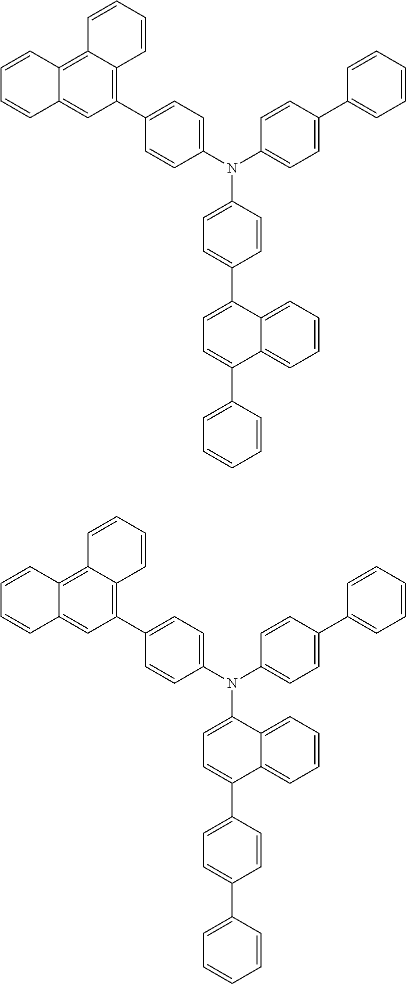 [SMILES: c1ccc(-c2ccc(N(c3ccc(-c4ccc(-c5ccccc5)c5ccccc45)cc3)c3ccc(-c4cc5ccccc5c5ccccc45)cc3)cc2)cc1 ; c1ccc(-c2ccc(-c3ccc(N(c4ccc(-c5ccccc5)cc4)c4ccc(-c5cc6ccccc6c6ccccc56)cc4)c4ccccc34)cc2)cc1]

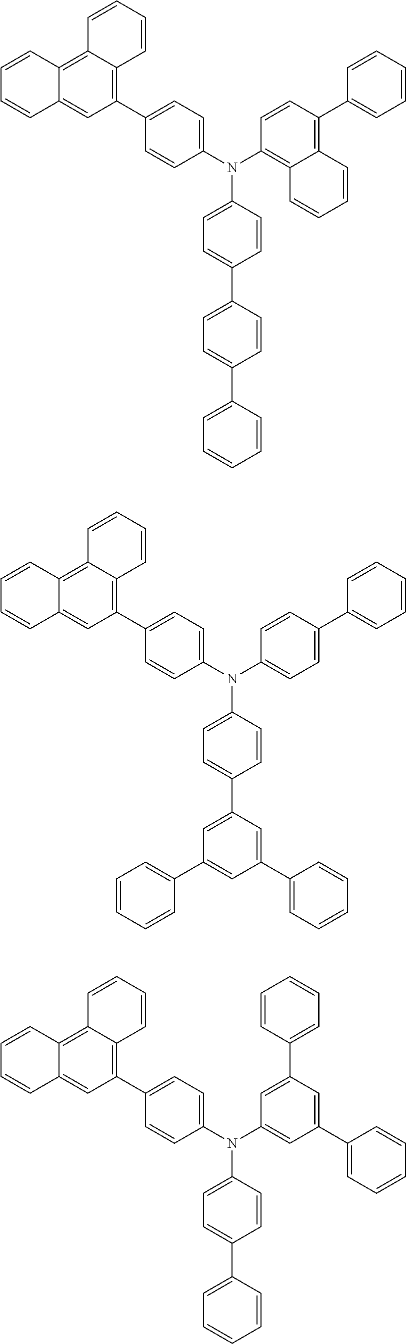 [SMILES: c1ccc(-c2ccc(-c3ccc(N(c4ccc(-c5cc6ccccc6c6ccccc56)cc4)c4ccc(-c5ccccc5)c5ccccc45)cc3)cc2)cc1 ; c1ccc(-c2ccc(N(c3ccc(-c4cc(-c5ccccc5)cc(-c5ccccc5)c4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3cc(-c4ccccc4)cc(-c4ccccc4)c3)cc2)cc1]

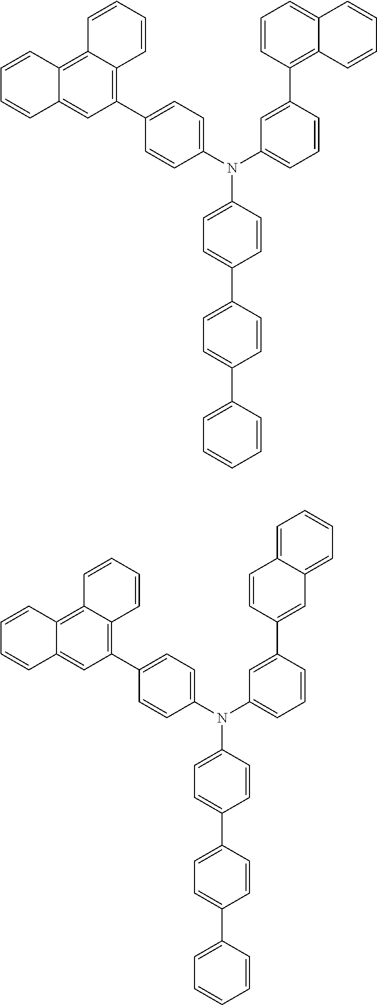 [SMILES: c1ccc(-c2ccc(-c3ccc(N(c4ccc(-c5cc6ccccc6c6ccccc56)cc4)c4cccc(-c5cccc6ccccc56)c4)cc3)cc2)cc1 ; c1ccc(-c2ccc(-c3ccc(N(c4ccc(-c5cc6ccccc6c6ccccc56)cc4)c4cccc(-c5ccc6ccccc6c5)c4)cc3)cc2)cc1]

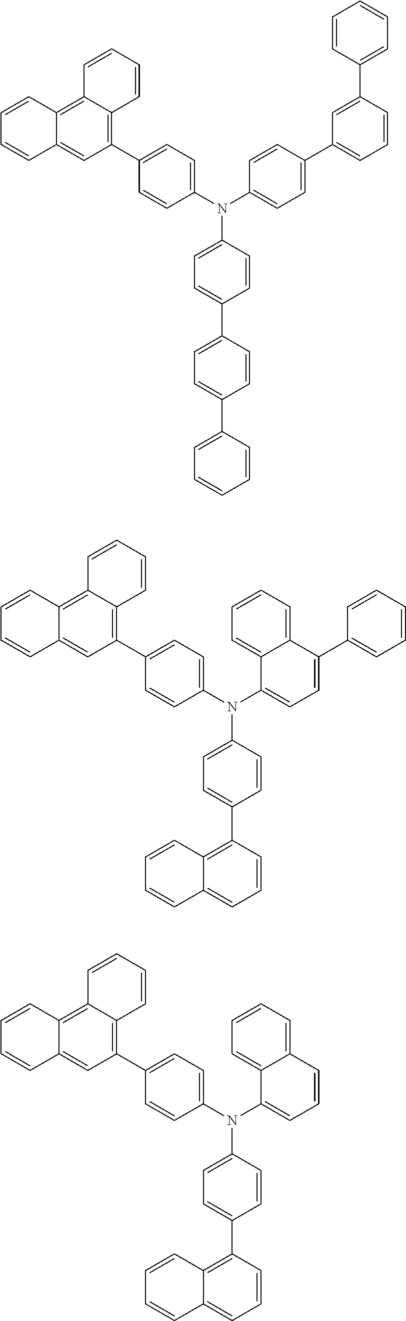 [SMILES: c1ccc(-c2ccc(-c3ccc(N(c4ccc(-c5cccc(-c6ccccc6)c5)cc4)c4ccc(-c5cc6ccccc6c6ccccc56)cc4)cc3)cc2)cc1 ; c1ccc(-c2ccc(N(c3ccc(-c4cccc5ccccc45)cc3)c3ccc(-c4cc5ccccc5c5ccccc45)cc3)c3ccccc23)cc1 ; c1ccc2c(-c3ccc(N(c4ccc(-c5cc6ccccc6c6ccccc56)cc4)c4cccc5ccccc45)cc3)cccc2c1]

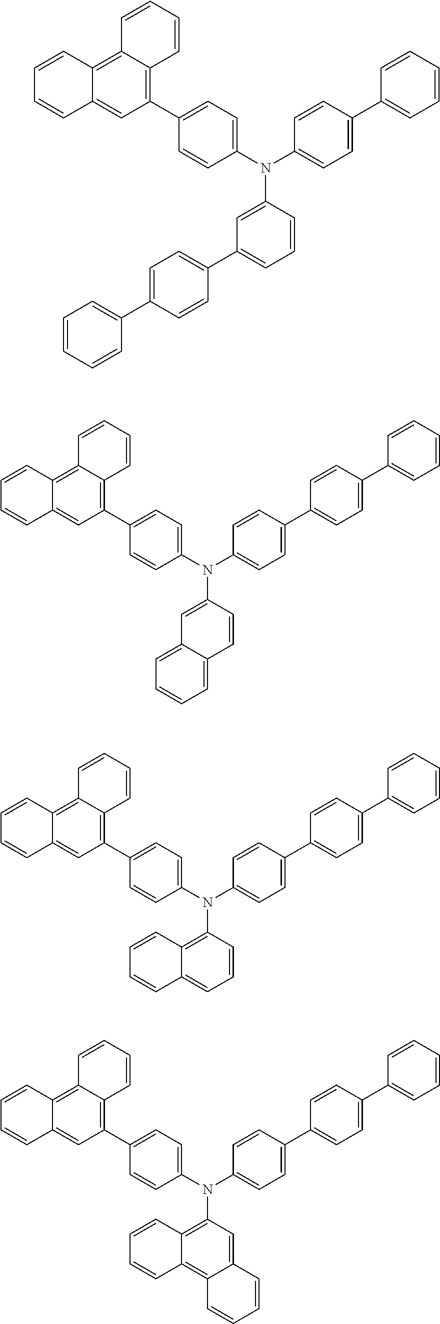 [SMILES: c1ccc(-c2ccc(-c3cccc(N(c4ccc(-c5ccccc5)cc4)c4ccc(-c5cc6ccccc6c6ccccc56)cc4)c3)cc2)cc1 ; c1ccc(-c2ccc(-c3ccc(N(c4ccc(-c5cc6ccccc6c6ccccc56)cc4)c4ccc5ccccc5c4)cc3)cc2)cc1 ; c1ccc(-c2ccc(-c3ccc(N(c4ccc(-c5cc6ccccc6c6ccccc56)cc4)c4cccc5ccccc45)cc3)cc2)cc1 ; c1ccc(-c2ccc(-c3ccc(N(c4ccc(-c5cc6ccccc6c6ccccc56)cc4)c4cc5ccccc5c5ccccc45)cc3)cc2)cc1]

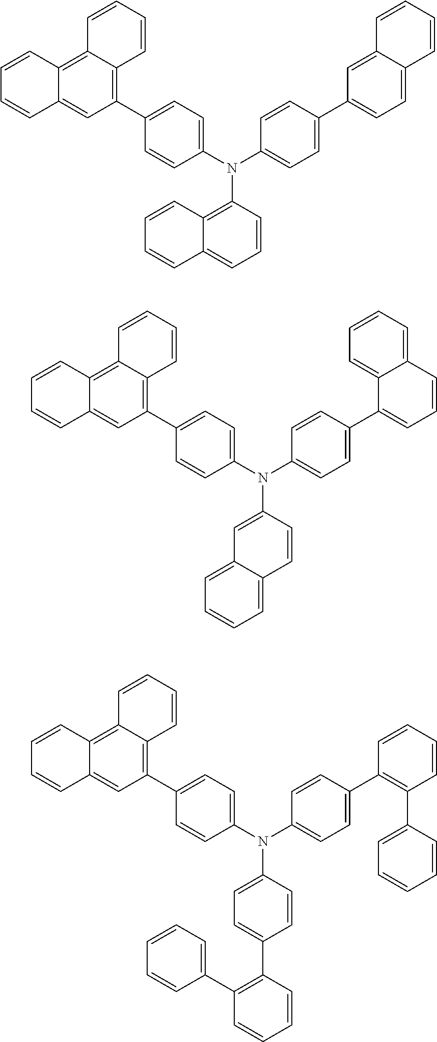 [SMILES: c1ccc2cc(-c3ccc(N(c4ccc(-c5cc6ccccc6c6ccccc56)cc4)c4cccc5ccccc45)cc3)ccc2c1 ; c1ccc2cc(N(c3ccc(-c4cccc5ccccc45)cc3)c3ccc(-c4cc5ccccc5c5ccccc45)cc3)ccc2c1 ; c1ccc(-c2ccccc2-c2ccc(N(c3ccc(-c4ccccc4-c4ccccc4)cc3)c3ccc(-c4cc5ccccc5c5ccccc45)cc3)cc2)cc1]

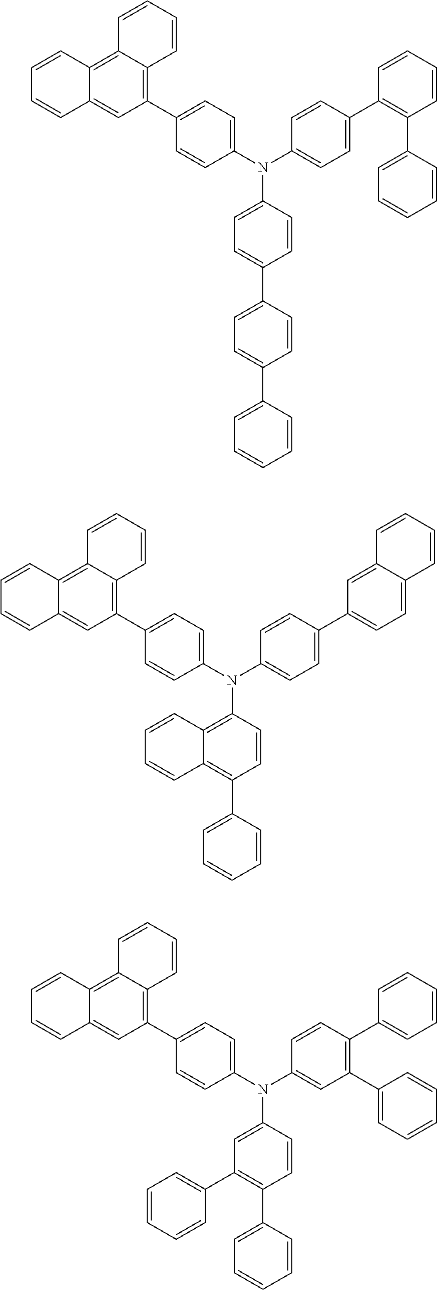 [SMILES: c1ccc(-c2ccc(-c3ccc(N(c4ccc(-c5ccccc5-c5ccccc5)cc4)c4ccc(-c5cc6ccccc6c6ccccc56)cc4)cc3)cc2)cc1 ; c1ccc(-c2ccc(N(c3ccc(-c4ccc5ccccc5c4)cc3)c3ccc(-c4cc5ccccc5c5ccccc45)cc3)c3ccccc23)cc1 ; c1ccc(-c2ccc(N(c3ccc(-c4cc5ccccc5c5ccccc45)cc3)c3ccc(-c4ccccc4)c(-c4ccccc4)c3)cc2-c2ccccc2)cc1]

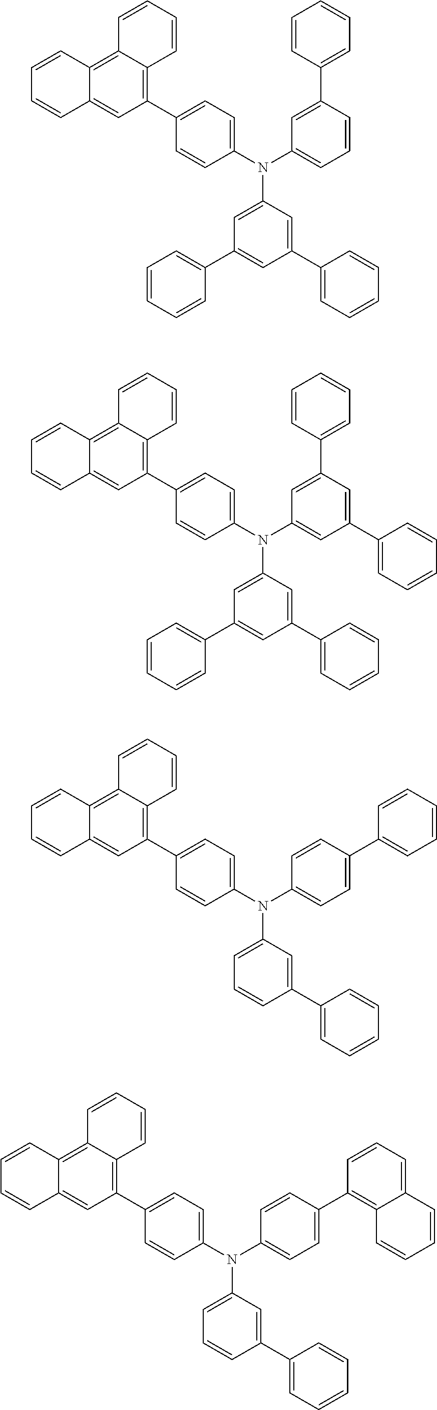 [SMILES: c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2cc(-c3ccccc3)cc(N(c3ccc(-c4cc5ccccc5c5ccccc45)cc3)c3cc(-c4ccccc4)cc(-c4ccccc4)c3)c2)cc1 ; c1ccc(-c2ccc(N(c3ccc(-c4cc5ccccc5c5ccccc45)cc3)c3cccc(-c4ccccc4)c3)cc2)cc1 ; c1ccc(-c2cccc(N(c3ccc(-c4cccc5ccccc45)cc3)c3ccc(-c4cc5ccccc5c5ccccc45)cc3)c2)cc1]

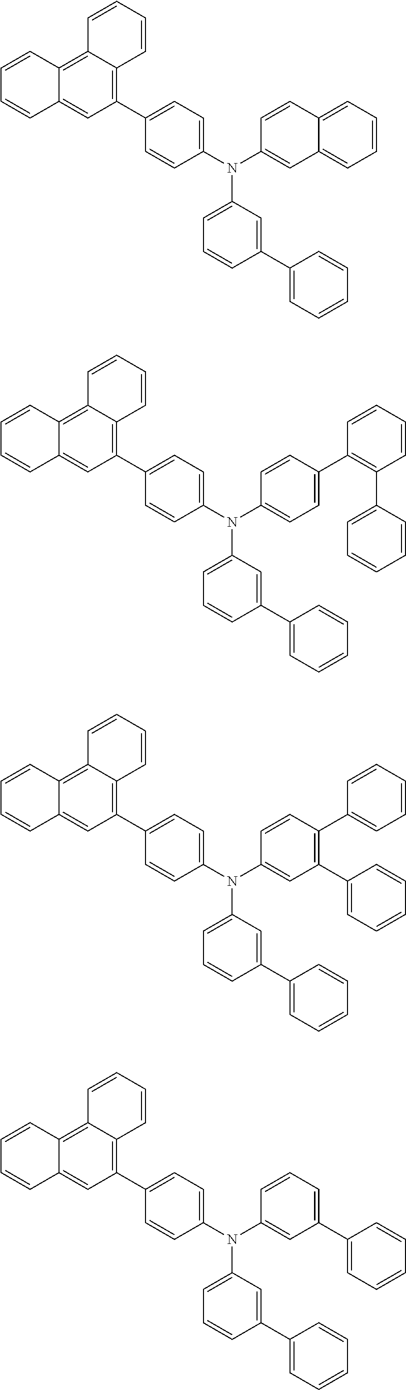 [SMILES: c1ccc(-c2cccc(N(c3ccc(-c4cc5ccccc5c5ccccc45)cc3)c3ccc4ccccc4c3)c2)cc1 ; c1ccc(-c2cccc(N(c3ccc(-c4ccccc4-c4ccccc4)cc3)c3ccc(-c4cc5ccccc5c5ccccc45)cc3)c2)cc1 ; c1ccc(-c2cccc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cccc(-c4ccccc4)c3)c2)cc1]

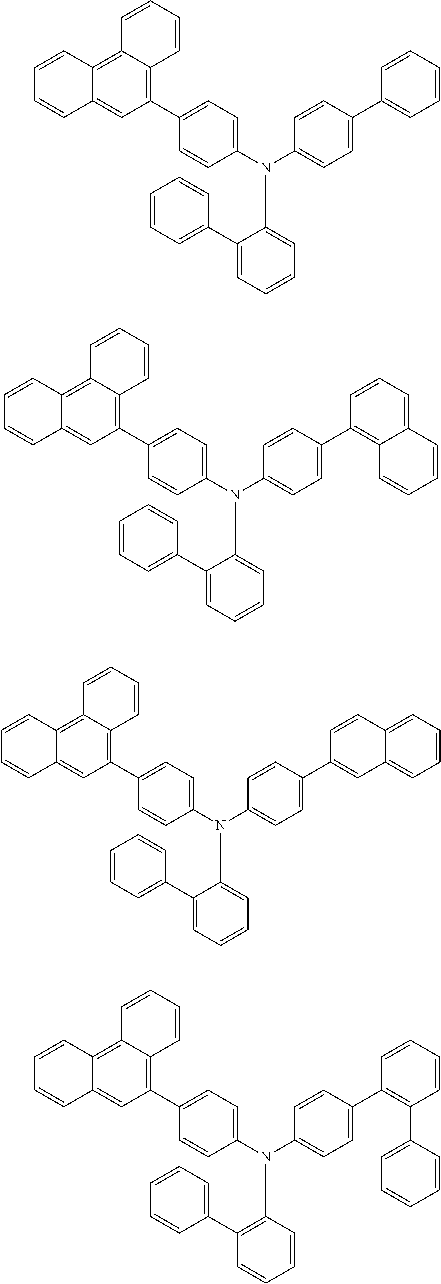 [SMILES: c1ccc(-c2ccc(N(c3ccc(-c4cc5ccccc5c5ccccc45)cc3)c3ccccc3-c3ccccc3)cc2)cc1 ; c1ccc(-c2ccccc2N(c2ccc(-c3cccc4ccccc34)cc2)c2ccc(-c3cc4ccccc4c4ccccc34)cc2)cc1 ; c1ccc(-c2ccccc2N(c2ccc(-c3ccc4ccccc4c3)cc2)c2ccc(-c3cc4ccccc4c4ccccc34)cc2)cc1 ; c1ccc(-c2ccccc2-c2ccc(N(c3ccc(-c4cc5ccccc5c5ccccc45)cc3)c3ccccc3-c3ccccc3)cc2)cc1]

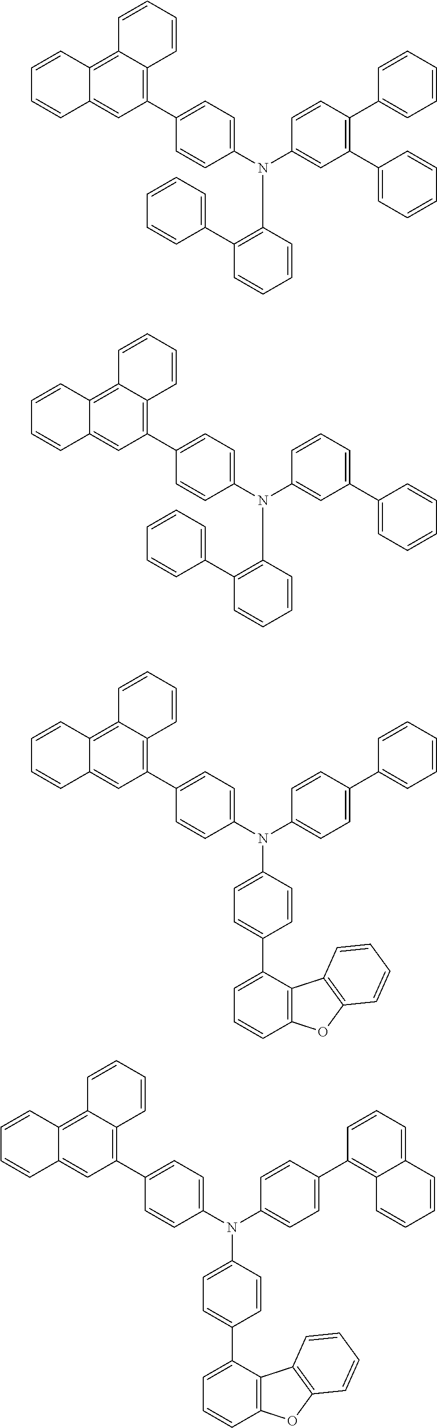 [SMILES: c1ccc(-c2ccc(N(c3ccc(-c4cc5ccccc5c5ccccc45)cc3)c3ccccc3-c3ccccc3)cc2-c2ccccc2)cc1 ; c1ccc(-c2cccc(N(c3ccc(-c4cc5ccccc5c5ccccc45)cc3)c3ccccc3-c3ccccc3)c2)cc1 ; c1ccc(-c2ccc(N(c3ccc(-c4cc5ccccc5c5ccccc45)cc3)c3ccc(-c4cccc5oc6ccccc6c45)cc3)cc2)cc1 ; c1ccc2c(-c3ccc(N(c4ccc(-c5cc6ccccc6c6ccccc56)cc4)c4ccc(-c5cccc6oc7ccccc7c56)cc4)cc3)cccc2c1]

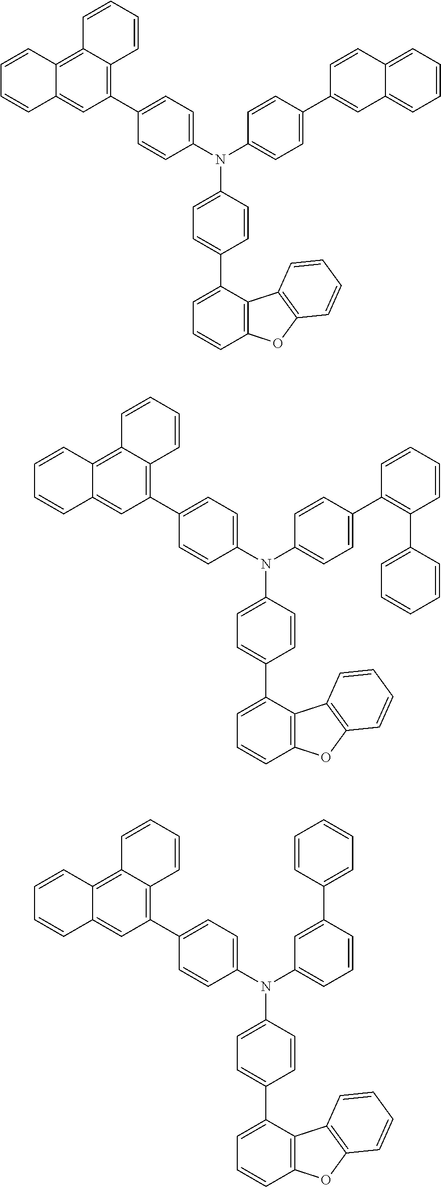 [SMILES: c1ccc2cc(-c3ccc(N(c4ccc(-c5cc6ccccc6c6ccccc56)cc4)c4ccc(-c5cccc6oc7ccccc7c56)cc4)cc3)ccc2c1 ; c1ccc(-c2ccccc2-c2ccc(N(c3ccc(-c4cc5ccccc5c5ccccc45)cc3)c3ccc(-c4cccc5oc6ccccc6c45)cc3)cc2)cc1 ; c1ccc(-c2cccc(N(c3ccc(-c4cc5ccccc5c5ccccc45)cc3)c3ccc(-c4cccc5oc6ccccc6c45)cc3)c2)cc1]

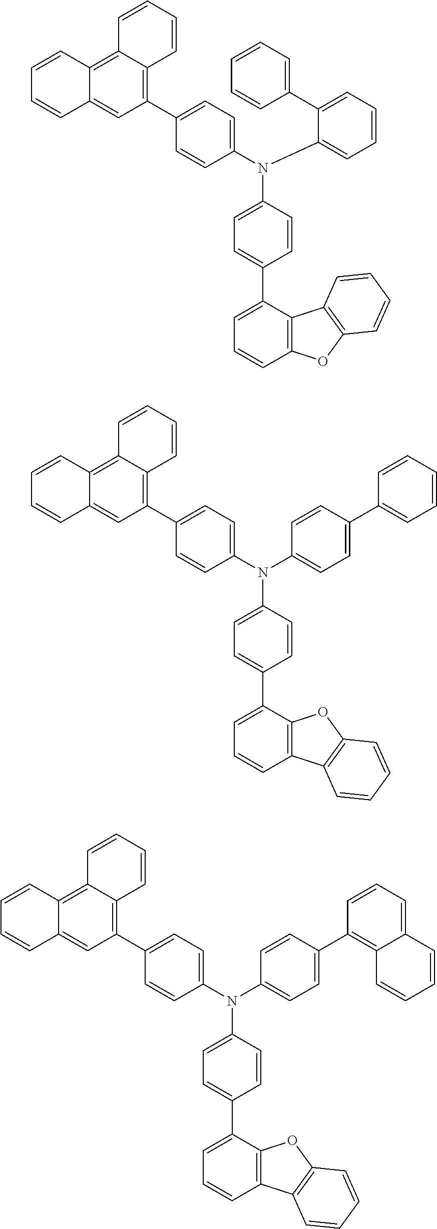 [SMILES: c1ccc(-c2ccccc2N(c2ccc(-c3cc4ccccc4c4ccccc34)cc2)c2ccc(-c3cccc4oc5ccccc5c34)cc2)cc1 ; c1ccc(-c2ccc(N(c3ccc(-c4cc5ccccc5c5ccccc45)cc3)c3ccc(-c4cccc5c4oc4ccccc45)cc3)cc2)cc1 ; c1ccc2c(-c3ccc(N(c4ccc(-c5cc6ccccc6c6ccccc56)cc4)c4ccc(-c5cccc6c5oc5ccccc56)cc4)cc3)cccc2c1]

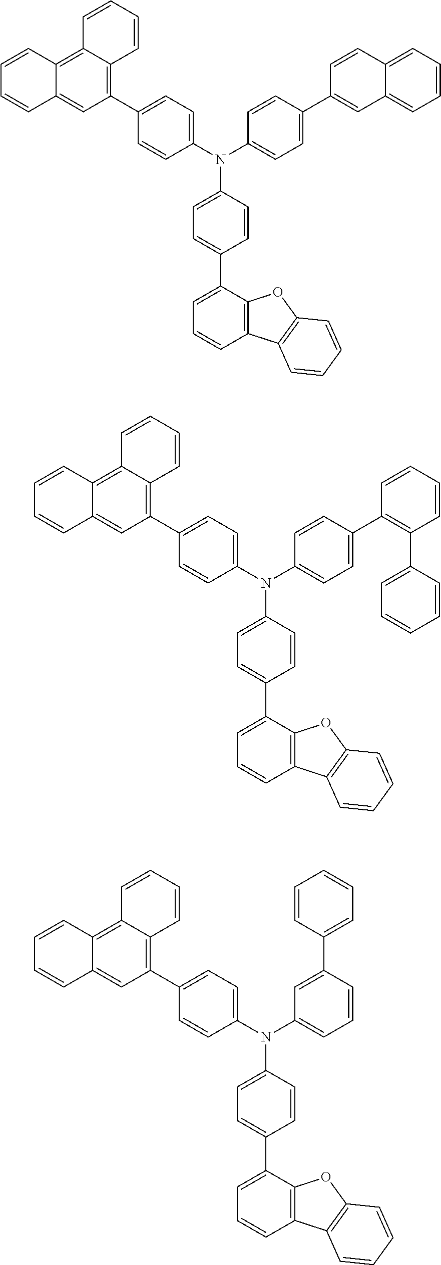 [SMILES: c1ccc2cc(-c3ccc(N(c4ccc(-c5cc6ccccc6c6ccccc56)cc4)c4ccc(-c5cccc6c5oc5ccccc56)cc4)cc3)ccc2c1 ; c1ccc(-c2ccccc2-c2ccc(N(c3ccc(-c4cc5ccccc5c5ccccc45)cc3)c3ccc(-c4cccc5c4oc4ccccc45)cc3)cc2)cc1 ; c1ccc(-c2cccc(N(c3ccc(-c4cc5ccccc5c5ccccc45)cc3)c3ccc(-c4cccc5c4oc4ccccc45)cc3)c2)cc1]

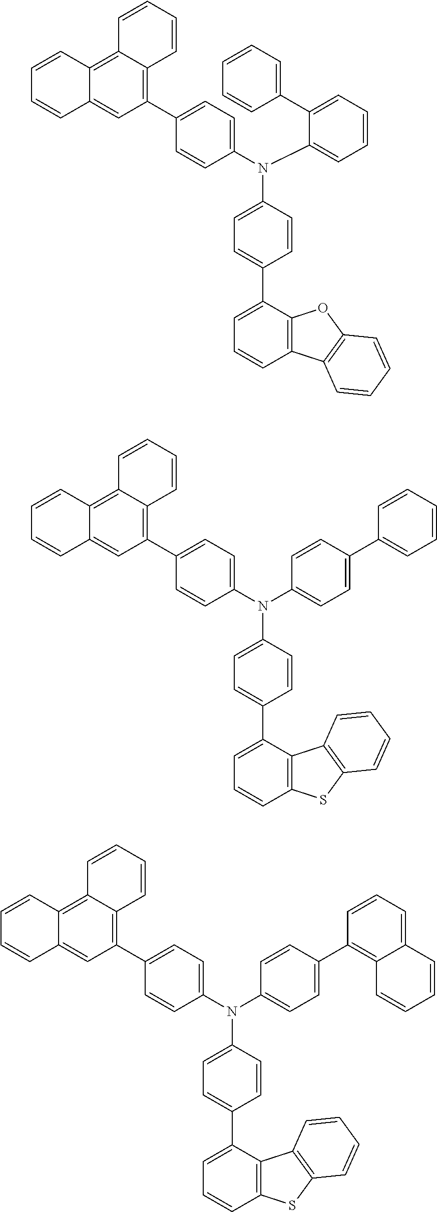 [SMILES: c1ccc(-c2ccccc2N(c2ccc(-c3cc4ccccc4c4ccccc34)cc2)c2ccc(-c3cccc4c3oc3ccccc34)cc2)cc1 ; c1ccc(-c2ccc(N(c3ccc(-c4cc5ccccc5c5ccccc45)cc3)c3ccc(-c4cccc5sc6ccccc6c45)cc3)cc2)cc1 ; c1ccc2c(-c3ccc(N(c4ccc(-c5cc6ccccc6c6ccccc56)cc4)c4ccc(-c5cccc6sc7ccccc7c56)cc4)cc3)cccc2c1]

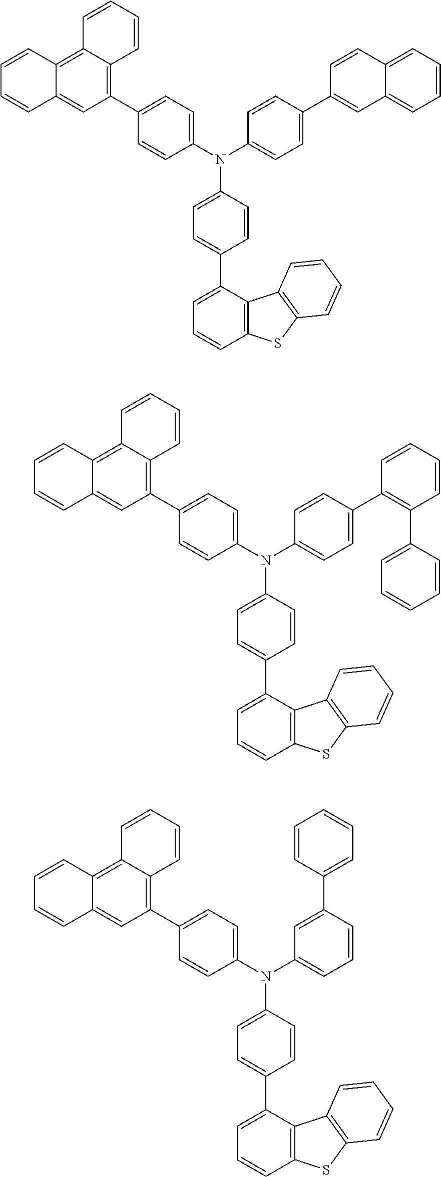 [SMILES: c1ccc2cc(-c3ccc(N(c4ccc(-c5cc6ccccc6c6ccccc56)cc4)c4ccc(-c5cccc6sc7ccccc7c56)cc4)cc3)ccc2c1 ; c1ccc(-c2ccccc2-c2ccc(N(c3ccc(-c4cc5ccccc5c5ccccc45)cc3)c3ccc(-c4cccc5sc6ccccc6c45)cc3)cc2)cc1 ; c1ccc(-c2cccc(N(c3ccc(-c4cc5ccccc5c5ccccc45)cc3)c3ccc(-c4cccc5sc6ccccc6c45)cc3)c2)cc1]

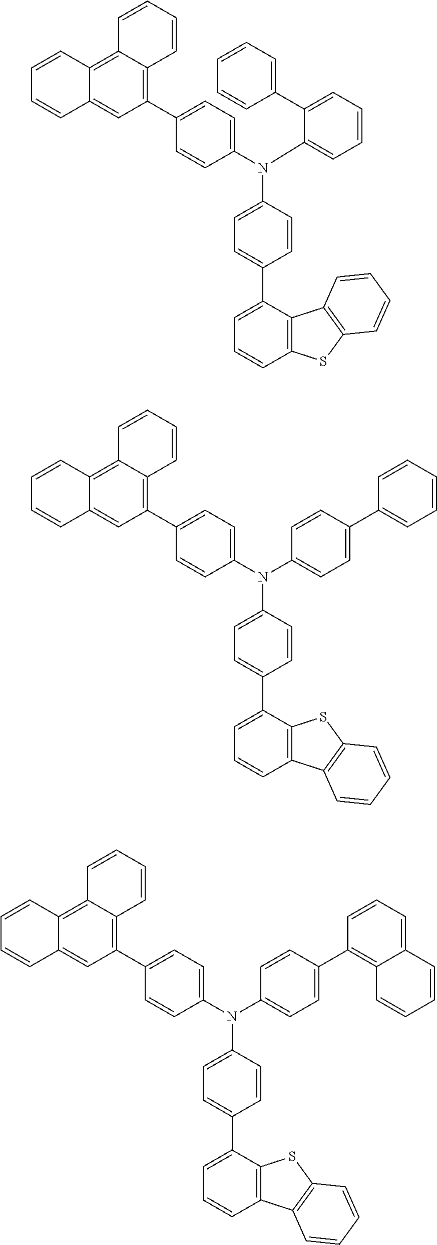 [SMILES: c1ccc(-c2ccccc2N(c2ccc(-c3cc4ccccc4c4ccccc34)cc2)c2ccc(-c3cccc4sc5ccccc5c34)cc2)cc1 ; c1ccc(-c2ccc(N(c3ccc(-c4cc5ccccc5c5ccccc45)cc3)c3ccc(-c4cccc5c4sc4ccccc45)cc3)cc2)cc1 ; c1ccc2c(-c3ccc(N(c4ccc(-c5cc6ccccc6c6ccccc56)cc4)c4ccc(-c5cccc6c5sc5ccccc56)cc4)cc3)cccc2c1]

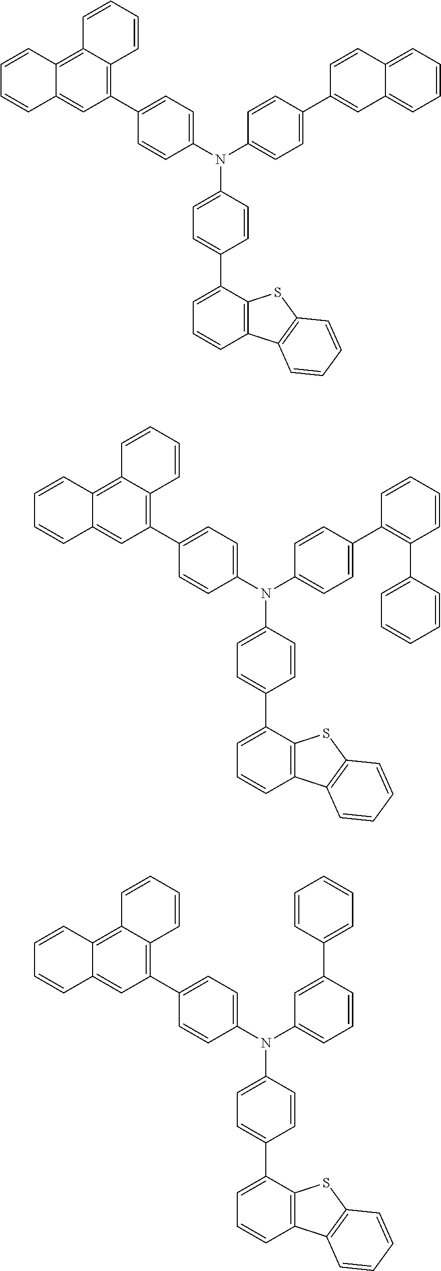 [SMILES: c1ccc2cc(-c3ccc(N(c4ccc(-c5cc6ccccc6c6ccccc56)cc4)c4ccc(-c5cccc6c5sc5ccccc56)cc4)cc3)ccc2c1 ; c1ccc(-c2ccccc2-c2ccc(N(c3ccc(-c4cc5ccccc5c5ccccc45)cc3)c3ccc(-c4cccc5c4sc4ccccc45)cc3)cc2)cc1 ; c1ccc(-c2cccc(N(c3ccc(-c4cc5ccccc5c5ccccc45)cc3)c3ccc(-c4cccc5c4sc4ccccc45)cc3)c2)cc1]

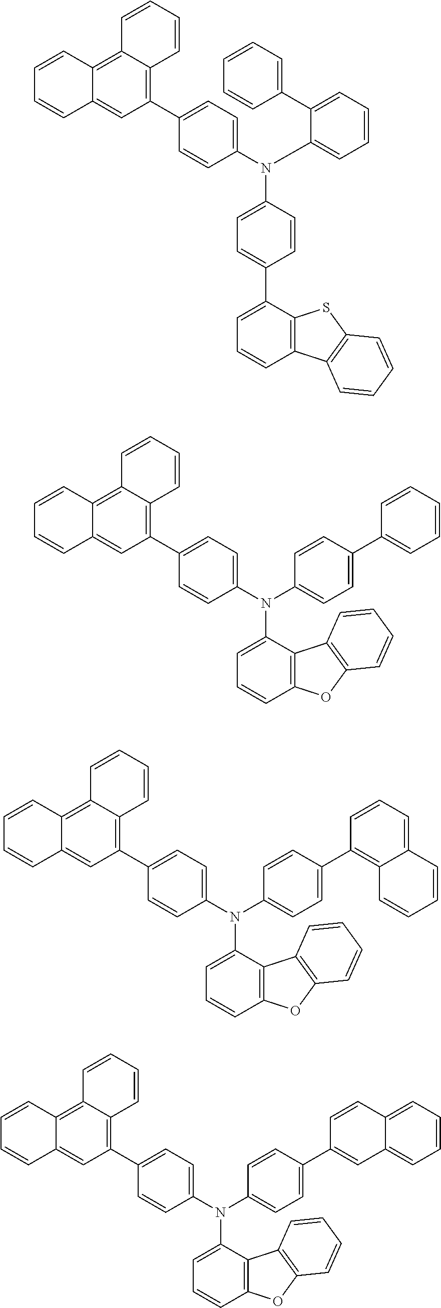 [SMILES: c1ccc(-c2ccccc2N(c2ccc(-c3cc4ccccc4c4ccccc34)cc2)c2ccc(-c3cccc4c3sc3ccccc34)cc2)cc1 ; c1ccc(-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)cc3)cccc2c1 ; c1ccc2cc(-c3ccc(N(c4ccc(-c5cc6ccccc6c6ccccc56)cc4)c4cccc5oc6ccccc6c45)cc3)ccc2c1]

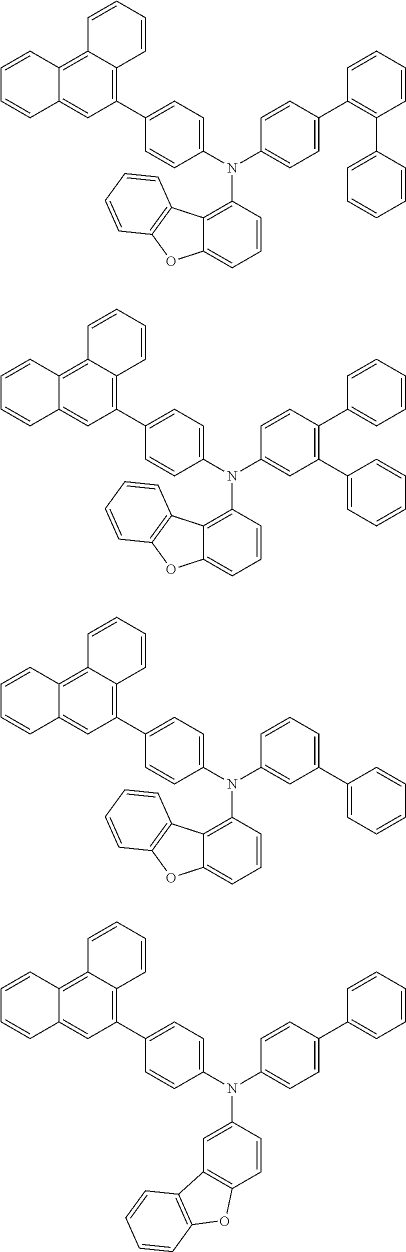 [SMILES: c1ccc(-c2ccccc2-c2ccc(N(c3ccc(-c4cc5ccccc5c5ccccc45)cc3)c3cccc4oc5ccccc5c34)cc2)cc1 ; c1ccc(-c2ccc(N(c3ccc(-c4cc5ccccc5c5ccccc45)cc3)c3cccc4oc5ccccc5c34)cc2-c2ccccc2)cc1 ; c1ccc(-c2cccc(N(c3ccc(-c4cc5ccccc5c5ccccc45)cc3)c3cccc4oc5ccccc5c34)c2)cc1 ; c1ccc(-c2ccc(N(c3ccc(-c4cc5ccccc5c5ccccc45)cc3)c3ccc4oc5ccccc5c4c3)cc2)cc1]

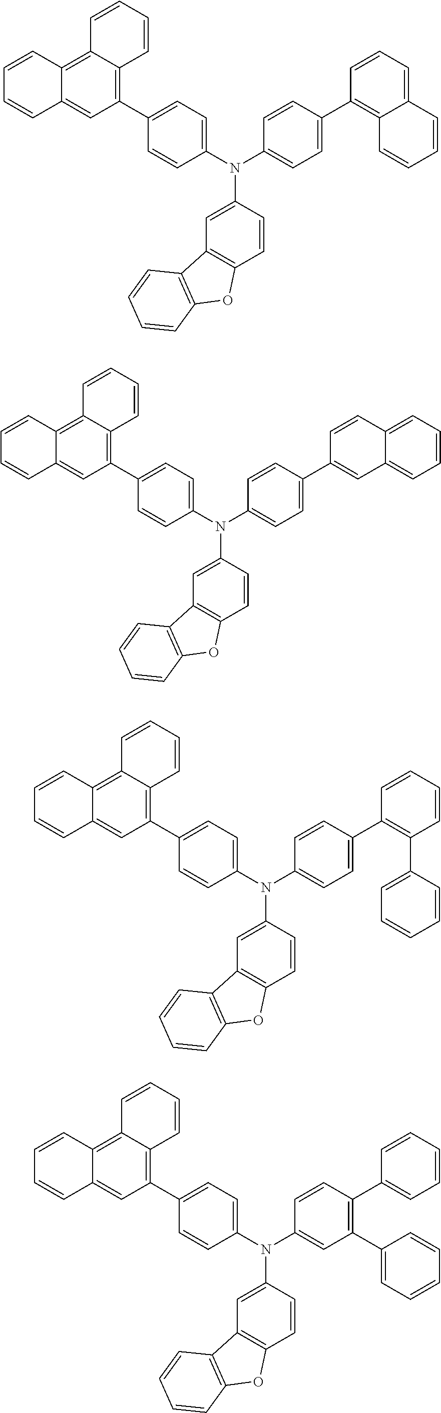

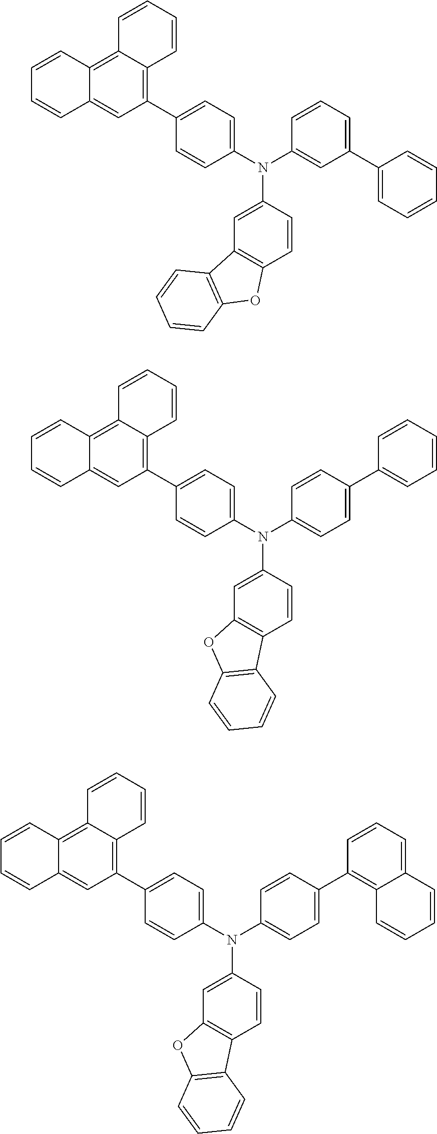 [SMILES: c1ccc(-c2cccc(N(c3ccc(-c4cc5ccccc5c5ccccc45)cc3)c3ccc4oc5ccccc5c4c3)c2)cc1 ; c1ccc(-c2ccc(N(c3ccc(-c4cc5ccccc5c5ccccc45)cc3)c3ccc4c(c3)oc3ccccc34)cc2)cc1 ; c1ccc2c(-c3ccc(N(c4ccc(-c5cc6ccccc6c6ccccc56)cc4)c4ccc5c(c4)oc4ccccc45)cc3)cccc2c1]

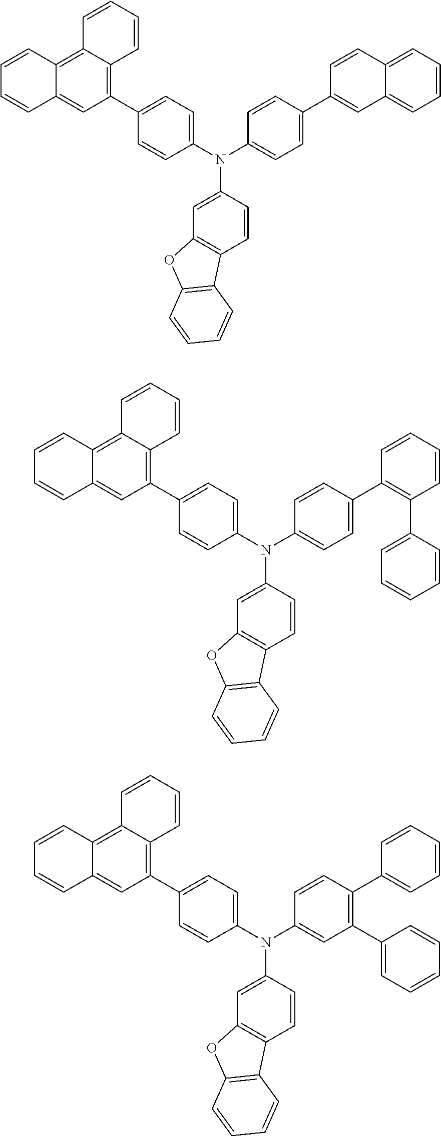 [SMILES: c1ccc2cc(-c3ccc(N(c4ccc(-c5cc6ccccc6c6ccccc56)cc4)c4ccc5c(c4)oc4ccccc45)cc3)ccc2c1 ; c1ccc(-c2ccccc2-c2ccc(N(c3ccc(-c4cc5ccccc5c5ccccc45)cc3)c3ccc4c(c3)oc3ccccc34)cc2)cc1 ; c1ccc(-c2ccc(N(c3ccc(-c4cc5ccccc5c5ccccc45)cc3)c3ccc4c(c3)oc3ccccc34)cc2-c2ccccc2)cc1]

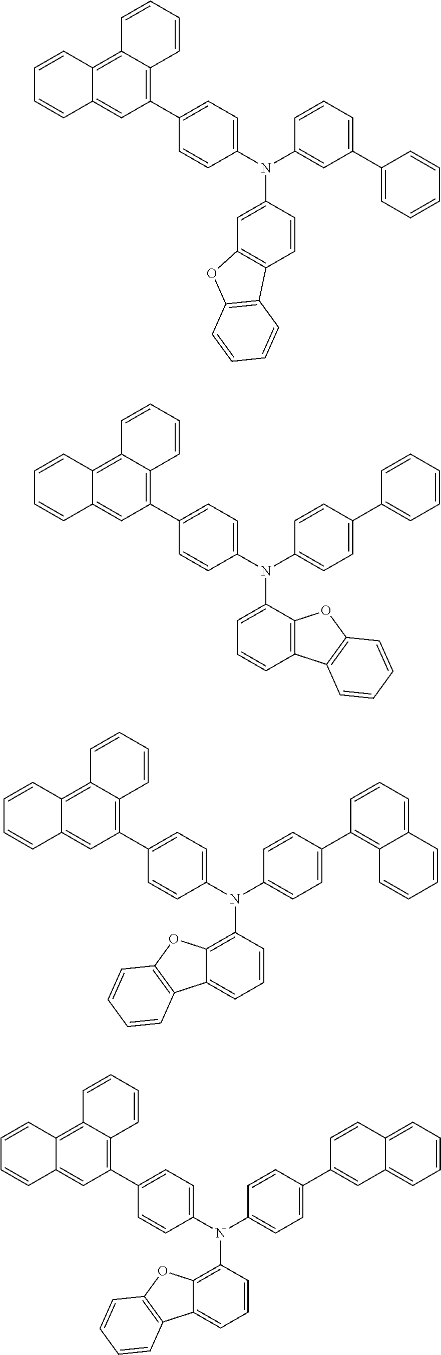 [SMILES: c1ccc(-c2cccc(N(c3ccc(-c4cc5ccccc5c5ccccc45)cc3)c3ccc4c(c3)oc3ccccc34)c2)cc1 ; c1ccc(-c2ccc(N(c3ccc(-c4cc5ccccc5c5ccccc45)cc3)c3cccc4c3oc3ccccc34)cc2)cc1 ; c1ccc2c(-c3ccc(N(c4ccc(-c5cc6ccccc6c6ccccc56)cc4)c4cccc5c4oc4ccccc45)cc3)cccc2c1 ; c1ccc2cc(-c3ccc(N(c4ccc(-c5cc6ccccc6c6ccccc56)cc4)c4cccc5c4oc4ccccc45)cc3)ccc2c1]

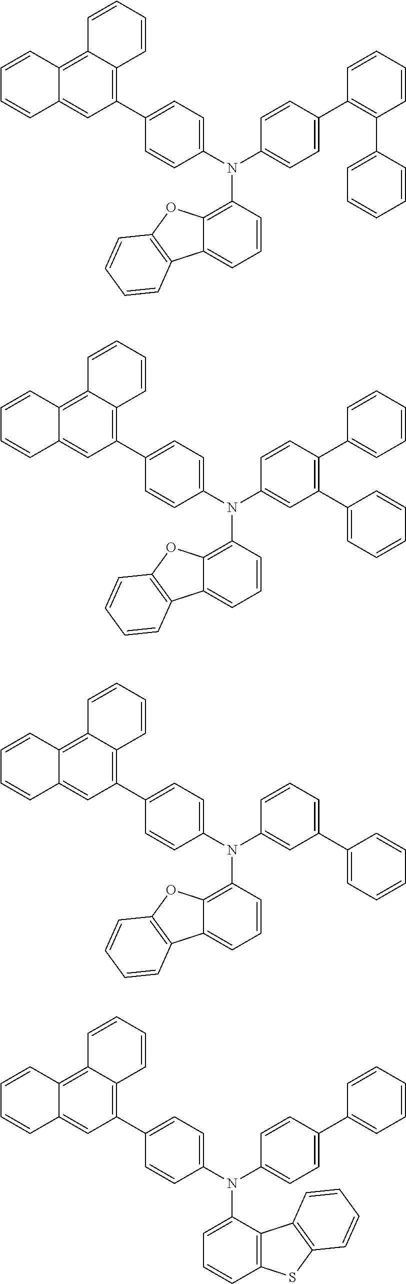

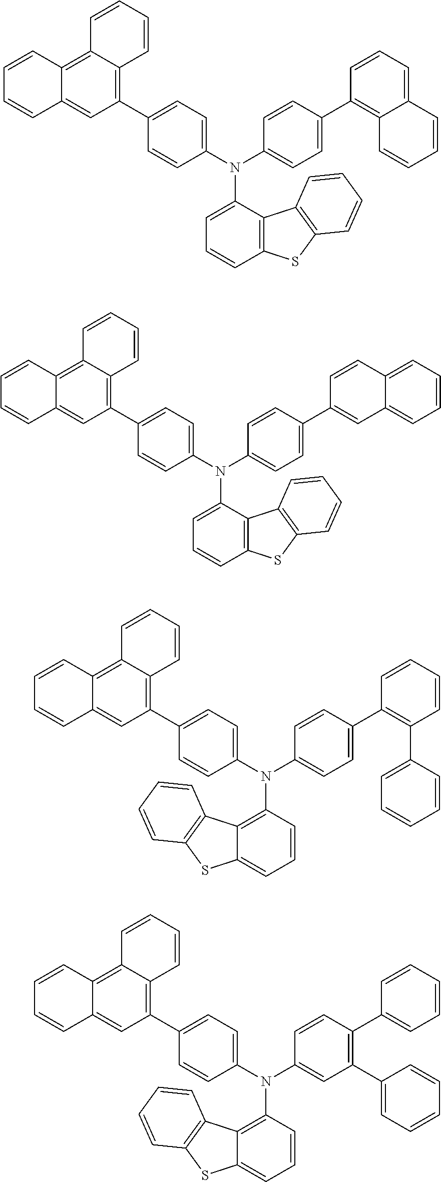 [SMILES: c1ccc2c(-c3ccc(N(c4ccc(-c5cc6ccccc6c6ccccc56)cc4)c4cccc5sc6ccccc6c45)cc3)cccc2c1 ; c1ccc2cc(-c3ccc(N(c4ccc(-c5cc6ccccc6c6ccccc56)cc4)c4cccc5sc6ccccc6c45)cc3)ccc2c1 ; c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)cc2-c2ccccc2)cc1]

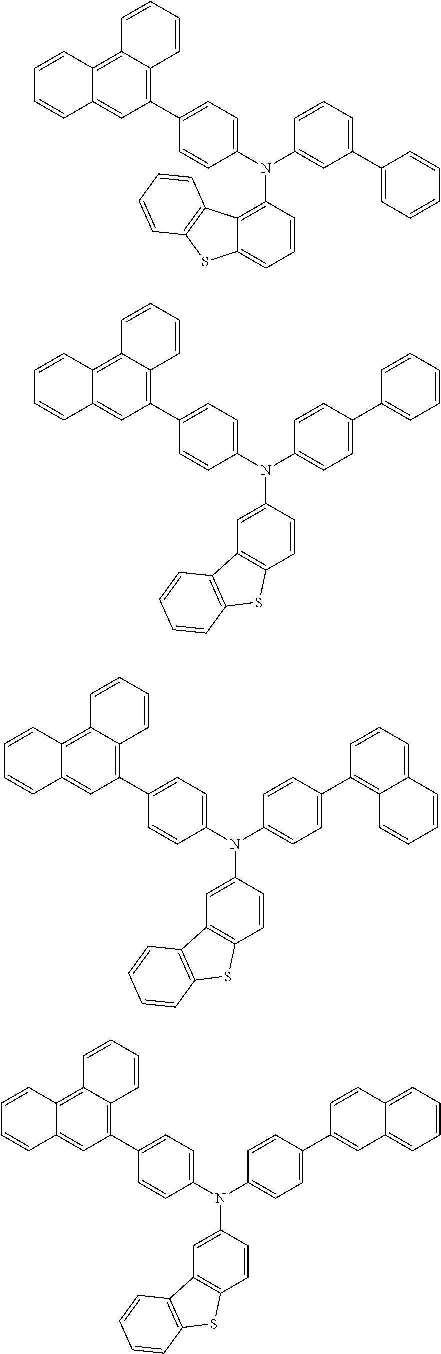 [SMILES: c1ccc(-c2cccc(N(c3ccc(-c4cc5ccccc5c5ccccc45)cc3)c3cccc4sc5ccccc5c34)c2)cc1 ; c1ccc(-c2ccc(N(c3ccc(-c4cc5ccccc5c5ccccc45)cc3)c3ccc4sc5ccccc5c4c3)cc2)cc1 ; c1ccc2c(-c3ccc(N(c4ccc(-c5cc6ccccc6c6ccccc56)cc4)c4ccc5sc6ccccc6c5c4)cc3)cccc2c1 ; c1ccc2cc(-c3ccc(N(c4ccc(-c5cc6ccccc6c6ccccc56)cc4)c4ccc5sc6ccccc6c5c4)cc3)ccc2c1]

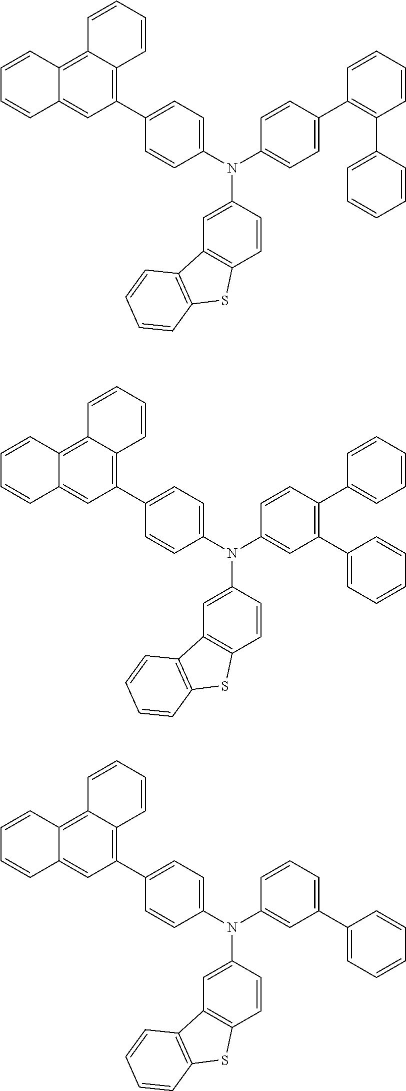 [SMILES: c1ccc(-c2ccccc2-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4sc5ccccc5c4c3)cc2-c2ccccc2)cc1 ; c1ccc(-c2cccc(N(c3ccc(-c4cc5ccccc5c5ccccc45)cc3)c3ccc4sc5ccccc5c4c3)c2)cc1]

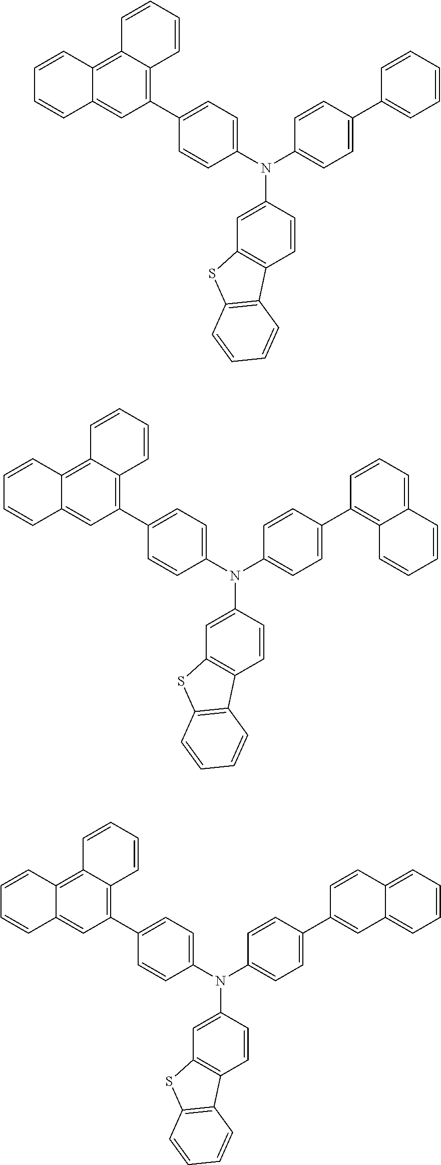 [SMILES: c1ccc(-c2ccc(N(c3ccc(-c4cc5ccccc5c5ccccc45)cc3)c3ccc4c(c3)sc3ccccc34)cc2)cc1 ; c1ccc2c(-c3ccc(N(c4ccc(-c5cc6ccccc6c6ccccc56)cc4)c4ccc5c(c4)sc4ccccc45)cc3)cccc2c1 ; c1ccc2cc(-c3ccc(N(c4ccc(-c5cc6ccccc6c6ccccc56)cc4)c4ccc5c(c4)sc4ccccc45)cc3)ccc2c1]

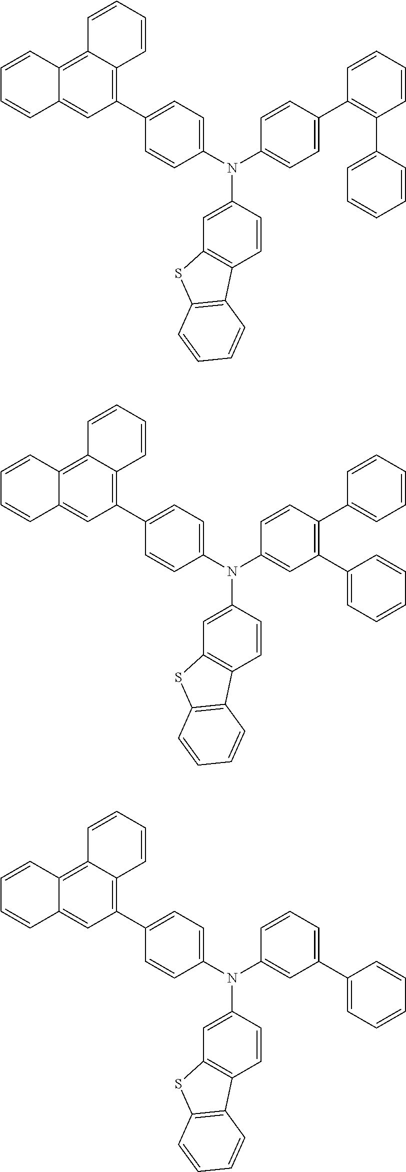 [SMILES: c1ccc(-c2ccccc2-c2ccc(N(c3ccc(-c4cc5ccccc5c5ccccc45)cc3)c3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2-c2ccccc2)cc1 ; c1ccc(-c2cccc(N(c3ccc(-c4cc5ccccc5c5ccccc45)cc3)c3ccc4c(c3)sc3ccccc34)c2)cc1]

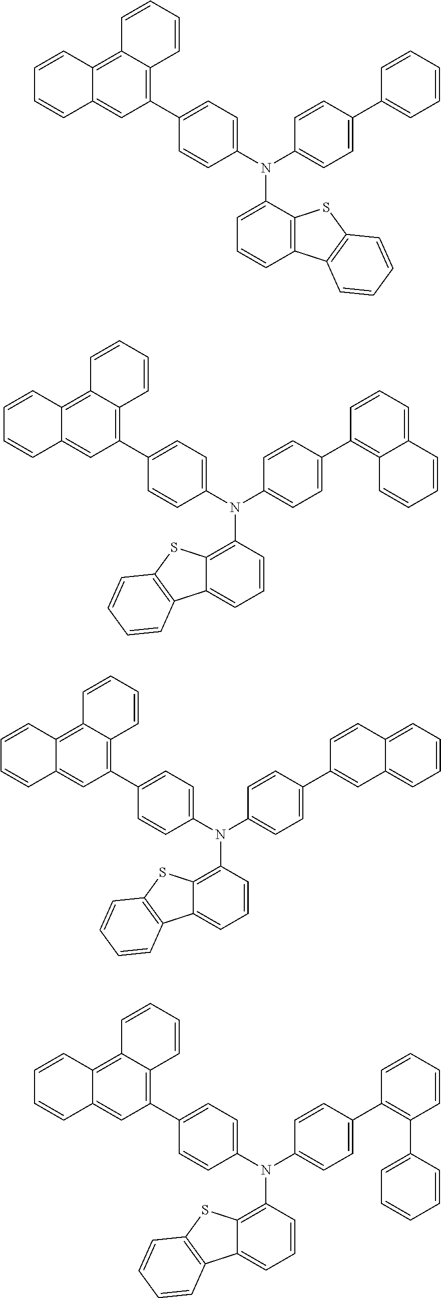

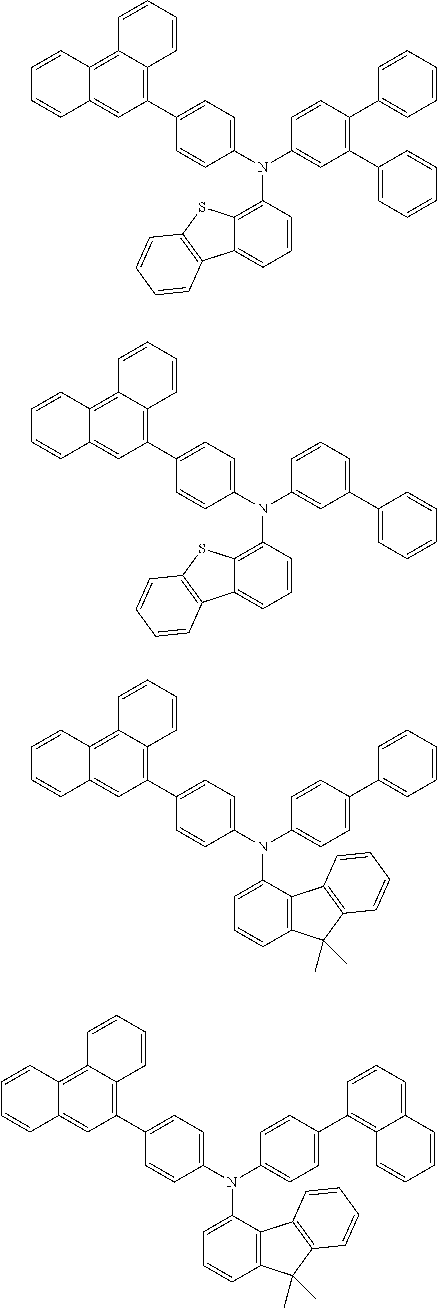

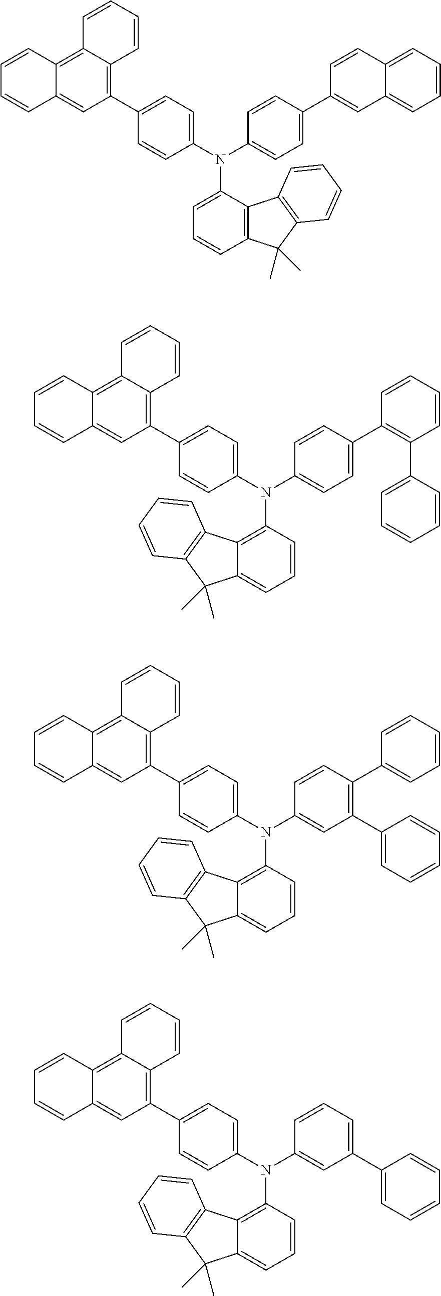 [SMILES: CC1(C)c2ccccc2-c2c(N(c3ccc(-c4ccc5ccccc5c4)cc3)c3ccc(-c4cc5ccccc5c5ccccc45)cc3)cccc21 ; CC1(C)c2ccccc2-c2c(N(c3ccc(-c4ccccc4-c4ccccc4)cc3)c3ccc(-c4cc5ccccc5c5ccccc45)cc3)cccc21 ; CC1(C)c2ccccc2-c2c(N(c3ccc(-c4cc5ccccc5c5ccccc45)cc3)c3ccc(-c4ccccc4)c(-c4ccccc4)c3)cccc21 ; CC1(C)c2ccccc2-c2c(N(c3ccc(-c4cc5ccccc5c5ccccc45)cc3)c3cccc(-c4ccccc4)c3)cccc21]

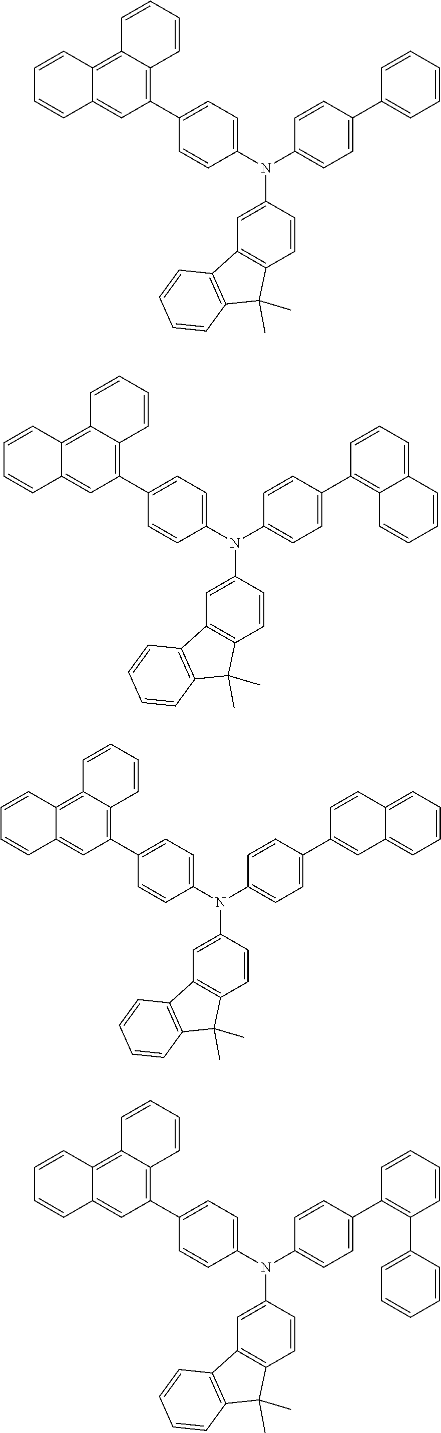

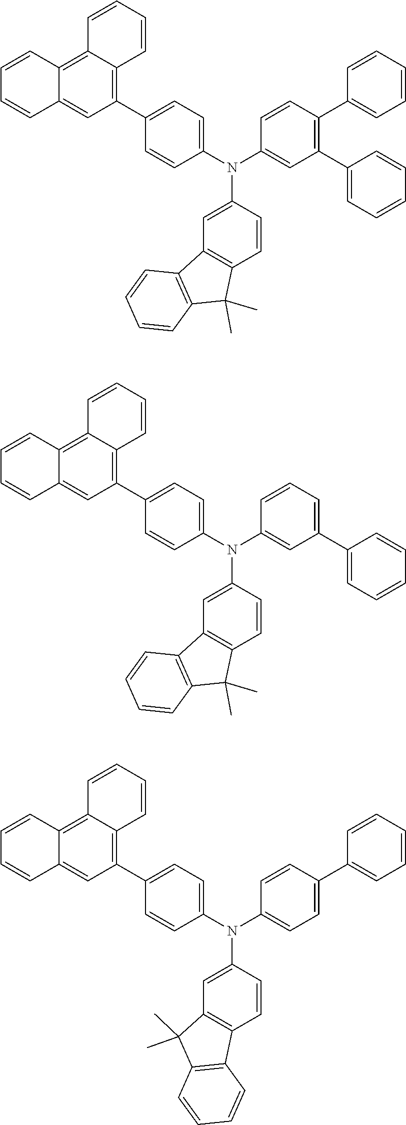 [SMILES: CC1(C)c2ccccc2-c2cc(N(c3ccc(-c4cc5ccccc5c5ccccc45)cc3)c3ccc(-c4ccccc4)c(-c4ccccc4)c3)ccc21 ; CC1(C)c2ccccc2-c2cc(N(c3ccc(-c4cc5ccccc5c5ccccc45)cc3)c3cccc(-c4ccccc4)c3)ccc21 ; CC1(C)c2ccccc2-c2ccc(N(c3ccc(-c4ccccc4)cc3)c3ccc(-c4cc5ccccc5c5ccccc45)cc3)cc21]

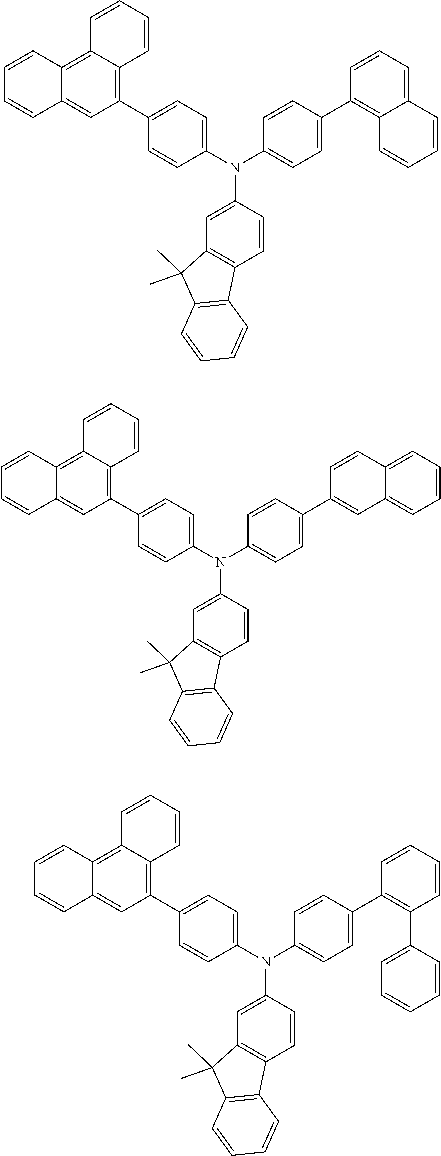 [SMILES: CC1(C)c2ccccc2-c2ccc(N(c3ccc(-c4cccc5ccccc45)cc3)c3ccc(-c4cc5ccccc5c5ccccc45)cc3)cc21 ; CC1(C)c2ccccc2-c2ccc(N(c3ccc(-c4ccc5ccccc5c4)cc3)c3ccc(-c4cc5ccccc5c5ccccc45)cc3)cc21 ; CC1(C)c2ccccc2-c2ccc(N(c3ccc(-c4ccccc4-c4ccccc4)cc3)c3ccc(-c4cc5ccccc5c5ccccc45)cc3)cc21]

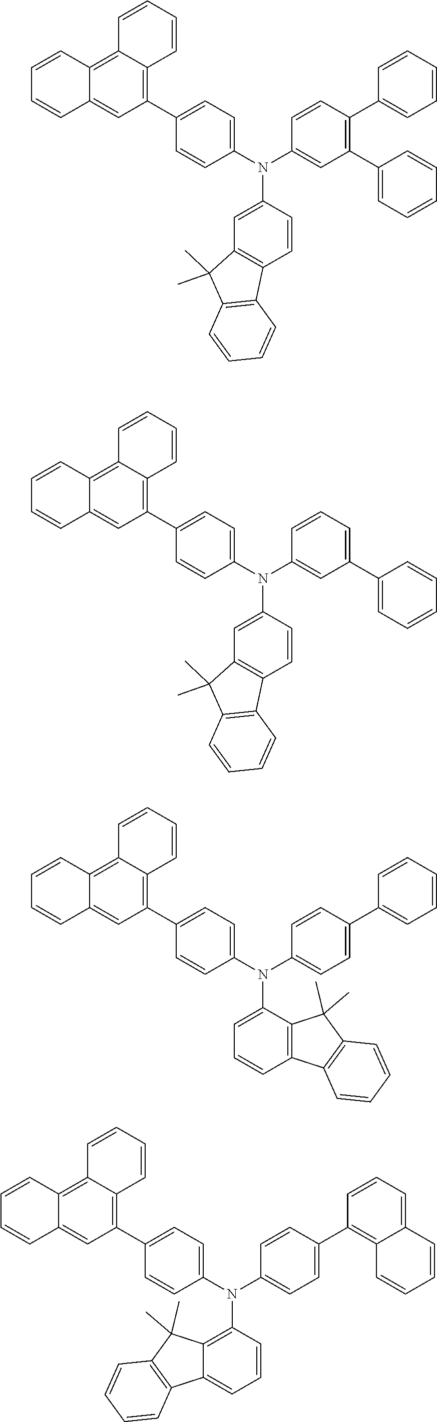 [SMILES: CC1(C)c2ccccc2-c2ccc(N(c3ccc(-c4cc5ccccc5c5ccccc45)cc3)c3ccc(-c4ccccc4)c(-c4ccccc4)c3)cc21 ; CC1(C)c2ccccc2-c2ccc(N(c3ccc(-c4cc5ccccc5c5ccccc45)cc3)c3cccc(-c4ccccc4)c3)cc21 ; CC1(C)c2ccccc2-c2cccc(N(c3ccc(-c4ccccc4)cc3)c3ccc(-c4cc5ccccc5c5ccccc45)cc3)c21 ; CC1(C)c2ccccc2-c2cccc(N(c3ccc(-c4cccc5ccccc45)cc3)c3ccc(-c4cc5ccccc5c5ccccc45)cc3)c21]

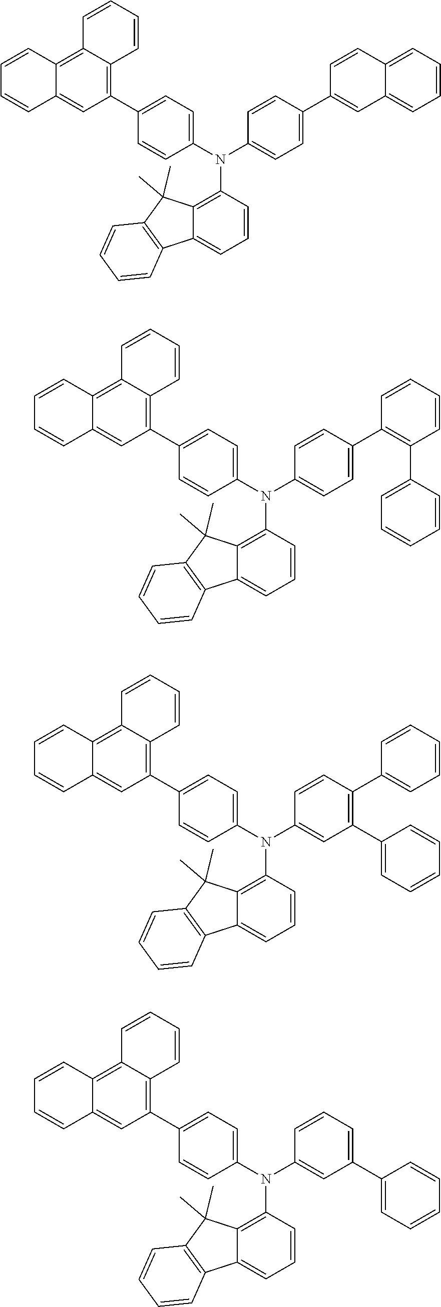

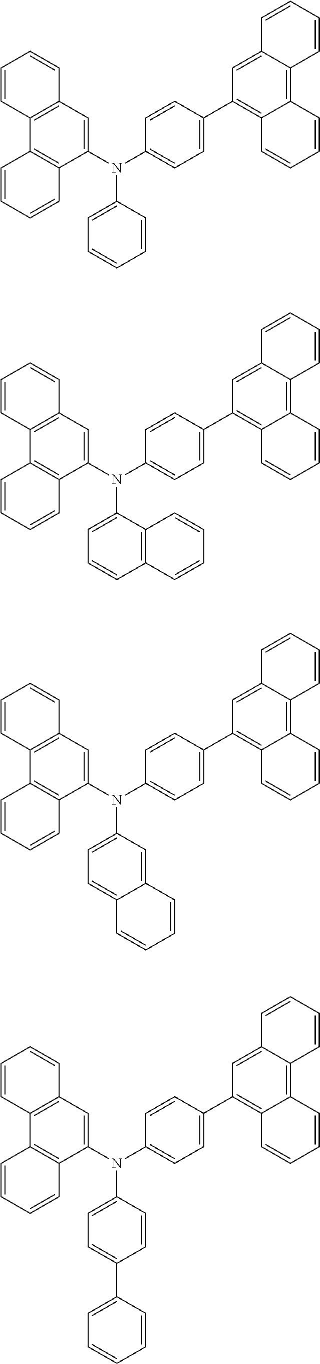 [SMILES: c1ccc(N(c2ccc(-c3cc4ccccc4c4ccccc34)cc2)c2cc3ccccc3c3ccccc23)cc1 ; c1ccc2c(N(c3ccc(-c4cc5ccccc5c5ccccc45)cc3)c3cc4ccccc4c4ccccc34)cccc2c1 ; c1ccc2cc(N(c3ccc(-c4cc5ccccc5c5ccccc45)cc3)c3cc4ccccc4c4ccccc34)ccc2c1 ; c1ccc(-c2ccc(N(c3ccc(-c4cc5ccccc5c5ccccc45)cc3)c3cc4ccccc4c4ccccc34)cc2)cc1]

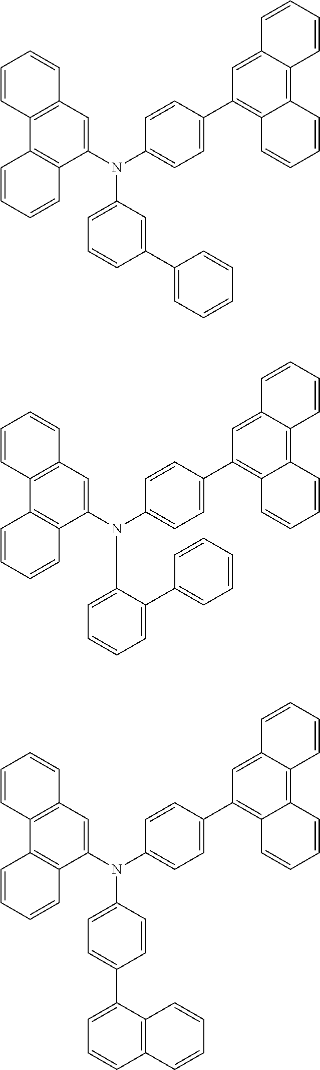 [SMILES: c1ccc(-c2cccc(N(c3ccc(-c4cc5ccccc5c5ccccc45)cc3)c3cc4ccccc4c4ccccc34)c2)cc1 ; c1ccc(-c2ccccc2N(c2ccc(-c3cc4ccccc4c4ccccc34)cc2)c2cc3ccccc3c3ccccc23)cc1 ; c1ccc2c(-c3ccc(N(c4ccc(-c5cc6ccccc6c6ccccc56)cc4)c4cc5ccccc5c5ccccc45)cc3)cccc2c1]

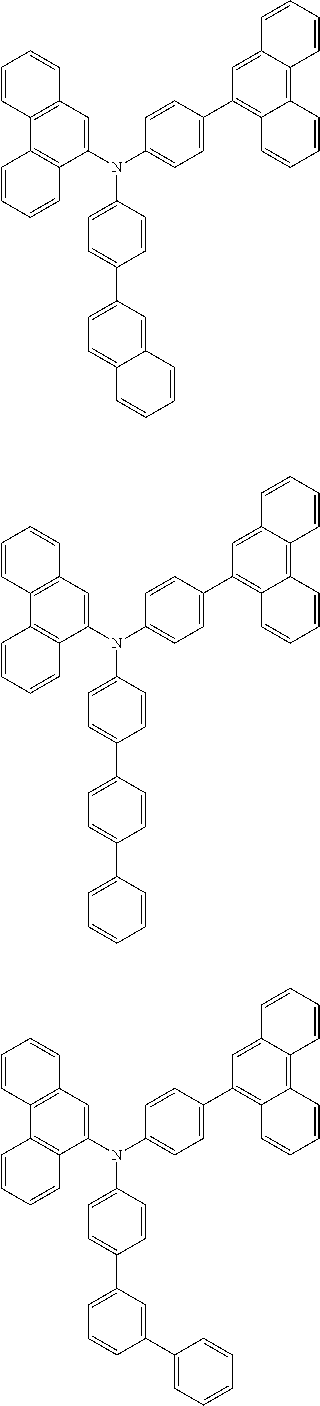 [SMILES: c1ccc2cc(-c3ccc(N(c4ccc(-c5cc6ccccc6c6ccccc56)cc4)c4cc5ccccc5c5ccccc45)cc3)ccc2c1 ; c1ccc(-c2ccc(-c3ccc(N(c4ccc(-c5cc6ccccc6c6ccccc56)cc4)c4cc5ccccc5c5ccccc45)cc3)cc2)cc1 ; c1ccc(-c2cccc(-c3ccc(N(c4ccc(-c5cc6ccccc6c6ccccc56)cc4)c4cc5ccccc5c5ccccc45)cc3)c2)cc1]

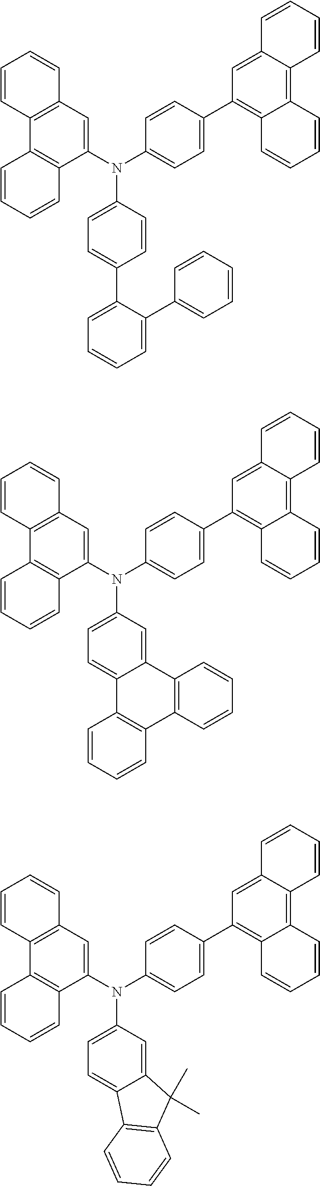 [SMILES: c1ccc(-c2ccccc2-c2ccc(N(c3ccc(-c4cc5ccccc5c5ccccc45)cc3)c3cc4ccccc4c4ccccc34)cc2)cc1 ; c1ccc2c(c1)cc(-c1ccc(N(c3ccc4c5ccccc5c5ccccc5c4c3)c3cc4ccccc4c4ccccc34)cc1)c1ccccc12 ; CC1(C)c2ccccc2-c2ccc(N(c3ccc(-c4cc5ccccc5c5ccccc45)cc3)c3cc4ccccc4c4ccccc34)cc21]

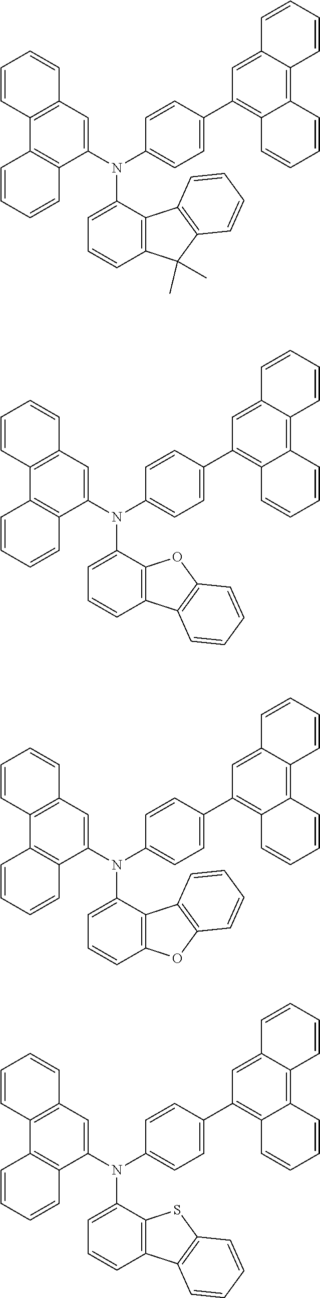 [SMILES: CC1(C)c2ccccc2-c2c(N(c3ccc(-c4cc5ccccc5c5ccccc45)cc3)c3cc4ccccc4c4ccccc34)cccc21 ; c1ccc2c(c1)cc(-c1ccc(N(c3cc4ccccc4c4ccccc34)c3cccc4c3oc3ccccc34)cc1)c1ccccc12 ; c1ccc2c(c1)cc(-c1ccc(N(c3cc4ccccc4c4ccccc34)c3cccc4oc5ccccc5c34)cc1)c1ccccc12 ; c1ccc2c(c1)cc(-c1ccc(N(c3cc4ccccc4c4ccccc34)c3cccc4c3sc3ccccc34)cc1)c1ccccc12]

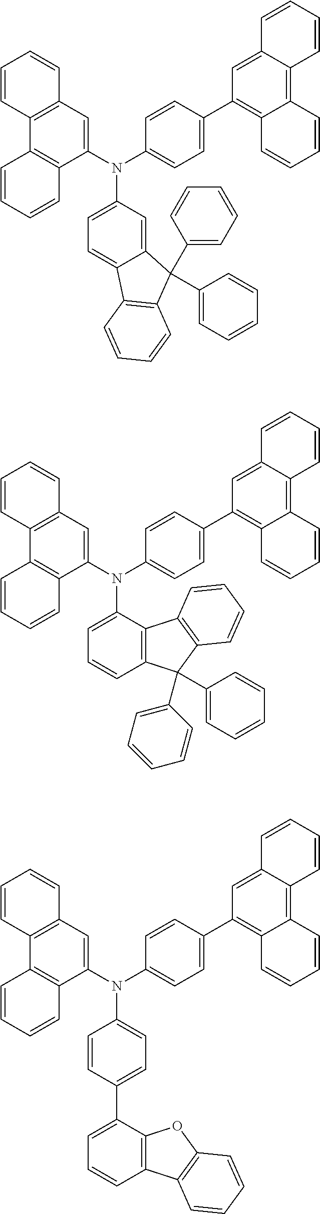 [SMILES: c1ccc(C2(c3ccccc3)c3ccccc3-c3ccc(N(c4ccc(-c5cc6ccccc6c6ccccc56)cc4)c4cc5ccccc5c5ccccc45)cc32)cc1 ; c1ccc(C2(c3ccccc3)c3ccccc3-c3c(N(c4ccc(-c5cc6ccccc6c6ccccc56)cc4)c4cc5ccccc5c5ccccc45)cccc32)cc1 ; c1ccc2c(c1)cc(-c1ccc(N(c3ccc(-c4cccc5c4oc4ccccc45)cc3)c3cc4ccccc4c4ccccc34)cc1)c1ccccc12]

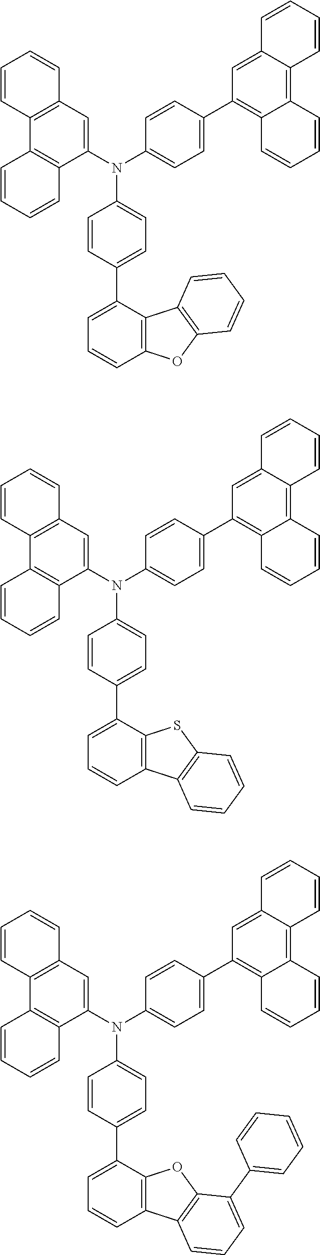 [SMILES: c1ccc2c(c1)cc(-c1ccc(N(c3ccc(-c4cccc5oc6ccccc6c45)cc3)c3cc4ccccc4c4ccccc34)cc1)c1ccccc12 ; c1ccc2c(c1)cc(-c1ccc(N(c3ccc(-c4cccc5c4sc4ccccc45)cc3)c3cc4ccccc4c4ccccc34)cc1)c1ccccc12 ; c1ccc(-c2cccc3c2oc2c(-c4ccc(N(c5ccc(-c6cc7ccccc7c7ccccc67)cc5)c5cc6ccccc6c6ccccc56)cc4)cccc23)cc1]

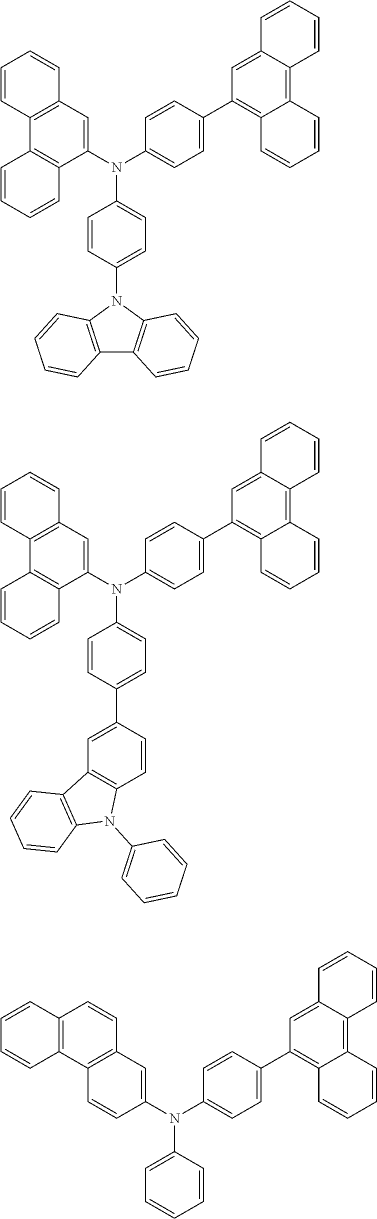 [SMILES: c1ccc2c(c1)cc(-c1ccc(N(c3ccc(-n4c5ccccc5c5ccccc54)cc3)c3cc4ccccc4c4ccccc34)cc1)c1ccccc12 ; c1ccc(-n2c3ccccc3c3cc(-c4ccc(N(c5ccc(-c6cc7ccccc7c7ccccc67)cc5)c5cc6ccccc6c6ccccc56)cc4)ccc32)cc1 ; c1ccc(N(c2ccc(-c3cc4ccccc4c4ccccc34)cc2)c2ccc3c(ccc4ccccc43)c2)cc1]

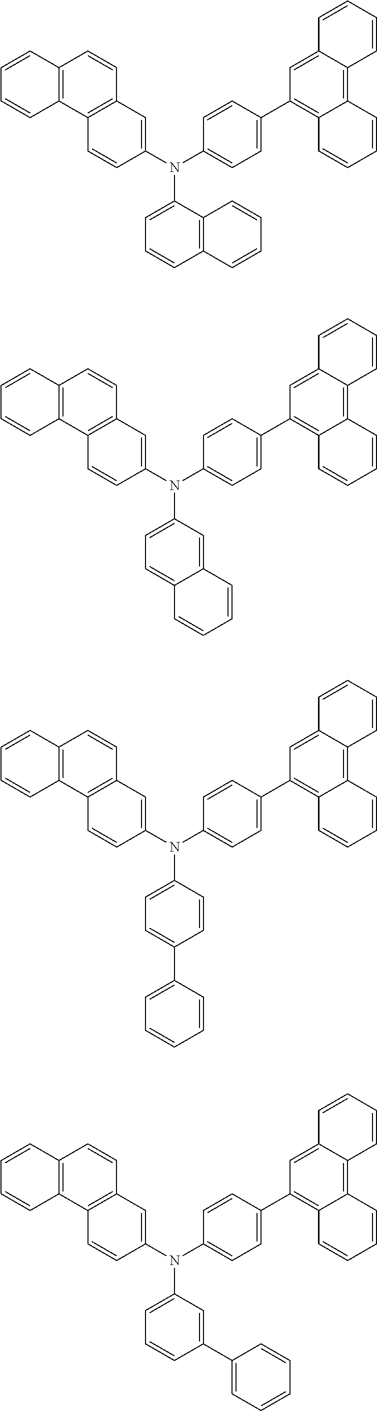 [SMILES: c1ccc2c(N(c3ccc(-c4cc5ccccc5c5ccccc45)cc3)c3ccc4c(ccc5ccccc54)c3)cccc2c1 ; c1ccc2cc(N(c3ccc(-c4cc5ccccc5c5ccccc45)cc3)c3ccc4c(ccc5ccccc54)c3)ccc2c1 ; c1ccc(-c2ccc(N(c3ccc(-c4cc5ccccc5c5ccccc45)cc3)c3ccc4c(ccc5ccccc54)c3)cc2)cc1 ; c1ccc(-c2cccc(N(c3ccc(-c4cc5ccccc5c5ccccc45)cc3)c3ccc4c(ccc5ccccc54)c3)c2)cc1]

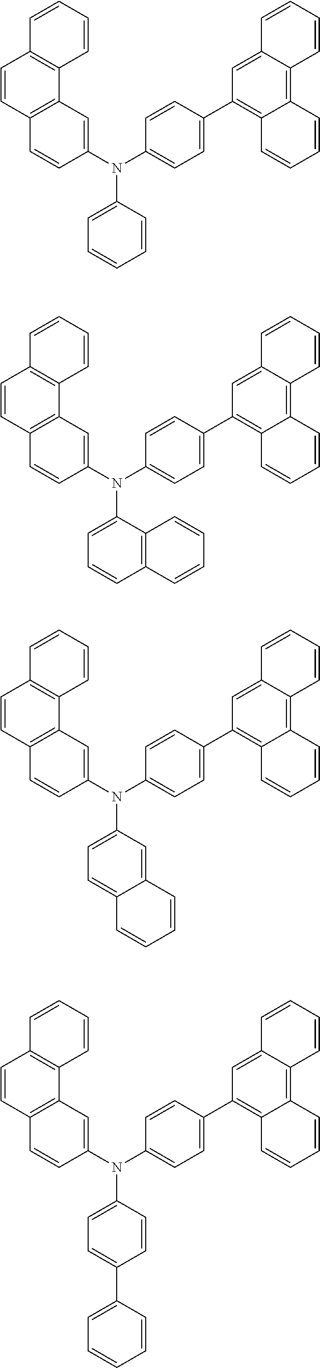 [SMILES: c1ccc(N(c2ccc(-c3cc4ccccc4c4ccccc34)cc2)c2ccc3ccc4ccccc4c3c2)cc1 ; c1ccc2c(N(c3ccc(-c4cc5ccccc5c5ccccc45)cc3)c3ccc4ccc5ccccc5c4c3)cccc2c1 ; c1ccc2cc(N(c3ccc(-c4cc5ccccc5c5ccccc45)cc3)c3ccc4ccc5ccccc5c4c3)ccc2c1 ; c1ccc(-c2ccc(N(c3ccc(-c4cc5ccccc5c5ccccc45)cc3)c3ccc4ccc5ccccc5c4c3)cc2)cc1]

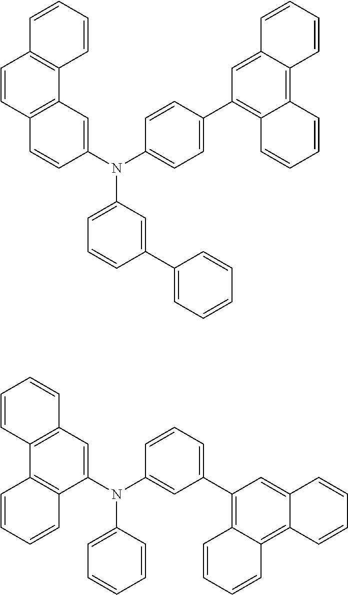 [SMILES: c1ccc(-c2cccc(N(c3ccc(-c4cc5ccccc5c5ccccc45)cc3)c3ccc4ccc5ccccc5c4c3)c2)cc1 ; c1ccc(N(c2cccc(-c3cc4ccccc4c4ccccc34)c2)c2cc3ccccc3c3ccccc23)cc1]

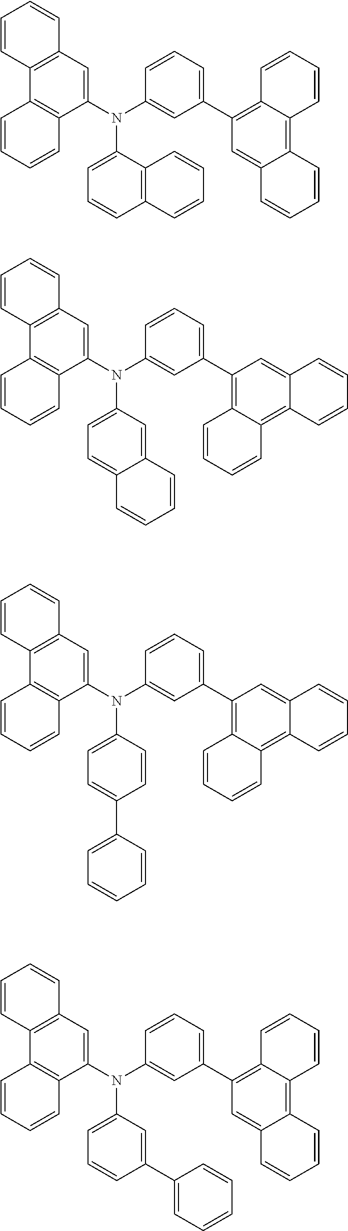 [SMILES: c1cc(-c2cc3ccccc3c3ccccc23)cc(N(c2cccc3ccccc23)c2cc3ccccc3c3ccccc23)c1 ; c1cc(-c2cc3ccccc3c3ccccc23)cc(N(c2ccc3ccccc3c2)c2cc3ccccc3c3ccccc23)c1 ; c1ccc(-c2ccc(N(c3cccc(-c4cc5ccccc5c5ccccc45)c3)c3cc4ccccc4c4ccccc34)cc2)cc1 ; c1ccc(-c2cccc(N(c3cccc(-c4cc5ccccc5c5ccccc45)c3)c3cc4ccccc4c4ccccc34)c2)cc1]

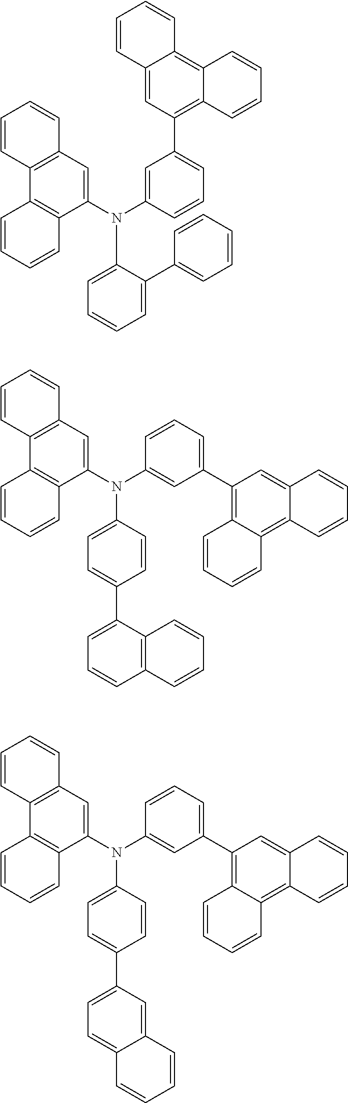 [SMILES: c1ccc(-c2ccccc2N(c2cccc(-c3cc4ccccc4c4ccccc34)c2)c2cc3ccccc3c3ccccc23)cc1 ; c1cc(-c2cc3ccccc3c3ccccc23)cc(N(c2ccc(-c3cccc4ccccc34)cc2)c2cc3ccccc3c3ccccc23)c1 ; c1cc(-c2cc3ccccc3c3ccccc23)cc(N(c2ccc(-c3ccc4ccccc4c3)cc2)c2cc3ccccc3c3ccccc23)c1]

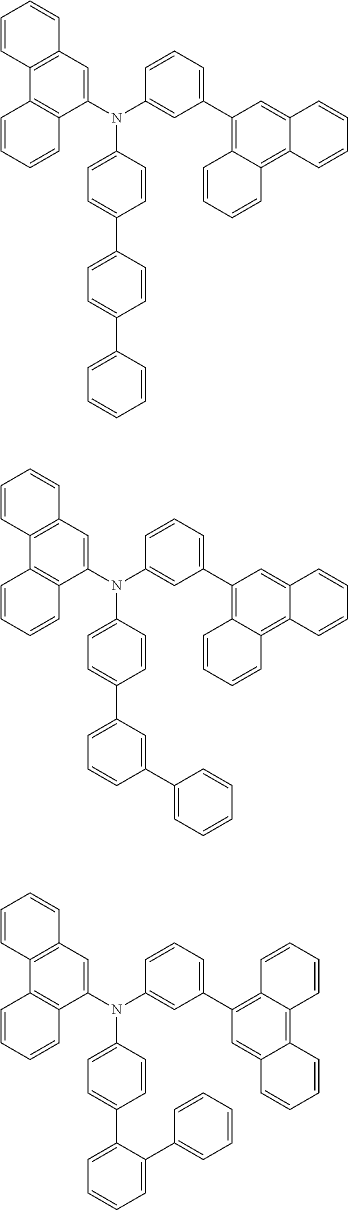 [SMILES: c1ccc(-c2ccc(-c3ccc(N(c4cccc(-c5cc6ccccc6c6ccccc56)c4)c4cc5ccccc5c5ccccc45)cc3)cc2)cc1 ; c1ccc(-c2cccc(-c3ccc(N(c4cccc(-c5cc6ccccc6c6ccccc56)c4)c4cc5ccccc5c5ccccc45)cc3)c2)cc1 ; c1ccc(-c2ccccc2-c2ccc(N(c3cccc(-c4cc5ccccc5c5ccccc45)c3)c3cc4ccccc4c4ccccc34)cc2)cc1]

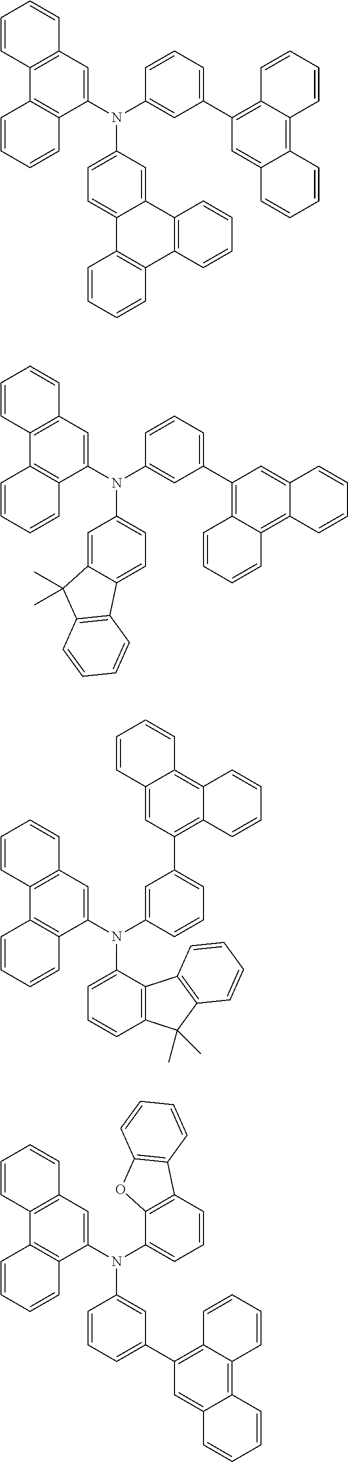

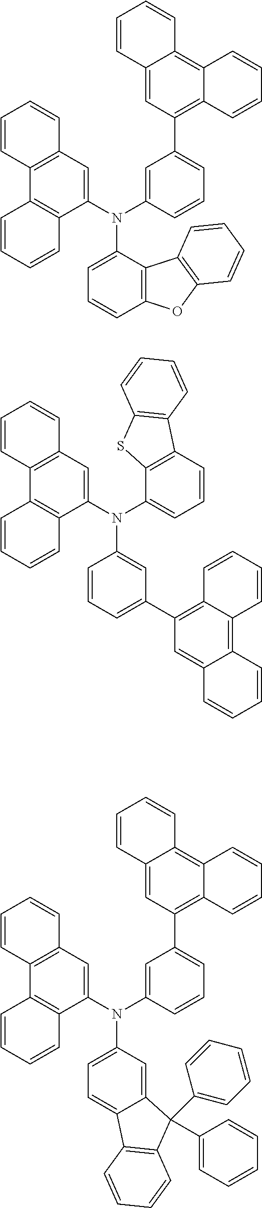

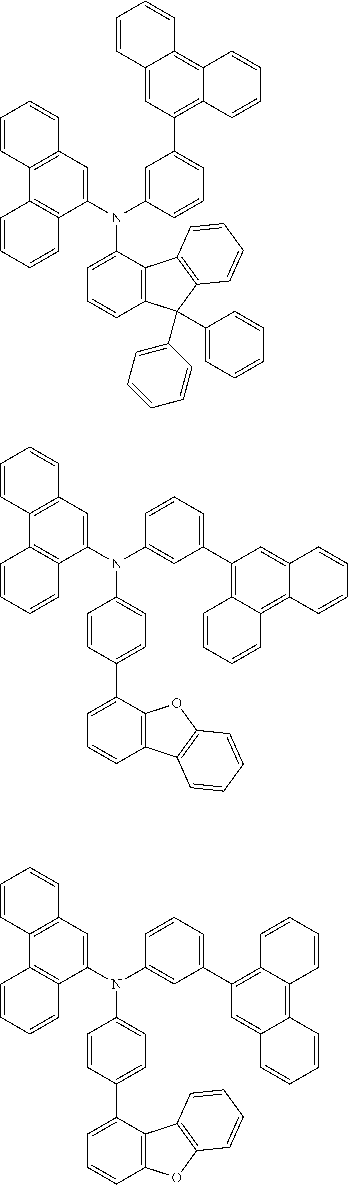 [SMILES: c1ccc(C2(c3ccccc3)c3ccccc3-c3c(N(c4cccc(-c5cc6ccccc6c6ccccc56)c4)c4cc5ccccc5c5ccccc45)cccc32)cc1 ; c1cc(-c2cc3ccccc3c3ccccc23)cc(N(c2ccc(-c3cccc4c3oc3ccccc34)cc2)c2cc3ccccc3c3ccccc23)c1 ; c1cc(-c2cc3ccccc3c3ccccc23)cc(N(c2ccc(-c3cccc4oc5ccccc5c34)cc2)c2cc3ccccc3c3ccccc23)c1]

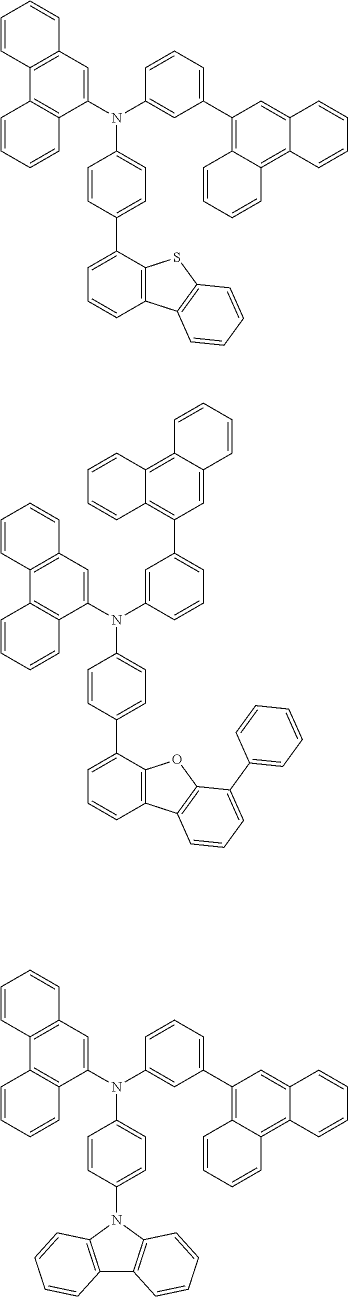 [SMILES: c1cc(-c2cc3ccccc3c3ccccc23)cc(N(c2ccc(-c3cccc4c3sc3ccccc34)cc2)c2cc3ccccc3c3ccccc23)c1 ; c1ccc(-c2cccc3c2oc2c(-c4ccc(N(c5cccc(-c6cc7ccccc7c7ccccc67)c5)c5cc6ccccc6c6ccccc56)cc4)cccc23)cc1 ; c1cc(-c2cc3ccccc3c3ccccc23)cc(N(c2ccc(-n3c4ccccc4c4ccccc43)cc2)c2cc3ccccc3c3ccccc23)c1]

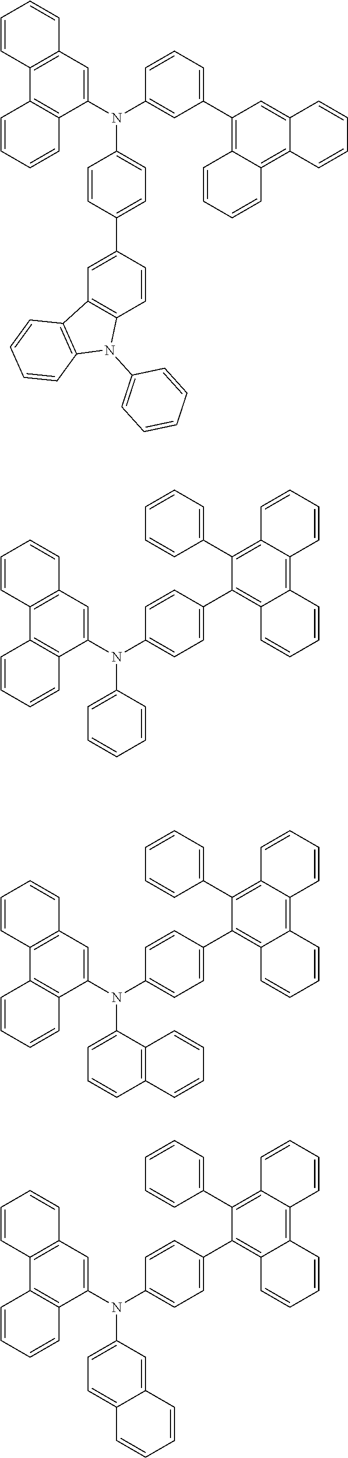

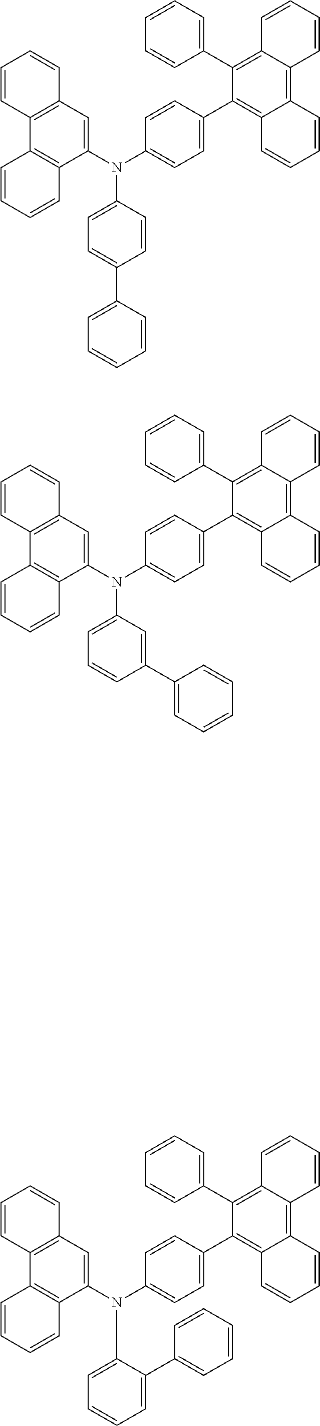 [SMILES: c1ccc(-c2ccc(N(c3ccc(-c4c(-c5ccccc5)c5ccccc5c5ccccc45)cc3)c3cc4ccccc4c4ccccc34)cc2)cc1 ; c1ccc(-c2cccc(N(c3ccc(-c4c(-c5ccccc5)c5ccccc5c5ccccc45)cc3)c3cc4ccccc4c4ccccc34)c2)cc1 ; c1ccc(-c2ccccc2N(c2ccc(-c3c(-c4ccccc4)c4ccccc4c4ccccc34)cc2)c2cc3ccccc3c3ccccc23)cc1]

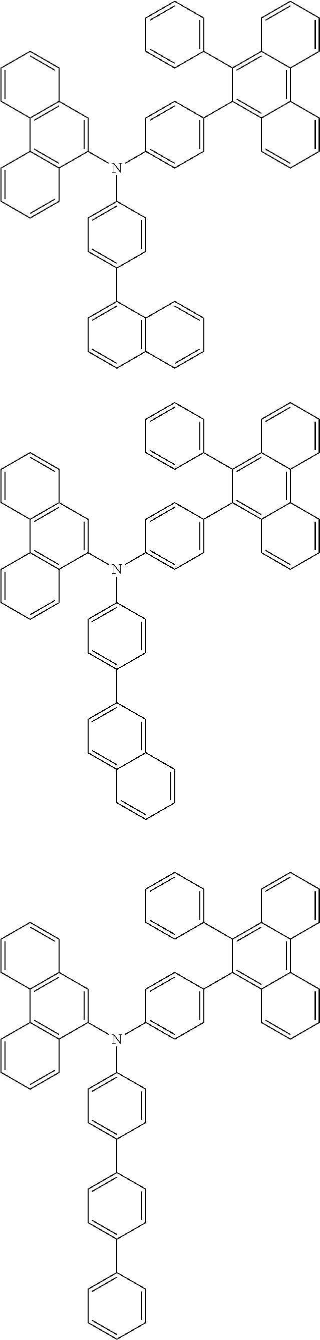

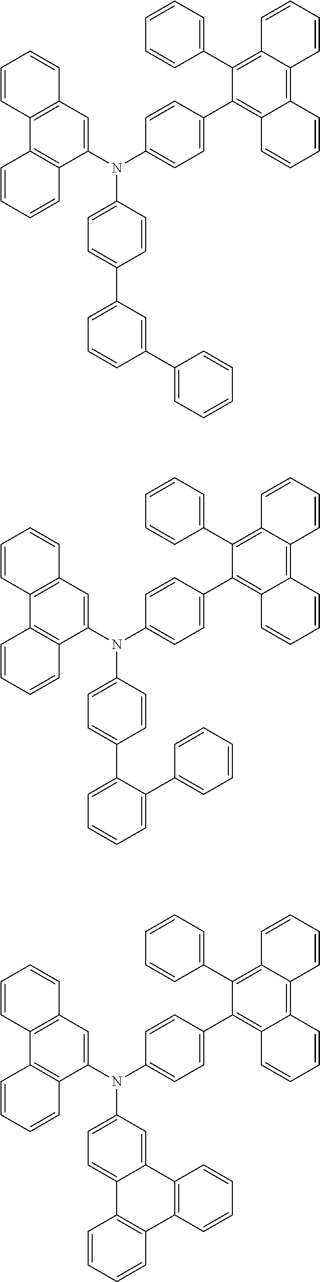 [SMILES: c1ccc(-c2cccc(-c3ccc(N(c4ccc(-c5c(-c6ccccc6)c6ccccc6c6ccccc56)cc4)c4cc5ccccc5c5ccccc45)cc3)c2)cc1 ; c1ccc(-c2ccccc2-c2ccc(N(c3ccc(-c4c(-c5ccccc5)c5ccccc5c5ccccc45)cc3)c3cc4ccccc4c4ccccc34)cc2)cc1 ; c1ccc(-c2c(-c3ccc(N(c4ccc5c6ccccc6c6ccccc6c5c4)c4cc5ccccc5c5ccccc45)cc3)c3ccccc3c3ccccc23)cc1]

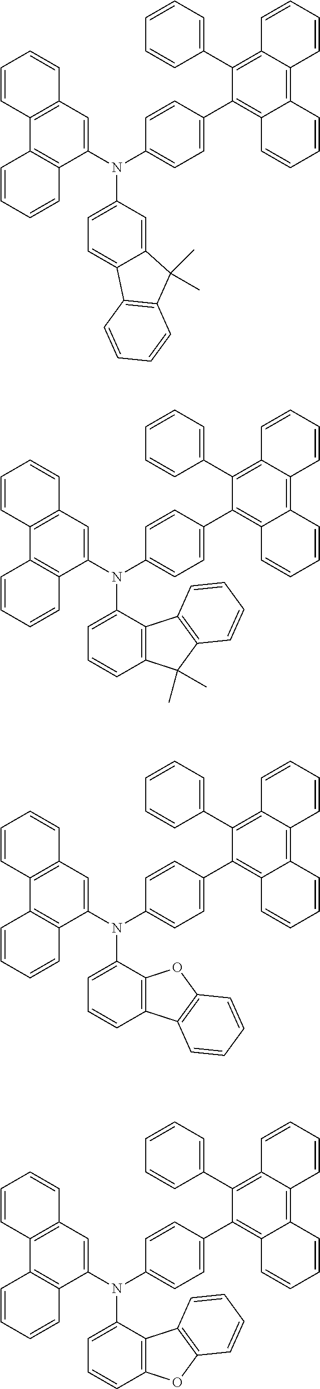 [SMILES: CC1(C)c2ccccc2-c2ccc(N(c3ccc(-c4c(-c5ccccc5)c5ccccc5c5ccccc45)cc3)c3cc4ccccc4c4ccccc34)cc21 ; CC1(C)c2ccccc2-c2c(N(c3ccc(-c4c(-c5ccccc5)c5ccccc5c5ccccc45)cc3)c3cc4ccccc4c4ccccc34)cccc21 ; c1ccc(-c2c(-c3ccc(N(c4cc5ccccc5c5ccccc45)c4cccc5c4oc4ccccc45)cc3)c3ccccc3c3ccccc23)cc1 ; c1ccc(-c2c(-c3ccc(N(c4cc5ccccc5c5ccccc45)c4cccc5oc6ccccc6c45)cc3)c3ccccc3c3ccccc23)cc1]

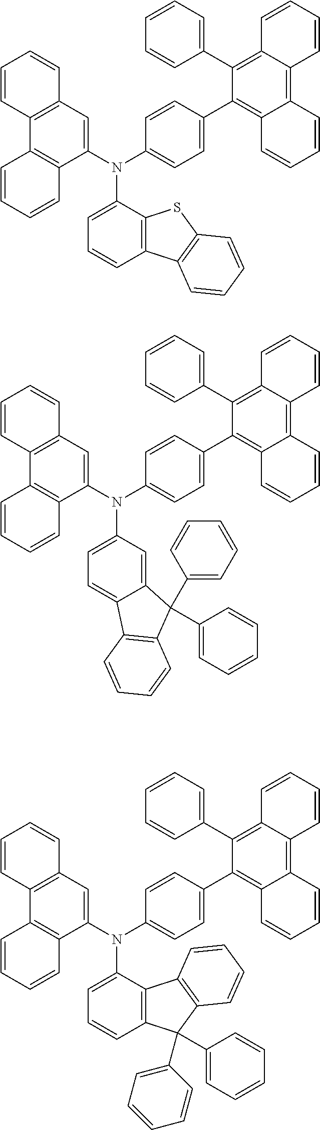

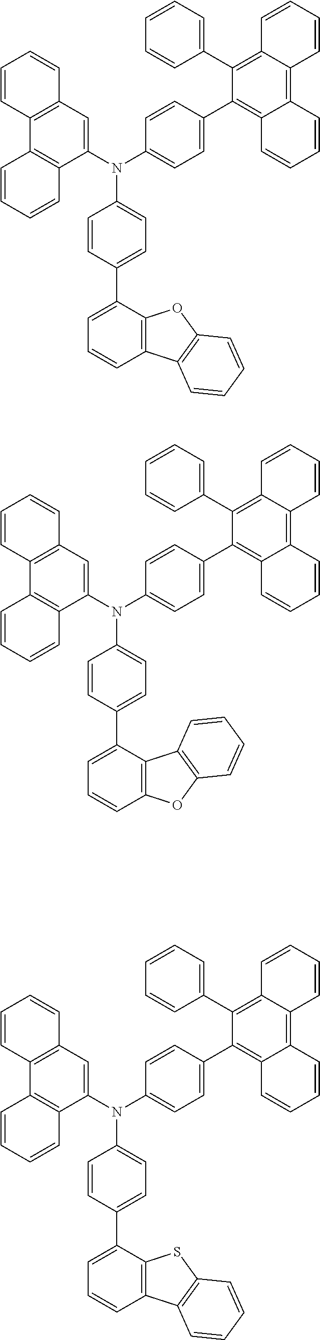 [SMILES: c1ccc(-c2c(-c3ccc(N(c4ccc(-c5cccc6c5oc5ccccc56)cc4)c4cc5ccccc5c5ccccc45)cc3)c3ccccc3c3ccccc23)cc1 ; c1ccc(-c2c(-c3ccc(N(c4ccc(-c5cccc6oc7ccccc7c56)cc4)c4cc5ccccc5c5ccccc45)cc3)c3ccccc3c3ccccc23)cc1 ; c1ccc(-c2c(-c3ccc(N(c4ccc(-c5cccc6c5sc5ccccc56)cc4)c4cc5ccccc5c5ccccc45)cc3)c3ccccc3c3ccccc23)cc1]

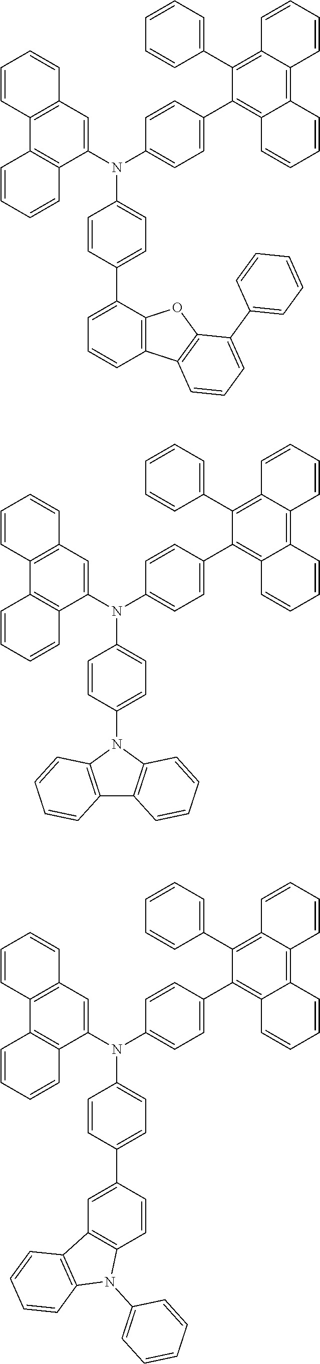 [SMILES: c1ccc(-c2c(-c3ccc(N(c4ccc(-c5cccc6c5oc5c(-c7ccccc7)cccc56)cc4)c4cc5ccccc5c5ccccc45)cc3)c3ccccc3c3ccccc23)cc1 ; c1ccc(-c2c(-c3ccc(N(c4ccc(-n5c6ccccc6c6ccccc65)cc4)c4cc5ccccc5c5ccccc45)cc3)c3ccccc3c3ccccc23)cc1 ; c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1]

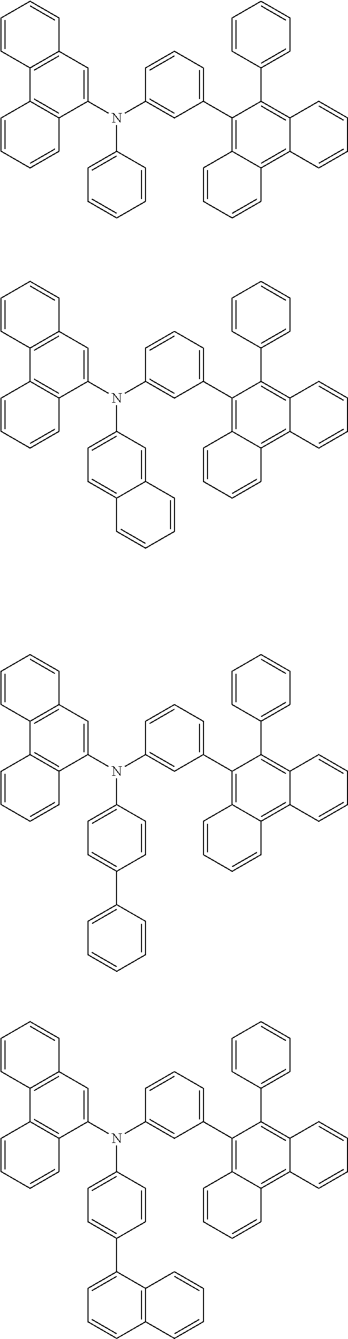 [SMILES: c1ccc(-c2c(-c3cccc(N(c4ccccc4)c4cc5ccccc5c5ccccc45)c3)c3ccccc3c3ccccc23)cc1 ; c1ccc(-c2c(-c3cccc(N(c4ccc5ccccc5c4)c4cc5ccccc5c5ccccc45)c3)c3ccccc3c3ccccc23)cc1 ; c1ccc(-c2ccc(N(c3cccc(-c4c(-c5ccccc5)c5ccccc5c5ccccc45)c3)c3cc4ccccc4c4ccccc34)cc2)cc1 ; c1ccc(-c2c(-c3cccc(N(c4ccc(-c5cccc6ccccc56)cc4)c4cc5ccccc5c5ccccc45)c3)c3ccccc3c3ccccc23)cc1]

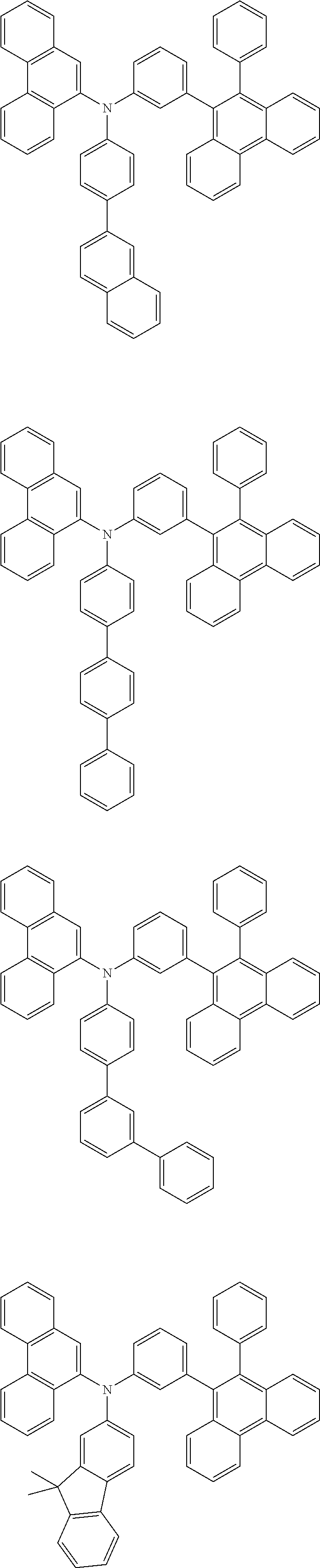 [SMILES: c1ccc(-c2c(-c3cccc(N(c4ccc(-c5ccc6ccccc6c5)cc4)c4cc5ccccc5c5ccccc45)c3)c3ccccc3c3ccccc23)cc1 ; c1ccc(-c2ccc(-c3ccc(N(c4cccc(-c5c(-c6ccccc6)c6ccccc6c6ccccc56)c4)c4cc5ccccc5c5ccccc45)cc3)cc2)cc1 ; c1ccc(-c2cccc(-c3ccc(N(c4cccc(-c5c(-c6ccccc6)c6ccccc6c6ccccc56)c4)c4cc5ccccc5c5ccccc45)cc3)c2)cc1 ; CC1(C)c2ccccc2-c2ccc(N(c3cccc(-c4c(-c5ccccc5)c5ccccc5c5ccccc45)c3)c3cc4ccccc4c4ccccc34)cc21]

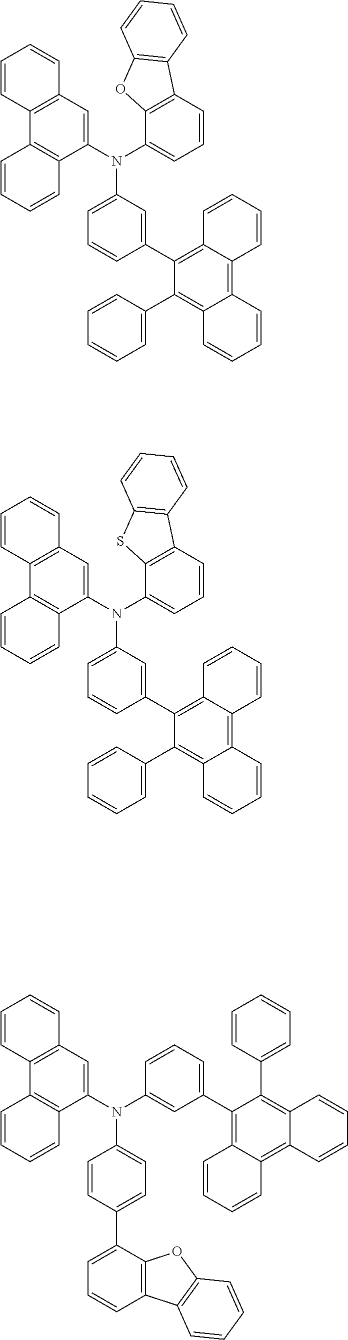 [SMILES: c1ccc(-c2c(-c3cccc(N(c4cc5ccccc5c5ccccc45)c4cccc5c4oc4ccccc45)c3)c3ccccc3c3ccccc23)cc1 ; c1ccc(-c2c(-c3cccc(N(c4cc5ccccc5c5ccccc45)c4cccc5c4sc4ccccc45)c3)c3ccccc3c3ccccc23)cc1 ; c1ccc(-c2c(-c3cccc(N(c4ccc(-c5cccc6c5oc5ccccc56)cc4)c4cc5ccccc5c5ccccc45)c3)c3ccccc3c3ccccc23)cc1]

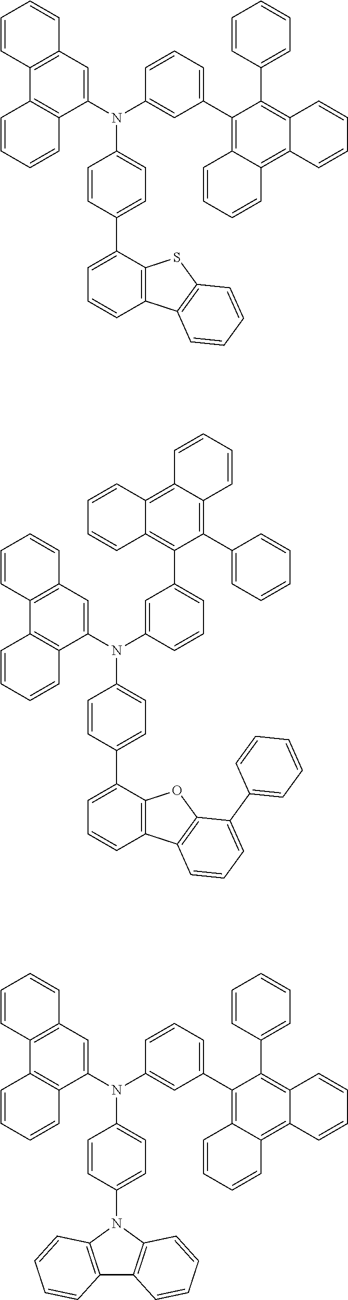 [SMILES: c1ccc(-c2c(-c3cccc(N(c4ccc(-c5cccc6c5sc5ccccc56)cc4)c4cc5ccccc5c5ccccc45)c3)c3ccccc3c3ccccc23)cc1 ; c1ccc(-c2c(-c3cccc(N(c4ccc(-c5cccc6c5oc5c(-c7ccccc7)cccc56)cc4)c4cc5ccccc5c5ccccc45)c3)c3ccccc3c3ccccc23)cc1 ; c1ccc(-c2c(-c3cccc(N(c4ccc(-n5c6ccccc6c6ccccc65)cc4)c4cc5ccccc5c5ccccc45)c3)c3ccccc3c3ccccc23)cc1]

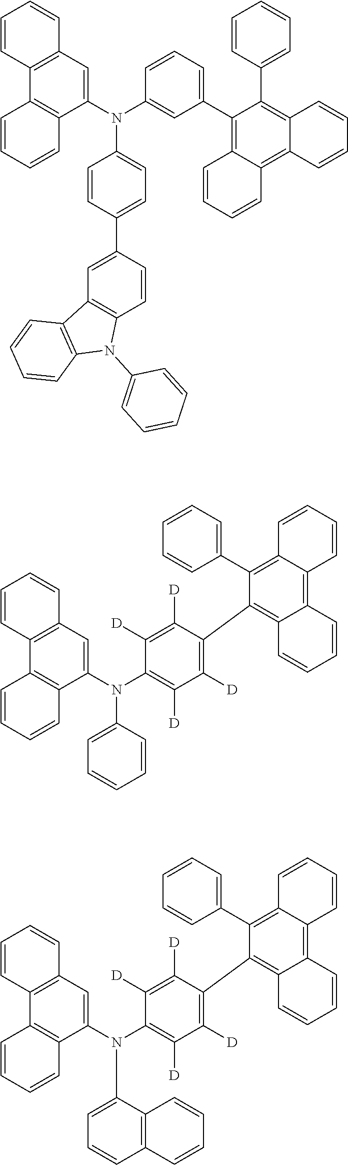 [SMILES: c1ccc(-c2c(-c3cccc(N(c4ccc(-c5ccc6c(c5)c5ccccc5n6-c5ccccc5)cc4)c4cc5ccccc5c5ccccc45)c3)c3ccccc3c3ccccc23)cc1 ; [2H]c1c([2H])c(N(c2ccccc2)c2cc3ccccc3c3ccccc23)c([2H])c([2H])c1-c1c(-c2ccccc2)c2ccccc2c2ccccc12 ; [2H]c1c([2H])c(N(c2cccc3ccccc23)c2cc3ccccc3c3ccccc23)c([2H])c([2H])c1-c1c(-c2ccccc2)c2ccccc2c2ccccc12]

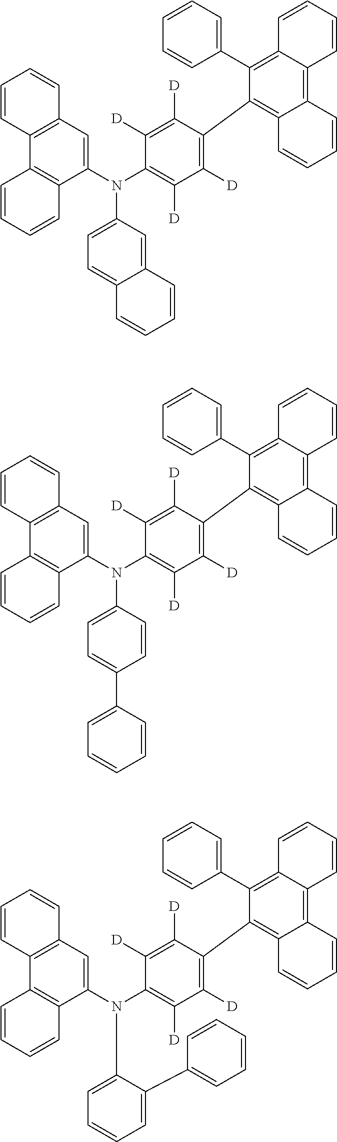 [SMILES: [2H]c1c([2H])c(N(c2ccc3ccccc3c2)c2cc3ccccc3c3ccccc23)c([2H])c([2H])c1-c1c(-c2ccccc2)c2ccccc2c2ccccc12 ; [2H]c1c([2H])c(N(c2ccc(-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccccc2-c2ccccc2)c2cc3ccccc3c3ccccc23)c([2H])c([2H])c1-c1c(-c2ccccc2)c2ccccc2c2ccccc12]

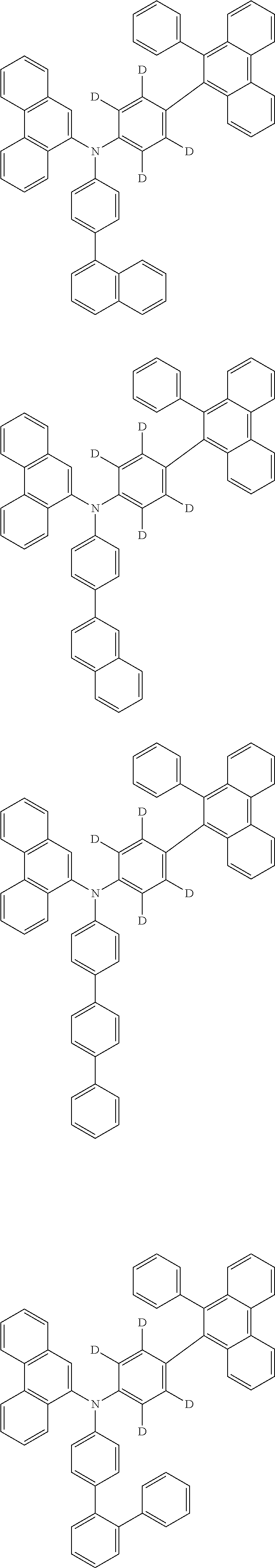 [SMILES: [2H]c1c([2H])c(N(c2ccc(-c3cccc4ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc4ccccc4c3)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccccc3-c3ccccc3)cc2)c2cc3ccccc3c3ccccc23)c([2H])c([2H])c1-c1c(-c2ccccc2)c2ccccc2c2ccccc12]

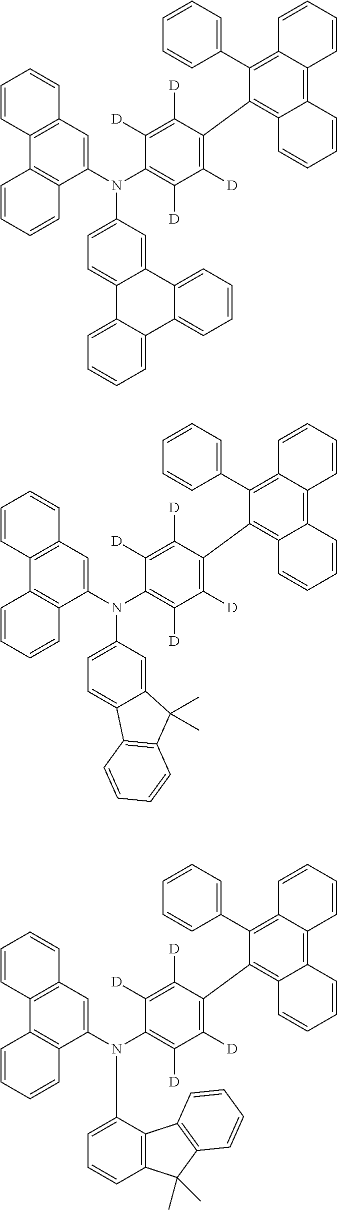 [SMILES: [2H]c1c([2H])c(N(c2ccc3c4ccccc4c4ccccc4c3c2)c2cc3ccccc3c3ccccc23)c([2H])c([2H])c1-c1c(-c2ccccc2)c2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1c(-c2ccccc2)c2ccccc2c2ccccc12 ; [2H]c1c([2H])c(N(c2cccc3c2-c2ccccc2C3(C)C)c2cc3ccccc3c3ccccc23)c([2H])c([2H])c1-c1c(-c2ccccc2)c2ccccc2c2ccccc12]

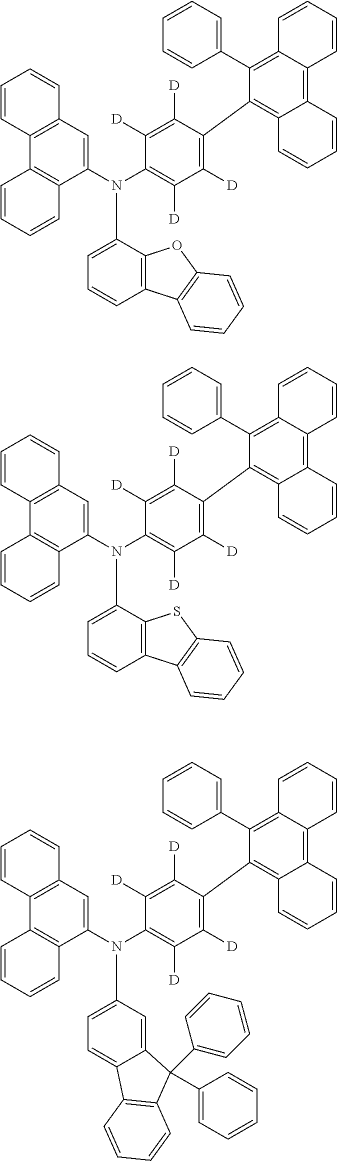

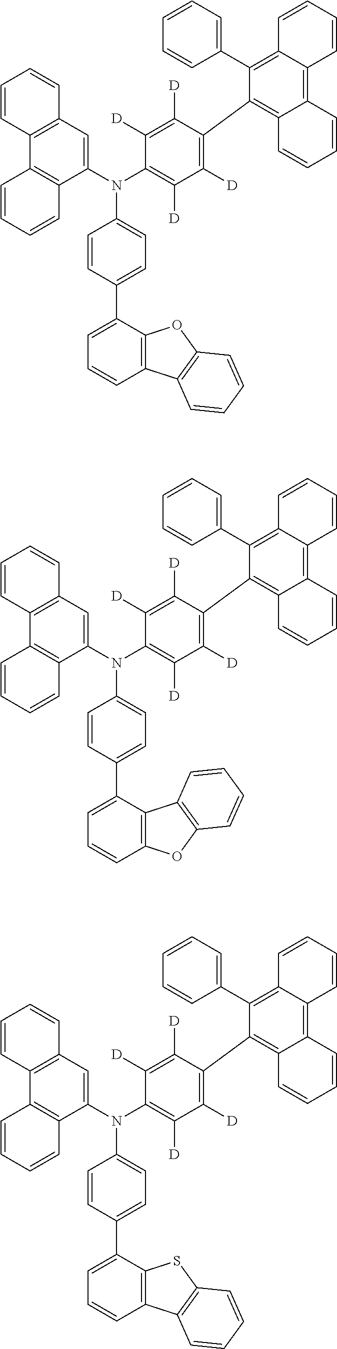 [SMILES: [2H]c1c([2H])c(N(c2ccc(-c3cccc4c3oc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3sc3ccccc34)cc2)c2cc3ccccc3c3ccccc23)c([2H])c([2H])c1-c1c(-c2ccccc2)c2ccccc2c2ccccc12]

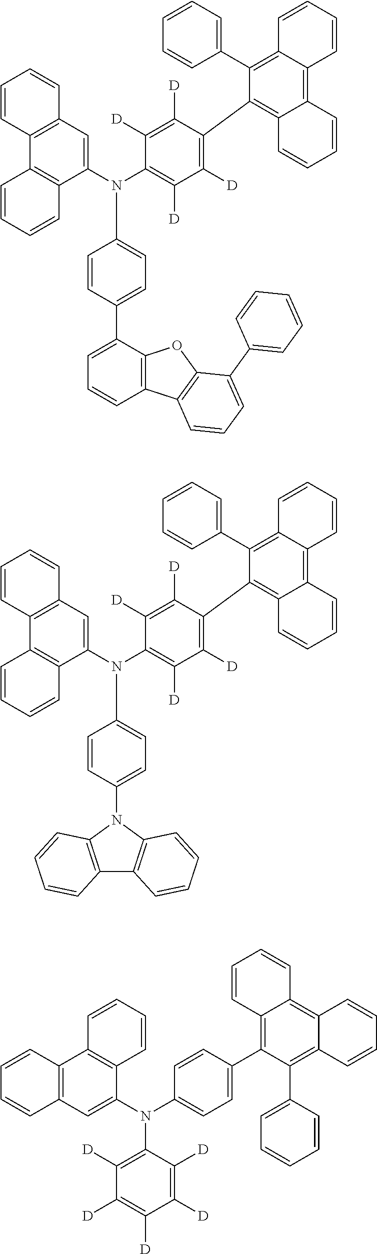 [SMILES: [2H]c1c([2H])c(N(c2ccc(-c3cccc4c3oc3c(-c5ccccc5)cccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1c(-c2ccccc2)c2ccccc2c2ccccc12 ; [2H]c1c([2H])c([2H])c(N(c2ccc(-c3c(-c4ccccc4)c4ccccc4c4ccccc34)cc2)c2cc3ccccc3c3ccccc23)c([2H])c1[2H]]

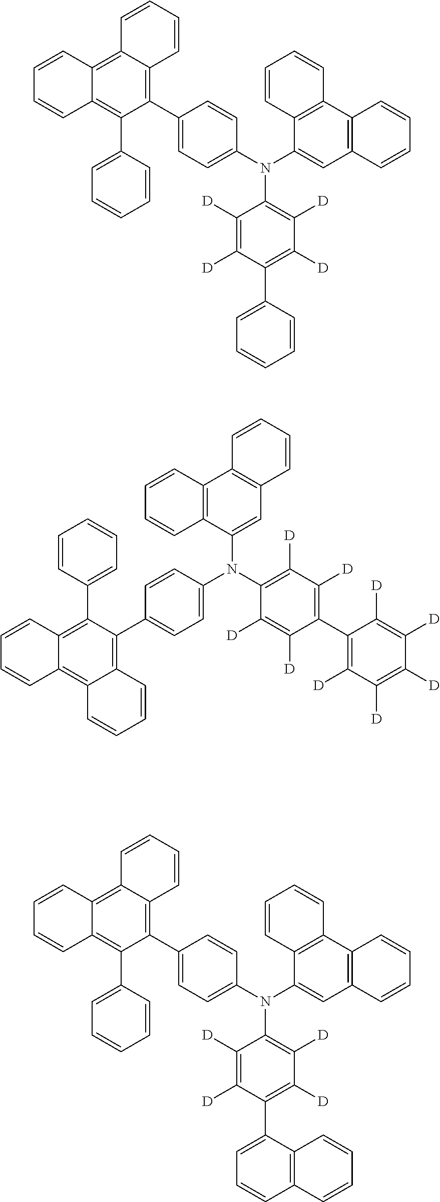 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1 ; [2H]c1c([2H])c([2H])c(-c2c([2H])c([2H])c(N(c3ccc(-c4c(-c5ccccc5)c5ccccc5c5ccccc45)cc3)c3cc4ccccc4c4ccccc34)c([2H])c2[2H])c([2H])c1[2H] ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2ccccc12]

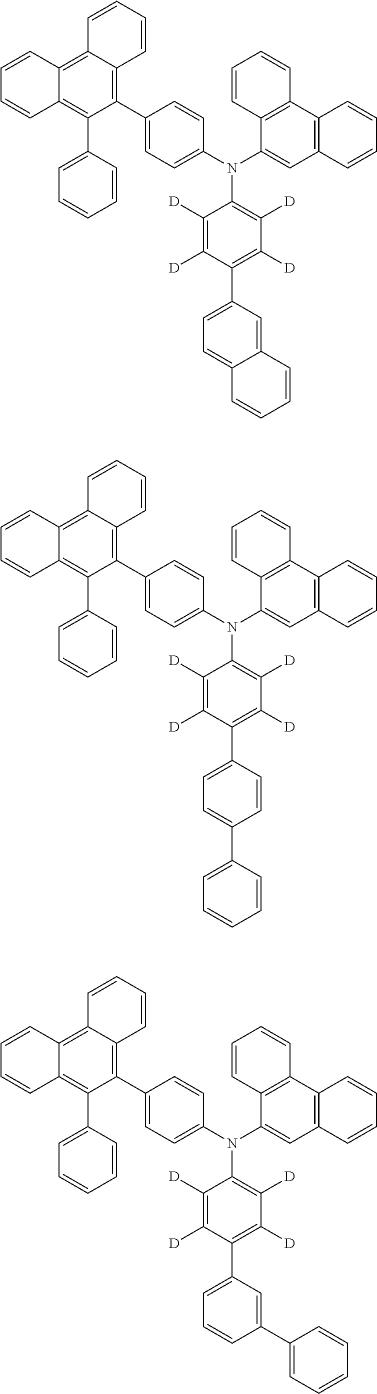 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc2ccccc2c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc(-c2ccccc2)cc1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc(-c2ccccc2)c1]

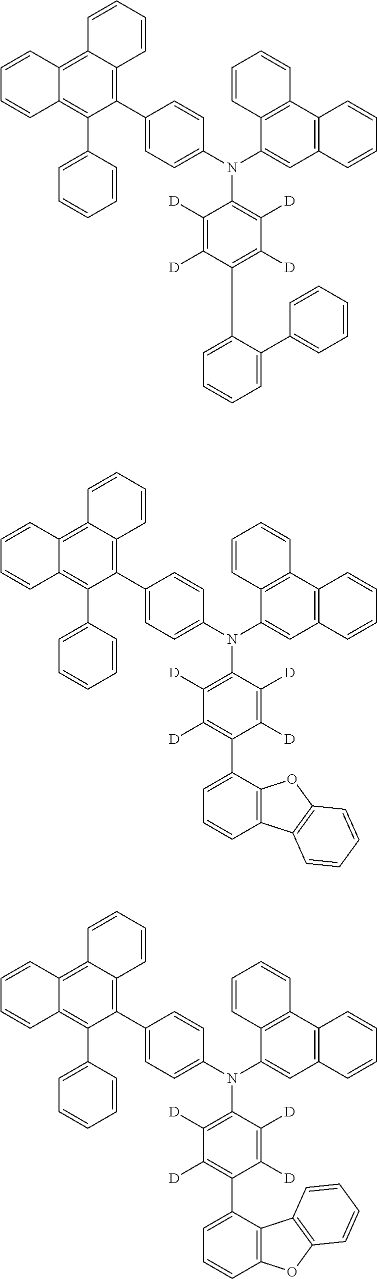 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1oc1ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2oc3ccccc3c12]

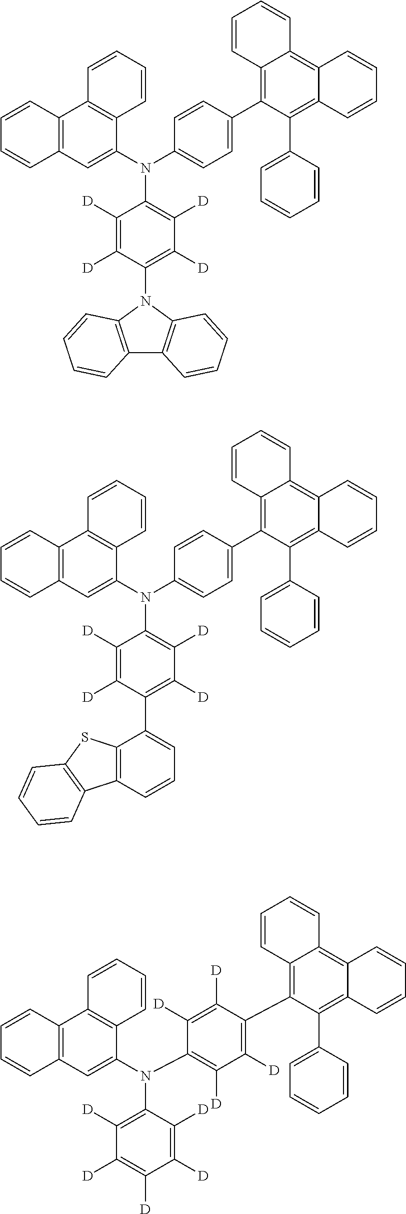 [SMILES: [2H]c1c([2H])c(-n2c3ccccc3c3ccccc32)c([2H])c([2H])c1N(c1ccc(-c2c(-c3ccccc3)c3ccccc3c3ccccc23)cc1)c1cc2ccccc2c2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1sc1ccccc12 ; [2H]c1c([2H])c([2H])c(N(c2c([2H])c([2H])c(-c3c(-c4ccccc4)c4ccccc4c4ccccc34)c([2H])c2[2H])c2cc3ccccc3c3ccccc23)c([2H])c1[2H]]

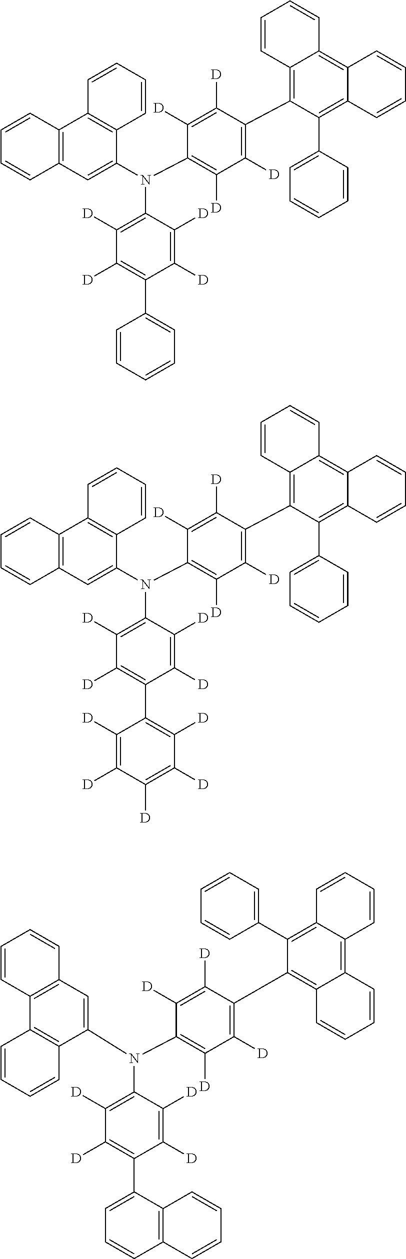 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4c(-c5ccccc5)c5ccccc5c5ccccc45)c([2H])c3[2H])c3cc4ccccc4c4ccccc34)c([2H])c2[2H])c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1cccc2ccccc12]

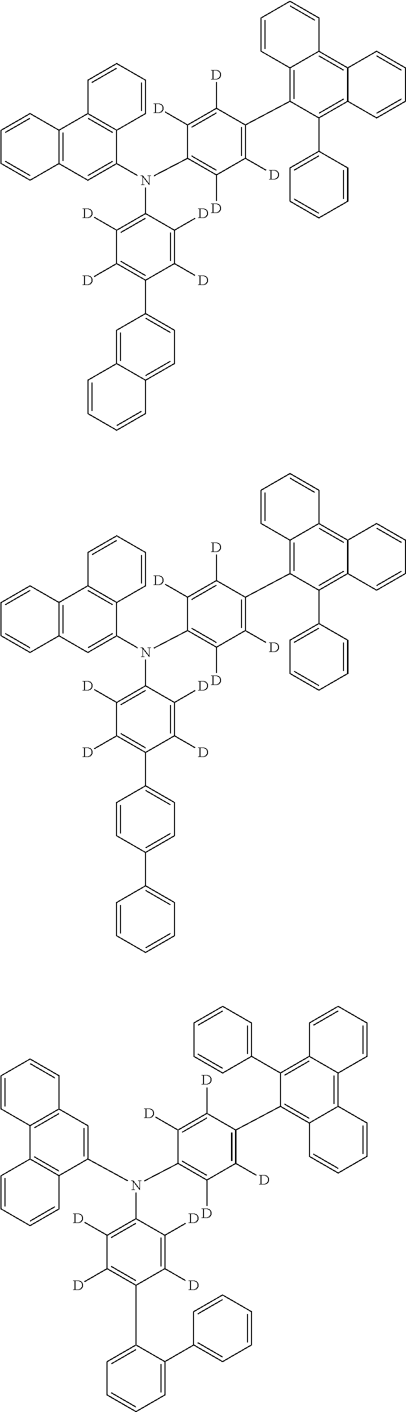 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc(-c2ccccc2)cc1 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1-c1ccccc1]

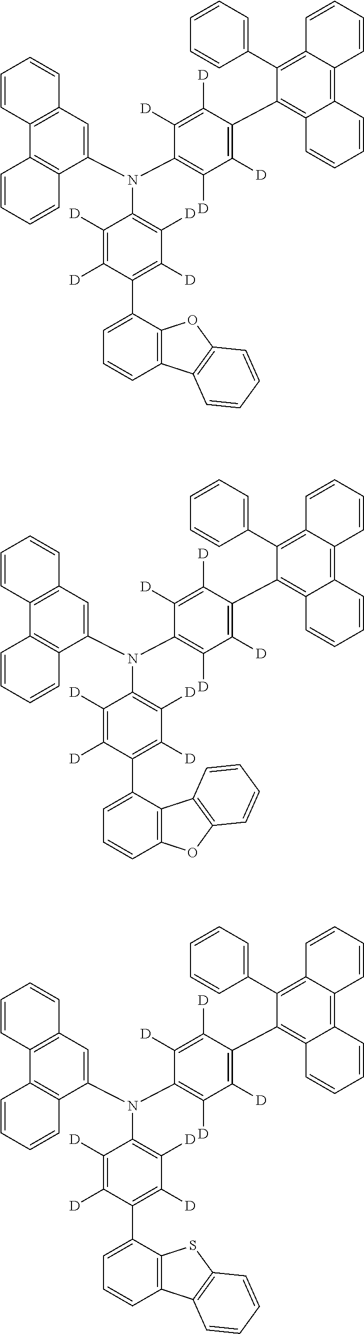 [SMILES: [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1c(-c2ccccc2)c2ccccc2c2ccccc12]

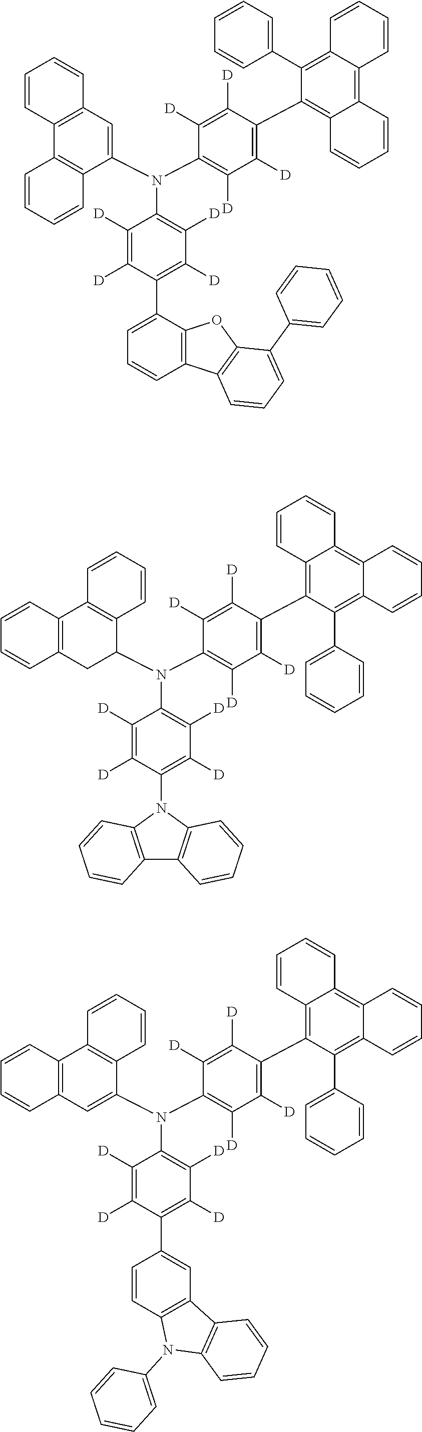 [SMILES: [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-n3c4ccccc4c4ccccc43)c([2H])c2[2H])C2Cc3ccccc3-c3ccccc32)c([2H])c([2H])c1-c1c(-c2ccccc2)c2ccccc2c2ccccc12 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2c(c1)c1ccccc1n2-c1ccccc1]

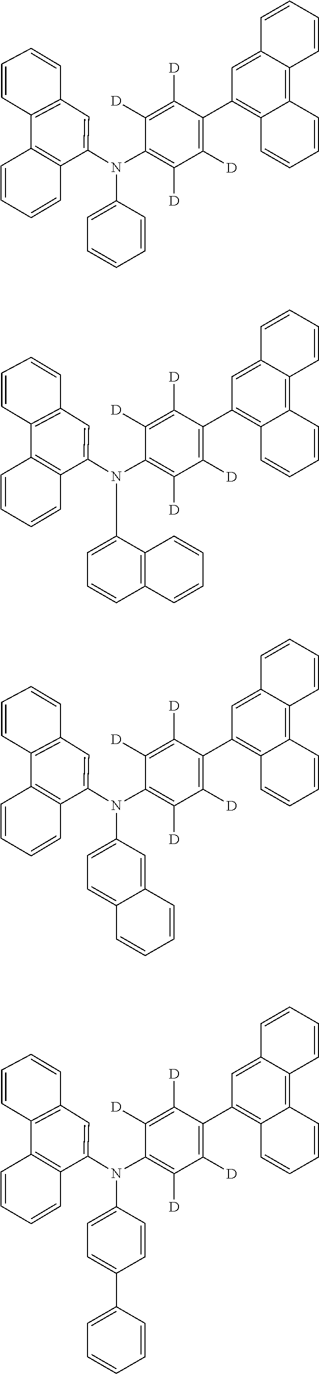 [SMILES: [2H]c1c([2H])c(N(c2ccccc2)c2cc3ccccc3c3ccccc23)c([2H])c([2H])c1-c1cc2ccccc2c2ccccc12 ; [2H]c1c([2H])c(N(c2cccc3ccccc23)c2cc3ccccc3c3ccccc23)c([2H])c([2H])c1-c1cc2ccccc2c2ccccc12 ; [2H]c1c([2H])c(N(c2ccc3ccccc3c2)c2cc3ccccc3c3ccccc23)c([2H])c([2H])c1-c1cc2ccccc2c2ccccc12 ; [2H]c1c([2H])c(N(c2ccc(-c3ccccc3)cc2)c2cc3ccccc3c3ccccc23)c([2H])c([2H])c1-c1cc2ccccc2c2ccccc12]

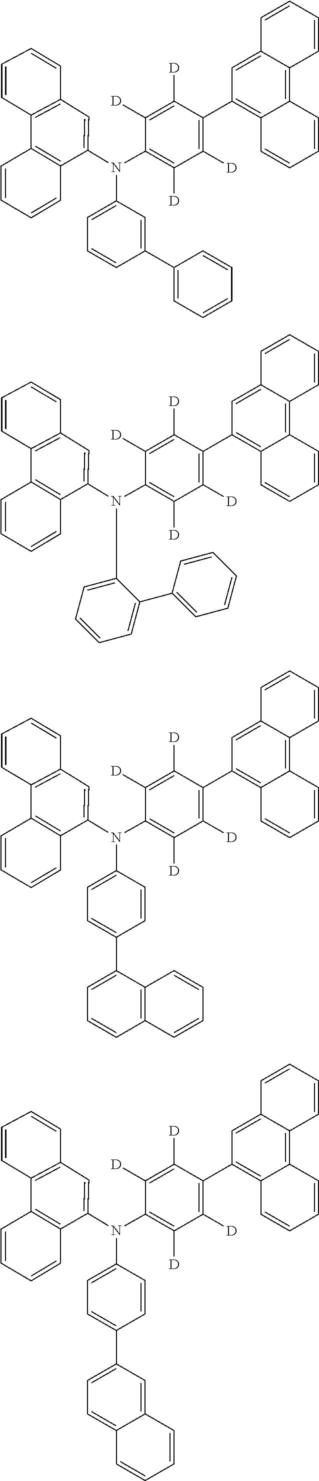 [SMILES: [2H]c1c([2H])c(N(c2cccc(-c3ccccc3)c2)c2cc3ccccc3c3ccccc23)c([2H])c([2H])c1-c1cc2ccccc2c2ccccc12 ; [2H]c1c([2H])c(N(c2ccccc2-c2ccccc2)c2cc3ccccc3c3ccccc23)c([2H])c([2H])c1-c1cc2ccccc2c2ccccc12 ; [2H]c1c([2H])c(N(c2ccc(-c3cccc4ccccc34)cc2)c2cc3ccccc3c3ccccc23)c([2H])c([2H])c1-c1cc2ccccc2c2ccccc12 ; [2H]c1c([2H])c(N(c2ccc(-c3ccc4ccccc4c3)cc2)c2cc3ccccc3c3ccccc23)c([2H])c([2H])c1-c1cc2ccccc2c2ccccc12]

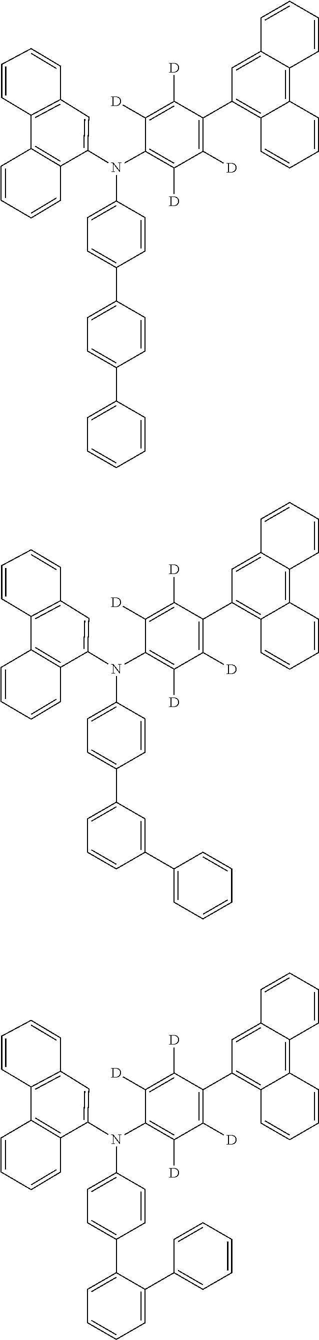 [SMILES: [2H]c1c([2H])c(N(c2ccc(-c3ccc(-c4ccccc4)cc3)cc2)c2cc3ccccc3c3ccccc23)c([2H])c([2H])c1-c1cc2ccccc2c2ccccc12 ; [2H]c1c([2H])c(N(c2ccc(-c3cccc(-c4ccccc4)c3)cc2)c2cc3ccccc3c3ccccc23)c([2H])c([2H])c1-c1cc2ccccc2c2ccccc12 ; [2H]c1c([2H])c(N(c2ccc(-c3ccccc3-c3ccccc3)cc2)c2cc3ccccc3c3ccccc23)c([2H])c([2H])c1-c1cc2ccccc2c2ccccc12]

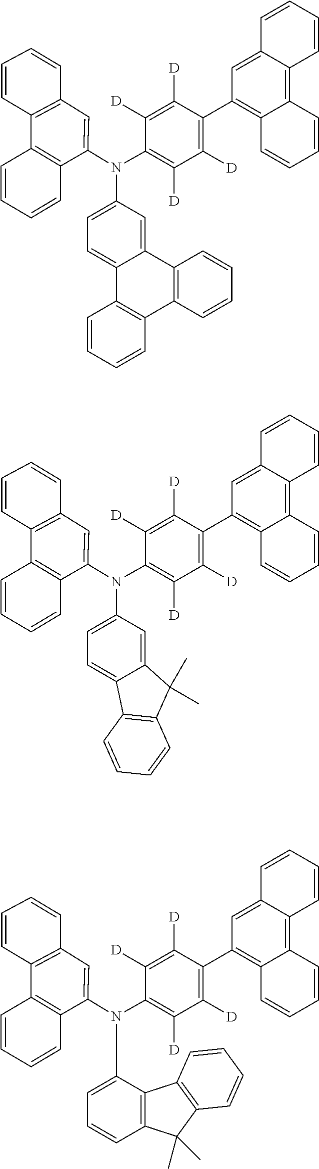 [SMILES: [2H]c1c([2H])c(N(c2ccc3c4ccccc4c4ccccc4c3c2)c2cc3ccccc3c3ccccc23)c([2H])c([2H])c1-c1cc2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(C)C)c2cc3ccccc3c3ccccc23)c([2H])c([2H])c1-c1cc2ccccc2c2ccccc12]

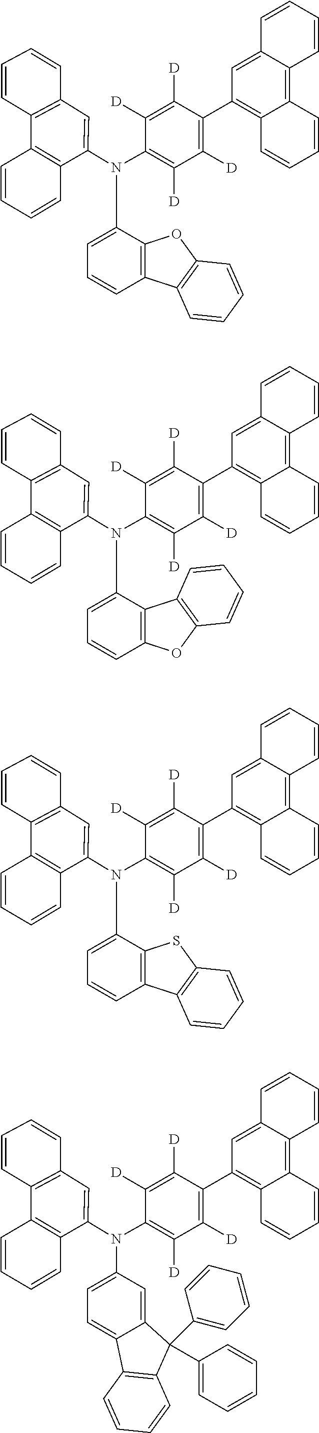 [SMILES: [2H]c1c([2H])c(N(c2cc3ccccc3c3ccccc23)c2cccc3c2oc2ccccc23)c([2H])c([2H])c1-c1cc2ccccc2c2ccccc12 ; [2H]c1c([2H])c(N(c2cc3ccccc3c3ccccc23)c2cccc3oc4ccccc4c23)c([2H])c([2H])c1-c1cc2ccccc2c2ccccc12 ; [2H]c1c([2H])c(N(c2cc3ccccc3c3ccccc23)c2cccc3c2sc2ccccc23)c([2H])c([2H])c1-c1cc2ccccc2c2ccccc12 ; [2H]c1c([2H])c(N(c2ccc3c(c2)C(c2ccccc2)(c2ccccc2)c2ccccc2-3)c2cc3ccccc3c3ccccc23)c([2H])c([2H])c1-c1cc2ccccc2c2ccccc12]

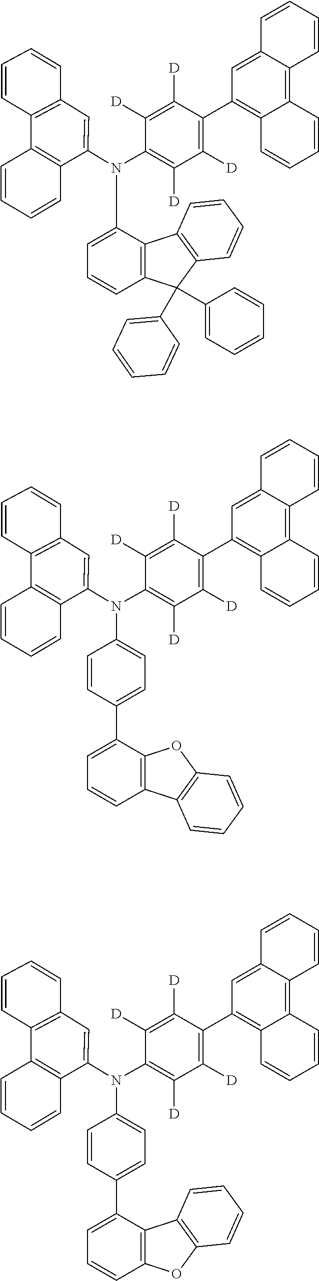 [SMILES: [2H]c1c([2H])c(N(c2cccc3c2-c2ccccc2C3(c2ccccc2)c2ccccc2)c2cc3ccccc3c3ccccc23)c([2H])c([2H])c1-c1cc2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1cc2ccccc2c2ccccc12]

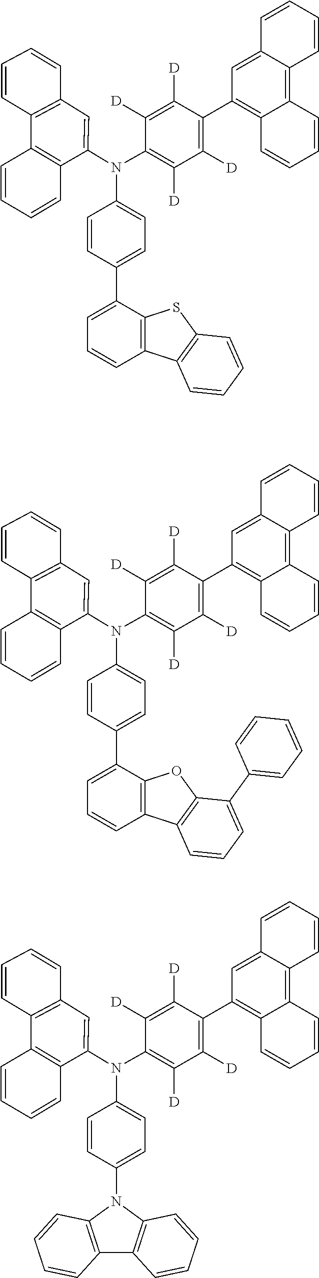 [SMILES: [2H]c1c([2H])c(N(c2ccc(-c3cccc4c3sc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1cc2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1cc2ccccc2c2ccccc12]

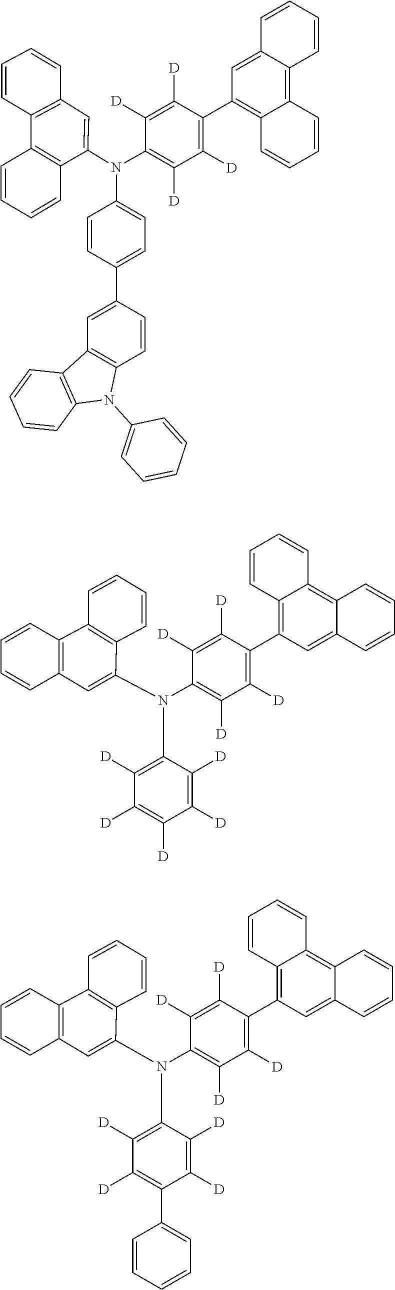 [SMILES: [2H]c1c([2H])c(N(c2ccc(-c3ccc4c(c3)c3ccccc3n4-c3ccccc3)cc2)c2cc3ccccc3c3ccccc23)c([2H])c([2H])c1-c1cc2ccccc2c2ccccc12 ; [2H]c1c([2H])c([2H])c(N(c2c([2H])c([2H])c(-c3cc4ccccc4c4ccccc34)c([2H])c2[2H])c2cc3ccccc3c3ccccc23)c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1]

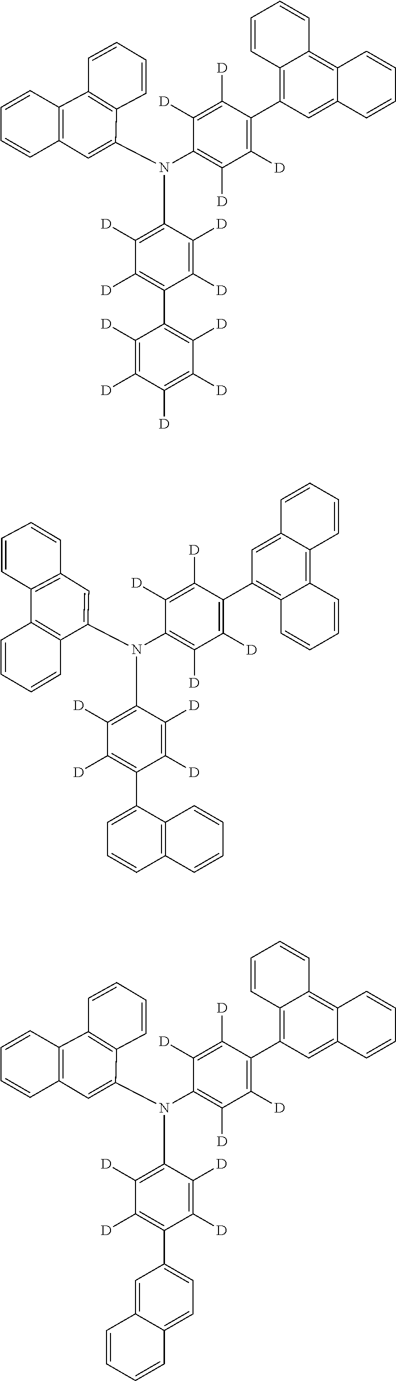 [SMILES: [2H]c1c([2H])c([2H])c(-c2c([2H])c([2H])c(N(c3c([2H])c([2H])c(-c4cc5ccccc5c5ccccc45)c([2H])c3[2H])c3cc4ccccc4c4ccccc34)c([2H])c2[2H])c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2ccccc2c1]

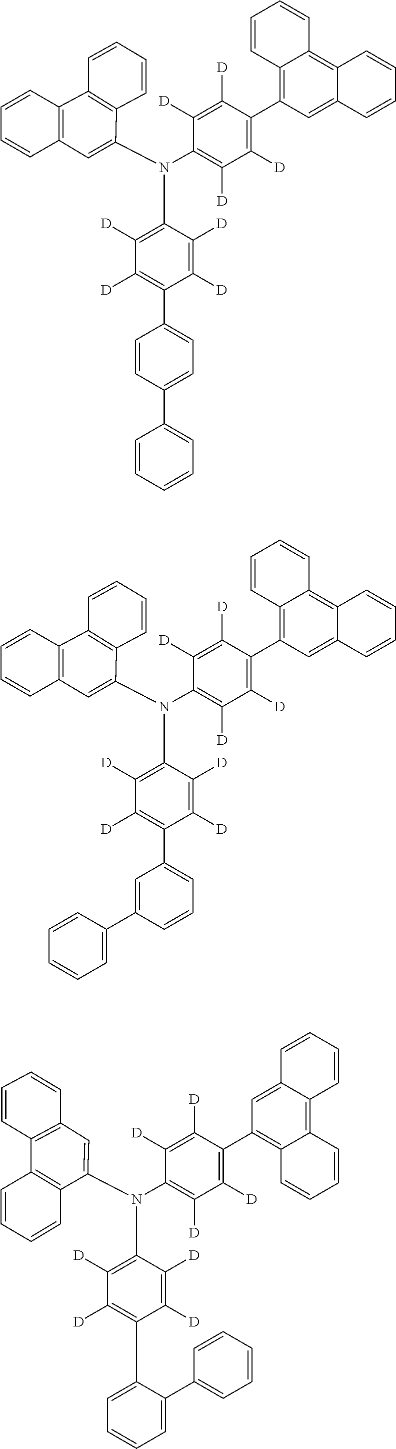 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc(-c2ccccc2)cc1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc(-c2ccccc2)c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1-c1ccccc1]

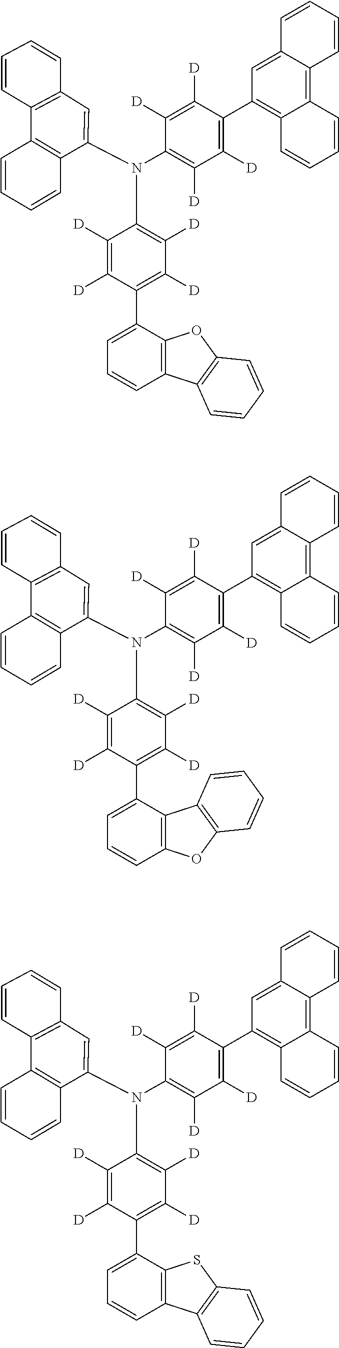 [SMILES: [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1cc2ccccc2c2ccccc12]

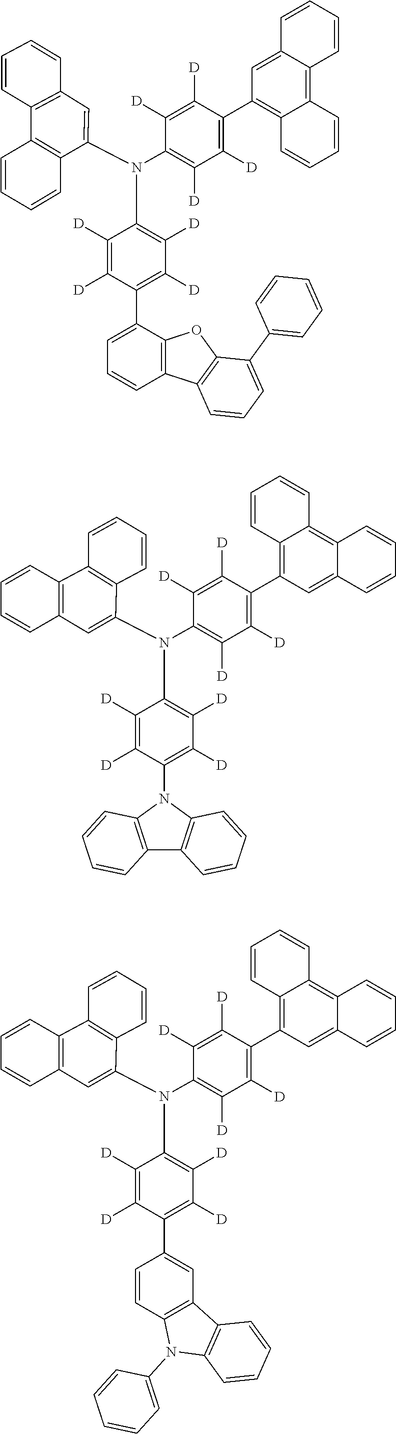 [SMILES: [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2c(c1)c1ccccc1n2-c1ccccc1]

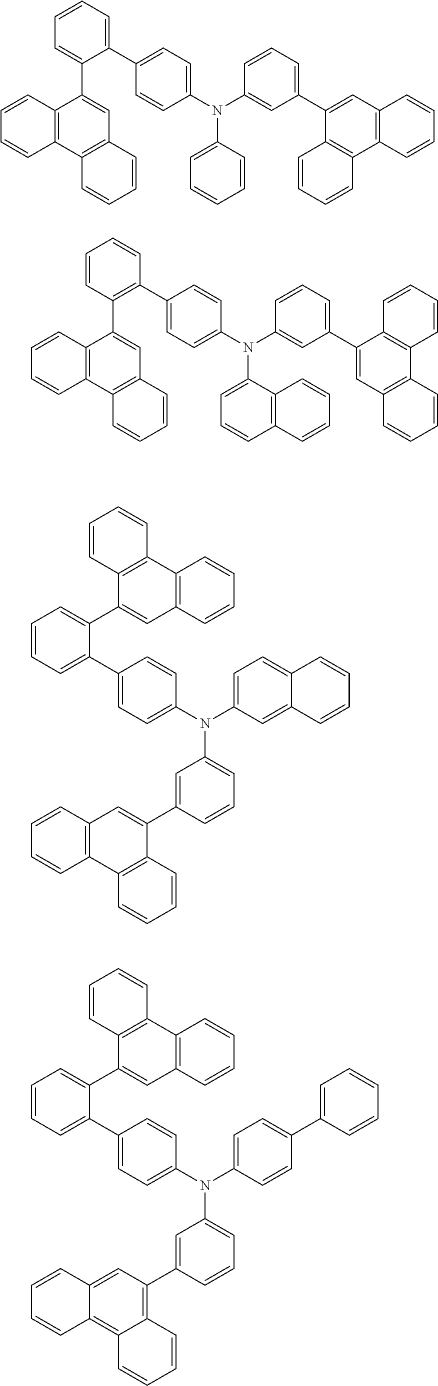

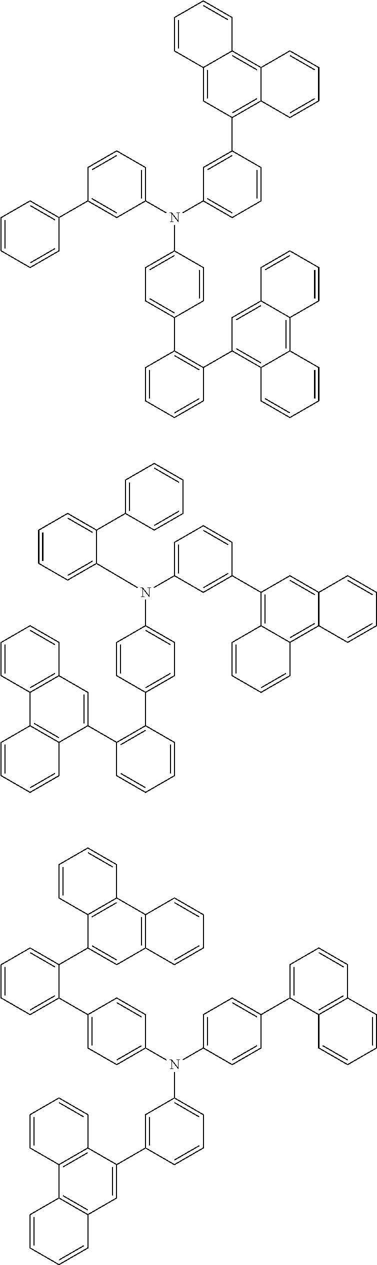 [SMILES: c1ccc(-c2cccc(N(c3ccc(-c4ccccc4-c4cc5ccccc5c5ccccc45)cc3)c3cccc(-c4cc5ccccc5c5ccccc45)c3)c2)cc1 ; c1ccc(-c2ccccc2N(c2ccc(-c3ccccc3-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3ccccc3-c3cc4ccccc4c4ccccc34)cc2)c2ccc(-c3cccc4ccccc34)cc2)c1]

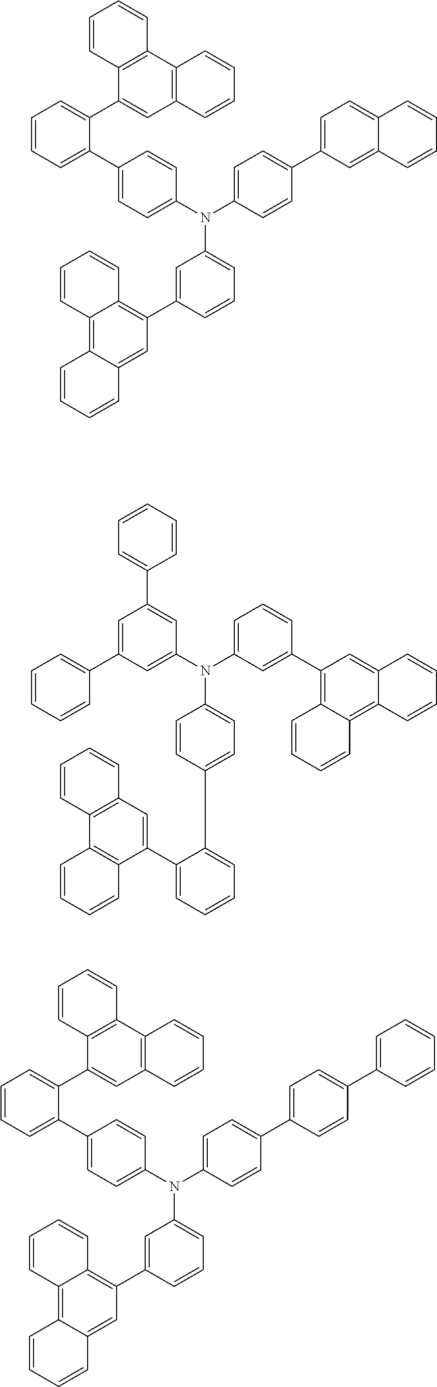 [SMILES: c1cc(-c2cc3ccccc3c3ccccc23)cc(N(c2ccc(-c3ccc4ccccc4c3)cc2)c2ccc(-c3ccccc3-c3cc4ccccc4c4ccccc34)cc2)c1 ; c1ccc(-c2cc(-c3ccccc3)cc(N(c3ccc(-c4ccccc4-c4cc5ccccc5c5ccccc45)cc3)c3cccc(-c4cc5ccccc5c5ccccc45)c3)c2)cc1 ; c1ccc(-c2ccc(-c3ccc(N(c4ccc(-c5ccccc5-c5cc6ccccc6c6ccccc56)cc4)c4cccc(-c5cc6ccccc6c6ccccc56)c4)cc3)cc2)cc1]

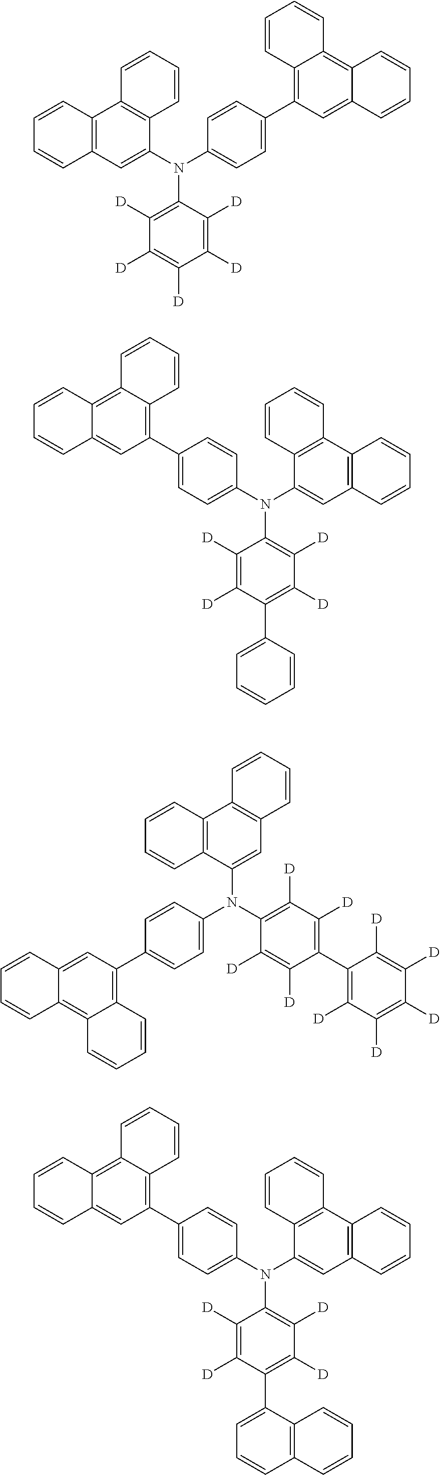

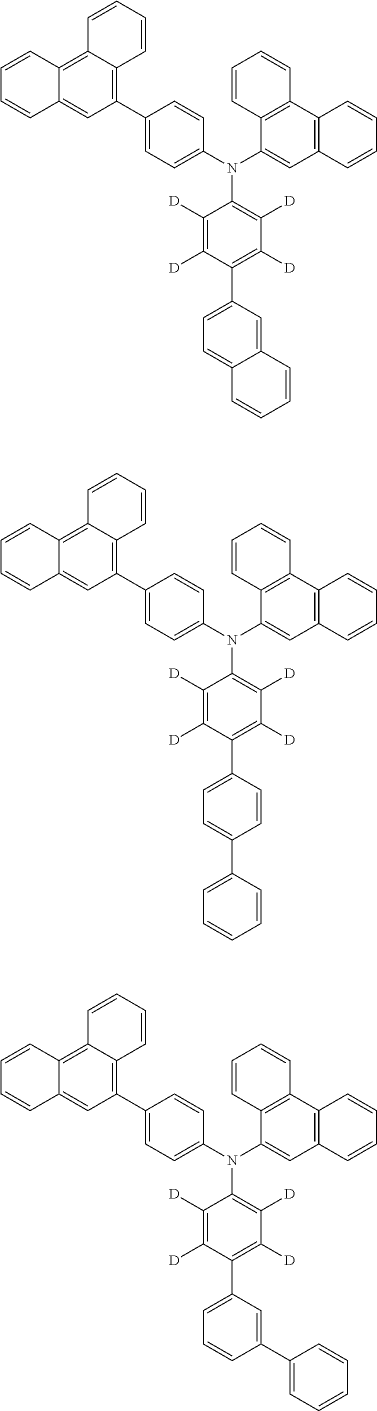 [SMILES: [2H]c1c([2H])c(N(c2ccc(-c3cc4ccccc4c4ccccc34)cc2)c2cc3ccccc3c3ccccc23)c([2H])c([2H])c1-c1ccc2ccccc2c1 ; [2H]c1c([2H])c(N(c2ccc(-c3cc4ccccc4c4ccccc34)cc2)c2cc3ccccc3c3ccccc23)c([2H])c([2H])c1-c1ccc(-c2ccccc2)cc1 ; [2H]c1c([2H])c(N(c2ccc(-c3cc4ccccc4c4ccccc34)cc2)c2cc3ccccc3c3ccccc23)c([2H])c([2H])c1-c1cccc(-c2ccccc2)c1]

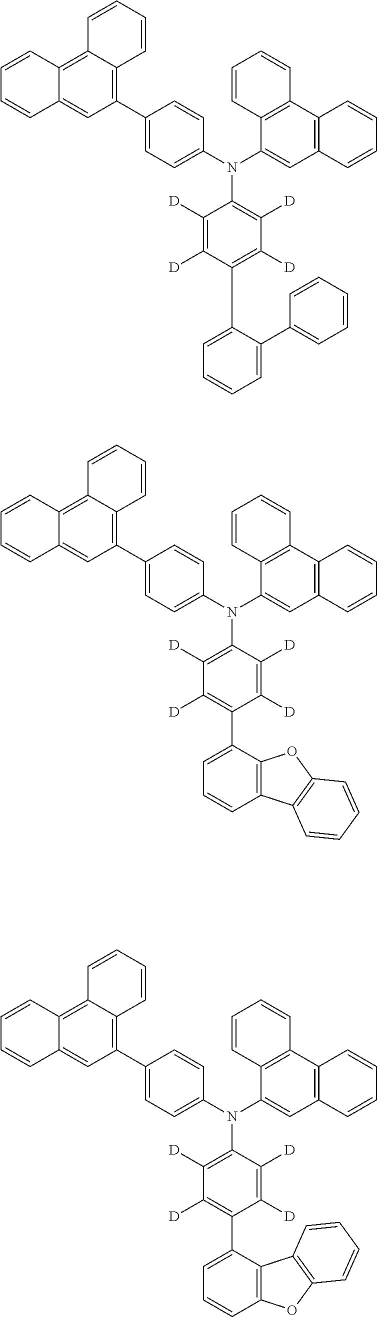 [SMILES: [2H]c1c([2H])c(N(c2ccc(-c3cc4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3cc4ccccc4c4ccccc34)cc2)c2cc3ccccc3c3ccccc23)c([2H])c([2H])c1-c1cccc2c1oc1ccccc12 ; [2H]c1c([2H])c(N(c2ccc(-c3cc4ccccc4c4ccccc34)cc2)c2cc3ccccc3c3ccccc23)c([2H])c([2H])c1-c1cccc2oc3ccccc3c12]

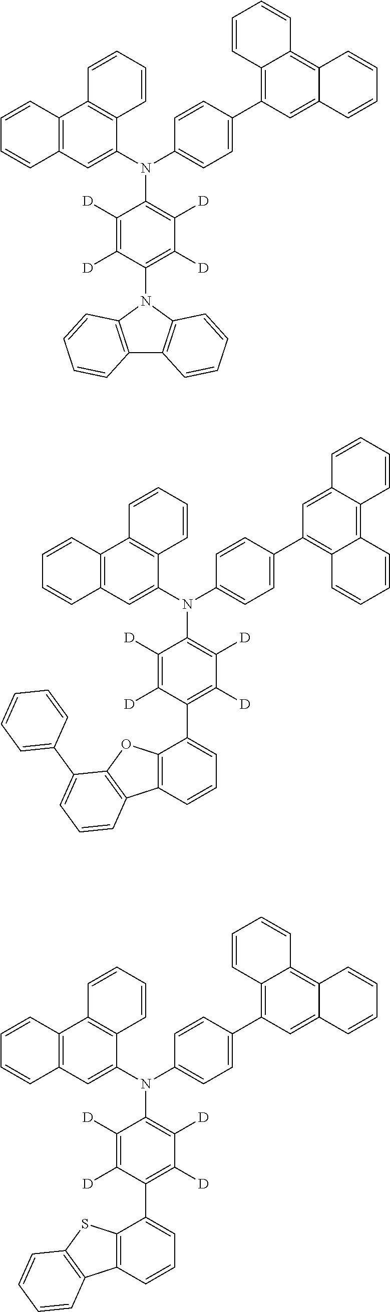 [SMILES: [2H]c1c([2H])c(-n2c3ccccc3c3ccccc32)c([2H])c([2H])c1N(c1ccc(-c2cc3ccccc3c3ccccc23)cc1)c1cc2ccccc2c2ccccc12 ; [2H]c1c([2H])c(N(c2ccc(-c3cc4ccccc4c4ccccc34)cc2)c2cc3ccccc3c3ccccc23)c([2H])c([2H])c1-c1cccc2c1oc1c(-c3ccccc3)cccc12 ; [2H]c1c([2H])c(N(c2ccc(-c3cc4ccccc4c4ccccc34)cc2)c2cc3ccccc3c3ccccc23)c([2H])c([2H])c1-c1cccc2c1sc1ccccc12]

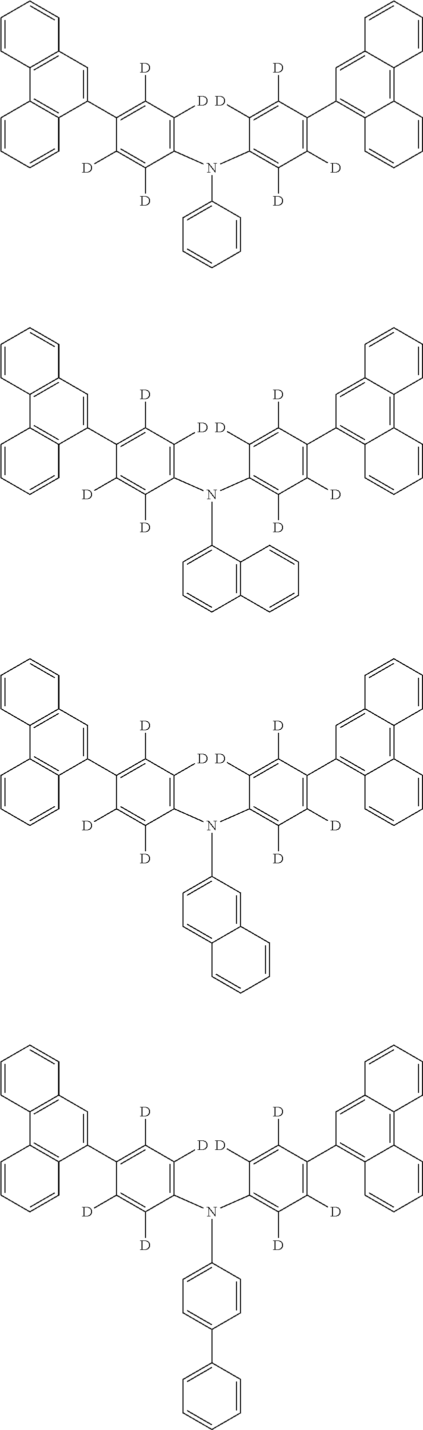 [SMILES: [2H]c1c([2H])c(N(c2ccccc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cccc3ccccc23)c([2H])c([2H])c1-c1cc2ccccc2c2ccccc12 ; [2H]c1c([2H])c(N(c2ccc3ccccc3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccccc3)cc2)c2c([2H])c([2H])c(-c3cc4ccccc4c4ccccc34)c([2H])c2[2H])c([2H])c([2H])c1-c1cc2ccccc2c2ccccc12]

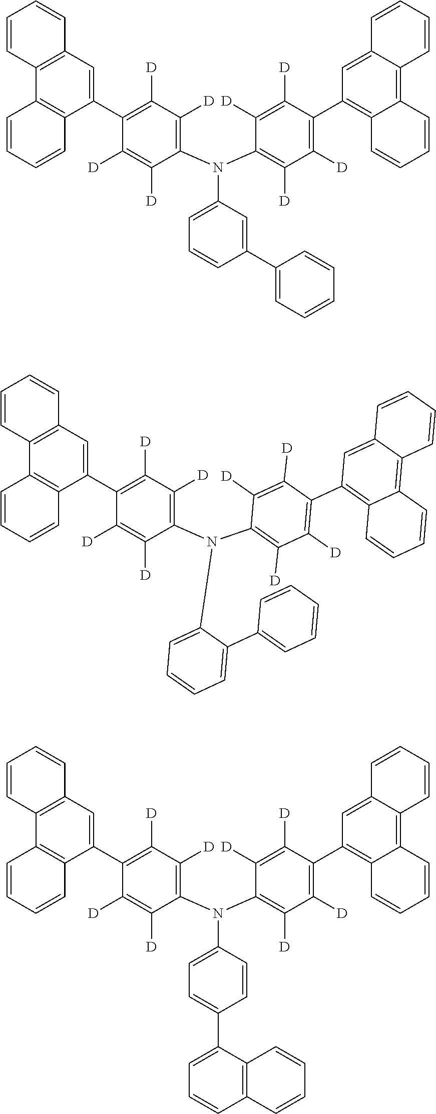 [SMILES: [2H]c1c([2H])c(N(c2cccc(-c3ccccc3)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccccc2-c2ccccc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc4ccccc34)cc2)c2c([2H])c([2H])c(-c3cc4ccccc4c4ccccc34)c([2H])c2[2H])c([2H])c([2H])c1-c1cc2ccccc2c2ccccc12]

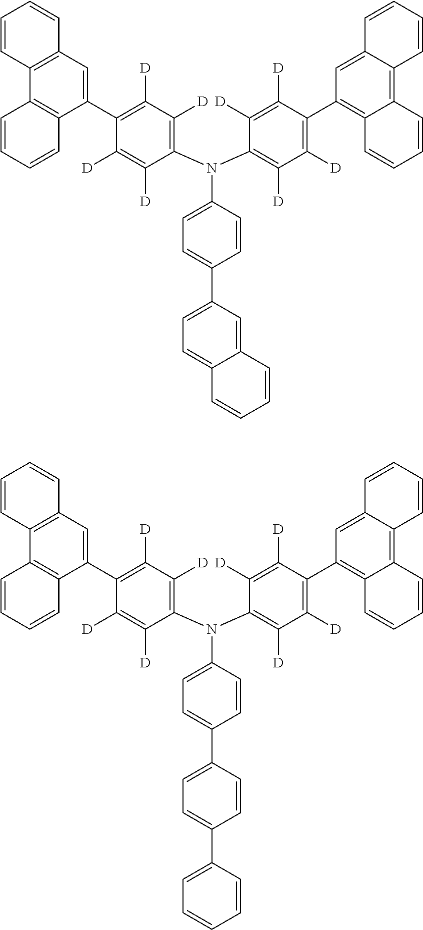 [SMILES: [2H]c1c([2H])c(N(c2ccc(-c3ccc4ccccc4c3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccc(-c4ccccc4)cc3)cc2)c2c([2H])c([2H])c(-c3cc4ccccc4c4ccccc34)c([2H])c2[2H])c([2H])c([2H])c1-c1cc2ccccc2c2ccccc12]

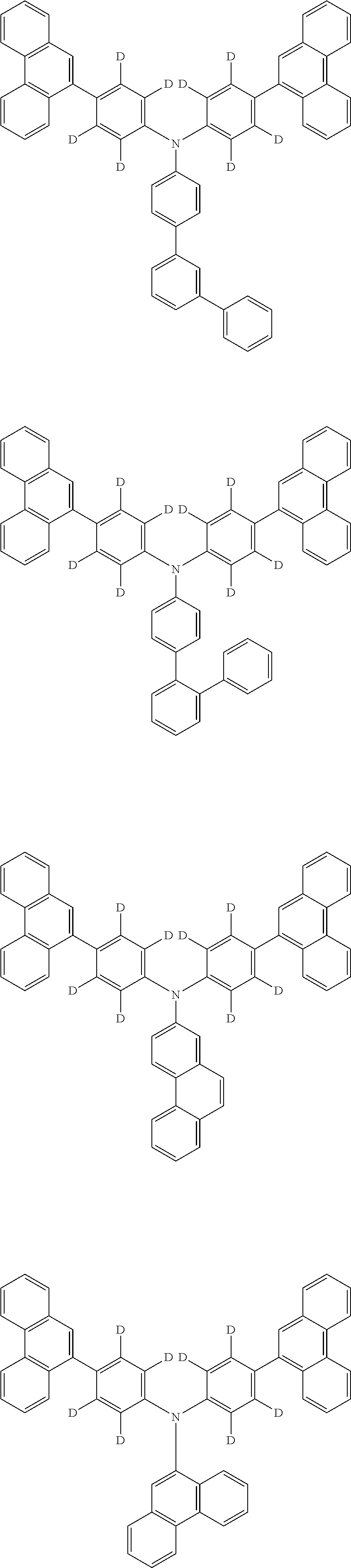

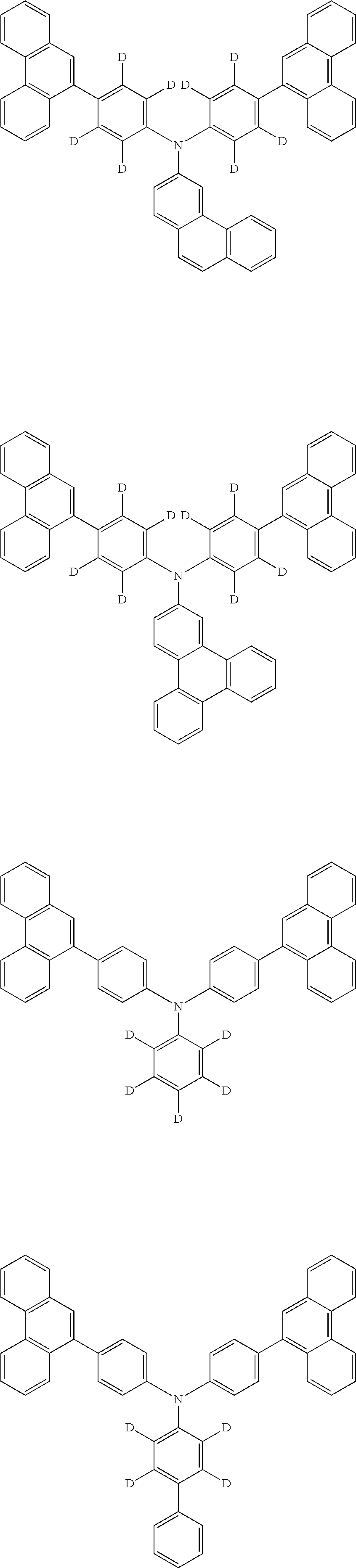 [SMILES: [2H]c1c([2H])c(N(c2ccc3ccc4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c4ccccc4c4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c([2H])c(N(c2ccc(-c3cc4ccccc4c4ccccc34)cc2)c2ccc(-c3cc4ccccc4c4ccccc34)cc2)c([2H])c1[2H] ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1]

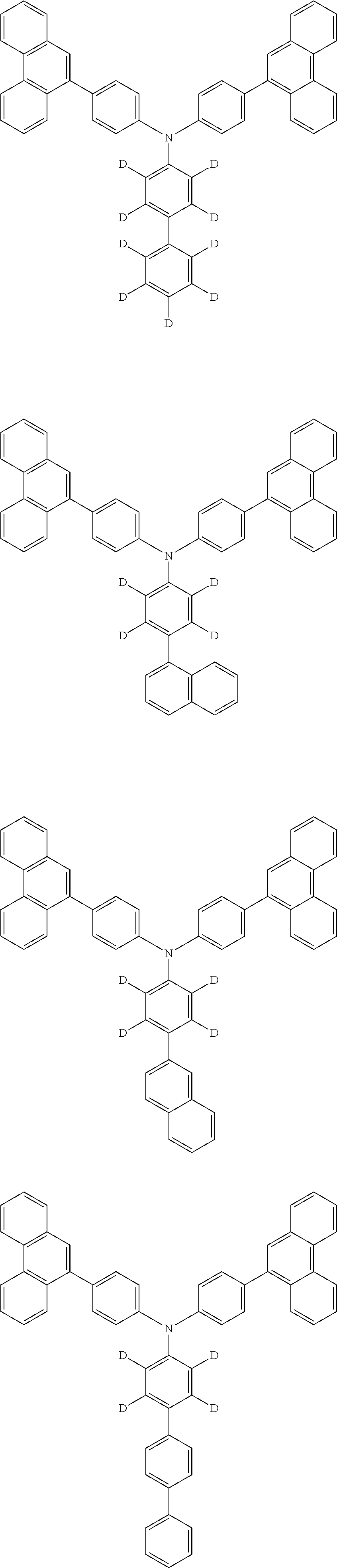 [SMILES: [2H]c1c([2H])c([2H])c(-c2c([2H])c([2H])c(N(c3ccc(-c4cc5ccccc5c5ccccc45)cc3)c3ccc(-c4cc5ccccc5c5ccccc45)cc3)c([2H])c2[2H])c([2H])c1[2H] ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1cccc2ccccc12 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc2ccccc2c1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc(-c2ccccc2)cc1]

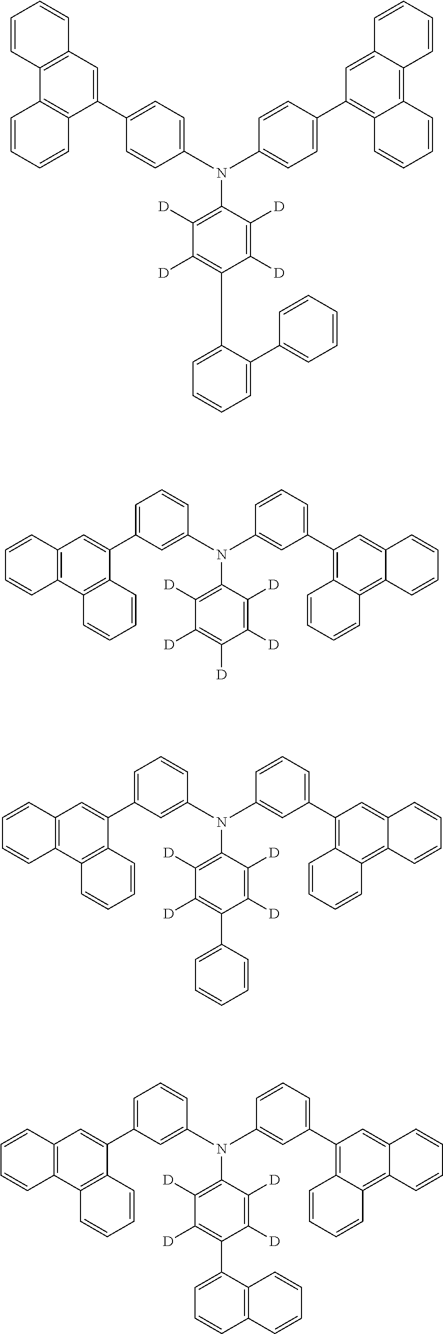 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1-c1ccccc1 ; [2H]c1c([2H])c([2H])c(N(c2cccc(-c3cc4ccccc4c4ccccc34)c2)c2cccc(-c3cc4ccccc4c4ccccc34)c2)c([2H])c1[2H] ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccccc1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1cccc2ccccc12]

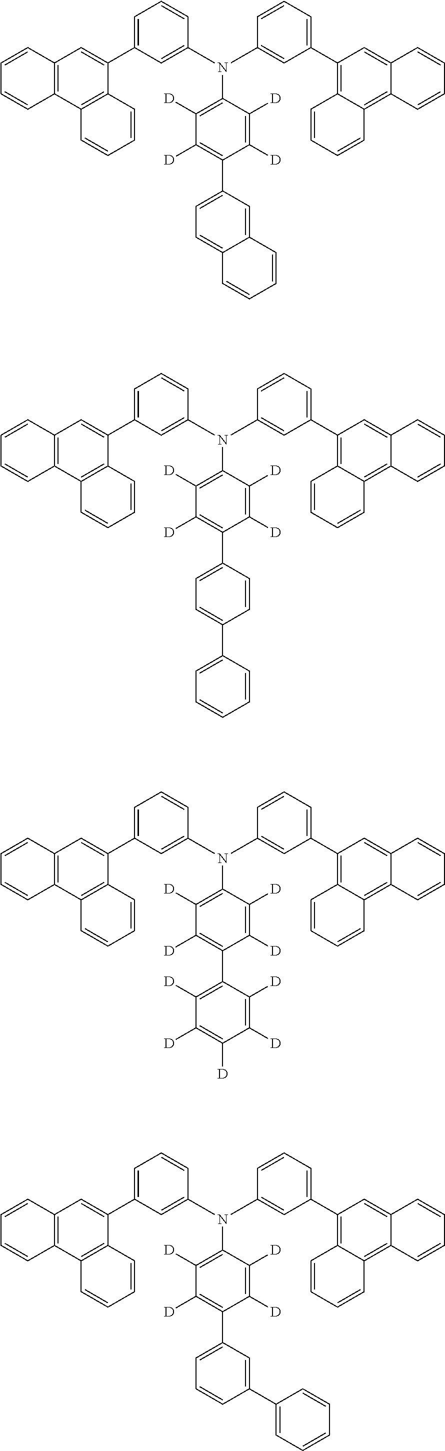 [SMILES: [2H]c1c([2H])c(N(c2cccc(-c3cc4ccccc4c4ccccc34)c2)c2cccc(-c3cc4ccccc4c4ccccc34)c2)c([2H])c([2H])c1-c1ccc2ccccc2c1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccc(-c2ccccc2)cc1 ; [2H]c1c([2H])c([2H])c(-c2c([2H])c([2H])c(N(c3cccc(-c4cc5ccccc5c5ccccc45)c3)c3cccc(-c4cc5ccccc5c5ccccc45)c3)c([2H])c2[2H])c([2H])c1[2H] ; [2H]c1c([2H])c(N(c2cccc(-c3cc4ccccc4c4ccccc34)c2)c2cccc(-c3cc4ccccc4c4ccccc34)c2)c([2H])c([2H])c1-c1cccc(-c2ccccc2)c1]

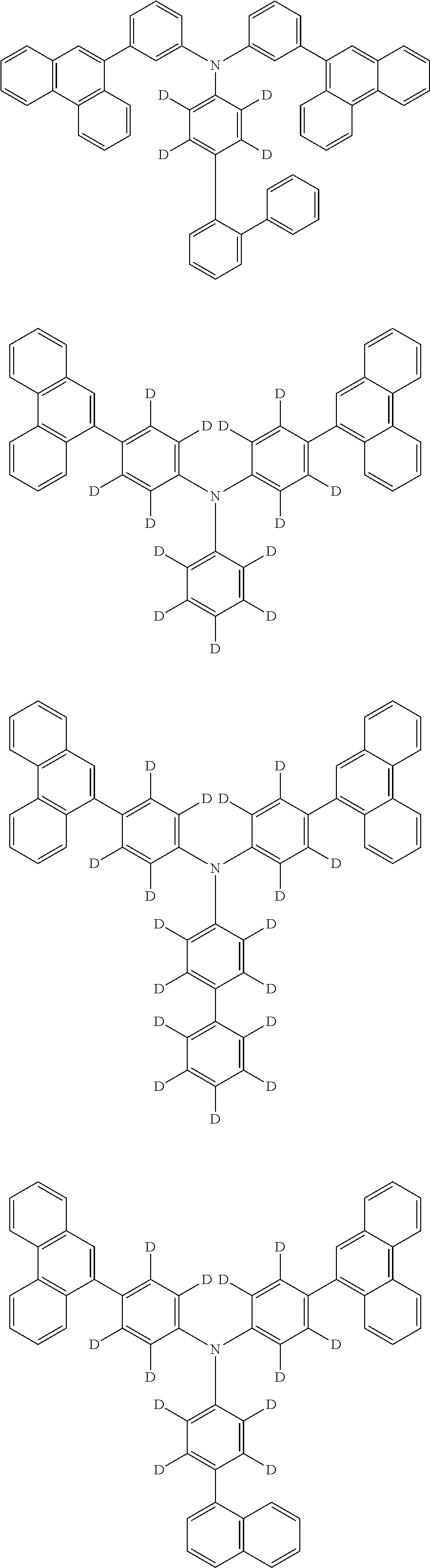

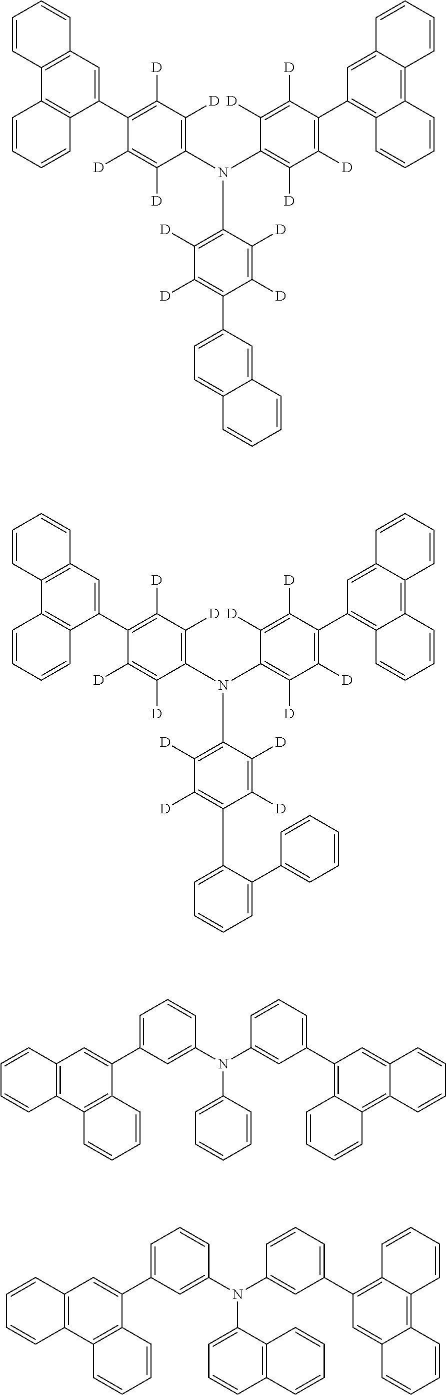 [SMILES: [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1ccc2ccccc2c1 ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1ccccc1-c1ccccc1 ; c1ccc(N(c2cccc(-c3cc4ccccc4c4ccccc34)c2)c2cccc(-c3cc4ccccc4c4ccccc34)c2)cc1 ; c1cc(-c2cc3ccccc3c3ccccc23)cc(N(c2cccc(-c3cc4ccccc4c4ccccc34)c2)c2cccc3ccccc23)c1]

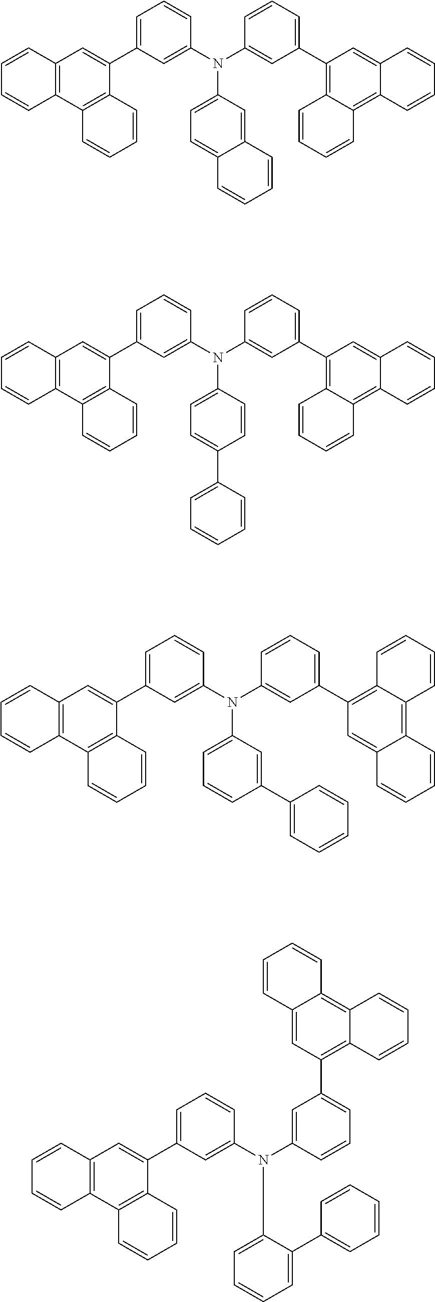 [SMILES: c1cc(-c2cc3ccccc3c3ccccc23)cc(N(c2cccc(-c3cc4ccccc4c4ccccc34)c2)c2ccc3ccccc3c2)c1 ; c1ccc(-c2ccc(N(c3cccc(-c4cc5ccccc5c5ccccc45)c3)c3cccc(-c4cc5ccccc5c5ccccc45)c3)cc2)cc1 ; c1ccc(-c2cccc(N(c3cccc(-c4cc5ccccc5c5ccccc45)c3)c3cccc(-c4cc5ccccc5c5ccccc45)c3)c2)cc1 ; c1ccc(-c2ccccc2N(c2cccc(-c3cc4ccccc4c4ccccc34)c2)c2cccc(-c3cc4ccccc4c4ccccc34)c2)cc1]

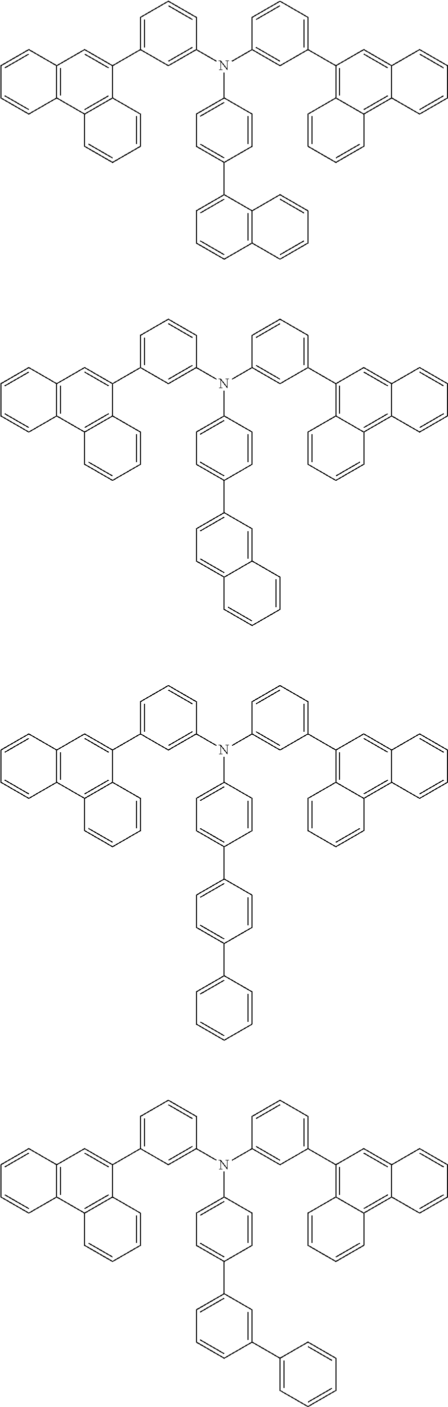 [SMILES: c1cc(-c2cc3ccccc3c3ccccc23)cc(N(c2ccc(-c3cccc4ccccc34)cc2)c2cccc(-c3cc4ccccc4c4ccccc34)c2)c1 ; c1cc(-c2cc3ccccc3c3ccccc23)cc(N(c2ccc(-c3ccc4ccccc4c3)cc2)c2cccc(-c3cc4ccccc4c4ccccc34)c2)c1 ; c1ccc(-c2ccc(-c3ccc(N(c4cccc(-c5cc6ccccc6c6ccccc56)c4)c4cccc(-c5cc6ccccc6c6ccccc56)c4)cc3)cc2)cc1 ; c1ccc(-c2cccc(-c3ccc(N(c4cccc(-c5cc6ccccc6c6ccccc56)c4)c4cccc(-c5cc6ccccc6c6ccccc56)c4)cc3)c2)cc1]

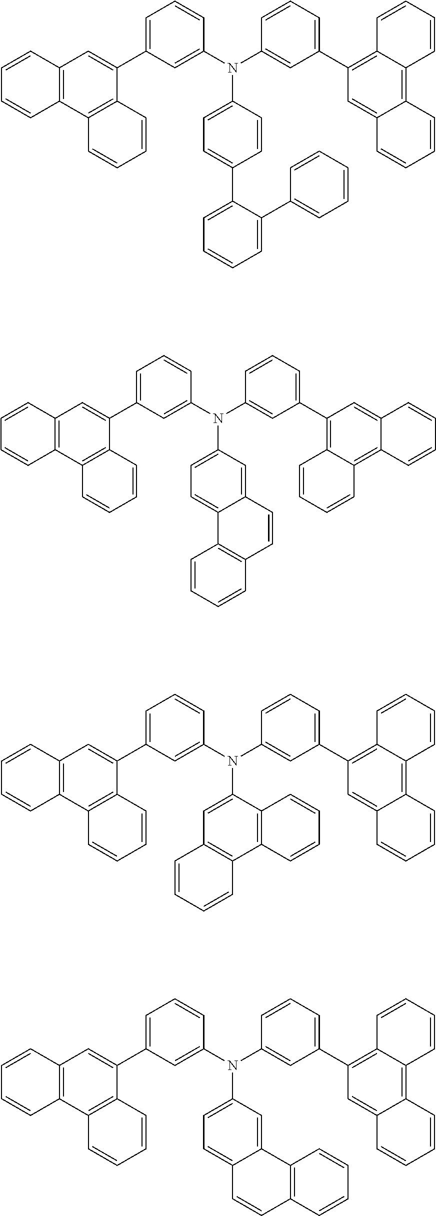 [SMILES: c1ccc(-c2ccccc2-c2ccc(N(c3cccc(-c4cc5ccccc5c5ccccc45)c3)c3cccc(-c4cc5ccccc5c5ccccc45)c3)cc2)cc1 ; c1cc(-c2cc3ccccc3c3ccccc23)cc(N(c2cccc(-c3cc4ccccc4c4ccccc34)c2)c2ccc3c(ccc4ccccc43)c2)c1 ; c1cc(-c2cc3ccccc3c3ccccc23)cc(N(c2cccc(-c3cc4ccccc4c4ccccc34)c2)c2cc3ccccc3c3ccccc23)c1 ; c1cc(-c2cc3ccccc3c3ccccc23)cc(N(c2cccc(-c3cc4ccccc4c4ccccc34)c2)c2ccc3ccc4ccccc4c3c2)c1]

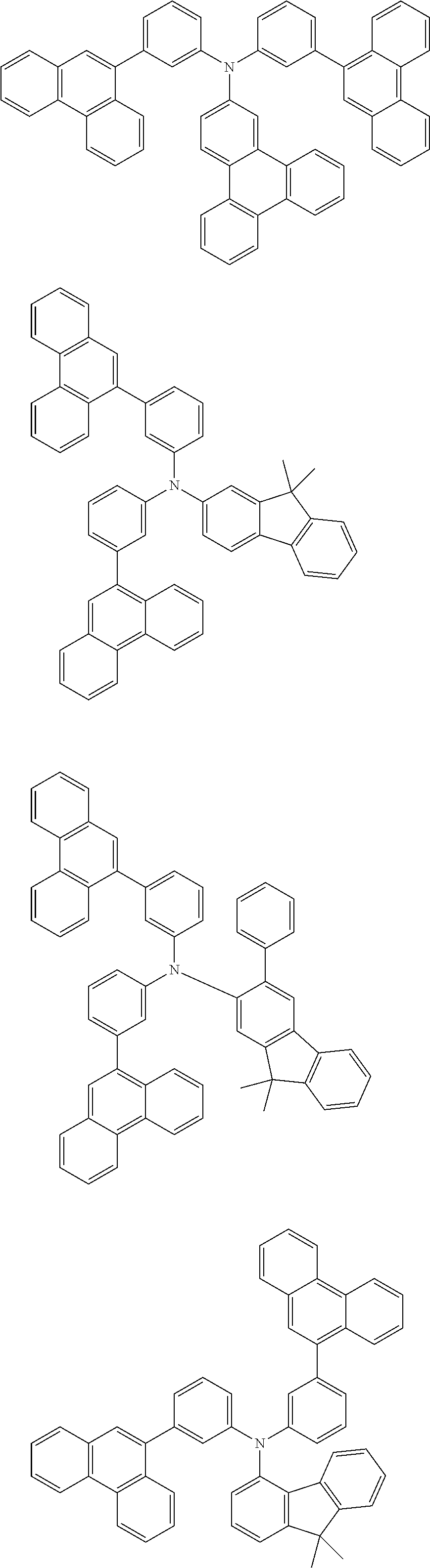 [SMILES: c1cc(-c2cc3ccccc3c3ccccc23)cc(N(c2cccc(-c3cc4ccccc4c4ccccc34)c2)c2ccc3c4ccccc4c4ccccc4c3c2)c1 ; CC1(C)c2ccccc2-c2ccc(N(c3cccc(-c4cc5ccccc5c5ccccc45)c3)c3cccc(-c4cc5ccccc5c5ccccc45)c3)cc21 ; CC1(C)c2ccccc2-c2cc(-c3ccccc3)c(N(c3cccc(-c4cc5ccccc5c5ccccc45)c3)c3cccc(-c4cc5ccccc5c5ccccc45)c3)cc21 ; CC1(C)c2ccccc2-c2c(N(c3cccc(-c4cc5ccccc5c5ccccc45)c3)c3cccc(-c4cc5ccccc5c5ccccc45)c3)cccc21]

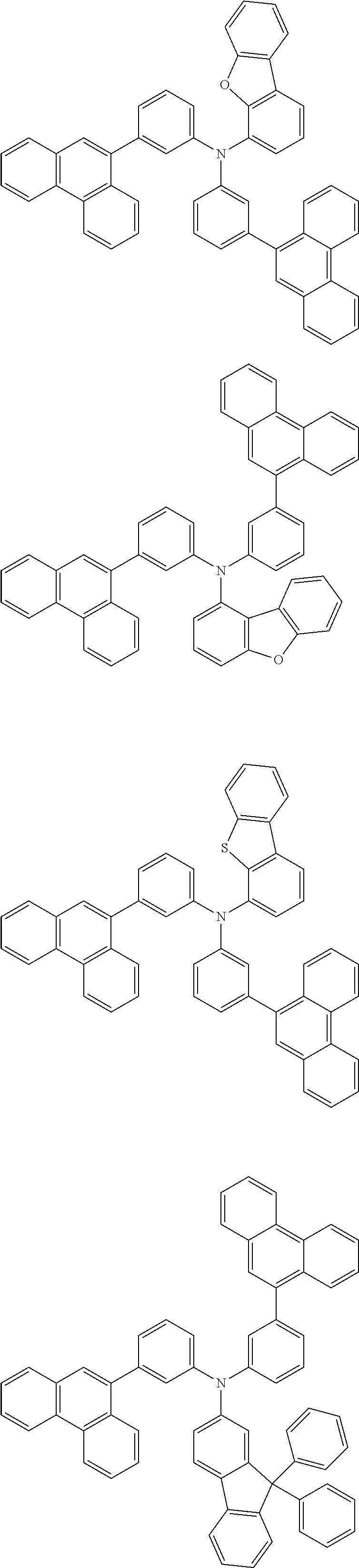 [SMILES: c1cc(-c2cc3ccccc3c3ccccc23)cc(N(c2cccc(-c3cc4ccccc4c4ccccc34)c2)c2cccc3c2oc2ccccc23)c1 ; c1cc(-c2cc3ccccc3c3ccccc23)cc(N(c2cccc(-c3cc4ccccc4c4ccccc34)c2)c2cccc3oc4ccccc4c23)c1 ; c1cc(-c2cc3ccccc3c3ccccc23)cc(N(c2cccc(-c3cc4ccccc4c4ccccc34)c2)c2cccc3c2sc2ccccc23)c1 ; c1ccc(C2(c3ccccc3)c3ccccc3-c3ccc(N(c4cccc(-c5cc6ccccc6c6ccccc56)c4)c4cccc(-c5cc6ccccc6c6ccccc56)c4)cc32)cc1]

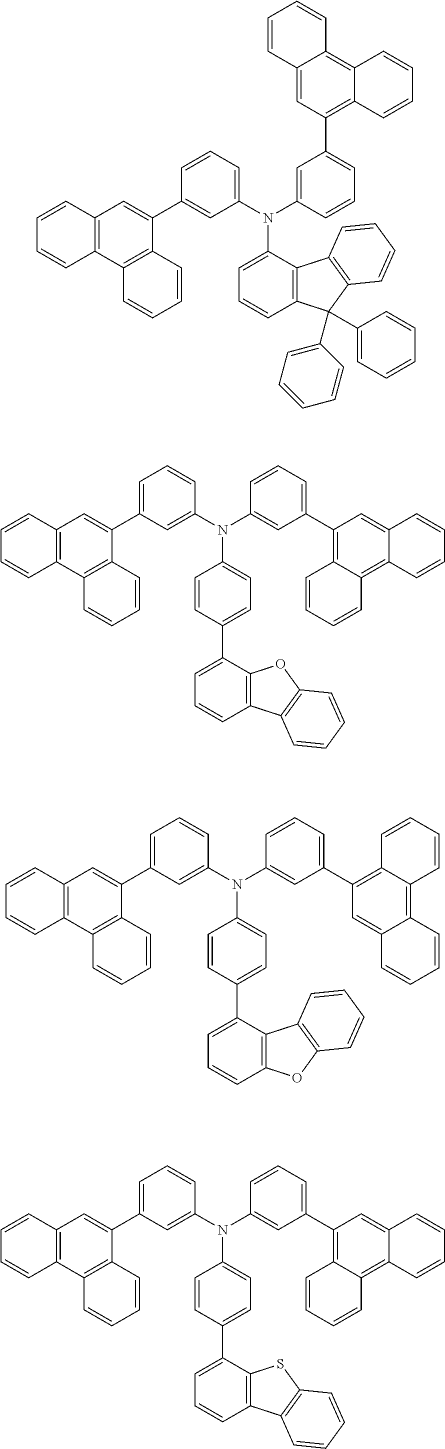 [SMILES: c1ccc(C2(c3ccccc3)c3ccccc3-c3c(N(c4cccc(-c5cc6ccccc6c6ccccc56)c4)c4cccc(-c5cc6ccccc6c6ccccc56)c4)cccc32)cc1 ; c1cc(-c2cc3ccccc3c3ccccc23)cc(N(c2ccc(-c3cccc4c3oc3ccccc34)cc2)c2cccc(-c3cc4ccccc4c4ccccc34)c2)c1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1 ; c1cc(-c2cc3ccccc3c3ccccc23)cc(N(c2ccc(-c3cccc4c3sc3ccccc34)cc2)c2cccc(-c3cc4ccccc4c4ccccc34)c2)c1]

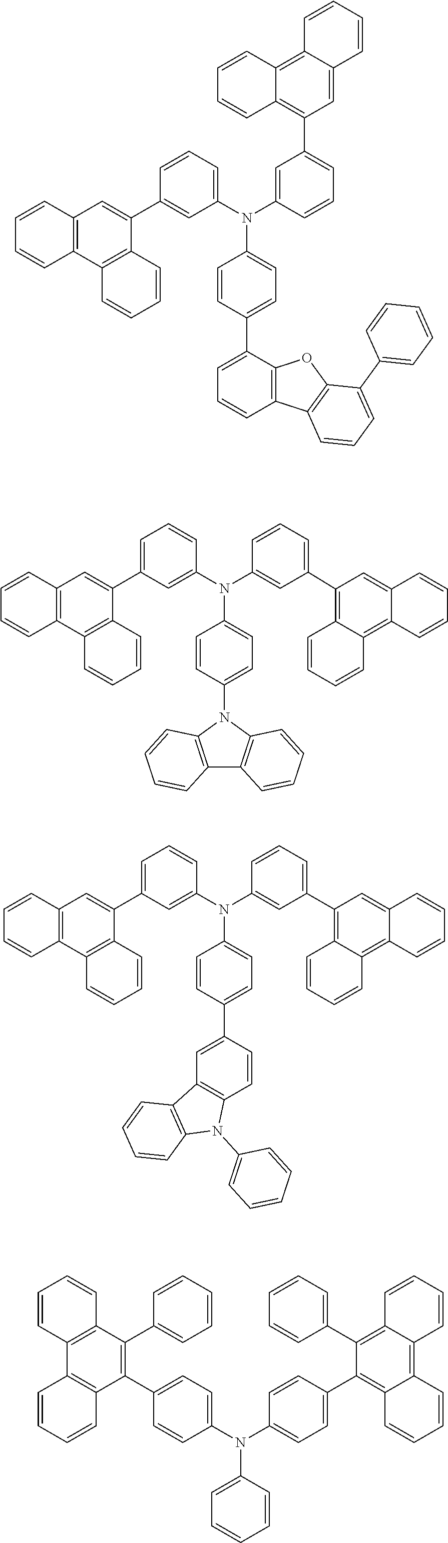 [SMILES: c1ccc(-c2cccc3c2oc2c(-c4ccc(N(c5cccc(-c6cc7ccccc7c7ccccc67)c5)c5cccc(-c6cc7ccccc7c7ccccc67)c5)cc4)cccc23)cc1 ; c1cc(-c2cc3ccccc3c3ccccc23)cc(N(c2ccc(-n3c4ccccc4c4ccccc43)cc2)c2cccc(-c3cc4ccccc4c4ccccc34)c2)c1 ; c1ccc(-n2c3ccccc3c3cc(-c4ccc(N(c5cccc(-c6cc7ccccc7c7ccccc67)c5)c5cccc(-c6cc7ccccc7c7ccccc67)c5)cc4)ccc32)cc1 ; c1ccc(-c2c(-c3ccc(N(c4ccccc4)c4ccc(-c5c(-c6ccccc6)c6ccccc6c6ccccc56)cc4)cc3)c3ccccc3c3ccccc23)cc1]

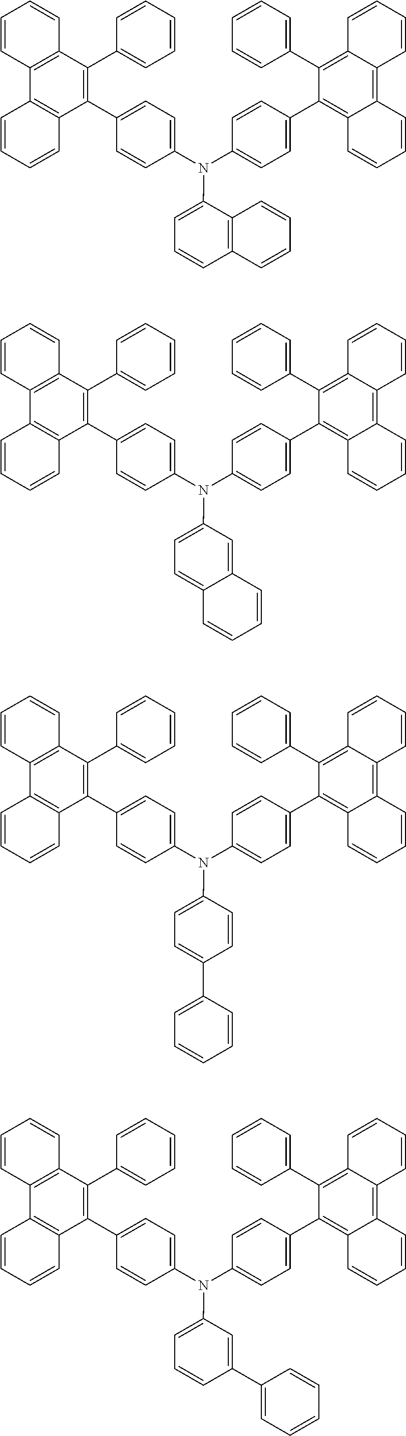 [SMILES: c1ccc(-c2c(-c3ccc(N(c4ccc(-c5c(-c6ccccc6)c6ccccc6c6ccccc56)cc4)c4cccc5ccccc45)cc3)c3ccccc3c3ccccc23)cc1 ; c1ccc(-c2c(-c3ccc(N(c4ccc(-c5c(-c6ccccc6)c6ccccc6c6ccccc56)cc4)c4ccc5ccccc5c4)cc3)c3ccccc3c3ccccc23)cc1 ; c1ccc(-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1 ; c1ccc(-c2cccc(N(c3ccc(-c4c(-c5ccccc5)c5ccccc5c5ccccc45)cc3)c3ccc(-c4c(-c5ccccc5)c5ccccc5c5ccccc45)cc3)c2)cc1]

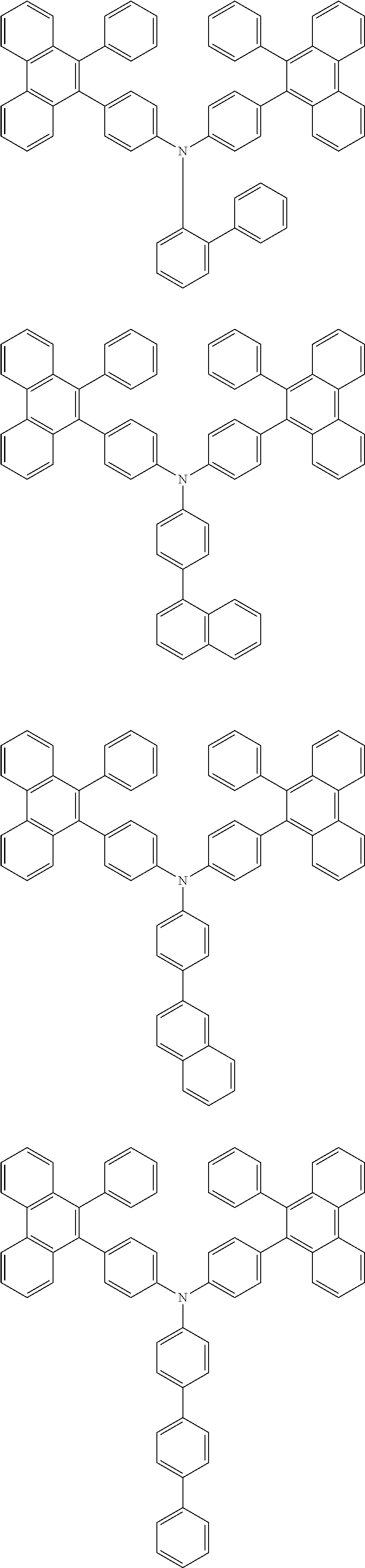 [SMILES: c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1 ; c1ccc(-c2c(-c3ccc(N(c4ccc(-c5cccc6ccccc56)cc4)c4ccc(-c5c(-c6ccccc6)c6ccccc6c6ccccc56)cc4)cc3)c3ccccc3c3ccccc23)cc1 ; c1ccc(-c2c(-c3ccc(N(c4ccc(-c5ccc6ccccc6c5)cc4)c4ccc(-c5c(-c6ccccc6)c6ccccc6c6ccccc56)cc4)cc3)c3ccccc3c3ccccc23)cc1 ; c1ccc(-c2ccc(-c3ccc(N(c4ccc(-c5c(-c6ccccc6)c6ccccc6c6ccccc56)cc4)c4ccc(-c5c(-c6ccccc6)c6ccccc6c6ccccc56)cc4)cc3)cc2)cc1]

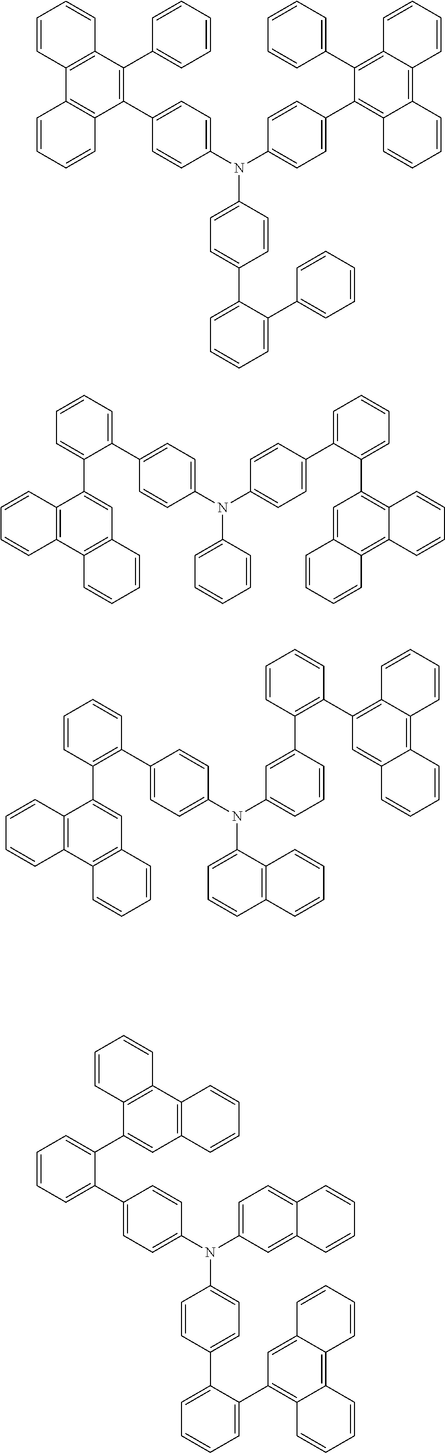 [SMILES: c1ccc(-c2ccccc2-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1 ; c1ccc(N(c2ccc(-c3ccccc3-c3cc4ccccc4c4ccccc34)cc2)c2ccc(-c3ccccc3-c3cc4ccccc4c4ccccc34)cc2)cc1 ; c1cc(-c2ccccc2-c2cc3ccccc3c3ccccc23)cc(N(c2ccc(-c3ccccc3-c3cc4ccccc4c4ccccc34)cc2)c2cccc3ccccc23)c1 ; c1ccc(-c2cc3ccccc3c3ccccc23)c(-c2ccc(N(c3ccc(-c4ccccc4-c4cc5ccccc5c5ccccc45)cc3)c3ccc4ccccc4c3)cc2)c1]

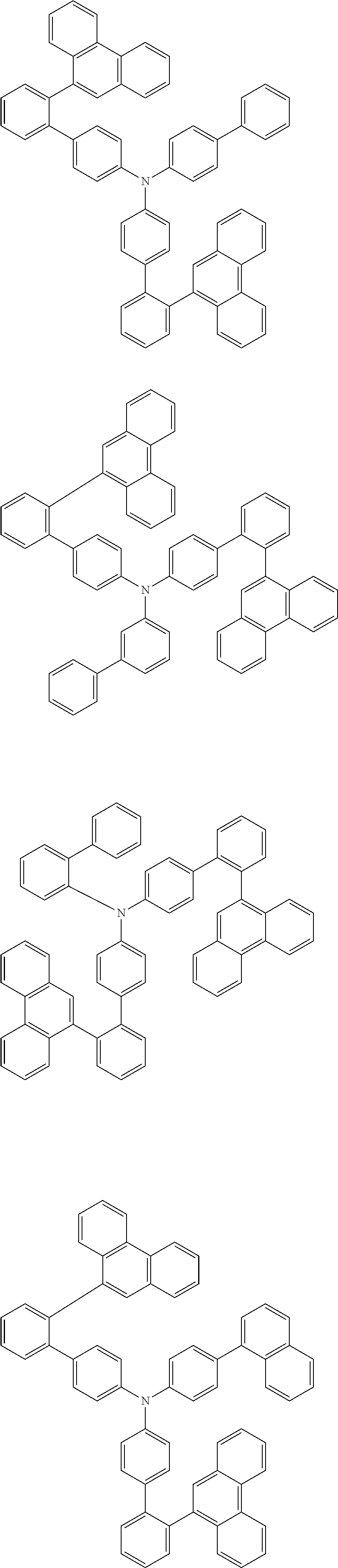 [SMILES: c1ccc(-c2ccc(N(c3ccc(-c4ccccc4-c4cc5ccccc5c5ccccc45)cc3)c3ccc(-c4ccccc4-c4cc5ccccc5c5ccccc45)cc3)cc2)cc1 ; c1ccc(-c2cccc(N(c3ccc(-c4ccccc4-c4cc5ccccc5c5ccccc45)cc3)c3ccc(-c4ccccc4-c4cc5ccccc5c5ccccc45)cc3)c2)cc1 ; c1ccc(-c2ccccc2N(c2ccc(-c3ccccc3-c3cc4ccccc4c4ccccc34)cc2)c2ccc(-c3ccccc3-c3cc4ccccc4c4ccccc34)cc2)cc1 ; c1ccc(-c2cc3ccccc3c3ccccc23)c(-c2ccc(N(c3ccc(-c4ccccc4-c4cc5ccccc5c5ccccc45)cc3)c3ccc(-c4cccc5ccccc45)cc3)cc2)c1]

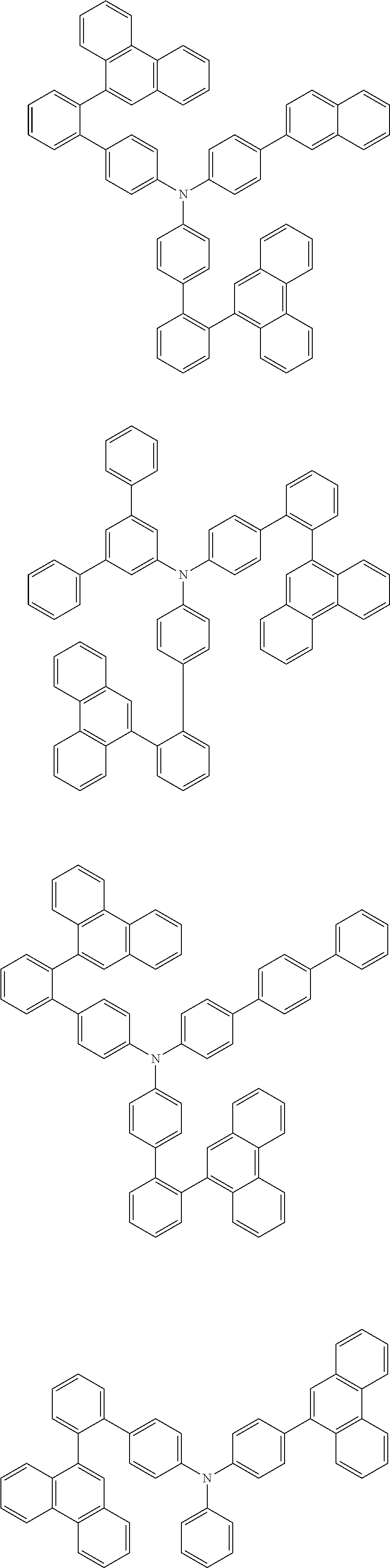 [SMILES: c1ccc(-c2cc3ccccc3c3ccccc23)c(-c2ccc(N(c3ccc(-c4ccc5ccccc5c4)cc3)c3ccc(-c4ccccc4-c4cc5ccccc5c5ccccc45)cc3)cc2)c1 ; c1ccc(-c2cc(-c3ccccc3)cc(N(c3ccc(-c4ccccc4-c4cc5ccccc5c5ccccc45)cc3)c3ccc(-c4ccccc4-c4cc5ccccc5c5ccccc45)cc3)c2)cc1 ; c1ccc(-c2ccc(-c3ccc(N(c4ccc(-c5ccccc5-c5cc6ccccc6c6ccccc56)cc4)c4ccc(-c5ccccc5-c5cc6ccccc6c6ccccc56)cc4)cc3)cc2)cc1 ; c1ccc(N(c2ccc(-c3ccccc3-c3cc4ccccc4c4ccccc34)cc2)c2ccc(-c3cc4ccccc4c4ccccc34)cc2)cc1]

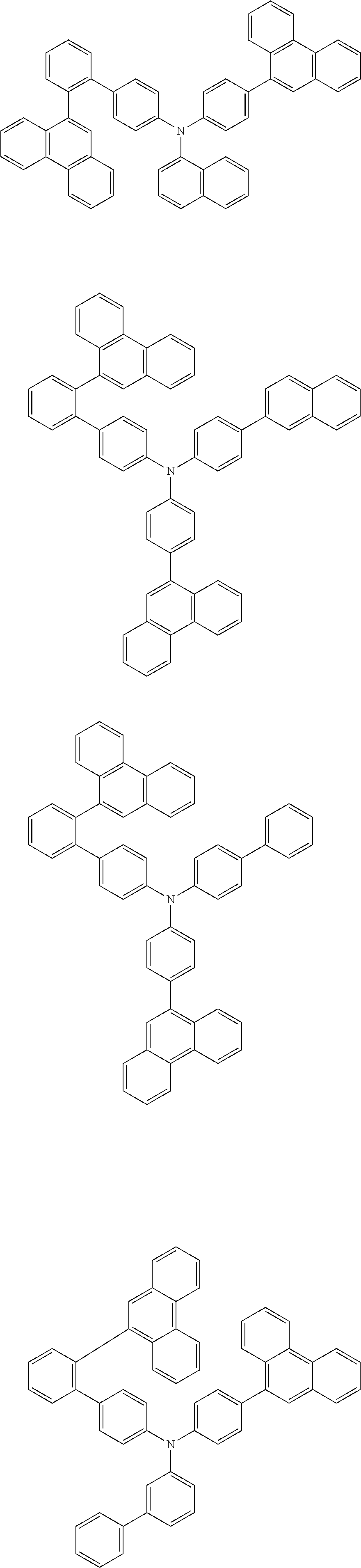 [SMILES: c1ccc(-c2cc3ccccc3c3ccccc23)c(-c2ccc(N(c3ccc(-c4cc5ccccc5c5ccccc45)cc3)c3cccc4ccccc34)cc2)c1 ; c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1 ; c1ccc(-c2ccc(N(c3ccc(-c4ccccc4-c4cc5ccccc5c5ccccc45)cc3)c3ccc(-c4cc5ccccc5c5ccccc45)cc3)cc2)cc1 ; c1ccc(-c2cccc(N(c3ccc(-c4ccccc4-c4cc5ccccc5c5ccccc45)cc3)c3ccc(-c4cc5ccccc5c5ccccc45)cc3)c2)cc1]

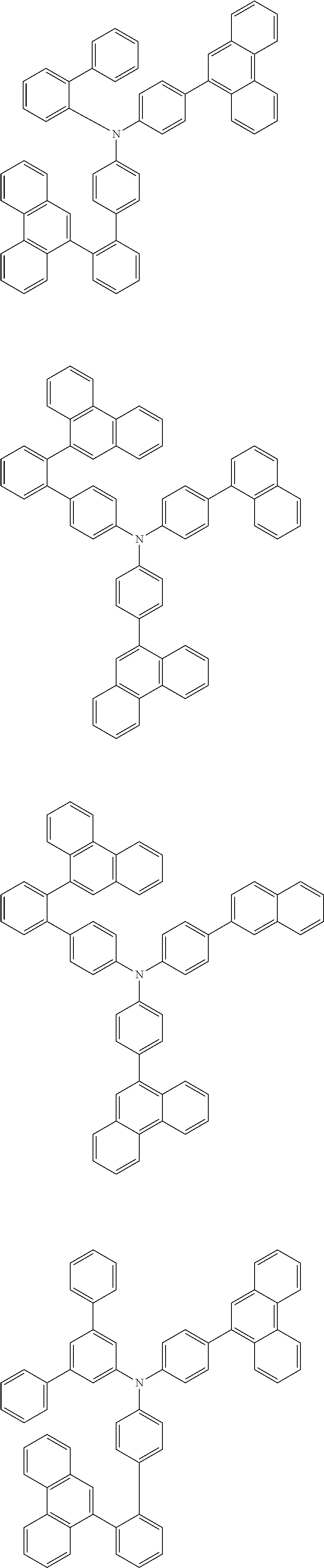 [SMILES: c1ccc(-c2ccccc2N(c2ccc(-c3ccccc3-c3cc4ccccc4c4ccccc34)cc2)c2ccc(-c3cc4ccccc4c4ccccc34)cc2)cc1 ; c1ccc(-c2cc3ccccc3c3ccccc23)c(-c2ccc(N(c3ccc(-c4cccc5ccccc45)cc3)c3ccc(-c4cc5ccccc5c5ccccc45)cc3)cc2)c1 ; c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1 ; c1ccc(-c2cc(-c3ccccc3)cc(N(c3ccc(-c4ccccc4-c4cc5ccccc5c5ccccc45)cc3)c3ccc(-c4cc5ccccc5c5ccccc45)cc3)c2)cc1]

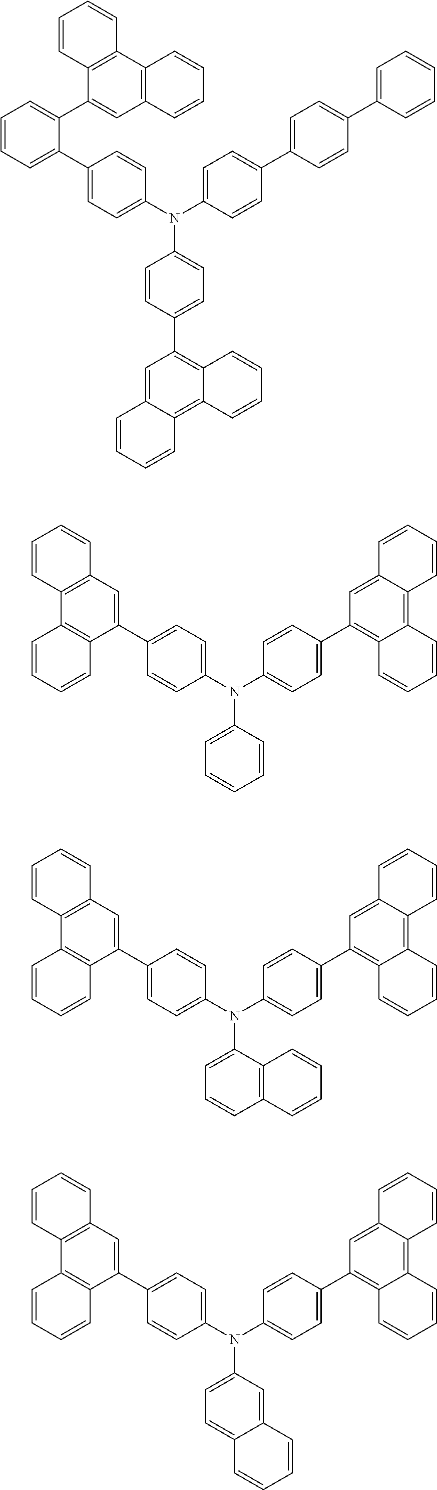 [SMILES: c1ccc(-c2ccc(-c3ccc(N(c4ccc(-c5ccccc5-c5cc6ccccc6c6ccccc56)cc4)c4ccc(-c5cc6ccccc6c6ccccc56)cc4)cc3)cc2)cc1 ; c1ccc(N(c2ccc(-c3cc4ccccc4c4ccccc34)cc2)c2ccc(-c3cc4ccccc4c4ccccc34)cc2)cc1 ; c1ccc2c(N(c3ccc(-c4cc5ccccc5c5ccccc45)cc3)c3ccc(-c4cc5ccccc5c5ccccc45)cc3)cccc2c1 ; c1ccc2cc(N(c3ccc(-c4cc5ccccc5c5ccccc45)cc3)c3ccc(-c4cc5ccccc5c5ccccc45)cc3)ccc2c1]

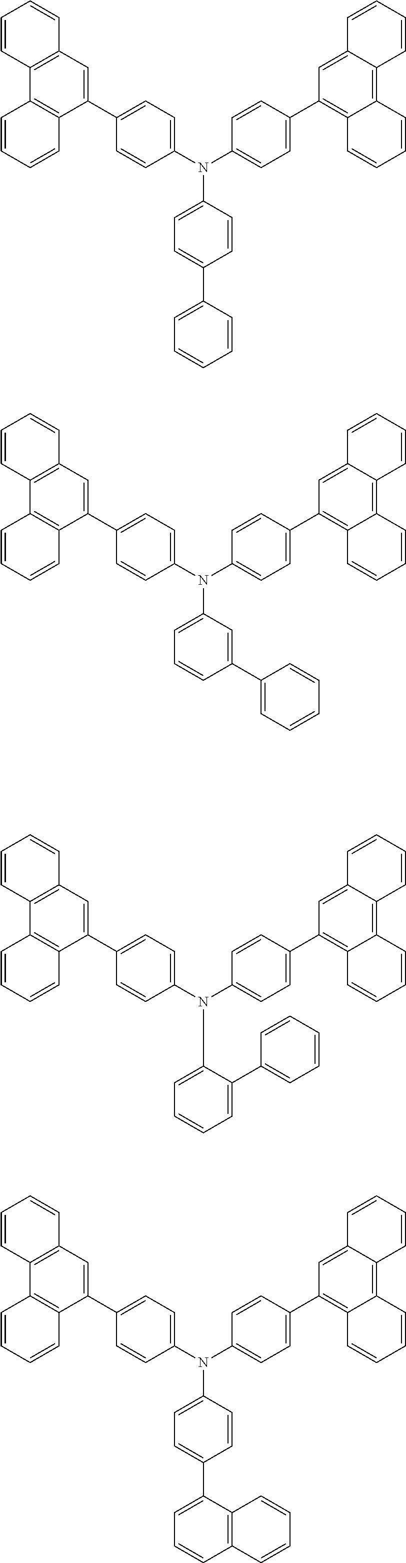 [SMILES: c1ccc(-c2ccc(N(c3ccc(-c4cc5ccccc5c5ccccc45)cc3)c3ccc(-c4cc5ccccc5c5ccccc45)cc3)cc2)cc1 ; c1ccc(-c2cccc(N(c3ccc(-c4cc5ccccc5c5ccccc45)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cc4ccccc4c4ccccc34)cc2)cc1 ; c1ccc2c(-c3ccc(N(c4ccc(-c5cc6ccccc6c6ccccc56)cc4)c4ccc(-c5cc6ccccc6c6ccccc56)cc4)cc3)cccc2c1]

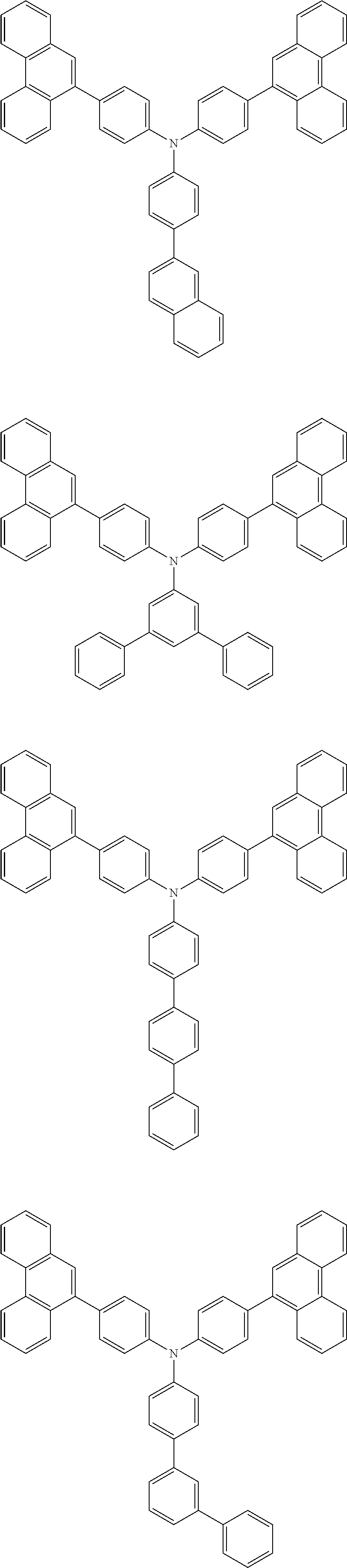 [SMILES: c1ccc2cc(-c3ccc(N(c4ccc(-c5cc6ccccc6c6ccccc56)cc4)c4ccc(-c5cc6ccccc6c6ccccc56)cc4)cc3)ccc2c1 ; c1ccc(-c2cc(-c3ccccc3)cc(N(c3ccc(-c4cc5ccccc5c5ccccc45)cc3)c3ccc(-c4cc5ccccc5c5ccccc45)cc3)c2)cc1 ; c1ccc(-c2ccc(-c3ccc(N(c4ccc(-c5cc6ccccc6c6ccccc56)cc4)c4ccc(-c5cc6ccccc6c6ccccc56)cc4)cc3)cc2)cc1 ; c1ccc(-c2cccc(-c3ccc(N(c4ccc(-c5cc6ccccc6c6ccccc56)cc4)c4ccc(-c5cc6ccccc6c6ccccc56)cc4)cc3)c2)cc1]

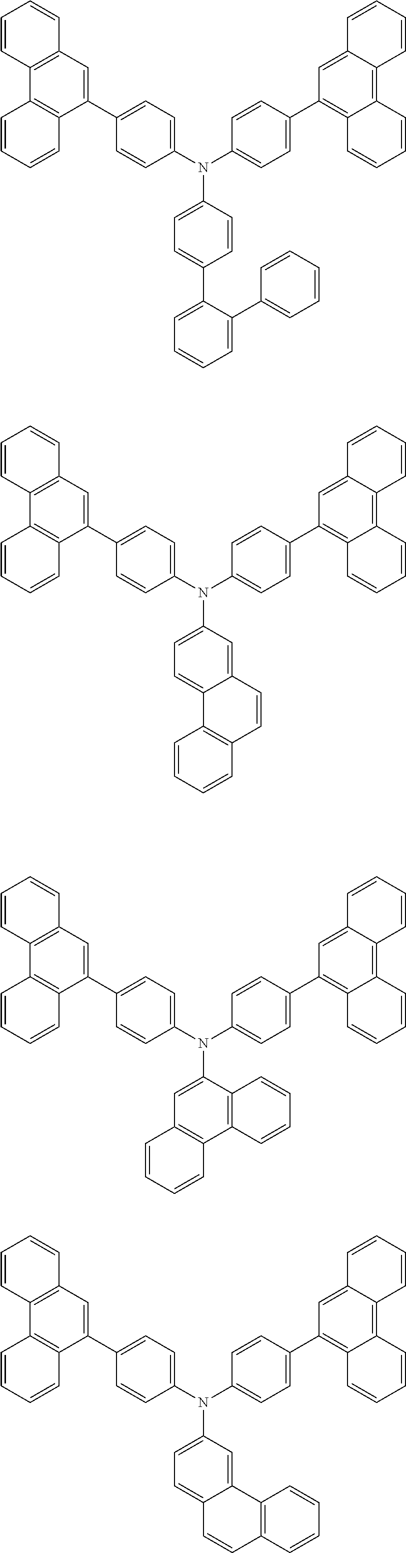 [SMILES: c1ccc(-c2ccccc2-c2ccc(N(c3ccc(-c4cc5ccccc5c5ccccc45)cc3)c3ccc(-c4cc5ccccc5c5ccccc45)cc3)cc2)cc1 ; c1ccc2c(c1)ccc1cc(N(c3ccc(-c4cc5ccccc5c5ccccc45)cc3)c3ccc(-c4cc5ccccc5c5ccccc45)cc3)ccc12 ; c1ccc2c(c1)cc(-c1ccc(N(c3ccc(-c4cc5ccccc5c5ccccc45)cc3)c3cc4ccccc4c4ccccc34)cc1)c1ccccc12 ; c1ccc2c(c1)ccc1ccc(N(c3ccc(-c4cc5ccccc5c5ccccc45)cc3)c3ccc(-c4cc5ccccc5c5ccccc45)cc3)cc12]

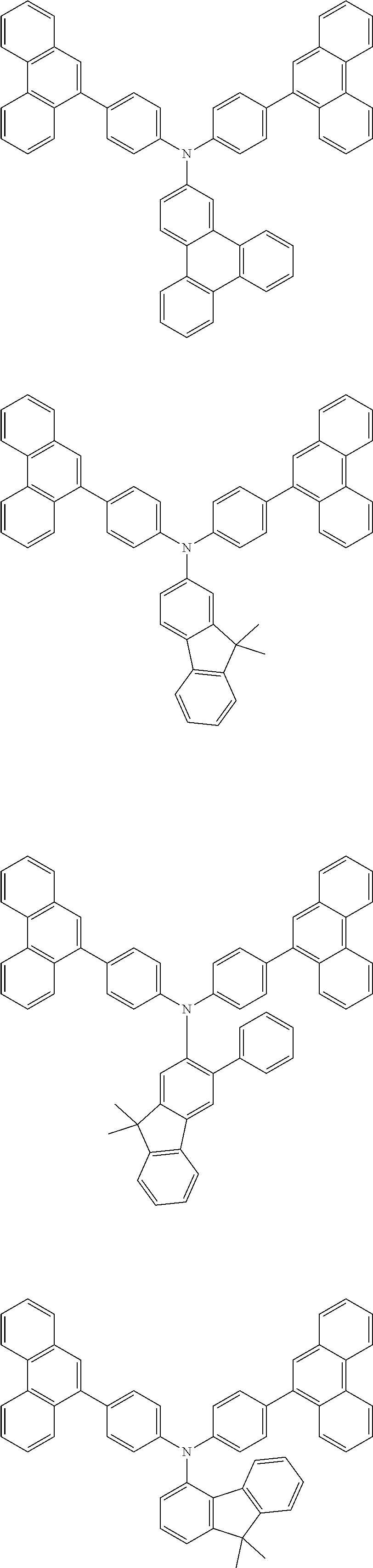 [SMILES: c1ccc2c(c1)cc(-c1ccc(N(c3ccc(-c4cc5ccccc5c5ccccc45)cc3)c3ccc4c5ccccc5c5ccccc5c4c3)cc1)c1ccccc12 ; CC1(C)c2ccccc2-c2ccc(N(c3ccc(-c4cc5ccccc5c5ccccc45)cc3)c3ccc(-c4cc5ccccc5c5ccccc45)cc3)cc21 ; CC1(C)c2ccccc2-c2cc(-c3ccccc3)c(N(c3ccc(-c4cc5ccccc5c5ccccc45)cc3)c3ccc(-c4cc5ccccc5c5ccccc45)cc3)cc21 ; CC1(C)c2ccccc2-c2c(N(c3ccc(-c4cc5ccccc5c5ccccc45)cc3)c3ccc(-c4cc5ccccc5c5ccccc45)cc3)cccc21]

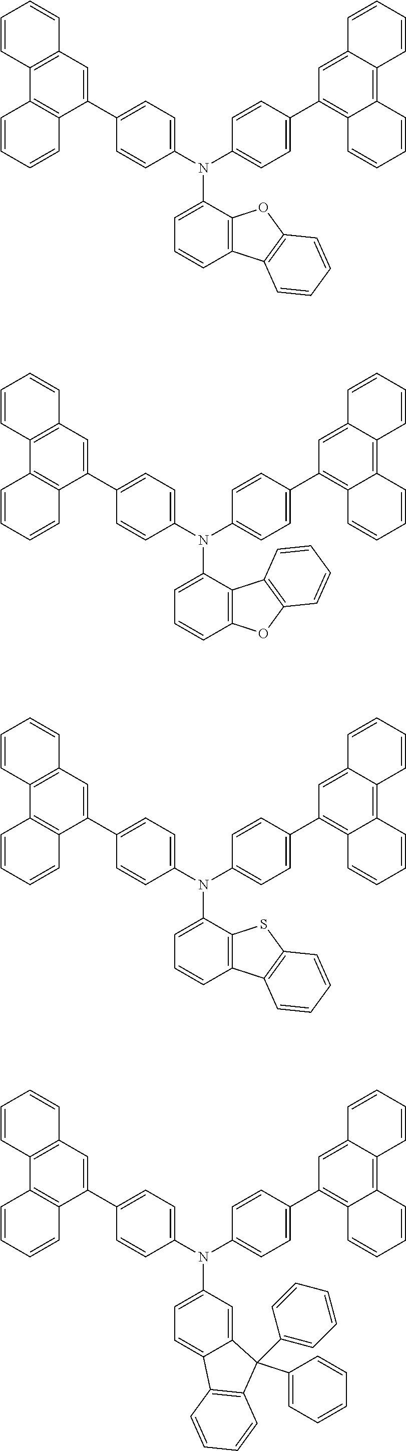 [SMILES: c1ccc2c(c1)cc(-c1ccc(N(c3ccc(-c4cc5ccccc5c5ccccc45)cc3)c3cccc4c3oc3ccccc34)cc1)c1ccccc12 ; c1ccc2c(c1)cc(-c1ccc(N(c3ccc(-c4cc5ccccc5c5ccccc45)cc3)c3cccc4oc5ccccc5c34)cc1)c1ccccc12 ; c1ccc2c(c1)cc(-c1ccc(N(c3ccc(-c4cc5ccccc5c5ccccc45)cc3)c3cccc4c3sc3ccccc34)cc1)c1ccccc12 ; c1ccc(C2(c3ccccc3)c3ccccc3-c3ccc(N(c4ccc(-c5cc6ccccc6c6ccccc56)cc4)c4ccc(-c5cc6ccccc6c6ccccc56)cc4)cc32)cc1]

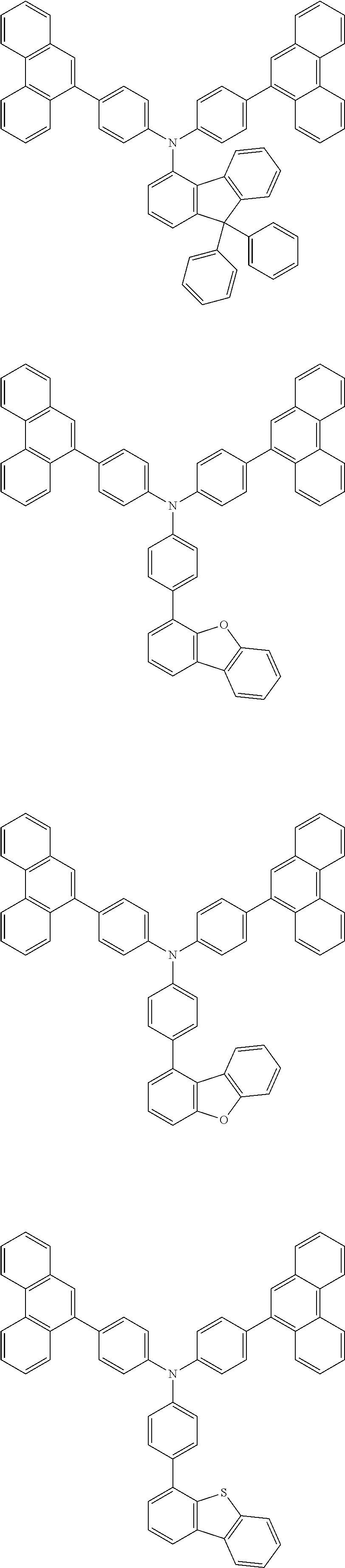 [SMILES: c1ccc(C2(c3ccccc3)c3ccccc3-c3c(N(c4ccc(-c5cc6ccccc6c6ccccc56)cc4)c4ccc(-c5cc6ccccc6c6ccccc56)cc4)cccc32)cc1 ; c1ccc2c(c1)cc(-c1ccc(N(c3ccc(-c4cc5ccccc5c5ccccc45)cc3)c3ccc(-c4cccc5c4oc4ccccc45)cc3)cc1)c1ccccc12 ; c1ccc2c(c1)cc(-c1ccc(N(c3ccc(-c4cc5ccccc5c5ccccc45)cc3)c3ccc(-c4cccc5oc6ccccc6c45)cc3)cc1)c1ccccc12 ; c1ccc2c(c1)cc(-c1ccc(N(c3ccc(-c4cc5ccccc5c5ccccc45)cc3)c3ccc(-c4cccc5c4sc4ccccc45)cc3)cc1)c1ccccc12]

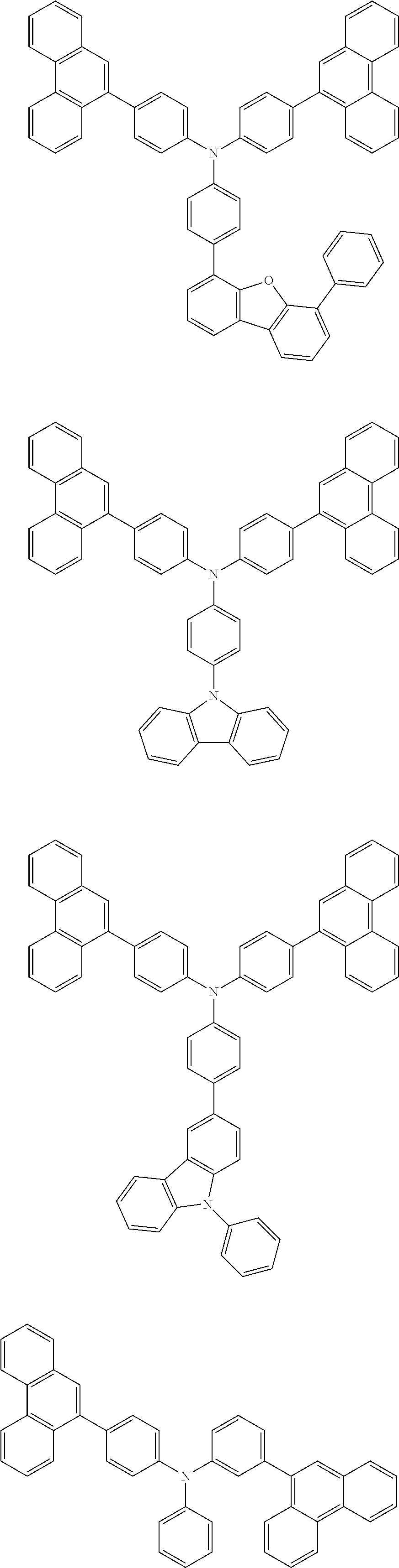 [SMILES: c1ccc(-c2cccc3c2oc2c(-c4ccc(N(c5ccc(-c6cc7ccccc7c7ccccc67)cc5)c5ccc(-c6cc7ccccc7c7ccccc67)cc5)cc4)cccc23)cc1 ; c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12 ; c1ccc(-n2c3ccccc3c3cc(-c4ccc(N(c5ccc(-c6cc7ccccc7c7ccccc67)cc5)c5ccc(-c6cc7ccccc7c7ccccc67)cc5)cc4)ccc32)cc1 ; c1ccc(N(c2ccc(-c3cc4ccccc4c4ccccc34)cc2)c2cccc(-c3cc4ccccc4c4ccccc34)c2)cc1]

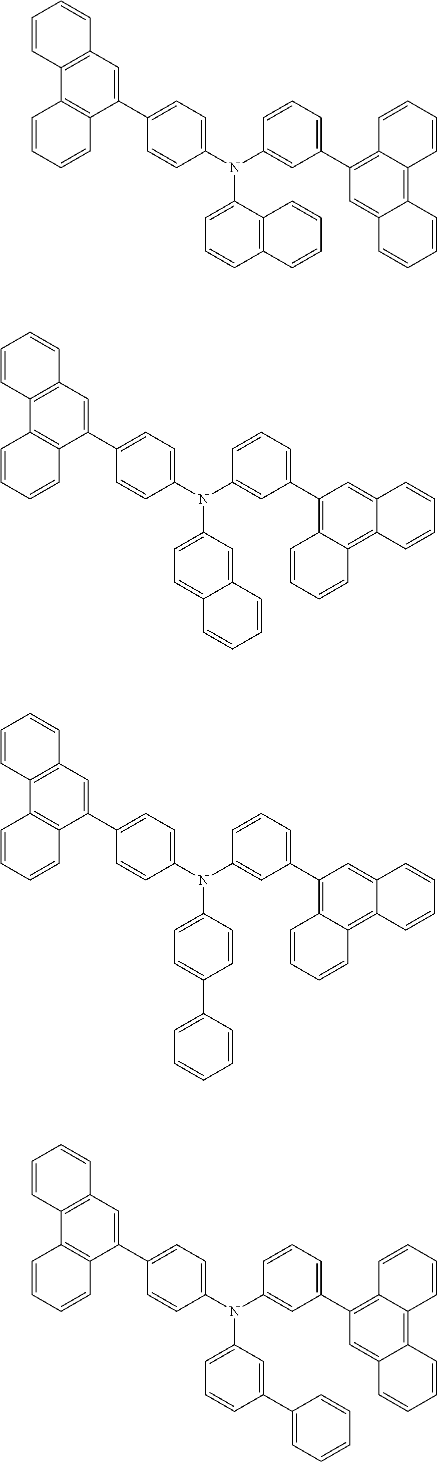 [SMILES: c1cc(-c2cc3ccccc3c3ccccc23)cc(N(c2ccc(-c3cc4ccccc4c4ccccc34)cc2)c2cccc3ccccc23)c1 ; c1cc(-c2cc3ccccc3c3ccccc23)cc(N(c2ccc(-c3cc4ccccc4c4ccccc34)cc2)c2ccc3ccccc3c2)c1 ; c1ccc(-c2ccc(N(c3ccc(-c4cc5ccccc5c5ccccc45)cc3)c3cccc(-c4cc5ccccc5c5ccccc45)c3)cc2)cc1 ; c1ccc(-c2cccc(N(c3ccc(-c4cc5ccccc5c5ccccc45)cc3)c3cccc(-c4cc5ccccc5c5ccccc45)c3)c2)cc1]

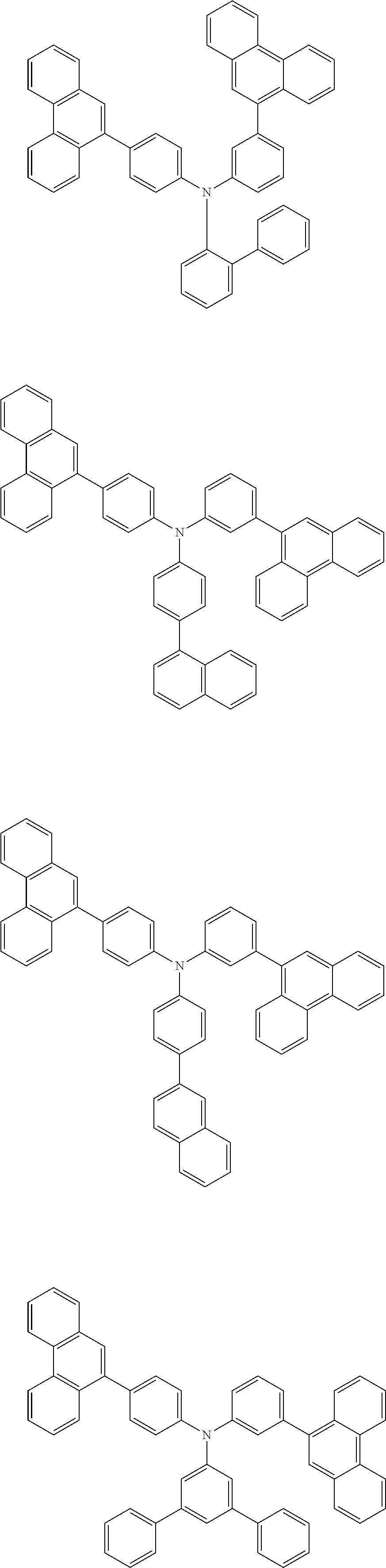 [SMILES: c1ccc(-c2ccccc2N(c2ccc(-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3cccc4ccccc34)cc2)c2ccc(-c3cc4ccccc4c4ccccc34)cc2)c1 ; c1cc(-c2cc3ccccc3c3ccccc23)cc(N(c2ccc(-c3ccc4ccccc4c3)cc2)c2ccc(-c3cc4ccccc4c4ccccc34)cc2)c1 ; c1ccc(-c2cc(-c3ccccc3)cc(N(c3ccc(-c4cc5ccccc5c5ccccc45)cc3)c3cccc(-c4cc5ccccc5c5ccccc45)c3)c2)cc1]

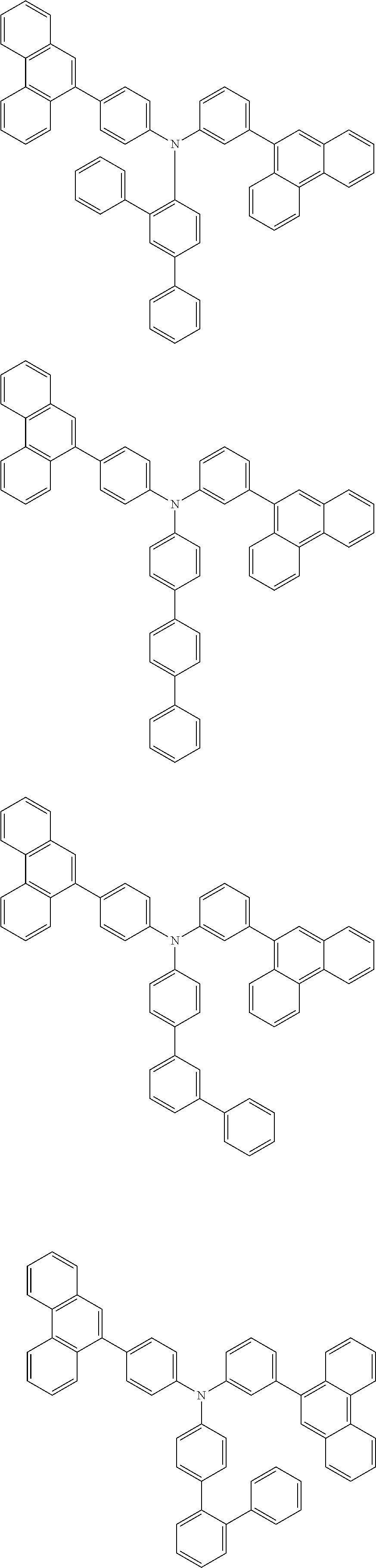 [SMILES: c1ccc(-c2ccc(N(c3ccc(-c4cc5ccccc5c5ccccc45)cc3)c3cccc(-c4cc5ccccc5c5ccccc45)c3)c(-c3ccccc3)c2)cc1 ; c1ccc(-c2ccc(-c3ccc(N(c4ccc(-c5cc6ccccc6c6ccccc56)cc4)c4cccc(-c5cc6ccccc6c6ccccc56)c4)cc3)cc2)cc1 ; c1ccc(-c2cccc(-c3ccc(N(c4ccc(-c5cc6ccccc6c6ccccc56)cc4)c4cccc(-c5cc6ccccc6c6ccccc56)c4)cc3)c2)cc1 ; c1ccc(-c2ccccc2-c2ccc(N(c3ccc(-c4cc5ccccc5c5ccccc45)cc3)c3cccc(-c4cc5ccccc5c5ccccc45)c3)cc2)cc1]

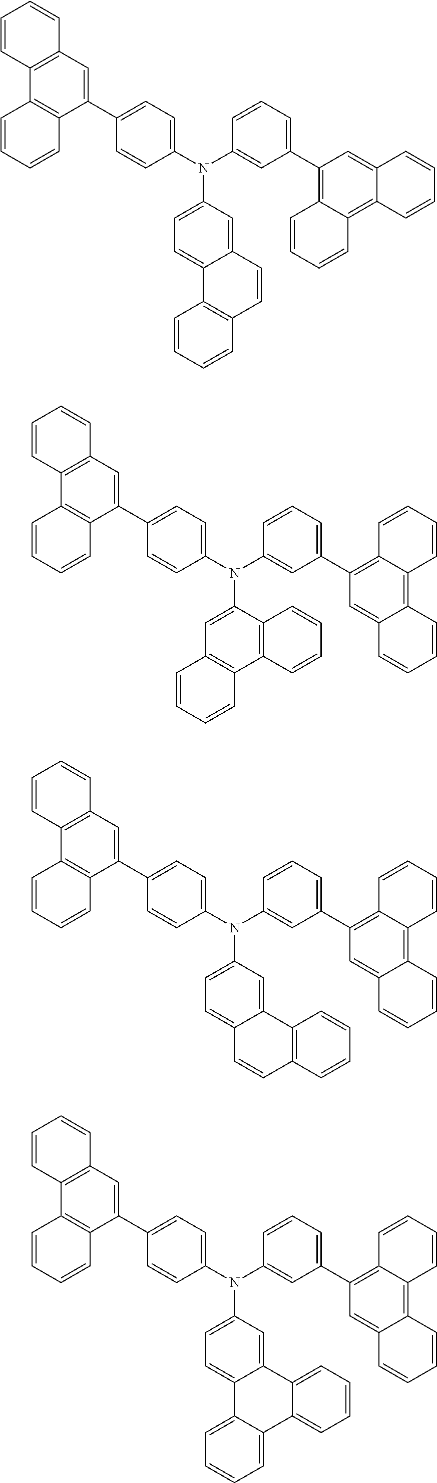 [SMILES: c1cc(-c2cc3ccccc3c3ccccc23)cc(N(c2ccc(-c3cc4ccccc4c4ccccc34)cc2)c2ccc3c(ccc4ccccc43)c2)c1 ; c1cc(-c2cc3ccccc3c3ccccc23)cc(N(c2ccc(-c3cc4ccccc4c4ccccc34)cc2)c2cc3ccccc3c3ccccc23)c1 ; c1cc(-c2cc3ccccc3c3ccccc23)cc(N(c2ccc(-c3cc4ccccc4c4ccccc34)cc2)c2ccc3ccc4ccccc4c3c2)c1 ; c1cc(-c2cc3ccccc3c3ccccc23)cc(N(c2ccc(-c3cc4ccccc4c4ccccc34)cc2)c2ccc3c4ccccc4c4ccccc4c3c2)c1]

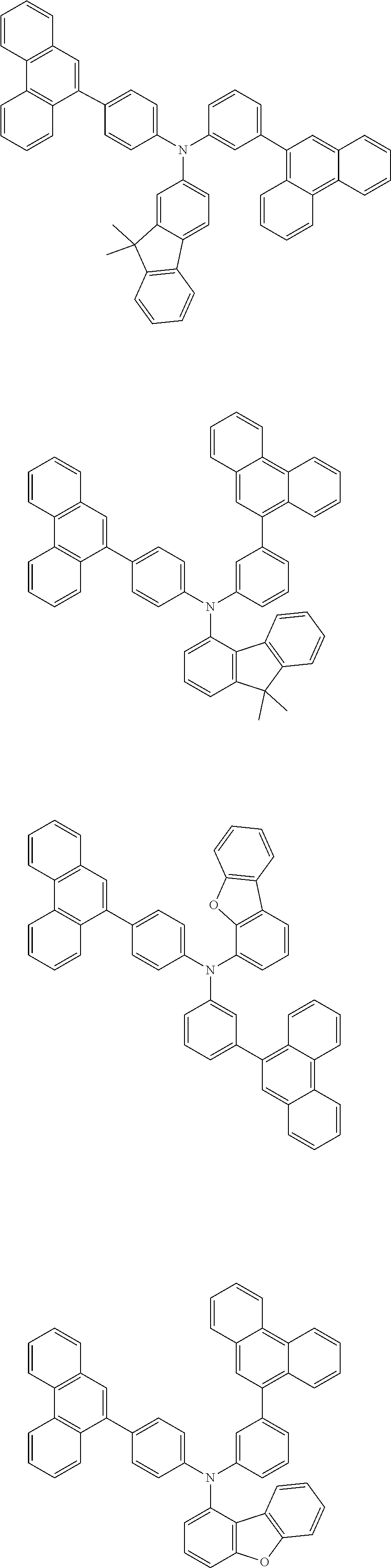 [SMILES: CC1(C)c2ccccc2-c2ccc(N(c3ccc(-c4cc5ccccc5c5ccccc45)cc3)c3cccc(-c4cc5ccccc5c5ccccc45)c3)cc21 ; CC1(C)c2ccccc2-c2c(N(c3ccc(-c4cc5ccccc5c5ccccc45)cc3)c3cccc(-c4cc5ccccc5c5ccccc45)c3)cccc21 ; c1cc(-c2cc3ccccc3c3ccccc23)cc(N(c2ccc(-c3cc4ccccc4c4ccccc34)cc2)c2cccc3c2oc2ccccc23)c1 ; c1cc(-c2cc3ccccc3c3ccccc23)cc(N(c2ccc(-c3cc4ccccc4c4ccccc34)cc2)c2cccc3oc4ccccc4c23)c1]

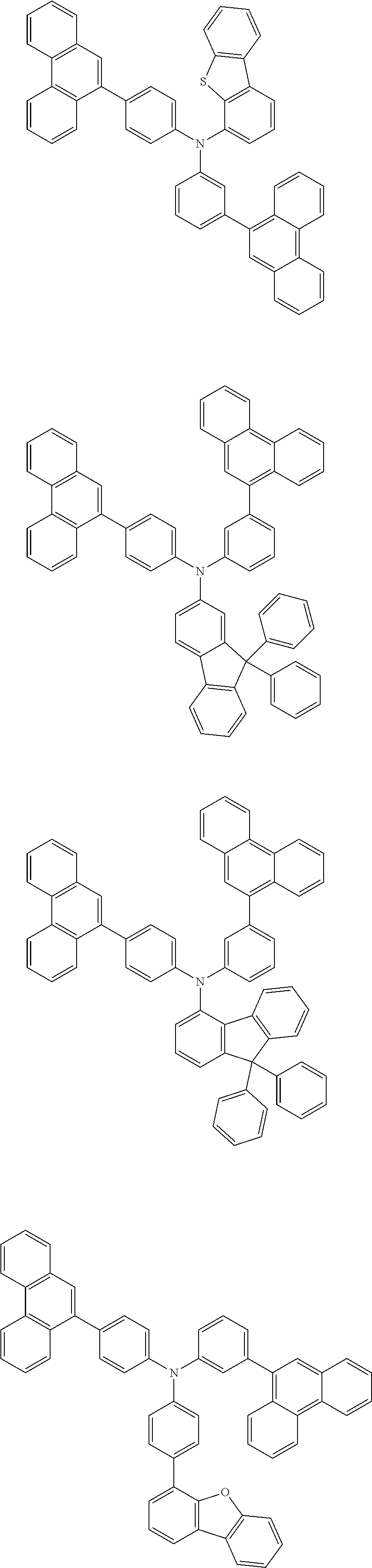 [SMILES: c1cc(-c2cc3ccccc3c3ccccc23)cc(N(c2ccc(-c3cc4ccccc4c4ccccc34)cc2)c2cccc3c2sc2ccccc23)c1 ; c1ccc(C2(c3ccccc3)c3ccccc3-c3ccc(N(c4ccc(-c5cc6ccccc6c6ccccc56)cc4)c4cccc(-c5cc6ccccc6c6ccccc56)c4)cc32)cc1 ; c1ccc(C2(c3ccccc3)c3ccccc3-c3c(N(c4ccc(-c5cc6ccccc6c6ccccc56)cc4)c4cccc(-c5cc6ccccc6c6ccccc56)c4)cccc32)cc1 ; c1cc(-c2cc3ccccc3c3ccccc23)cc(N(c2ccc(-c3cc4ccccc4c4ccccc34)cc2)c2ccc(-c3cccc4c3oc3ccccc34)cc2)c1]

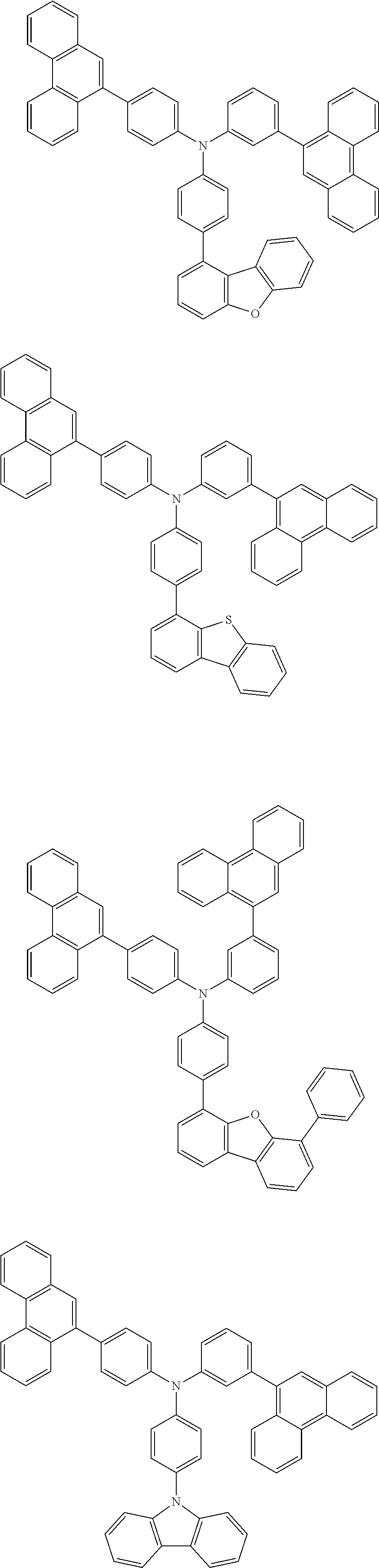 [SMILES: c1cc(-c2cc3ccccc3c3ccccc23)cc(N(c2ccc(-c3cc4ccccc4c4ccccc34)cc2)c2ccc(-c3cccc4oc5ccccc5c34)cc2)c1 ; c1cc(-c2cc3ccccc3c3ccccc23)cc(N(c2ccc(-c3cc4ccccc4c4ccccc34)cc2)c2ccc(-c3cccc4c3sc3ccccc34)cc2)c1 ; c1ccc(-c2cccc3c2oc2c(-c4ccc(N(c5ccc(-c6cc7ccccc7c7ccccc67)cc5)c5cccc(-c6cc7ccccc7c7ccccc67)c5)cc4)cccc23)cc1 ; c1cc(-c2cc3ccccc3c3ccccc23)cc(N(c2ccc(-c3cc4ccccc4c4ccccc34)cc2)c2ccc(-n3c4ccccc4c4ccccc43)cc2)c1]

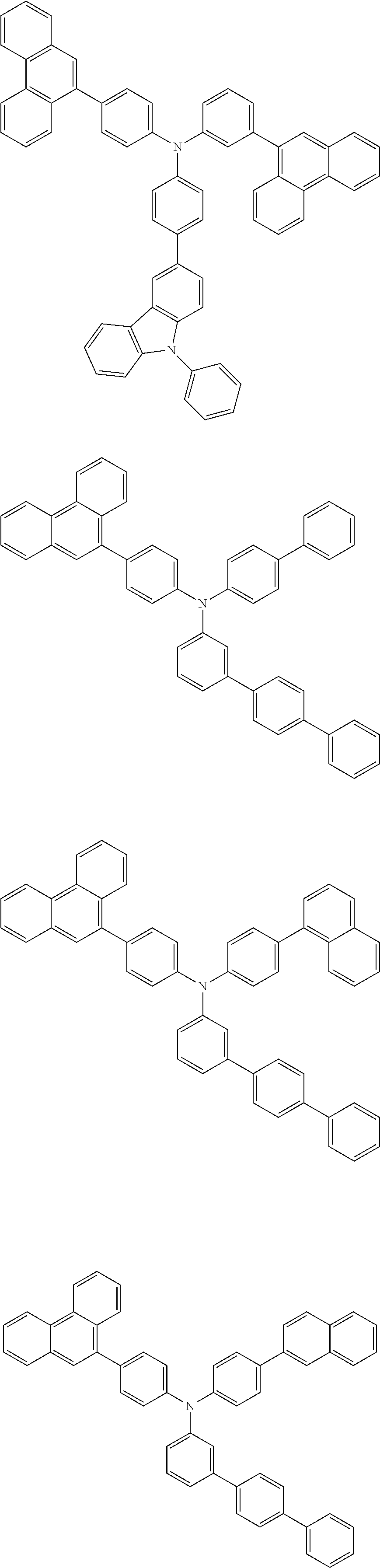 [SMILES: c1ccc(-n2c3ccccc3c3cc(-c4ccc(N(c5ccc(-c6cc7ccccc7c7ccccc67)cc5)c5cccc(-c6cc7ccccc7c7ccccc67)c5)cc4)ccc32)cc1 ; c1ccc(-c2ccc(-c3cccc(N(c4ccc(-c5ccccc5)cc4)c4ccc(-c5cc6ccccc6c6ccccc56)cc4)c3)cc2)cc1 ; c1ccc(-c2ccc(-c3cccc(N(c4ccc(-c5cccc6ccccc56)cc4)c4ccc(-c5cc6ccccc6c6ccccc56)cc4)c3)cc2)cc1 ; c1ccc(-c2ccc(-c3cccc(N(c4ccc(-c5ccc6ccccc6c5)cc4)c4ccc(-c5cc6ccccc6c6ccccc56)cc4)c3)cc2)cc1]

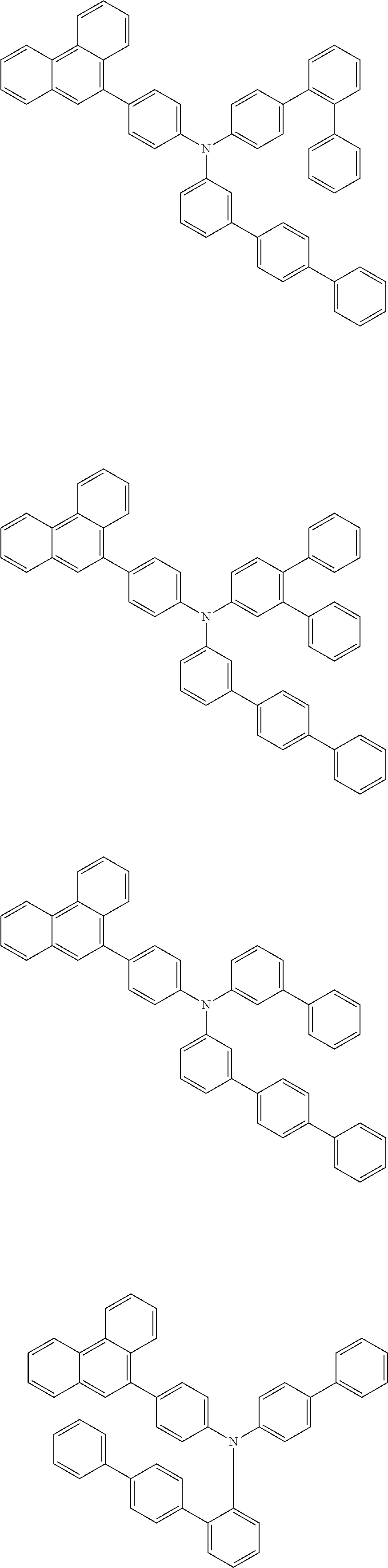 [SMILES: c1ccc(-c2ccc(-c3cccc(N(c4ccc(-c5ccccc5-c5ccccc5)cc4)c4ccc(-c5cc6ccccc6c6ccccc56)cc4)c3)cc2)cc1 ; c1ccc(-c2ccc(-c3cccc(N(c4ccc(-c5cc6ccccc6c6ccccc56)cc4)c4ccc(-c5ccccc5)c(-c5ccccc5)c4)c3)cc2)cc1 ; c1ccc(-c2ccc(-c3cccc(N(c4ccc(-c5cc6ccccc6c6ccccc56)cc4)c4cccc(-c5ccccc5)c4)c3)cc2)cc1 ; c1ccc(-c2ccc(-c3ccccc3N(c3ccc(-c4ccccc4)cc3)c3ccc(-c4cc5ccccc5c5ccccc45)cc3)cc2)cc1]

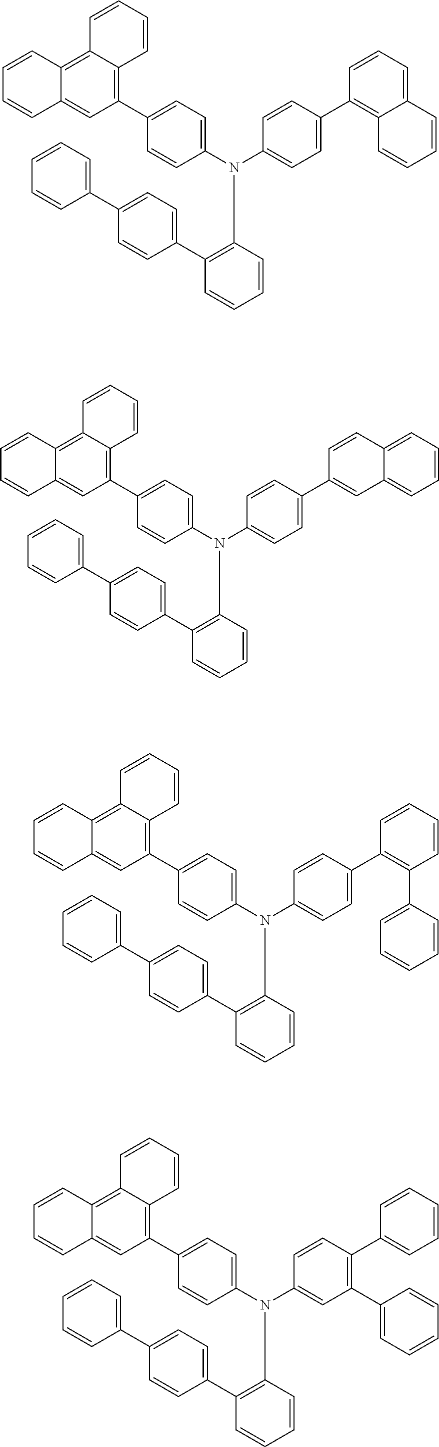 [SMILES: c1ccc(-c2ccc(-c3ccccc3N(c3ccc(-c4cccc5ccccc45)cc3)c3ccc(-c4cc5ccccc5c5ccccc45)cc3)cc2)cc1 ; c1ccc(-c2ccc(-c3ccccc3N(c3ccc(-c4ccc5ccccc5c4)cc3)c3ccc(-c4cc5ccccc5c5ccccc45)cc3)cc2)cc1 ; c1ccc(-c2ccc(-c3ccccc3N(c3ccc(-c4ccccc4-c4ccccc4)cc3)c3ccc(-c4cc5ccccc5c5ccccc45)cc3)cc2)cc1 ; c1ccc(-c2ccc(-c3ccccc3N(c3ccc(-c4cc5ccccc5c5ccccc45)cc3)c3ccc(-c4ccccc4)c(-c4ccccc4)c3)cc2)cc1]

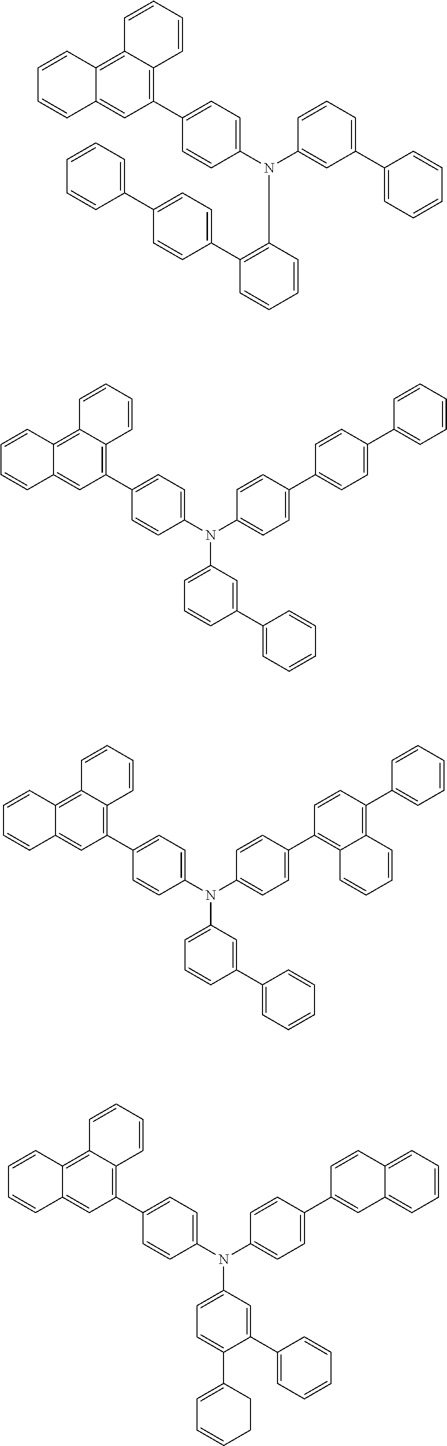 [SMILES: c1ccc(-c2ccc(-c3ccccc3N(c3ccc(-c4cc5ccccc5c5ccccc45)cc3)c3cccc(-c4ccccc4)c3)cc2)cc1 ; c1ccc(-c2ccc(-c3ccc(N(c4ccc(-c5cc6ccccc6c6ccccc56)cc4)c4cccc(-c5ccccc5)c4)cc3)cc2)cc1 ; c1ccc(-c2cccc(N(c3ccc(-c4ccc(-c5ccccc5)c5ccccc45)cc3)c3ccc(-c4cc5ccccc5c5ccccc45)cc3)c2)cc1 ; C1=CCCC(c2ccc(N(c3ccc(-c4ccc5ccccc5c4)cc3)c3ccc(-c4cc5ccccc5c5ccccc45)cc3)cc2-c2ccccc2)=C1]

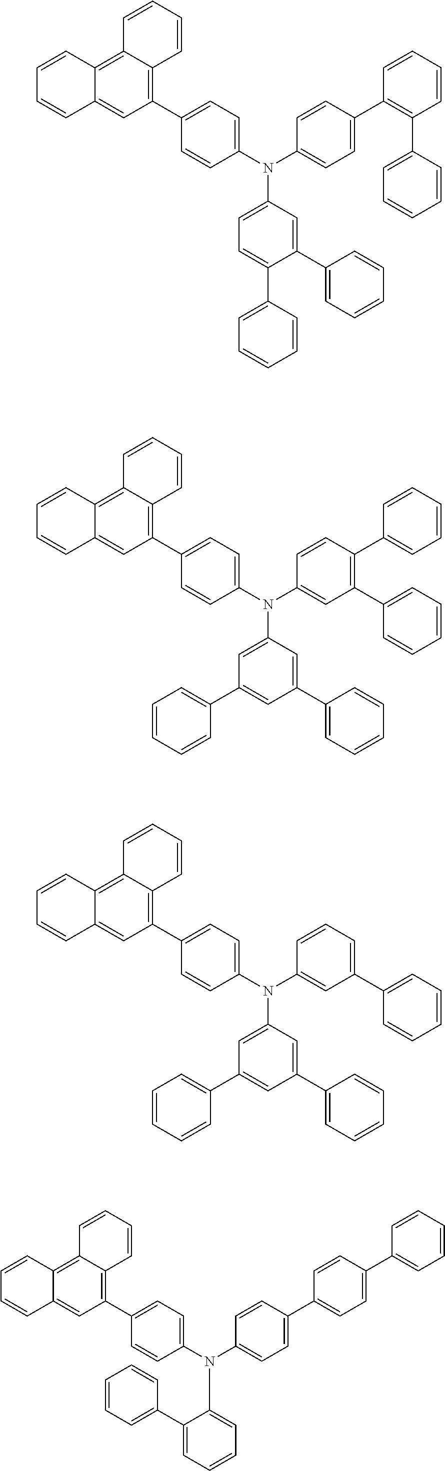 [SMILES: c1ccc(-c2ccccc2-c2ccc(N(c3ccc(-c4cc5ccccc5c5ccccc45)cc3)c3ccc(-c4ccccc4)c(-c4ccccc4)c3)cc2)cc1 ; c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2ccc(-c3ccc(N(c4ccc(-c5cc6ccccc6c6ccccc56)cc4)c4ccccc4-c4ccccc4)cc3)cc2)cc1]

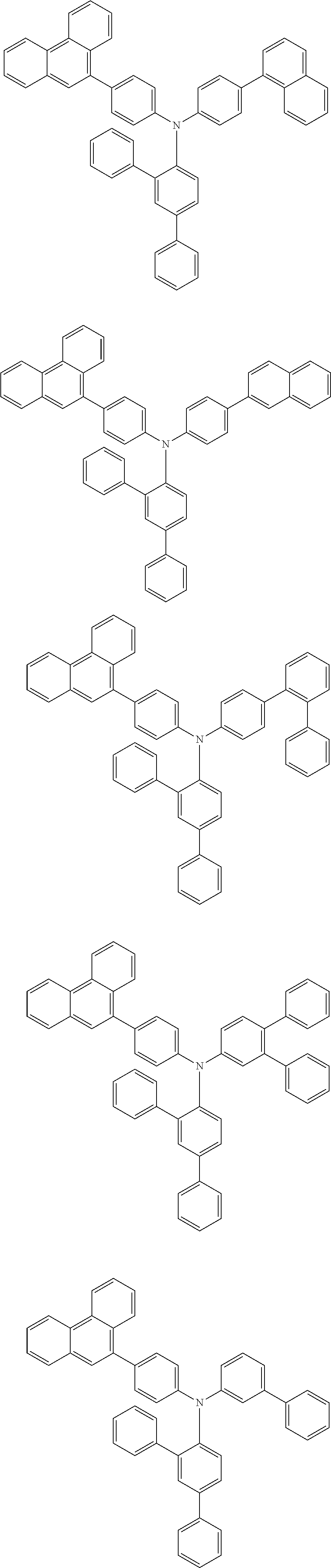 [SMILES: c1ccc(-c2ccc(N(c3ccc(-c4cccc5ccccc45)cc3)c3ccc(-c4cc5ccccc5c5ccccc45)cc3)c(-c3ccccc3)c2)cc1 ; c1ccc(-c2ccc(N(c3ccc(-c4ccc5ccccc5c4)cc3)c3ccc(-c4cc5ccccc5c5ccccc45)cc3)c(-c3ccccc3)c2)cc1 ; c1ccc(-c2ccc(N(c3ccc(-c4ccccc4-c4ccccc4)cc3)c3ccc(-c4cc5ccccc5c5ccccc45)cc3)c(-c3ccccc3)c2)cc1 ; c1ccc(-c2ccc(N(c3ccc(-c4cc5ccccc5c5ccccc45)cc3)c3ccc(-c4ccccc4)c(-c4ccccc4)c3)c(-c3ccccc3)c2)cc1 ; c1ccc(-c2cccc(N(c3ccc(-c4cc5ccccc5c5ccccc45)cc3)c3ccc(-c4ccccc4)cc3-c3ccccc3)c2)cc1]

Further provided herein is a method for preparing the compound represented by Chemical Formula 2 as shown in the following Reaction Scheme 2.

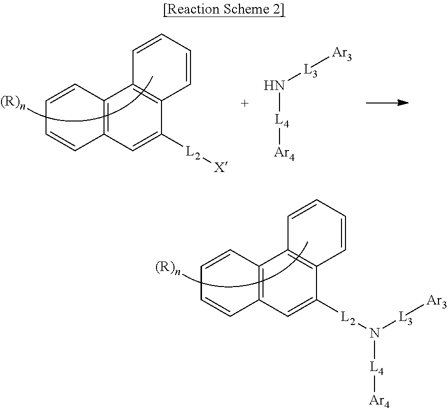

wherein in Reaction Scheme 2, the definition of the remaining substituents except for X′ are the same as defined above, and X′ is halogen, preferably bromo or chloro. The above reaction is an amine substitution reaction which is preferably carried out in the presence of a palladium catalyst and a base, and a reactive group for the amine substitution reaction can be modified as known in the art. The above preparation method may be further embodied in Preparation Examples described hereinafter.

Preferably, in the light emitting layer, the weight ratio of the compound represented by Chemical Formula 1 and the compound represented by Chemical Formula 2 is 10:90 to 90:10, more preferably 20:80 to 80:20, 30:70 to 70:30 or 40:60 to 60:40.

Meanwhile, the light emitting layer may further include a dopant in addition to the host. The dopant material is not particularly limited as long as it is a material used for the organic light emitting device. As an example, an aromatic amine derivative, a styrylamine compound, a boron complex, a fluoranthene compound, a metal complex, and the like can be mentioned. Specific examples of the aromatic amine derivatives include substituted or unsubstituted fused aromatic ring derivatives having an arylamino group, examples thereof include pyrene, anthracene, chrysene, and periflanthene having the arylamino group, and the like. The styrylamine compound is a compound where at least one arylvinyl group is substituted in substituted or unsubstituted arylamine, wherein one or two or more substituent groups selected from the group consisting of an aryl group, a silyl group, an alkyl group, a cycloalkyl group and an arylamino group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

Electron Transport Layer

The organic light emitting device according to the present disclosure may include an electron transport layer on the light emitting layer, if necessary.

The electron transport layer is a layer that receives electrons from the electron injection layer formed on the cathode and anode and transports the electrons to the light emitting layer, and that suppress the transfer of holes from the light emitting layer, and an electron transport material is suitably a material which may receive electrons well from a cathode and transfer the electrons to a light emitting layer, and has a large mobility for electrons.

Specific examples of the electron transport material include: an Al complex of 8-hydroxyquinoline; a complex including Alq₃; an organic radical compound; a hydroxyflavone-metal complex, and the like, but are not limited thereto. The electron transport layer may be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

Electron Injection Layer

The organic light emitting device according to the present disclosure may further include an electron injection layer on the light emitting layer (or on an electron transport layer when the electron transport layer is present), if necessary.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the materials that can be used as the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Organic Light Emitting Device

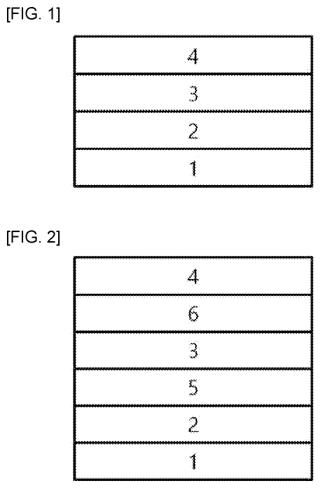

The structure of the organic light emitting device according to the present disclosure is illustrated in FIGS. 1 and 2. FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole transport layer 5, a light emitting layer 3, an electron transport layer 6, and a cathode 4.

The organic light emitting device according to the present disclosure can be manufactured by sequentially stacking the above-described structures. In this case, the organic light emitting device may be manufactured by depositing a metal, metal oxides having conductivity, or an alloy thereof on the substrate by using a PVD (physical vapor deposition) method such as a sputtering method or an e-beam evaporation method to form the anode, forming the respective layers described above thereon, and then depositing a material that can be used as the cathode thereon. In addition to such a method, the organic light emitting device can be manufactured by sequentially depositing from the cathode material to the anode material on a substrate in the reverse order of the above-mentioned configuration (WO 2003/012890). Further, the light emitting layer may be formed by subjecting hosts and dopants to a vacuum deposition method and a solution coating method. Herein, the solution coating method means a spin coating, a dip coating, a doctor blading, an inkjet printing, a screen printing, a spray method, a roll coating, or the like, but is not limited thereto.

On the other hand, the organic light emitting device according to the present disclosure may be a front side emission type, a back side emission type, or a double side emission type according to the used material.

The preparation of the organic light emitting device including the compound represented by Chemical Formula 1 and the compound represented by Chemical Formula 2 will be described in detail in the following examples. However, these examples are presented for illustrative purposes only, and are not intended to limit the scope of the present disclosure.

### PREPARATION EXAMPLE

### Preparation Example 1-1

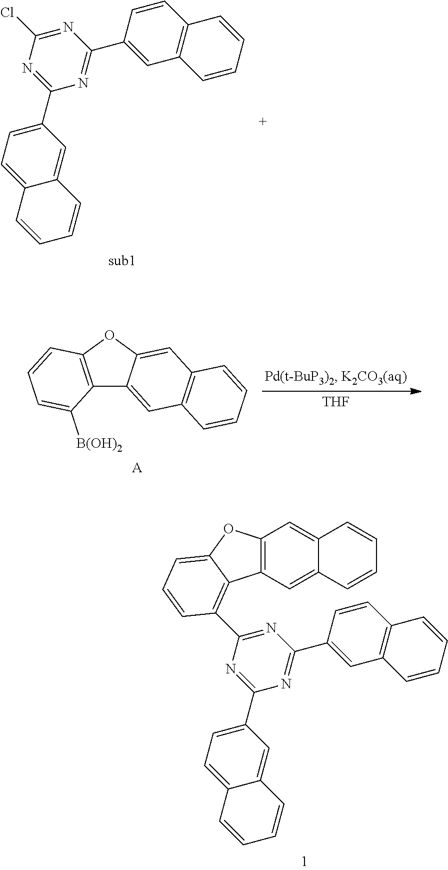

Compound sub1 (15 g, 40.8 mmol) and Compound A (11.8 g, 44.9 mmol) were added to THF (300 ml) under a nitrogen atmosphere, and the mixture was stirred and refluxed. Then, potassium carbonate (16.9 g, 122.3 mmol) was dissolved in water (51 ml), added thereto, and the mixture was sufficiently stirred and then bis(tri-tert-butylphosphine)palladium (0) (0.2 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 1 (14.6 g). (Yield: 65%, MS: [M+H]⁺=550)

### Preparation Example 1-2

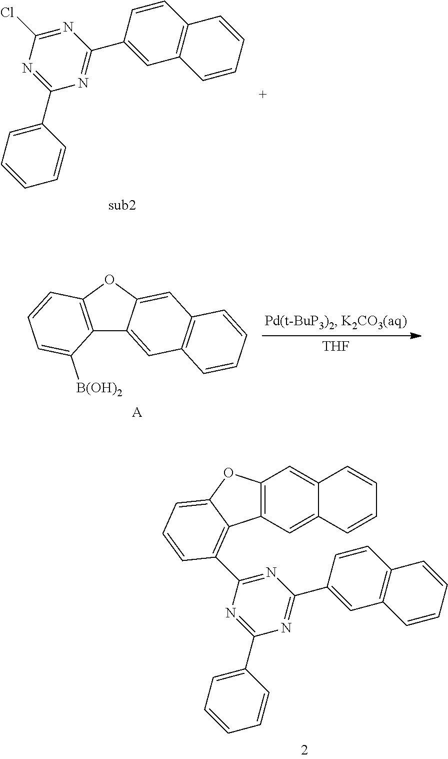

Compound sub2 (15 g, 47.2 mmol) and Compound A (13.6 g, 51.9 mmol) were added to THF (300 ml) under a nitrogen atmosphere, and the mixture was stirred and refluxed. Then, potassium carbonate (19.6 g, 141.6 mmol) was dissolved in water (59 ml), added thereto, and the mixture was sufficiently stirred and then bis(tri-tert-butylphosphine)palladium (0) (0.2 g, 0.5 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 2 (14.4 g). (Yield: 61%, MS: [M+H]⁺=500)

### Preparation Example 1-3

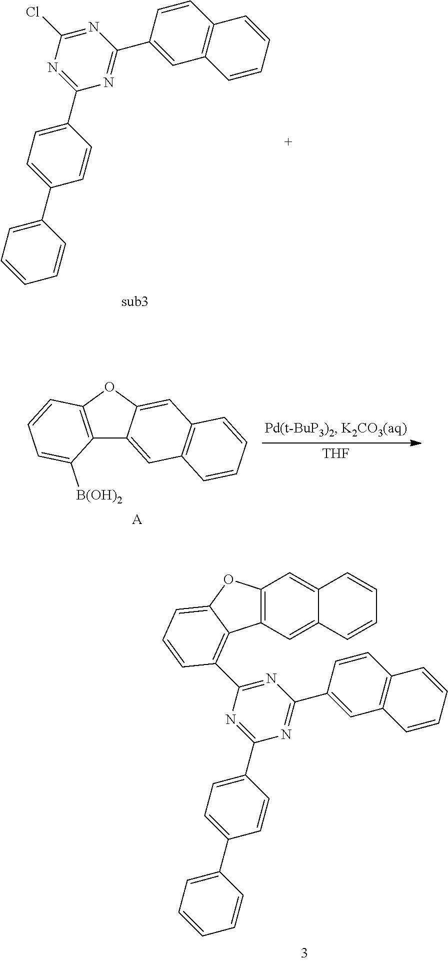

Compound sub3 (15 g, 38.1 mmol) and Compound A (11 g, 41.9 mmol) were added to THF (300 ml) under a nitrogen atmosphere, and the mixture was stirred and refluxed. Then, potassium carbonate (15.8 g, 114.3 mmol) was dissolved in water (47 ml), added thereto, and the mixture was sufficiently stirred and then bis(tri-tert-butylphosphine)palladium (0) (0.2 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 3 (13.4 g). (Yield: 61%, MS: [M+H]⁺=576)

### Preparation Example 1-4

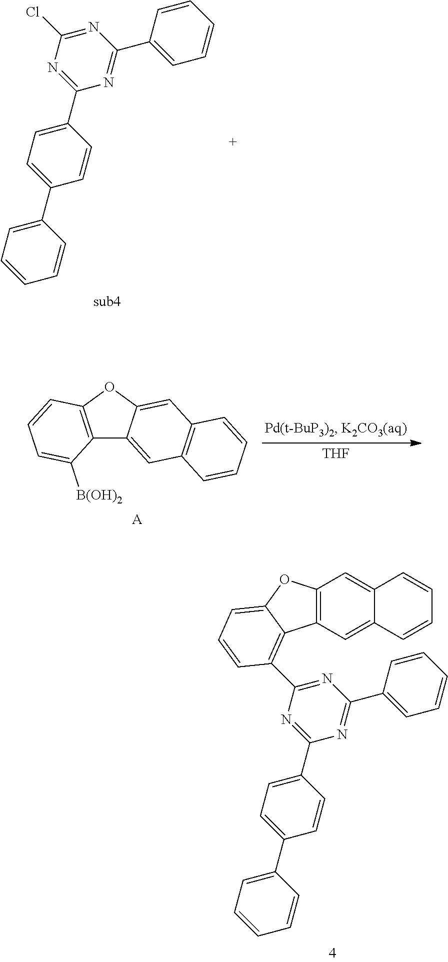

Compound sub4 (15 g, 43.6 mmol) and Compound A (12.6 g, 48 mmol) were added to THF (300 ml) under a nitrogen atmosphere, and the mixture was stirred and refluxed. Then, potassium carbonate (18.1 g, 130.9 mmol) was dissolved in water (54 ml), added thereto, and the mixture was sufficiently stirred and then bis(tri-tert-butylphosphine)palladium (0) (0.2 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 4 (18.3 g). (Yield: 80%, MS: [M+H]⁺=526)

### Preparation Example 1-5

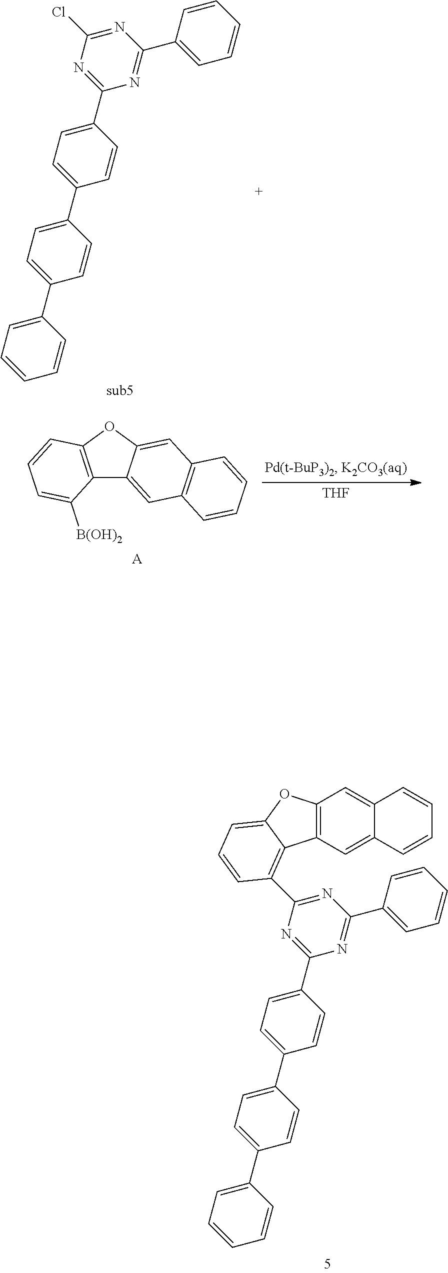

Compound sub5 (15 g, 35.7 mmol) and Compound A (10.3 g, 39.3 mmol) were added to THF (300 ml) under a nitrogen atmosphere, and the mixture was stirred and refluxed. Then, potassium carbonate (14.8 g, 107.2 mmol) was dissolved in water (44 ml), added thereto, and the mixture was sufficiently stirred and then bis(tri-tert-butylphosphine)palladium (0) (0.2 g, 0.4 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 5 (15.2 g). (Yield: 71%, MS: [M+H]⁺=602)

### Preparation Example 1-6

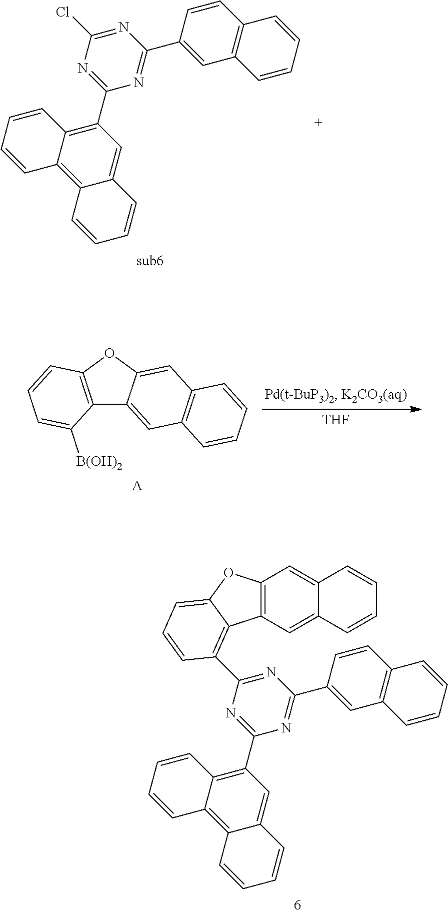

Compound sub6 (15 g, 35.9 mmol) and Compound A (10.3 g, 39.5 mmol) were added to THF (300 ml) under a nitrogen atmosphere, and the mixture was stirred and refluxed. Then, potassium carbonate (14.9 g, 107.7 mmol) was dissolved in water (45 ml), added thereto, and the mixture was sufficiently stirred and then bis(tri-tert-butylphosphine)palladium (0) (0.2 g. 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 6 (13.1 g). (Yield: 61%, MS: [M+H]⁺=600)

### Preparation Example 1-7

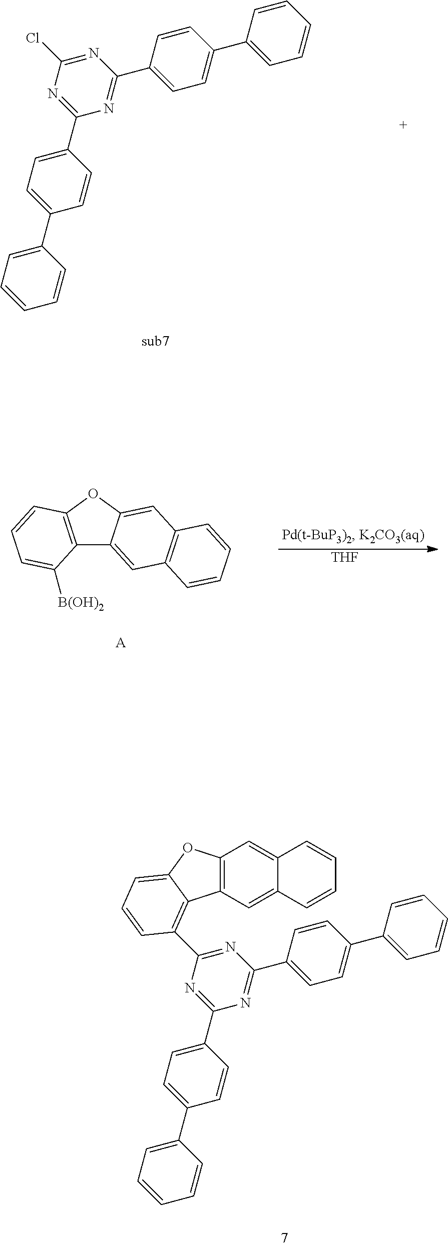

Compound sub7 (15 g, 35.7 mmol) and Compound A (10.3 g, 39.3 mmol) were added to THF (300 ml) under a nitrogen atmosphere, and the mixture was stirred and refluxed. Then, potassium carbonate (14.8 g, 107.2 mmol) was dissolved in water (44 ml), added thereto, and the mixture was sufficiently stirred and then bis(tri-tert-butylphosphine)palladium (0) (0.2 g, 0.4 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 7 (14.2 g). (Yield: 66%, MS: [M+H]⁺=602)

### Preparation Example 1-8

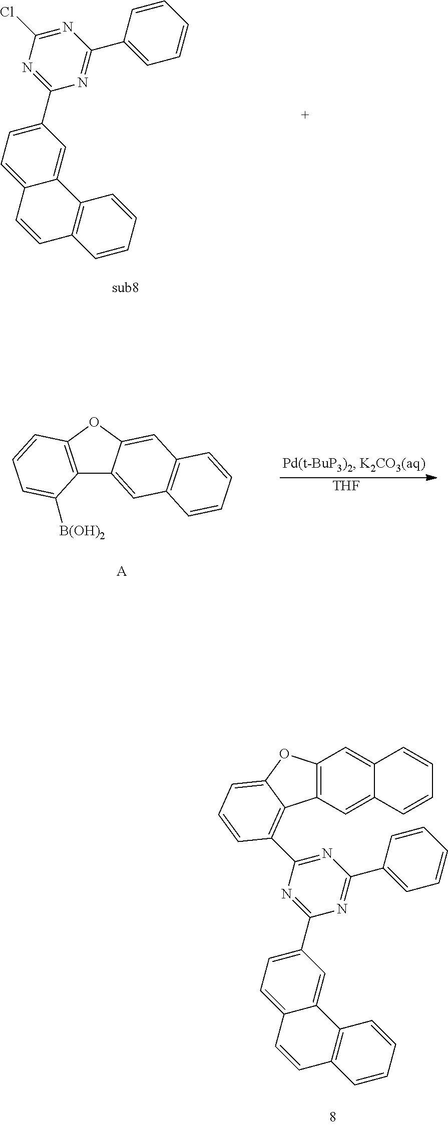

Compound sub8 (15 g, 40.8 mmol) and Compound A (11.8 g, 44.9 mmol) were added to THF (300 ml) under a nitrogen atmosphere, and the mixture was stirred and refluxed. Then, potassium carbonate (16.9 g, 122.3 mmol) was dissolved in water (51 ml), added thereto, and the mixture was sufficiently stirred and then bis(tri-tert-butylphosphine)palladium (0) (0.2 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 8 (13.4 g). (Yield: 60%, MS: [M+H]⁺=550)

### Preparation Example 1-9

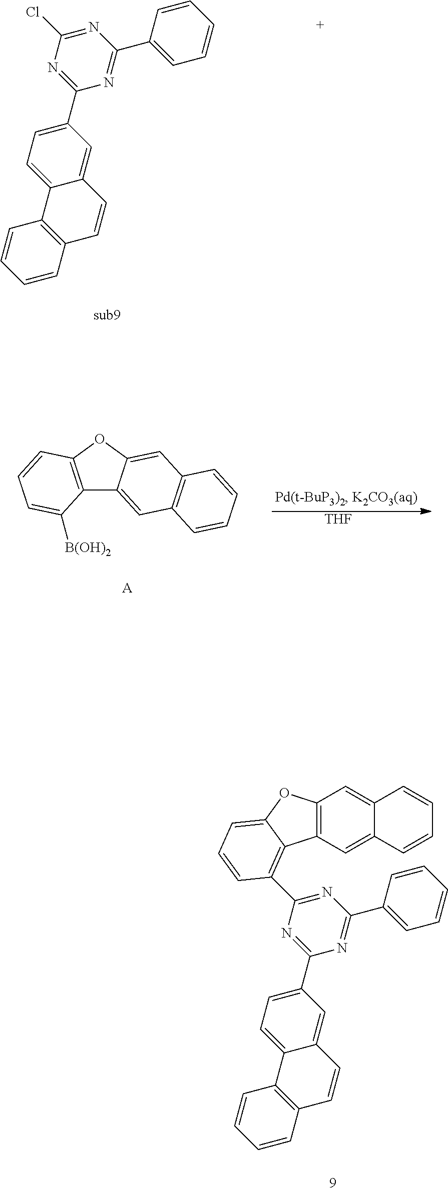

Compound sub9 (15 g, 40.8 mmol) and Compound A (11.8 g, 44.9 mmol) were added to THF (300 ml) under a nitrogen atmosphere, and the mixture was stirred and refluxed. Then, potassium carbonate (16.9 g, 122.3 mmol) was dissolved in water (51 ml), added thereto, and the mixture was sufficiently stirred and then bis(tri-tert-butylphosphine)palladium (0) (0.2 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 9 (14.1 g). (Yield: 63%, MS: [M+H]⁺=550)

### Preparation Example 1-10

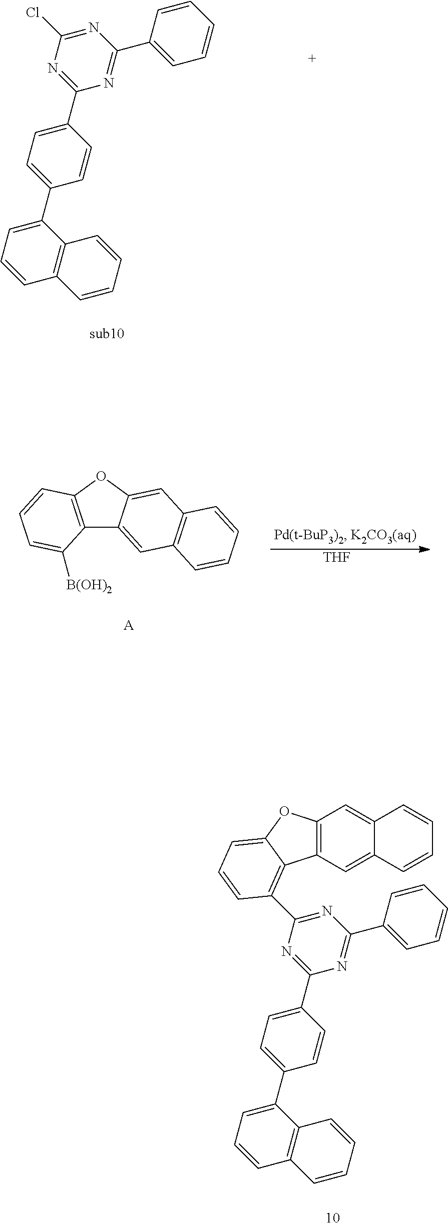

Compound sub10 (15 g, 38.1 mmol) and Compound A (11 g, 41.9 mmol) were added to THF (300 ml) under a nitrogen atmosphere, and the mixture was stirred and refluxed. Then, potassium carbonate (15.8 g, 114.3 mmol) was dissolved in water (47 ml), added thereto, and the mixture was sufficiently stirred and then bis(tri-tert-butylphosphine)palladium (0) (0.2 g, 0.4 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 10 (15.8 g). (Yield: 72%, MS: [M+H]⁺=576)

### Preparation Example 1-11

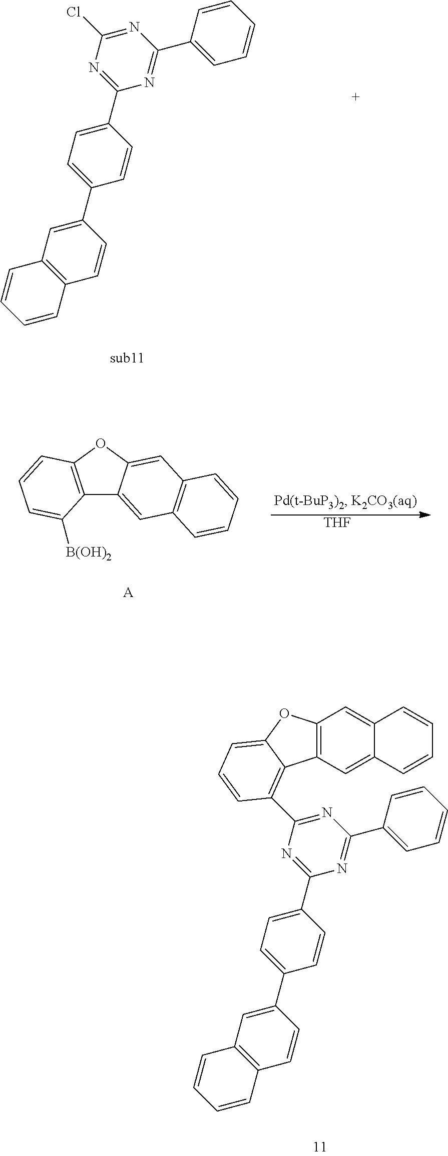

Compound sub11 (15 g, 38.1 mmol) and Compound A (11 g, 41.9 mmol) were added to THF (300 ml) under a nitrogen atmosphere, and the mixture was stirred and refluxed. Then, potassium carbonate (15.8 g, 114.3 mmol) was dissolved in water (47 ml), added thereto, and the mixture was sufficiently stirred and then bis(tri-tert-butylphosphine)palladium (0) (0.2 g, 0.4 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 11 (16.6 g). (Yield: 76%, MS: [M+H]⁺=576)

### Preparation Example 1-12

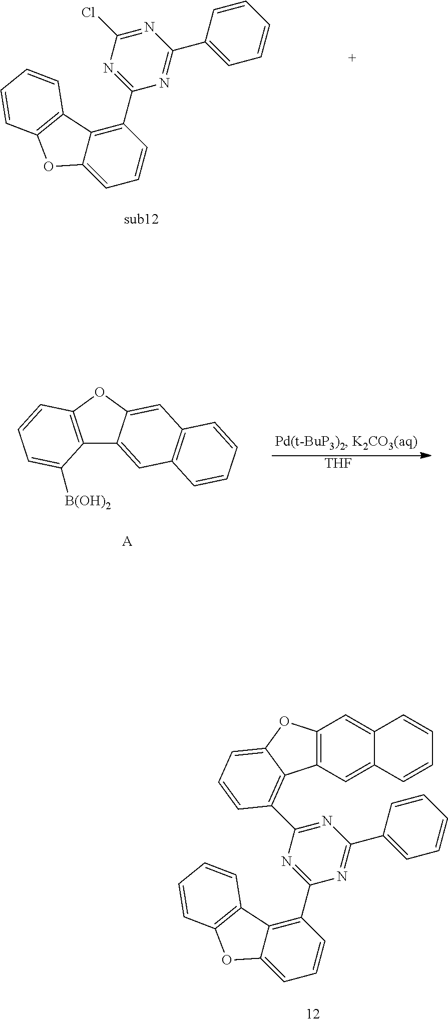

Compound sub12 (15 g, 41.9 mmol) and Compound A (12.1 g, 46.1 mmol) were added to THF (300 ml) under a nitrogen atmosphere, and the mixture was stirred and refluxed. Then, potassium carbonate (17.4 g, 125.8 mmol) was dissolved in water (52 ml), added thereto, and the mixture was sufficiently stirred and then bis(tri-tert-butylphosphine)palladium (0) (0.2 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 12 (13.8 g). (Yield: 61%, MS: [M+H]⁺=540)

### Preparation Example 1-13

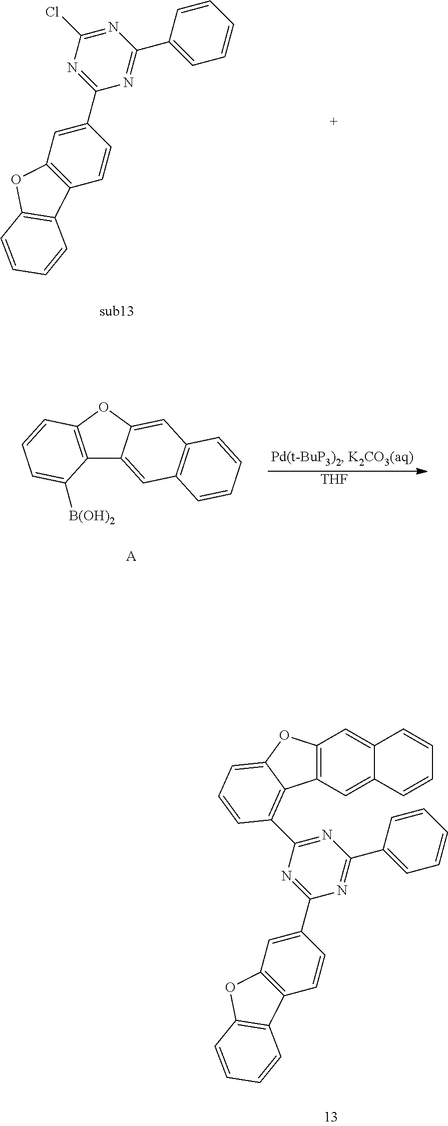

Compound sub13 (15 g, 41.9 mmol) and Compound A (12.1 g, 46.1 mmol) were added to THF (300 ml) under a nitrogen atmosphere, and the mixture was stirred and refluxed. Then, potassium carbonate (17.4 g, 125.8 mmol) was dissolved in water (52 ml), added thereto, and the mixture was sufficiently stirred and then bis(tri-tert-butylphosphine)palladium (0) (0.2 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 13 (15.4 g). (Yield: 68%, MS: [M+H]⁺=540)

### Preparation Example 1-14

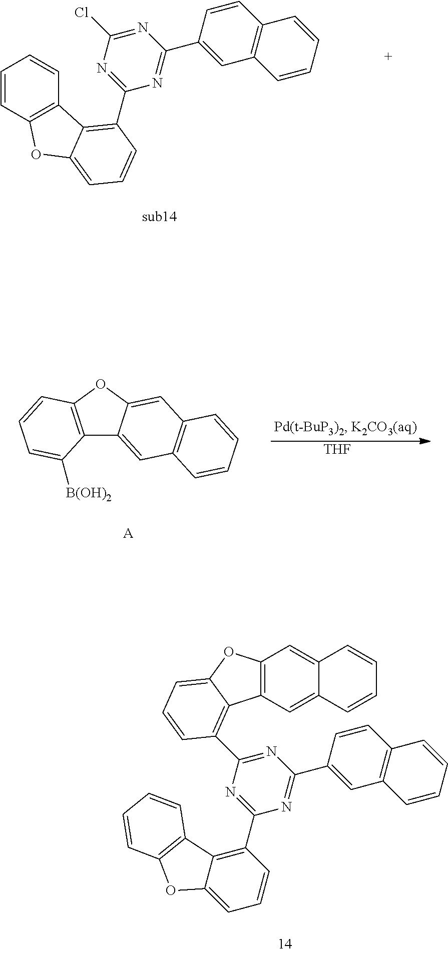

Compound sub14 (15 g, 36.8 mmol) and Compound A (10.6 g, 40.5 mmol) were added to THF (300 ml) under a nitrogen atmosphere, and the mixture was stirred and refluxed. Then, potassium carbonate (15.2 g, 110.3 mmol) was dissolved in water (46 ml), added thereto, and the mixture was sufficiently stirred and then bis(tri-tert-butylphosphine)palladium (0) (0.2 g, 0.4 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 14 (16.3 g). (Yield: 75%, MS: [M+H]⁺=590)

### Preparation Example 1-15

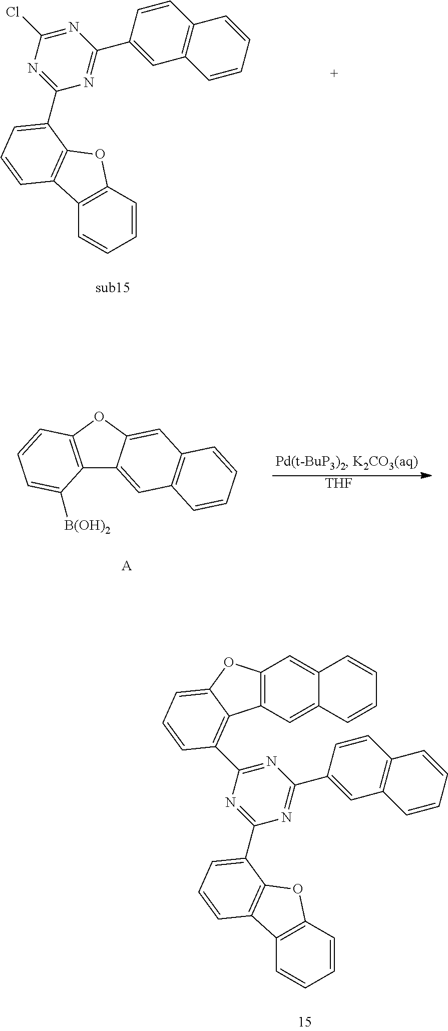

Compound sub15 (15 g, 36.8 mmol) and Compound A (10.6 g, 40.5 mmol) were added to THF (300 ml) under a nitrogen atmosphere, and the mixture was stirred and refluxed. Then, potassium carbonate (15.2 g, 110.3 mmol) was dissolved in water (46 ml), added thereto, and the mixture was sufficiently stirred and then bis(tri-tert-butylphosphine)palladium (0) (0.2 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 15 (15.2 g). (Yield: 70%, MS: [M+H]⁺=590)

### Preparation Example 1-16

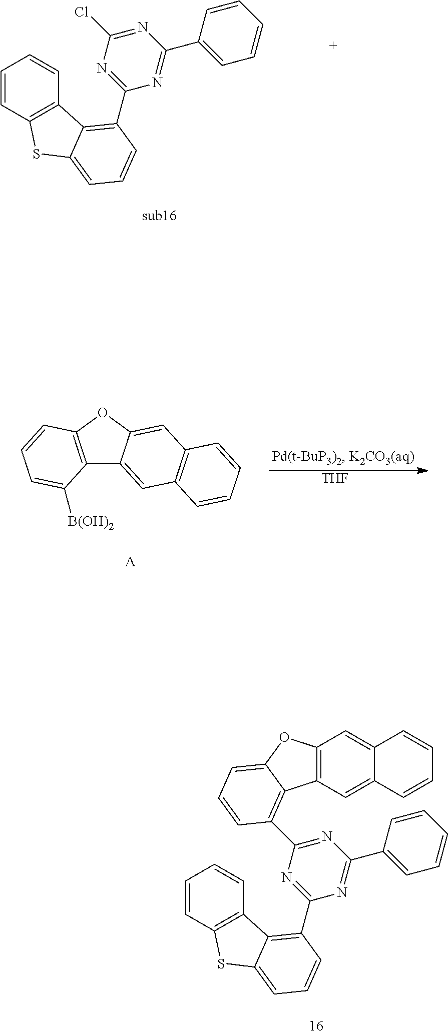

Compound sub16 (15 g, 40.1 mmol) and Compound A (11.6 g, 44.1 mmol) were added to THF (300 ml) under a nitrogen atmosphere, and the mixture was stirred and refluxed. Then, potassium carbonate (16.6 g, 120.4 mmol) was dissolved in water (50 ml), added thereto, and the mixture was sufficiently stirred and then bis(tri-tert-butylphosphine)palladium (0) (9.2 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 16 (13.8 g). (Yield: 62%, MS: [M+H]⁺=556)

### Preparation Example 1-17

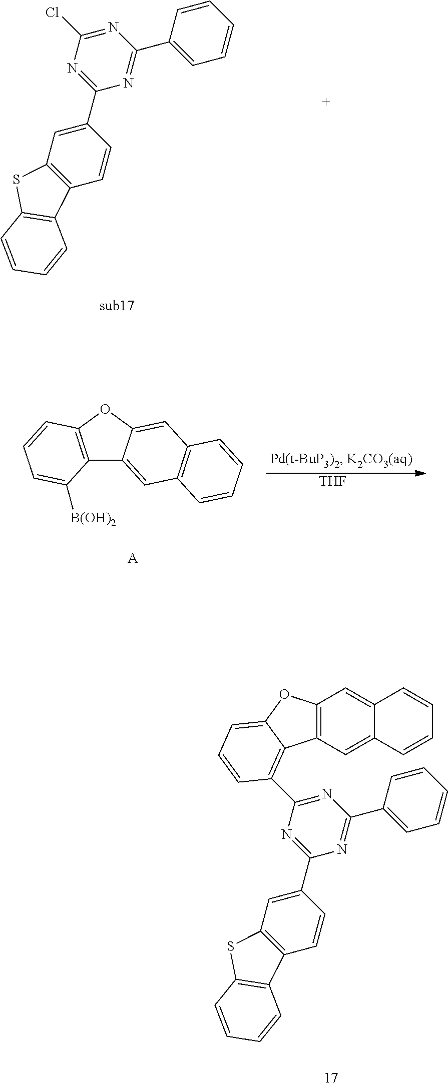

Compound sub17 (15 g, 40.1 mmol) and Compound A (11.6 g, 44.1 mmol) were added to THF (300 ml) under a nitrogen atmosphere, and the mixture was stirred and refluxed. Then, potassium carbonate (16.6 g, 120.4 mmol) was dissolved in water (50 ml), added thereto, and the mixture was sufficiently stirred and then bis(tri-tert-butylphosphine)palladium (0) (0.2 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 17 (15.1 g). (Yield: 68%, MS: [M+H]⁺=556)

### Preparation Example 1-18

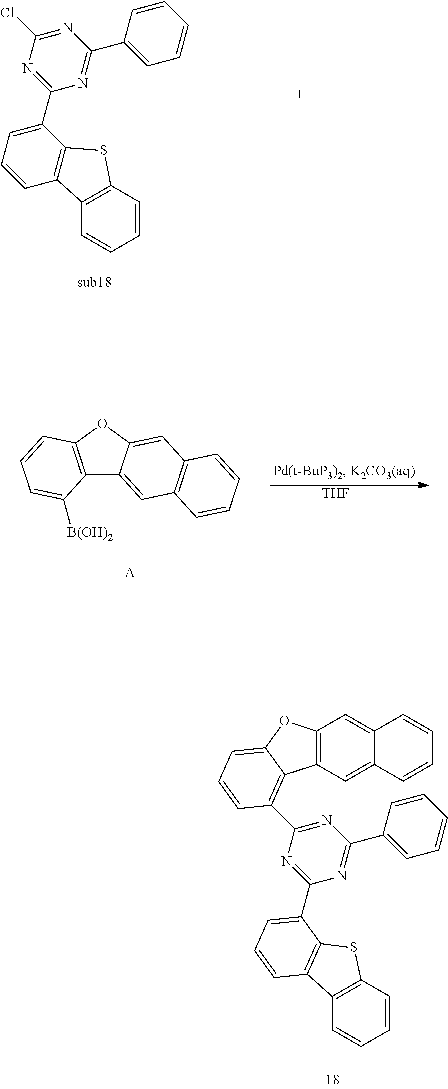

Compound sub18 (15 g, 40.1 mmol) and Compound A (11.6 g, 44.1 mmol) were added to THF (300 ml) under a nitrogen atmosphere, and the mixture was stirred and refluxed. Then, potassium carbonate (16.6 g, 120.4 mmol) was dissolved in water (50 ml), added thereto, and the mixture was sufficiently stirred and then bis(tri-tert-butylphosphine)palladium (0) (0.2 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 18 (17.8 g). (Yield: 80%, MS: [M+H]⁺=556)

### Preparation Example 1-19

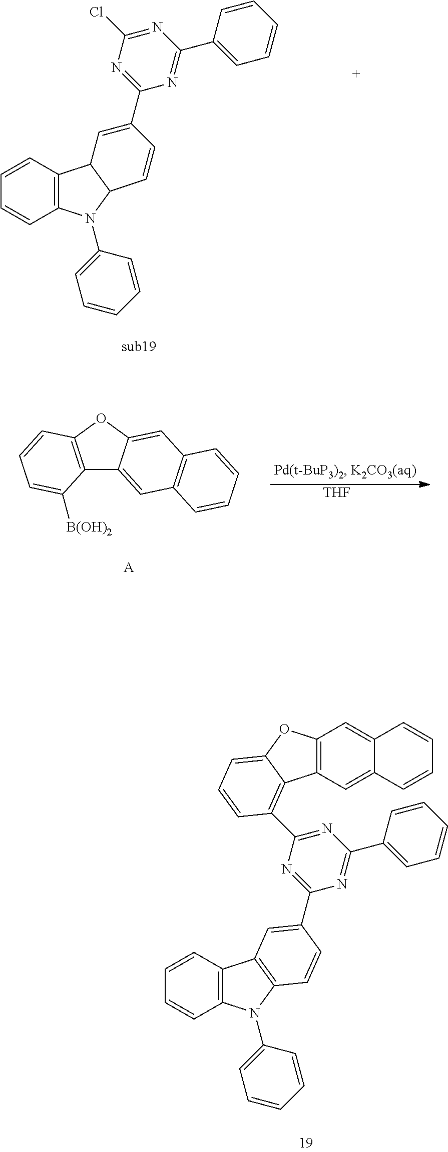

Compound sub19 (15 g, 34.6 mmol) and Compound A (10 g, 38.1 mmol) were added to THF (300 ml) under a nitrogen atmosphere, and the mixture was stirred and refluxed. Then, potassium carbonate (14.4 g, 103.9 mmol) was dissolved in water (43 ml), added thereto, and the mixture was sufficiently stirred and then bis(tri-tert-butylphosphine)palladium (0) (0.2 g, 0.3 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 19 (15.5 g). (Yield: 73%, MS: [M+H]⁺=615)

### Preparation Example 1-20

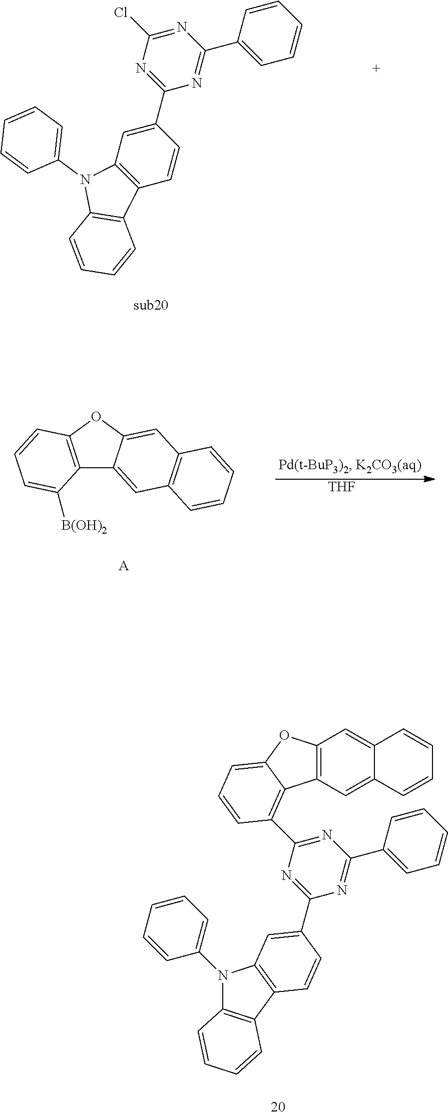

Compound sub20 (15 g, 34.6 mmol) and Compound A (10 g, 38.1 mmol) were added to THF (300 ml) under a nitrogen atmosphere, and the mixture was stirred and refluxed. Then, potassium carbonate (14.4 g, 103.9 mmol) was dissolved in water (43 ml), added thereto, and the mixture was sufficiently stirred and then bis(tri-tert-butylphosphine)palladium (0) (0.2 g, 0.3 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 20 (17 g). (Yield: 80%, MS: [M+H]⁺=615)

### Preparation Example 1-21

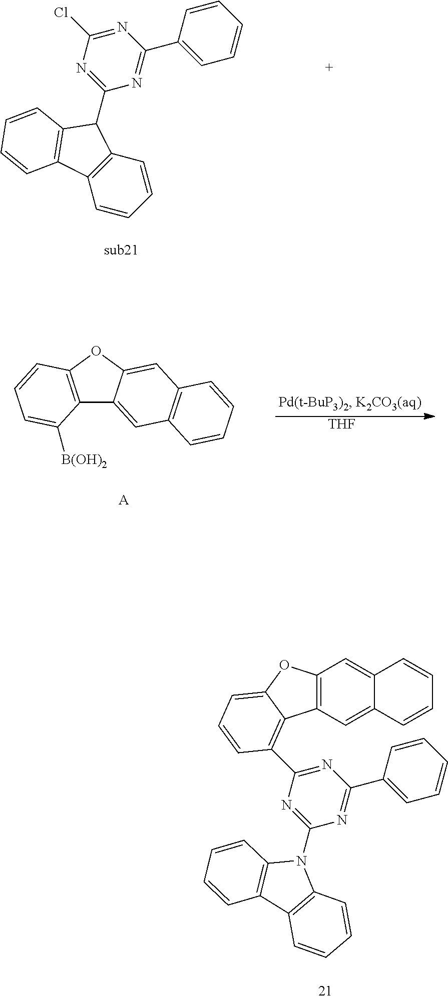

Compound sub21 (15 g, 42 mmol) and Compound A (12.1 g, 46.2 mmol) were added to THF (300 ml) under a nitrogen atmosphere, and the mixture was stirred and refluxed. Then, potassium carbonate (17.4 g, 126.1 mmol) was dissolved in water (52 ml), added thereto, and the mixture was sufficiently stirred and then bis(tri-tert-butylphosphine)palladium (0) (0.2 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 21 (14.5 g). (Yield: 64%, MS: [M+H]⁺=539)

### Preparation Example 1-22

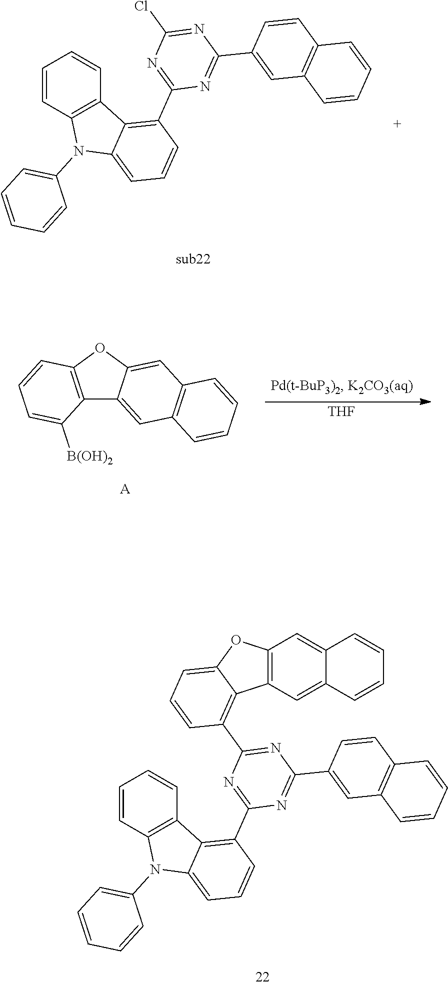

Compound sub22 (15 g, 31.1 mmol) and Compound A (9 g, 34.2 mmol) were added to THF (300 ml) under a nitrogen atmosphere, and the mixture was stirred and refluxed. Then, potassium carbonate (12.9 g, 93.2 mmol) was dissolved in water (39 ml), added thereto, and the mixture was sufficiently stirred and then bis(tri-tert-butylphosphine)palladium (0) (0.2 g, 0.3 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 22 (12.4 g). (Yield: 60%, MS: [M+H]⁺=665)

### Preparation Example 1-23

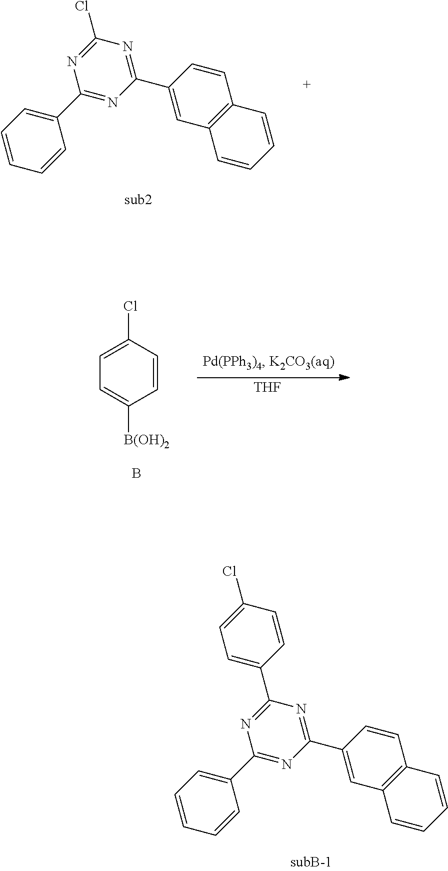

Compound sub2 (15 g, 47.2 mmol) and Compound B (7.4 g, 47.2 mmol) were added to THF (300 ml) under a nitrogen atmosphere, and the mixture was stirred and refluxed. Then, potassium carbonate (19.6 g, 141.6 mmol) was dissolved in water (59 ml), added thereto, and the mixture was sufficiently stirred and then tetrakis(triphenylphosphine)palladium (0) (0.5 g, 0.5 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subB-1 (13.9 g). (Yield: 75%, MS: [M+H]⁺=394)

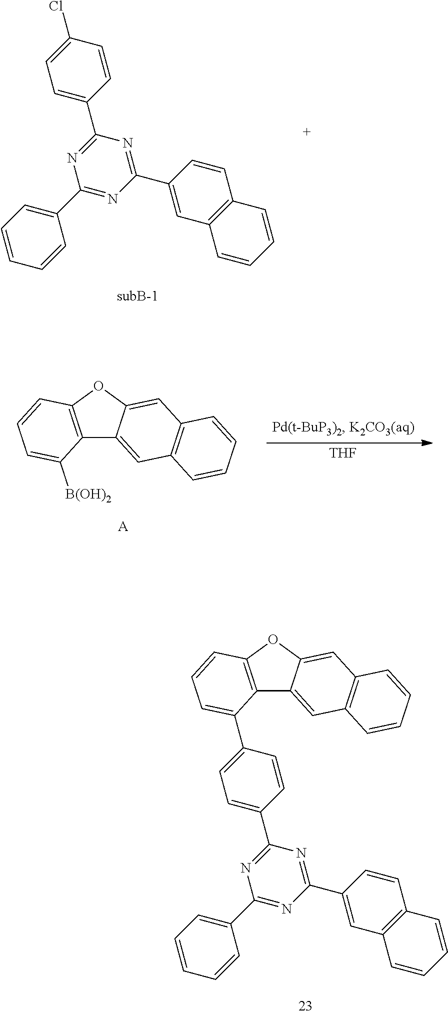

Compound subB-1 (15 g, 38.1 mmol) and Compound A (11 g, 41.9 mmol) were added to THF (300 ml) under a nitrogen atmosphere, and the mixture was stirred and refluxed. Then, potassium carbonate (15.8 g, 114.3 mmol) was dissolved in water (47 ml), added thereto, and the mixture was sufficiently stirred and then bis(tri-tert-butylphosphine)palladium (0) (0.2 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 23 (15.3 g). (Yield: 70%, MS: [M+H]⁺=576)

### Preparation Example 1-24

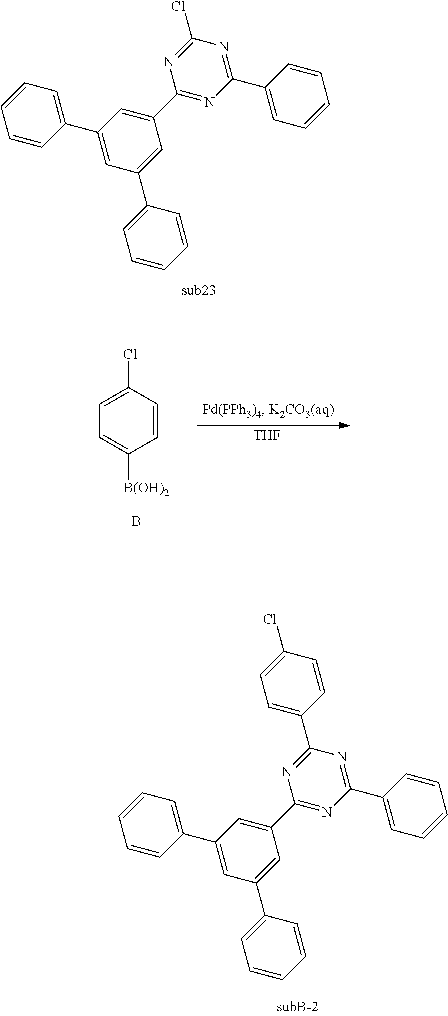

Compound sub23 (15 g, 35.7 mmol) and Compound B (5.6 g, 35.7 mmol) were added to THF (300 ml) under a nitrogen atmosphere, and the mixture was stirred and refluxed. Then, potassium carbonate (14.8 g, 107.2 mmol) was dissolved in water (44 ml), added thereto, and the mixture was sufficiently stirred and then tetrakis(triphenylphosphine)palladium (0) (0.4 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subB-2 (12 g). (Yield: 68%, MS: [M+H]⁺=496)

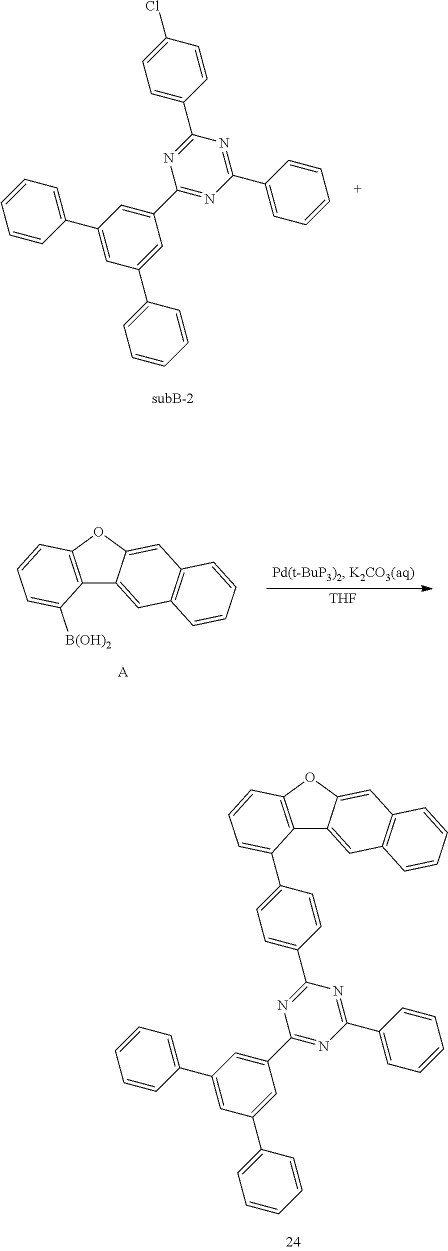

Compound subB-2 (15 g, 30.2 mmol) and Compound A (8.7 g, 33.3 mmol) were added to THF (300 ml) under a nitrogen atmosphere, and the mixture was stirred and refluxed. Then, potassium carbonate (12.5 g, 90.7 mmol) was dissolved in water (38 ml), added thereto, and the mixture was sufficiently stirred and then bis(tri-tert-butylphosphine)palladium (0) (0.2 g, 0.3 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 24 (13.1 g). (Yield: 64%, MS: [M+H]⁺=678)

### Preparation Example 1-25

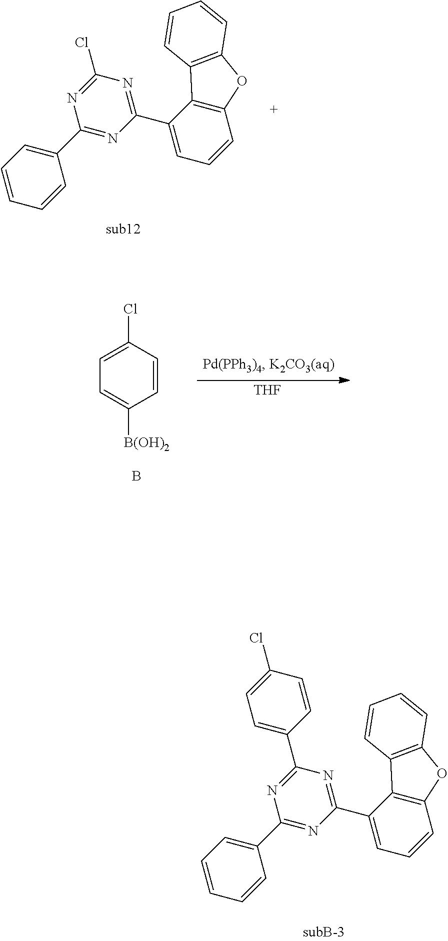

Compound sub12 (15 g, 41.9 mmol) and Compound B (6.6 g, 41.9 mmol) were added to THF (300 ml) under a nitrogen atmosphere, and the mixture was stirred and refluxed. Then, potassium carbonate (17.4 g, 125.8 mmol) was dissolved in water (52 ml), added thereto, and the mixture was sufficiently stirred and then tetrakis(triphenylphosphine)palladium(0) (0.5 g, 0.4 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subB-3 (12.9 g). (Yield: 71%, MS: [M+H]⁺=434)

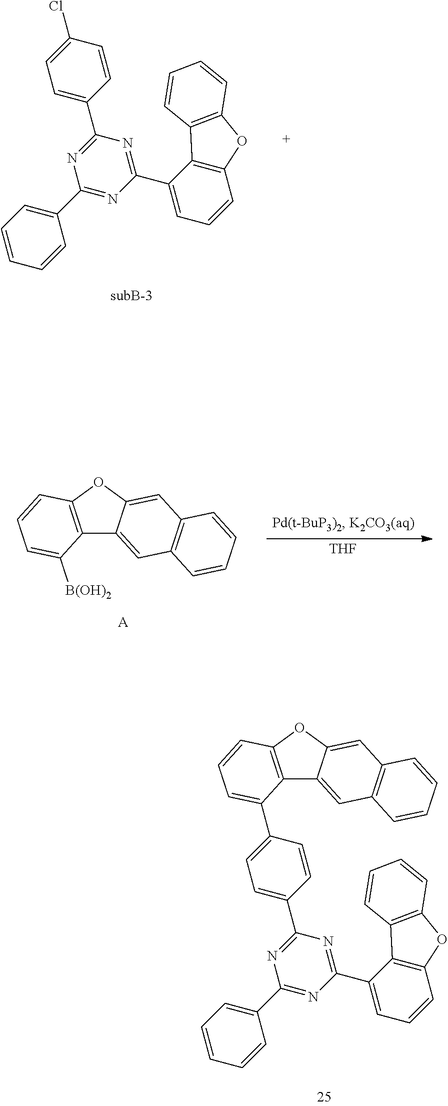

Compound subB-3 (15 g, 34.6 mmol) and Compound A (10 g, 38 mmol) were added to THF (300 ml) under a nitrogen atmosphere, and the mixture was stirred and refluxed. Then, potassium carbonate (14.3 g, 103.7 mmol) was dissolved in water (43 ml), added thereto, and the mixture was sufficiently stirred and then bis(tri-tert-butylphosphine)palladium(0) (0.2 g, 0.3 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 25 (17 g). (Yield: 80%, MS: [M+H]⁺=616)

### Preparation Example 1-26

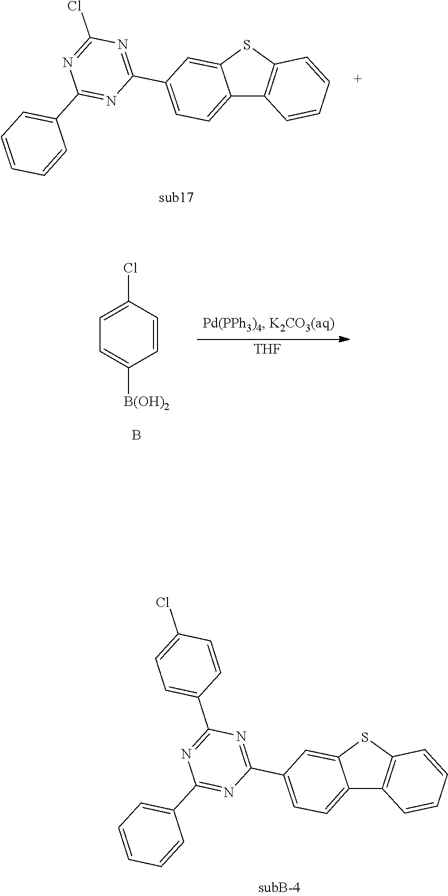

Compound sub17 (15 g, 40.1 mmol) and Compound B (6.3 g, 40.1 mmol) were added to THF (300 ml) under a nitrogen atmosphere, and the mixture was stirred and refluxed. Then, potassium carbonate (16.6 g, 120.4 mmol) was dissolved in water (50 ml), added thereto, and the mixture was sufficiently stirred and then tetrakis(triphenylphosphine)palladium(0) (0.5 g, 0.4 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subB-4 (12.1 g). (Yield: 67%, MS: [M+H]⁺=450)

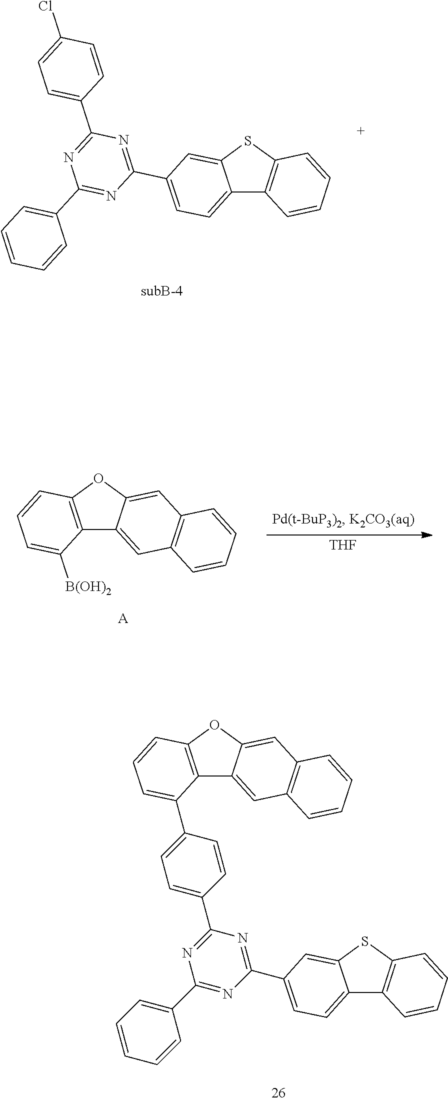

Compound subB-4 (15 g, 33.3 mmol) and Compound A (9.6 g, 36.7 mmol) were added to THF (300 ml) under a nitrogen atmosphere, and the mixture was stirred and refluxed. Then, potassium carbonate (13.8 g, 100 mmol) was dissolved in water (41 ml), added thereto, and the mixture was sufficiently stirred and then bis(tri-tert-butylphosphine)palladium(0) (0.2 g, 0.3 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 26 (15.8 g). (Yield: 75%, MS: [M+H]⁺=632)

### Preparation Example 1-27

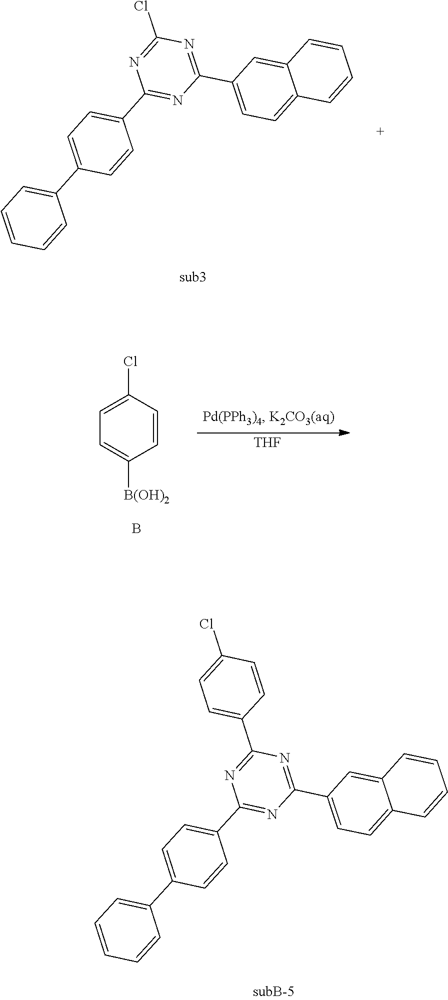

Compound sub3 (15 g, 38.1 mmol) and Compound B (10 g, 38.1 mmol) were added to THF (300 ml) under a nitrogen atmosphere, and the mixture was stirred and refluxed. Then, potassium carbonate (15.8 g, 114.3 mmol) was dissolved in water (47 ml), added thereto, and the mixture was sufficiently stirred and then tetrakis(triphenylphosphine)palladium(0) (0.4 g, 0.4 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subB-5 (14.1 g). (Yield: 79%, MS: [M+H]⁺=470)

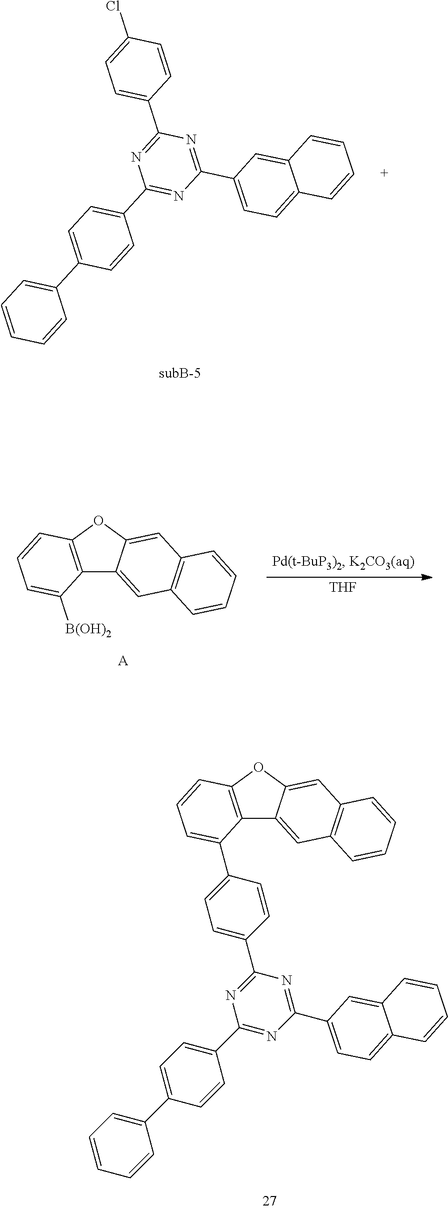

Compound subB-5 (15 g, 31.9 mmol) and Compound A (9.2 g, 35.1 mmol) were added to THF (300 ml) under a nitrogen atmosphere, and the mixture was stirred and refluxed. Then, potassium carbonate (13.2 g, 95.8 mmol) was dissolved in water (40 ml), added thereto, and the mixture was sufficiently stirred and then bis(tri-tert-butylphosphine)palladium(0) (0.2 g, 0.3 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 27 (12.5 g). (Yield: 60%, MS: [M+H]⁺=652)

### Preparation Example 1-28

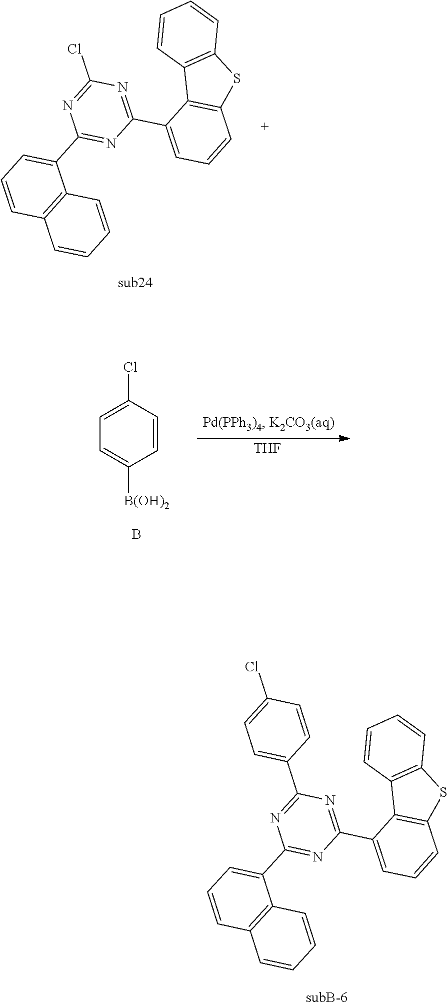

Compound sub24 (15 g, 35.4 mmol) and Compound B (5.5 g, 35.4 mmol) were added to THF (300 ml) under a nitrogen atmosphere, and the mixture was stirred and refluxed. Then, potassium carbonate (14.7 g, 106.2 mmol) was dissolved in water (44 ml), added thereto, and the mixture was sufficiently stirred and then tetrakis(triphenylphosphine)palladium(0) (0.4 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subB-6 (12.5 g). (Yield: 71%, MS: [M+H]⁺=500)

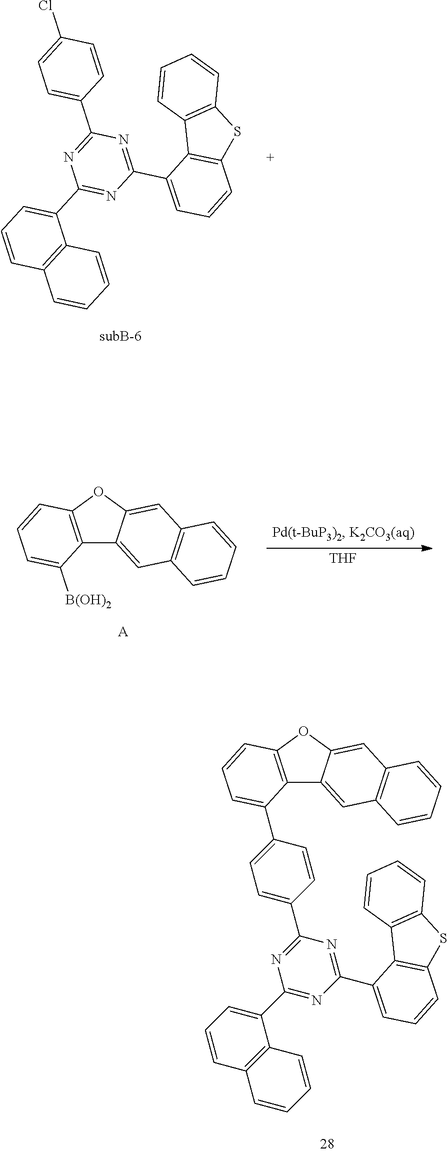

Compound subB-6 (15 g, 30 mmol) and Compound A (8.6 g, 33 mmol) were added to THF (300 ml) under a nitrogen atmosphere, and the mixture was stirred and refluxed. Then, potassium carbonate (12.4 g, 90 mmol) was dissolved in water (37 ml), added thereto, and the mixture was sufficiently stirred and then bis(tri-tert-butylphosphine)palladium(0) (0.2 g, 0.3 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 28 (14.9 g). (Yield: 73%, MS: [M+H]⁺=682)

### Preparation Example 1-29

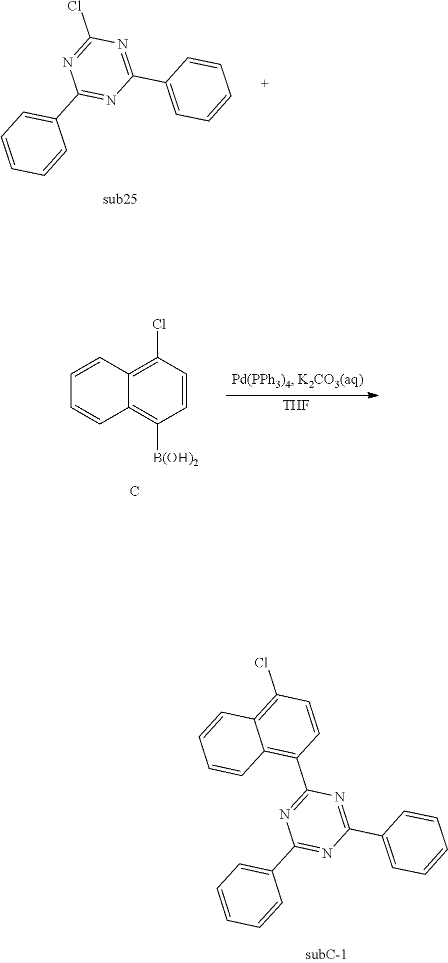

Compound sub25 (15 g, 56 mmol) and Compound C (11.6 g, 56 mmol) were added to THF (300 ml) under a nitrogen atmosphere, and the mixture was stirred and refluxed. Then, potassium carbonate (23.2 g, 168.1 mmol) was dissolved in water (70 ml), added thereto, and the mixture was sufficiently stirred and then tetrakis(triphenylphosphine)palladium(0) (0.6 g, 0.6 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-1 (16.7 g). (Yield: 76%, MS: [M+H]⁺=394)

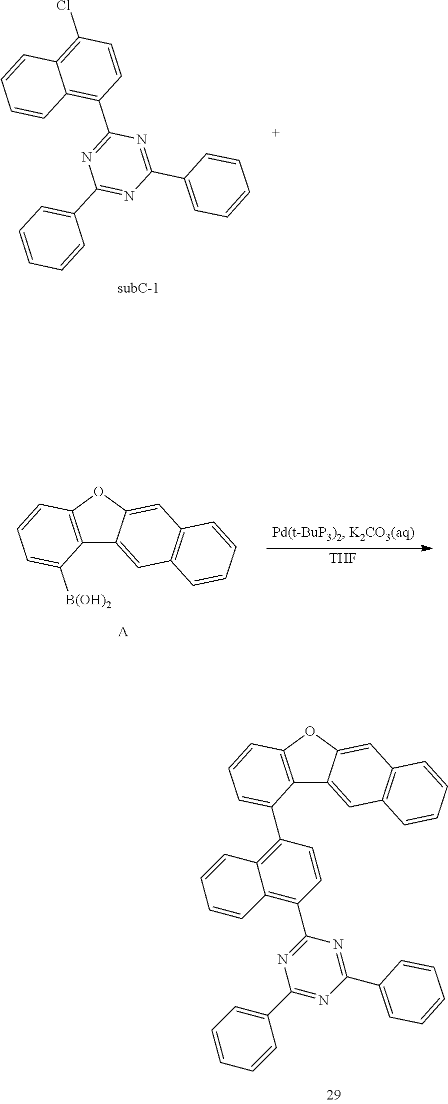

Compound subC-1 (15 g, 38.1 mmol) and Compound A (10 g, 38.1 mmol) were added to THF (300 ml) under a nitrogen atmosphere, and the mixture was stirred and refluxed. Then, potassium carbonate (15.8 g, 114.3 mmol) was dissolved in water (47 ml), added thereto, and the mixture was sufficiently stirred and then bis(tri-tert-butylphosphine)palladium(0) (0.2 g, 0.4 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 29 (16 g). (Yield: 73%, MS: [M+H]⁺=576)

### Preparation Example 1-30

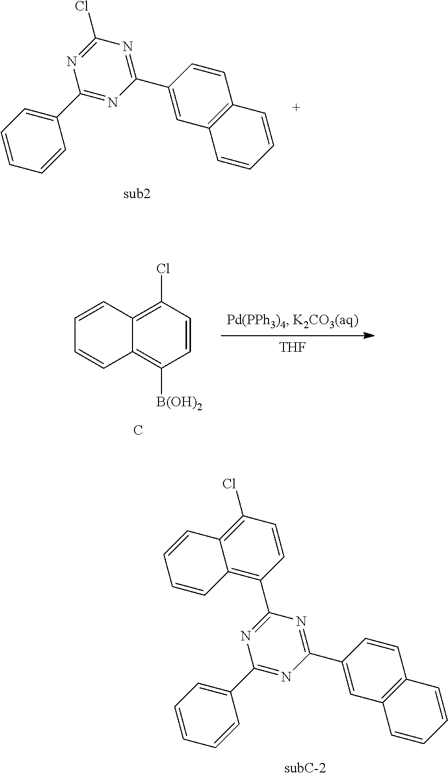

Compound sub2 (15 g, 47.2 mmol) and Compound C (9.7 g, 47.2 mmol) were added to THF (300 ml) under a nitrogen atmosphere, and the mixture was stirred and refluxed. Then, potassium carbonate (19.6 g, 141.6 mmol) was dissolved in water (59 ml), added thereto, and the mixture was sufficiently stirred and then tetrakis(triphenylphosphine)palladium(0) (0.5 g, 0.5 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-2 (14 g). (Yield: 67%, MS: [M+H]⁺=444)

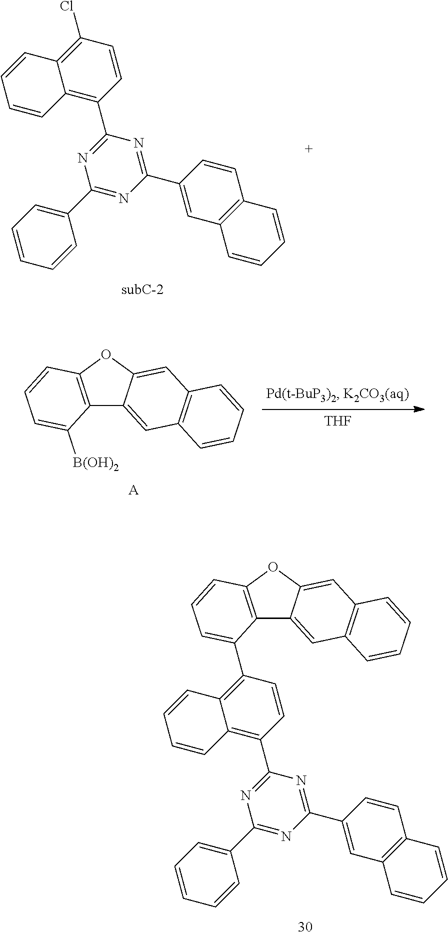

Compound subC-2 (15 g, 33.8 mmol) and Compound A (8.9 g, 33.8 mmol) were added to THF (300 ml) under a nitrogen atmosphere, and the mixture was stirred and refluxed. Then, potassium carbonate (14 g, 101.4 mmol) was dissolved in water (42 ml), added thereto, and the mixture was sufficiently stirred and then bis(tri-tert-butylphosphine)palladium(0) (0.2 g, 0.3 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 30 (13.1 g). (Yield: 62%, MS: [M+H]⁺=626)

### Preparation Example 1-31

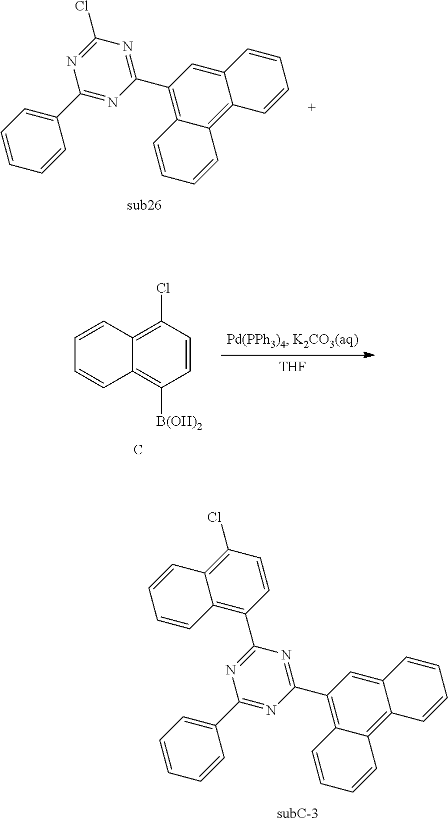

Compound sub26 (15 g, 40.8 mmol) and Compound C (8.4 g, 40.8 mmol) were added to THF (300 ml) under a nitrogen atmosphere, and the mixture was stirred and refluxed. Then, potassium carbonate (16.9 g, 122.3 mmol) was dissolved in water (51 ml), added thereto, and the mixture was sufficiently stirred and then tetrakis(triphenylphosphine)palladium(0) (0.5 g, 0.4 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-3 (13.5 g). (Yield: 67%, MS: [M+H]⁺=494)

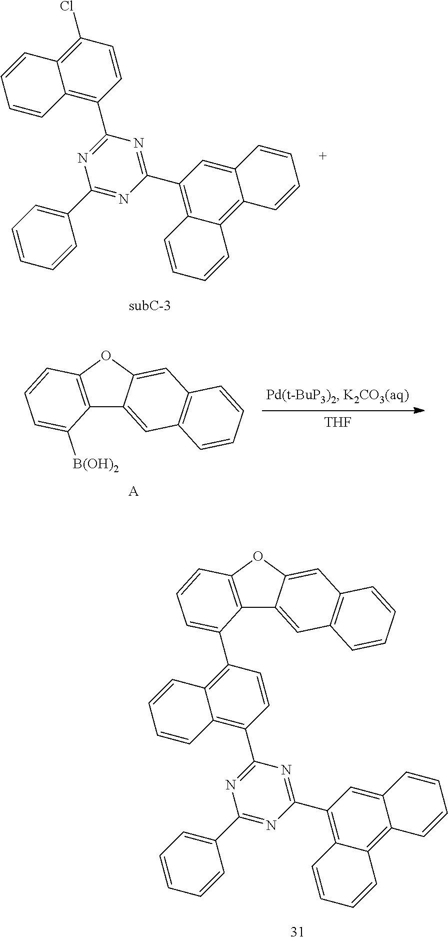

Compound subC-3 (15 g, 30.4 mmol) and Compound A (8 g, 30.4 mmol) were added to THF (300 ml) under a nitrogen atmosphere, and the mixture was stirred and refluxed. Then, potassium carbonate (12.6 g, 91.1 mmol) was dissolved in water (38 ml), added thereto, and the mixture was sufficiently stirred and then bis(tri-tert-butylphosphine)palladium(0) (0.2 g, 0.3 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 31 (15.6 g). (Yield: 76%, MS: [M+H]⁺=676)

### Preparation Example 1-32

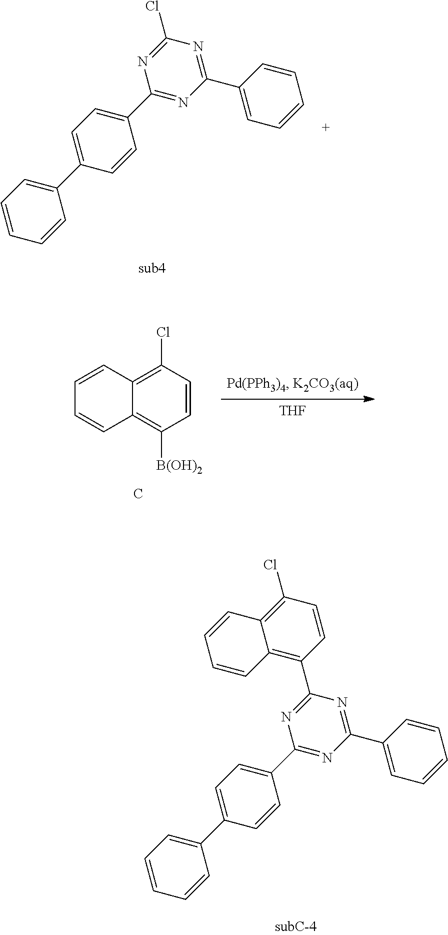

Compound sub4 (15 g, 43.6 mmol) and Compound C (9 g, 43.6 mmol) were added to THF (300 ml) under a nitrogen atmosphere, and the mixture was stirred and refluxed. Then, potassium carbonate (18.1 g, 130.9 mmol) was dissolved in water (54 ml), added thereto, and the mixture was sufficiently stirred and then tetrakis(triphenylphosphine)palladium(0) (0.5 g, 0.4 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-4 (16.4 g). (Yield: 80%, MS: [M+H]⁺=470)

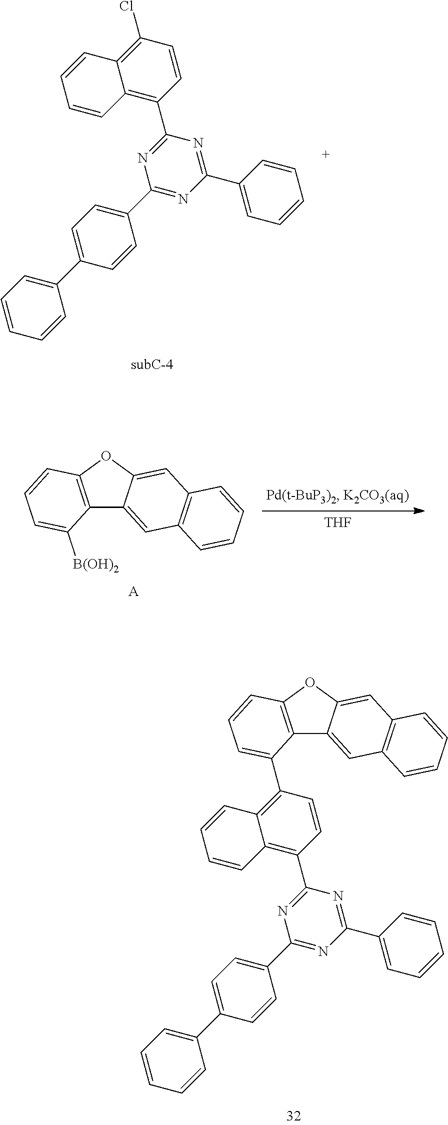

Compound subC-4 (15 g, 31.9 mmol) and Compound A (8.4 g, 31.9 mmol) were added to THF (300 ml) under a nitrogen atmosphere, and the mixture was stirred and refluxed. Then, potassium carbonate (13.2 g, 95.8 mmol) was dissolved in water (40 ml), added thereto, and the mixture was sufficiently stirred and then bis(tri-tert-butylphosphine)palladium(0) (0.2 g, 0.3 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 32 (13.5 g). (Yield: 65%, MS: [M+H]⁺=652)

### Preparation Example 1-33

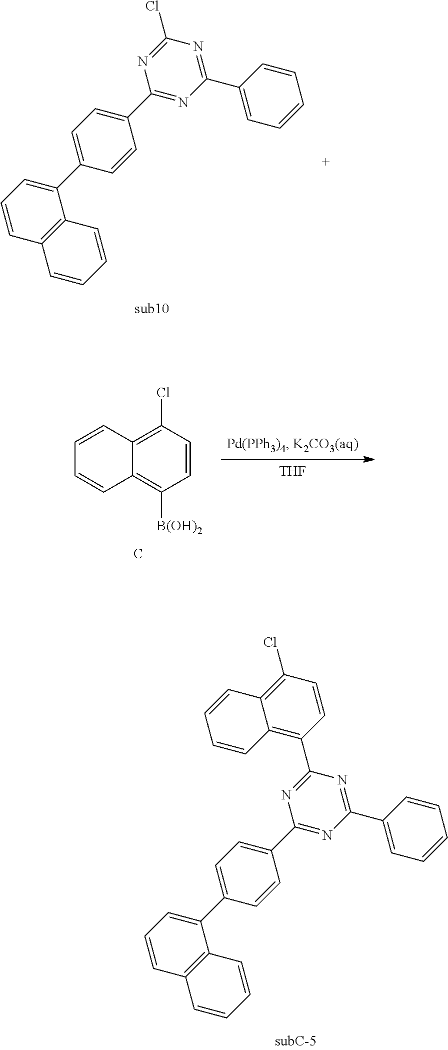

Compound sub10 (15 g, 38.1 mmol) and Compound C (7.9 g, 38.1 mmol) were added to THF (300 ml) under a nitrogen atmosphere, and the mixture was stirred and refluxed. Then, potassium carbonate (15.8 g, 114.3 mmol) was dissolved in water (47 ml), added thereto, and the mixture was sufficiently stirred and then tetrakis(triphenylphosphine)palladium(0) (0.4 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-5 (14.2 g). (Yield: 72%, MS: [M+H]⁺=520)

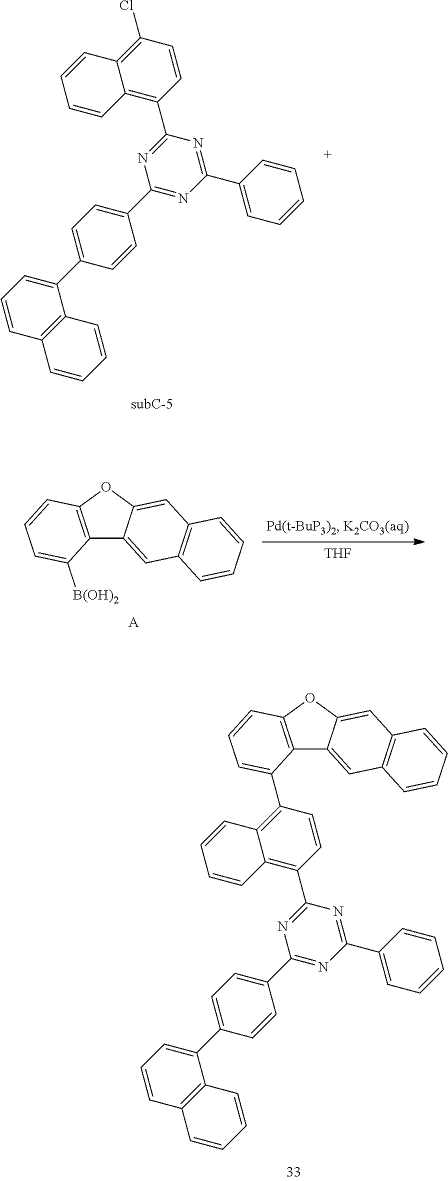

Compound subC-5 (15 g, 28.8 mmol) and Compound A (7.6 g, 28.8 mmol) were added to THF (300 ml) under a nitrogen atmosphere, and the mixture was stirred and refluxed. Then, potassium carbonate (12 g, 86.5 mmol) was dissolved in water (36 ml), added thereto, and the mixture was sufficiently stirred and then bis(tri-tert-butylphosphine)palladium(0) (0.1 g, 0.3 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 33 (12.1 g). (Yield: 60%, MS: [M+H]⁺=702)

### Preparation Example 1-34

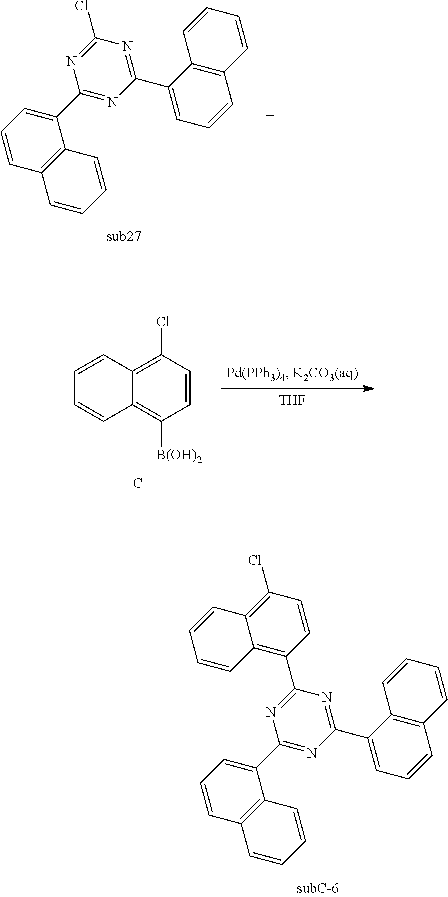

Compound sub27 (15 g, 40.8 mmol) and Compound C (8.4 g, 40.8 mmol) were added to THF (300 ml) under a nitrogen atmosphere, and the mixture was stirred and refluxed. Then, potassium carbonate (16.9 g, 122.3 mmol) was dissolved in water (51 ml), added thereto, and the mixture was sufficiently stirred and then tetrakis(triphenylphosphine)palladium (0) (0.5 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-6 (15.7 g). (Yield: 78%, MS: [M+H]⁺=494)

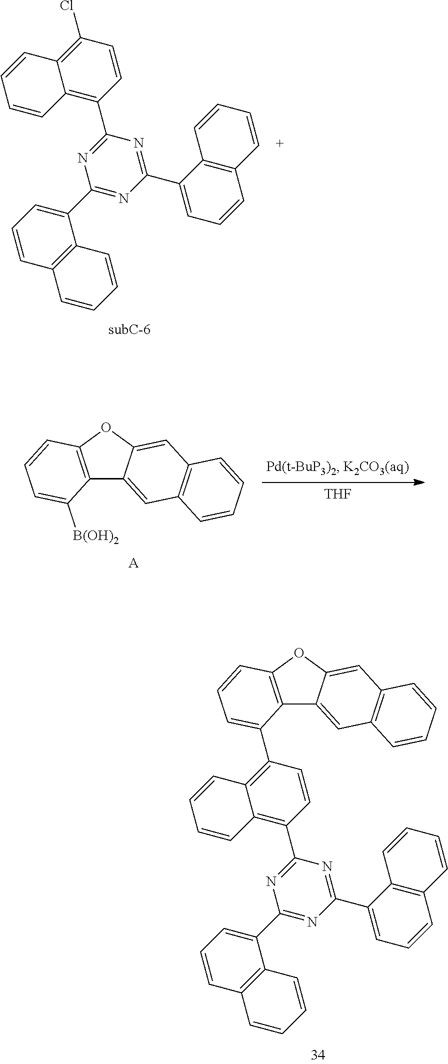

Compound subC-6 (15 g, 30.4 mmol) and Compound A (8 g, 30.4 mmol) were added to THF (300 ml) under a nitrogen atmosphere, and the mixture was stirred and refluxed. Then, potassium carbonate (12.6 g, 91.1 mmol) was dissolved in water (38 ml), added thereto, and the mixture was sufficiently stirred and then bis(tri-tert-butylphosphine)palladium(0) (0.2 g, 0.3 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 34 (15.2 g). (Yield: 74%, MS: [M+H]⁺=676)

### Preparation Example 1-35

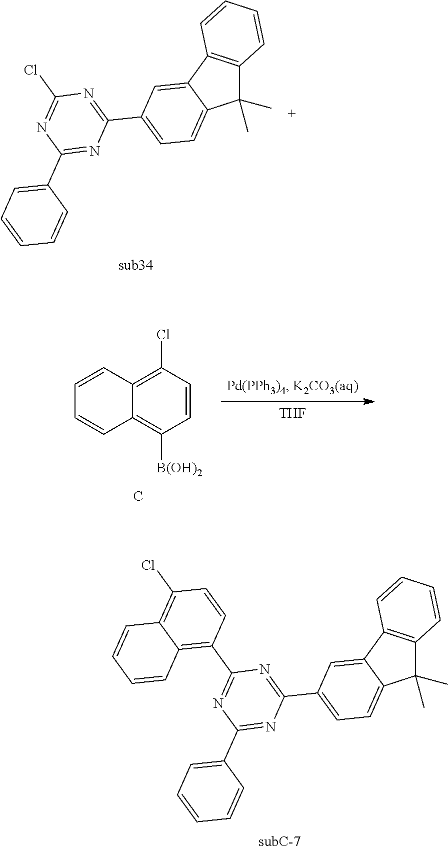

Compound sub34 (15 g, 39.1 mmol) and Compound C (8.1 g, 39.1 mmol) were added to THF (300 ml) under a nitrogen atmosphere, and the mixture was stirred and refluxed. Then, potassium carbonate (16.2 g, 117.2 mmol) was dissolved in water (49 ml), added thereto, and the mixture was sufficiently stirred and then tetrakis(triphenylphosphine)palladium (0) (0.5 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-7 (15.9 g). (Yield: 80%, MS: [M+H]⁺=510)

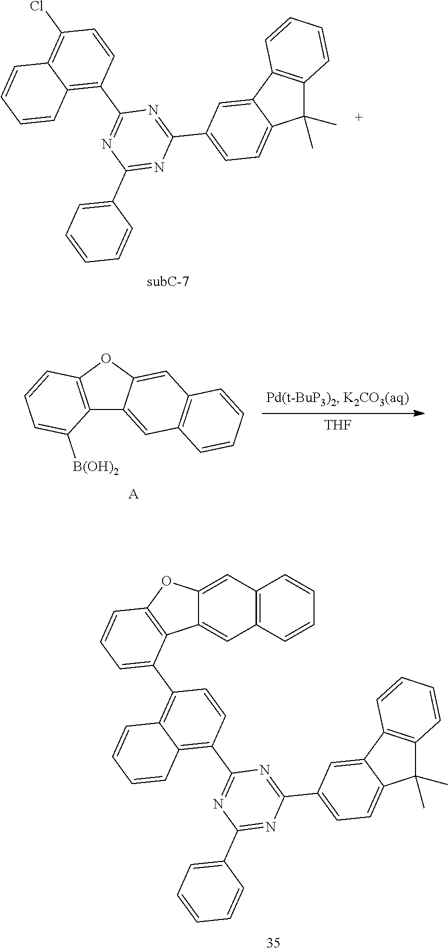

Compound subC-7 (15 g, 29.4 mmol) and Compound A (7.7 g, 29.4 mmol) were added to THF (300 ml) under a nitrogen atmosphere, and the mixture was stirred and refluxed. Then, potassium carbonate (12.2 g, 88.2 mmol) was dissolved in water (37 ml), added thereto, and the mixture was sufficiently stirred and then bis(tri-tert-butylphosphine)palladium(0) (0.2 g, 0.3 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 35 (14.2 g). (Yield: 70%, MS: [M+H]⁺=692)

### Preparation Example 1-38

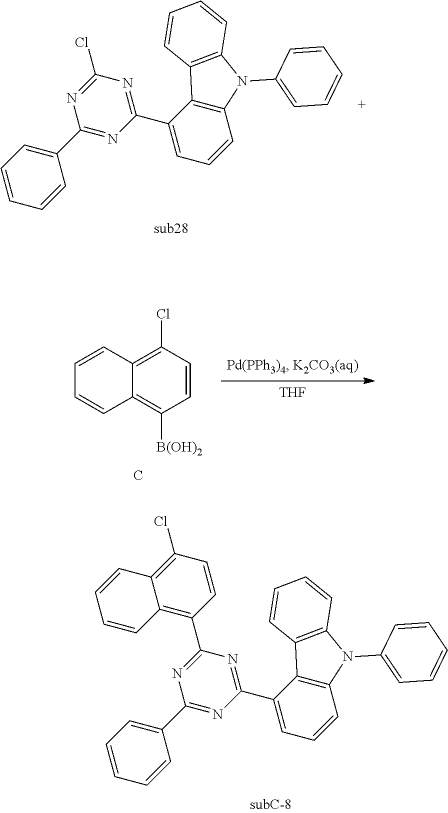

Compound sub28 (15 g, 34.6 mmol) and Compound C (7.2 g, 34.6 mmol) were added to THF (300 ml) under a nitrogen atmosphere, and the mixture was stirred and refluxed. Then, potassium carbonate (14.4 g, 103.9 mmol) was dissolved in water (43 ml), added thereto, and the mixture was sufficiently stirred and then tetrakis(triphenylphosphine)palladium (0) (0.4 g, 0.3 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-8 (13.3 g). (Yield: 69%, MS: [M+H]⁺=559)

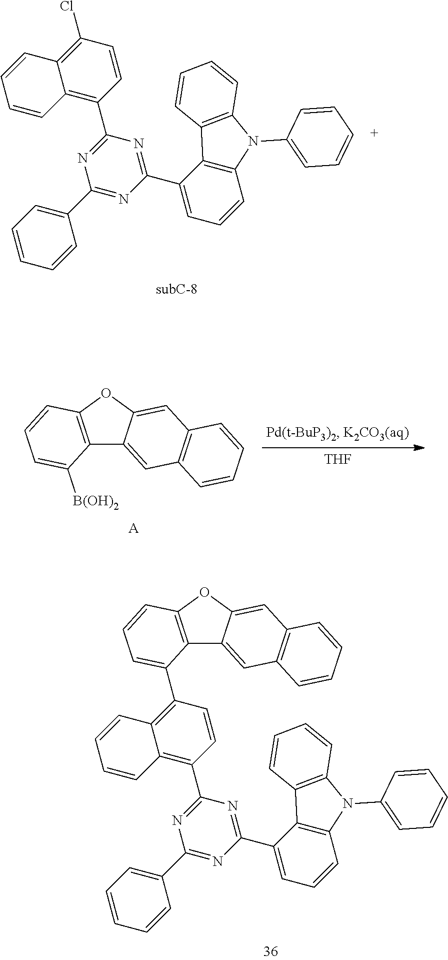

Compound subC-8 (15 g, 26.8 mmol) and Compound A (7 g, 26.8 mmol) were added to THF (300 ml) under a nitrogen atmosphere, and the mixture was stirred and refluxed. Then, potassium carbonate (11.1 g, 80.5 mmol) was dissolved in water (33 ml), added thereto, and the mixture was sufficiently stirred and then bis(tri-tert-butylphosphine)palladium(0) (0.1 g, 0.3 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 36 (15.5 g). (Yield: 78%, MS: [M+H]⁺=741)

### Preparation Example 1-37

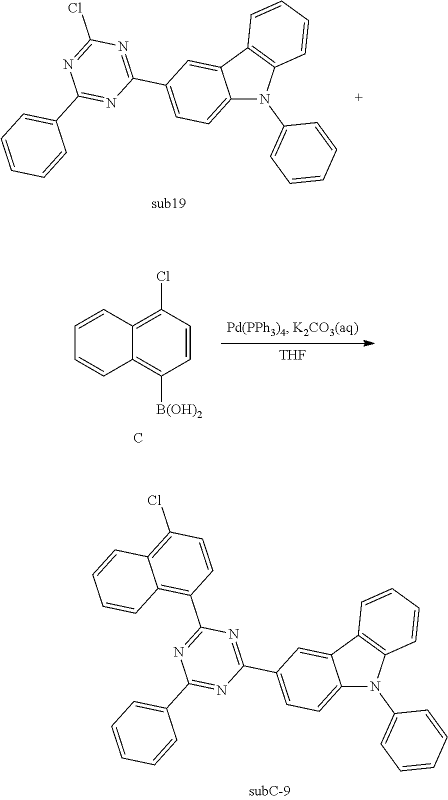

Compound sub19 (15 g, 34.6 mmol) and Compound C (7.2 g, 34.6 mmol) were added to THF (300 ml) under a nitrogen atmosphere, and the mixture was stirred and refluxed. Then, potassium carbonate (14.4 g, 103.9 mmol) was dissolved in water (43 ml), added thereto, and the mixture was sufficiently stirred and then tetrakis(triphenylphosphine)palladium(0) (0.4 g, 0.3 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-9 (13.9 g). (Yield: 72%, MS: [M+H]⁺=559)

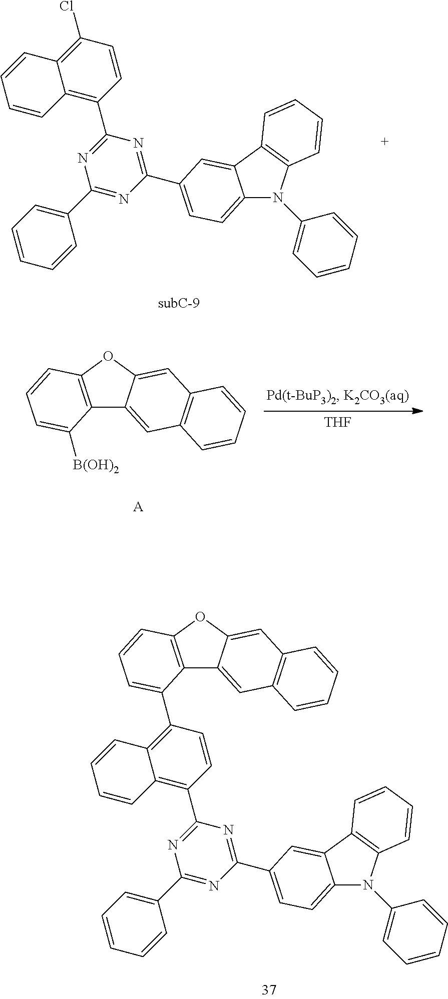

Compound subC-9 (15 g, 26.8 mmol) and Compound A (7 g, 26.8 mmol) were added to THF (300 ml) under a nitrogen atmosphere, and the mixture was stirred and refluxed. Then, potassium carbonate (11.1 g, 80.5 mmol) was dissolved in water (33 ml), added thereto, and the mixture was sufficiently stirred and then bis(tri-tert-butylphosphine)palladium(0) (0.1 g, 0.3 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 37 (14.5 g). (Yield: 73%, MS: [M+H]⁺=741)

### Preparation Example 1-38

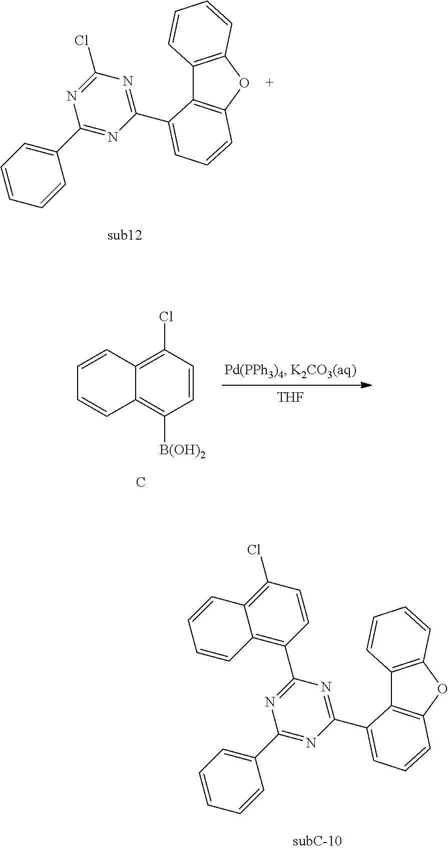

Compound sub12 (15 g, 41.9 mmol) and Compound C (8.7 g, 41.9 mmol) were added to THF (300 ml) under a nitrogen atmosphere, and the mixture was stirred and refluxed. Then, potassium carbonate (17.4 g, 125.8 mmol) was dissolved in water (52 ml), added thereto, and the mixture was sufficiently stirred and then tetrakis(triphenylphosphine)palladium(0) (0.5 g, 0.4 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-10 (14.2 g). (Yield: 70%, MS: [M+H]⁺=484)

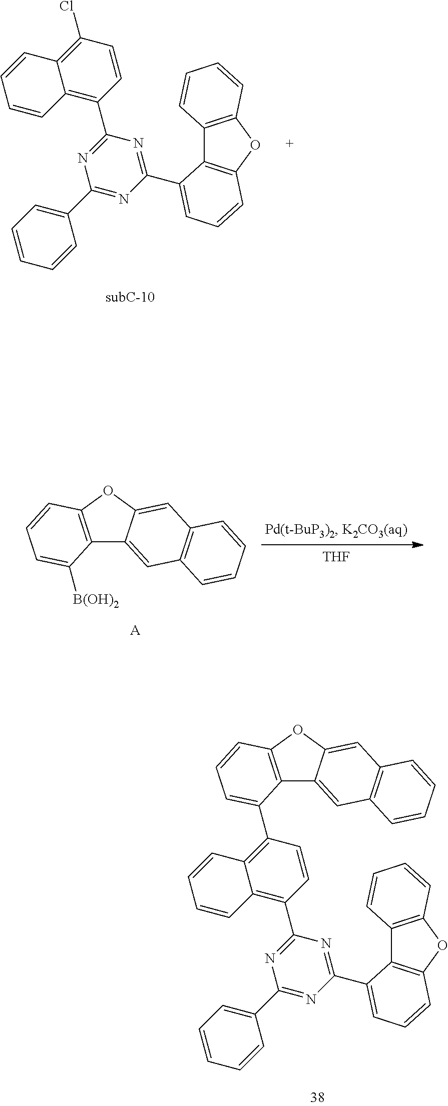

Compound subC-10 (15 g, 31 mmol) and Compound A (8.1 g, 31 mmol) were added to THF (300 ml) under a nitrogen atmosphere, and the mixture was stirred and refluxed. Then, potassium carbonate (12.9 g, 93 mmol) was dissolved in water (39 ml), added thereto, and the mixture was sufficiently stirred and then bis(tri-tert-butylphosphine)palladium (0) (0.2 g, 0.3 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 38 (13.4 g). (Yield: 65%, MS: [M+H]⁺=666)

### Preparation Example 1-39

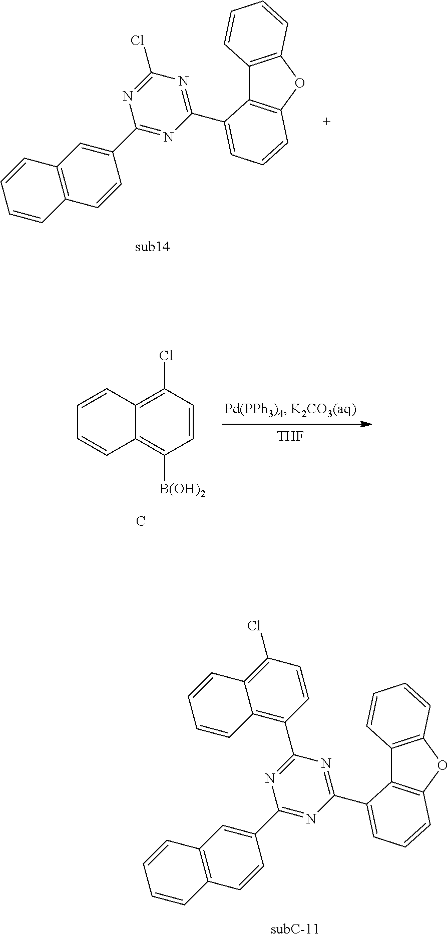

Compound sub14 (15 g, 36.8 mmol) and Compound C (7.6 g, 36.8 mmol) were added to THF (300 ml) under a nitrogen atmosphere, and the mixture was stirred and refluxed. Then, potassium carbonate (15.2 g, 110.3 mmol) was dissolved in water (46 ml), added thereto, and the mixture was sufficiently stirred and then tetrakis(triphenylphosphine)palladium(0) (0.4 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-11 (12.9 g). (Yield: 66%, MS: [M+H]⁺=534)

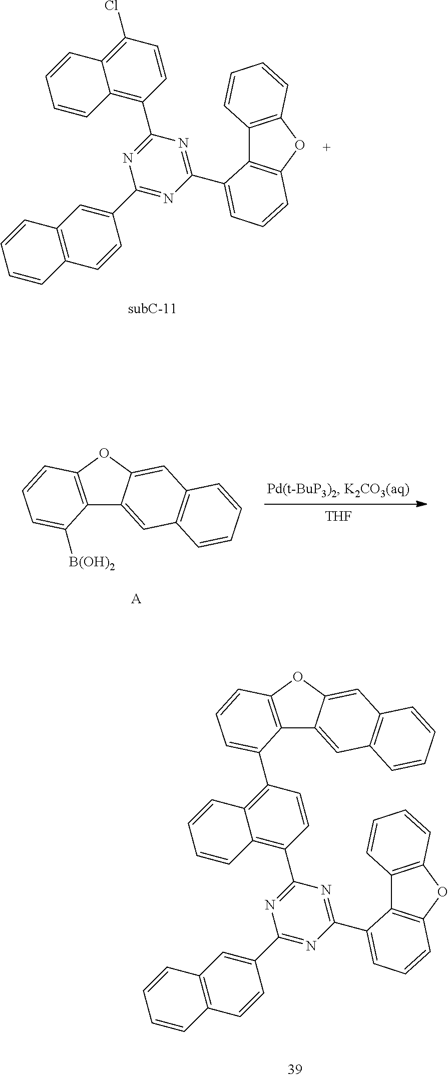

Compound subC-11 (15 g, 28.1 mmol) and Compound A (7.4 g, 28.1 mmol) were added to THF (300 ml) under a nitrogen atmosphere, and the mixture was stirred and refluxed. Then, potassium carbonate (11.6 g, 84.3 mmol) was dissolved in water (35 ml), added thereto, and the mixture was sufficiently stirred and then bis(tri-tert-butylphosphine)palladium(0) (0.1 g, 0.3 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 39 (14.5 g). (Yield: 72%, MS: [M+H]⁺=716)

### Preparation Example 1-40

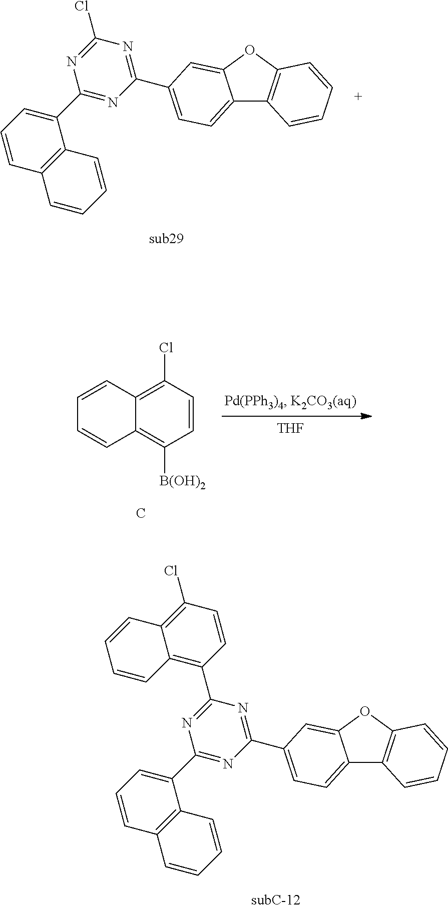

Compound sub29 (15 g, 36.8 mmol) and Compound C (7.6 g, 36.8 mmol) were added to THF (300 ml) under a nitrogen atmosphere, and the mixture was stirred and refluxed. Then, potassium carbonate (15.2 g, 110.3 mmol) was dissolved in water (46 ml), added thereto, and the mixture was sufficiently stirred and then tetrakis(triphenylphosphine)palladium(0) (0.4 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-12 (12.9 g). (Yield: 66%, MS: [M+H]⁺=534)

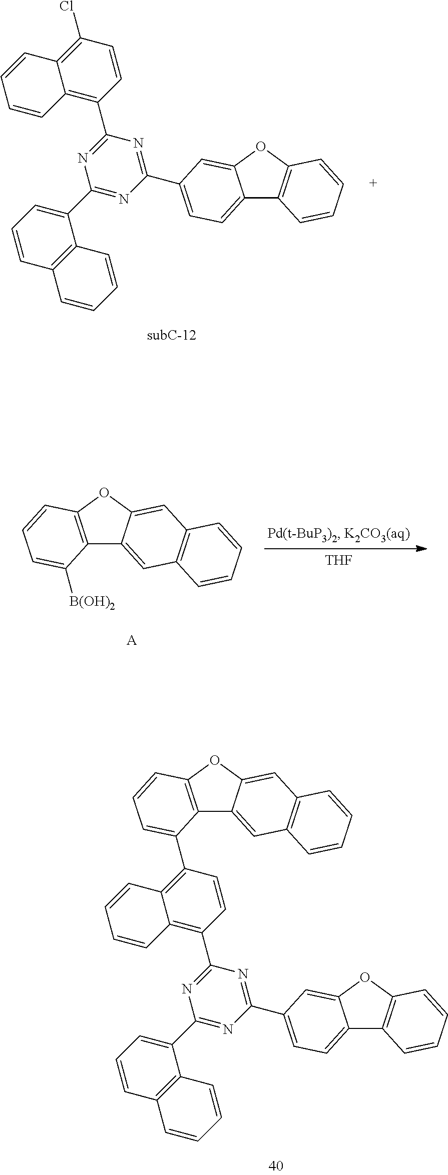

Compound subC-12 (15 g, 28.1 mmol) and Compound A (7.4 g, 28.1 mmol) were added to THF (300 ml) under a nitrogen atmosphere, and the mixture was stirred and refluxed. Then, potassium carbonate (11.6 g, 84.3 mmol) was dissolved in water (35 ml), added thereto, and the mixture was sufficiently stirred and then bis(tri-tert-butylphosphine)palladium(0) (0.1 g, 0.3 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 40 (12.7 g). (Yield: 63%, MS: [M+H]⁺=716)

### Preparation Example 1-41

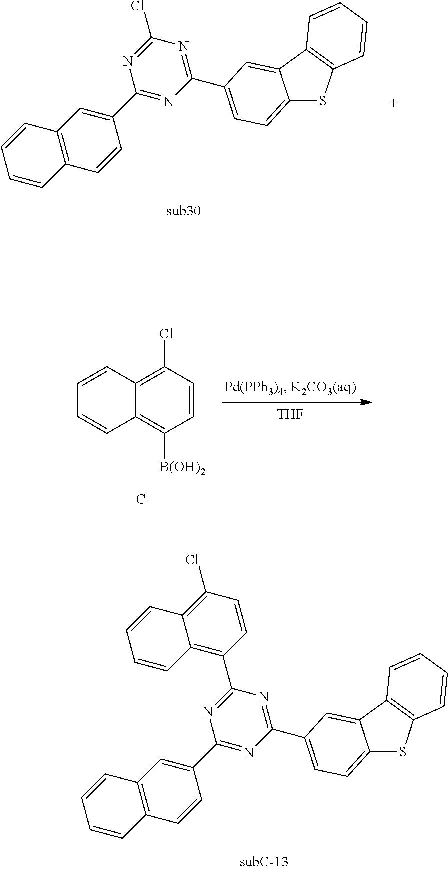

Compound sub30 (15 g, 35.5 mmol) and Compound C (7.3 g, 35.5 mmol) were added to THF (300 ml) under a nitrogen atmosphere, and the mixture was stirred and refluxed. Then, potassium carbonate (14.7 g, 106.4 mmol) was dissolved in water (44 ml), added thereto, and the mixture was sufficiently stirred and then tetrakis(triphenylphosphine)palladium(0) (0.4 g, 0.4 mmol) was added. After the reaction for 12 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-13 (14.6 g). (Yield: 75%, MS: [M+H]⁺=550)

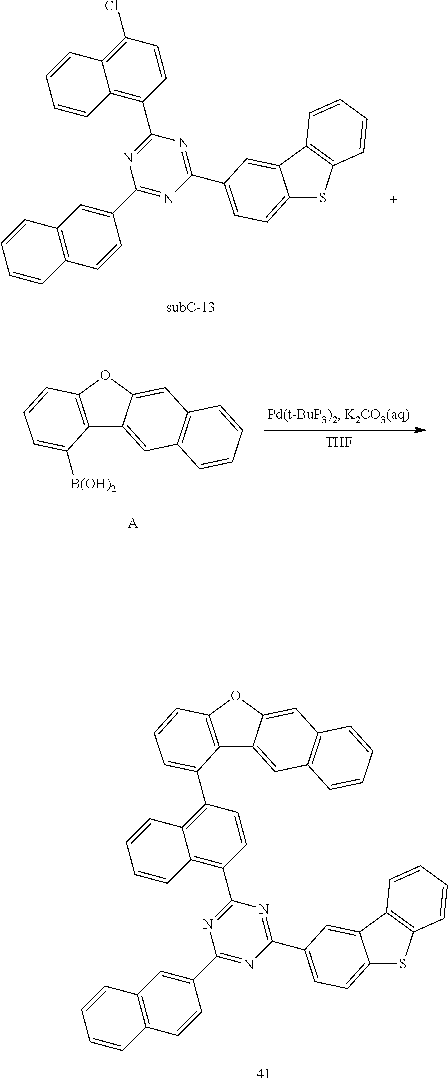

Compound subC-13 (15 g, 27.3 mmol) and Compound A (7.1 g, 27.3 mmol) were added to THF (300 ml) under a nitrogen atmosphere, and the mixture was stirred and refluxed. Then, potassium carbonate (11.3 g, 81.8 mmol) was dissolved in water (34 ml), added thereto, and the mixture was sufficiently stirred and then bis(tri-tert-butylphosphine)palladium(0) (0.1 g, 0.3 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 41 (13.6 g). (Yield: 68%, MS: [M+H]⁺=732)

### Preparation Example 1-42

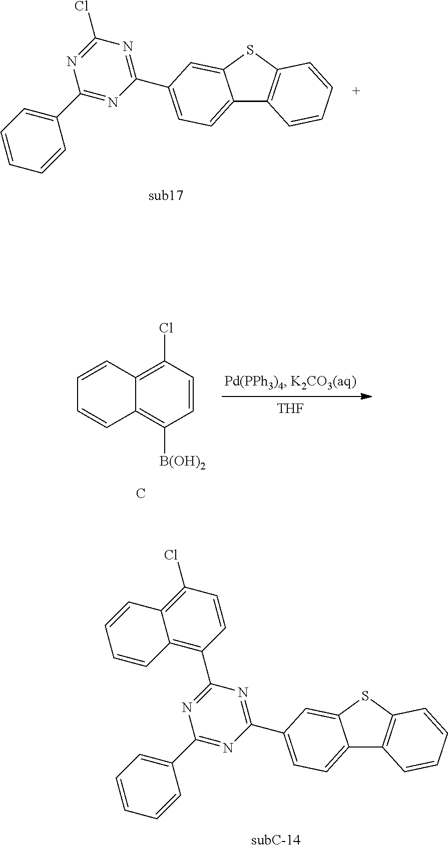

Compound sub17 (15 g, 40.1 mmol) and Compound C (8.3 g, 40.1 mmol) were added to THF (300 ml) under a nitrogen atmosphere, and the mixture was stirred and refluxed. Then, potassium carbonate (16.6 g, 120.4 mmol) was dissolved in water (50 ml), added thereto, and the mixture was sufficiently stirred and then tetrakis(triphenylphosphine)palladium(0) (0.5 g, 0.4 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subC-14 (13 g). (Yield: 65%, MS: [M+H]⁺=500)

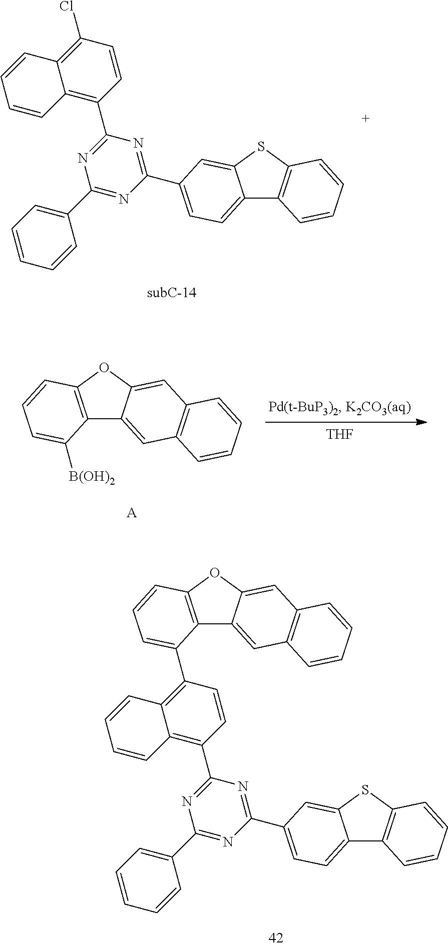

Compound subC-14 (15 g, 30 mmol) and Compound A (7.9 g, 30 mmol) were added to THF (300 ml) under a nitrogen atmosphere, and the mixture was stirred and refluxed. Then, potassium carbonate (12.4 g, 90 mmol) was dissolved in water (37 ml), added thereto, and the mixture was sufficiently stirred and then bis(tri-tert-butylphosphine)palladium(0) (0.2 g, 0.3 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound 42 (14.7 g). (Yield: 72%, MS: [M+H]⁺=682)

### Preparation Example 2-1

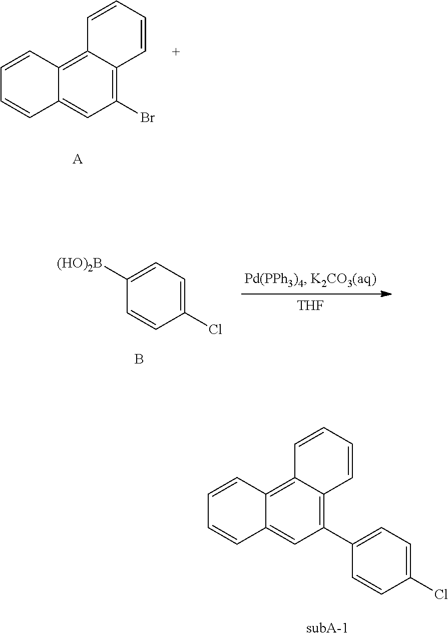

Compound A (15 g, 58.3 mmol) and Compound B (10 g, 64.2 mmol) were added to THF (300 ml) under a nitrogen atmosphere, and the mixture was stirred and refluxed. Then, potassium carbonate (24.2 g, 175 mmol) was dissolved in water (73 ml), added thereto, and the mixture was sufficiently stirred and then tetrakis(triphenylphosphine)palladium(0) (0.7 g, 0.6 mmol) was added. After the reaction for 11 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subA-1 (10.4 g). (Yield: 62%, MS: [M+H]⁺=289)

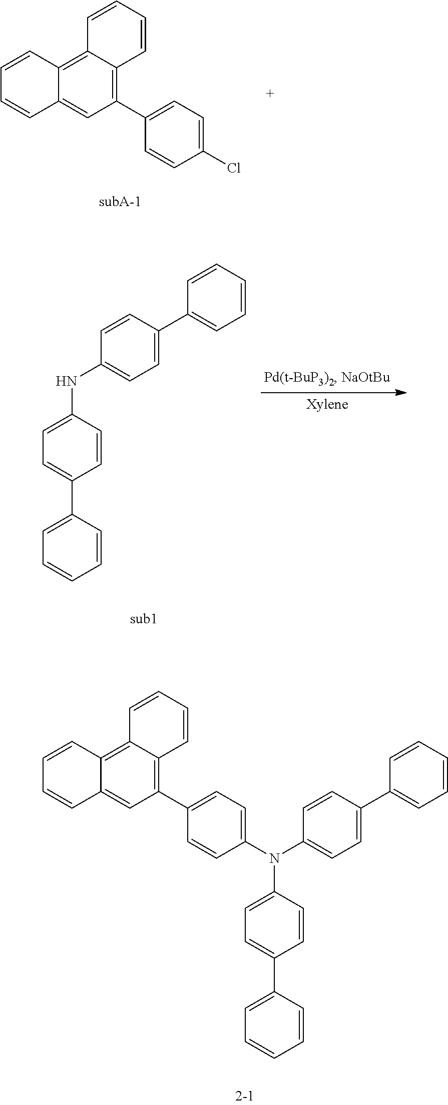 [SMILES: Clc1ccc(-c2cc3ccccc3c3ccccc23)cc1 ; "subA-1" ; c1ccc(-c2ccc(Nc3ccc(-c4ccccc4)cc3)cc2)cc1 ; "Xylene" ; c1ccc(-c2ccc(N(c3ccc(-c4ccccc4)cc3)c3ccc(-c4cc5ccccc5c5ccccc45)cc3)cc2)cc1]

Compound subA-1 (10 g, 34.6 mmol), Compound sub1 (11.1 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. After the reaction for 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-1 (13.3 g). (Yield: 67%, MS: [M+H]⁺=574)

### Preparation Example 2-2

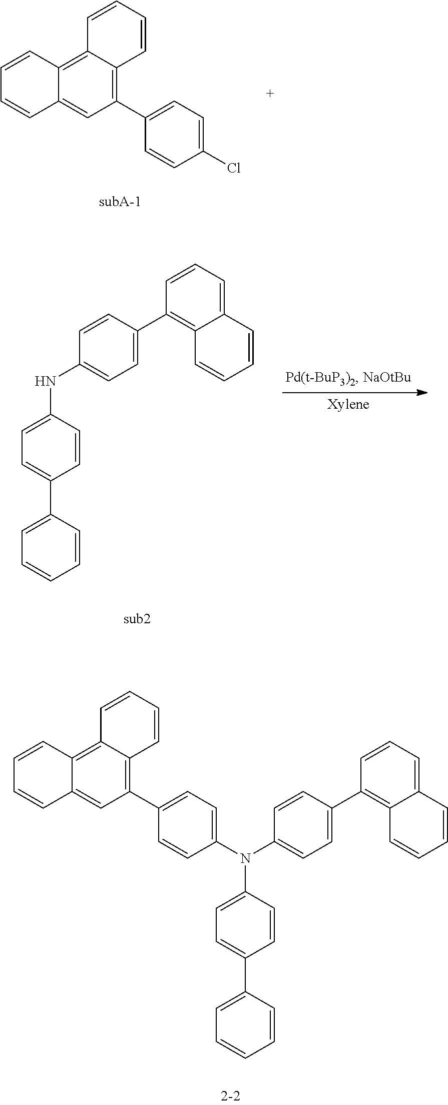 [SMILES: Clc1ccc(-c2cc3ccccc3c3ccccc23)cc1 ; "subA-1" ; c1ccc(-c2ccc(Nc3ccc(-c4cccc5ccccc45)cc3)cc2)cc1 ; "Xylene" ; c1ccc(-c2ccc(N(c3ccc(-c4cccc5ccccc45)cc3)c3ccc(-c4cc5ccccc5c5ccccc45)cc3)cc2)cc1]

Compound subA-1 (10 g, 34.6 mmol), Compound sub2 (12.9 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium (0) (0.2 g, 0.3 mmol) was added thereto, When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-2 (11 g). (Yield: 51%, MS: [M+H]⁺=624)

### Preparation Example 2-3

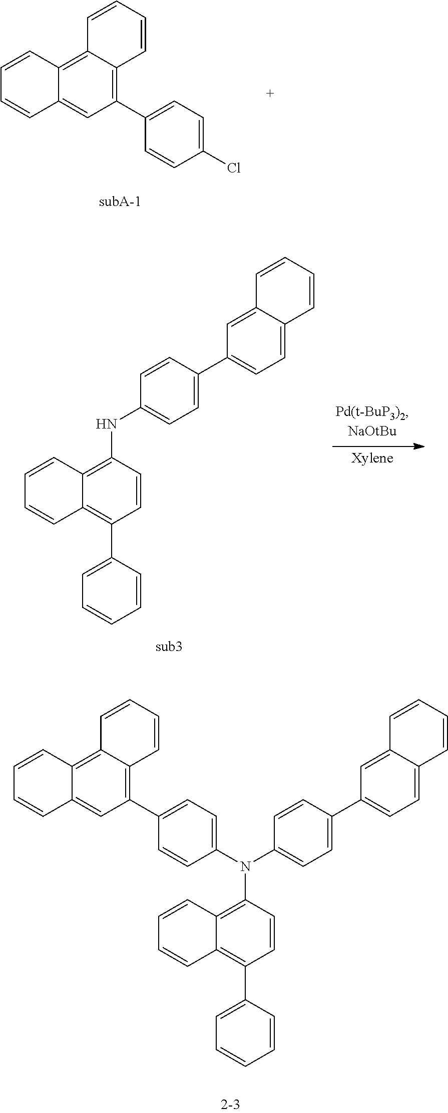 [SMILES: Clc1ccc(-c2cc3ccccc3c3ccccc23)cc1 ; "subA-1" ; c1ccc(-c2ccc(Nc3ccc(-c4ccc5ccccc5c4)cc3)c3ccccc23)cc1 ; "Xylene" ; c1ccc(-c2ccc(N(c3ccc(-c4ccc5ccccc5c4)cc3)c3ccc(-c4cc5ccccc5c5ccccc45)cc3)c3ccccc23)cc1]

Compound subA-1 (10 g, 34.6 mmol), Compound sub3 (14.6 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-3 (14 g). (Yield: 60%, MS: [M+H]⁺=674)

### Preparation Example 2-4

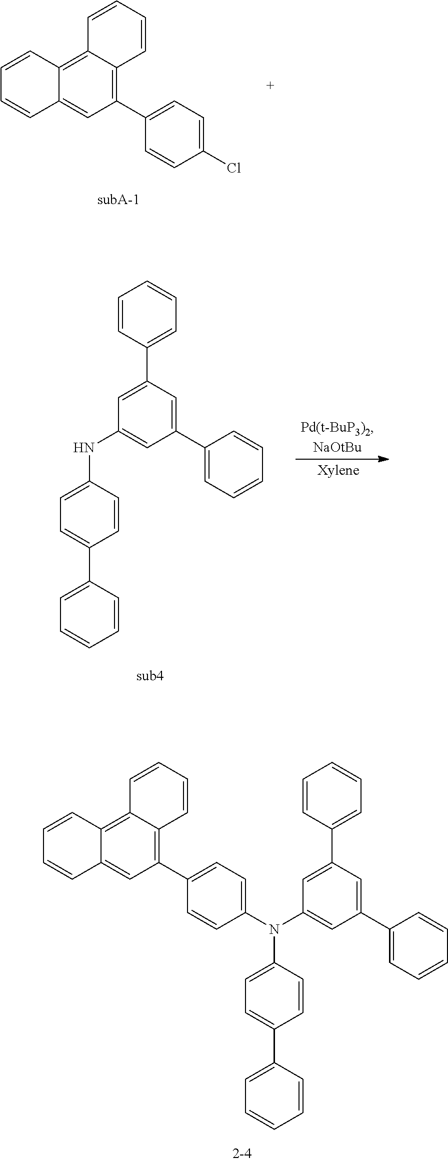 [SMILES: Clc1ccc(-c2cc3ccccc3c3ccccc23)cc1 ; c1ccc(-c2ccc(Nc3cc(-c4ccccc4)cc(-c4ccccc4)c3)cc2)cc1 ; c1ccc(-c2ccc(N(c3ccc(-c4cc5ccccc5c5ccccc45)cc3)c3cc(-c4ccccc4)cc(-c4ccccc4)c3)cc2)cc1]

Compound subA-1 (10 g, 34.6 mmol), Compound sub4 (13.8 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-4 (12.4 g). (Yield: 55%, MS: [M+H]⁺=650)

### Preparation Example 2-5

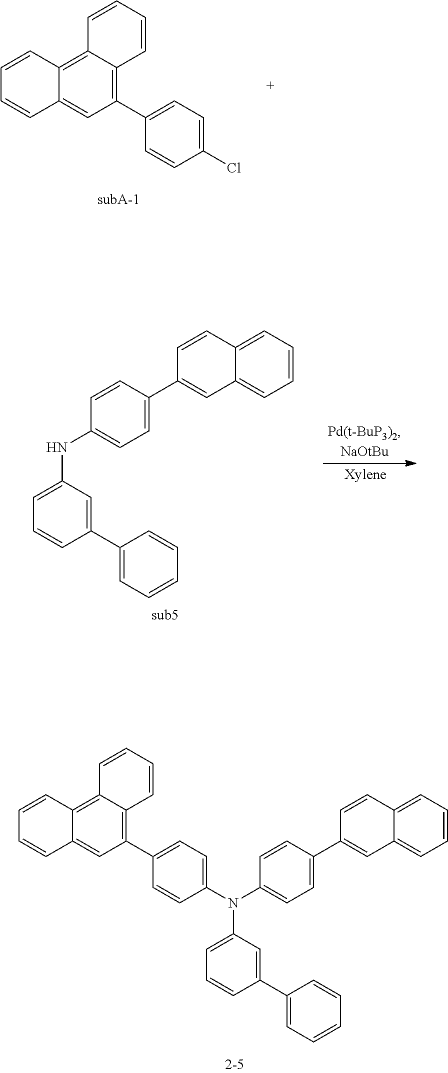 [SMILES: Clc1ccc(-c2cc3ccccc3c3ccccc23)cc1 ; c1ccc(-c2cccc(Nc3ccc(-c4ccc5ccccc5c4)cc3)c2)cc1 ; c1ccc(-c2cccc(N(c3ccc(-c4ccc5ccccc5c4)cc3)c3ccc(-c4cc5ccccc5c5ccccc45)cc3)c2)cc1]

Compound subA-1 (10 g, 34.6 mmol), Compound sub5 (12.9 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-5 (12.7 g). (Yield: 59%, MS: [M+H]⁺=624)

### Preparation Example 2-6

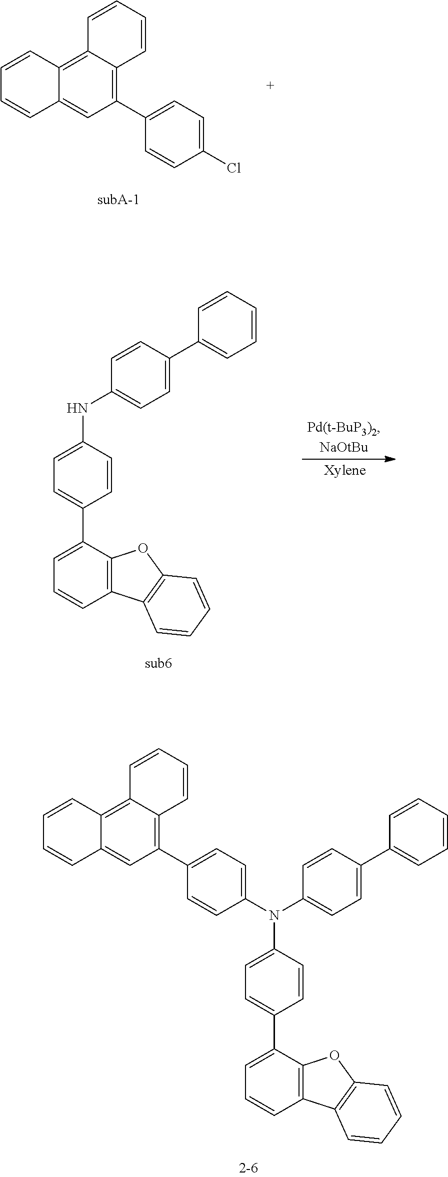 [SMILES: Clc1ccc(-c2cc3ccccc3c3ccccc23)cc1 ; c1ccc(-c2ccc(Nc3ccc(-c4cccc5c4oc4ccccc45)cc3)cc2)cc1 ; c1ccc(-c2ccc(N(c3ccc(-c4cc5ccccc5c5ccccc45)cc3)c3ccc(-c4cccc5c4oc4ccccc45)cc3)cc2)cc1]

Compound subA-1 (10 g, 34.6 mmol), Compound sub6 (14.3 g, 34.6 mmol) and sodium Cert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-6 (15.4 g). (Yield: 67%, MS: [M+H]⁺=664)

### Preparation Example 2-7

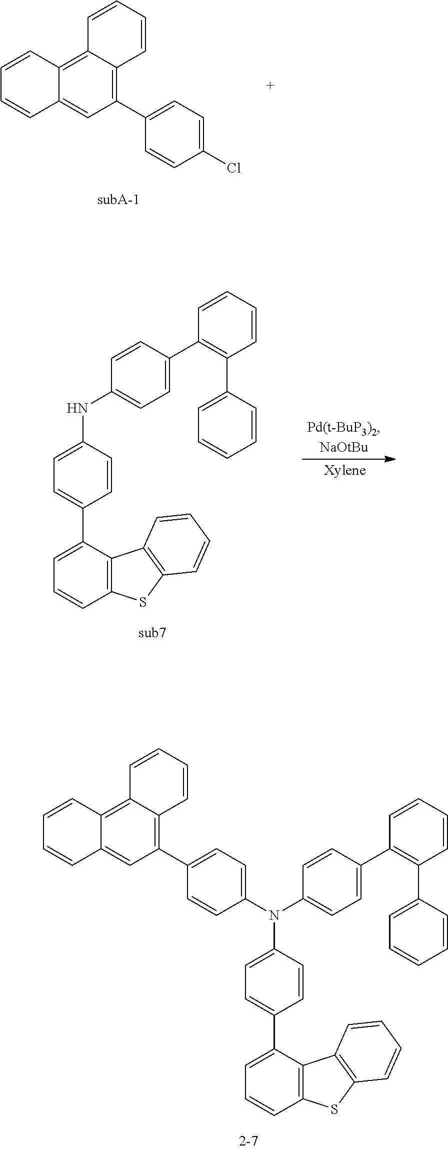 [SMILES: Clc1ccc(-c2cc3ccccc3c3ccccc23)cc1 ; c1ccc(-c2ccccc2-c2ccc(Nc3ccc(-c4cccc5sc6ccccc6c45)cc3)cc2)cc1 ; c1ccc(-c2ccccc2-c2ccc(N(c3ccc(-c4cc5ccccc5c5ccccc45)cc3)c3ccc(-c4cccc5sc6ccccc6c45)cc3)cc2)cc1]

Compound subA-1 (10 g, 34.6 mmol), Compound sub7 (17.4 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium (0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-7 (17.3 g). (Yield: 66%, MS: [M+H]⁺=756)

### Preparation Example 2-8

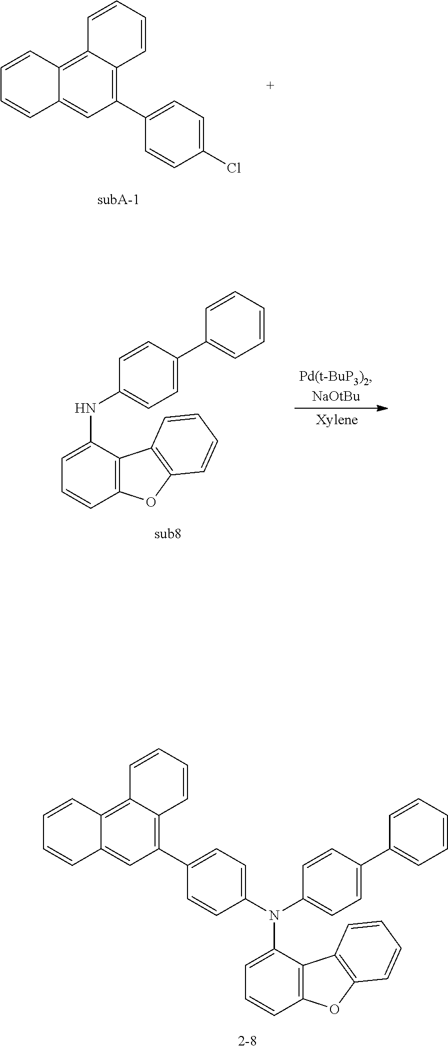 [SMILES: Clc1ccc(-c2cc3ccccc3c3ccccc23)cc1 ; c1ccc(-c2ccc(Nc3cccc4oc5ccccc5c34)cc2)cc1 ; c1ccc(-c2ccc(N(c3ccc(-c4cc5ccccc5c5ccccc45)cc3)c3cccc4oc5ccccc5c34)cc2)cc1]

Compound subA-1 (10 g, 34.6 mmol), Compound sub8 (11.6 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-8 (13.8 g). (Yield: 68%, MS: [M+H]⁺=588)

### Preparation Example 2-9

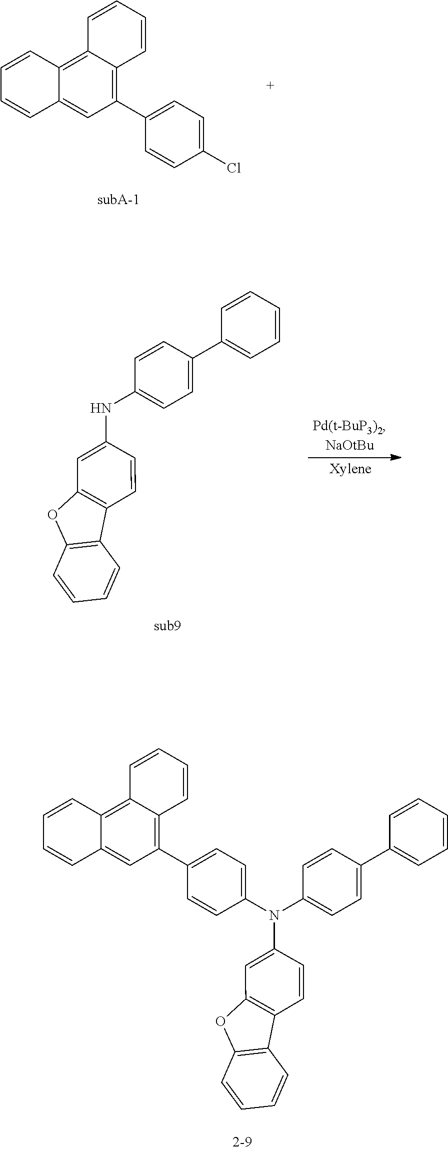 [SMILES: Clc1ccc(-c2cc3ccccc3c3ccccc23)cc1 ; c1ccc(-c2ccc(Nc3ccc4c(c3)oc3ccccc34)cc2)cc1 ; c1ccc(-c2ccc(N(c3ccc(-c4cc5ccccc5c5ccccc45)cc3)c3ccc4c(c3)oc3ccccc34)cc2)cc1]

Compound subA-1 (10 g, 34.6 mmol), Compound sub9 (11.6 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium (0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-9 (10.6 g). (Yield: 52%, MS: [M+H]⁺=588)

### Preparation Example 2-10

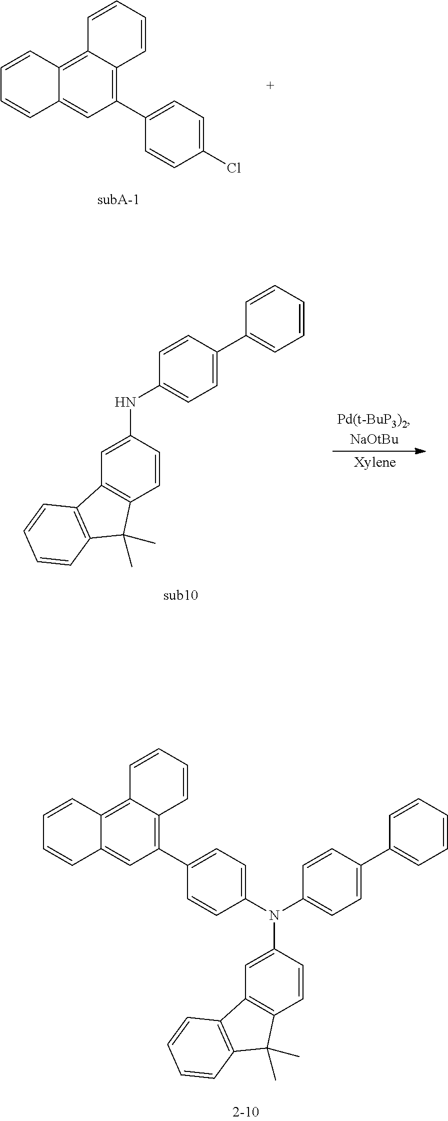 [SMILES: Clc1ccc(-c2cc3ccccc3c3ccccc23)cc1 ; CC1(C)c2ccccc2-c2cc(Nc3ccc(-c4ccccc4)cc3)ccc21 ; CC1(C)c2ccccc2-c2cc(N(c3ccc(-c4ccccc4)cc3)c3ccc(-c4cc5ccccc5c5ccccc45)cc3)ccc21]

Compound subA-1 (10 g, 34.6 mmol), Compound sub10 (12.5 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-10 (11.5 g). (Yield: 54%, MS: [M+H]⁺=614)

### Preparation Example 2-11

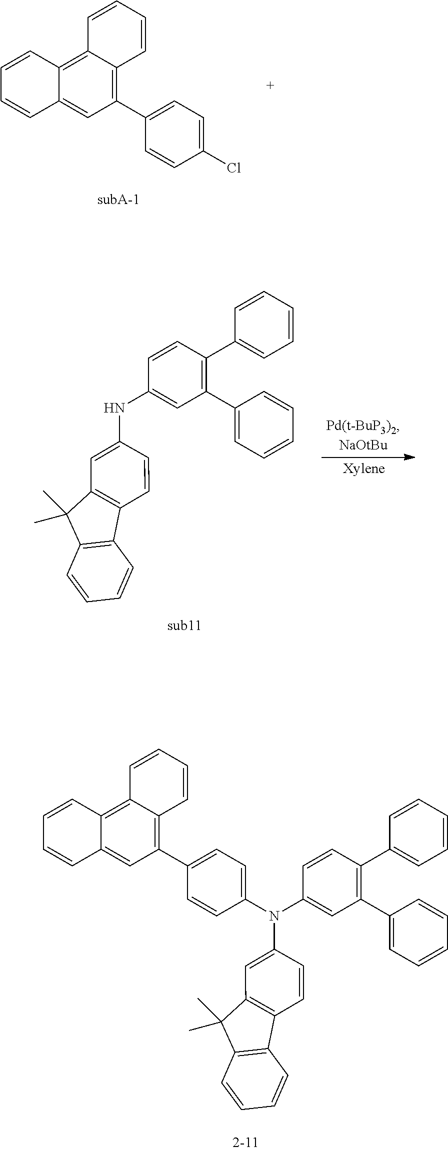 [SMILES: Clc1ccc(-c2cc3ccccc3c3ccccc23)cc1 ; CC1(C)c2ccccc2-c2ccc(Nc3ccc(-c4ccccc4)c(-c4ccccc4)c3)cc21 ; CC1(C)c2ccccc2-c2ccc(N(c3ccc(-c4cc5ccccc5c5ccccc45)cc3)c3ccc(-c4ccccc4)c(-c4ccccc4)c3)cc21]

Compound subA-1 (10 g, 34.6 mmol), Compound sub11 (15.2 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-11 (13.6 g). (Yield: 57%, MS: [M+H]⁺=690)

### Preparation Example 2-12

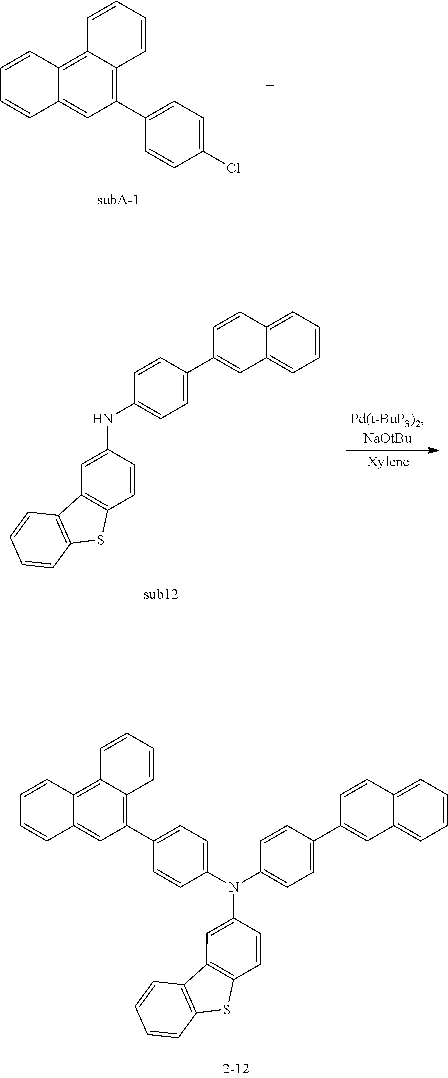 [SMILES: Clc1ccc(-c2cc3ccccc3c3ccccc23)cc1 ; c1ccc2cc(-c3ccc(Nc4ccc5sc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1]

Compound subA-1 (10 g, 34.6 mmol), Compound sub12 (13.9 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-12 (15.8 g). (Yield: 70%, MS: [M+H]⁺=654)

### Preparation Example 2-13

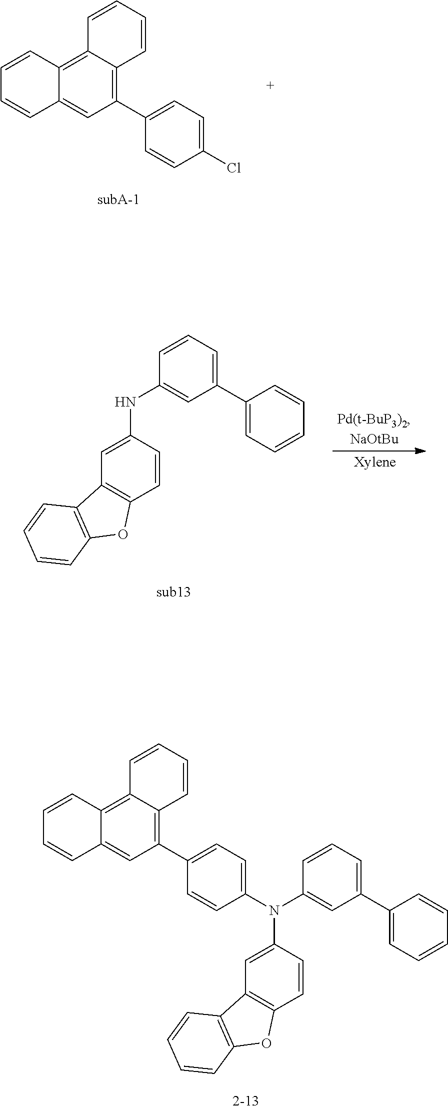 [SMILES: Clc1ccc(-c2cc3ccccc3c3ccccc23)cc1 ; c1ccc(-c2cccc(Nc3ccc4oc5ccccc5c4c3)c2)cc1 ; c1ccc(-c2cccc(N(c3ccc(-c4cc5ccccc5c5ccccc45)cc3)c3ccc4oc5ccccc5c4c3)c2)cc1]

Compound subA-1 (10 g, 34.6 mmol), Compound sub13 (115.5 g, 34.6 mmol) and sodium Cert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-13 (13.8 g). (Yield: 68%, MS: [M+H]⁺=588)

### Preparation Example 2-14

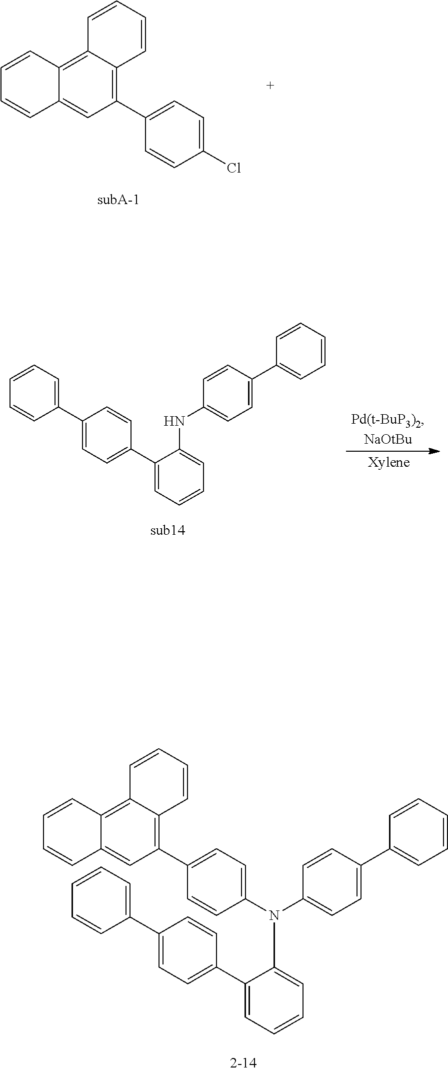 [SMILES: Clc1ccc(-c2cc3ccccc3c3ccccc23)cc1 ; c1ccc(-c2ccc(Nc3ccccc3-c3ccc(-c4ccccc4)cc3)cc2)cc1 ; c1ccc(-c2ccc(-c3ccccc3N(c3ccc(-c4ccccc4)cc3)c3ccc(-c4cc5ccccc5c5ccccc45)cc3)cc2)cc1]

Compound subA-1 (10 g, 34.6 mmol), Compound sub14 (13.8 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-14 (14.8 g). (Yield: 66%, MS: [M+H]⁺=650)

### Preparation Example 2-15

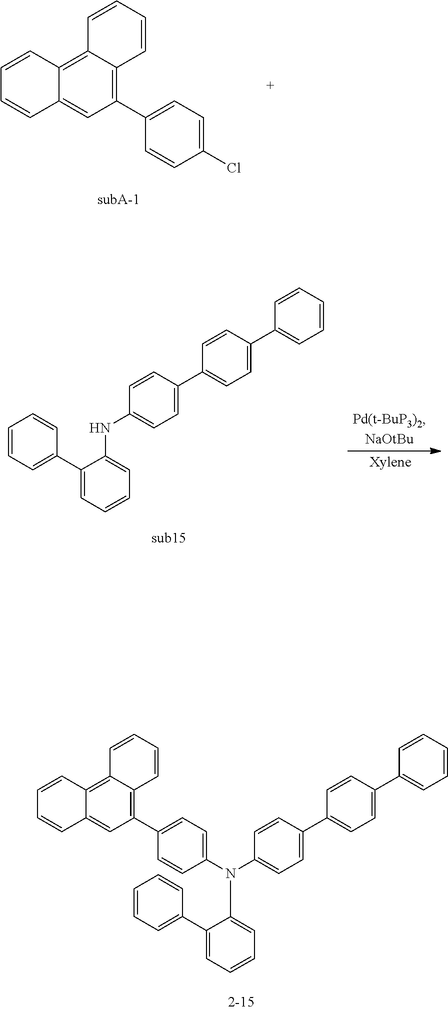 [SMILES: Clc1ccc(-c2cc3ccccc3c3ccccc23)cc1 ; c1ccc(-c2ccc(-c3ccc(Nc4ccccc4-c4ccccc4)cc3)cc2)cc1 ; c1ccc(-c2ccc(-c3ccc(N(c4ccc(-c5cc6ccccc6c6ccccc56)cc4)c4ccccc4-c4ccccc4)cc3)cc2)cc1]

Compound subA-1 (10 g, 34.6 mmol), Compound sub15 (13.8 g, 34.6 mmol) and sodium tert-butoxide (6.7 g, 69.3 mmol) were added to xylene (200 mi) under a nitrogen atmosphere, and the mixture was stirred and refluxed. Then, bis(tri-tert-butylphosphine)palladium (0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-15 (14.4 g). (Yield: 64%, MS: [M+H]⁺=650)

### Preparation Example 2-16

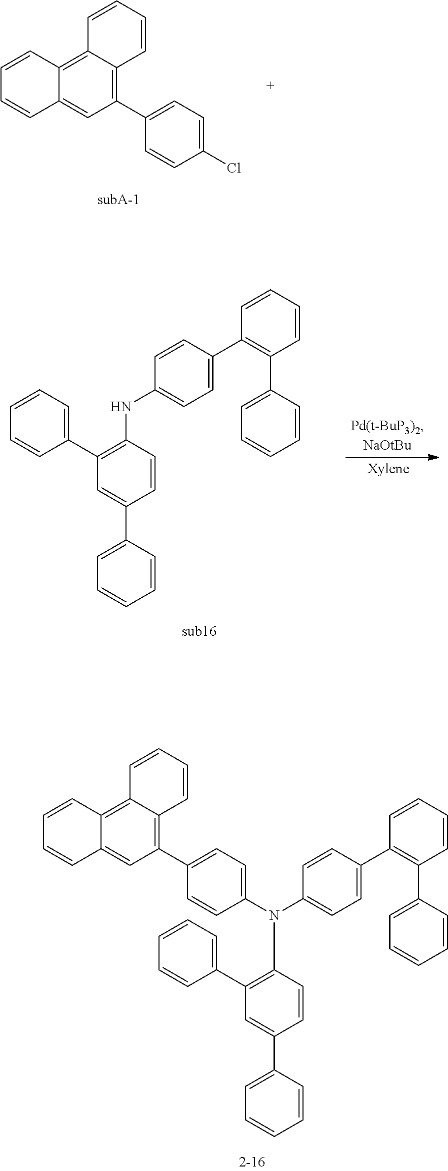 [SMILES: Clc1ccc(-c2cc3ccccc3c3ccccc23)cc1 ; c1ccc(-c2ccc(Nc3ccc(-c4ccccc4-c4ccccc4)cc3)c(-c3ccccc3)c2)cc1 ; c1ccc(-c2ccc(N(c3ccc(-c4ccccc4-c4ccccc4)cc3)c3ccc(-c4cc5ccccc5c5ccccc45)cc3)c(-c3ccccc3)c2)cc1]

Compound subA-1 (10 g, 34.6 mmol), Compound sub16 (16.4 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-16 (13.1 g). (Yield: 52%, MS: [M+H]⁺=726)

### Preparation Example 2-17

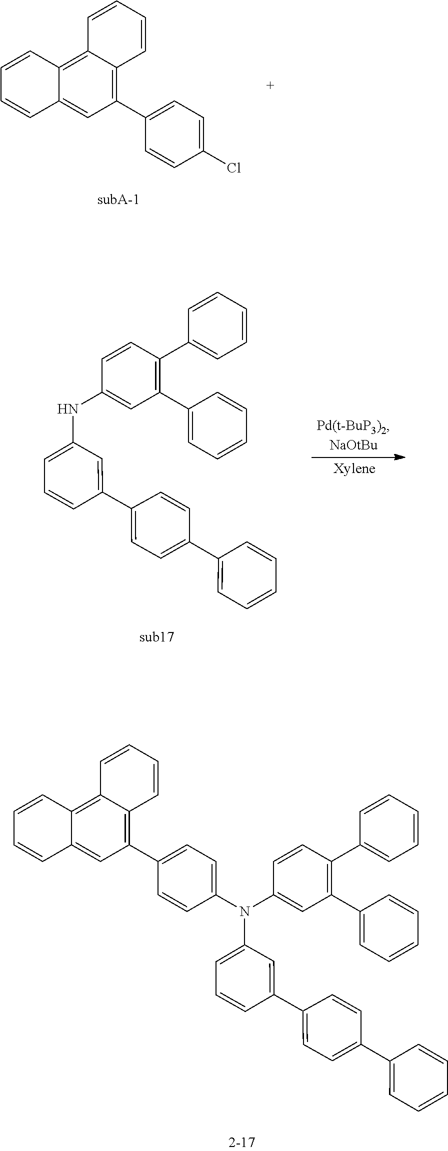 [SMILES: Clc1ccc(-c2cc3ccccc3c3ccccc23)cc1 ; c1ccc(-c2ccc(-c3cccc(Nc4ccc(-c5ccccc5)c(-c5ccccc5)c4)c3)cc2)cc1 ; c1ccc(-c2ccc(-c3cccc(N(c4ccc(-c5cc6ccccc6c6ccccc56)cc4)c4ccc(-c5ccccc5)c(-c5ccccc5)c4)c3)cc2)cc1]

Compound subA-1 (10 g, 34.6 mmol), Compound sub17 (16.4 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-17 (16.6 g). (Yield: 66%, MS: [M+H]⁺=726)

### Preparation Example 2-18

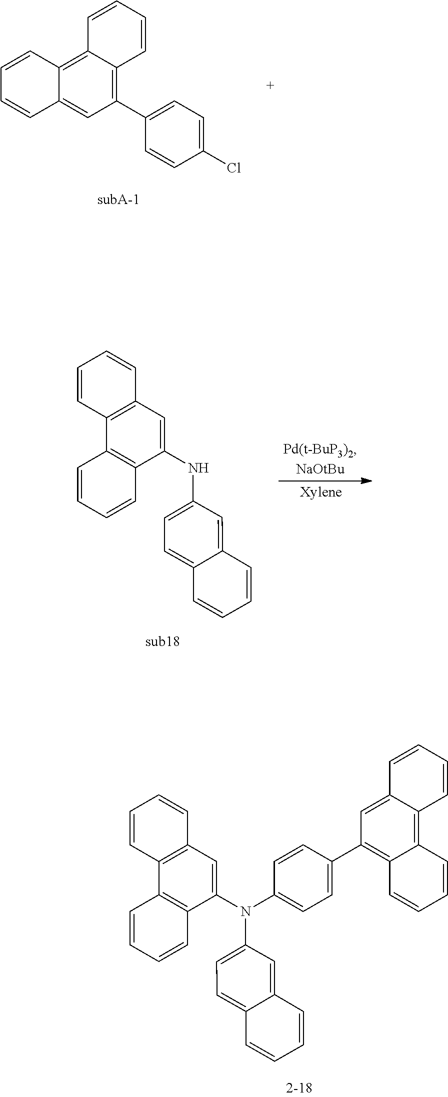 [SMILES: Clc1ccc(-c2cc3ccccc3c3ccccc23)cc1 ; c1ccc2cc(Nc3cc4ccccc4c4ccccc34)ccc2c1 ; c1ccc2cc(N(c3ccc(-c4cc5ccccc5c5ccccc45)cc3)c3cc4ccccc4c4ccccc34)ccc2c1]

Compound subA-1 (10 g, 34.6 mmol), Compound sub18 (11.1 g, 34.6 mmol) and sodium Cert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-18 (11.1 g). (Yield: 56%, MS: [M+H]⁺=572)

### Preparation Example 2-19

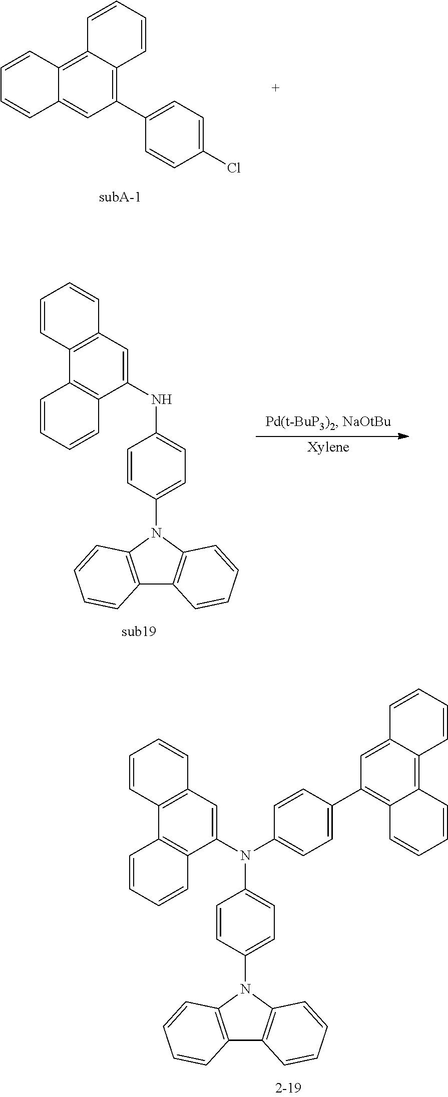

Compound subA-1 (10 g, 34.6 mmol), Compound sub19 (15 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-19 (16.4 g). (Yield: 69%, MS: [M+H]⁺=687)

### Preparation Example 2-20

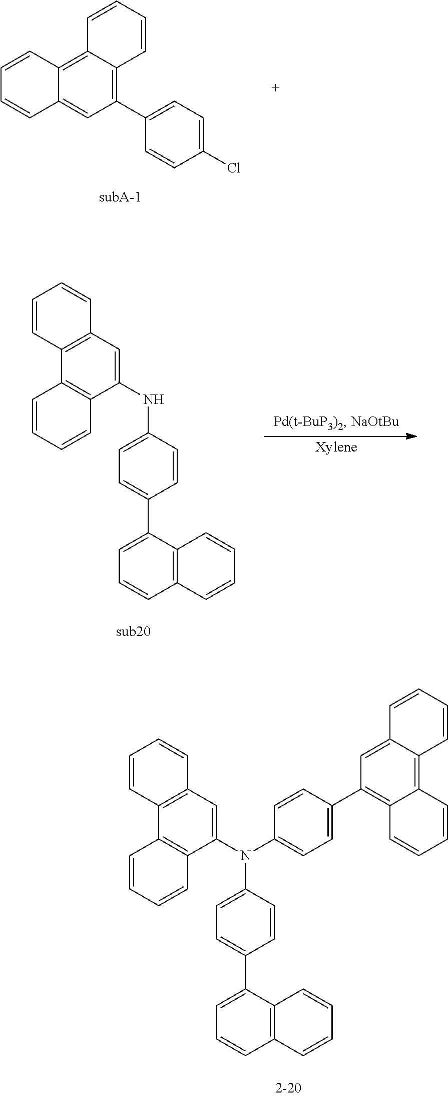

Compound subA-1 (10 g, 34.6 mmol), Compound sub20 (13.7 g, 34.6 mmol) and sodium Cert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-20 (15 g). (Yield: 67%, MS: [M+H]⁺=648)

### Preparation Example 2-21

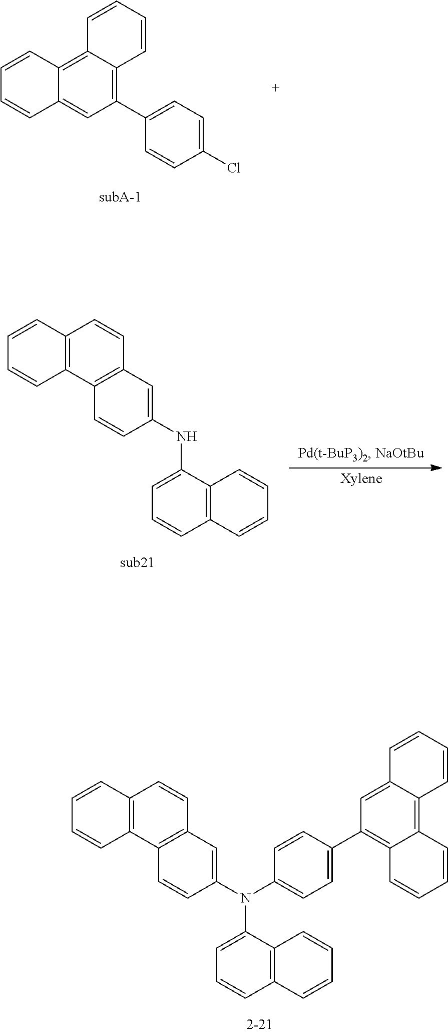

Compound subA-1 (10 g, 34.6 mmol Compound sub21 (11.1 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-21 (10.5 g). (Yield: 53%, MS: [M+H]⁺=572)

### Preparation Example 2-22

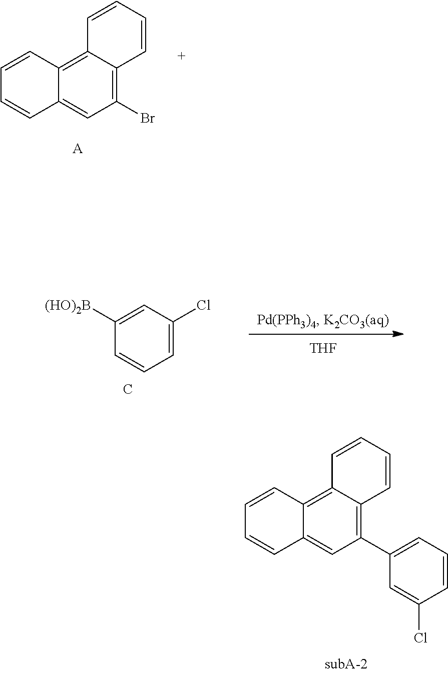

Compound A (15 g, 58.3 mmol) and Compound C (10 g, 64.2 mmol) were added to THF (300 ml) under a nitrogen atmosphere, and the mixture was stirred and refluxed. Then, potassium carbonate (24.2 g, 175 mmol) was dissolved in water (73 ml), added thereto, and the mixture was sufficiently stirred and then tetrakis(triphenylphosphine)palladium (0) (0.7 g, 0.6 mmol) was added. After the reaction for 8 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subA-2 (12.4 g). (Yield: 74%, MS: [M+H]⁺=289)

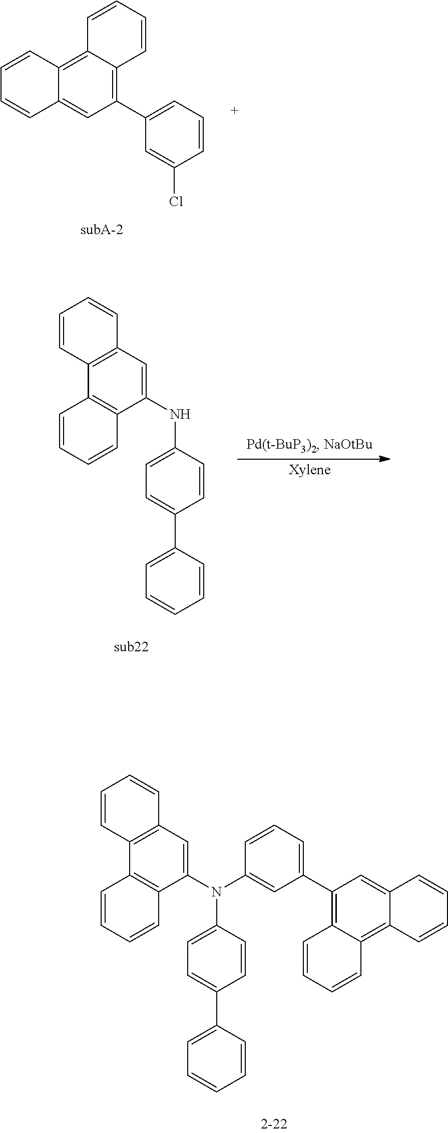

Compound subA-2 (10 g, 34.6 mmol), Compound sub22 (12 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium (0) (0.2 g, 0.3 mmol) was added thereto.

When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-22 (14.1 g). (Yield: 68%, MS: [M+H]⁺=598)

### Preparation Example 2-23

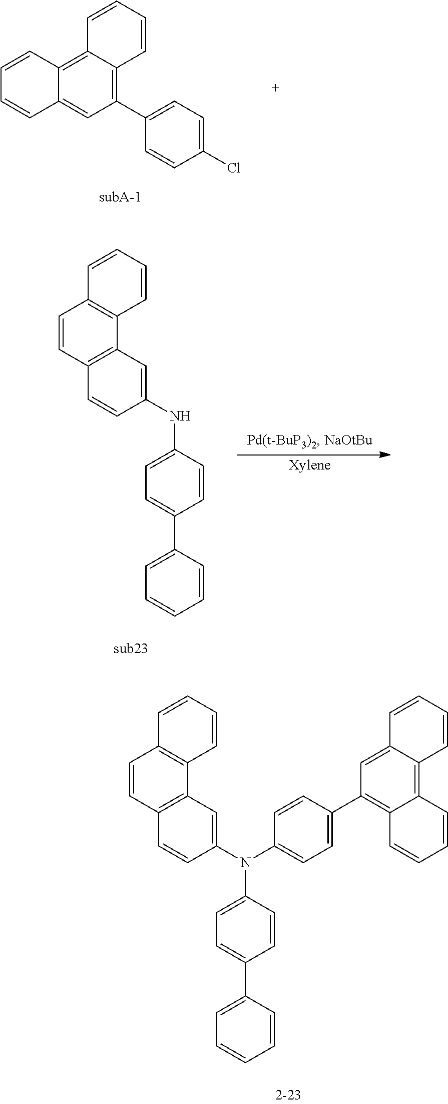

Compound subA-1 (10 g, 34.6 mmol), Compound sub23 (12 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-23 (11 g). (Yield: 53%, MS: [M+H]⁺=598)

### Preparation Example 2-24

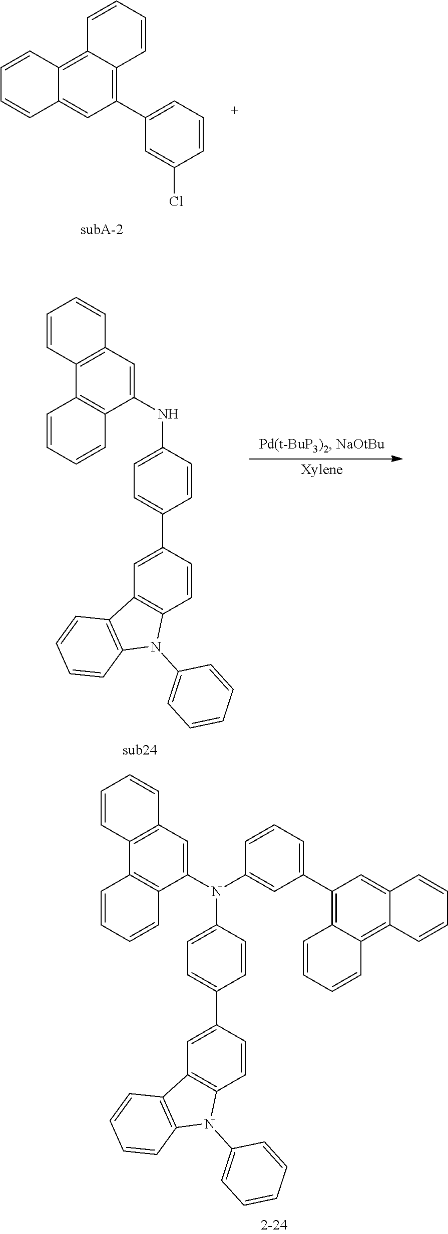

Compound subA-2 (10 g, 34.6 mmol), Compound sub24 (17.7 g, 34.6 mmol) and sodium tert-butoxide (6.7 g, 69.3 mmol) were added to xylene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-24 (15.3 g). (Yield: 58%, MS: [M+H]⁺=763)

### Preparation Example 2-25

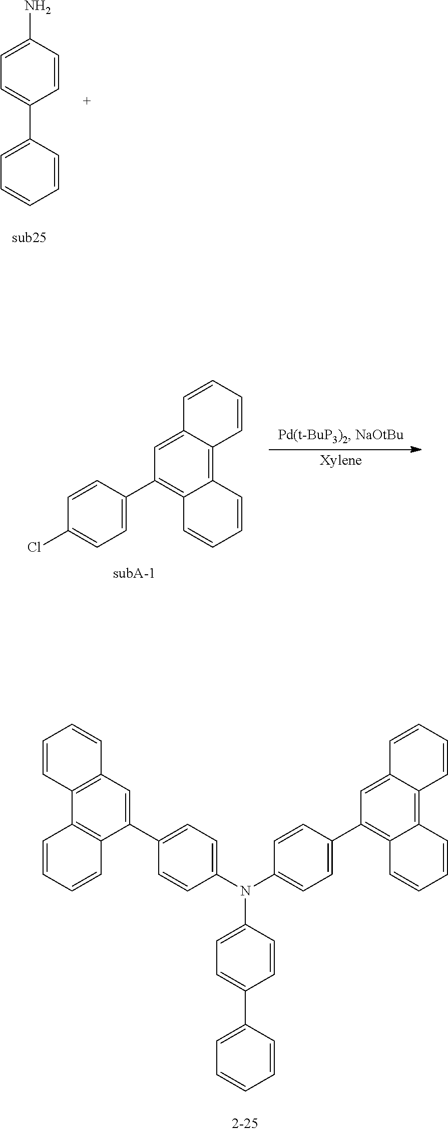

Compound sub25 (10 g, 59.1 mmol), Compound subA-1 (34.1 g, 118.2 mmol) and sodium tert-butoxide (17 g, 177.3 mmol) were added to xylene (200 ml) under a nitrogen atmosphere, and the mixture was stirred and refluxed. Then, bis(tri-tert-butylphosphine)palladium (0) (0.6 g, 1.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-25 (27.1 g). (Yield: 68%, MS: [M+H]⁺=674)

### Preparation Example 2-26

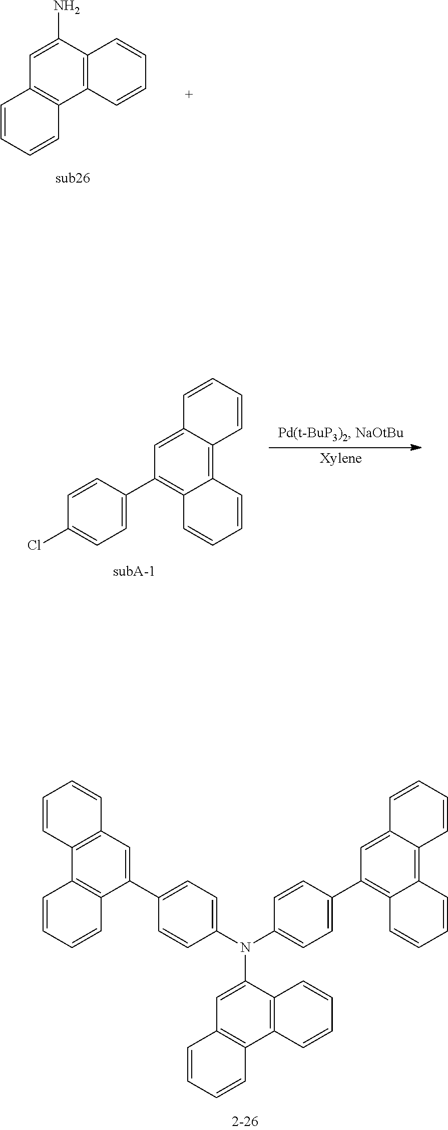

Compound sub26 (10 g, 51.7 mmol), Compound subA-1 (29.9 g, 103.5 mmol) and sodium tert-butoxide (14.9 g, 155.2 mmol) were added to xylene (200 ml) under a nitrogen atmosphere, and the mixture was stirred and refluxed. Then, bis(tri-tert-butylphosphine)palladium(0) (0.5 g, 1 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-26 (18 g). (Yield: 50%, MS: [M+H]⁺=698)

### Preparation Example 2-27

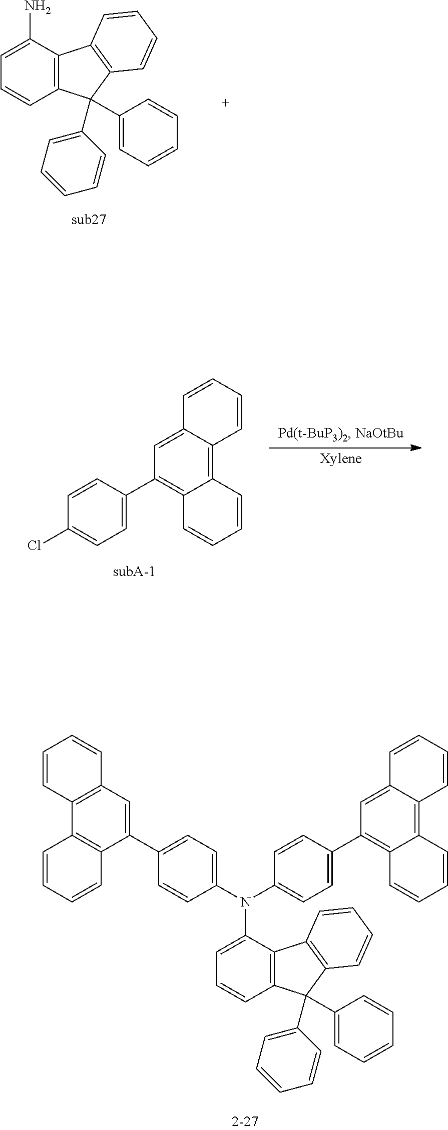

Compound sub27 (10 g, 30 mmol), Compound subA-1 (17.3 g, 60 mmol) and sodium tert-butoxide (8.6 g, 90 mmol) were added to xylene (200 ml) under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-27 (14.6 g). (Yield: 58%, MS: [M+H]⁺=838)

### Preparation Example 2-28

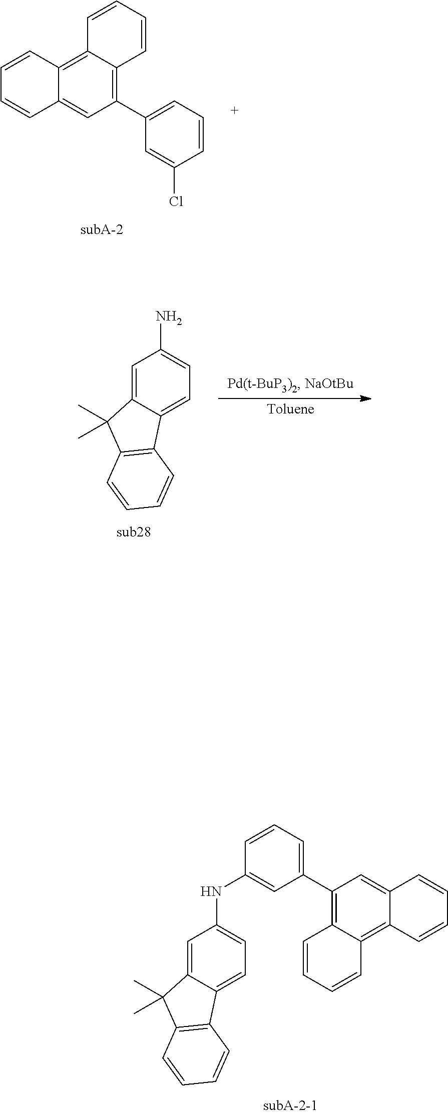

Compound subA-2 (10 g, 34.6 mmol), Compound sub28 (7.2 g, 34.6 mmol) and sodium tert-butoxide (6.7 g, 69.3 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound subA-2-1 (11.2 g). (Yield: 70%, MS: [M+H]⁺=462)

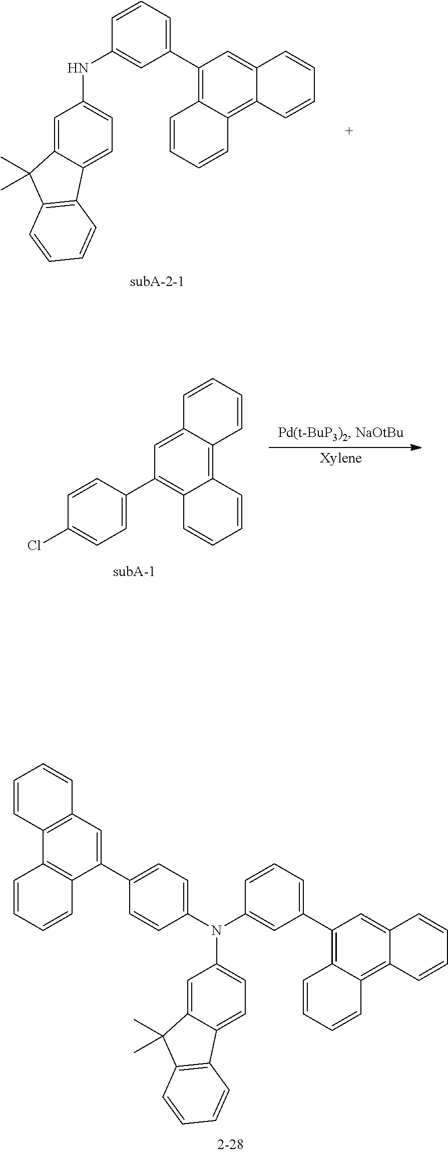

Compound subA-2-1 (10 g, 21.7 mmol), Compound subA-1 (6.3 g, 21.7 mmol) and sodium tert-butoxide (4.2 g, 43.3 mmol) were added to xylene (200 ml) under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-28 (10.7 g). (Yield: 69%, MS: [M+H]⁺=714)

### Preparation Example 2-29

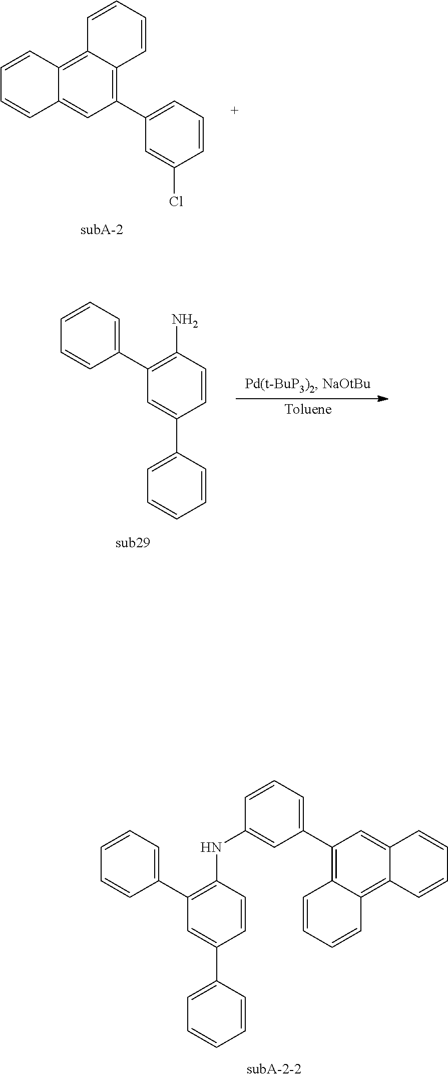

Compound subA-2 (10 g, 34.6 mmol), Compound sub29 (8.5 g, 34.6 mmol) and sodium tert-butoxide (6.7 g, 69.3 mmol) were added to toluene (200 ml) under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound subA-2-2 (9.8 g). (Yield: 57%, MS: [M+H]⁺=498)

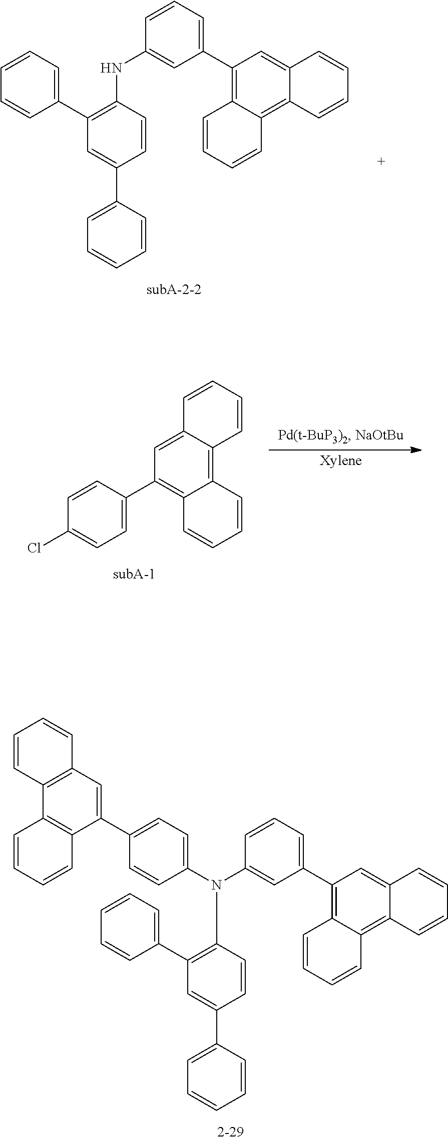

Compound subA-2-2 (10 g, 20.1 mmol), Compound subA-1 (5.8 g, 20.1 mmol) and sodium tert-butoxide (3.9 g, 40.2 mmol) were added to xylene (200 ml) under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-29 (10.1 g). (Yield: 67%, MS: [M+H]⁺=750)

### Preparation Example 2-30

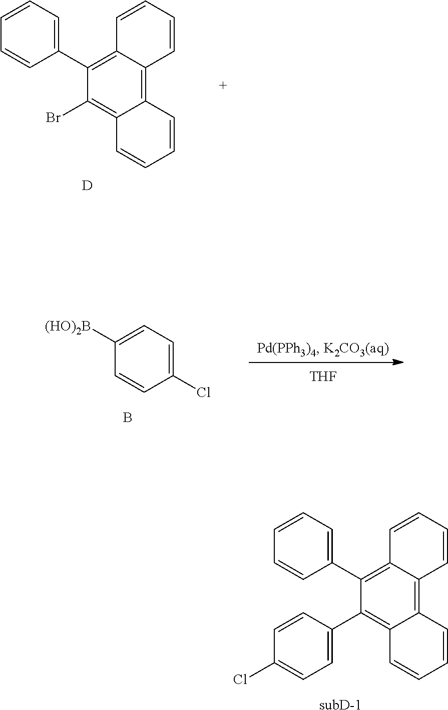

Compound D (15 g, 45 mmol) and Compound B (7.7 g, 49.5 mmol) were added to THF (300 ml) under a nitrogen atmosphere, and the mixture was stirred and refluxed. Then, potassium carbonate (18.7 g, 135 mmol) was dissolved in water (56 ml), added thereto, and the mixture was sufficiently stirred and then tetrakis(triphenylphosphine)palladium(0) (0.5 g, 0.5 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subD-1 (13.1 g). (Yield: 80%, MS: [M+H]⁺=365)

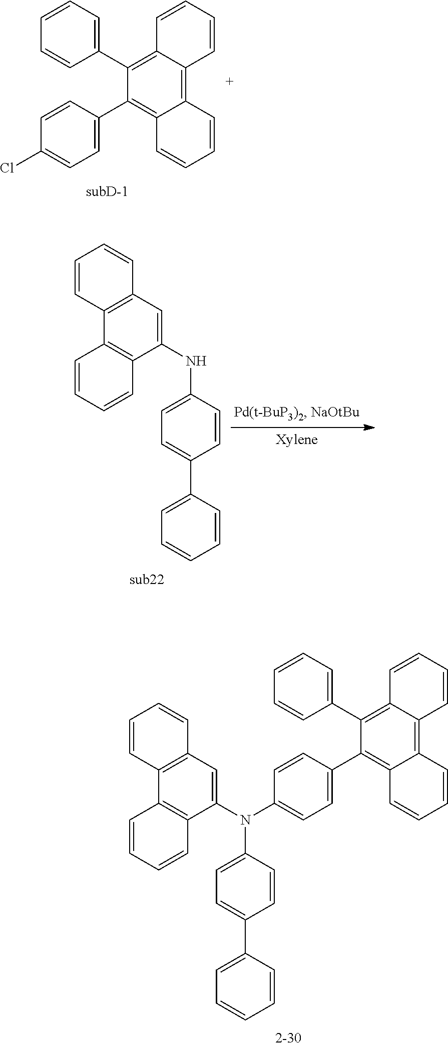 [SMILES: Clc1ccc(-c2c(-c3ccccc3)c3ccccc3c3ccccc23)cc1 ; c1ccc(-c2ccc(Nc3cc4ccccc4c4ccccc34)cc2)cc1 ; c1ccc(-c2ccc(N(c3ccc(-c4c(-c5ccccc5)c5ccccc5c5ccccc45)cc3)c3cc4ccccc4c4ccccc34)cc2)cc1]

Compound subD-1 (10 g, 27.4 mmol), Compound sub22 (9.5 g, 27.4 mmol) and sodium tert-butoxide (5.3 g, 54.8 mmol) were added to xylene (200 ml) under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-30 (9.6 g). (Yield: 52%, MS: [M+H]⁺=674)

### Preparation Example 2-31

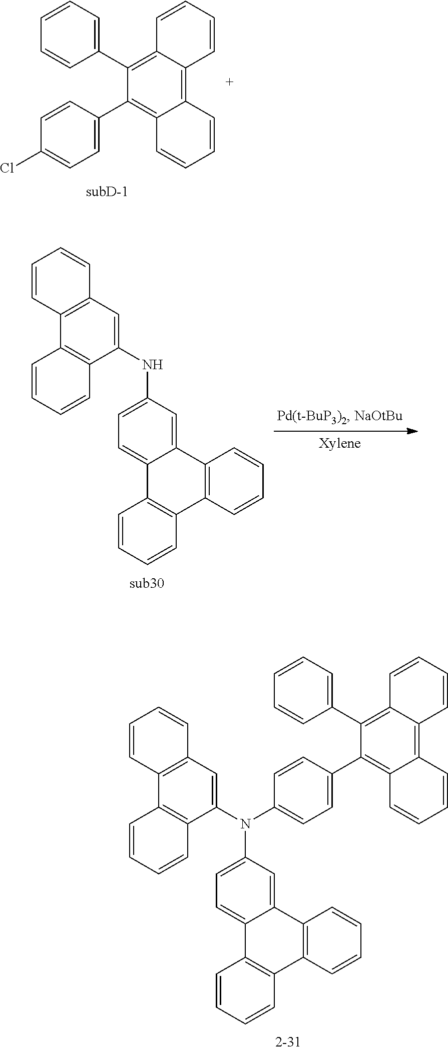 [SMILES: Clc1ccc(-c2c(-c3ccccc3)c3ccccc3c3ccccc23)cc1 ; c1ccc2c(c1)cc(Nc1ccc3c4ccccc4c4ccccc4c3c1)c1ccccc12 ; c1ccc(-c2c(-c3ccc(N(c4ccc5c6ccccc6c6ccccc6c5c4)c4cc5ccccc5c5ccccc45)cc3)c3ccccc3c3ccccc23)cc1]

Compound subD-1 (10 g, 27.4 mmol), Compound sub30 (11.5 g, 27.4 mmol) and sodium tert-butoxide (5.3 g, 54.8 mmol) were added to xylene (200 ml) under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-31 (13.9 g). (Yield: 68%, MS: [M+H]⁺=748)

### Preparation Example 2-32

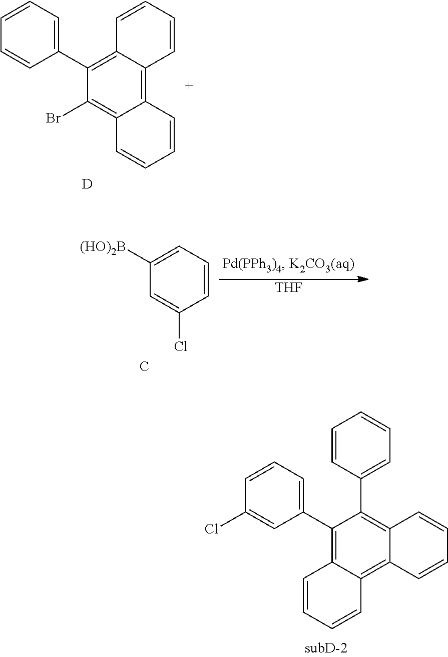

Compound D (15 g, 45 mmol) and Compound C (7.7 g, 49.5 mmol) were added to THF (300 ml) under a nitrogen atmosphere, and the mixture was stirred and refluxed. Then, potassium carbonate (18.7 g, 135 mmol) was dissolved in water (56 ml), added thereto, and the mixture was sufficiently stirred and then tetrakis(triphenylphosphine)palladium(0) (0.5 g, 0.5 mmol) was added. After the reaction for 9 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subD-2 (9.3 g). (Yield: 72%, MS: [M+H]⁺=289)

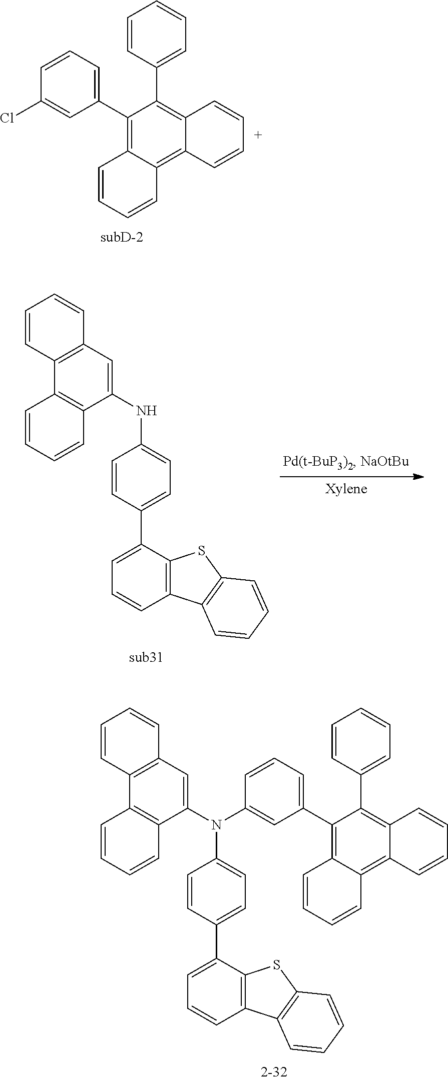 [SMILES: Clc1cccc(-c2c(-c3ccccc3)c3ccccc3c3ccccc23)c1 ; c1ccc2c(c1)cc(Nc1ccc(-c3cccc4c3sc3ccccc34)cc1)c1ccccc12 ; c1ccc(-c2c(-c3cccc(N(c4ccc(-c5cccc6c5sc5ccccc56)cc4)c4cc5ccccc5c5ccccc45)c3)c3ccccc3c3ccccc23)cc1]

Compound subD-2 (10 g, 27.4 mmol), Compound sub31 (12.4 g, 27.4 mmol) and sodium Cert-butoxide (5.3 g, 54.8 mmol) were added to xylene (200 ml) under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-32 (15 g). (Yield: 70%, MS: [M+H]⁺=780)

### Preparation Example 2-33

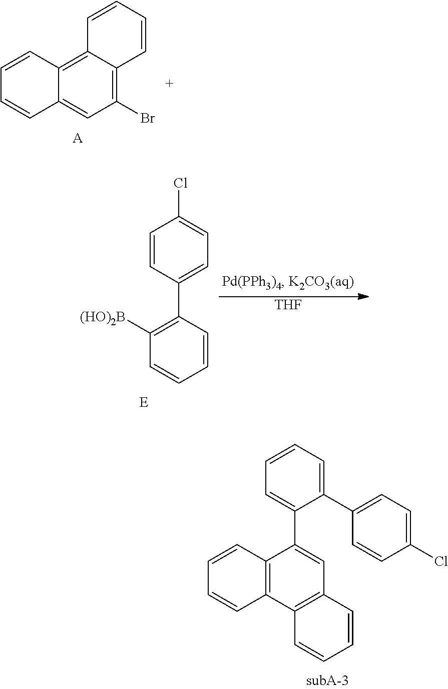

Compound A (15 g, 58.3 mmol) and Compound E (14.9 g, 64.2 mmol) were added to THF (300 ml) under a nitrogen atmosphere, and the mixture was stirred and refluxed. Then, potassium carbonate (24.2 g, 175 mmol) was dissolved in water (73 ml), added thereto, and the mixture was sufficiently stirred and then tetrakis(triphenylphosphine)palladium(0) (0.7 g, 0.6 mmol) was added. After the reaction for 10 hours, the reaction mixture was cooled to room temperature, and the organic layer and the aqueous layer were separated, and then the organic layer was distilled. This was again dissolved in chloroform, washed twice with water, and then the organic layer was separated, anhydrous magnesium sulfate was added thereto, the mixture was stirred and filtered, and the filtrate was distilled under reduced pressure. The concentrated compound was purified by silica gel column chromatography to give Compound subA-3 (14.9 g). (Yield: 70%, MS: [M+H]⁺=365)

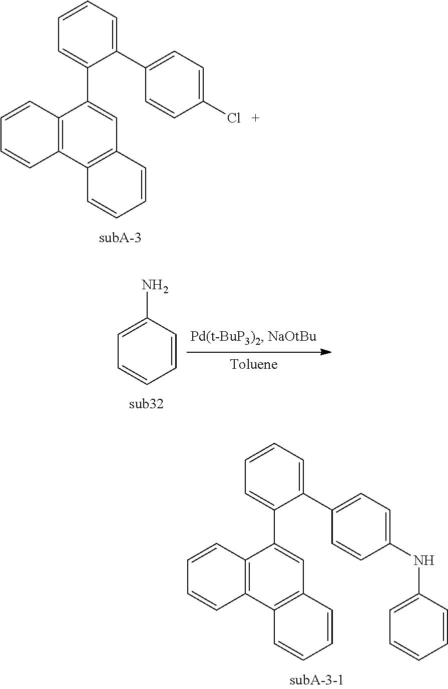 [SMILES: Clc1ccc(-c2ccccc2-c2cc3ccccc3c3ccccc23)cc1 ; Nc1ccccc1 ; c1ccc(Nc2ccc(-c3ccccc3-c3cc4ccccc4c4ccccc34)cc2)cc1]

Compound subA-3 (10 g, 27.4 mmol), Compound sub32 (2.6 g, 27.4 mmol) and sodium tert-butoxide (5.3 g, 54.8 mmol) were added to toluene (200 ml) under a nitrogen atmosphere, and the mixture was stirred and refluxed. Then, bis(tri-tert-butylphosphine)palladium(0) (0.1 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound subA-3-1 (5.8 g). (Yield: 50%, MS: [M+H]⁺=422)

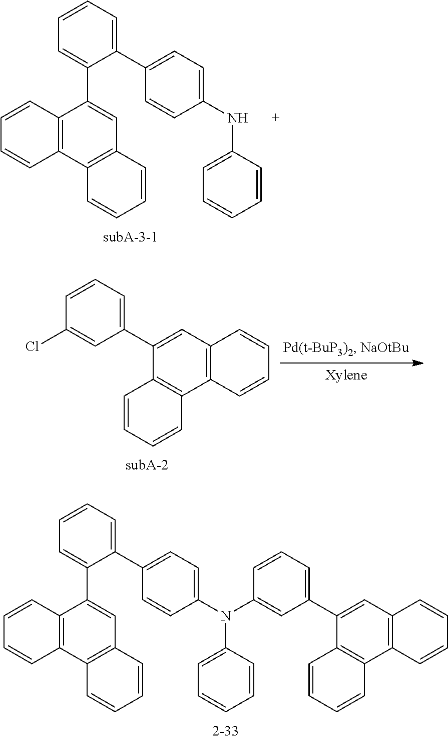 [SMILES: c1ccc(Nc2ccc(-c3ccccc3-c3cc4ccccc4c4ccccc34)cc2)cc1 ; Clc1cccc(-c2cc3ccccc3c3ccccc23)c1 ; c1ccc(N(c2ccc(-c3ccccc3-c3cc4ccccc4c4ccccc34)cc2)c2cccc(-c3cc4ccccc4c4ccccc34)c2)cc1]

Compound subA-3-1 (10 g, 23.7 mmol), Compound subA-2 (6.9 g, 23.7 mmol) and sodium tert-butoxide (4.6 g, 47.4 mmol) were added to xylene (200 ml) under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-33 (8.9 g). (Yield: 56%, MS: [M+H]⁺=674)

### Preparation Example 2-34

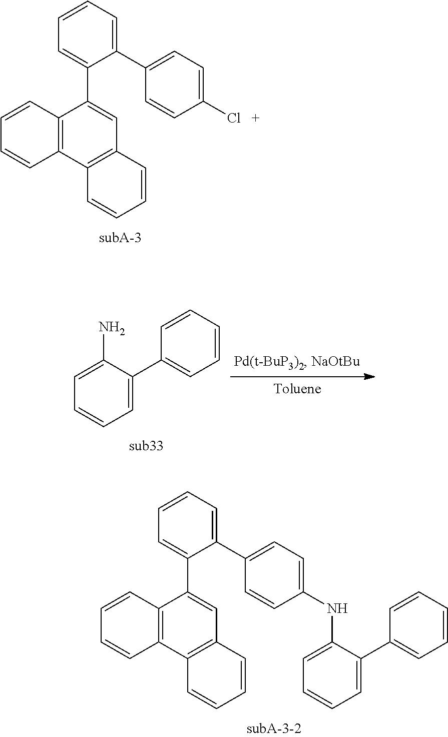 [SMILES: Clc1ccc(-c2ccccc2-c2cc3ccccc3c3ccccc23)cc1 ; Nc1ccccc1-c1ccccc1 ; c1ccc(-c2ccccc2Nc2ccc(-c3ccccc3-c3cc4ccccc4c4ccccc34)cc2)cc1]

Compound subA-3 (10 g, 27.4 mmol), Compound sub33 (4.6 g, 27.4 mmol) and sodium tert-butoxide (5.3 g, 54.8 mmol) were added to toluene (200 ml) under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound subA-3-2 (9.1 g). (Yield: 67%, MS: [M+H]⁺=498)

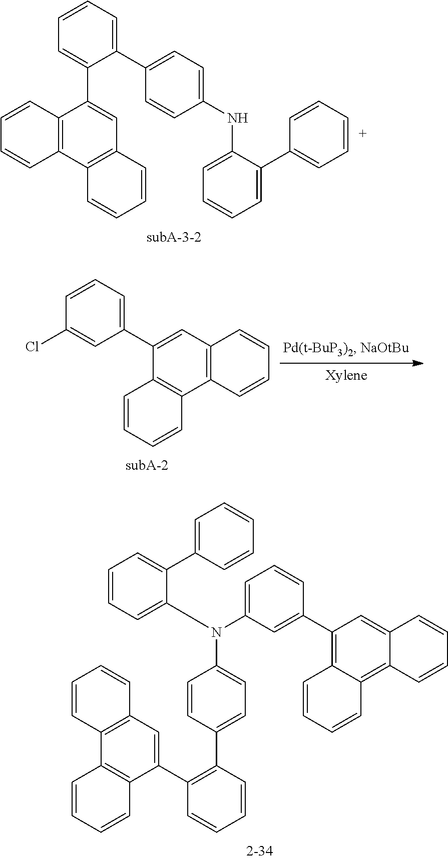 [SMILES: c1ccc(-c2ccccc2Nc2ccc(-c3ccccc3-c3cc4ccccc4c4ccccc34)cc2)cc1 ; Clc1cccc(-c2cc3ccccc3c3ccccc23)c1 ; c1ccc(-c2ccccc2N(c2ccc(-c3ccccc3-c3cc4ccccc4c4ccccc34)cc2)c2cccc(-c3cc4ccccc4c4ccccc34)c2)cc1]

Compound subA-3-2 (10 g, 20.1 mmol), Compound subA-2 (5.8 g, 20.1 mmol) and sodium tert-butoxide (3.9 g, 40.2 mmol) were added to xylene (200 ml) under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-34 (9.6 g). (Yield: 64%, MS: [M+H]⁺=750)

### Preparation Example 2-35

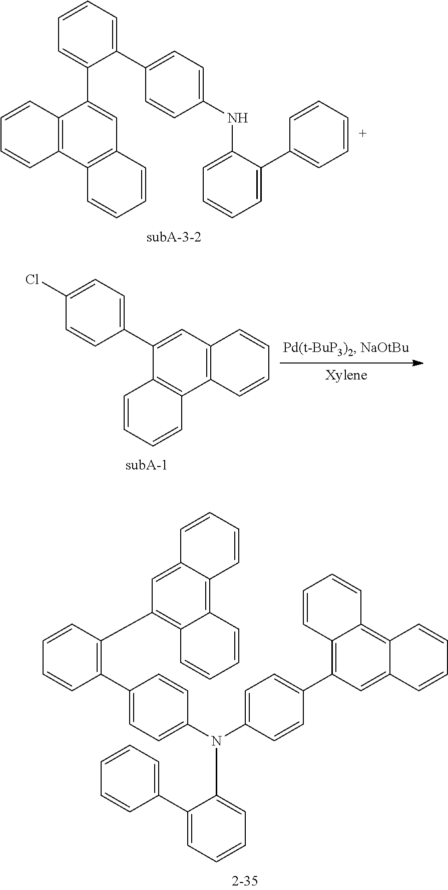 [SMILES: c1ccc(-c2ccccc2Nc2ccc(-c3ccccc3-c3cc4ccccc4c4ccccc34)cc2)cc1 ; Clc1ccc(-c2cc3ccccc3c3ccccc23)cc1 ; c1ccc(-c2ccccc2N(c2ccc(-c3ccccc3-c3cc4ccccc4c4ccccc34)cc2)c2ccc(-c3cc4ccccc4c4ccccc34)cc2)cc1]

Compound subA-3-2 (10 g, 20.1 mmol), Compound subA-1 (5.8 g, 20.1 mmol) and sodium tert-butoxide (3.9 g, 40.2 mmol) were added to xylene (200 ml) under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-35 (8.6 g). (Yield: 57%, MS: [M+H]⁺=750)

### Preparation Example 2-36

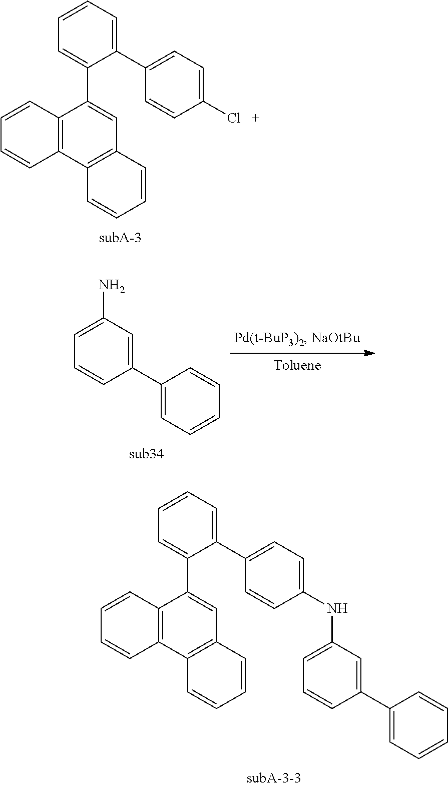 [SMILES: Clc1ccc(-c2ccccc2-c2cc3ccccc3c3ccccc23)cc1 ; Nc1cccc(-c2ccccc2)c1 ; c1ccc(-c2cccc(Nc3ccc(-c4ccccc4-c4cc5ccccc5c5ccccc45)cc3)c2)cc1]

Compound subA-3 (10 g, 27.4 mmol), Compound sub34 (4.6 g, 27.4 mmol) and sodium tert-butoxide (5.3 g, 54.8 mmol) were added to toluene (200 ml) under a nitrogen atmosphere, and the mixture was stirred and refluxed. Then, bis(tri-tert-butylphosphine)palladium(0) (0.1 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound subA-3-3 (8.6 g). (Yield: 63%, MS: [M+H]⁺=498)

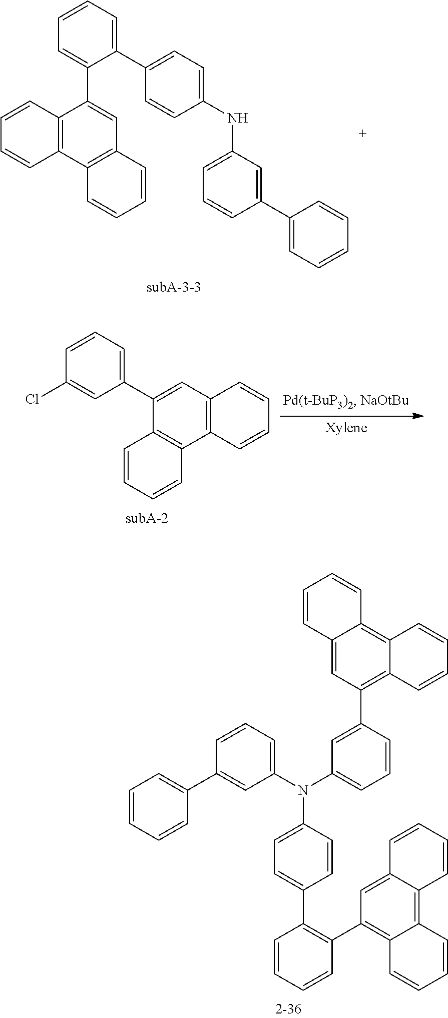 [SMILES: c1ccc(-c2cccc(Nc3ccc(-c4ccccc4-c4cc5ccccc5c5ccccc45)cc3)c2)cc1 ; Clc1cccc(-c2cc3ccccc3c3ccccc23)c1 ; c1ccc(-c2cccc(N(c3ccc(-c4ccccc4-c4cc5ccccc5c5ccccc45)cc3)c3cccc(-c4cc5ccccc5c5ccccc45)c3)c2)cc1]

Compound subA-3-3 (10 g, 20.1 mmol), Compound subA-2 (5.8 g, 20.1 mmol) and sodium tert-butoxide (3.9 g, 40.2 mmol) were added to xylene (200 ml) under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-36 (10.4 g). (Yield: 69%, MS: [M+H]⁺=750)

### Preparation Example 2-37

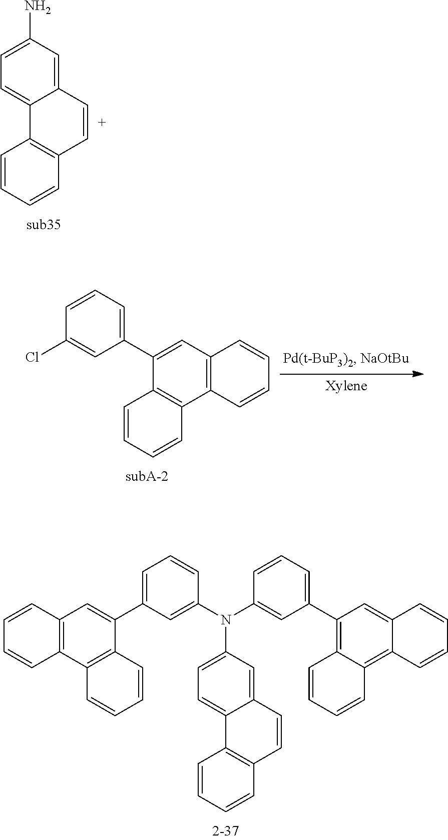 [SMILES: Nc1ccc2c(ccc3ccccc32)c1 ; Clc1cccc(-c2cc3ccccc3c3ccccc23)c1 ; c1cc(-c2cc3ccccc3c3ccccc23)cc(N(c2cccc(-c3cc4ccccc4c4ccccc34)c2)c2ccc3c(ccc4ccccc43)c2)c1]

Compound sub35 (10 g, 51.7 mmol), Compound subA-2 (29.9 g, 103.5 mmol) and sodium tert-butoxide (14.9 g, 155.2 mmol) were added to xylene (200 ml) under a nitrogen atmosphere, and the mixture was stirred and refluxed. Then, bis(tri-tert-butylphosphine)palladium(0) (0.5 g, 1 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-37 (23.8 g). (Yield: 66%, MS: [M+H]⁺=698)

### Preparation Example 2-38

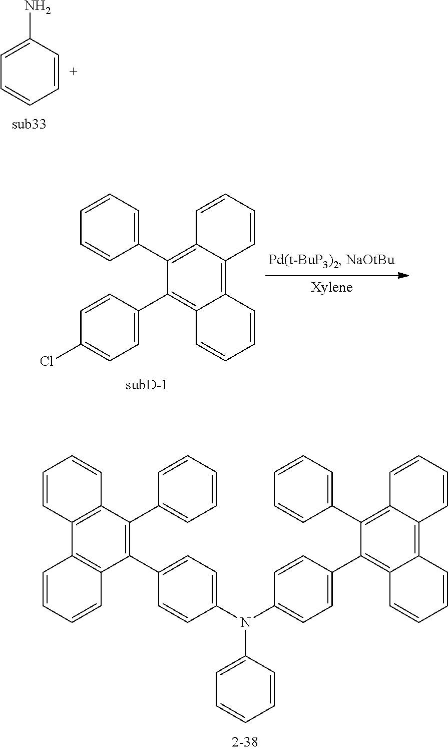 [SMILES: Nc1ccccc1 ; Clc1ccc(-c2c(-c3ccccc3)c3ccccc3c3ccccc23)cc1 ; c1ccc(-c2c(-c3ccc(N(c4ccccc4)c4ccc(-c5c(-c6ccccc6)c6ccccc6c6ccccc56)cc4)cc3)c3ccccc3c3ccccc23)cc1]

Compound sub33 (10 g, 107.4 mmol), Compound subD-1 (78.4 g, 214.8 mmol) and sodium tert-butoxide (31 g, 322.1 mmol) were added to xylene (200 ml) under a nitrogen atmosphere, and the mixture was stirred and refluxed. Then, bis(tri-tert-butylphosphine)palladium(0) (1.1 g, 2.1 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to give Compound 2-38 (53.9 g). (Yield: 67%, MS: [M+H]⁺=750)

### EXAMPLE

### Example 1

A glass substrate on which a thin film of ITO (indium tin oxide) was coated in a thickness of 1,000 Å was put into distilled water containing the detergent dissolved therein and washed by the ultrasonic wave. In this case, the used detergent was a product commercially available from Fisher Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was formed in a thickness of 1150 Å as a hole injection layer, but the following compound A-1 was p-doped at a concentration of 1.5 wt. %. The following compound HT-1 was vacuum deposited on the hole injection layer to form a hole transport layer with a film thickness of 800 Å. Then, the following compound EB-1 was vacuum deposited on the hole transport layer to form an electron blocking layer with a film thickness of 150 Å. Then, the previously prepared Compound 1 and Compound 2-1 as a host, and the following compound Dp-7 as a dopant were respectively vacuum deposited in a weight ratio of 49:49:2 on the electron blocking layer to form a light emitting layer with a film thickness of 400 Å. The following compound HB-1 was vacuum deposited on the light emitting layer to form a hole blocking layer with a film thickness of 30 Å. The following compound ET-1 and the following compound LiQ were vacuum deposited in a ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

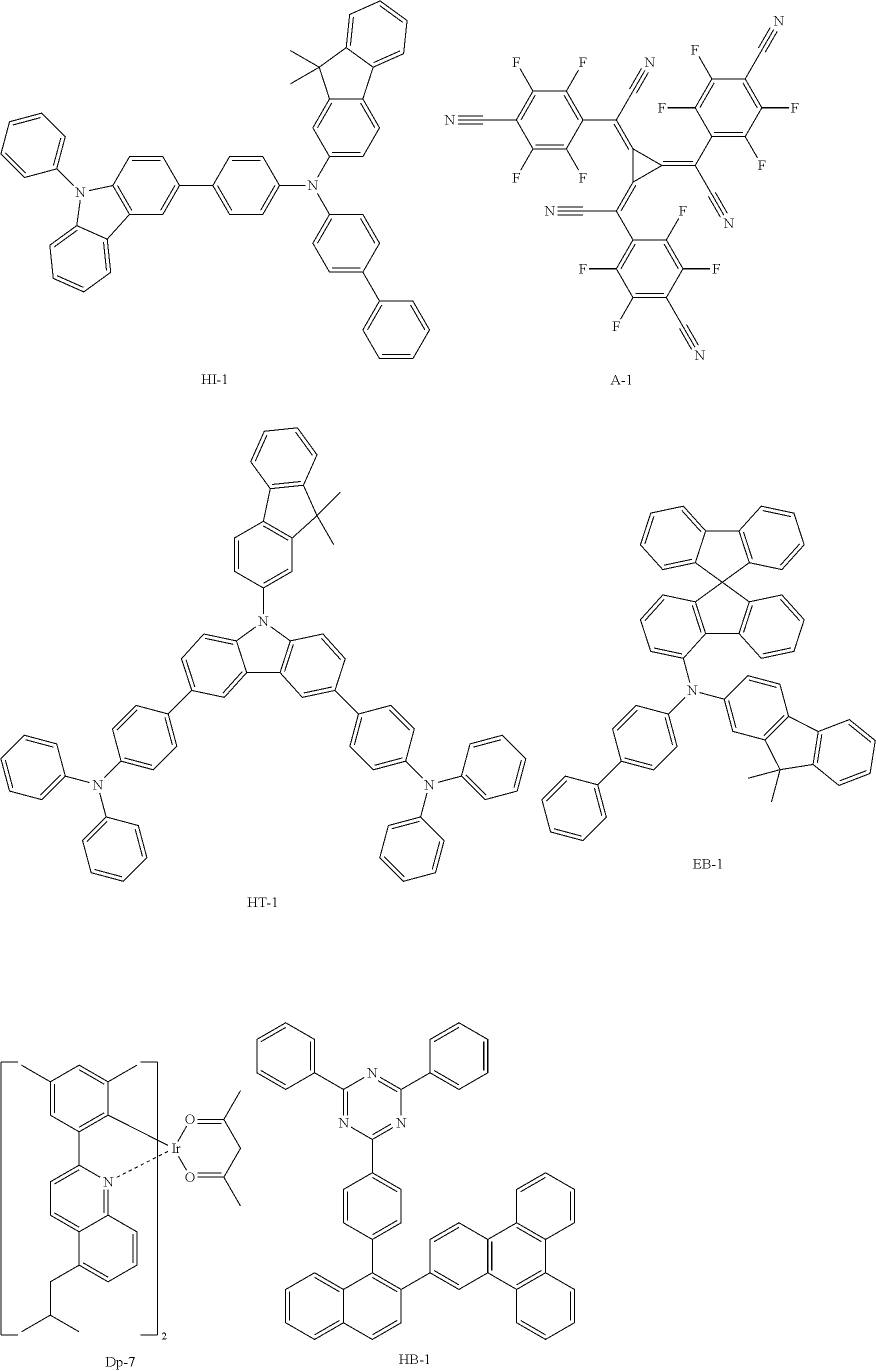

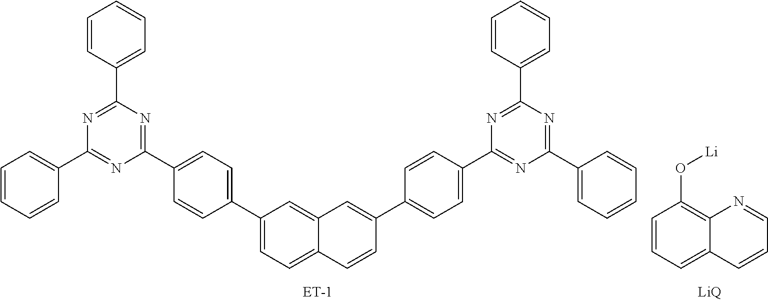

In the above-mentioned processes, the deposition rates of the organic materials were maintained at 0.4 to 0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷ to 5×10⁻⁶ torr, thereby manufacturing an organic light emitting device.

### Examples 2 to 100

The organic light emitting devices were manufactured in the same manner as in Example 1, except that the compounds shown in Tables 1 to 3 below were used as a host of the light emitting layer.

### Comparative Examples 1 to 85

The organic light emitting devices were manufactured in the same manner as in Example 1, except that the compounds shown in Tables 4 to 7 below were used as a host of the light emitting layer. In Tables 6 and 7 below, it means that a single compound was used as the host of the light emitting layer, and the compounds in Table 7 are as follows, respectively.

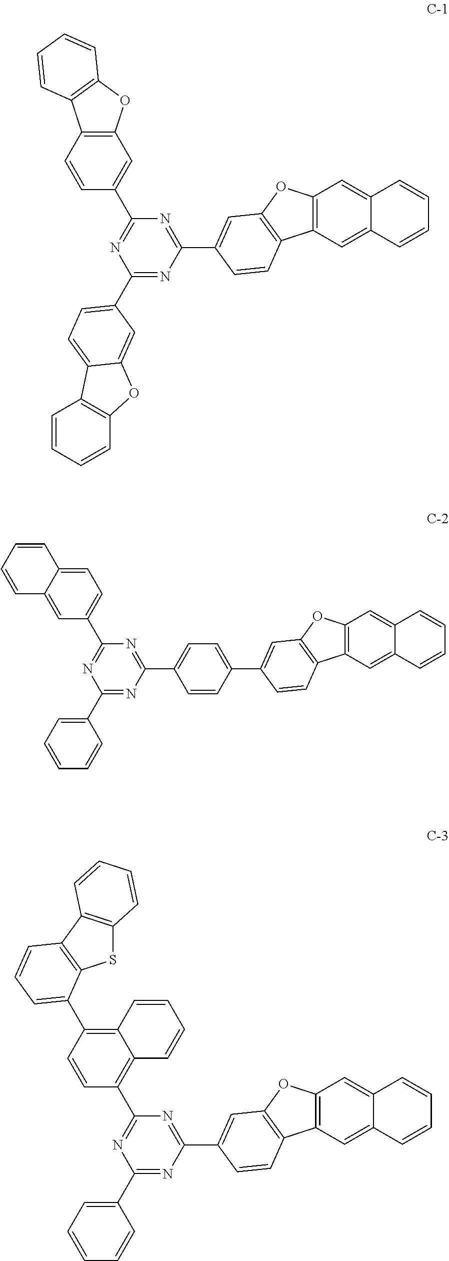 [SMILES: c1ccc2cc3c(cc2c1)oc1cc(-c2nc(-c4ccc5c(c4)oc4ccccc45)nc(-c4ccc5c(c4)oc4ccccc45)n2)ccc13 ; c1ccc(-c2nc(-c3ccc(-c4ccc5c(c4)oc4cc6ccccc6cc45)cc3)nc(-c3ccc4ccccc4c3)n2)cc1 ; c1ccc(-c2nc(-c3ccc4c(c3)oc3cc5ccccc5cc34)nc(-c3ccc(-c4cccc5c4sc4ccccc45)c4ccccc34)n2)cc1]

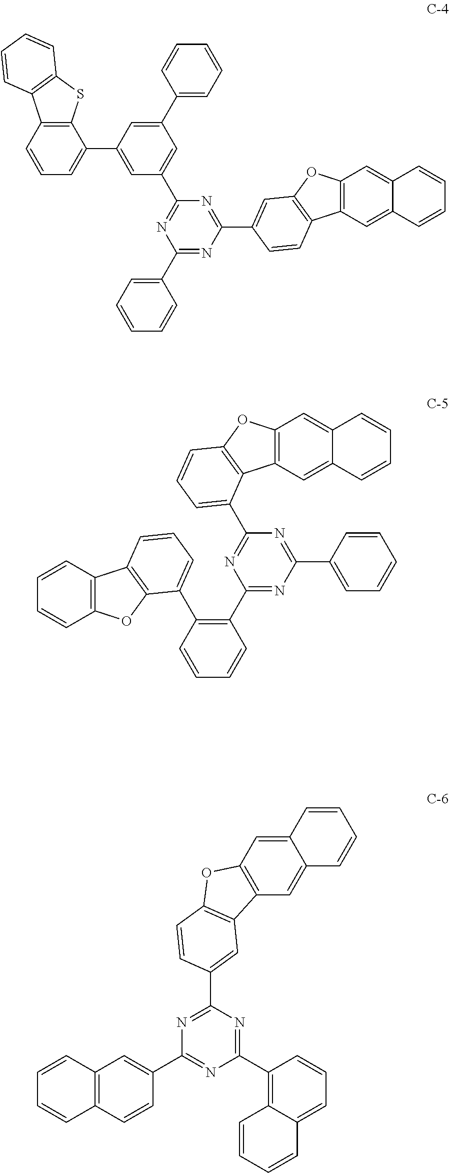 [SMILES: c1ccc(-c2cc(-c3nc(-c4ccccc4)nc(-c4ccc5c(c4)oc4cc6ccccc6cc45)n3)cc(-c3cccc4c3sc3ccccc34)c2)cc1 ; c1ccc(-c2nc(-c3ccccc3-c3cccc4c3oc3ccccc34)nc(-c3cccc4oc5cc6ccccc6cc5c34)n2)cc1 ; c1ccc2cc(-c3nc(-c4ccc5oc6cc7ccccc7cc6c5c4)nc(-c4cccc5ccccc45)n3)ccc2c1]

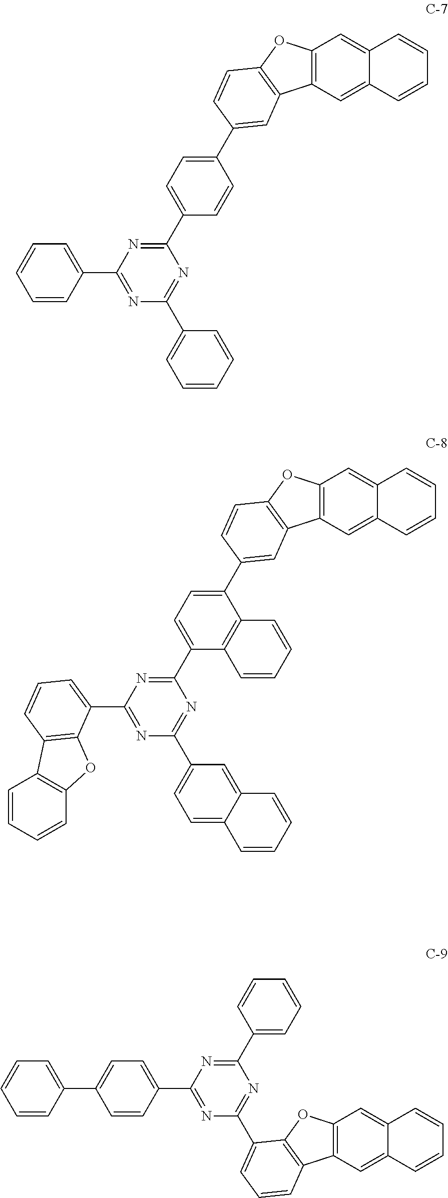 [SMILES: c1ccc(-c2nc(-c3ccccc3)nc(-c3ccc(-c4ccc5oc6cc7ccccc7cc6c5c4)cc3)n2)cc1 ; c1ccc2cc(-c3nc(-c4ccc(-c5ccc6oc7cc8ccccc8cc7c6c5)c5ccccc45)nc(-c4cccc5c4oc4ccccc45)n3)ccc2c1 ; c1ccc(-c2ccc(-c3nc(-c4ccccc4)nc(-c4cccc5c4oc4cc6ccccc6cc45)n3)cc2)cc1]

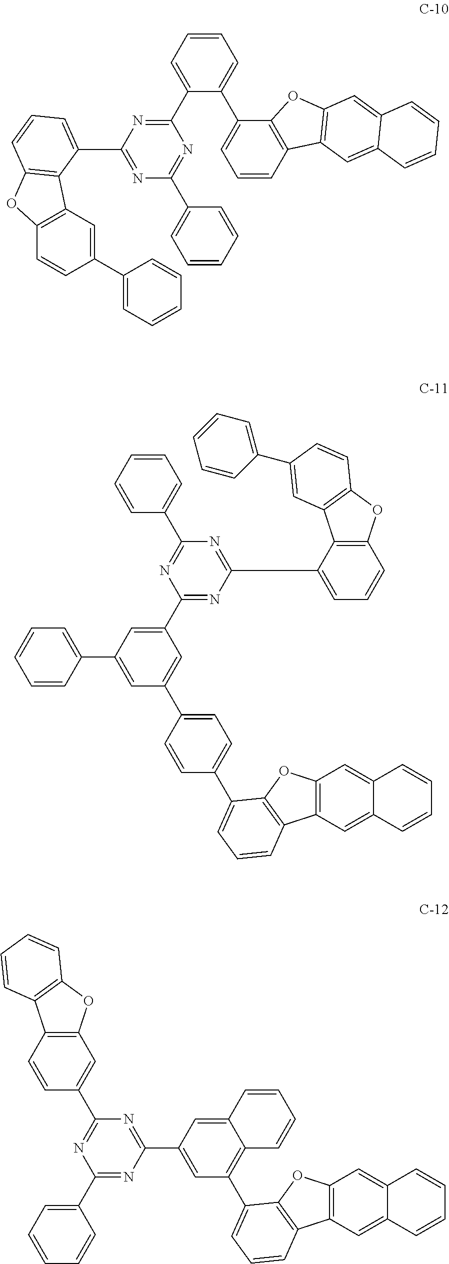 [SMILES: c1ccc(-c2ccc3oc4cccc(-c5nc(-c6ccccc6)nc(-c6ccccc6-c6cccc7c6oc6cc8ccccc8cc67)n5)c4c3c2)cc1 ; c1ccc(-c2cc(-c3ccc(-c4cccc5c4oc4cc6ccccc6cc45)cc3)cc(-c3nc(-c4ccccc4)nc(-c4cccc5oc6ccc(-c7ccccc7)cc6c45)n3)c2)cc1 ; c1ccc(-c2nc(-c3cc(-c4cccc5c4oc4cc6ccccc6cc45)c4ccccc4c3)nc(-c3ccc4c(c3)oc3ccccc34)n2)cc1]

The driving voltage, luminous efficiency, and lifetime were measured by applying a current (15 mA/cm²) to the organic light emitting devices manufactured in Examples and Comparative Examples, and the results are shown in Tables 1 to 7 below. Lifetime T95 means the time (hr) required for the luminance to be reduced to 95% of the initial luminance (6,000 nit).

TABLE 1

| | | | Driving | Efficiency | Lifetime | Luminous |
|---|---|---|---|---|---|---|
| Category | First host | Second host | voltage(V) | (cd/A) | T95(hr) | color |
| Ex. 1 | Com. 1 | Com. 2-1 | 3.75 | 25.5 | 262 | Red |
| Ex. 2 | | Com. 2-22 | 3.64 | 24.6 | 240 | Red |
| Ex. 3 | | Com. 2-25 | 3.67 | 24.3 | 274 | Red |
| Ex. 4 | | Com. 2-37 | 3.73 | 24.6 | 232 | Red |
| Ex. 5 | Com. 3 | Com. 2-2 | 3.62 | 25.6 | 273 | Red |
| Ex. 6 | | Com. 2-10 | 3.70 | 25.1 | 236 | Red |
| Ex. 7 | | Com. 2-19 | 3.73 | 24.4 | 217 | Red |
| Ex. 8 | | Com. 2-33 | 3.70 | 24.3 | 201 | Red |
| Ex. 9 | Com. 5 | Com. 2-9 | 3.72 | 25.4 | 263 | Red |
| Ex. 10 | | Com. 2-15 | 3.54 | 25.2 | 272 | Red |
| Ex. 11 | | Com. 2-24 | 3.51 | 24.1 | 230 | Red |
| Ex. 12 | | Com. 2-27 | 3.70 | 23.9 | 224 | Red |
| Ex. 13 | Com. 9 | Com. 2-3 | 3.62 | 24.1 | 211 | Red |
| Ex. 14 | | Com. 2-12 | 3.71 | 26.3 | 239 | Red |
| Ex. 15 | | Com. 2-32 | 3.70 | 23.3 | 217 | Red |
| Ex. 16 | | Com. 2-38 | 3.63 | 26.5 | 231 | Red |
| Ex. 17 | Com. 10 | Com. 2-6 | 3.55 | 26.1 | 243 | Red |
| Ex. 18 | | Com. 2-16 | 3.61 | 25.3 | 257 | Red |
| Ex. 19 | | Com. 2-18 | 3.50 | 24.4 | 222 | Red |
| Ex. 20 | | Com. 2-21 | 3.62 | 24.9 | 241 | Red |
| Ex. 21 | Com. 14 | Com. 2-1 | 3.68 | 25.1 | 233 | Red |
| Ex. 22 | | Com. 2-22 | 3.57 | 24.0 | 215 | Red |
| Ex. 23 | | Com. 2-25 | 3.61 | 25.3 | 241 | Red |
| Ex. 24 | | Com. 2-37 | 3.64 | 24.6 | 207 | Red |
| Ex. 25 | Com. 17 | Com. 2-2 | 3.69 | 25.5 | 258 | Red |
| Ex. 26 | | Com. 2-10 | 3.62 | 25.0 | 231 | Red |
| Ex. 27 | | Com. 2-19 | 3.51 | 24.3 | 224 | Red |
| Ex. 28 | | Com. 2-33 | 3.55 | 24.0 | 230 | Red |
| Ex. 29 | Com. 19 | Com. 2-9 | 3.68 | 25.7 | 271 | Red |
| Ex. 30 | | Com. 2-15 | 3.85 | 26.3 | 286 | Red |
| Ex. 31 | | Com. 2-24 | 3.92 | 24.7 | 240 | Red |
| Ex. 32 | | Com. 2-27 | 3.96 | 24.0 | 233 | Red |
| Ex. 33 | Com. 21 | Com. 2-3 | 3.60 | 24.7 | 221 | Red |
| Ex. 34 | | Com. 2-12 | 3.71 | 26.6 | 254 | Red |
| Ex. 35 | | Com. 2-32 | 3.64 | 24.5 | 217 | Red |
| Ex. 36 | | Com. 2-38 | 3.63 | 26.1 | 238 | Red |

TABLE 2

| | | | Driving | Efficiency | Lifetime | Luminous |
|---|---|---|---|---|---|---|
| Category | First host | Second host | voltage(V) | (cd/A) | T95(hr) | color |
| Ex. 37 | Com. 23 | Com. 2-6 | 3.54 | 26.9 | 251 | Red |
| Ex. 38 | | Com. 2-16 | 3.61 | 25.0 | 248 | Red |
| Ex. 39 | | Com. 2-18 | 3.77 | 24.2 | 213 | Red |
| Ex. 40 | | Com. 2-21 | 3.80 | 24.9 | 204 | Red |
| Ex. 41 | Com. 25 | Com. 2-1 | 3.59 | 26.1 | 252 | Red |
| Ex. 42 | | Com. 2-22 | 3.67 | 24.3 | 213 | Red |
| Ex. 43 | | Com. 2-25 | 3.60 | 25.5 | 257 | Red |
| Ex. 44 | | Com. 2-37 | 3.65 | 24.6 | 220 | Red |
| Ex. 45 | Com. 29 | Com. 2-2 | 3.75 | 26.7 | 255 | Red |
| Ex. 46 | | Com. 2-10 | 3.69 | 26.9 | 229 | Red |
| Ex. 47 | | Com. 2-19 | 3.92 | 24.5 | 237 | Red |
| Ex. 48 | | Com. 2-33 | 3.90 | 24.9 | 223 | Red |
| Ex. 49 | Com. 30 | Com. 2-9 | 3.82 | 26.5 | 261 | Red |
| Ex. 50 | | Com. 2-15 | 3.81 | 26.3 | 264 | Red |
| Ex. 51 | | Com. 2-24 | 3.90 | 24.2 | 231 | Red |
| Ex. 52 | | Com. 2-27 | 4.01 | 24.0 | 208 | Red |
| Ex. 53 | Com. 31 | Com. 2-3 | 3.90 | 24.5 | 221 | Red |
| Ex. 54 | | Com. 2-12 | 3.73 | 26.8 | 246 | Red |
| Ex. 55 | | Com. 2-32 | 3.81 | 24.4 | 214 | Red |
| Ex. 56 | | Com. 2-38 | 3.95 | 26.1 | 239 | Red |
| Ex. 57 | Com. 32 | Com. 2-6 | 3.90 | 26.0 | 256 | Red |
| Ex. 58 | | Com. 2-16 | 3.95 | 26.2 | 242 | Red |
| Ex. 59 | | Com. 2-18 | 3.83 | 24.0 | 224 | Red |
| Ex. 60 | | Com. 2-21 | 3.87 | 24.3 | 202 | Red |
| Ex. 61 | Com. 33 | Com. 2-1 | 3.90 | 26.8 | 251 | Red |
| Ex. 62 | | Com. 2-22 | 3.78 | 24.1 | 230 | Red |
| Ex. 63 | | Com. 2-25 | 3.74 | 25.4 | 264 | Red |
| Ex. 64 | | Com. 2-37 | 3.80 | 24.2 | 217 | Red |
| Ex. 65 | Com. 34 | Com. 2-2 | 3.65 | 25.8 | 253 | Red |
| Ex. 66 | | Com. 2-10 | 3.68 | 25.1 | 231 | Red |
| Ex. 67 | | Com. 2-19 | 3.62 | 24.4 | 219 | Red |
| Ex. 68 | | Com. 2-33 | 3.64 | 24.2 | 218 | Red |
| Ex. 69 | Com. 35 | Com. 2-9 | 3.60 | 25.3 | 275 | Red |
| Ex. 70 | | Com. 2-15 | 3.66 | 25.5 | 270 | Red |
| Ex. 71 | | Com. 2-24 | 3.71 | 24.2 | 222 | Red |
| Ex. 72 | | Com. 2-27 | 3.85 | 24.3 | 213 | Red |

TABLE 3

| | | | Driving | Efficiency | Lifetime | Luminous |
|---|---|---|---|---|---|---|
| Category | First host | Second host | voltage(V) | (cd/A) | T95(hr) | color |
| Ex. 73 | Com. 36 | Com. 2-3 | 3.62 | 24.2 | 225 | Red |
| Ex. 74 | | Com. 2-12 | 3.69 | 26.1 | 274 | Red |
| Ex. 75 | | Com. 2-32 | 3.82 | 24.6 | 227 | Red |
| Ex. 76 | | Com. 2-38 | 3.63 | 26.0 | 269 | Red |
| Ex. 77 | Com. 37 | Com. 2-6 | 3.72 | 25.8 | 275 | Red |
| Ex. 78 | | Com. 2-16 | 3.55 | 26.5 | 261 | Red |
| Ex. 79 | | Com. 2-18 | 3.60 | 24.4 | 238 | Red |
| Ex. 80 | | Com. 2-21 | 3.62 | 24.8 | 220 | Red |
| Ex. 81 | Com. 38 | Com. 2-1 | 3.55 | 26.5 | 277 | Red |
| Ex. 82 | | Com. 2-22 | 3.64 | 24.6 | 236 | Red |
| Ex. 83 | | Com. 2-25 | 3.50 | 26.4 | 265 | Red |
| Ex. 84 | | Com. 2-37 | 3.53 | 24.8 | 240 | Red |
| Ex. 85 | Com. 39 | Com. 2-2 | 3.58 | 26.1 | 284 | Red |
| Ex. 86 | | Com. 2-10 | 3.60 | 26.7 | 230 | Red |
| Ex. 87 | | Com. 2-19 | 3.64 | 24.6 | 242 | Red |
| Ex. 88 | | Com. 2-33 | 3.71 | 24.4 | 221 | Red |
| Ex. 89 | Com. 40 | Com. 2-9 | 3.51 | 25.8 | 279 | Red |
| Ex. 90 | | Com. 2-15 | 3.60 | 26.9 | 287 | Red |
| Ex. 91 | | Com. 2-24 | 3.63 | 24.0 | 219 | Red |
| Ex. 92 | | Com. 2-27 | 3.51 | 24.4 | 230 | Red |
| Ex. 93 | Com. 41 | Com. 2-3 | 3.63 | 24.2 | 227 | Red |
| Ex. 94 | | Com. 2-12 | 3.56 | 26.8 | 267 | Red |
| Ex. 95 | | Com. 2-32 | 3.64 | 24.5 | 213 | Red |
| Ex. 96 | | Com. 2-38 | 3.43 | 26.4 | 258 | Red |
| Ex. 97 | Com. 42 | Com. 2-6 | 3.57 | 26.8 | 267 | Red |
| Ex. 98 | | Com. 2-16 | 3.61 | 25.7 | 270 | Red |
| Ex. 99 | | Com. 2-18 | 3.63 | 24.3 | 233 | Red |
| Ex. 100 | | Com. 2-21 | 3.60 | 24.6 | 238 | Red |

TABLE 4

| | | | Driving | Efficiency | Lifetime | Luminous |
|---|---|---|---|---|---|---|
| Category | First host | Second host | voltage(V) | (cd/A) | T95(hr) | color |
| Comparative | Com. | Com. 2-1 | 4.26 | 19.0 | 180 | Red |
| Ex. 1 | C-1 | | | | | |
| Comparative | | Com. 2-22 | 4.14 | 17.6 | 191 | Red |
| Ex. 2 | | | | | | |
| Comparative | | Com. 2-25 | 4.23 | 18.8 | 172 | Red |
| Ex. 3 | | | | | | |
| Comparative | | Com. 2-37 | 4.20 | 17.7 | 184 | Red |
| Ex. 4 | | | | | | |
| Comparative | Com. | Com. 2-2 | 4.10 | 19.0 | 176 | Red |
| Ex. 5 | C-2 | | | | | |
| Comparative | | Com. 2-10 | 4.23 | 19.7 | 185 | Red |
| Ex. 6 | | | | | | |
| Comparative | | Com. 2-19 | 4.26 | 18.2 | 171 | Red |
| Ex. 7 | | | | | | |
| Comparative | | Com. 2-33 | 4.25 | 17.0 | 168 | Red |
| Ex. 8 | | | | | | |
| Comparative | Com. | Com. 2-9 | 4.23 | 19.2 | 172 | Red |
| Ex. 9 | C-3 | | | | | |
| Comparative | | Com. 2-15 | 4.21 | 18.8 | 173 | Red |
| Ex. 10 | | | | | | |
| Comparative | | Com. 2-24 | 4.32 | 16.4 | 162 | Red |
| Ex. 11 | | | | | | |
| Comparative | | Com. 2-27 | 4.08 | 16.8 | 173 | Red |
| Ex. 12 | | | | | | |
| Comparative | Com. | Com. 2-3 | 4.25 | 17.8 | 162 | Red |
| Ex. 13 | C-4 | | | | | |
| Comparative | | Com. 2-12 | 4.14 | 17.5 | 163 | Red |
| Ex. 14 | | | | | | |
| Comparative | | Com. 2-32 | 4.27 | 16.2 | 164 | Red |
| Ex. 15 | | | | | | |
| Comparative | | Com. 2-38 | 4.30 | 16.5 | 181 | Red |
| Ex. 16 | | | | | | |
| Comparative | Com. | Com. 2-6 | 4.13 | 19.9 | 188 | Red |
| Ex. 17 | C-5 | | | | | |
| Comparative | | Com. 2-16 | 4.18 | 19.8 | 190 | Red |
| Ex. 18 | | | | | | |
| Comparative | | Com. 2-18 | 4.10 | 19.1 | 184 | Red |
| Ex. 19 | | | | | | |
| Comparative | | Com. 2-21 | 4.15 | 19.5 | 187 | Red |
| Ex. 20 | | | | | | |
| Comparative | Com. | Com. 2-1 | 4.23 | 18.6 | 123 | Red |
| Ex. 21 | C-6 | | | | | |
| Comparative | | Com. 2-22 | 4.21 | 17.4 | 112 | Red |
| Ex. 22 | | | | | | |
| Comparative | | Com. 2-25 | 4.25 | 18.3 | 105 | Red |
| Ex. 23 | | | | | | |
| Comparative | | Com. 2-37 | 4.28 | 17.1 | 109 | Red |
| Ex. 24 | | | | | | |
| Comparative | Com. | Com. 2-2 | 4.17 | 17.6 | 72 | Red |
| Ex. 25 | C-7 | | | | | |
| Comparative | | Com. 2-10 | 4.20 | 17.0 | 68 | Red |
| Ex. 26 | | | | | | |
| Comparative | | Com. 2-19 | 4.15 | 16.2 | 63 | Red |
| Ex. 27 | | | | | | |
| Comparative | | Com. 2-33 | 4.14 | 16.3 | 74 | Red |
| Ex. 28 | | | | | | |
| Comparative | Com. | Com. 2-9 | 4.15 | 17.3 | 83 | Red |
| Ex. 29 | C-8 | | | | | |
| Comparative | | Com. 2-15 | 4.11 | 18.4 | 98 | Red |
| Ex. 30 | | | | | | |
| Comparative | | Com. 2-24 | 4.22 | 16.0 | 85 | Red |
| Ex. 31 | | | | | | |
| Comparative | | Com. 2-27 | 4.19 | 15.5 | 81 | Red |
| Ex. 32 | | | | | | |
| Comparative | Com. | Com. 2-3 | 4.23 | 17.5 | 148 | Red |
| Ex. 33 | C-9 | | | | | |
| Comparative | | Com. 2-12 | 4.26 | 18.9 | 154 | Red |
| Ex. 34 | | | | | | |
| Comparative | | Com. 2-32 | 4.28 | 17.8 | 120 | Red |
| Ex. 35 | | | | | | |
| Comparative | | Com. 2-38 | 4.24 | 18.6 | 127 | Red |
| Ex. 36 | | | | | | |

TABLE 5

| | | | Driving | Efficiency | Lifetime | Luminous |
|---|---|---|---|---|---|---|
| Category | First host | Second host | voltage(V) | (cd/A) | T95(hr) | color |
| Comparative | Com. | Com. 2-6 | 4.16 | 19.4 | 168 | Red |
| Ex. 37 | C-10 | | | | | |
| Comparative | | Com. 2-16 | 4.19 | 19.5 | 180 | Red |
| Ex. 38 | | | | | | |
| Comparative | | Com. 2-18 | 4.15 | 18.4 | 174 | Red |
| Ex. 39 | | | | | | |
| Comparative | | Com. 2-21 | 4.12 | 18.1 | 161 | Red |
| Ex. 40 | | | | | | |
| Comparative | Com. | Com. 2-2 | 4.21 | 19.0 | 132 | Red |
| Ex. 41 | C-11 | | | | | |
| Comparative | | Com. 2-10 | 4.28 | 18.3 | 149 | Red |
| Ex. 42 | | | | | | |
| Comparative | | Com. 2-19 | 4.21 | 16.3 | 145 | Red |
| Ex. 43 | | | | | | |
| Comparative | | Com. 2-33 | 4.23 | 16.7 | 142 | Red |
| Ex. 44 | | | | | | |
| Comparative | Com. | Com. 2-9 | 4.24 | 18.5 | 174 | Red |
| Ex. 45 | C-12 | | | | | |
| Comparative | | Com. 2-15 | 4.26 | 18.8 | 174 | Red |
| Ex. 46 | | | | | | |
| Comparative | | Com. 2-24 | 4.28 | 17.2 | 182 | Red |
| Ex. 47 | | | | | | |
| Comparative | | Com. 2-27 | 4.22 | 17.0 | 177 | Red |
| Ex. 48 | | | | | | |

TABLE 6

| | | | Lifetime | Luminous |
|---|---|---|---|---|
| Category | Host | Efficiency (cd/A) | T95(hr) | color |
| Comparative Ex. 49 | Com. 1 | 20.3 | 122 | Red |
| Comparative Ex. 50 | Com. 3 | 21.1 | 135 | Red |
| Comparative Ex. 51 | Com. 5 | 23.2 | 148 | Red |
| Comparative Ex. 52 | Com. 9 | 22.6 | 127 | Red |
| Comparative Ex. 53 | Com. 10 | 21.8 | 143 | Red |
| Comparative Ex. 54 | Com. 14 | 23.2 | 157 | Red |
| Comparative Ex. 55 | Com. 17 | 22.6 | 145 | Red |
| Comparative Ex. 56 | Com. 19 | 21.4 | 128 | Red |
| Comparative Ex. 57 | Com. 21 | 24.5 | 172 | Red |
| Comparative Ex. 58 | Com. 23 | 19.4 | 126 | Red |
| Comparative Ex. 59 | Com. 25 | 20.2 | 129 | Red |
| Comparative Ex. 60 | Com. 29 | 21.3 | 141 | Red |
| Comparative Ex. 61 | Com. 30 | 21.5 | 133 | Red |
| Comparative Ex. 62 | Com. 31 | 20.2 | 145 | Red |
| Comparative Ex. 63 | Com. 32 | 21.6 | 157 | Red |
| Comparative Ex. 64 | Com. 33 | 22.3 | 140 | Red |
| Comparative Ex. 65 | Com. 34 | 21.6 | 152 | Red |
| Comparative Ex. 66 | Com. 35 | 22.2 | 143 | Red |
| Comparative Ex. 67 | Com. 36 | 22.8 | 142 | Red |
| Comparative Ex. 68 | Com. 37 | 21.6 | 158 | Red |
| Comparative Ex. 69 | Com. 38 | 22.3 | 141 | Red |
| Comparative Ex. 70 | Com. 39 | 21.5 | 151 | Red |
| Comparative Ex. 71 | Com. 40 | 20.7 | 160 | Red |
| Comparative Ex. 72 | Com. 41 | 22.6 | 159 | Red |
| Comparative Ex. 73 | Com. 42 | 23.8 | 163 | Red |

TABLE 7

| | | | Lifetime | Luminous |
|---|---|---|---|---|
| Category | Host | Efficiency (cd/A) | T95(hr) | color |
| Comparative Ex. 74 | Com. C-1 | 17.4 | 107 | Red |
| Comparative Ex. 75 | Com. C-2 | 16.1 | 83 | Red |
| Comparative Ex. 76 | Com. C-3 | 16.4 | 94 | Red |
| Comparative Ex. 77 | Com. C-4 | 16.0 | 87 | Red |
| Comparative Ex. 78 | Com. C-5 | 18.7 | 110 | Red |
| Comparative Ex. 79 | Com. C-6 | 16.5 | 47 | Red |
| Comparative Ex. 80 | Com. C-7 | 15.3 | 22 | Red |
| Comparative Ex. 81 | Com. C-8 | 15.1 | 37 | Red |
| Comparative Ex. 82 | Com. C-9 | 17.3 | 75 | Red |
| Comparative Ex. 83 | Com. C-10 | 17.5 | 92 | Red |
| Comparative Ex. 84 | Com. C-11 | 15.8 | 63 | Red |
| Comparative Ex. 85 | Com. C-12 | 16.1 | 78 | Red |

As shown in Tables above, the organic light emitting devices of Examples, in which the first compound represented by Chemical Formula 1 and the second compound represented by Chemical Formula 2 were simultaneously used as the host materials of the light emitting layer, exhibited excellent luminous efficiency and remarkably improved lifetime characteristics as compared with the organic light emitting devices of Comparative Examples in which only one of the compounds represented by Chemical Formulas 1 and 2 was used (Table 6), or both of them were not used (Table 7). Specifically, the devices according to Examples exhibited higher efficiency and longer lifetime than the devices of Comparative Examples in which the compound represented by Chemical Formula 1 was used as a single host. In addition, the devices according to Examples exhibited improved efficiency and lifetime characteristics even as compared with the devices of Comparative Examples in which Compounds C-1 to C-12 of Comparative Examples were employed as the first host, and the compound represented by Chemical Formula 2 as the second host. Thereby, it was confirmed that when combination of the first compound represented by Chemical Formula 1 and the second compound represented by Chemical Formula 2 was used as a co-host, energy transfer to the red dopant was effectively performed in the red light emitting layer. This can be judged to be because the first compound has high electron and hole stability, and further, because the amount of holes increased along with the simultaneous use of the second compound and thus, the electrons and holes in the red light emitting layer maintained a more stable balance.

Therefore, when the first compound and the second compound are simultaneously used as the host materials of the organic light emitting devices, it was confirmed that the driving voltage, luminous efficiency and/or lifetime characteristics of the organic light emitting devices can be improved. In general, considering that the luminous efficiency and lifetime characteristics of an organic light emitting devices have a trade-off relationship with each other, this can be considered that the organic light emitting devices adopting combination of the compounds of the present disclosure exhibit remarkably improved device characteristics as compared with the devices of Comparative Examples.

### DESCRIPTION OF REFERENCE NUMERALS

| | |
|---|---|
| 1: substrate | 2: anode |
| 3: light emitting layer | 4: cathode |
| 5: hole transport layer | 6: electron transport layer |

1. An organic light emitting device comprising:
an anode,
a cathode, and
a light emitting layer interposed between the anode and the cathode,
wherein the light emitting layer comprises a compound represented by the following Chemical Formula 1 and a compound represented by the following Chemical Formula 2, 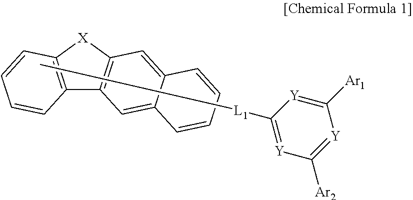
wherein in Chemical Formula 1,
X is O or S,
each Y is independently N or CH, with the proviso that at least one Y is N,
L₁ is a single bond; or a substituted or unsubstituted C₆₋₆₀ arylene,
Ar₁ and Ar₂ are each independently a substituted or unsubstituted C₆₋₆₀ aryl; or a substituted or unsubstituted C₂₋₆₀ heteroaryl containing any one or more selected from the group consisting of N, O and S, 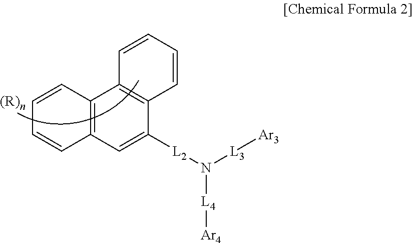
wherein in Chemical Formula 2,
L₂ is a substituted or unsubstituted C₆₋₆₀ arylene,
L₃ and L₄ are each independently a single bond; or a substituted or unsubstituted C₆₋₆₀ arylene,
Ar₃ and Ar₄ are each independently a substituted or unsubstituted C₆₋₆₀ aryl; or a substituted or unsubstituted C₂₋₆₀ heteroaryl containing any one or more selected from the group consisting of N, O and S,
R is deuterium; or a substituted or unsubstituted C₆₋₆₀ aryl, and
n is an integer of 0 to 9.

2. The organic light emitting device according to claim 1,
wherein each Y is N.

3. The organic light emitting device according to claim 1,
wherein L₁ is a single bond; phenylene; or naphthylene.

4. The organic light emitting device according to claim 1,
wherein L₁ is a single bond;

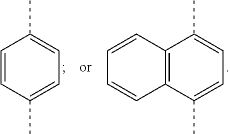

5. The organic light emitting device according to claim 1,
wherein Ar₁ and Ar₂ are each independently phenyl, biphenyl, terphenyl, naphthyl, phenanthrenyl, (phenyl)naphthyl, (naphthyl)phenyl, dimethylfluorenyl, diphenylfluorenyl, dibenzofuranyl, dibenzothiophenyl, carbazole-9-yl, 9-phenyl-9H-carbazolyl,
each of which is independently unsubstituted or substituted with at least one deuterium.

6. The organic light emitting device according to claim 1,
wherein Ar₁ is phenyl, biphenyl, or naphthyl,
each of which is unsubstituted or substituted with at least one deuterium, and
Ar₂ is phenyl, biphenyl, terphenyl, naphthyl, phenanthrenyl, (phenyl)naphthyl, (naphthyl)phenyl, dimethylfluorenyl, diphenylfluorenyl, dibenzofuranyl, dibenzothiophenyl, carbazol-9-yl, 9-phenyl-9H-carbazolyl,
each of which is unsubstituted or substituted with at least one deuterium.

7. The organic light emitting device according to claim 1,
wherein the compound represented by Chemical Formula 1 is any one selected from the group consisting of the following:

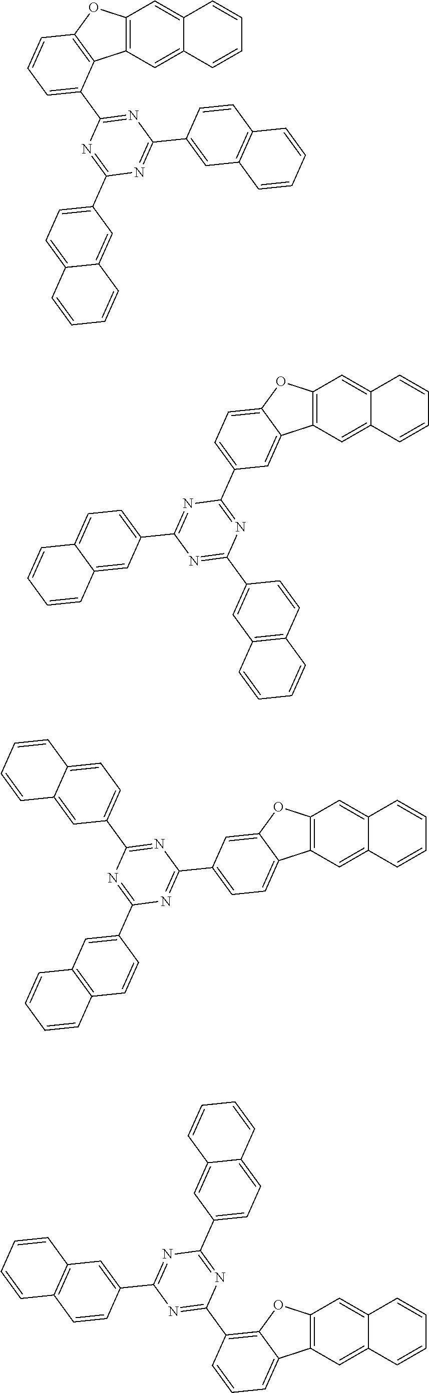 [SMILES: c1ccc2cc(-c3nc(-c4ccc5ccccc5c4)nc(-c4cccc5oc6cc7ccccc7cc6c45)n3)ccc2c1 ; c1ccc2cc(-c3nc(-c4ccc5ccccc5c4)nc(-c4ccc5oc6cc7ccccc7cc6c5c4)n3)ccc2c1 ; c1ccc2cc(-c3nc(-c4ccc5ccccc5c4)nc(-c4ccc5c(c4)oc4cc6ccccc6cc45)n3)ccc2c1 ; c1ccc2cc(-c3nc(-c4ccc5ccccc5c4)nc(-c4cccc5c4oc4cc6ccccc6cc45)n3)ccc2c1]

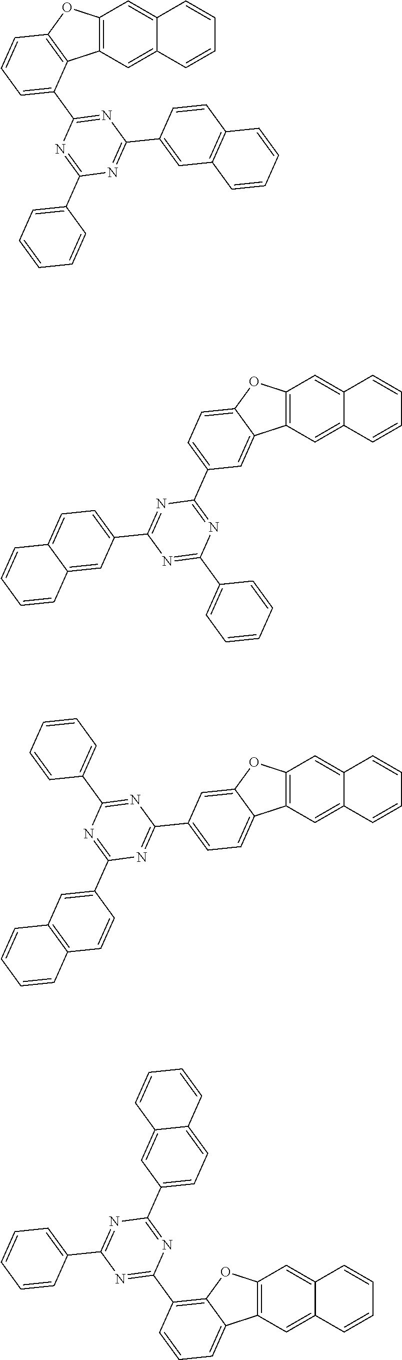 [SMILES: c1ccc(-c2nc(-c3ccc4ccccc4c3)nc(-c3cccc4oc5cc6ccccc6cc5c34)n2)cc1 ; c1ccc(-c2nc(-c3ccc4ccccc4c3)nc(-c3ccc4oc5cc6ccccc6cc5c4c3)n2)cc1 ; c1ccc(-c2nc(-c3ccc4ccccc4c3)nc(-c3ccc4c(c3)oc3cc5ccccc5cc34)n2)cc1 ; c1ccc(-c2nc(-c3ccc4ccccc4c3)nc(-c3cccc4c3oc3cc5ccccc5cc34)n2)cc1]

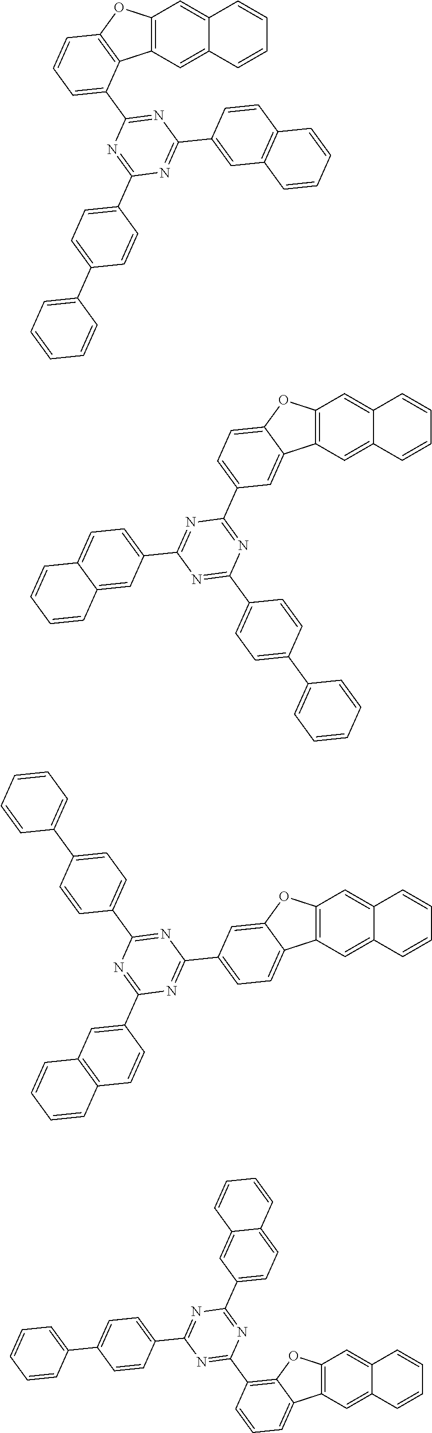 [SMILES: c1ccc(-c2ccc(-c3nc(-c4ccc5ccccc5c4)nc(-c4cccc5oc6cc7ccccc7cc6c45)n3)cc2)cc1 ; c1ccc(-c2ccc(-c3nc(-c4ccc5ccccc5c4)nc(-c4ccc5oc6cc7ccccc7cc6c5c4)n3)cc2)cc1 ; c1ccc(-c2ccc(-c3nc(-c4ccc5ccccc5c4)nc(-c4ccc5c(c4)oc4cc6ccccc6cc45)n3)cc2)cc1 ; c1ccc(-c2ccc(-c3nc(-c4ccc5ccccc5c4)nc(-c4cccc5c4oc4cc6ccccc6cc45)n3)cc2)cc1]

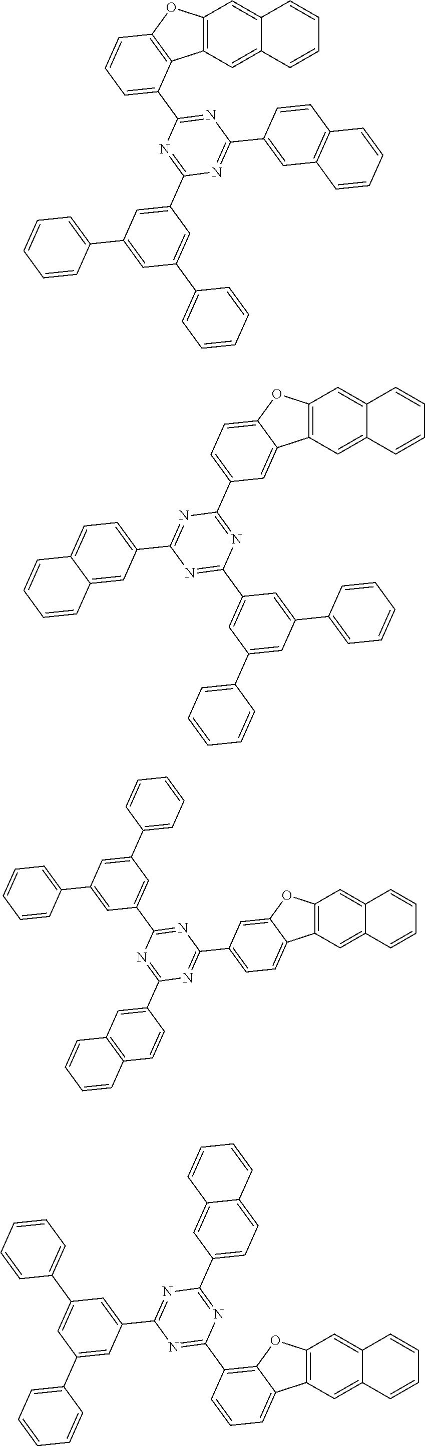 [SMILES: c1ccc(-c2cc(-c3ccccc3)cc(-c3nc(-c4ccc5ccccc5c4)nc(-c4cccc5oc6cc7ccccc7cc6c45)n3)c2)cc1 ; c1ccc(-c2cc(-c3ccccc3)cc(-c3nc(-c4ccc5ccccc5c4)nc(-c4ccc5oc6cc7ccccc7cc6c5c4)n3)c2)cc1 ; c1ccc(-c2cc(-c3ccccc3)cc(-c3nc(-c4ccc5ccccc5c4)nc(-c4ccc5c(c4)oc4cc6ccccc6cc45)n3)c2)cc1 ; c1ccc(-c2cc(-c3ccccc3)cc(-c3nc(-c4ccc5ccccc5c4)nc(-c4cccc5c4oc4cc6ccccc6cc45)n3)c2)cc1]

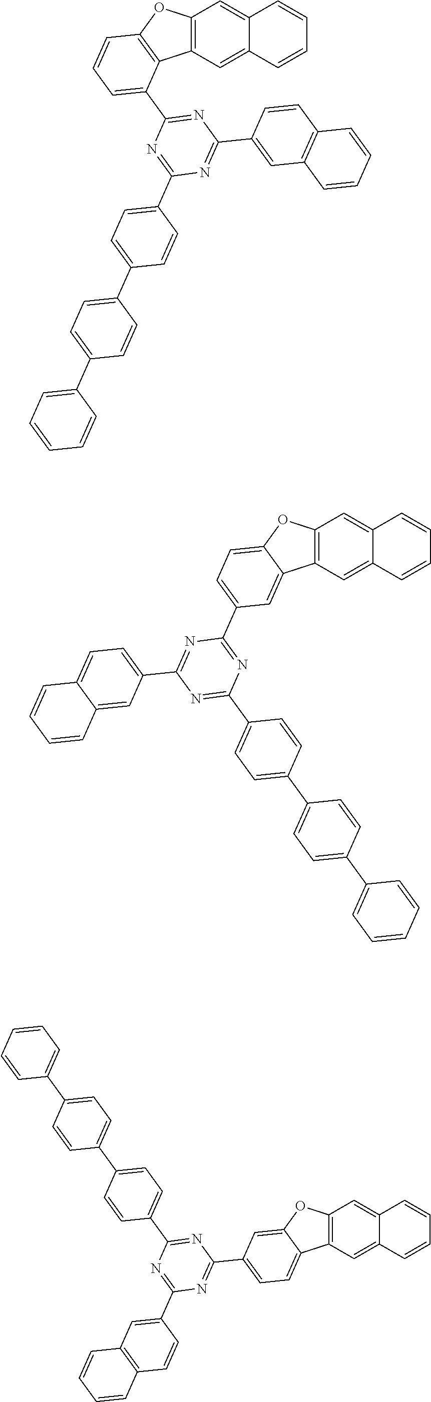 [SMILES: c1ccc(-c2ccc(-c3ccc(-c4nc(-c5ccc6ccccc6c5)nc(-c5cccc6oc7cc8ccccc8cc7c56)n4)cc3)cc2)cc1 ; c1ccc(-c2ccc(-c3ccc(-c4nc(-c5ccc6ccccc6c5)nc(-c5ccc6oc7cc8ccccc8cc7c6c5)n4)cc3)cc2)cc1 ; c1ccc(-c2ccc(-c3ccc(-c4nc(-c5ccc6ccccc6c5)nc(-c5ccc6c(c5)oc5cc7ccccc7cc56)n4)cc3)cc2)cc1]

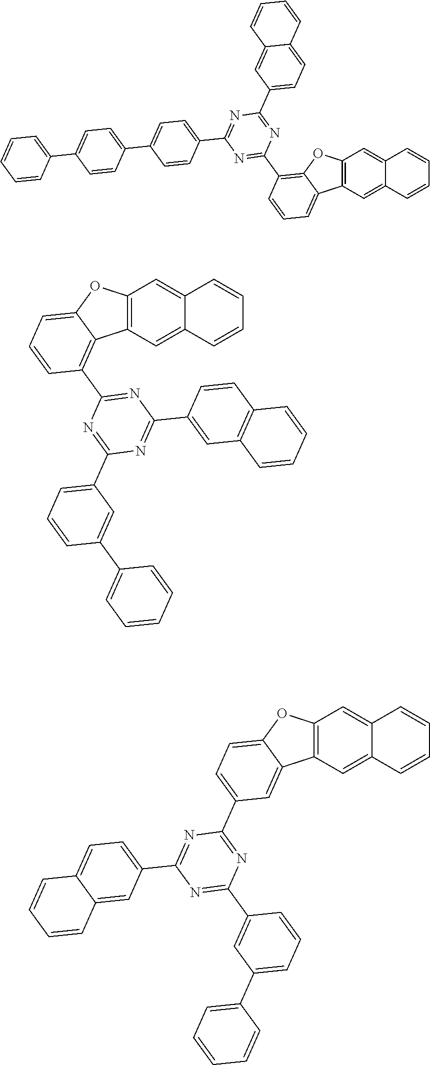 [SMILES: c1ccc(-c2ccc(-c3ccc(-c4nc(-c5ccc6ccccc6c5)nc(-c5cccc6c5oc5cc7ccccc7cc56)n4)cc3)cc2)cc1 ; c1ccc(-c2cccc(-c3nc(-c4ccc5ccccc5c4)nc(-c4cccc5oc6cc7ccccc7cc6c45)n3)c2)cc1 ; c1ccc(-c2cccc(-c3nc(-c4ccc5ccccc5c4)nc(-c4ccc5oc6cc7ccccc7cc6c5c4)n3)c2)cc1]

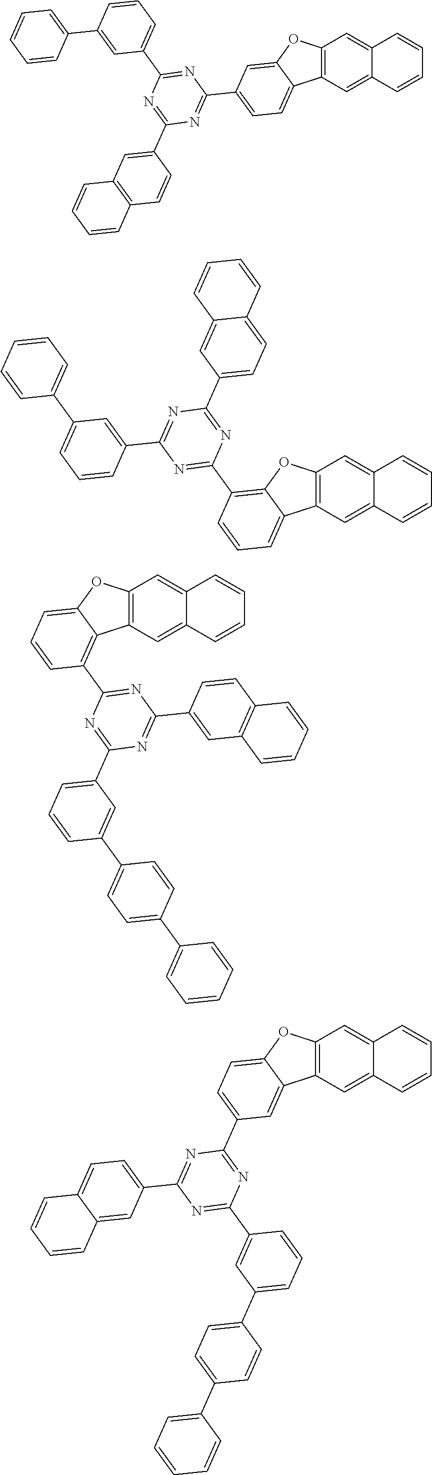

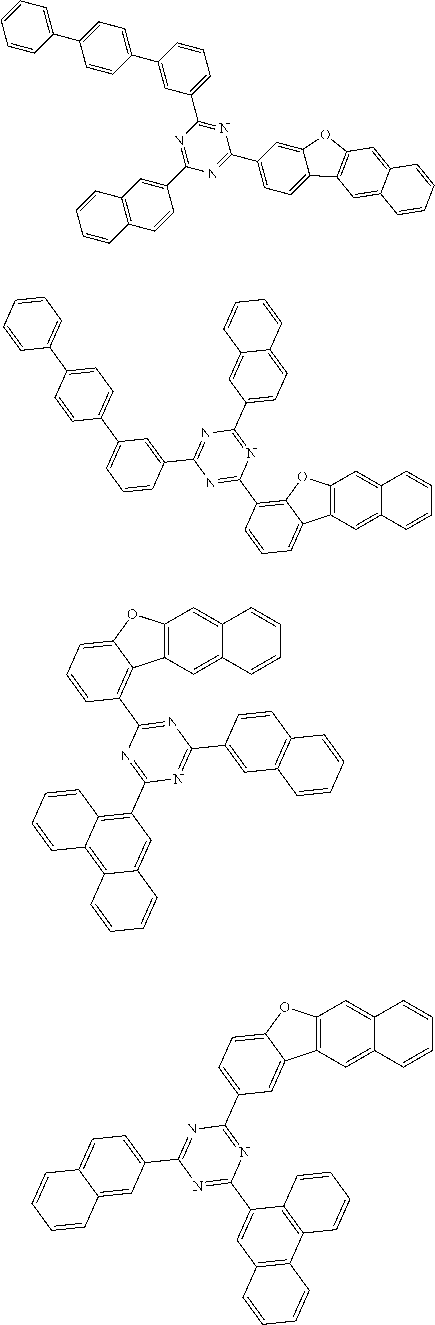 [SMILES: c1ccc(-c2ccc(-c3cccc(-c4nc(-c5ccc6ccccc6c5)nc(-c5ccc6c(c5)oc5cc7ccccc7cc56)n4)c3)cc2)cc1 ; c1ccc(-c2ccc(-c3cccc(-c4nc(-c5ccc6ccccc6c5)nc(-c5cccc6c5oc5cc7ccccc7cc56)n4)c3)cc2)cc1 ; c1ccc2cc(-c3nc(-c4cc5ccccc5c5ccccc45)nc(-c4cccc5oc6cc7ccccc7cc6c45)n3)ccc2c1 ; c1ccc2cc(-c3nc(-c4ccc5oc6cc7ccccc7cc6c5c4)nc(-c4cc5ccccc5c5ccccc45)n3)ccc2c1]

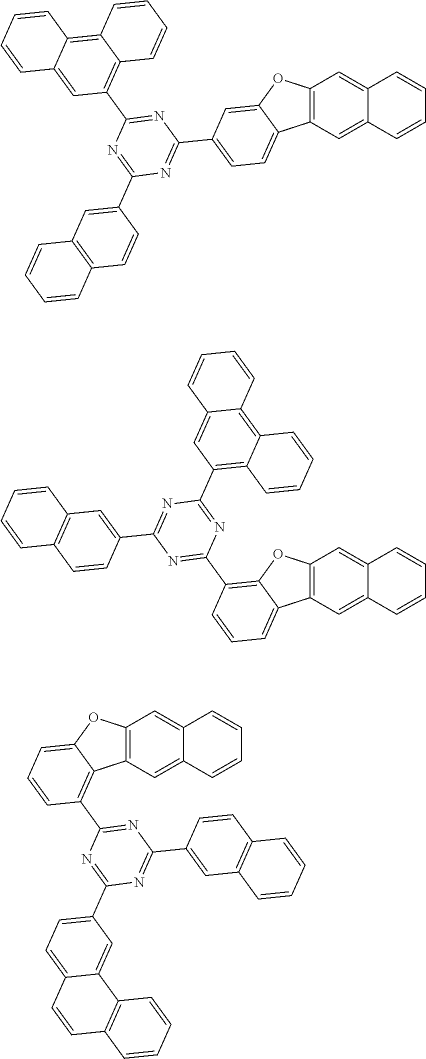 [SMILES: c1ccc2cc(-c3nc(-c4ccc5c(c4)oc4cc6ccccc6cc45)nc(-c4cc5ccccc5c5ccccc45)n3)ccc2c1 ; c1ccc2cc(-c3nc(-c4cc5ccccc5c5ccccc45)nc(-c4cccc5c4oc4cc6ccccc6cc45)n3)ccc2c1 ; c1ccc2cc(-c3nc(-c4ccc5ccc6ccccc6c5c4)nc(-c4cccc5oc6cc7ccccc7cc6c45)n3)ccc2c1]

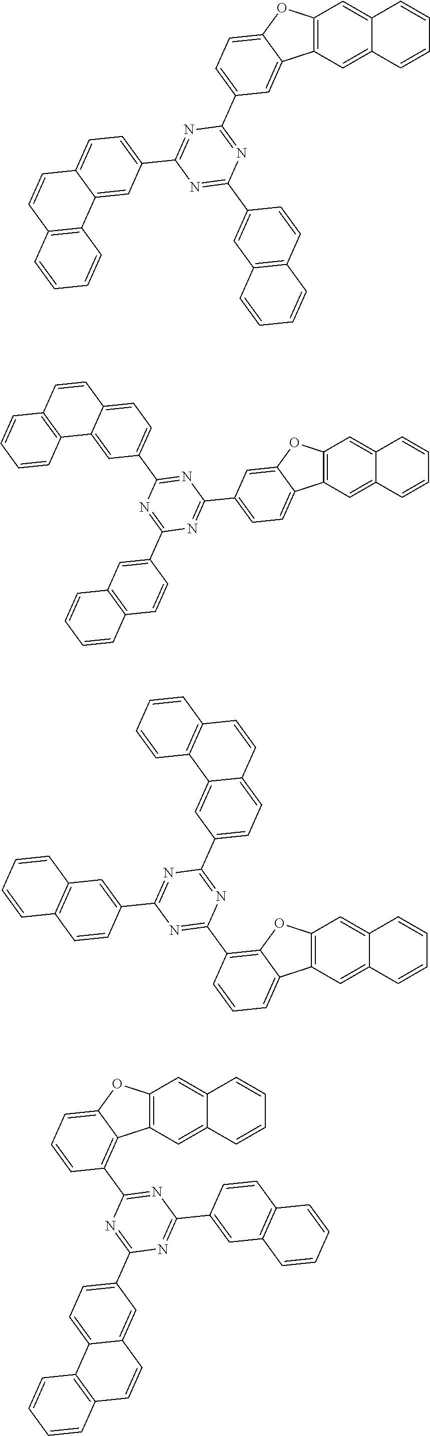 [SMILES: c1ccc2cc(-c3nc(-c4ccc5ccc6ccccc6c5c4)nc(-c4ccc5oc6cc7ccccc7cc6c5c4)n3)ccc2c1 ; c1ccc2cc(-c3nc(-c4ccc5c(c4)oc4cc6ccccc6cc45)nc(-c4ccc5ccc6ccccc6c5c4)n3)ccc2c1 ; c1ccc2cc(-c3nc(-c4ccc5ccc6ccccc6c5c4)nc(-c4cccc5c4oc4cc6ccccc6cc45)n3)ccc2c1 ; c1ccc2cc(-c3nc(-c4ccc5c(ccc6ccccc65)c4)nc(-c4cccc5oc6cc7ccccc7cc6c45)n3)ccc2c1]

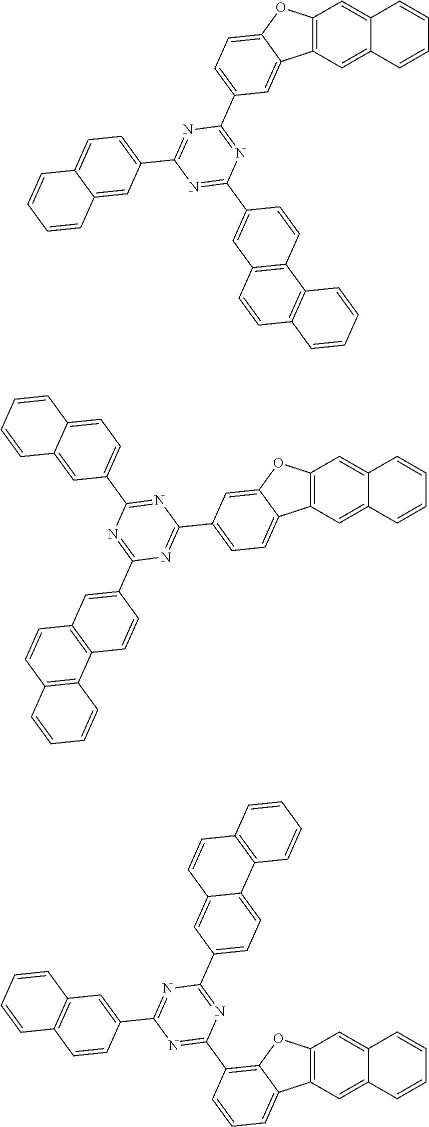 [SMILES: c1ccc2cc(-c3nc(-c4ccc5c(ccc6ccccc65)c4)nc(-c4ccc5oc6cc7ccccc7cc6c5c4)n3)ccc2c1 ; c1ccc2cc(-c3nc(-c4ccc5c(ccc6ccccc65)c4)nc(-c4ccc5c(c4)oc4cc6ccccc6cc45)n3)ccc2c1 ; c1ccc2cc(-c3nc(-c4ccc5c(ccc6ccccc65)c4)nc(-c4cccc5c4oc4cc6ccccc6cc45)n3)ccc2c1]

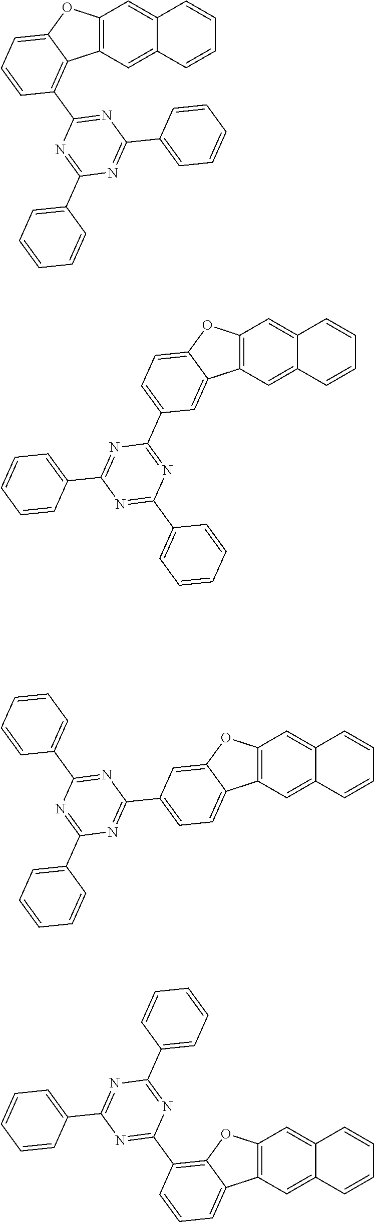 [SMILES: c1ccc(-c2nc(-c3ccccc3)nc(-c3cccc4oc5cc6ccccc6cc5c34)n2)cc1 ; c1ccc(-c2nc(-c3ccccc3)nc(-c3ccc4oc5cc6ccccc6cc5c4c3)n2)cc1 ; c1ccc(-c2nc(-c3ccccc3)nc(-c3ccc4c(c3)oc3cc5ccccc5cc34)n2)cc1 ; c1ccc(-c2nc(-c3ccccc3)nc(-c3cccc4c3oc3cc5ccccc5cc34)n2)cc1]

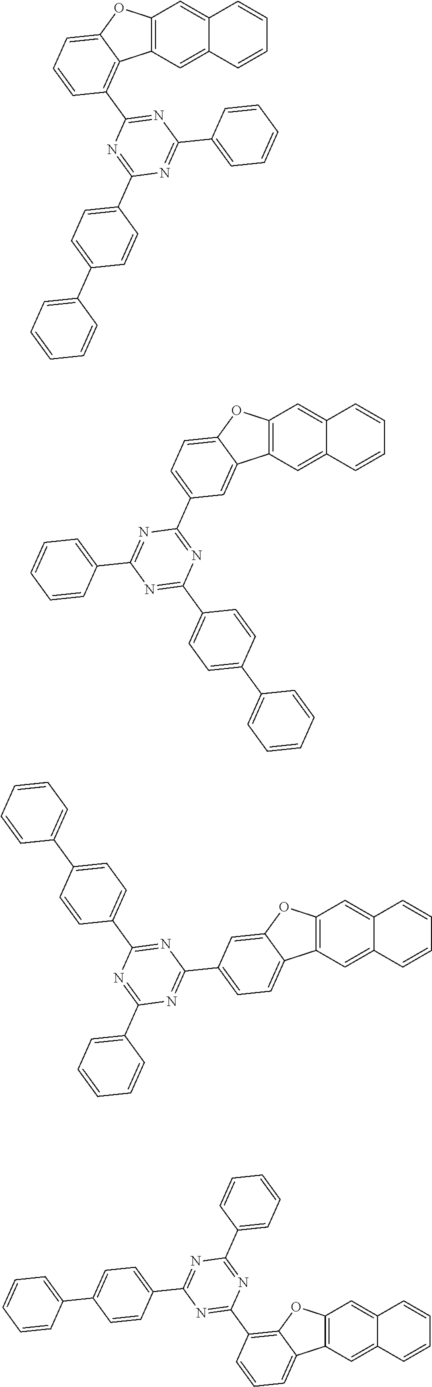 [SMILES: c1ccc(-c2ccc(-c3nc(-c4ccccc4)nc(-c4cccc5oc6cc7ccccc7cc6c45)n3)cc2)cc1 ; c1ccc(-c2ccc(-c3nc(-c4ccccc4)nc(-c4ccc5oc6cc7ccccc7cc6c5c4)n3)cc2)cc1 ; c1ccc(-c2ccc(-c3nc(-c4ccccc4)nc(-c4ccc5c(c4)oc4cc6ccccc6cc45)n3)cc2)cc1 ; c1ccc(-c2ccc(-c3nc(-c4ccccc4)nc(-c4cccc5c4oc4cc6ccccc6cc45)n3)cc2)cc1]

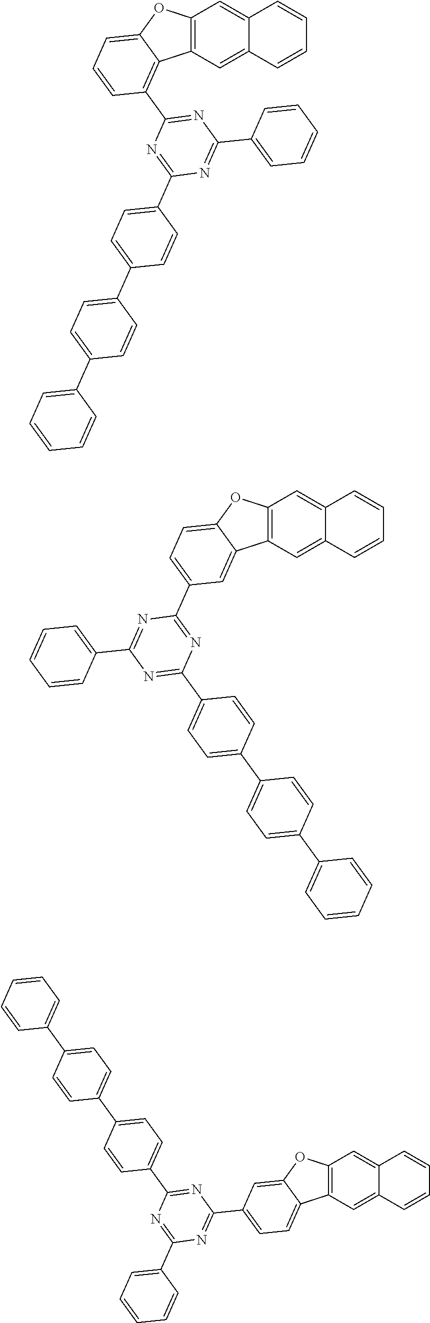 [SMILES: c1ccc(-c2ccc(-c3ccc(-c4nc(-c5ccccc5)nc(-c5cccc6oc7cc8ccccc8cc7c56)n4)cc3)cc2)cc1 ; c1ccc(-c2ccc(-c3ccc(-c4nc(-c5ccccc5)nc(-c5ccc6oc7cc8ccccc8cc7c6c5)n4)cc3)cc2)cc1 ; c1ccc(-c2ccc(-c3ccc(-c4nc(-c5ccccc5)nc(-c5ccc6c(c5)oc5cc7ccccc7cc56)n4)cc3)cc2)cc1]

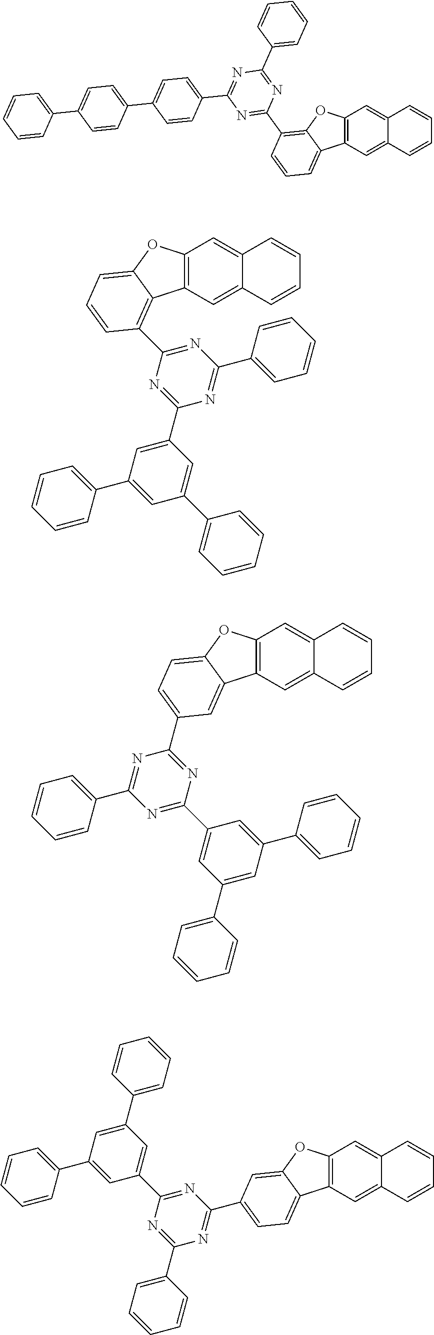 [SMILES: c1ccc(-c2ccc(-c3ccc(-c4nc(-c5ccccc5)nc(-c5cccc6c5oc5cc7ccccc7cc56)n4)cc3)cc2)cc1 ; c1ccc(-c2cc(-c3ccccc3)cc(-c3nc(-c4ccccc4)nc(-c4cccc5oc6cc7ccccc7cc6c45)n3)c2)cc1 ; c1ccc(-c2cc(-c3ccccc3)cc(-c3nc(-c4ccccc4)nc(-c4ccc5oc6cc7ccccc7cc6c5c4)n3)c2)cc1 ; c1ccc(-c2cc(-c3ccccc3)cc(-c3nc(-c4ccccc4)nc(-c4ccc5c(c4)oc4cc6ccccc6cc45)n3)c2)cc1]

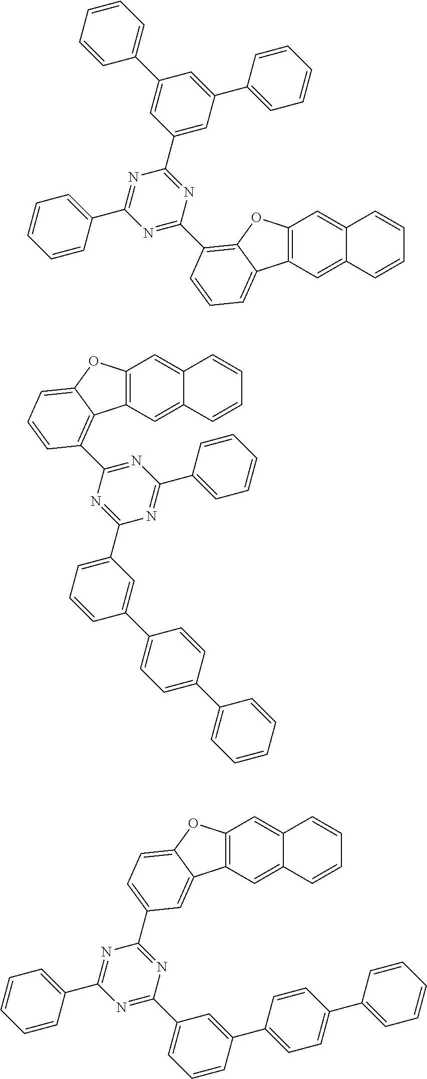 [SMILES: c1ccc(-c2cc(-c3ccccc3)cc(-c3nc(-c4ccccc4)nc(-c4cccc5c4oc4cc6ccccc6cc45)n3)c2)cc1 ; c1ccc(-c2ccc(-c3cccc(-c4nc(-c5ccccc5)nc(-c5cccc6oc7cc8ccccc8cc7c56)n4)c3)cc2)cc1 ; c1ccc(-c2ccc(-c3cccc(-c4nc(-c5ccccc5)nc(-c5ccc6oc7cc8ccccc8cc7c6c5)n4)c3)cc2)cc1]

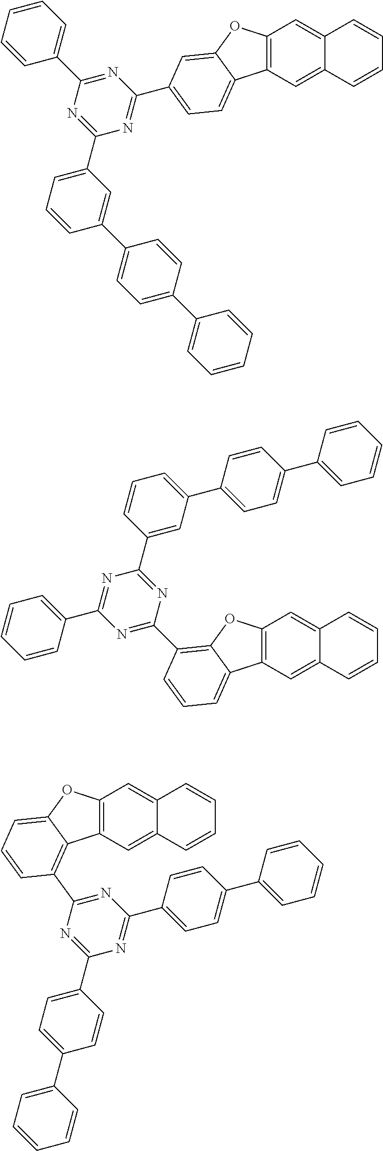 [SMILES: c1ccc(-c2ccc(-c3cccc(-c4nc(-c5ccccc5)nc(-c5ccc6c(c5)oc5cc7ccccc7cc56)n4)c3)cc2)cc1 ; c1ccc(-c2ccc(-c3cccc(-c4nc(-c5ccccc5)nc(-c5cccc6c5oc5cc7ccccc7cc56)n4)c3)cc2)cc1 ; c1ccc(-c2ccc(-c3nc(-c4ccc(-c5ccccc5)cc4)nc(-c4cccc5oc6cc7ccccc7cc6c45)n3)cc2)cc1]

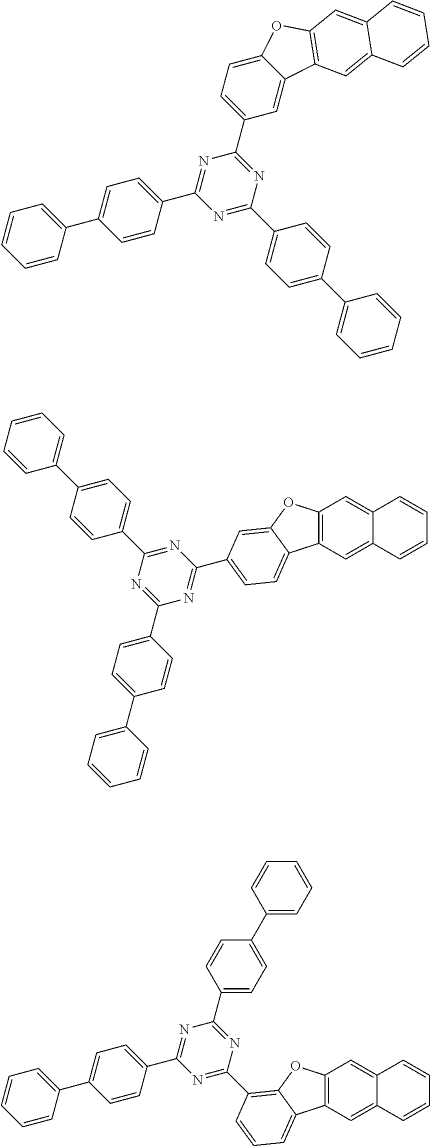 [SMILES: c1ccc(-c2ccc(-c3nc(-c4ccc(-c5ccccc5)cc4)nc(-c4ccc5oc6cc7ccccc7cc6c5c4)n3)cc2)cc1 ; c1ccc(-c2ccc(-c3nc(-c4ccc(-c5ccccc5)cc4)nc(-c4ccc5c(c4)oc4cc6ccccc6cc45)n3)cc2)cc1 ; c1ccc(-c2ccc(-c3nc(-c4ccc(-c5ccccc5)cc4)nc(-c4cccc5c4oc4cc6ccccc6cc45)n3)cc2)cc1]

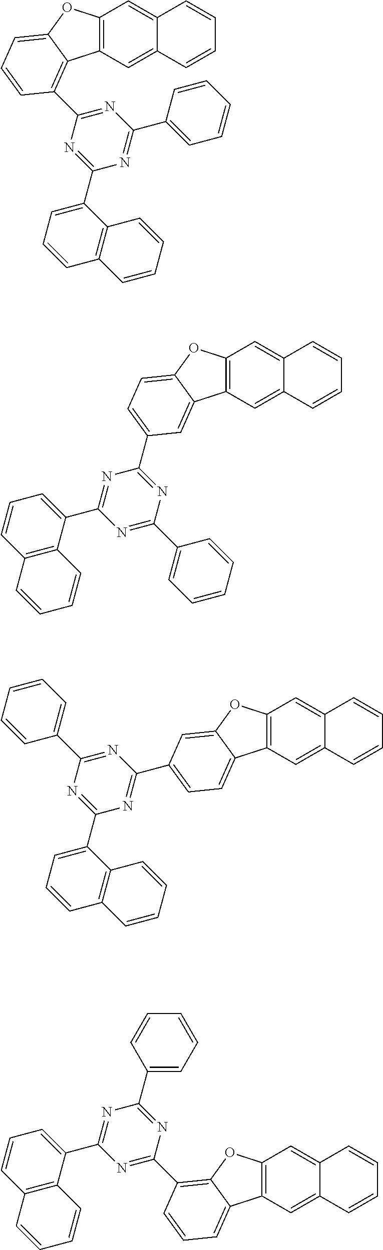 [SMILES: c1ccc(-c2nc(-c3cccc4ccccc34)nc(-c3cccc4oc5cc6ccccc6cc5c34)n2)cc1 ; c1ccc(-c2nc(-c3ccc4oc5cc6ccccc6cc5c4c3)nc(-c3cccc4ccccc34)n2)cc1 ; c1ccc(-c2nc(-c3ccc4c(c3)oc3cc5ccccc5cc34)nc(-c3cccc4ccccc34)n2)cc1 ; c1ccc(-c2nc(-c3cccc4ccccc34)nc(-c3cccc4c3oc3cc5ccccc5cc34)n2)cc1]

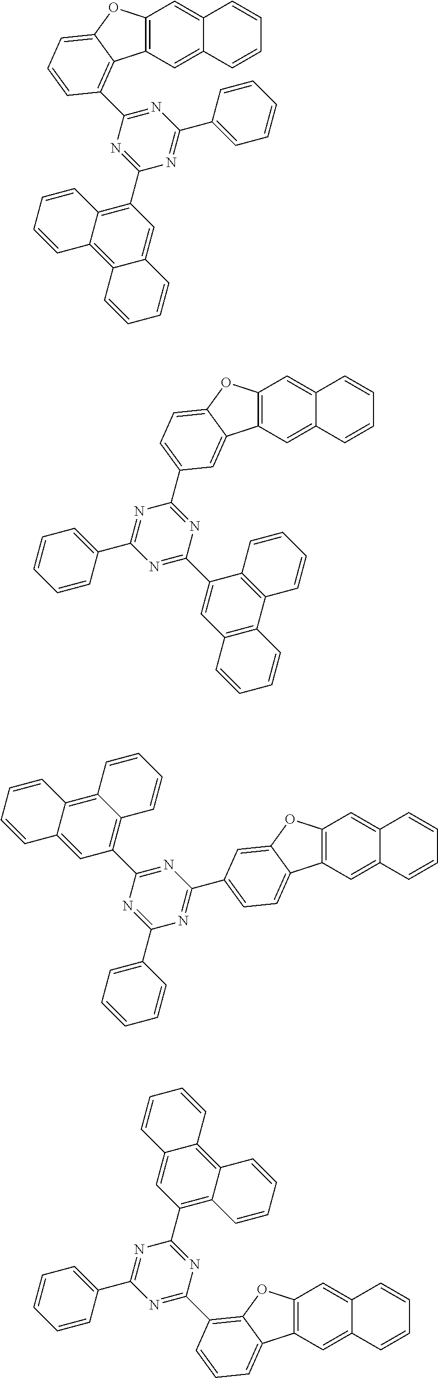 [SMILES: c1ccc(-c2nc(-c3cc4ccccc4c4ccccc34)nc(-c3cccc4oc5cc6ccccc6cc5c34)n2)cc1 ; c1ccc(-c2nc(-c3ccc4oc5cc6ccccc6cc5c4c3)nc(-c3cc4ccccc4c4ccccc34)n2)cc1 ; c1ccc(-c2nc(-c3ccc4c(c3)oc3cc5ccccc5cc34)nc(-c3cc4ccccc4c4ccccc34)n2)cc1 ; c1ccc(-c2nc(-c3cc4ccccc4c4ccccc34)nc(-c3cccc4c3oc3cc5ccccc5cc34)n2)cc1]

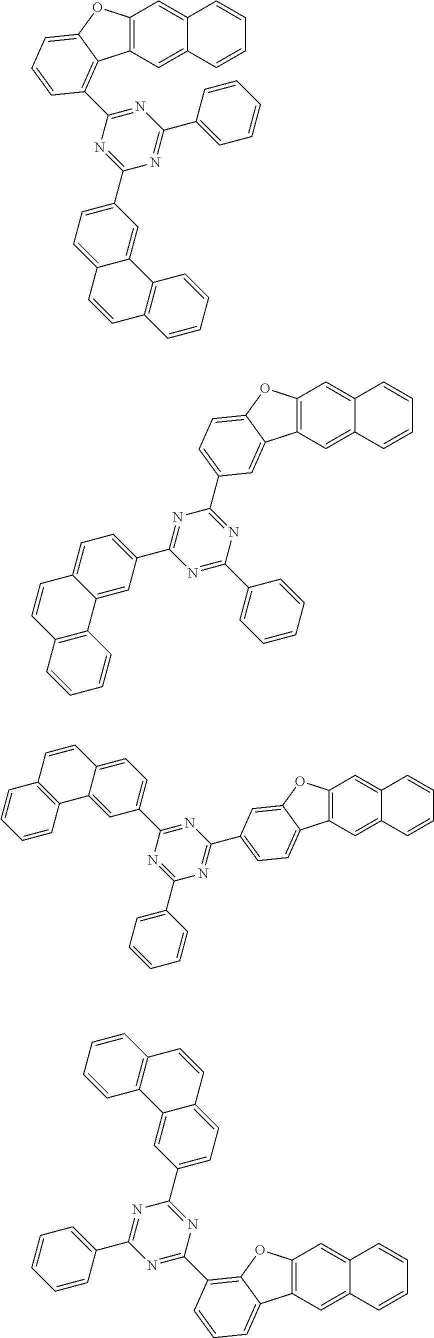 [SMILES: c1ccc(-c2nc(-c3ccc4ccc5ccccc5c4c3)nc(-c3cccc4oc5cc6ccccc6cc5c34)n2)cc1 ; c1ccc(-c2nc(-c3ccc4ccc5ccccc5c4c3)nc(-c3ccc4oc5cc6ccccc6cc5c4c3)n2)cc1 ; c1ccc(-c2nc(-c3ccc4c(c3)oc3cc5ccccc5cc34)nc(-c3ccc4ccc5ccccc5c4c3)n2)cc1 ; c1ccc(-c2nc(-c3ccc4ccc5ccccc5c4c3)nc(-c3cccc4c3oc3cc5ccccc5cc34)n2)cc1]

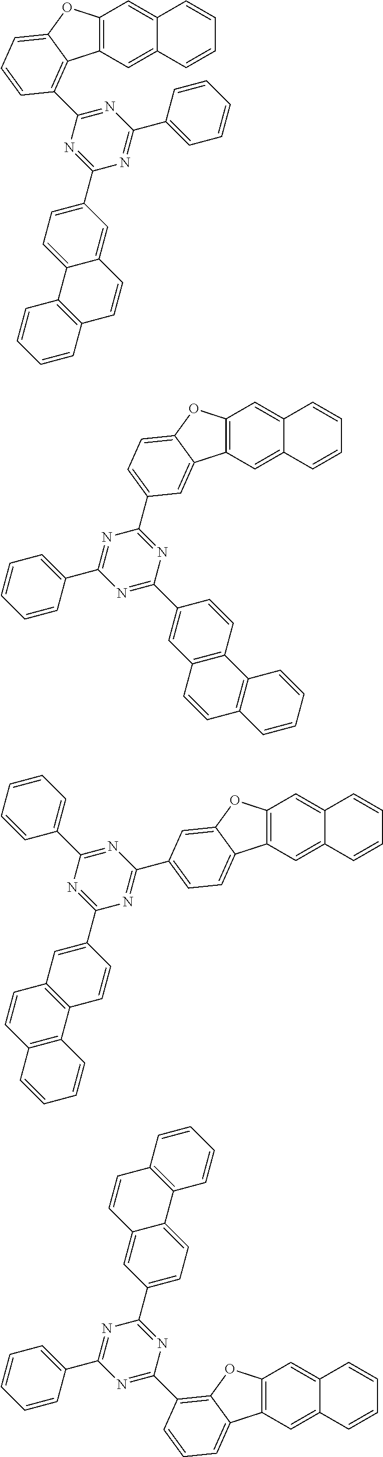 [SMILES: c1ccc(-c2nc(-c3ccc4c(ccc5ccccc54)c3)nc(-c3cccc4oc5cc6ccccc6cc5c34)n2)cc1 ; c1ccc(-c2nc(-c3ccc4c(ccc5ccccc54)c3)nc(-c3ccc4oc5cc6ccccc6cc5c4c3)n2)cc1 ; c1ccc(-c2nc(-c3ccc4c(ccc5ccccc54)c3)nc(-c3ccc4c(c3)oc3cc5ccccc5cc34)n2)cc1 ; c1ccc(-c2nc(-c3ccc4c(ccc5ccccc54)c3)nc(-c3cccc4c3oc3cc5ccccc5cc34)n2)cc1]

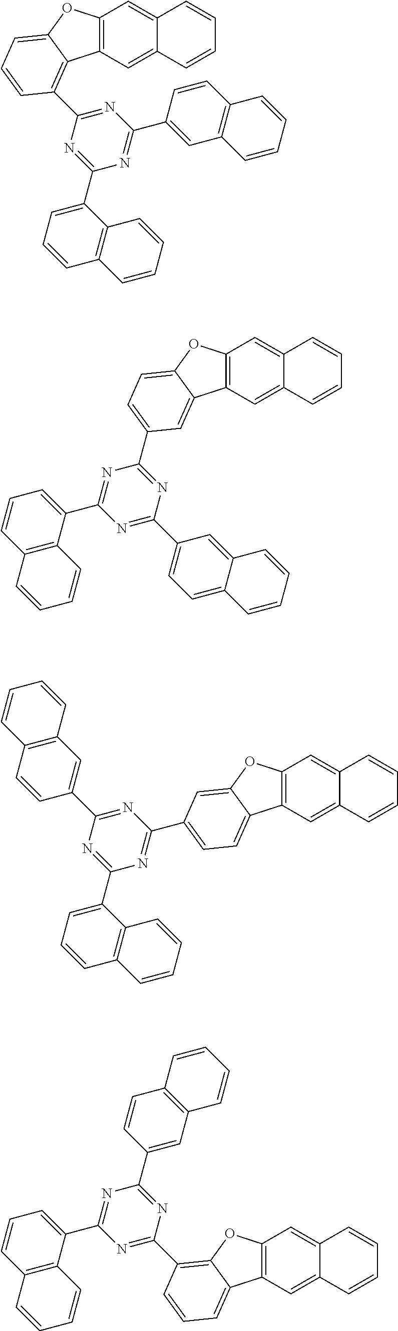 [SMILES: c1ccc2cc(-c3nc(-c4cccc5ccccc45)nc(-c4cccc5oc6cc7ccccc7cc6c45)n3)ccc2c1 ; c1ccc2cc(-c3nc(-c4ccc5oc6cc7ccccc7cc6c5c4)nc(-c4cccc5ccccc45)n3)ccc2c1 ; c1ccc2cc(-c3nc(-c4ccc5c(c4)oc4cc6ccccc6cc45)nc(-c4cccc5ccccc45)n3)ccc2c1 ; c1ccc2cc(-c3nc(-c4cccc5ccccc45)nc(-c4cccc5c4oc4cc6ccccc6cc45)n3)ccc2c1]

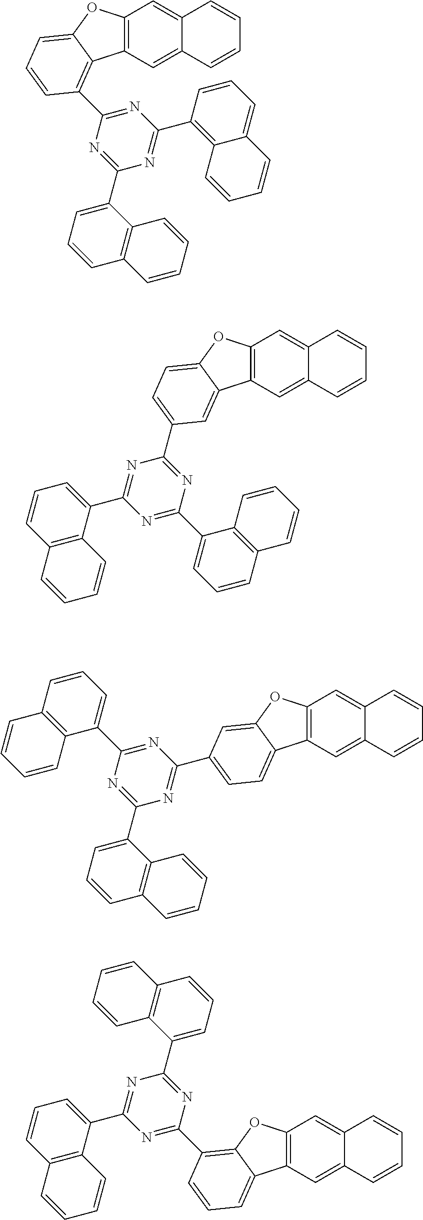 [SMILES: c1ccc2cc3c(cc2c1)oc1cccc(-c2nc(-c4cccc5ccccc45)nc(-c4cccc5ccccc45)n2)c13 ; c1ccc2cc3c(cc2c1)oc1ccc(-c2nc(-c4cccc5ccccc45)nc(-c4cccc5ccccc45)n2)cc13 ; c1ccc2cc3c(cc2c1)oc1cc(-c2nc(-c4cccc5ccccc45)nc(-c4cccc5ccccc45)n2)ccc13 ; c1ccc2cc3c(cc2c1)oc1c(-c2nc(-c4cccc5ccccc45)nc(-c4cccc5ccccc45)n2)cccc13]

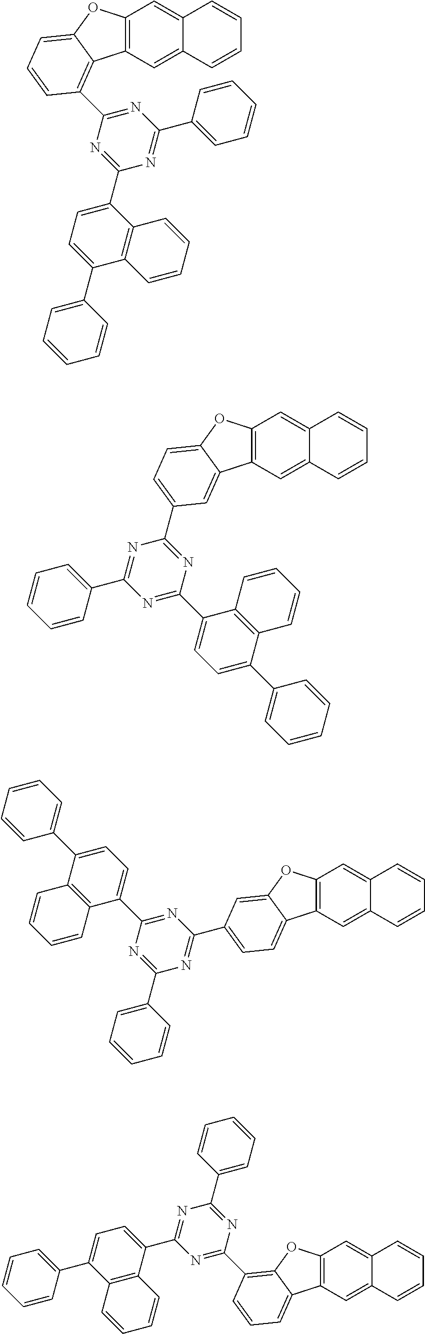 [SMILES: c1ccc(-c2nc(-c3ccc(-c4ccccc4)c4ccccc34)nc(-c3cccc4oc5cc6ccccc6cc5c34)n2)cc1 ; c1ccc(-c2nc(-c3ccc4oc5cc6ccccc6cc5c4c3)nc(-c3ccc(-c4ccccc4)c4ccccc34)n2)cc1 ; c1ccc(-c2nc(-c3ccc4c(c3)oc3cc5ccccc5cc34)nc(-c3ccc(-c4ccccc4)c4ccccc34)n2)cc1 ; c1ccc(-c2nc(-c3ccc(-c4ccccc4)c4ccccc34)nc(-c3cccc4c3oc3cc5ccccc5cc34)n2)cc1]

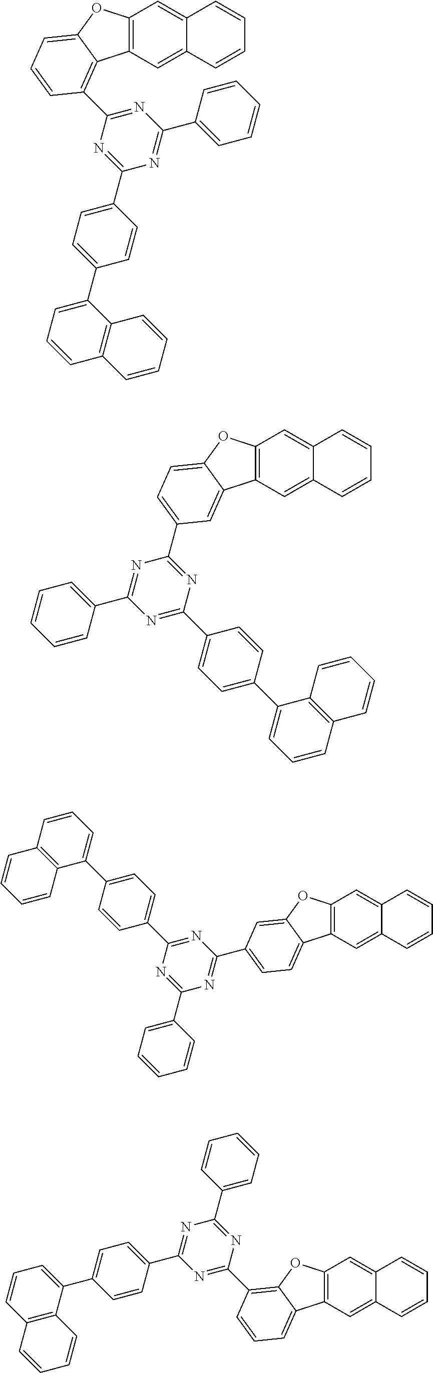 [SMILES: c1ccc(-c2nc(-c3ccc(-c4cccc5ccccc45)cc3)nc(-c3cccc4oc5cc6ccccc6cc5c34)n2)cc1 ; c1ccc(-c2nc(-c3ccc(-c4cccc5ccccc45)cc3)nc(-c3ccc4oc5cc6ccccc6cc5c4c3)n2)cc1 ; c1ccc(-c2nc(-c3ccc(-c4cccc5ccccc45)cc3)nc(-c3ccc4c(c3)oc3cc5ccccc5cc34)n2)cc1 ; c1ccc(-c2nc(-c3ccc(-c4cccc5ccccc45)cc3)nc(-c3cccc4c3oc3cc5ccccc5cc34)n2)cc1]

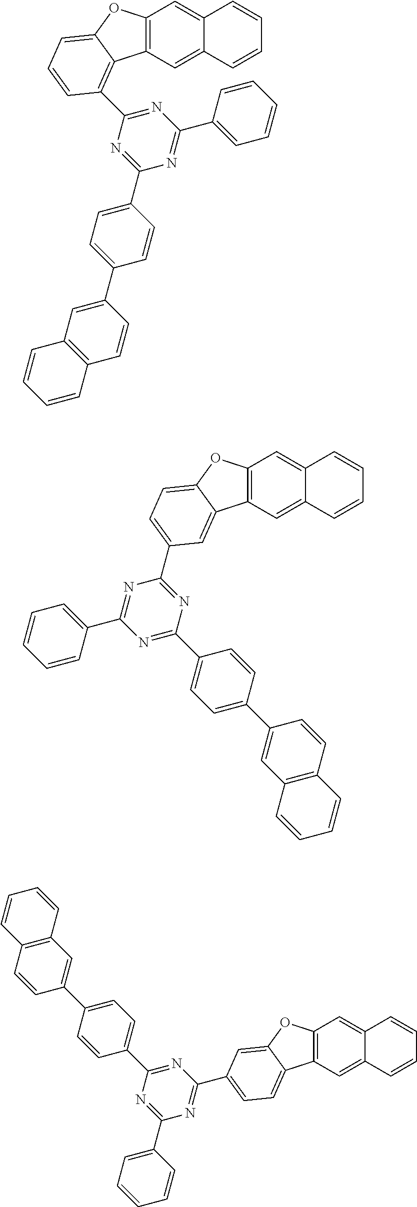 [SMILES: c1ccc(-c2nc(-c3ccc(-c4ccc5ccccc5c4)cc3)nc(-c3cccc4oc5cc6ccccc6cc5c34)n2)cc1 ; c1ccc(-c2nc(-c3ccc(-c4ccc5ccccc5c4)cc3)nc(-c3ccc4oc5cc6ccccc6cc5c4c3)n2)cc1 ; c1ccc(-c2nc(-c3ccc(-c4ccc5ccccc5c4)cc3)nc(-c3ccc4c(c3)oc3cc5ccccc5cc34)n2)cc1]

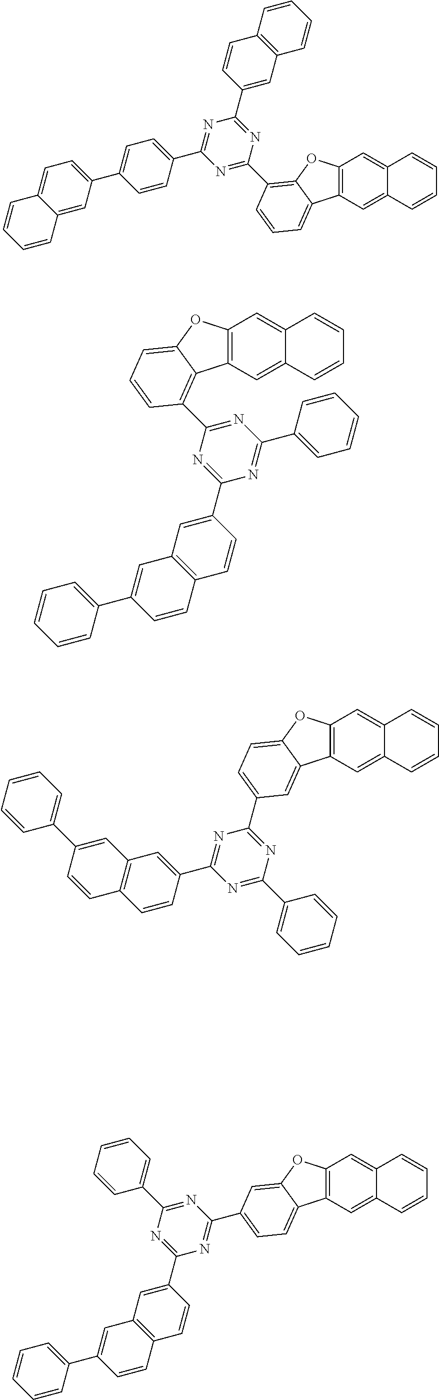 [SMILES: c1ccc2cc(-c3ccc(-c4nc(-c5ccc6ccccc6c5)nc(-c5cccc6c5oc5cc7ccccc7cc56)n4)cc3)ccc2c1 ; c1ccc(-c2ccc3ccc(-c4nc(-c5ccccc5)nc(-c5cccc6oc7cc8ccccc8cc7c56)n4)cc3c2)cc1 ; c1ccc(-c2ccc3ccc(-c4nc(-c5ccccc5)nc(-c5ccc6oc7cc8ccccc8cc7c6c5)n4)cc3c2)cc1 ; c1ccc(-c2ccc3ccc(-c4nc(-c5ccccc5)nc(-c5ccc6c(c5)oc5cc7ccccc7cc56)n4)cc3c2)cc1]

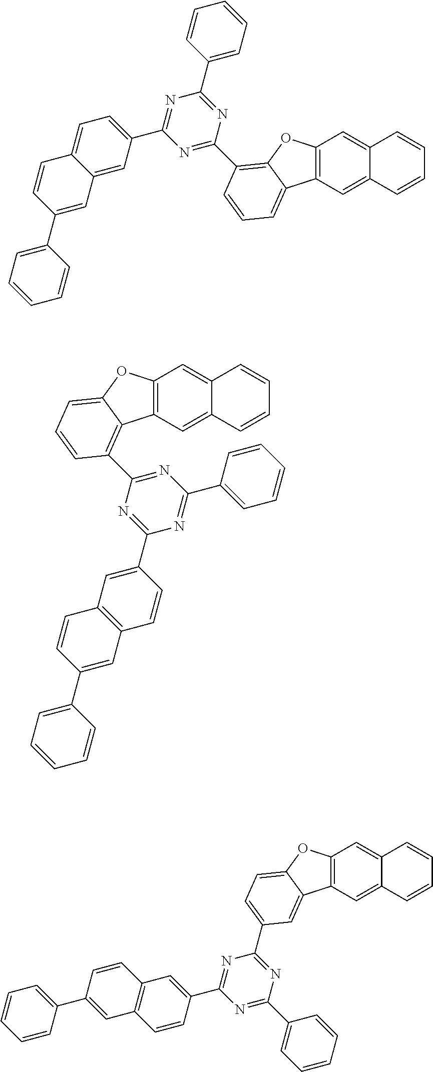 [SMILES: c1ccc(-c2ccc3ccc(-c4nc(-c5ccccc5)nc(-c5cccc6c5oc5cc7ccccc7cc56)n4)cc3c2)cc1 ; c1ccc(-c2ccc3cc(-c4nc(-c5ccccc5)nc(-c5cccc6oc7cc8ccccc8cc7c56)n4)ccc3c2)cc1 ; c1ccc(-c2ccc3cc(-c4nc(-c5ccccc5)nc(-c5ccc6oc7cc8ccccc8cc7c6c5)n4)ccc3c2)cc1]

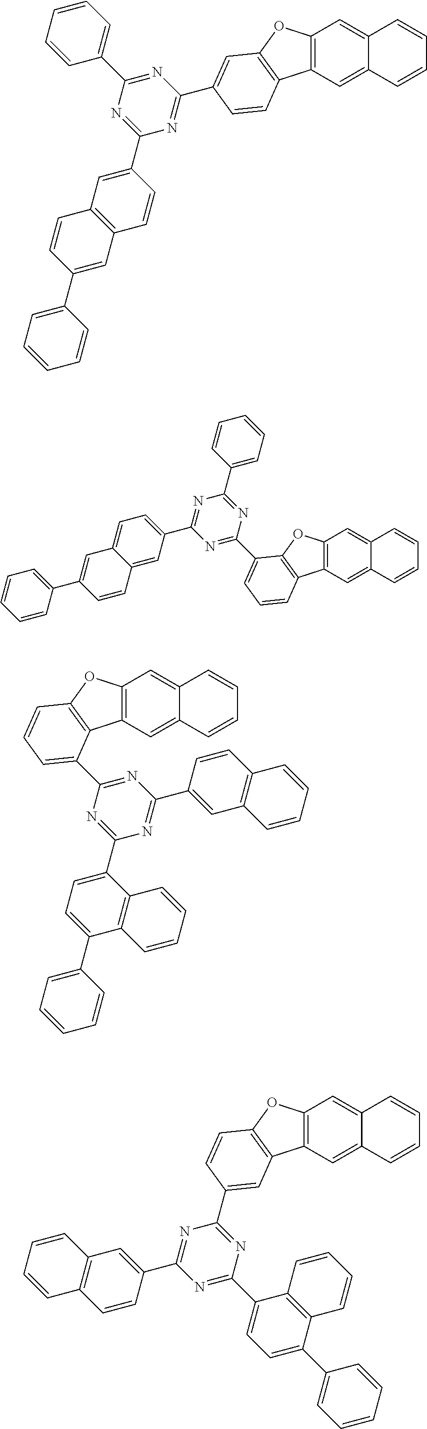 [SMILES: c1ccc(-c2ccc3cc(-c4nc(-c5ccccc5)nc(-c5ccc6c(c5)oc5cc7ccccc7cc56)n4)ccc3c2)cc1 ; c1ccc(-c2ccc3cc(-c4nc(-c5ccccc5)nc(-c5cccc6c5oc5cc7ccccc7cc56)n4)ccc3c2)cc1 ; c1ccc(-c2ccc(-c3nc(-c4ccc5ccccc5c4)nc(-c4cccc5oc6cc7ccccc7cc6c45)n3)c3ccccc23)cc1 ; c1ccc(-c2ccc(-c3nc(-c4ccc5ccccc5c4)nc(-c4ccc5oc6cc7ccccc7cc6c5c4)n3)c3ccccc23)cc1]

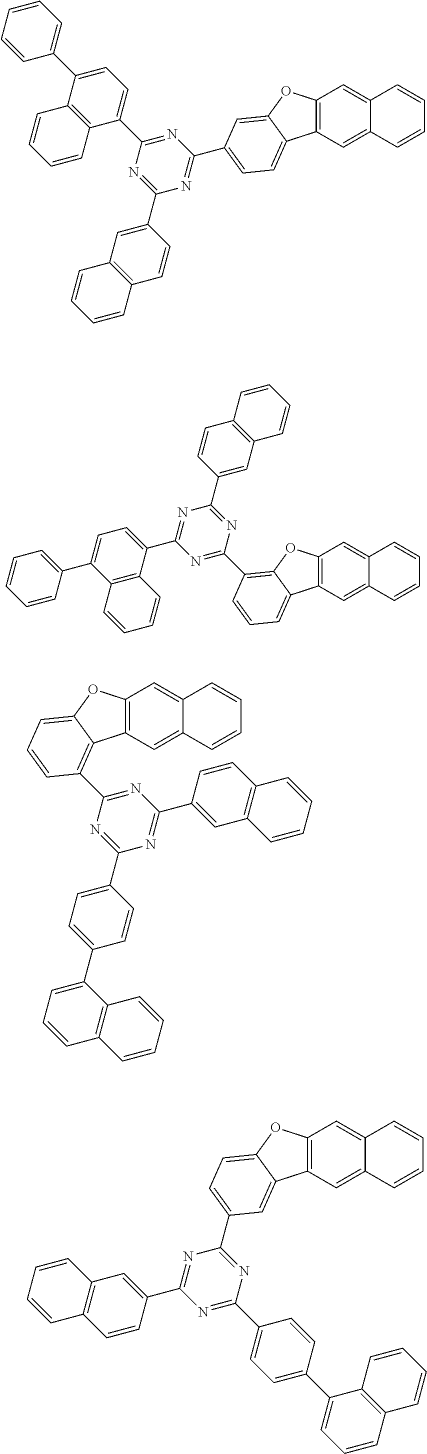 [SMILES: c1ccc(-c2ccc(-c3nc(-c4ccc5ccccc5c4)nc(-c4ccc5c(c4)oc4cc6ccccc6cc45)n3)c3ccccc23)cc1 ; c1ccc(-c2ccc(-c3nc(-c4ccc5ccccc5c4)nc(-c4cccc5c4oc4cc6ccccc6cc45)n3)c3ccccc23)cc1 ; c1ccc2cc(-c3nc(-c4ccc(-c5cccc6ccccc56)cc4)nc(-c4cccc5oc6cc7ccccc7cc6c45)n3)ccc2c1 ; c1ccc2cc(-c3nc(-c4ccc(-c5cccc6ccccc56)cc4)nc(-c4ccc5oc6cc7ccccc7cc6c5c4)n3)ccc2c1]

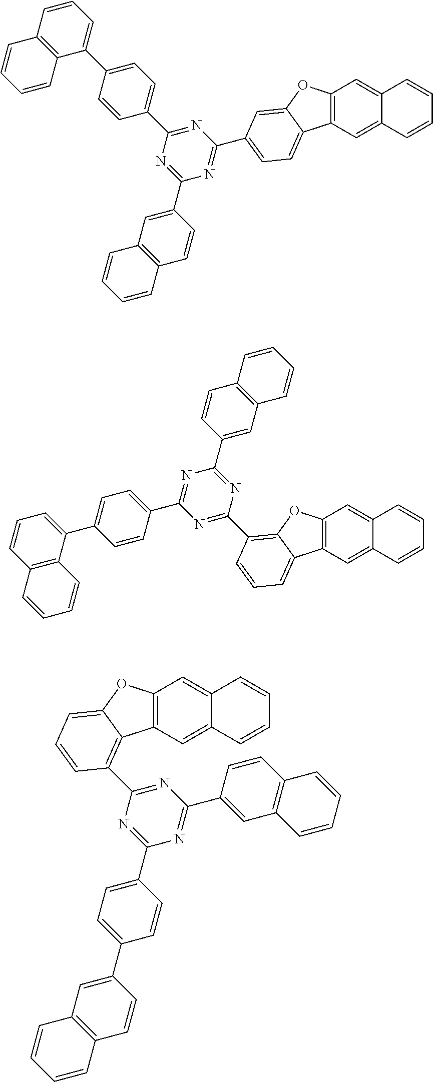 [SMILES: c1ccc2cc(-c3nc(-c4ccc(-c5cccc6ccccc56)cc4)nc(-c4ccc5c(c4)oc4cc6ccccc6cc45)n3)ccc2c1 ; c1ccc2cc(-c3nc(-c4ccc(-c5cccc6ccccc56)cc4)nc(-c4cccc5c4oc4cc6ccccc6cc45)n3)ccc2c1 ; c1ccc2cc(-c3ccc(-c4nc(-c5ccc6ccccc6c5)nc(-c5cccc6oc7cc8ccccc8cc7c56)n4)cc3)ccc2c1]

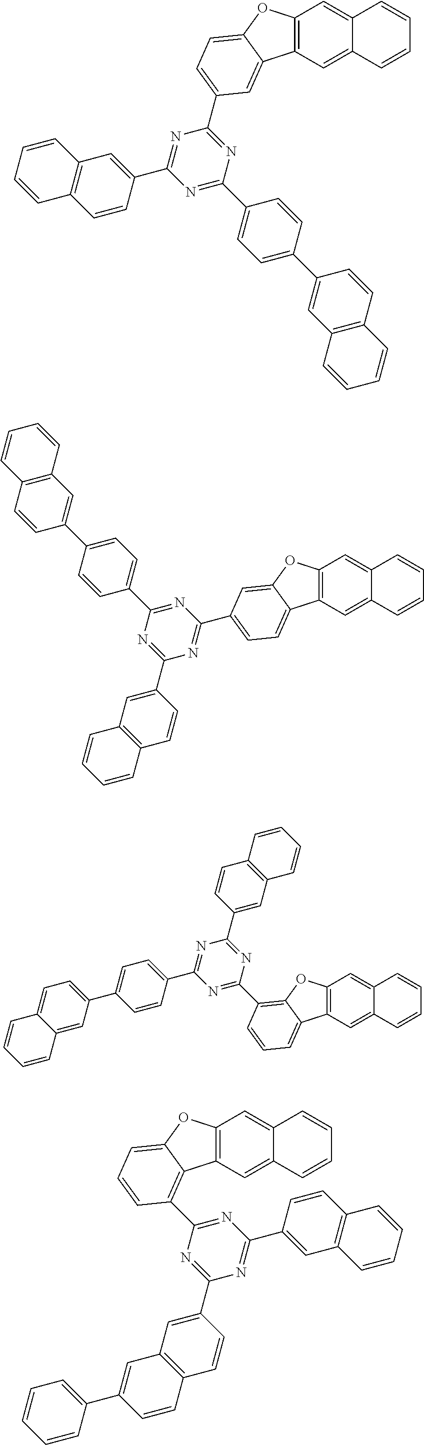 [SMILES: c1ccc2cc(-c3ccc(-c4nc(-c5ccc6ccccc6c5)nc(-c5ccc6oc7cc8ccccc8cc7c6c5)n4)cc3)ccc2c1 ; c1ccc2cc(-c3ccc(-c4nc(-c5ccc6ccccc6c5)nc(-c5ccc6c(c5)oc5cc7ccccc7cc56)n4)cc3)ccc2c1 ; c1ccc2cc(-c3ccc(-c4nc(-c5ccc6ccccc6c5)nc(-c5cccc6c5oc5cc7ccccc7cc56)n4)cc3)ccc2c1 ; c1ccc(-c2ccc3ccc(-c4nc(-c5ccc6ccccc6c5)nc(-c5cccc6oc7cc8ccccc8cc7c56)n4)cc3c2)cc1]

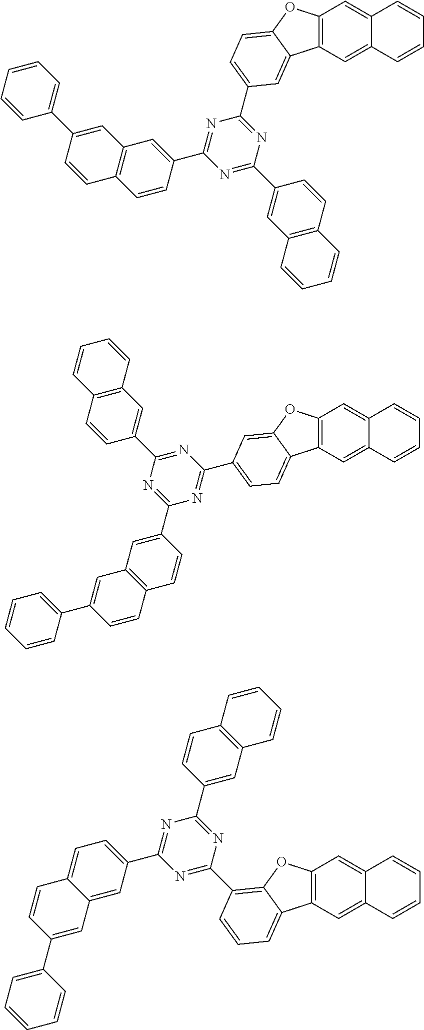 [SMILES: c1ccc(-c2ccc3ccc(-c4nc(-c5ccc6ccccc6c5)nc(-c5ccc6oc7cc8ccccc8cc7c6c5)n4)cc3c2)cc1 ; c1ccc(-c2ccc3ccc(-c4nc(-c5ccc6ccccc6c5)nc(-c5ccc6c(c5)oc5cc7ccccc7cc56)n4)cc3c2)cc1 ; c1ccc(-c2ccc3ccc(-c4nc(-c5ccc6ccccc6c5)nc(-c5cccc6c5oc5cc7ccccc7cc56)n4)cc3c2)cc1]

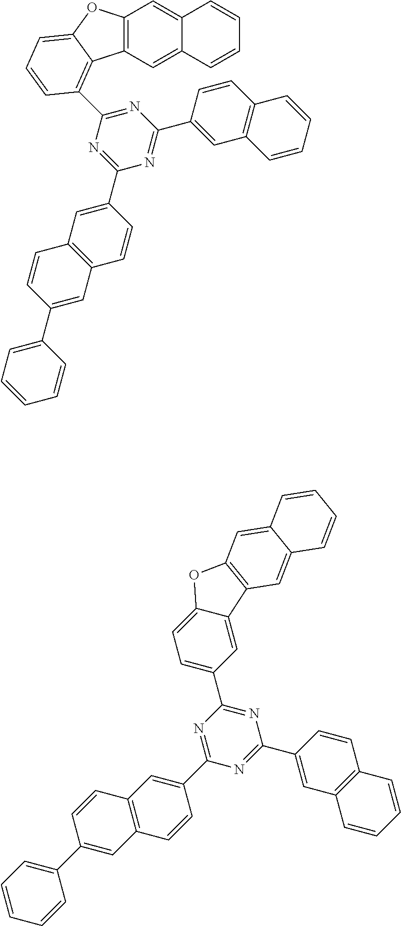 [SMILES: c1ccc(-c2ccc3cc(-c4nc(-c5ccc6ccccc6c5)nc(-c5cccc6oc7cc8ccccc8cc7c56)n4)ccc3c2)cc1 ; c1ccc(-c2ccc3cc(-c4nc(-c5ccc6ccccc6c5)nc(-c5ccc6oc7cc8ccccc8cc7c6c5)n4)ccc3c2)cc1]

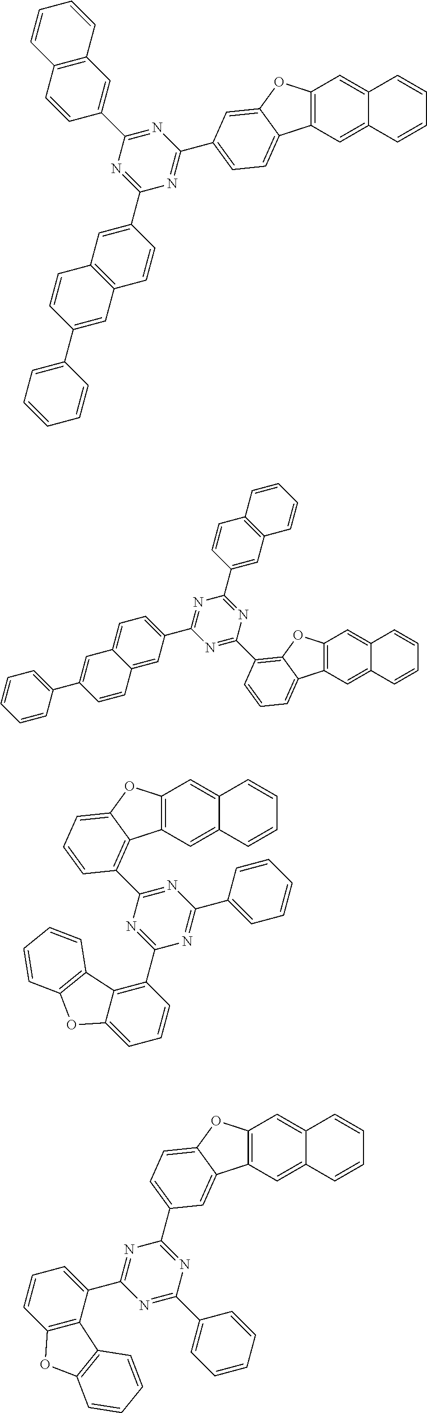 [SMILES: c1ccc(-c2ccc3cc(-c4nc(-c5ccc6ccccc6c5)nc(-c5ccc6c(c5)oc5cc7ccccc7cc56)n4)ccc3c2)cc1 ; c1ccc(-c2ccc3cc(-c4nc(-c5ccc6ccccc6c5)nc(-c5cccc6c5oc5cc7ccccc7cc56)n4)ccc3c2)cc1 ; c1ccc(-c2nc(-c3cccc4oc5ccccc5c34)nc(-c3cccc4oc5cc6ccccc6cc5c34)n2)cc1 ; c1ccc(-c2nc(-c3ccc4oc5cc6ccccc6cc5c4c3)nc(-c3cccc4oc5ccccc5c34)n2)cc1]

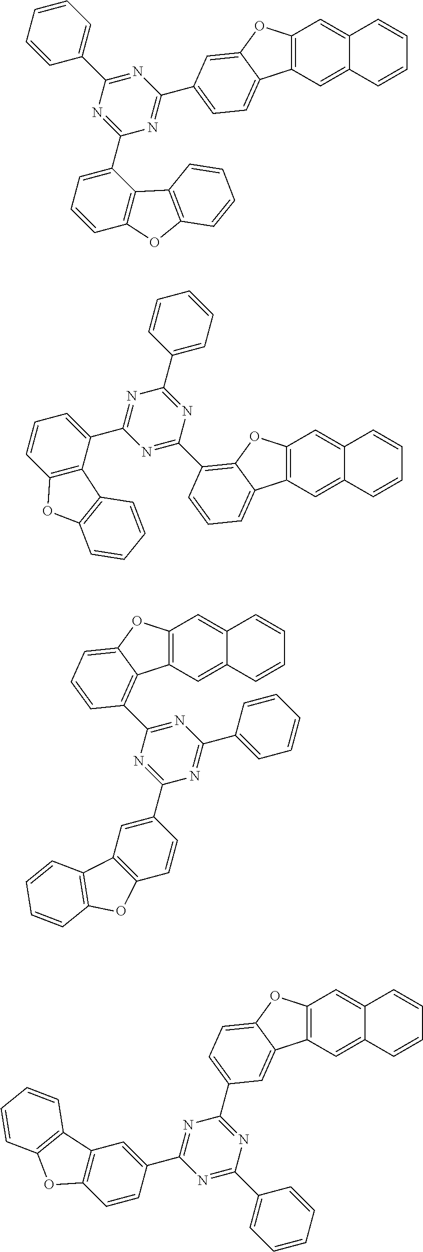 [SMILES: c1ccc(-c2nc(-c3ccc4c(c3)oc3cc5ccccc5cc34)nc(-c3cccc4oc5ccccc5c34)n2)cc1 ; c1ccc(-c2nc(-c3cccc4c3oc3cc5ccccc5cc34)nc(-c3cccc4oc5ccccc5c34)n2)cc1 ; c1ccc(-c2nc(-c3ccc4oc5ccccc5c4c3)nc(-c3cccc4oc5cc6ccccc6cc5c34)n2)cc1 ; c1ccc(-c2nc(-c3ccc4oc5ccccc5c4c3)nc(-c3ccc4oc5cc6ccccc6cc5c4c3)n2)cc1]

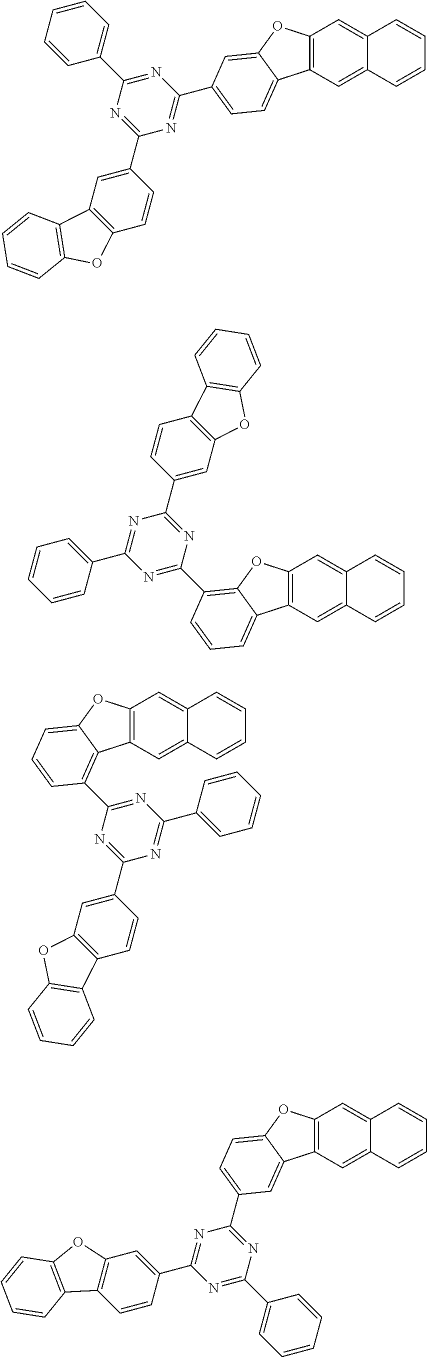

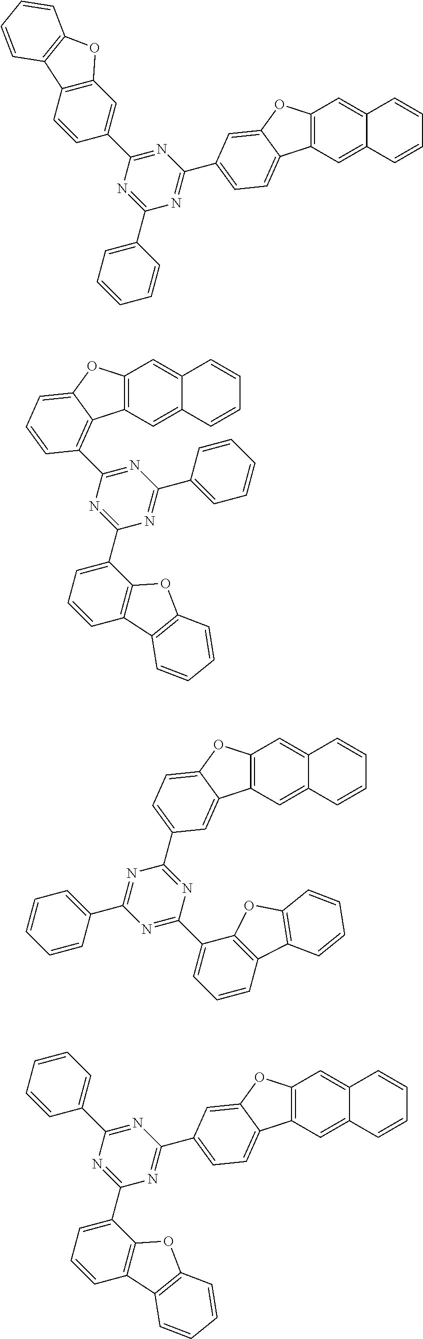 [SMILES: c1ccc(-c2nc(-c3ccc4c(c3)oc3ccccc34)nc(-c3ccc4c(c3)oc3cc5ccccc5cc34)n2)cc1 ; c1ccc(-c2nc(-c3cccc4c3oc3ccccc34)nc(-c3cccc4oc5cc6ccccc6cc5c34)n2)cc1 ; c1ccc(-c2nc(-c3ccc4oc5cc6ccccc6cc5c4c3)nc(-c3cccc4c3oc3ccccc34)n2)cc1 ; c1ccc(-c2nc(-c3ccc4c(c3)oc3cc5ccccc5cc34)nc(-c3cccc4c3oc3ccccc34)n2)cc1]

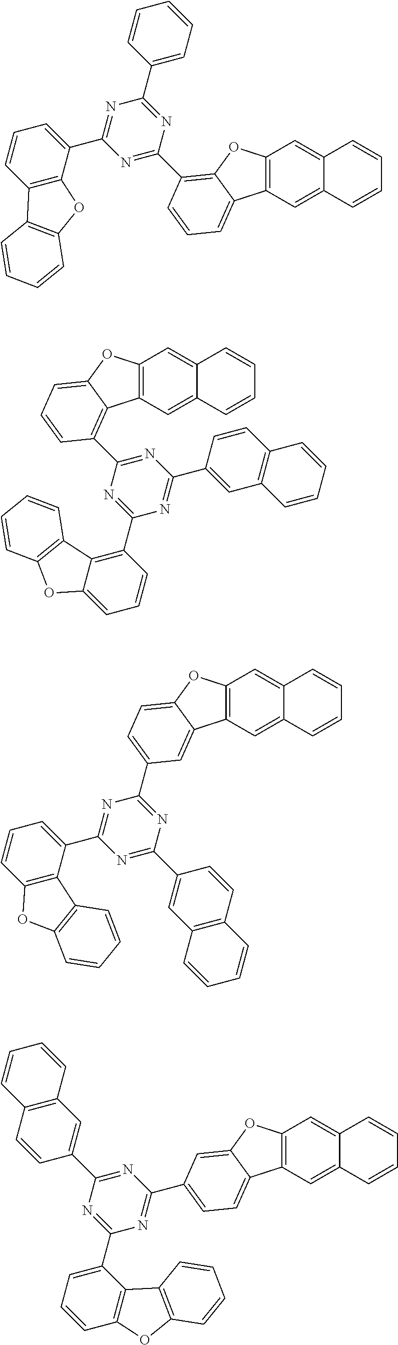 [SMILES: c1ccc(-c2nc(-c3cccc4c3oc3ccccc34)nc(-c3cccc4c3oc3cc5ccccc5cc34)n2)cc1 ; c1ccc2cc(-c3nc(-c4cccc5oc6ccccc6c45)nc(-c4cccc5oc6cc7ccccc7cc6c45)n3)ccc2c1 ; c1ccc2cc(-c3nc(-c4ccc5oc6cc7ccccc7cc6c5c4)nc(-c4cccc5oc6ccccc6c45)n3)ccc2c1 ; c1ccc2cc(-c3nc(-c4ccc5c(c4)oc4cc6ccccc6cc45)nc(-c4cccc5oc6ccccc6c45)n3)ccc2c1]

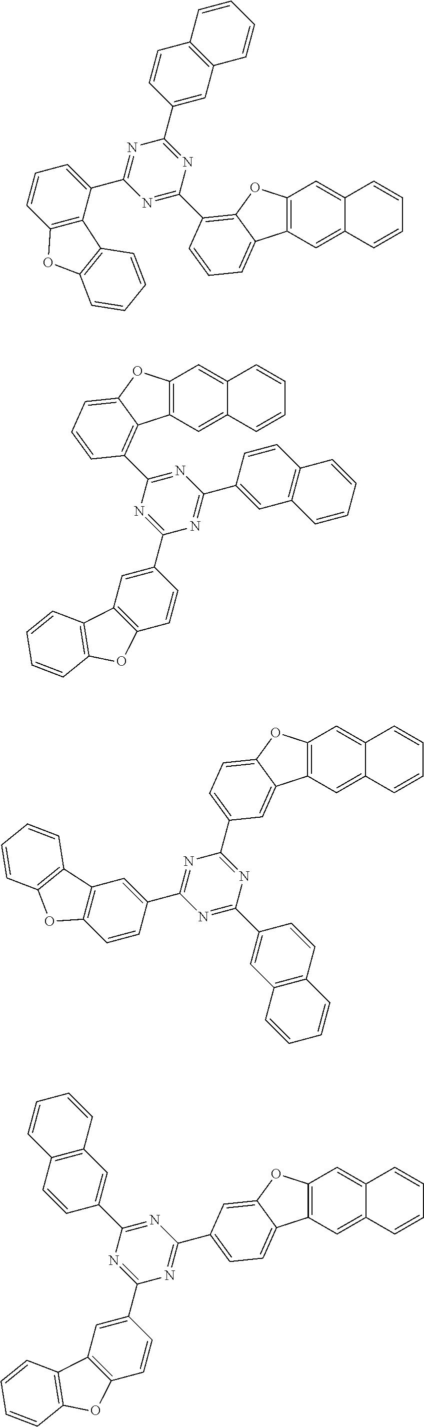 [SMILES: c1ccc2cc(-c3nc(-c4cccc5c4oc4cc6ccccc6cc45)nc(-c4cccc5oc6ccccc6c45)n3)ccc2c1 ; c1ccc2cc(-c3nc(-c4ccc5oc6ccccc6c5c4)nc(-c4cccc5oc6cc7ccccc7cc6c45)n3)ccc2c1 ; c1ccc2cc(-c3nc(-c4ccc5oc6ccccc6c5c4)nc(-c4ccc5oc6cc7ccccc7cc6c5c4)n3)ccc2c1 ; c1ccc2cc(-c3nc(-c4ccc5c(c4)oc4cc6ccccc6cc45)nc(-c4ccc5oc6ccccc6c5c4)n3)ccc2c1]

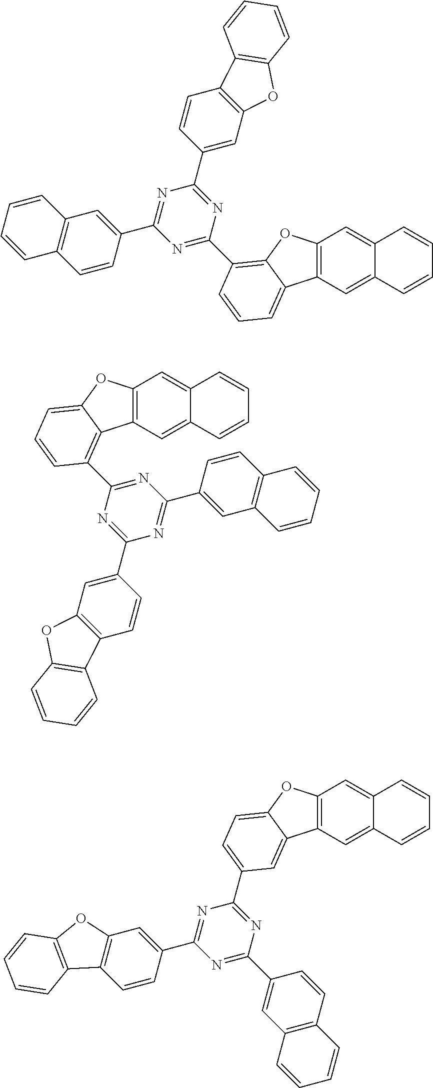 [SMILES: c1ccc2cc(-c3nc(-c4ccc5c(c4)oc4ccccc45)nc(-c4cccc5c4oc4cc6ccccc6cc45)n3)ccc2c1 ; c1ccc2cc(-c3nc(-c4ccc5c(c4)oc4ccccc45)nc(-c4cccc5oc6cc7ccccc7cc6c45)n3)ccc2c1 ; c1ccc2cc(-c3nc(-c4ccc5c(c4)oc4ccccc45)nc(-c4ccc5oc6cc7ccccc7cc6c5c4)n3)ccc2c1]

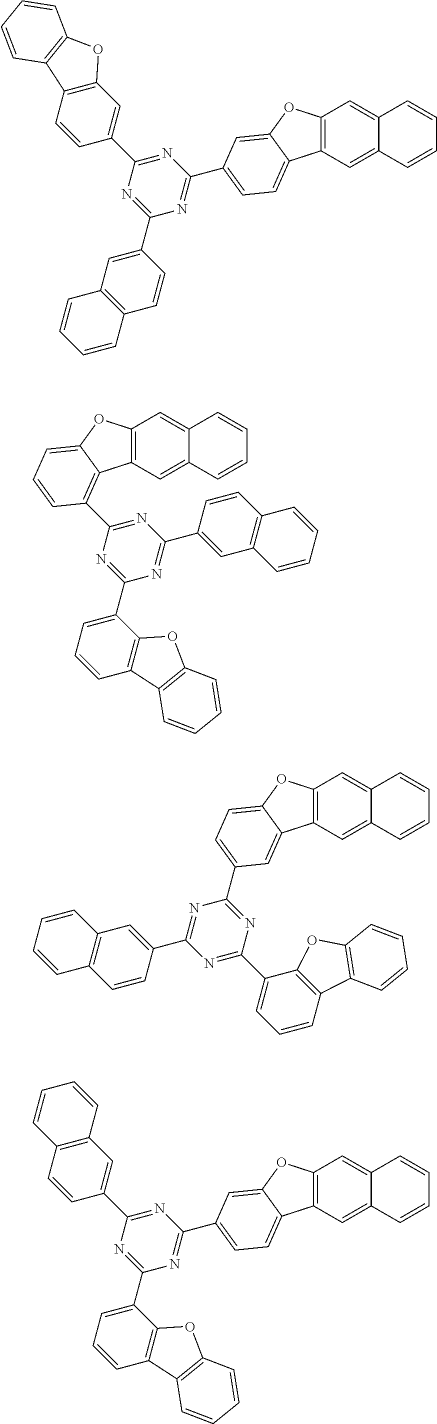 [SMILES: c1ccc2cc(-c3nc(-c4ccc5c(c4)oc4ccccc45)nc(-c4ccc5c(c4)oc4cc6ccccc6cc45)n3)ccc2c1 ; c1ccc2cc(-c3nc(-c4cccc5c4oc4ccccc45)nc(-c4cccc5oc6cc7ccccc7cc6c45)n3)ccc2c1 ; c1ccc2cc(-c3nc(-c4ccc5oc6cc7ccccc7cc6c5c4)nc(-c4cccc5c4oc4ccccc45)n3)ccc2c1 ; c1ccc2cc(-c3nc(-c4ccc5c(c4)oc4cc6ccccc6cc45)nc(-c4cccc5c4oc4ccccc45)n3)ccc2c1]

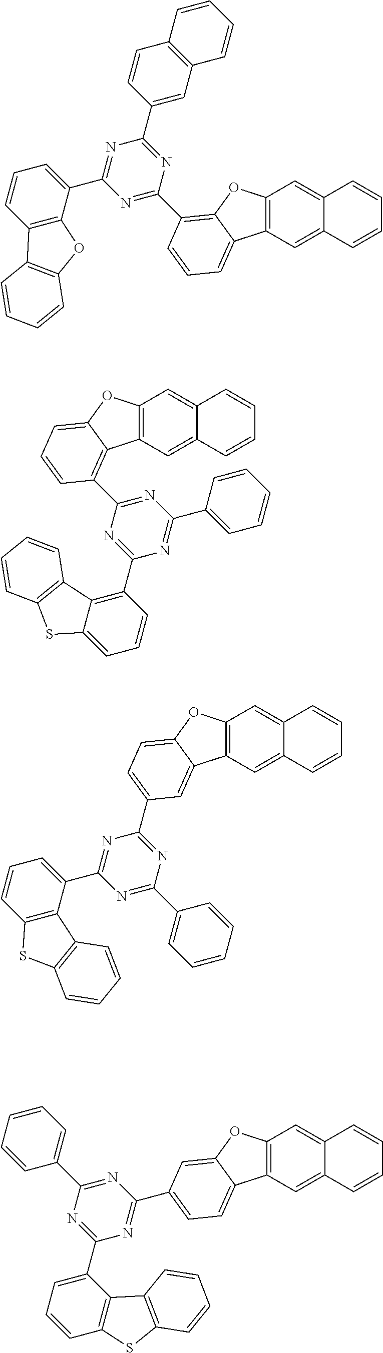 [SMILES: c1ccc2cc(-c3nc(-c4cccc5c4oc4ccccc45)nc(-c4cccc5c4oc4cc6ccccc6cc45)n3)ccc2c1 ; c1ccc(-c2nc(-c3cccc4oc5cc6ccccc6cc5c34)nc(-c3cccc4sc5ccccc5c34)n2)cc1 ; c1ccc(-c2nc(-c3ccc4oc5cc6ccccc6cc5c4c3)nc(-c3cccc4sc5ccccc5c34)n2)cc1 ; c1ccc(-c2nc(-c3ccc4c(c3)oc3cc5ccccc5cc34)nc(-c3cccc4sc5ccccc5c34)n2)cc1]

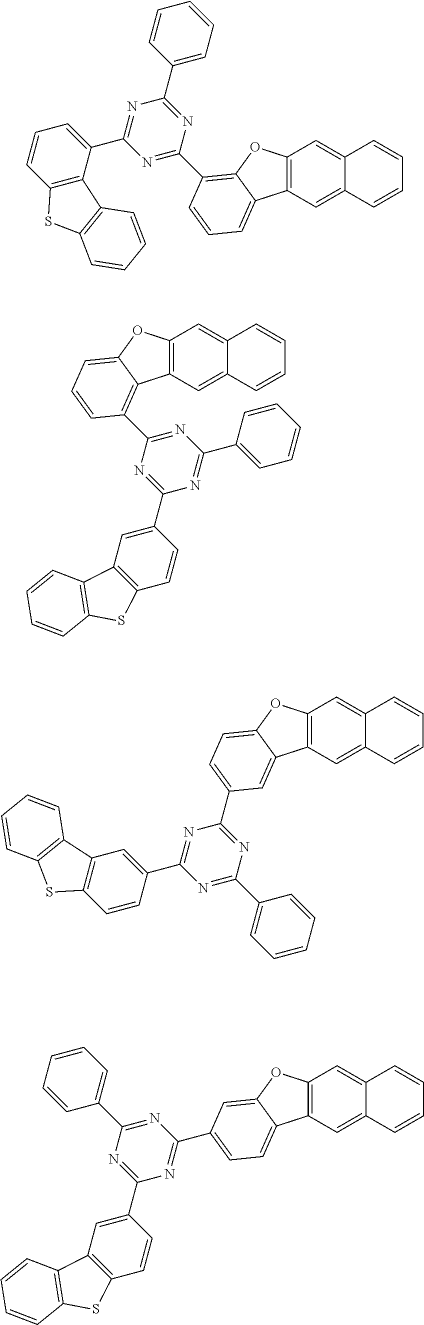 [SMILES: c1ccc(-c2nc(-c3cccc4c3oc3cc5ccccc5cc34)nc(-c3cccc4sc5ccccc5c34)n2)cc1 ; c1ccc(-c2nc(-c3ccc4sc5ccccc5c4c3)nc(-c3cccc4oc5cc6ccccc6cc5c34)n2)cc1 ; c1ccc(-c2nc(-c3ccc4oc5cc6ccccc6cc5c4c3)nc(-c3ccc4sc5ccccc5c4c3)n2)cc1 ; c1ccc(-c2nc(-c3ccc4c(c3)oc3cc5ccccc5cc34)nc(-c3ccc4sc5ccccc5c4c3)n2)cc1]

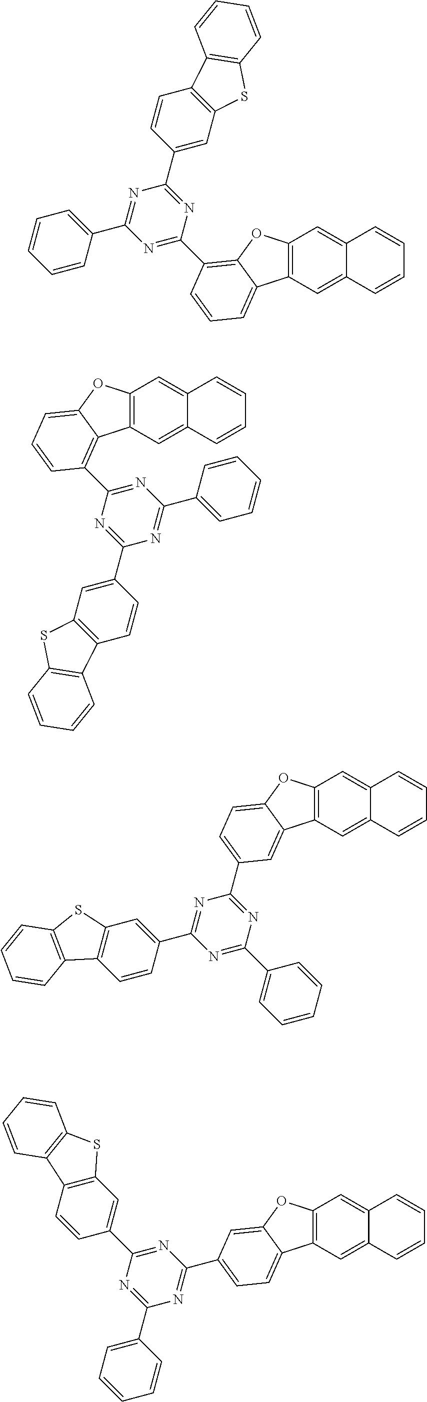 [SMILES: c1ccc(-c2nc(-c3ccc4c(c3)sc3ccccc34)nc(-c3cccc4c3oc3cc5ccccc5cc34)n2)cc1 ; c1ccc(-c2nc(-c3ccc4c(c3)sc3ccccc34)nc(-c3cccc4oc5cc6ccccc6cc5c34)n2)cc1 ; c1ccc(-c2nc(-c3ccc4c(c3)sc3ccccc34)nc(-c3ccc4oc5cc6ccccc6cc5c4c3)n2)cc1 ; c1ccc(-c2nc(-c3ccc4c(c3)oc3cc5ccccc5cc34)nc(-c3ccc4c(c3)sc3ccccc34)n2)cc1]

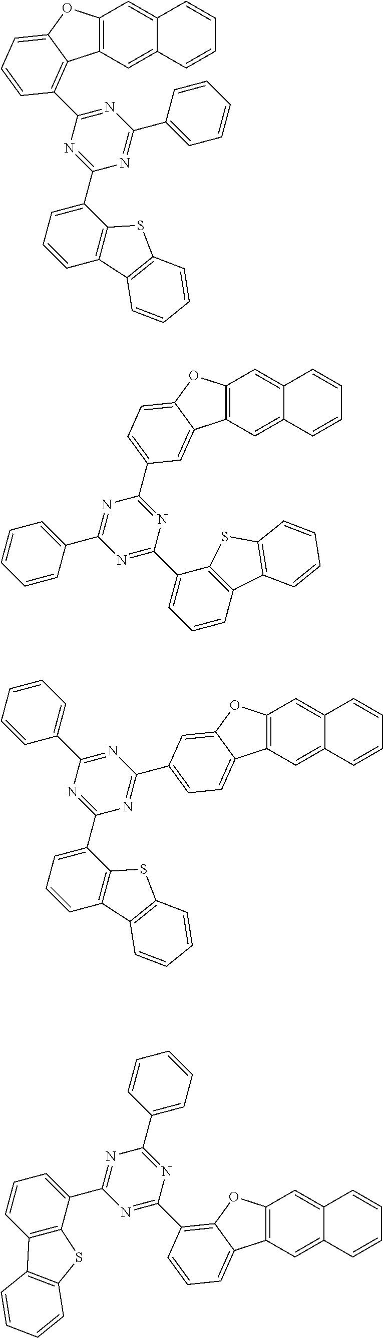 [SMILES: c1ccc(-c2nc(-c3cccc4c3sc3ccccc34)nc(-c3cccc4oc5cc6ccccc6cc5c34)n2)cc1 ; c1ccc(-c2nc(-c3ccc4oc5cc6ccccc6cc5c4c3)nc(-c3cccc4c3sc3ccccc34)n2)cc1 ; c1ccc(-c2nc(-c3ccc4c(c3)oc3cc5ccccc5cc34)nc(-c3cccc4c3sc3ccccc34)n2)cc1 ; c1ccc(-c2nc(-c3cccc4c3oc3cc5ccccc5cc34)nc(-c3cccc4c3sc3ccccc34)n2)cc1]

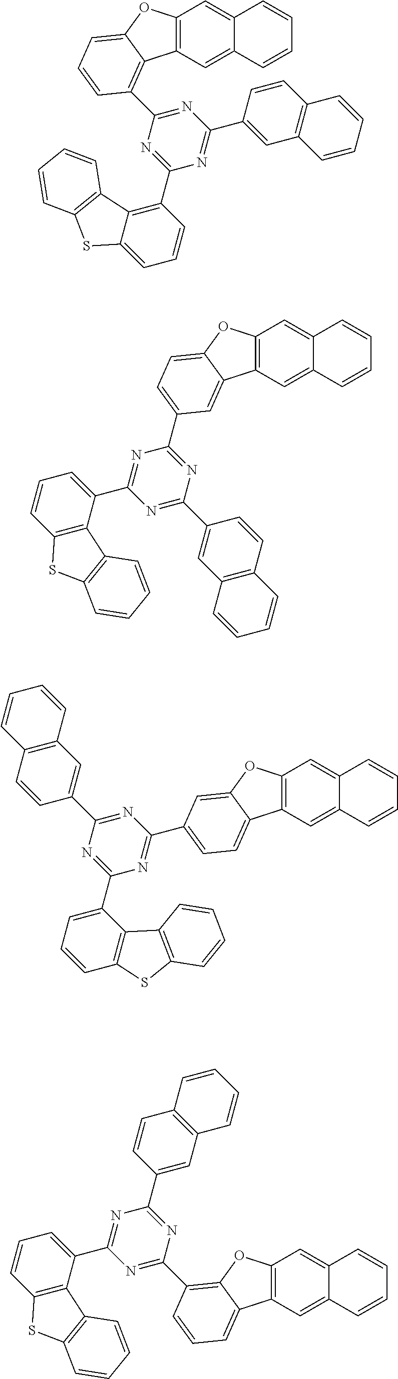 [SMILES: c1ccc2cc(-c3nc(-c4cccc5oc6cc7ccccc7cc6c45)nc(-c4cccc5sc6ccccc6c45)n3)ccc2c1 ; c1ccc2cc(-c3nc(-c4ccc5oc6cc7ccccc7cc6c5c4)nc(-c4cccc5sc6ccccc6c45)n3)ccc2c1 ; c1ccc2cc(-c3nc(-c4ccc5c(c4)oc4cc6ccccc6cc45)nc(-c4cccc5sc6ccccc6c45)n3)ccc2c1 ; c1ccc2cc(-c3nc(-c4cccc5c4oc4cc6ccccc6cc45)nc(-c4cccc5sc6ccccc6c45)n3)ccc2c1]

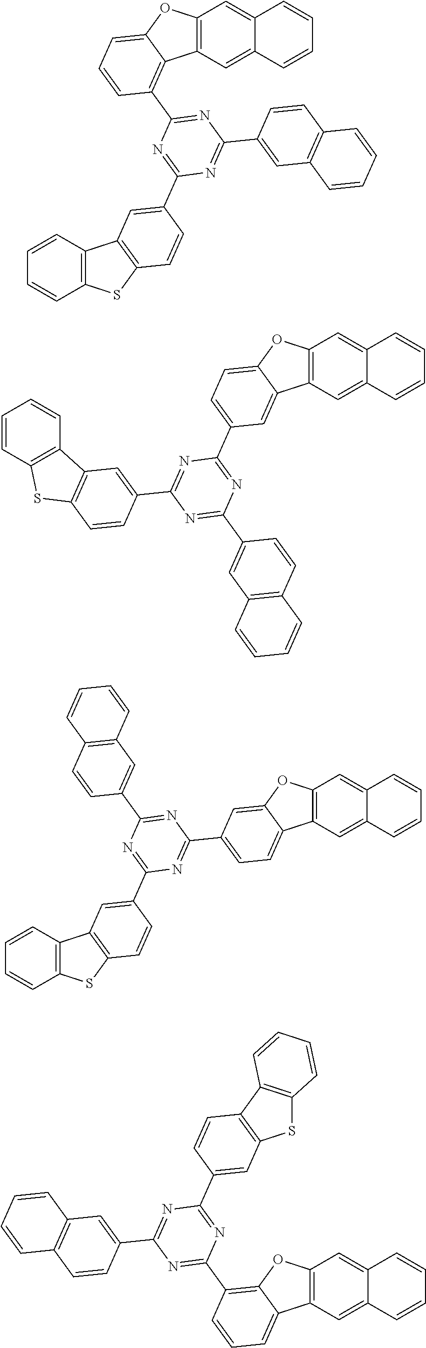 [SMILES: c1ccc2cc(-c3nc(-c4ccc5sc6ccccc6c5c4)nc(-c4cccc5oc6cc7ccccc7cc6c45)n3)ccc2c1 ; c1ccc2cc(-c3nc(-c4ccc5oc6cc7ccccc7cc6c5c4)nc(-c4ccc5sc6ccccc6c5c4)n3)ccc2c1 ; c1ccc2cc(-c3nc(-c4ccc5c(c4)oc4cc6ccccc6cc45)nc(-c4ccc5sc6ccccc6c5c4)n3)ccc2c1 ; c1ccc2cc(-c3nc(-c4ccc5c(c4)sc4ccccc45)nc(-c4cccc5c4oc4cc6ccccc6cc45)n3)ccc2c1]

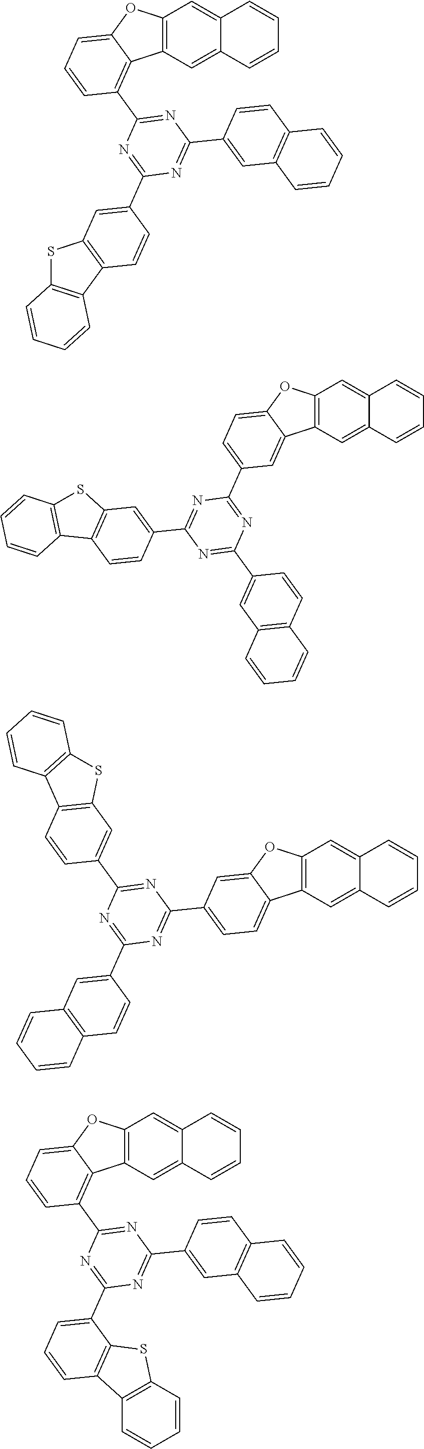 [SMILES: c1ccc2cc(-c3nc(-c4ccc5c(c4)sc4ccccc45)nc(-c4cccc5oc6cc7ccccc7cc6c45)n3)ccc2c1 ; c1ccc2cc(-c3nc(-c4ccc5c(c4)sc4ccccc45)nc(-c4ccc5oc6cc7ccccc7cc6c5c4)n3)ccc2c1 ; c1ccc2cc(-c3nc(-c4ccc5c(c4)oc4cc6ccccc6cc45)nc(-c4ccc5c(c4)sc4ccccc45)n3)ccc2c1 ; c1ccc2cc(-c3nc(-c4cccc5c4sc4ccccc45)nc(-c4cccc5oc6cc7ccccc7cc6c45)n3)ccc2c1]

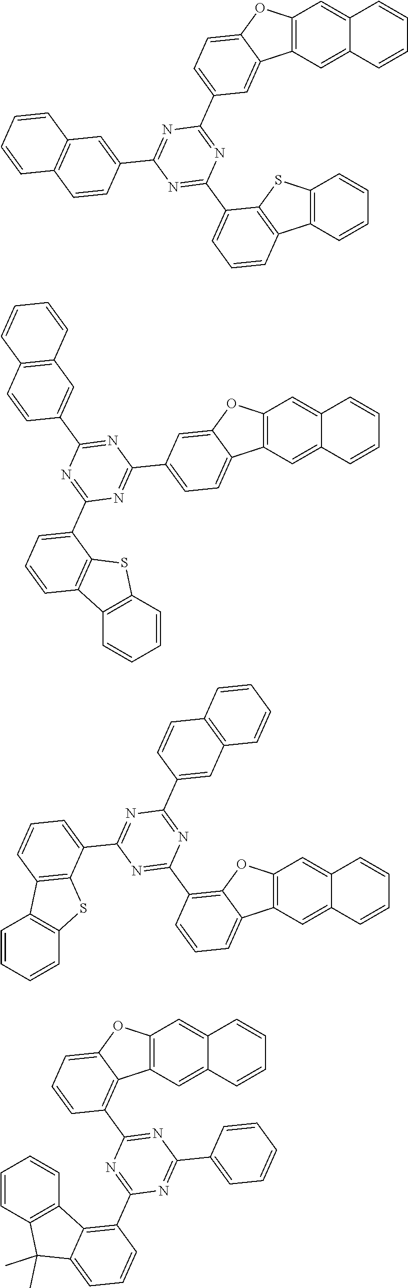 [SMILES: c1ccc2cc(-c3nc(-c4ccc5oc6cc7ccccc7cc6c5c4)nc(-c4cccc5c4sc4ccccc45)n3)ccc2c1 ; c1ccc2cc(-c3nc(-c4ccc5c(c4)oc4cc6ccccc6cc45)nc(-c4cccc5c4sc4ccccc45)n3)ccc2c1 ; c1ccc2cc(-c3nc(-c4cccc5c4oc4cc6ccccc6cc45)nc(-c4cccc5c4sc4ccccc45)n3)ccc2c1 ; CC1(C)c2ccccc2-c2c(-c3nc(-c4ccccc4)nc(-c4cccc5oc6cc7ccccc7cc6c45)n3)cccc21]

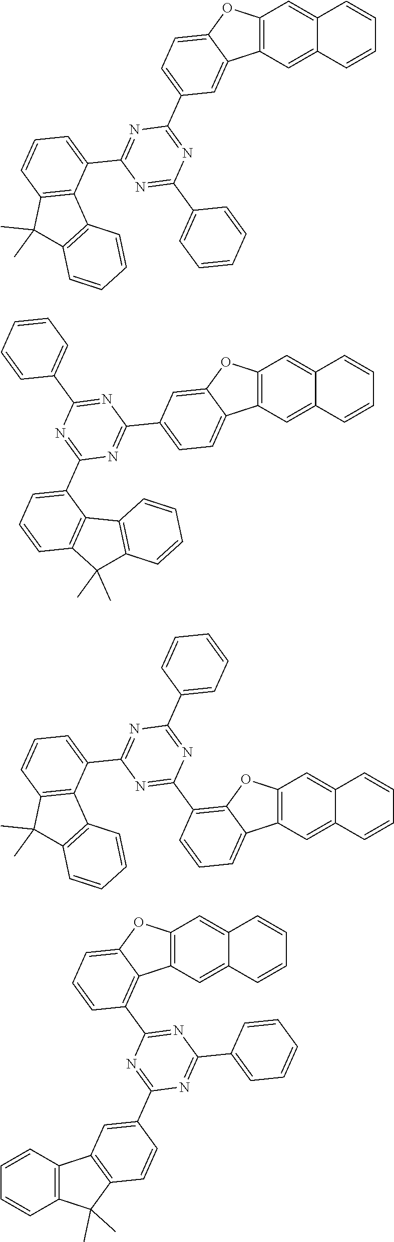

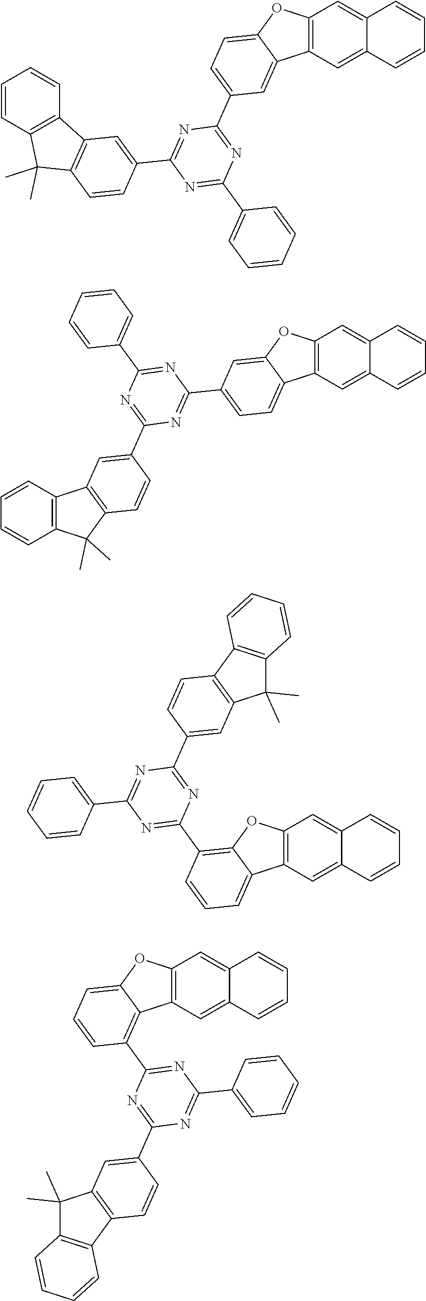

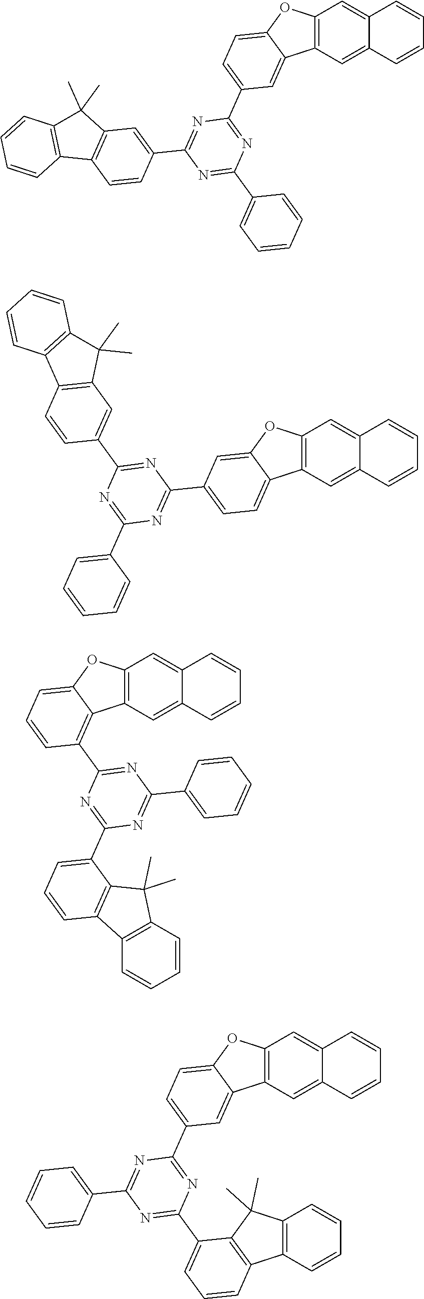

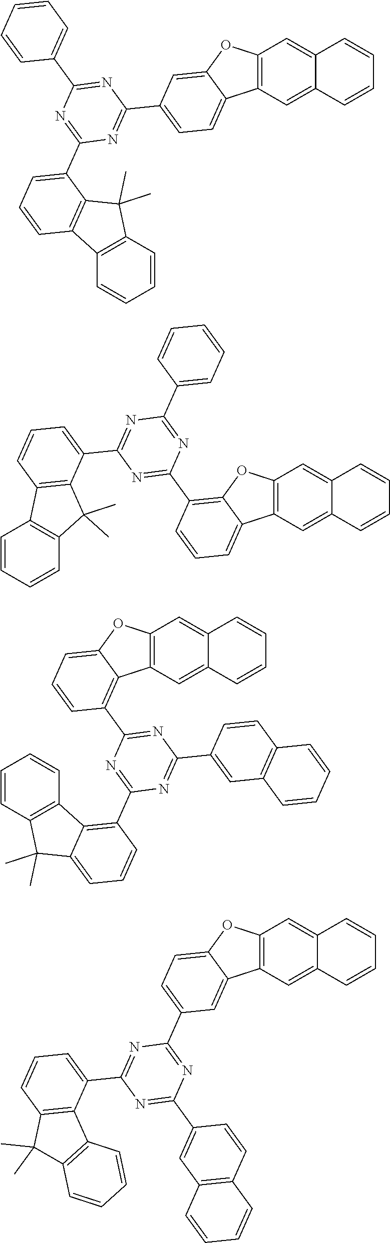

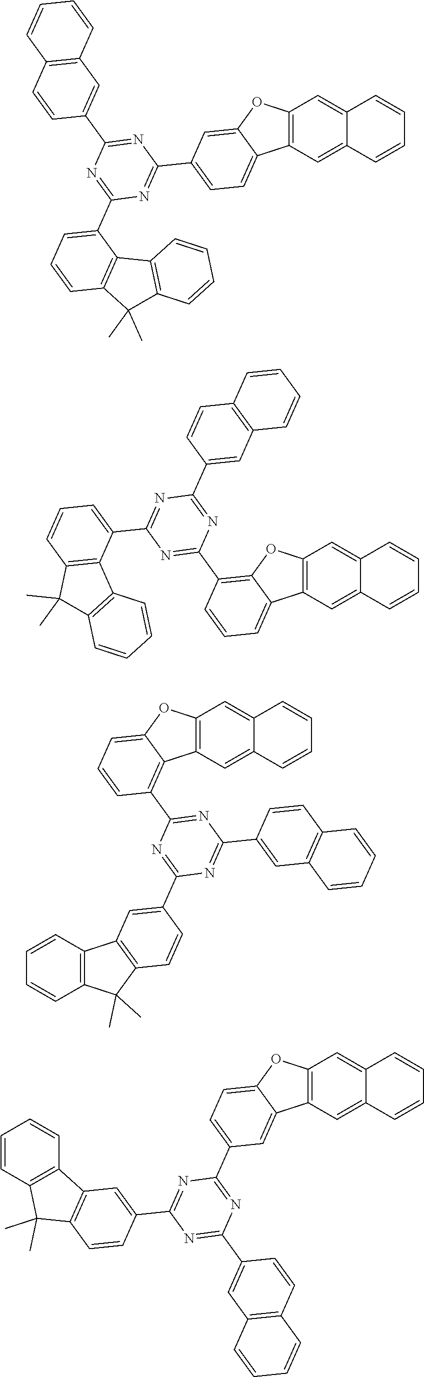 [SMILES: CC1(C)c2ccccc2-c2c(-c3nc(-c4ccc5ccccc5c4)nc(-c4ccc5c(c4)oc4cc6ccccc6cc45)n3)cccc21 ; CC1(C)c2ccccc2-c2c(-c3nc(-c4ccc5ccccc5c4)nc(-c4cccc5c4oc4cc6ccccc6cc45)n3)cccc21 ; CC1(C)c2ccccc2-c2cc(-c3nc(-c4ccc5ccccc5c4)nc(-c4cccc5oc6cc7ccccc7cc6c45)n3)ccc21 ; CC1(C)c2ccccc2-c2cc(-c3nc(-c4ccc5ccccc5c4)nc(-c4ccc5oc6cc7ccccc7cc6c5c4)n3)ccc21]

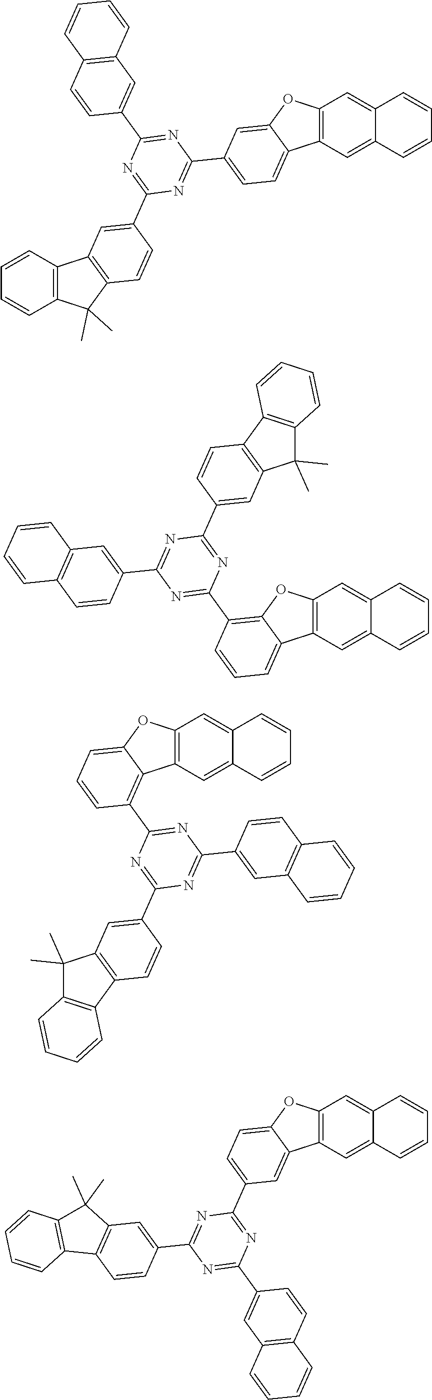

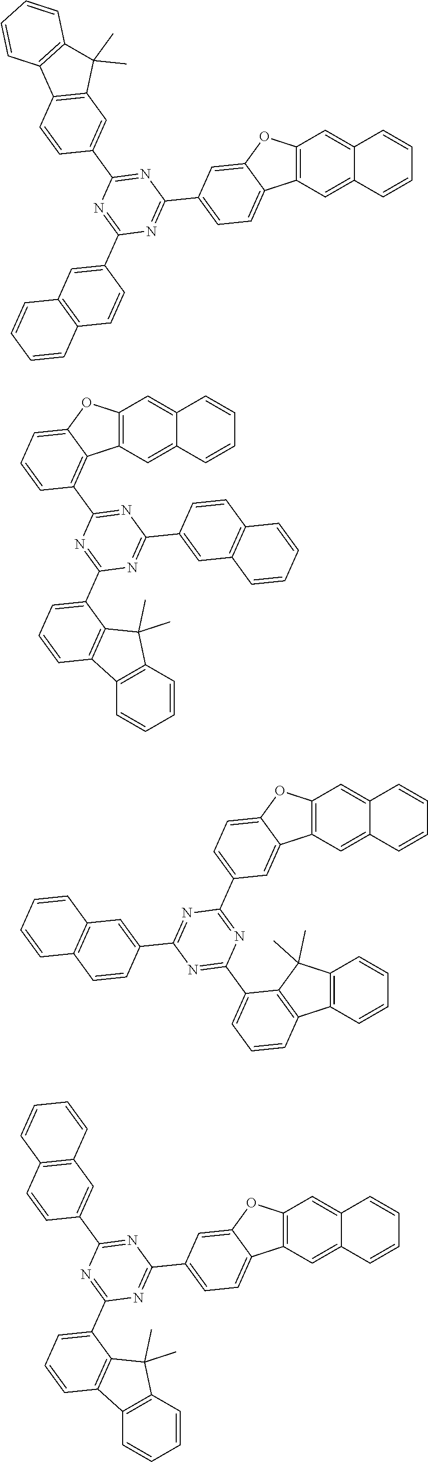 [SMILES: CC1(C)c2ccccc2-c2ccc(-c3nc(-c4ccc5ccccc5c4)nc(-c4ccc5c(c4)oc4cc6ccccc6cc45)n3)cc21 ; CC1(C)c2ccccc2-c2cccc(-c3nc(-c4ccc5ccccc5c4)nc(-c4cccc5oc6cc7ccccc7cc6c45)n3)c21 ; CC1(C)c2ccccc2-c2cccc(-c3nc(-c4ccc5ccccc5c4)nc(-c4ccc5oc6cc7ccccc7cc6c5c4)n3)c21 ; CC1(C)c2ccccc2-c2cccc(-c3nc(-c4ccc5ccccc5c4)nc(-c4ccc5c(c4)oc4cc6ccccc6cc45)n3)c21]

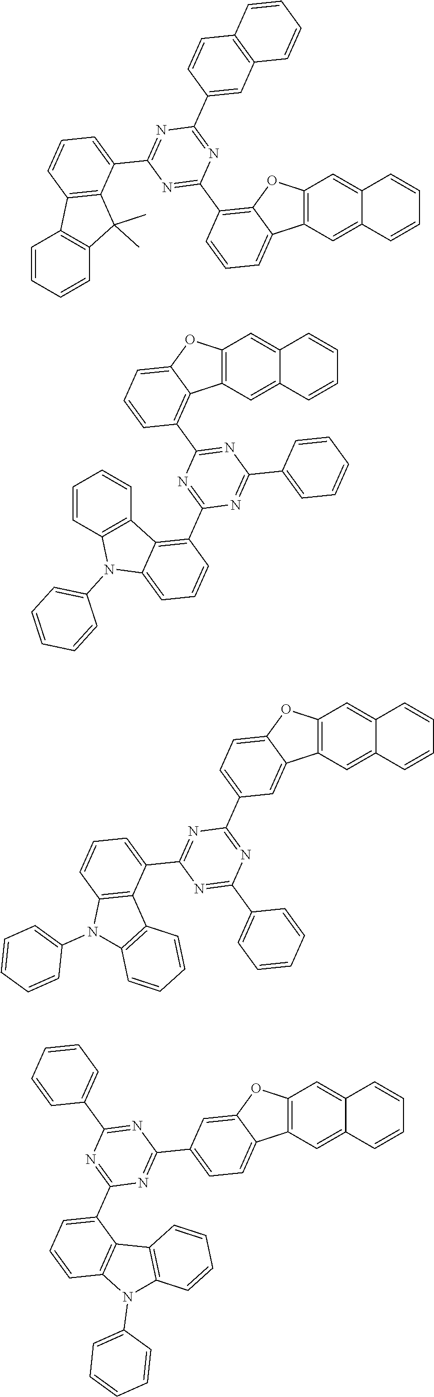 [SMILES: CC1(C)c2ccccc2-c2cccc(-c3nc(-c4ccc5ccccc5c4)nc(-c4cccc5c4oc4cc6ccccc6cc45)n3)c21 ; c1ccc(-c2nc(-c3cccc4oc5cc6ccccc6cc5c34)nc(-c3cccc4c3c3ccccc3n4-c3ccccc3)n2)cc1 ; c1ccc(-c2nc(-c3ccc4oc5cc6ccccc6cc5c4c3)nc(-c3cccc4c3c3ccccc3n4-c3ccccc3)n2)cc1 ; c1ccc(-c2nc(-c3ccc4c(c3)oc3cc5ccccc5cc34)nc(-c3cccc4c3c3ccccc3n4-c3ccccc3)n2)cc1]

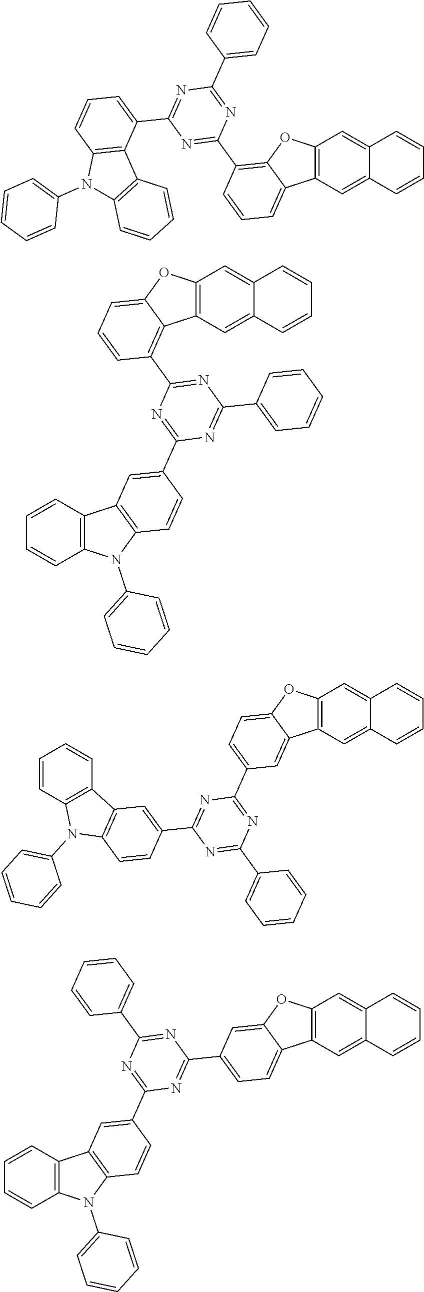

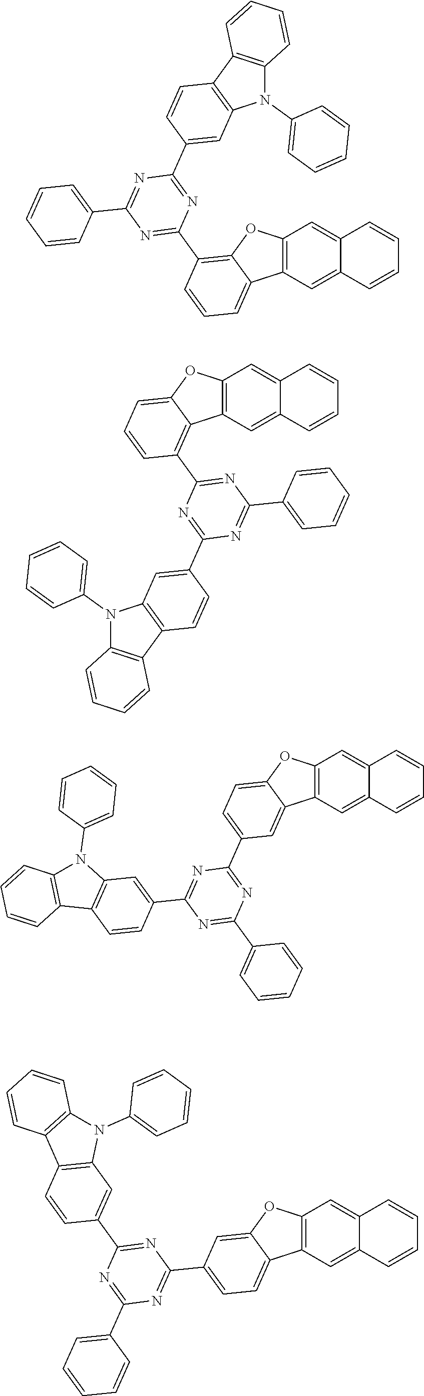 [SMILES: c1ccc(-c2nc(-c3ccc4c5ccccc5n(-c5ccccc5)c4c3)nc(-c3cccc4c3oc3cc5ccccc5cc34)n2)cc1 ; c1ccc(-c2nc(-c3ccc4c5ccccc5n(-c5ccccc5)c4c3)nc(-c3cccc4oc5cc6ccccc6cc5c34)n2)cc1 ; c1ccc(-c2nc(-c3ccc4oc5cc6ccccc6cc5c4c3)nc(-c3ccc4c5ccccc5n(-c5ccccc5)c4c3)n2)cc1 ; c1ccc(-c2nc(-c3ccc4c(c3)oc3cc5ccccc5cc34)nc(-c3ccc4c5ccccc5n(-c5ccccc5)c4c3)n2)cc1]

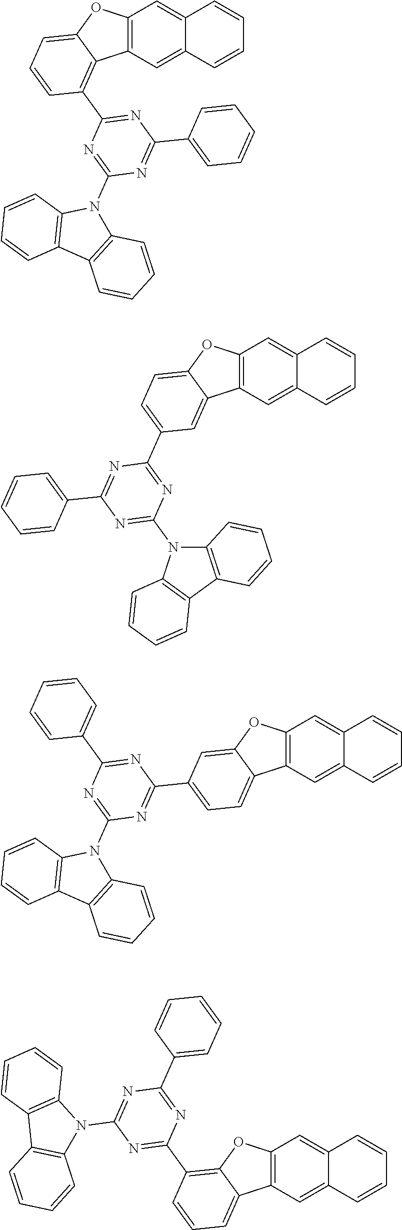 [SMILES: c1ccc(-c2nc(-c3cccc4oc5cc6ccccc6cc5c34)nc(-n3c4ccccc4c4ccccc43)n2)cc1 ; c1ccc(-c2nc(-c3ccc4oc5cc6ccccc6cc5c4c3)nc(-n3c4ccccc4c4ccccc43)n2)cc1 ; c1ccc(-c2nc(-c3ccc4c(c3)oc3cc5ccccc5cc34)nc(-n3c4ccccc4c4ccccc43)n2)cc1 ; c1ccc(-c2nc(-c3cccc4c3oc3cc5ccccc5cc34)nc(-n3c4ccccc4c4ccccc43)n2)cc1]

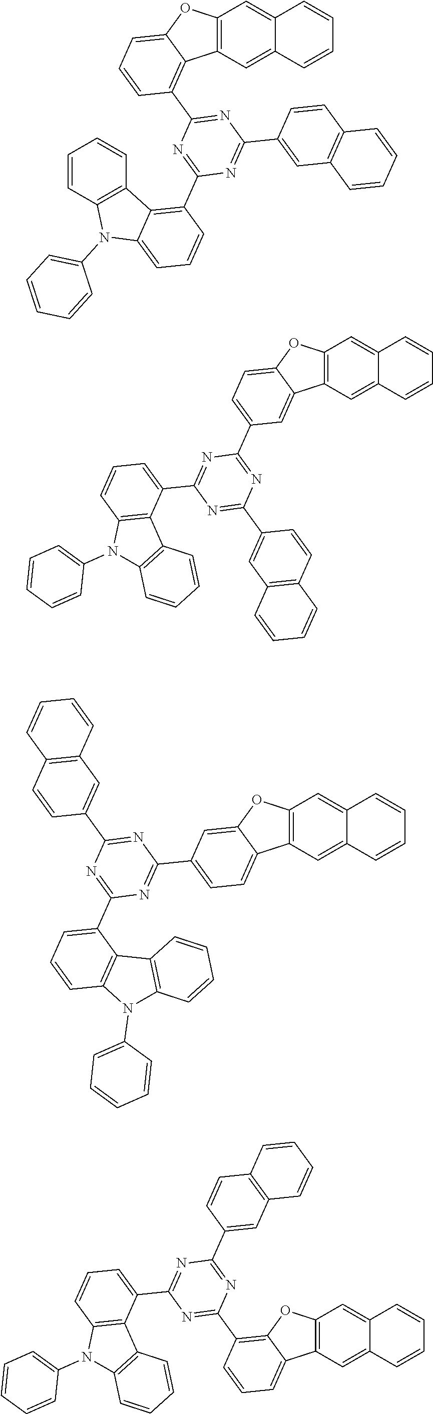 [SMILES: c1ccc(-n2c3ccccc3c3c(-c4nc(-c5ccc6ccccc6c5)nc(-c5cccc6oc7cc8ccccc8cc7c56)n4)cccc32)cc1 ; c1ccc(-n2c3ccccc3c3c(-c4nc(-c5ccc6ccccc6c5)nc(-c5ccc6oc7cc8ccccc8cc7c6c5)n4)cccc32)cc1 ; c1ccc(-n2c3ccccc3c3c(-c4nc(-c5ccc6ccccc6c5)nc(-c5ccc6c(c5)oc5cc7ccccc7cc56)n4)cccc32)cc1 ; c1ccc(-n2c3ccccc3c3c(-c4nc(-c5ccc6ccccc6c5)nc(-c5cccc6c5oc5cc7ccccc7cc56)n4)cccc32)cc1]

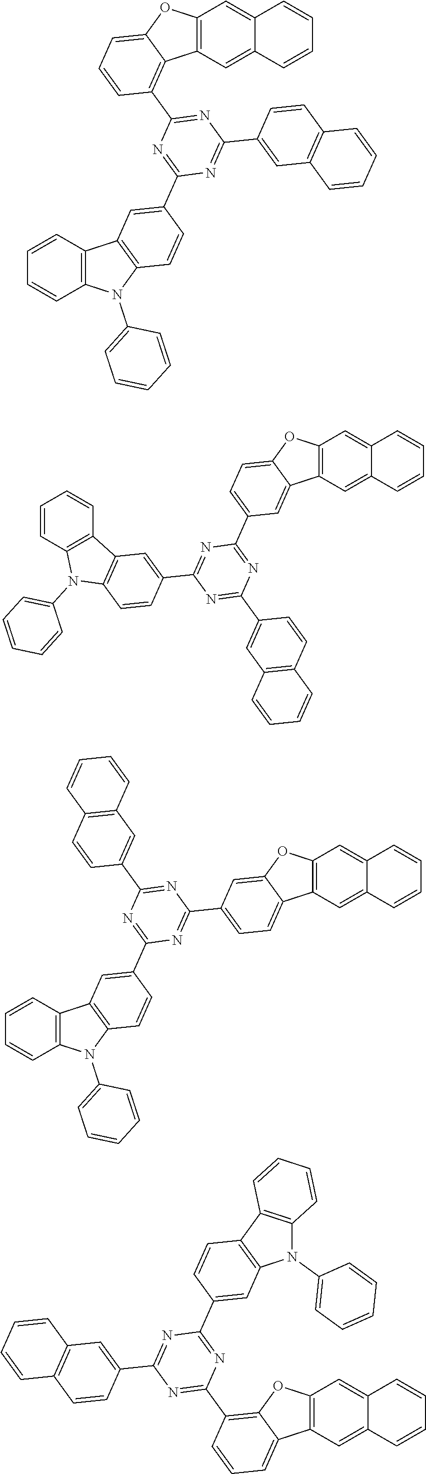 [SMILES: c1ccc(-n2c3ccccc3c3cc(-c4nc(-c5ccc6ccccc6c5)nc(-c5cccc6oc7cc8ccccc8cc7c56)n4)ccc32)cc1 ; c1ccc(-n2c3ccccc3c3cc(-c4nc(-c5ccc6ccccc6c5)nc(-c5ccc6oc7cc8ccccc8cc7c6c5)n4)ccc32)cc1 ; c1ccc(-n2c3ccccc3c3cc(-c4nc(-c5ccc6ccccc6c5)nc(-c5ccc6c(c5)oc5cc7ccccc7cc56)n4)ccc32)cc1 ; c1ccc(-n2c3ccccc3c3ccc(-c4nc(-c5ccc6ccccc6c5)nc(-c5cccc6c5oc5cc7ccccc7cc56)n4)cc32)cc1]

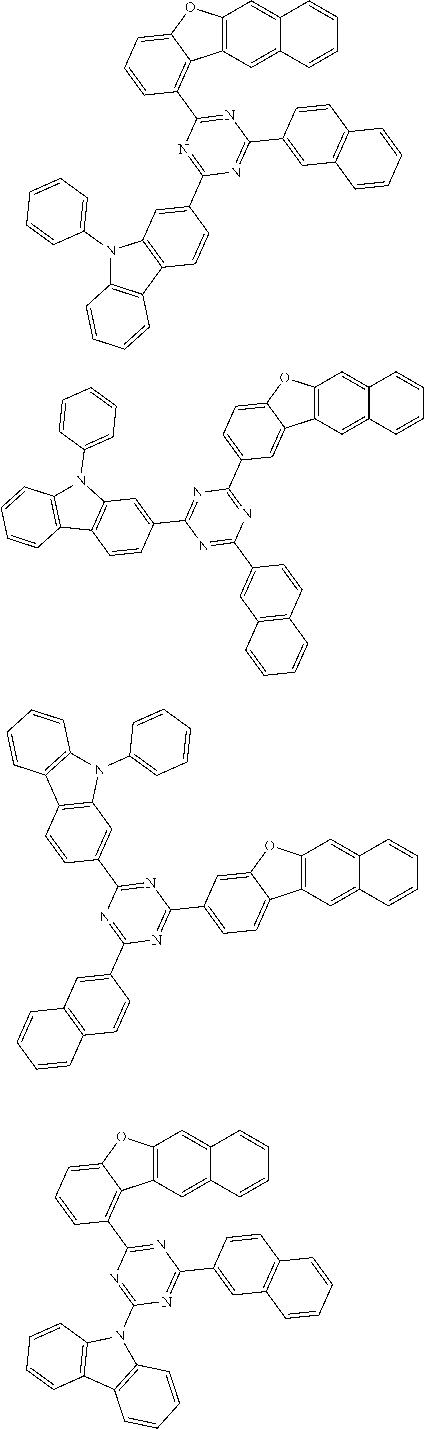 [SMILES: c1ccc(-n2c3ccccc3c3ccc(-c4nc(-c5ccc6ccccc6c5)nc(-c5cccc6oc7cc8ccccc8cc7c56)n4)cc32)cc1 ; c1ccc(-n2c3ccccc3c3ccc(-c4nc(-c5ccc6ccccc6c5)nc(-c5ccc6oc7cc8ccccc8cc7c6c5)n4)cc32)cc1 ; c1ccc(-n2c3ccccc3c3ccc(-c4nc(-c5ccc6ccccc6c5)nc(-c5ccc6c(c5)oc5cc7ccccc7cc56)n4)cc32)cc1 ; c1ccc2cc(-c3nc(-c4cccc5oc6cc7ccccc7cc6c45)nc(-n4c5ccccc5c5ccccc54)n3)ccc2c1]

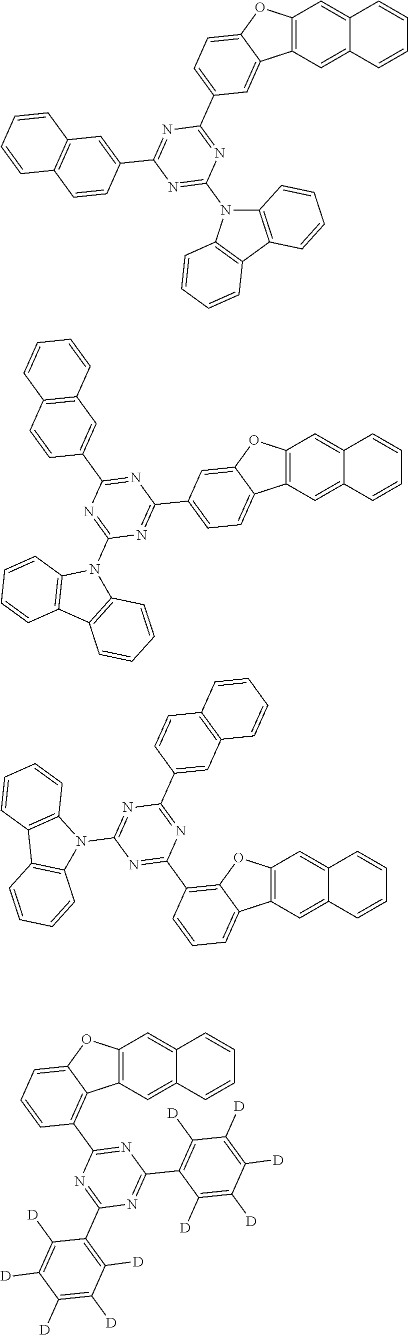 [SMILES: c1ccc2cc(-c3nc(-c4ccc5oc6cc7ccccc7cc6c5c4)nc(-n4c5ccccc5c5ccccc54)n3)ccc2c1 ; c1ccc2cc(-c3nc(-c4ccc5c(c4)oc4cc6ccccc6cc45)nc(-n4c5ccccc5c5ccccc54)n3)ccc2c1 ; c1ccc2cc(-c3nc(-c4cccc5c4oc4cc6ccccc6cc45)nc(-n4c5ccccc5c5ccccc54)n3)ccc2c1 ; [2H]c1c([2H])c([2H])c(-c2nc(-c3c([2H])c([2H])c([2H])c([2H])c3[2H])nc(-c3cccc4oc5cc6ccccc6cc5c34)n2)c([2H])c1[2H]]

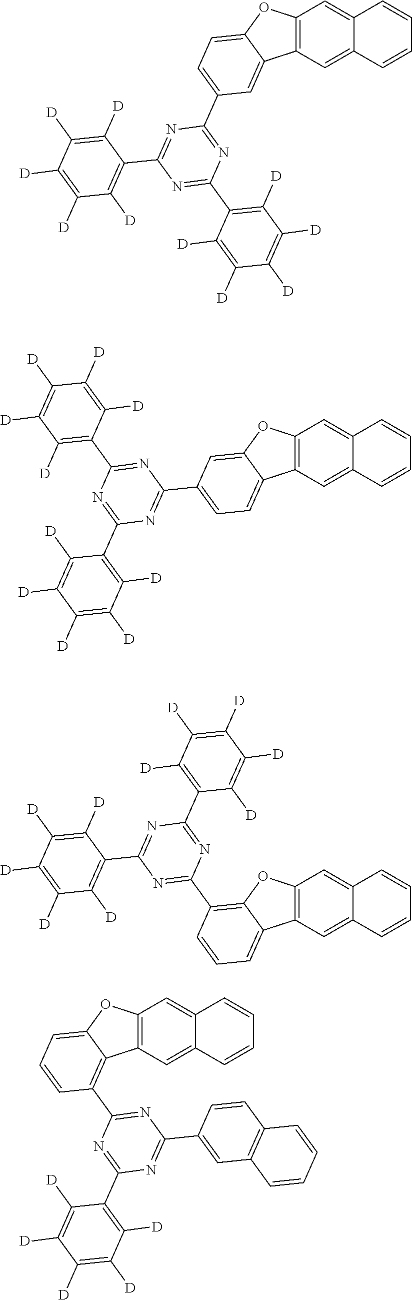

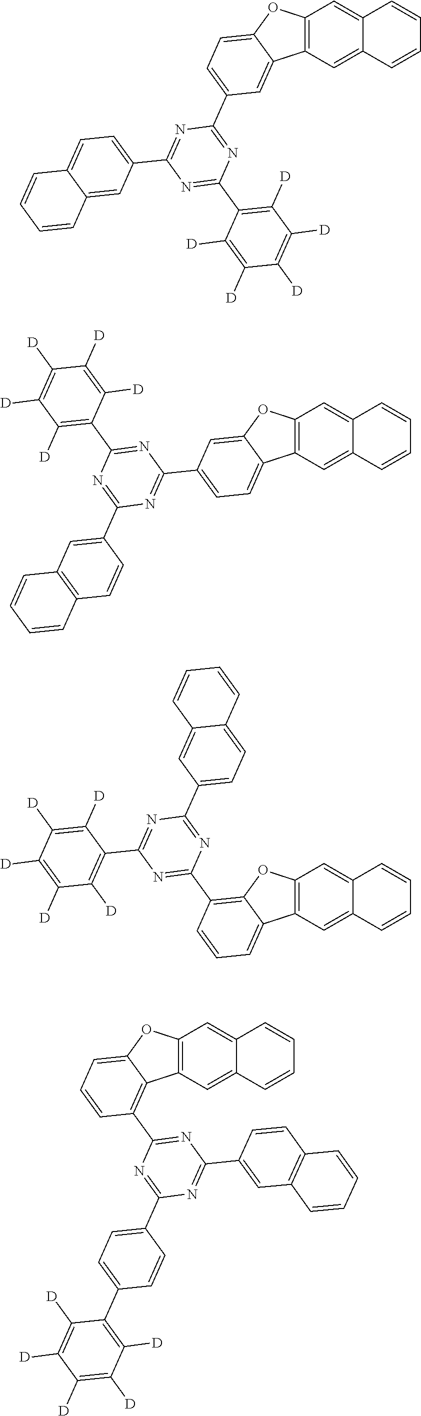 [SMILES: [2H]c1c([2H])c([2H])c(-c2nc(-c3ccc4ccccc4c3)nc(-c3ccc4oc5cc6ccccc6cc5c4c3)n2)c([2H])c1[2H] ; [2H]c1c([2H])c([2H])c(-c2nc(-c3ccc4ccccc4c3)nc(-c3ccc4c(c3)oc3cc5ccccc5cc34)n2)c([2H])c1[2H] ; [2H]c1c([2H])c([2H])c(-c2nc(-c3ccc4ccccc4c3)nc(-c3cccc4c3oc3cc5ccccc5cc34)n2)c([2H])c1[2H] ; [2H]c1c([2H])c([2H])c(-c2ccc(-c3nc(-c4ccc5ccccc5c4)nc(-c4cccc5oc6cc7ccccc7cc6c45)n3)cc2)c([2H])c1[2H]]

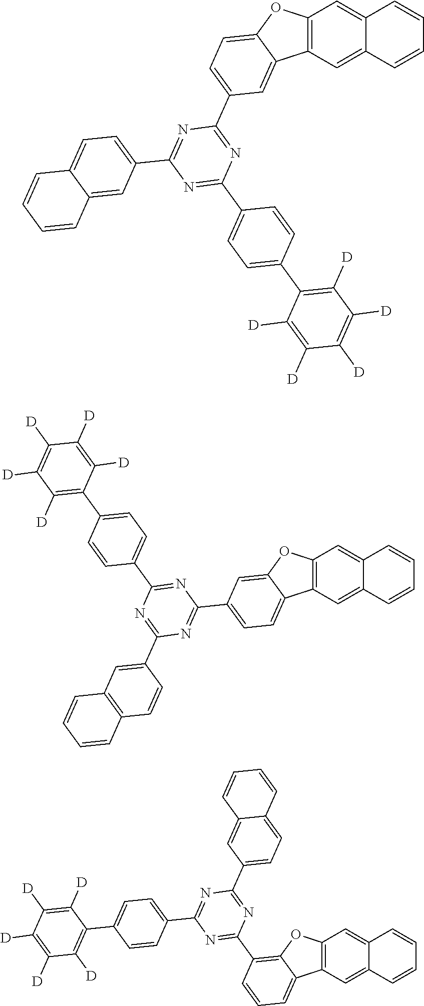 [SMILES: [2H]c1c([2H])c([2H])c(-c2ccc(-c3nc(-c4ccc5ccccc5c4)nc(-c4ccc5oc6cc7ccccc7cc6c5c4)n3)cc2)c([2H])c1[2H] ; [2H]c1c([2H])c([2H])c(-c2ccc(-c3nc(-c4ccc5ccccc5c4)nc(-c4ccc5c(c4)oc4cc6ccccc6cc45)n3)cc2)c([2H])c1[2H] ; [2H]c1c([2H])c([2H])c(-c2ccc(-c3nc(-c4ccc5ccccc5c4)nc(-c4cccc5c4oc4cc6ccccc6cc45)n3)cc2)c([2H])c1[2H]]

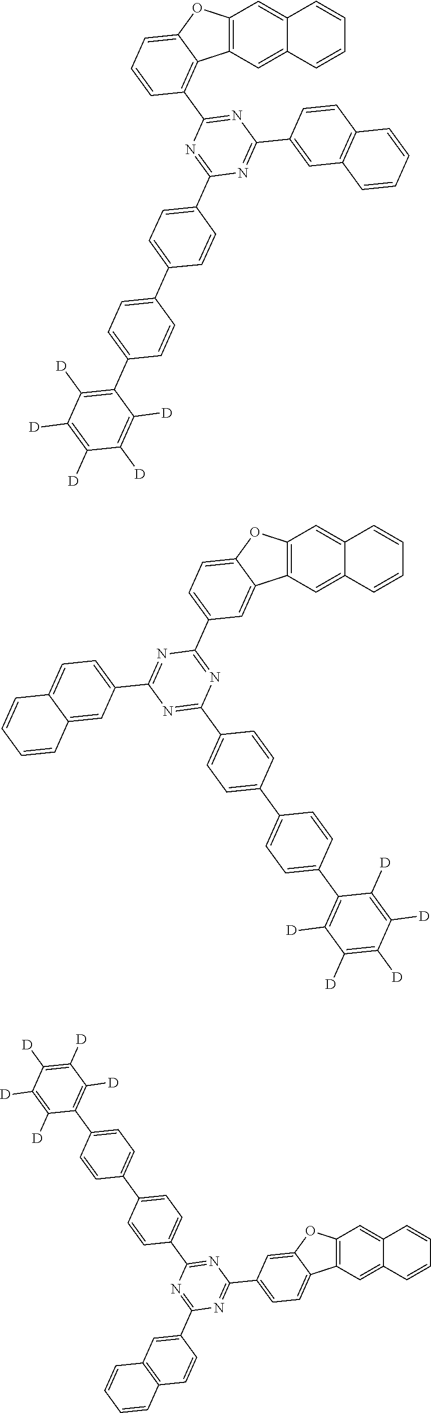 [SMILES: [2H]c1c([2H])c([2H])c(-c2ccc(-c3ccc(-c4nc(-c5ccc6ccccc6c5)nc(-c5cccc6oc7cc8ccccc8cc7c56)n4)cc3)cc2)c([2H])c1[2H] ; [2H]c1c([2H])c([2H])c(-c2ccc(-c3ccc(-c4nc(-c5ccc6ccccc6c5)nc(-c5ccc6oc7cc8ccccc8cc7c6c5)n4)cc3)cc2)c([2H])c1[2H] ; [2H]c1c([2H])c([2H])c(-c2ccc(-c3ccc(-c4nc(-c5ccc6ccccc6c5)nc(-c5ccc6c(c5)oc5cc7ccccc7cc56)n4)cc3)cc2)c([2H])c1[2H]]

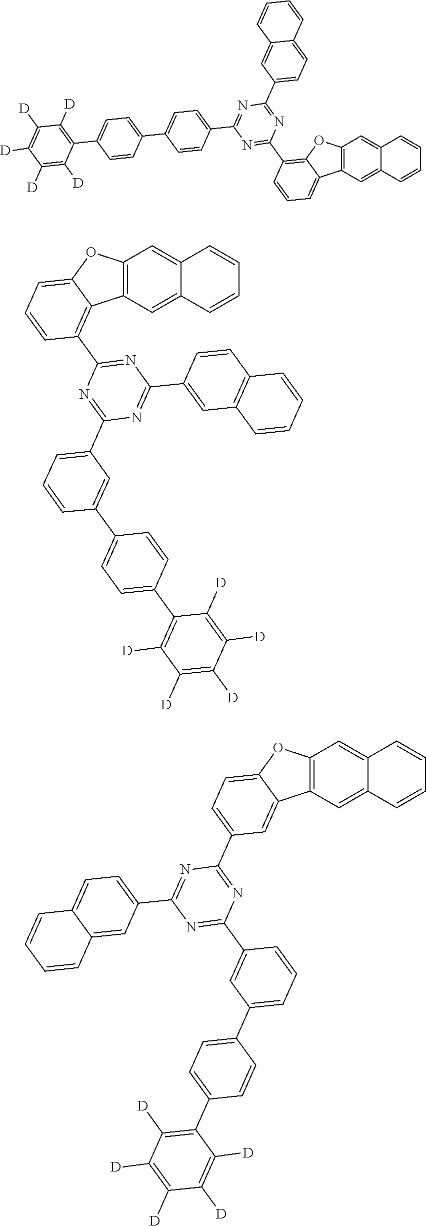 [SMILES: [2H]c1c([2H])c([2H])c(-c2ccc(-c3ccc(-c4nc(-c5ccc6ccccc6c5)nc(-c5cccc6c5oc5cc7ccccc7cc56)n4)cc3)cc2)c([2H])c1[2H] ; [2H]c1c([2H])c([2H])c(-c2ccc(-c3cccc(-c4nc(-c5ccc6ccccc6c5)nc(-c5cccc6oc7cc8ccccc8cc7c56)n4)c3)cc2)c([2H])c1[2H] ; [2H]c1c([2H])c([2H])c(-c2ccc(-c3cccc(-c4nc(-c5ccc6ccccc6c5)nc(-c5ccc6oc7cc8ccccc8cc7c6c5)n4)c3)cc2)c([2H])c1[2H]]

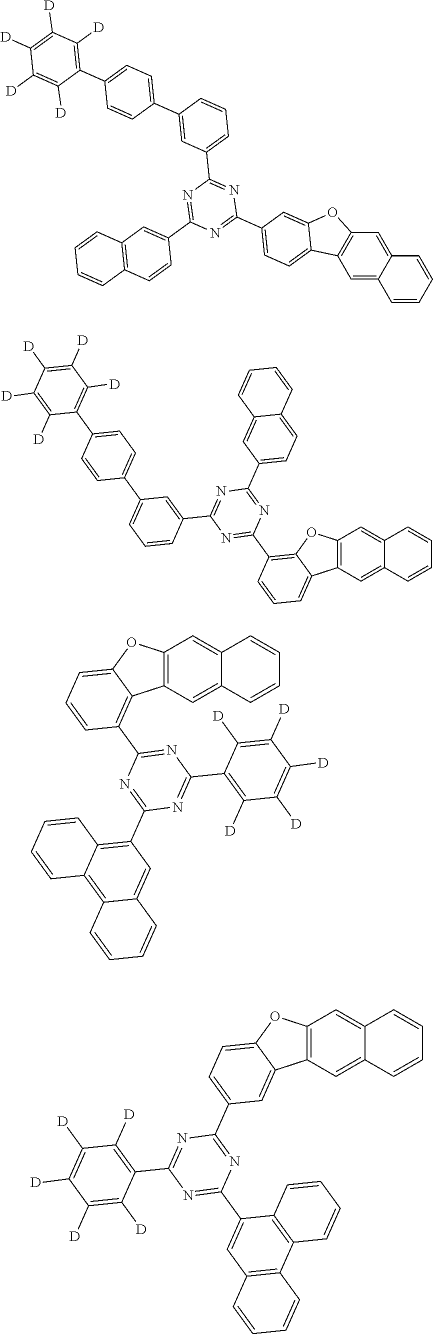 [SMILES: [2H]c1c([2H])c([2H])c(-c2ccc(-c3cccc(-c4nc(-c5ccc6ccccc6c5)nc(-c5ccc6c(c5)oc5cc7ccccc7cc56)n4)c3)cc2)c([2H])c1[2H] ; [2H]c1c([2H])c([2H])c(-c2ccc(-c3cccc(-c4nc(-c5ccc6ccccc6c5)nc(-c5cccc6c5oc5cc7ccccc7cc56)n4)c3)cc2)c([2H])c1[2H] ; [2H]c1c([2H])c([2H])c(-c2nc(-c3cc4ccccc4c4ccccc34)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cc4ccccc4c4ccccc34)n2)c([2H])c1[2H]]

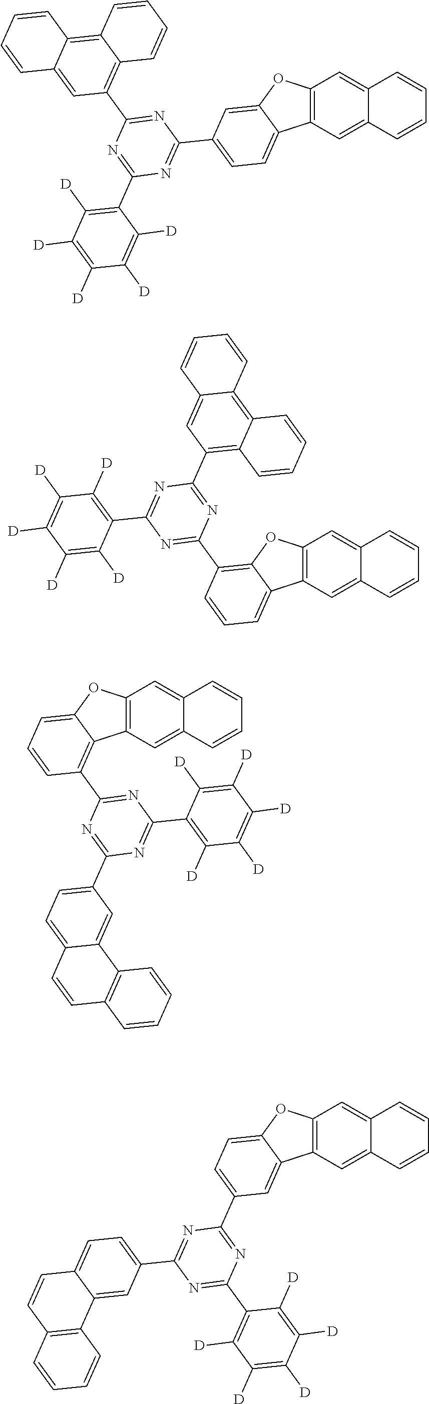 [SMILES: [2H]c1c([2H])c([2H])c(-c2nc(-c3ccc4c(c3)oc3cc5ccccc5cc34)nc(-c3cc4ccccc4c4ccccc34)n2)c([2H])c1[2H] ; [2H]c1c([2H])c([2H])c(-c2nc(-c3cc4ccccc4c4ccccc34)nc(-c3cccc4c3oc3cc5ccccc5cc34)n2)c([2H])c1[2H] ; [2H]c1c([2H])c([2H])c(-c2nc(-c3ccc4ccc5ccccc5c4c3)nc(-c3cccc4oc5cc6ccccc6cc5c34)n2)c([2H])c1[2H] ; [2H]c1c([2H])c([2H])c(-c2nc(-c3ccc4ccc5ccccc5c4c3)nc(-c3ccc4oc5cc6ccccc6cc5c4c3)n2)c([2H])c1[2H]]

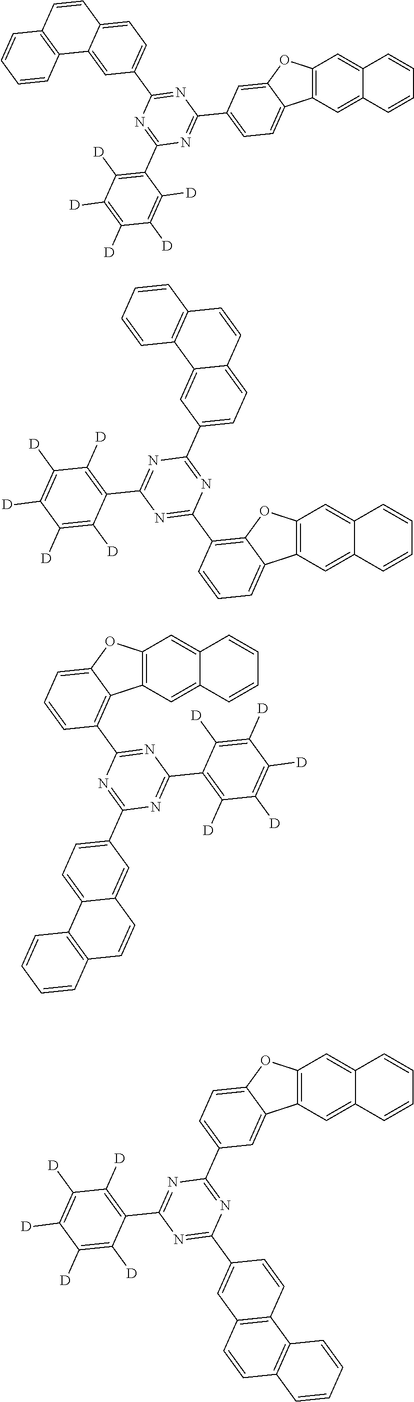

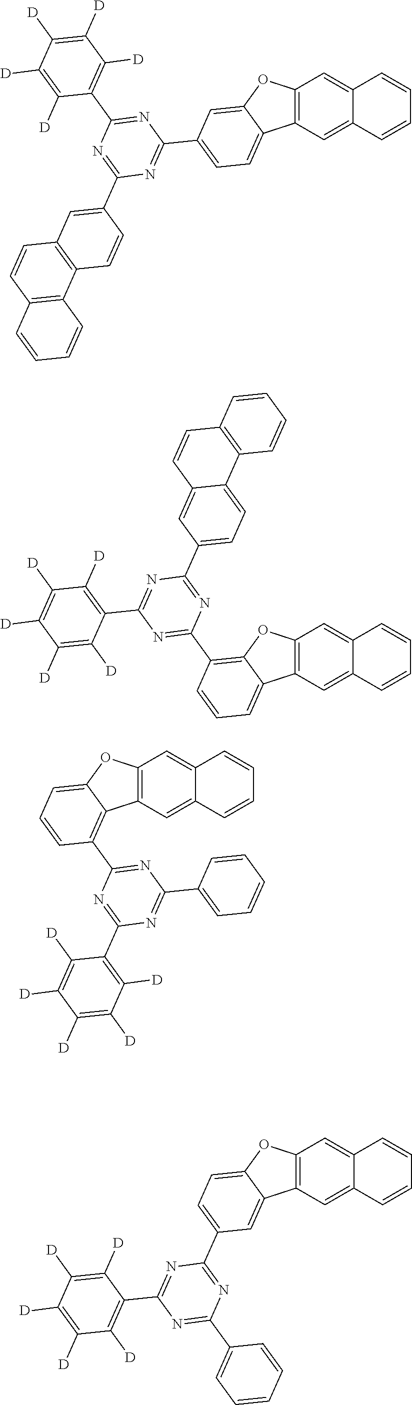

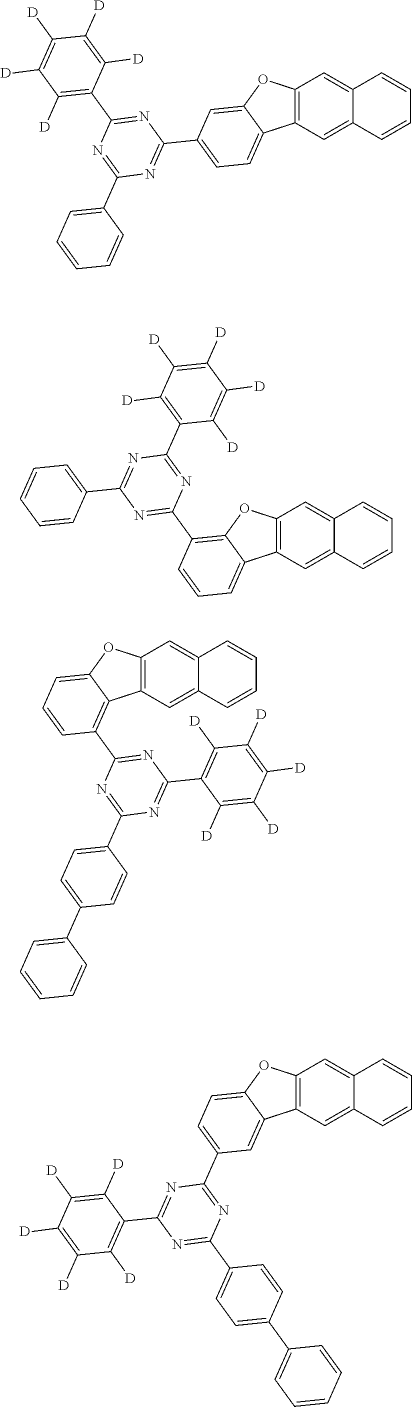 [SMILES: [2H]c1c([2H])c([2H])c(-c2nc(-c3ccccc3)nc(-c3ccc4c(c3)oc3cc5ccccc5cc34)n2)c([2H])c1[2H] ; [2H]c1c([2H])c([2H])c(-c2nc(-c3ccccc3)nc(-c3cccc4c3oc3cc5ccccc5cc34)n2)c([2H])c1[2H] ; [2H]c1c([2H])c([2H])c(-c2nc(-c3ccc(-c4ccccc4)cc3)nc(-c3cccc4oc5cc6ccccc6cc5c34)n2)c([2H])c1[2H] ; [2H]c1c([2H])c([2H])c(-c2nc(-c3ccc(-c4ccccc4)cc3)nc(-c3ccc4oc5cc6ccccc6cc5c4c3)n2)c([2H])c1[2H]]

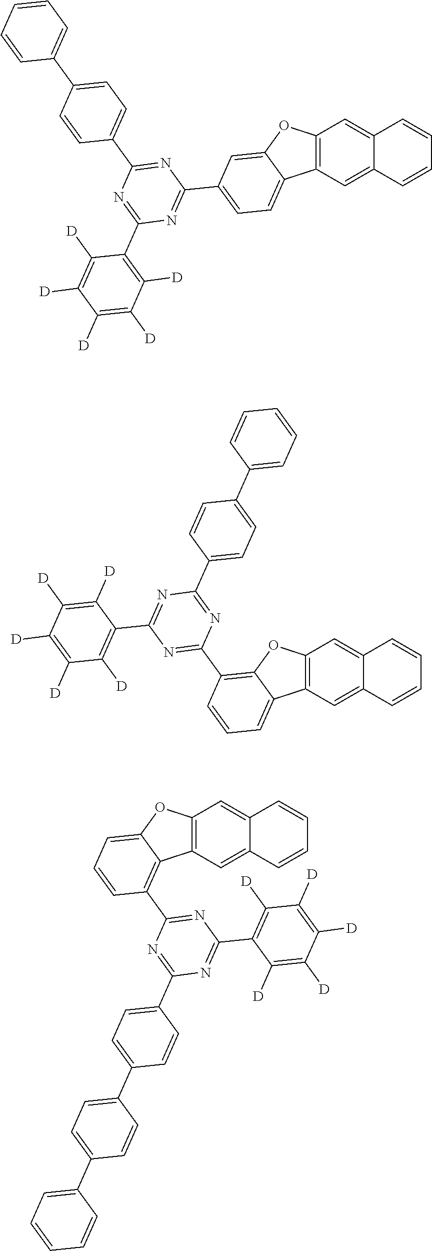 [SMILES: [2H]c1c([2H])c([2H])c(-c2nc(-c3ccc(-c4ccccc4)cc3)nc(-c3ccc4c(c3)oc3cc5ccccc5cc34)n2)c([2H])c1[2H] ; [2H]c1c([2H])c([2H])c(-c2nc(-c3ccc(-c4ccccc4)cc3)nc(-c3cccc4c3oc3cc5ccccc5cc34)n2)c([2H])c1[2H] ; [2H]c1c([2H])c([2H])c(-c2nc(-c3ccc(-c4ccc(-c5ccccc5)cc4)cc3)nc(-c3cccc4oc5cc6ccccc6cc5c34)n2)c([2H])c1[2H]]

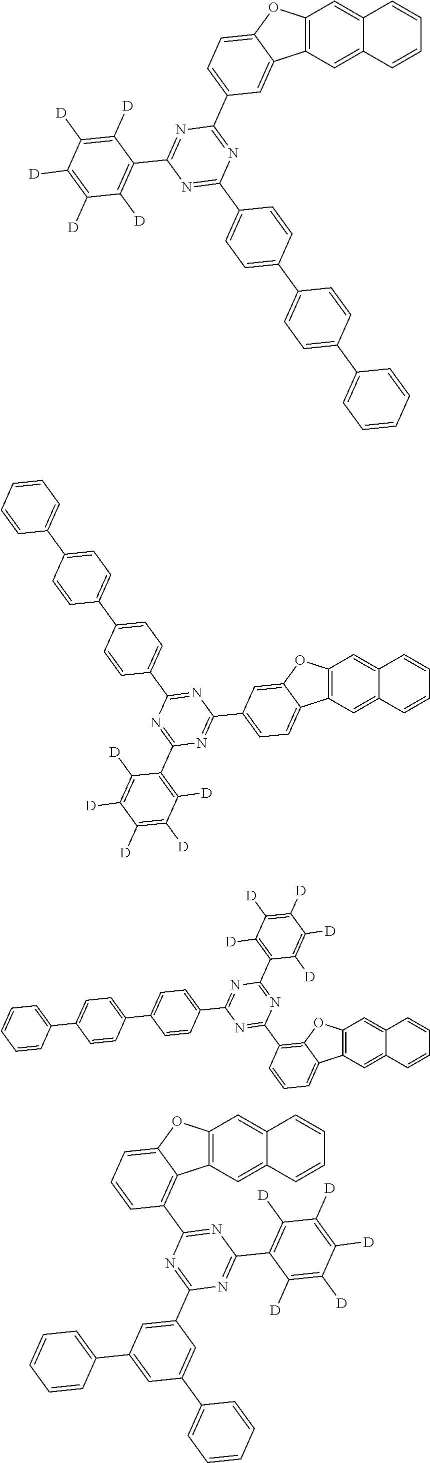

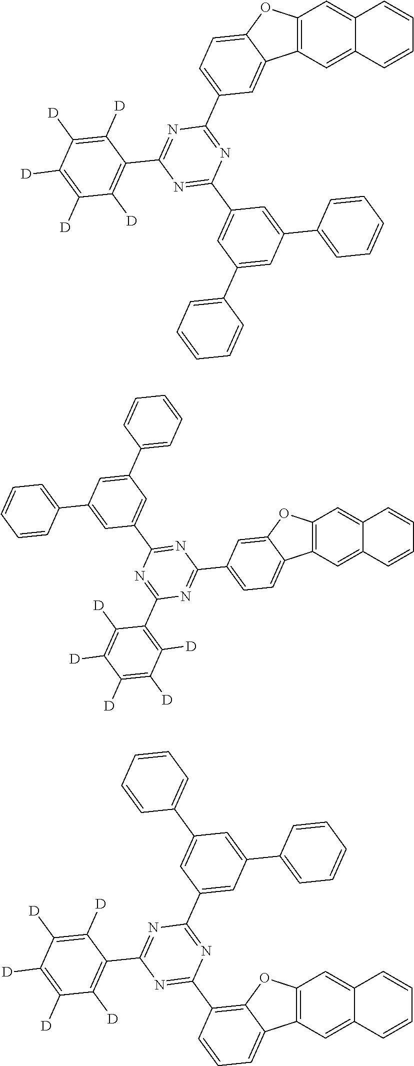 [SMILES: [2H]c1c([2H])c([2H])c(-c2nc(-c3cc(-c4ccccc4)cc(-c4ccccc4)c3)nc(-c3ccc4oc5cc6ccccc6cc5c4c3)n2)c([2H])c1[2H] ; [2H]c1c([2H])c([2H])c(-c2nc(-c3cc(-c4ccccc4)cc(-c4ccccc4)c3)nc(-c3ccc4c(c3)oc3cc5ccccc5cc34)n2)c([2H])c1[2H] ; [2H]c1c([2H])c([2H])c(-c2nc(-c3cc(-c4ccccc4)cc(-c4ccccc4)c3)nc(-c3cccc4c3oc3cc5ccccc5cc34)n2)c([2H])c1[2H]]

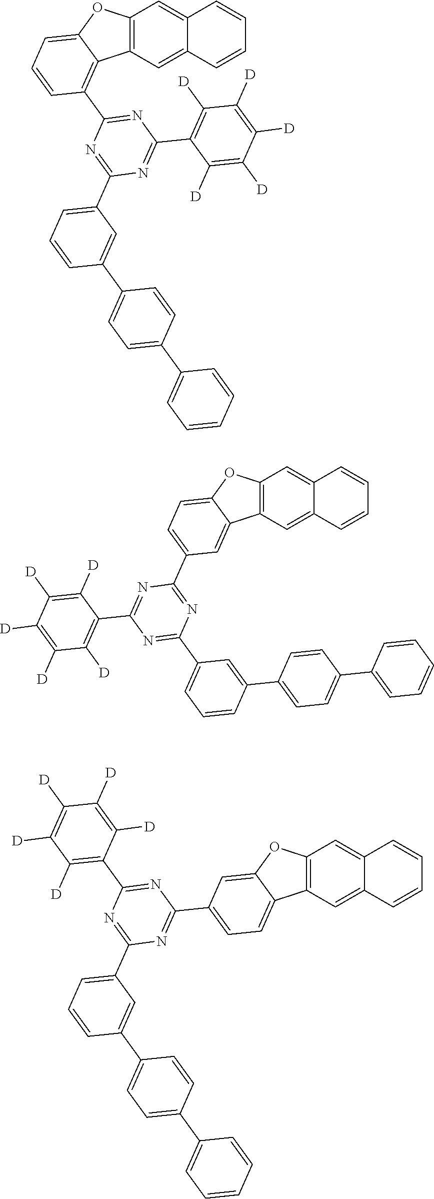 [SMILES: [2H]c1c([2H])c([2H])c(-c2nc(-c3cccc(-c4ccc(-c5ccccc5)cc4)c3)nc(-c3cccc4oc5cc6ccccc6cc5c34)n2)c([2H])c1[2H] ; [2H]c1c([2H])c([2H])c(-c2nc(-c3cccc(-c4ccc(-c5ccccc5)cc4)c3)nc(-c3ccc4oc5cc6ccccc6cc5c4c3)n2)c([2H])c1[2H] ; [2H]c1c([2H])c([2H])c(-c2nc(-c3cccc(-c4ccc(-c5ccccc5)cc4)c3)nc(-c3ccc4c(c3)oc3cc5ccccc5cc34)n2)c([2H])c1[2H]]

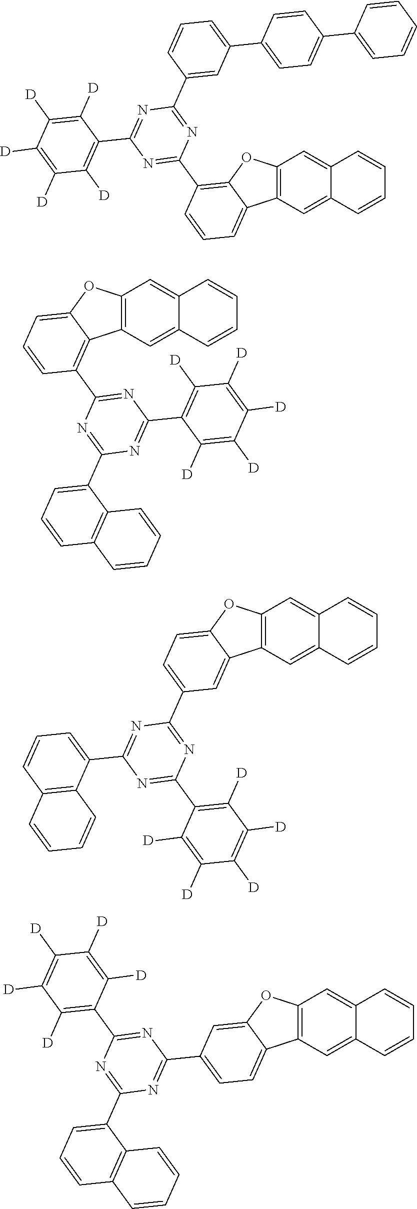

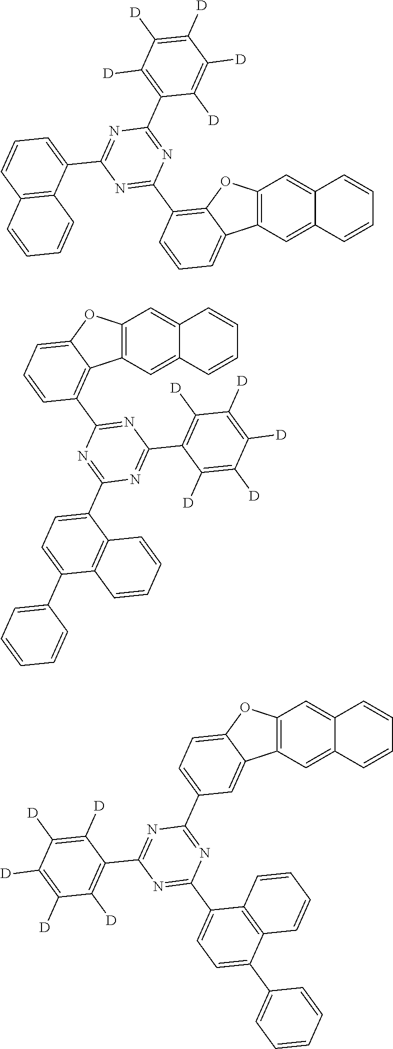

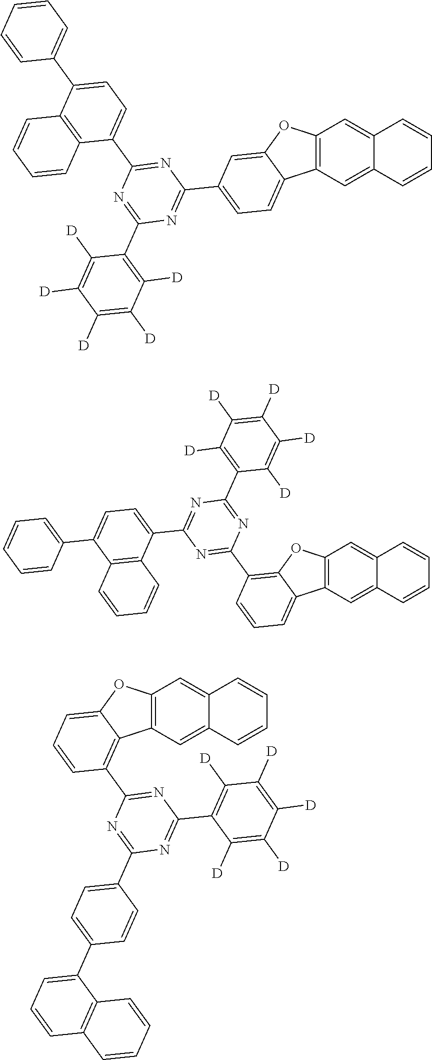 [SMILES: [2H]c1c([2H])c([2H])c(-c2nc(-c3ccc4c(c3)oc3cc5ccccc5cc34)nc(-c3ccc(-c4ccccc4)c4ccccc34)n2)c([2H])c1[2H] ; [2H]c1c([2H])c([2H])c(-c2nc(-c3ccc(-c4ccccc4)c4ccccc34)nc(-c3cccc4c3oc3cc5ccccc5cc34)n2)c([2H])c1[2H] ; [2H]c1c([2H])c([2H])c(-c2nc(-c3ccc(-c4cccc5ccccc45)cc3)nc(-c3cccc4oc5cc6ccccc6cc5c34)n2)c([2H])c1[2H]]

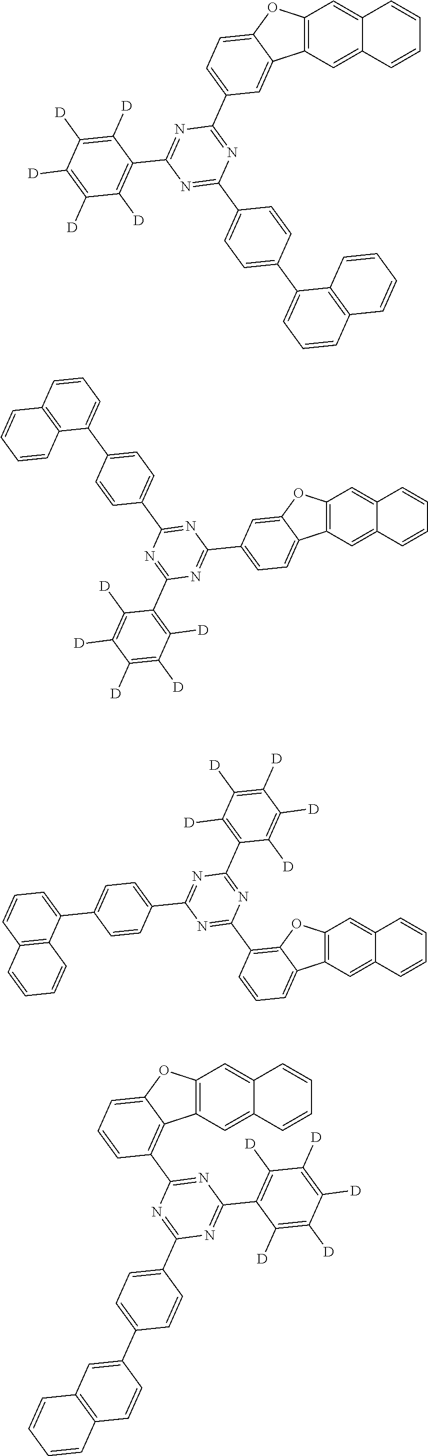 [SMILES: [2H]c1c([2H])c([2H])c(-c2nc(-c3ccc(-c4cccc5ccccc45)cc3)nc(-c3ccc4oc5cc6ccccc6cc5c4c3)n2)c([2H])c1[2H] ; [2H]c1c([2H])c([2H])c(-c2nc(-c3ccc(-c4cccc5ccccc45)cc3)nc(-c3ccc4c(c3)oc3cc5ccccc5cc34)n2)c([2H])c1[2H] ; [2H]c1c([2H])c([2H])c(-c2nc(-c3ccc(-c4cccc5ccccc45)cc3)nc(-c3cccc4c3oc3cc5ccccc5cc34)n2)c([2H])c1[2H] ; [2H]c1c([2H])c([2H])c(-c2nc(-c3ccc(-c4ccc5ccccc5c4)cc3)nc(-c3cccc4oc5cc6ccccc6cc5c34)n2)c([2H])c1[2H]]

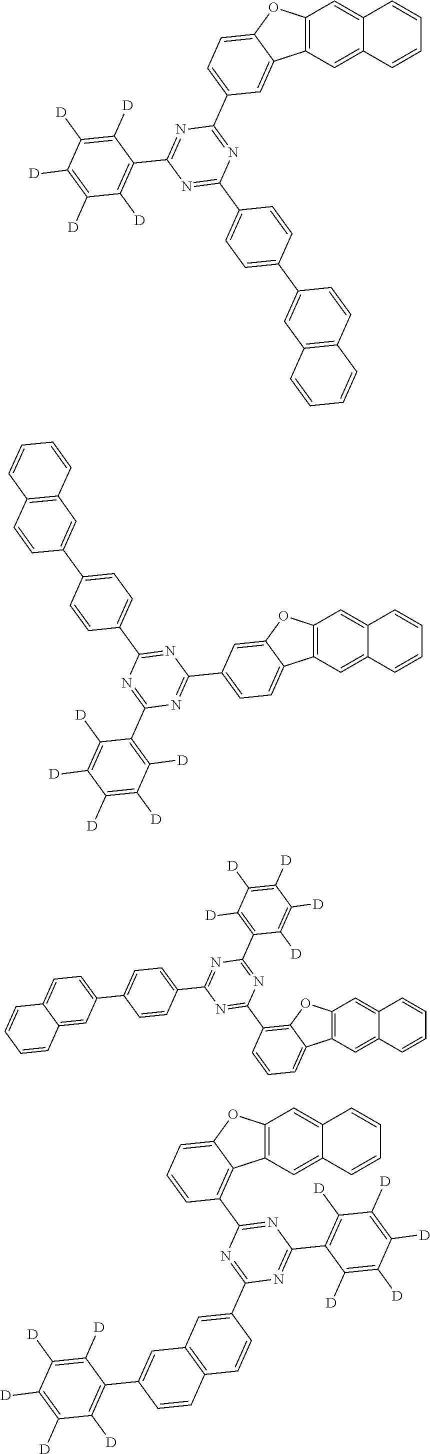

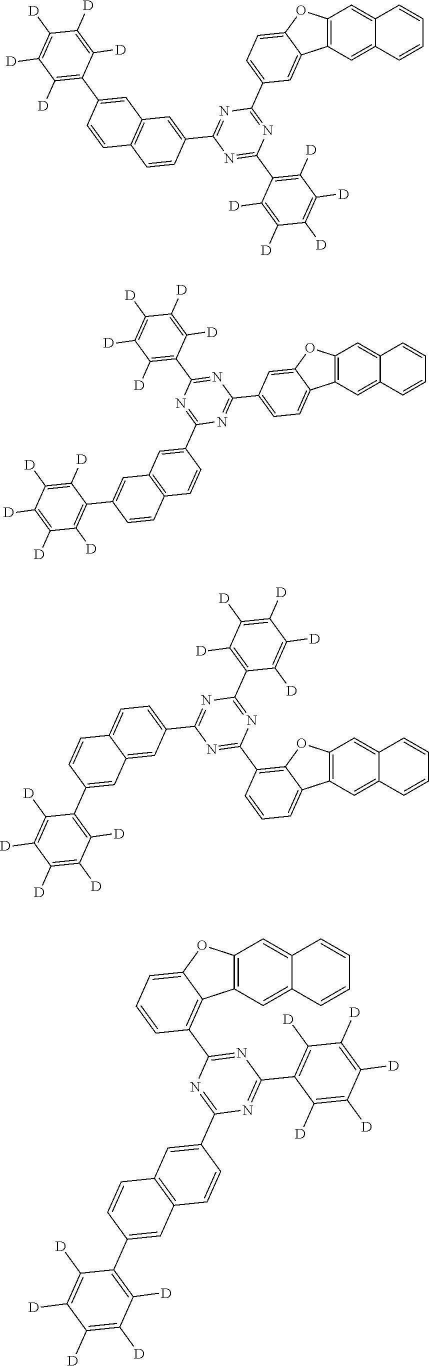

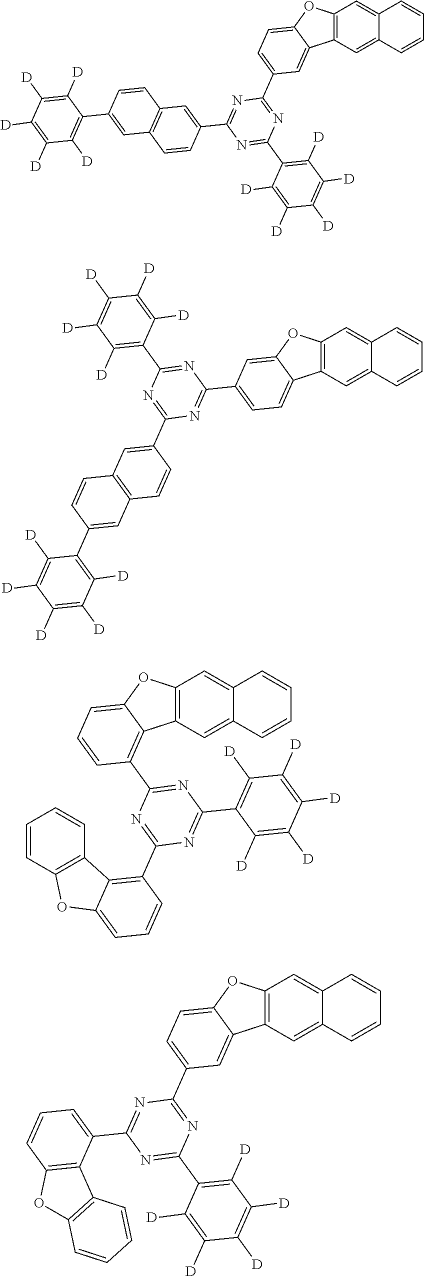 [SMILES: [2H]c1c([2H])c([2H])c(-c2ccc3cc(-c4nc(-c5ccc6oc7cc8ccccc8cc7c6c5)nc(-c5c([2H])c([2H])c([2H])c([2H])c5[2H])n4)ccc3c2)c([2H])c1[2H] ; [2H]c1c([2H])c([2H])c(-c2ccc3cc(-c4nc(-c5ccc6c(c5)oc5cc7ccccc7cc56)nc(-c5c([2H])c([2H])c([2H])c([2H])c5[2H])n4)ccc3c2)c([2H])c1[2H] ; [2H]c1c([2H])c([2H])c(-c2nc(-c3cccc4oc5ccccc5c34)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cccc4oc5ccccc5c34)n2)c([2H])c1[2H]]

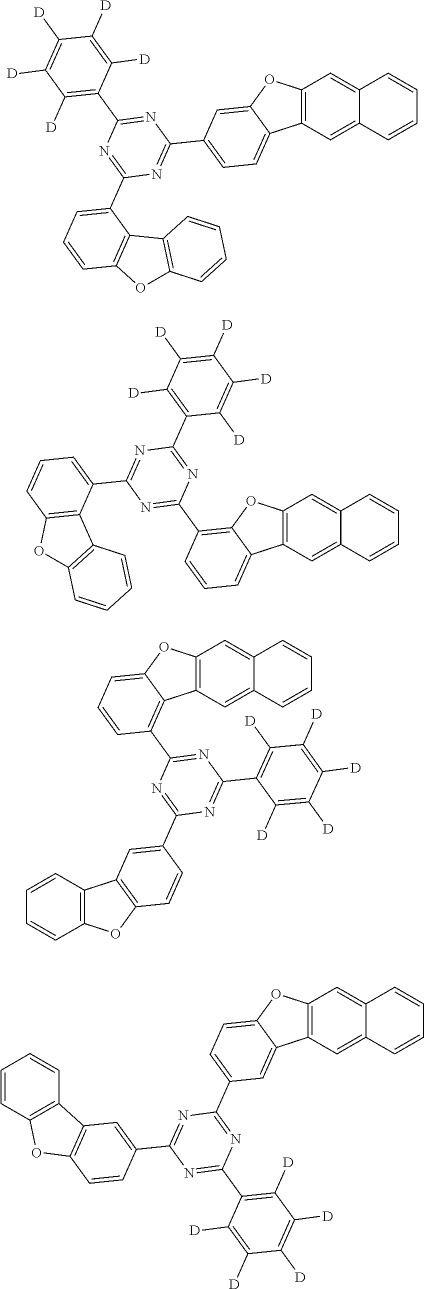 [SMILES: [2H]c1c([2H])c([2H])c(-c2nc(-c3ccc4c(c3)oc3cc5ccccc5cc34)nc(-c3cccc4oc5ccccc5c34)n2)c([2H])c1[2H] ; [2H]c1c([2H])c([2H])c(-c2nc(-c3cccc4c3oc3cc5ccccc5cc34)nc(-c3cccc4oc5ccccc5c34)n2)c([2H])c1[2H] ; [2H]c1c([2H])c([2H])c(-c2nc(-c3ccc4oc5ccccc5c4c3)nc(-c3cccc4oc5cc6ccccc6cc5c34)n2)c([2H])c1[2H] ; [2H]c1c([2H])c([2H])c(-c2nc(-c3ccc4oc5ccccc5c4c3)nc(-c3ccc4oc5cc6ccccc6cc5c4c3)n2)c([2H])c1[2H]]

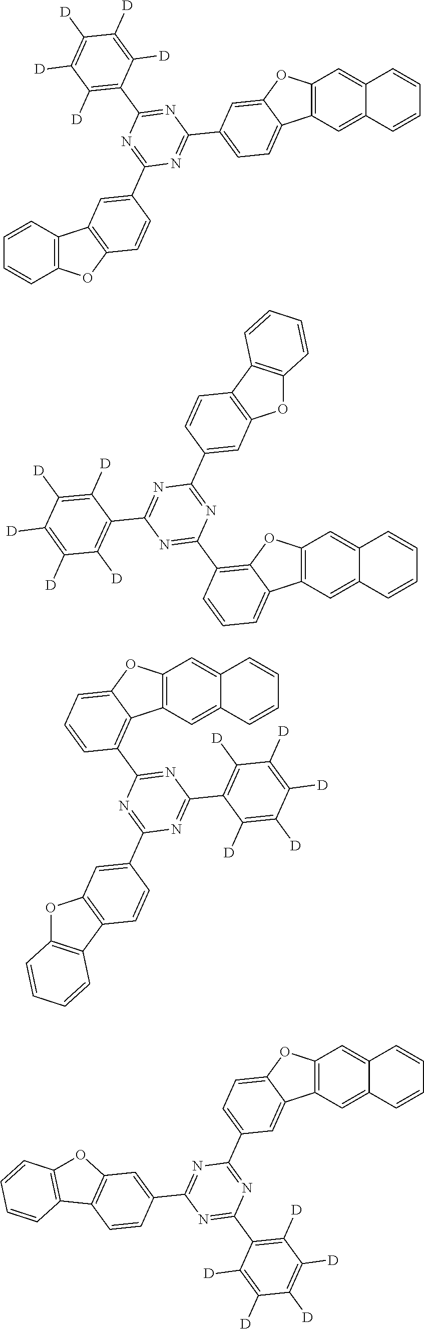

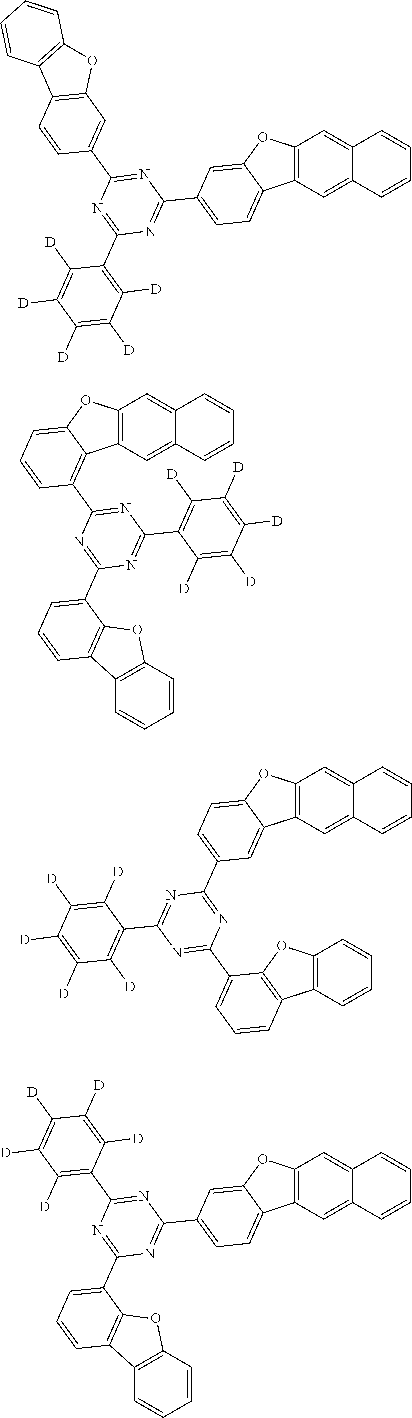 [SMILES: [2H]c1c([2H])c([2H])c(-c2nc(-c3ccc4c(c3)oc3ccccc34)nc(-c3ccc4c(c3)oc3cc5ccccc5cc34)n2)c([2H])c1[2H] ; [2H]c1c([2H])c([2H])c(-c2nc(-c3cccc4c3oc3ccccc34)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cccc4c3oc3ccccc34)n2)c([2H])c1[2H] ; [2H]c1c([2H])c([2H])c(-c2nc(-c3ccc4c(c3)oc3cc5ccccc5cc34)nc(-c3cccc4c3oc3ccccc34)n2)c([2H])c1[2H]]

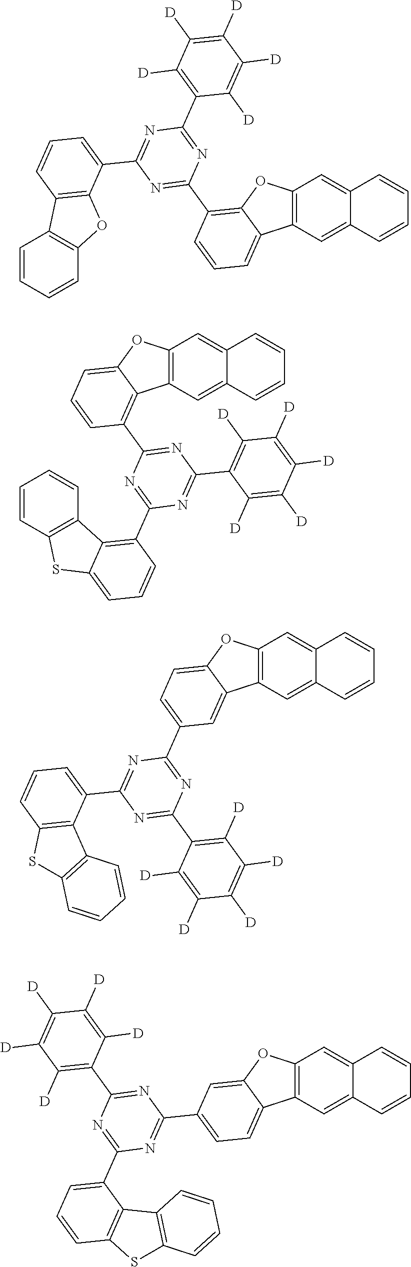 [SMILES: [2H]c1c([2H])c([2H])c(-c2nc(-c3cccc4c3oc3ccccc34)nc(-c3cccc4c3oc3cc5ccccc5cc34)n2)c([2H])c1[2H] ; [2H]c1c([2H])c([2H])c(-c2nc(-c3cccc4oc5cc6ccccc6cc5c34)nc(-c3cccc4sc5ccccc5c34)n2)c([2H])c1[2H] ; [2H]c1c([2H])c([2H])c(-c2nc(-c3ccc4oc5cc6ccccc6cc5c4c3)nc(-c3cccc4sc5ccccc5c34)n2)c([2H])c1[2H] ; [2H]c1c([2H])c([2H])c(-c2nc(-c3ccc4c(c3)oc3cc5ccccc5cc34)nc(-c3cccc4sc5ccccc5c34)n2)c([2H])c1[2H]]

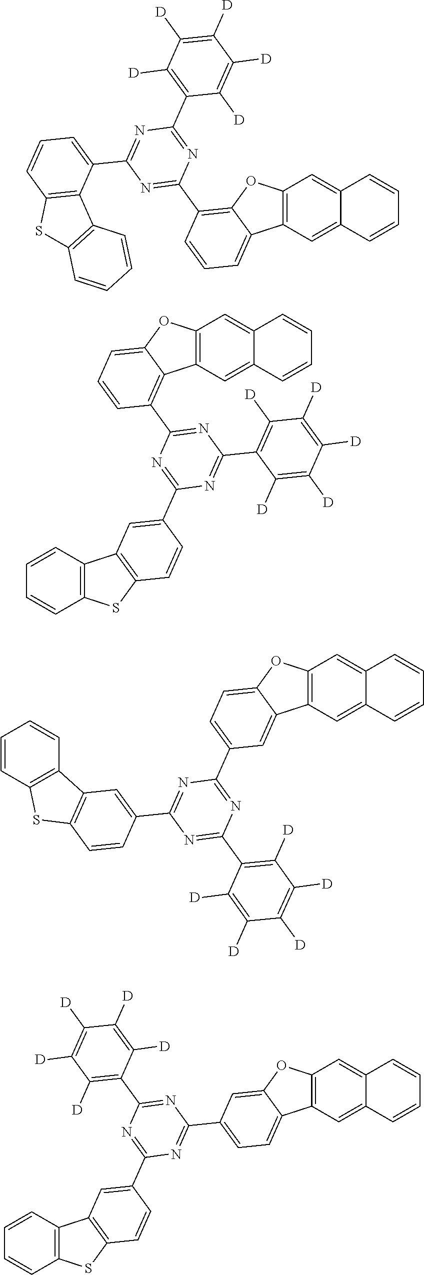 [SMILES: [2H]c1c([2H])c([2H])c(-c2nc(-c3cccc4c3oc3cc5ccccc5cc34)nc(-c3cccc4sc5ccccc5c34)n2)c([2H])c1[2H] ; [2H]c1c([2H])c([2H])c(-c2nc(-c3ccc4sc5ccccc5c4c3)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3ccc4sc5ccccc5c4c3)n2)c([2H])c1[2H] ; [2H]c1c([2H])c([2H])c(-c2nc(-c3ccc4c(c3)oc3cc5ccccc5cc34)nc(-c3ccc4sc5ccccc5c4c3)n2)c([2H])c1[2H]]

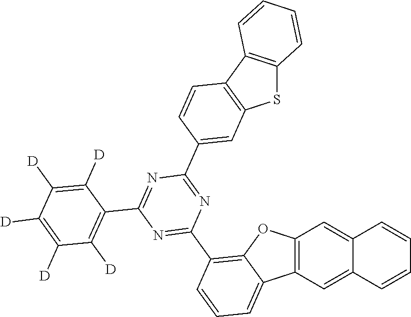 [SMILES: [2H]c1c([2H])c([2H])c(-c2nc(-c3ccc4c(c3)sc3ccccc34)nc(-c3cccc4c3oc3cc5ccccc5cc34)n2)c([2H])c1[2H]]

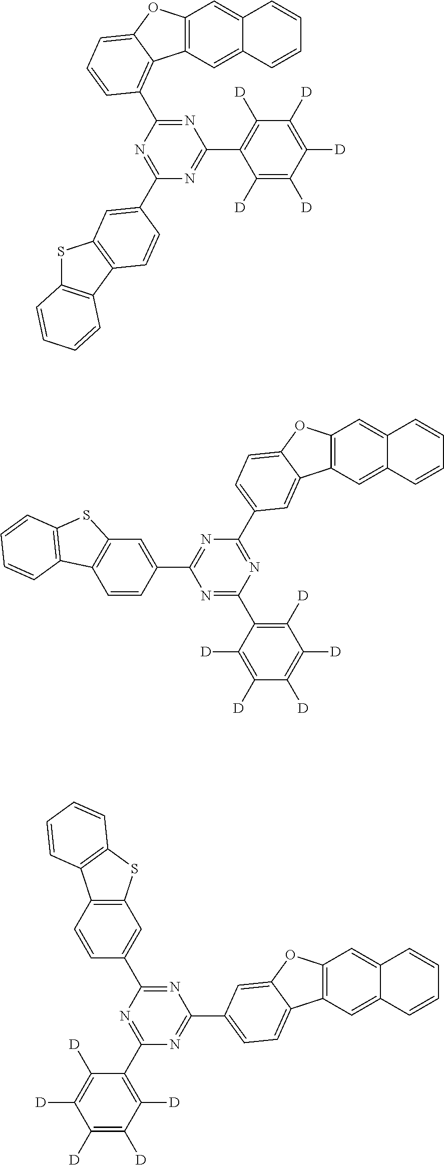 [SMILES: [2H]c1c([2H])c([2H])c(-c2nc(-c3ccc4c(c3)sc3ccccc34)nc(-c3cccc4oc5cc6ccccc6cc5c34)n2)c([2H])c1[2H] ; [2H]c1c([2H])c([2H])c(-c2nc(-c3ccc4c(c3)sc3ccccc34)nc(-c3ccc4oc5cc6ccccc6cc5c4c3)n2)c([2H])c1[2H] ; [2H]c1c([2H])c([2H])c(-c2nc(-c3ccc4c(c3)oc3cc5ccccc5cc34)nc(-c3ccc4c(c3)sc3ccccc34)n2)c([2H])c1[2H]]

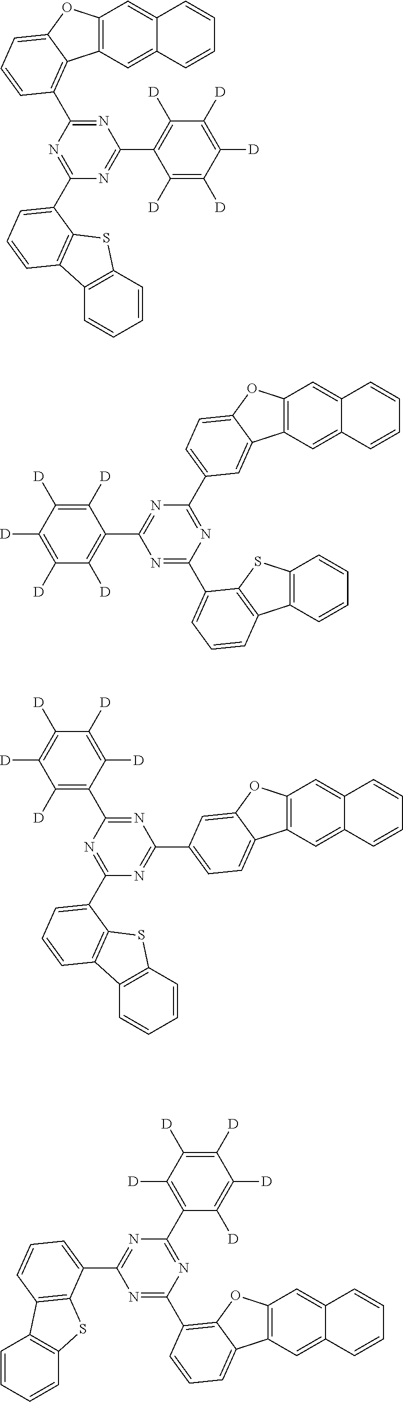 [SMILES: [2H]c1c([2H])c([2H])c(-c2nc(-c3cccc4c3sc3ccccc34)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cccc4c3sc3ccccc34)n2)c([2H])c1[2H] ; [2H]c1c([2H])c([2H])c(-c2nc(-c3ccc4c(c3)oc3cc5ccccc5cc34)nc(-c3cccc4c3sc3ccccc34)n2)c([2H])c1[2H] ; [2H]c1c([2H])c([2H])c(-c2nc(-c3cccc4c3oc3cc5ccccc5cc34)nc(-c3cccc4c3sc3ccccc34)n2)c([2H])c1[2H]]

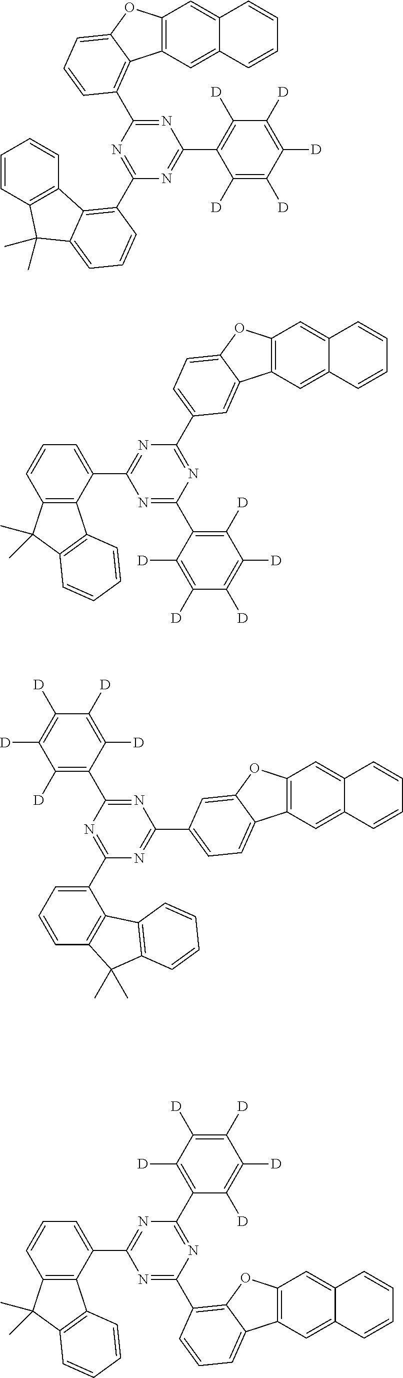 [SMILES: [2H]c1c([2H])c([2H])c(-c2nc(-c3cccc4c3-c3ccccc3C4(C)C)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cccc4c3-c3ccccc3C4(C)C)n2)c([2H])c1[2H] ; [2H]c1c([2H])c([2H])c(-c2nc(-c3ccc4c(c3)oc3cc5ccccc5cc34)nc(-c3cccc4c3-c3ccccc3C4(C)C)n2)c([2H])c1[2H] ; [2H]c1c([2H])c([2H])c(-c2nc(-c3cccc4c3-c3ccccc3C4(C)C)nc(-c3cccc4c3oc3cc5ccccc5cc34)n2)c([2H])c1[2H]]

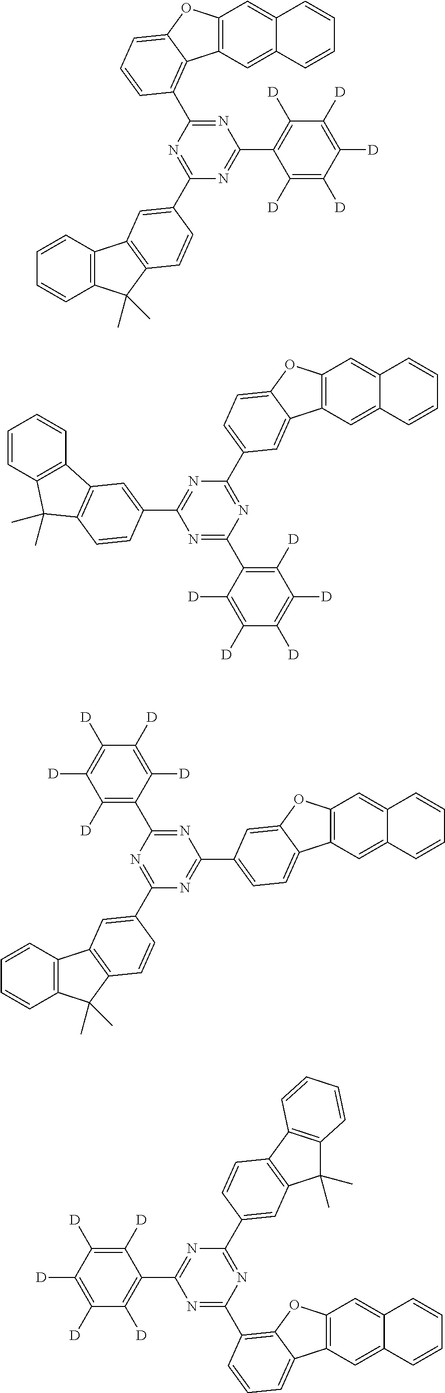 [SMILES: [2H]c1c([2H])c([2H])c(-c2nc(-c3ccc4c(c3)-c3ccccc3C4(C)C)nc(-c3cccc4oc5cc6ccccc6cc5c34)n2)c([2H])c1[2H] ; [2H]c1c([2H])c([2H])c(-c2nc(-c3ccc4c(c3)-c3ccccc3C4(C)C)nc(-c3ccc4oc5cc6ccccc6cc5c4c3)n2)c([2H])c1[2H] ; [2H]c1c([2H])c([2H])c(-c2nc(-c3ccc4c(c3)-c3ccccc3C4(C)C)nc(-c3ccc4c(c3)oc3cc5ccccc5cc34)n2)c([2H])c1[2H] ; [2H]c1c([2H])c([2H])c(-c2nc(-c3ccc4c(c3)C(C)(C)c3ccccc3-4)nc(-c3cccc4c3oc3cc5ccccc5cc34)n2)c([2H])c1[2H]]

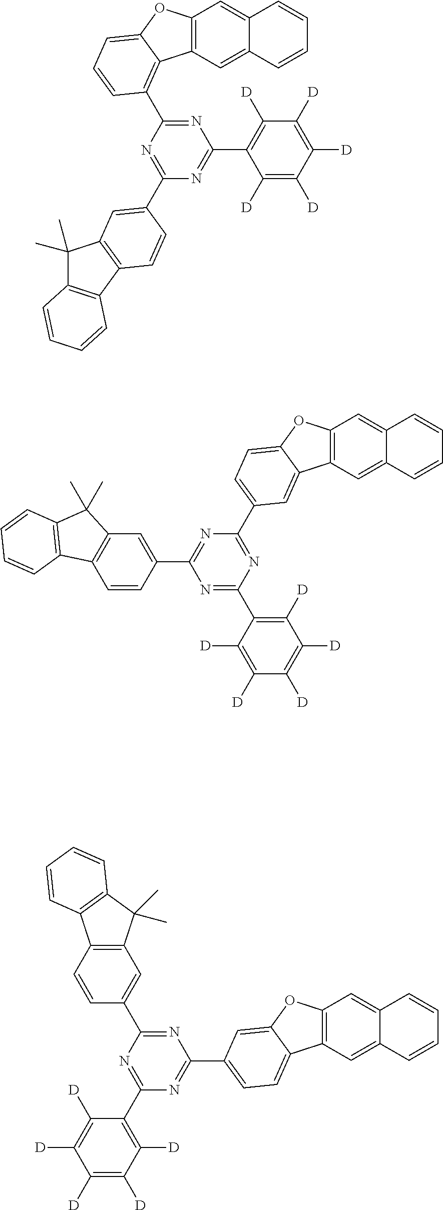 [SMILES: [2H]c1c([2H])c([2H])c(-c2nc(-c3ccc4c(c3)C(C)(C)c3ccccc3-4)nc(-c3cccc4oc5cc6ccccc6cc5c34)n2)c([2H])c1[2H] ; [2H]c1c([2H])c([2H])c(-c2nc(-c3ccc4c(c3)C(C)(C)c3ccccc3-4)nc(-c3ccc4oc5cc6ccccc6cc5c4c3)n2)c([2H])c1[2H] ; [2H]c1c([2H])c([2H])c(-c2nc(-c3ccc4c(c3)C(C)(C)c3ccccc3-4)nc(-c3ccc4c(c3)oc3cc5ccccc5cc34)n2)c([2H])c1[2H]]

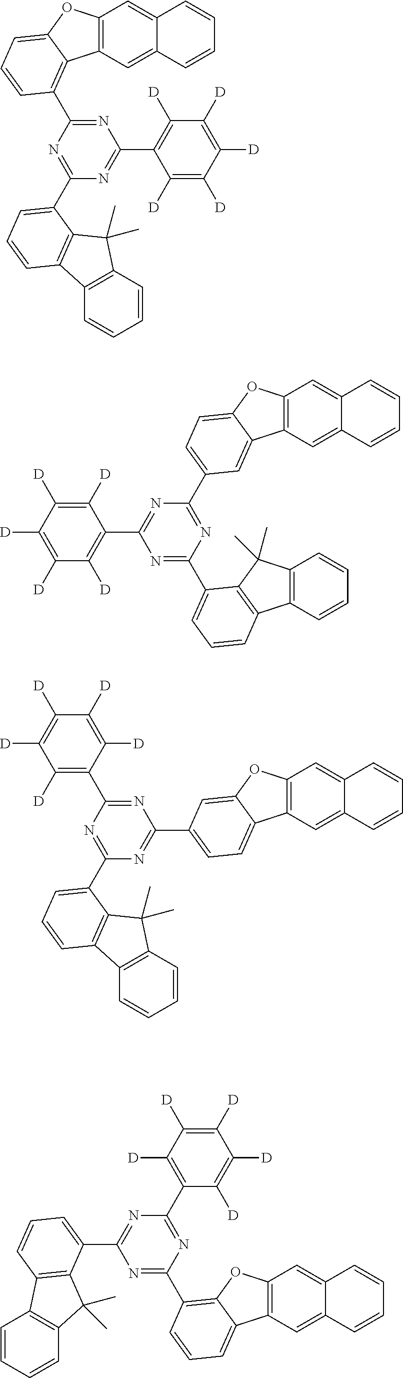 [SMILES: [2H]c1c([2H])c([2H])c(-c2nc(-c3cccc4c3C(C)(C)c3ccccc3-4)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3cccc4c3C(C)(C)c3ccccc3-4)n2)c([2H])c1[2H] ; [2H]c1c([2H])c([2H])c(-c2nc(-c3ccc4c(c3)oc3cc5ccccc5cc34)nc(-c3cccc4c3C(C)(C)c3ccccc3-4)n2)c([2H])c1[2H] ; [2H]c1c([2H])c([2H])c(-c2nc(-c3cccc4c3C(C)(C)c3ccccc3-4)nc(-c3cccc4c3oc3cc5ccccc5cc34)n2)c([2H])c1[2H]]

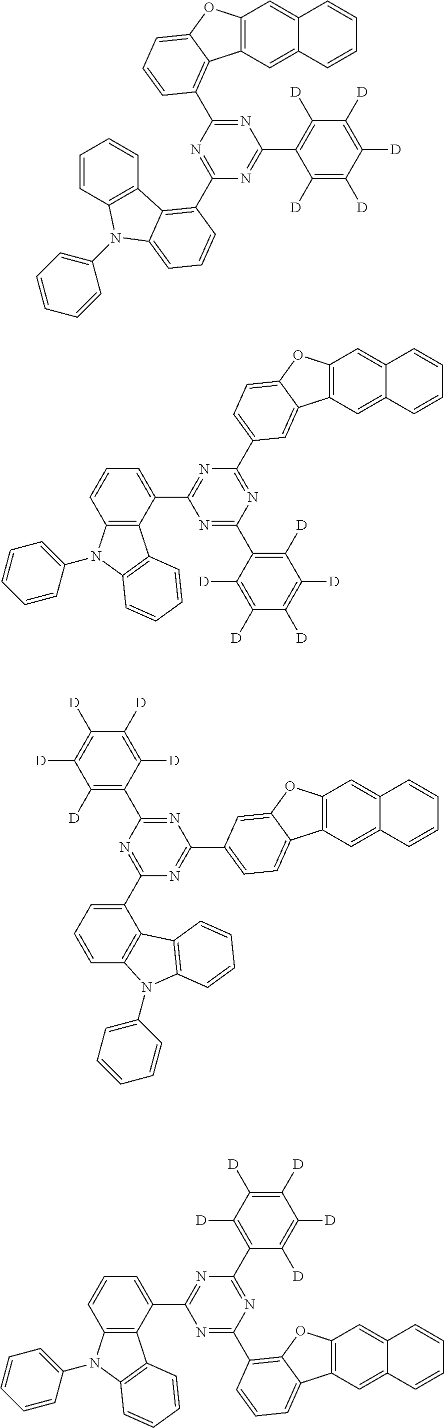 [SMILES: [2H]c1c([2H])c([2H])c(-c2nc(-c3cccc4oc5cc6ccccc6cc5c34)nc(-c3cccc4c3c3ccccc3n4-c3ccccc3)n2)c([2H])c1[2H] ; [2H]c1c([2H])c([2H])c(-c2nc(-c3ccc4oc5cc6ccccc6cc5c4c3)nc(-c3cccc4c3c3ccccc3n4-c3ccccc3)n2)c([2H])c1[2H] ; [2H]c1c([2H])c([2H])c(-c2nc(-c3ccc4c(c3)oc3cc5ccccc5cc34)nc(-c3cccc4c3c3ccccc3n4-c3ccccc3)n2)c([2H])c1[2H] ; [2H]c1c([2H])c([2H])c(-c2nc(-c3cccc4c3oc3cc5ccccc5cc34)nc(-c3cccc4c3c3ccccc3n4-c3ccccc3)n2)c([2H])c1[2H]]

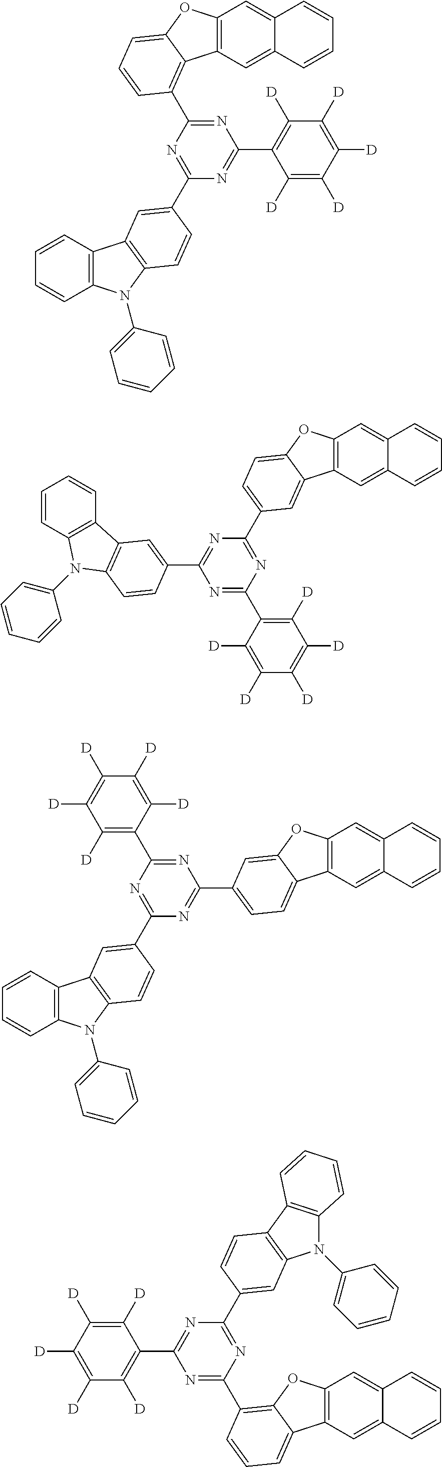 [SMILES: [2H]c1c([2H])c([2H])c(-c2nc(-c3ccc4c(c3)c3ccccc3n4-c3ccccc3)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3ccc4c(c3)c3ccccc3n4-c3ccccc3)n2)c([2H])c1[2H] ; [2H]c1c([2H])c([2H])c(-c2nc(-c3ccc4c(c3)oc3cc5ccccc5cc34)nc(-c3ccc4c(c3)c3ccccc3n4-c3ccccc3)n2)c([2H])c1[2H] ; [2H]c1c([2H])c([2H])c(-c2nc(-c3ccc4c5ccccc5n(-c5ccccc5)c4c3)nc(-c3cccc4c3oc3cc5ccccc5cc34)n2)c([2H])c1[2H]]

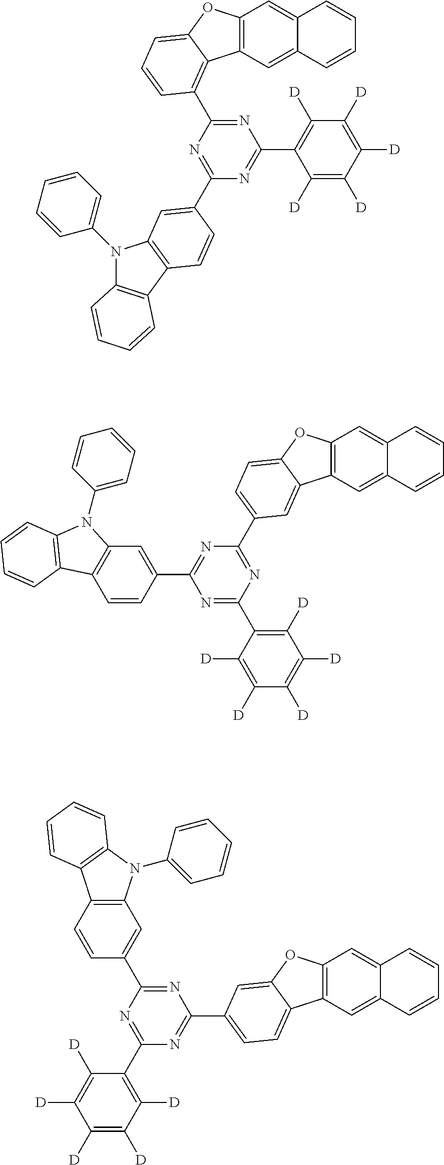 [SMILES: [2H]c1c([2H])c([2H])c(-c2nc(-c3ccc4c5ccccc5n(-c5ccccc5)c4c3)nc(-c3cccc4oc5cc6ccccc6cc5c34)n2)c([2H])c1[2H] ; [2H]c1c([2H])c([2H])c(-c2nc(-c3ccc4oc5cc6ccccc6cc5c4c3)nc(-c3ccc4c5ccccc5n(-c5ccccc5)c4c3)n2)c([2H])c1[2H] ; [2H]c1c([2H])c([2H])c(-c2nc(-c3ccc4c(c3)oc3cc5ccccc5cc34)nc(-c3ccc4c5ccccc5n(-c5ccccc5)c4c3)n2)c([2H])c1[2H]]

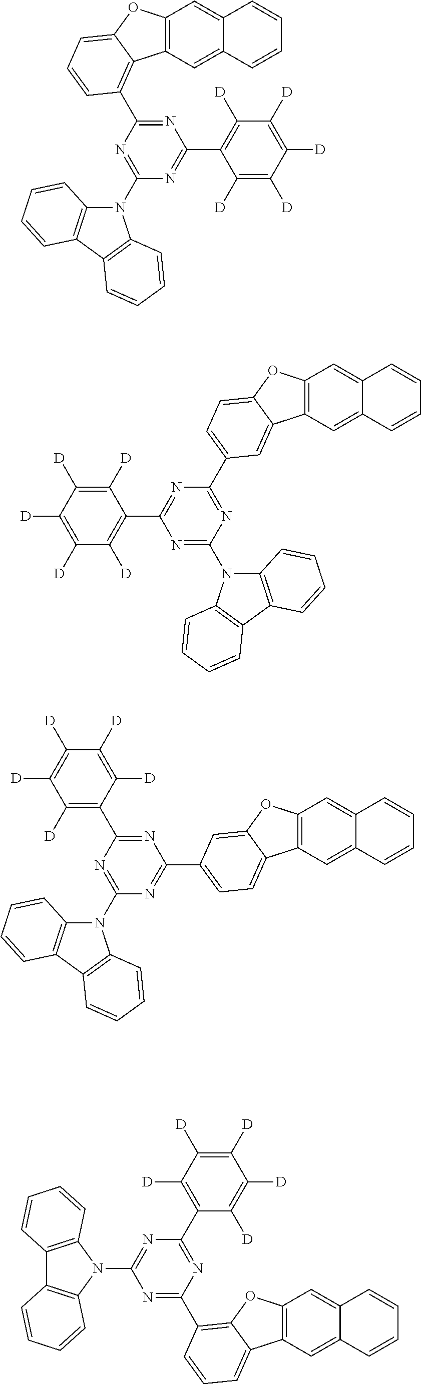 [SMILES: [2H]c1c([2H])c([2H])c(-c2nc(-c3cccc4oc5cc6ccccc6cc5c34)nc(-n3c4ccccc4c4ccccc43)n2)c([2H])c1[2H] ; [2H]c1c([2H])c([2H])c(-c2nc(-c3ccc4oc5cc6ccccc6cc5c4c3)nc(-n3c4ccccc4c4ccccc43)n2)c([2H])c1[2H] ; [2H]c1c([2H])c([2H])c(-c2nc(-c3ccc4c(c3)oc3cc5ccccc5cc34)nc(-n3c4ccccc4c4ccccc43)n2)c([2H])c1[2H] ; [2H]c1c([2H])c([2H])c(-c2nc(-c3cccc4c3oc3cc5ccccc5cc34)nc(-n3c4ccccc4c4ccccc43)n2)c([2H])c1[2H]]

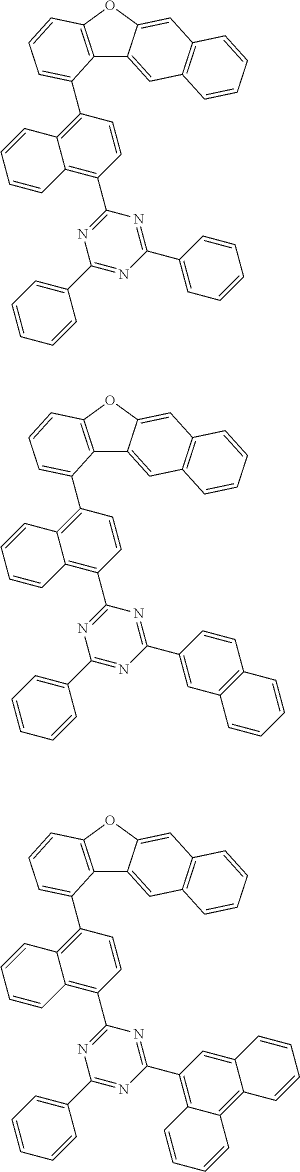 [SMILES: c1ccc(-c2nc(-c3ccccc3)nc(-c3ccc(-c4cccc5oc6cc7ccccc7cc6c45)c4ccccc34)n2)cc1 ; c1ccc(-c2nc(-c3ccc4ccccc4c3)nc(-c3ccc(-c4cccc5oc6cc7ccccc7cc6c45)c4ccccc34)n2)cc1 ; c1ccc(-c2nc(-c3ccc(-c4cccc5oc6cc7ccccc7cc6c45)c4ccccc34)nc(-c3cc4ccccc4c4ccccc34)n2)cc1]

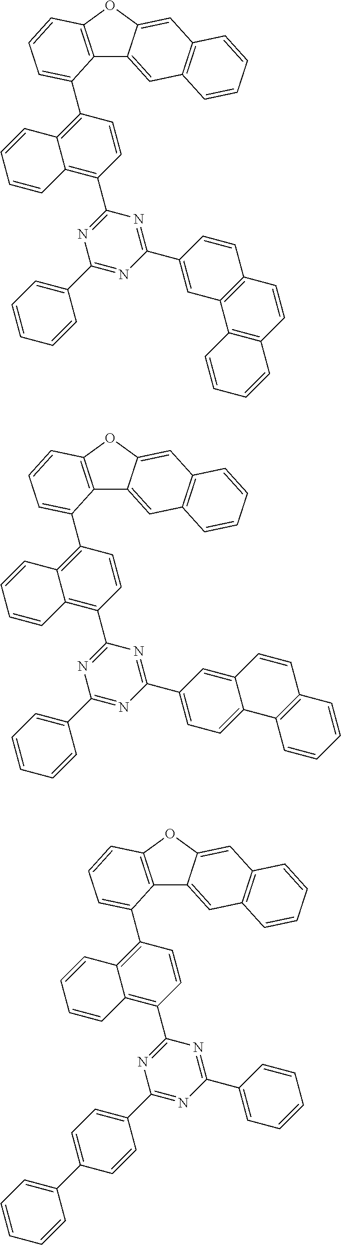 [SMILES: c1ccc(-c2nc(-c3ccc4ccc5ccccc5c4c3)nc(-c3ccc(-c4cccc5oc6cc7ccccc7cc6c45)c4ccccc34)n2)cc1 ; c1ccc(-c2nc(-c3ccc4c(ccc5ccccc54)c3)nc(-c3ccc(-c4cccc5oc6cc7ccccc7cc6c45)c4ccccc34)n2)cc1 ; c1ccc(-c2ccc(-c3nc(-c4ccccc4)nc(-c4ccc(-c5cccc6oc7cc8ccccc8cc7c56)c5ccccc45)n3)cc2)cc1]

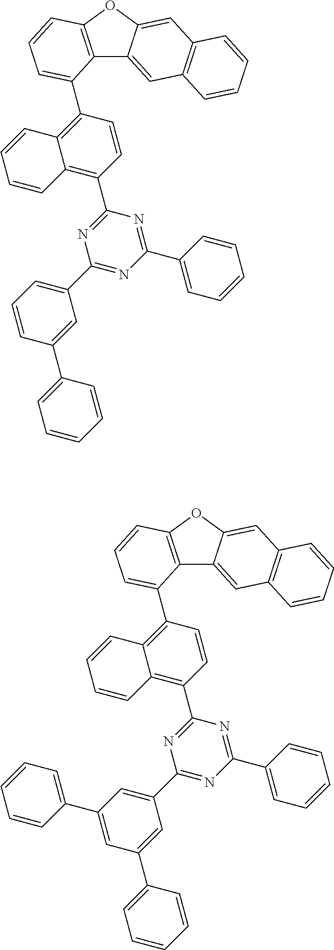 [SMILES: c1ccc(-c2cccc(-c3nc(-c4ccccc4)nc(-c4ccc(-c5cccc6oc7cc8ccccc8cc7c56)c5ccccc45)n3)c2)cc1 ; c1ccc(-c2cc(-c3ccccc3)cc(-c3nc(-c4ccccc4)nc(-c4ccc(-c5cccc6oc7cc8ccccc8cc7c56)c5ccccc45)n3)c2)cc1]

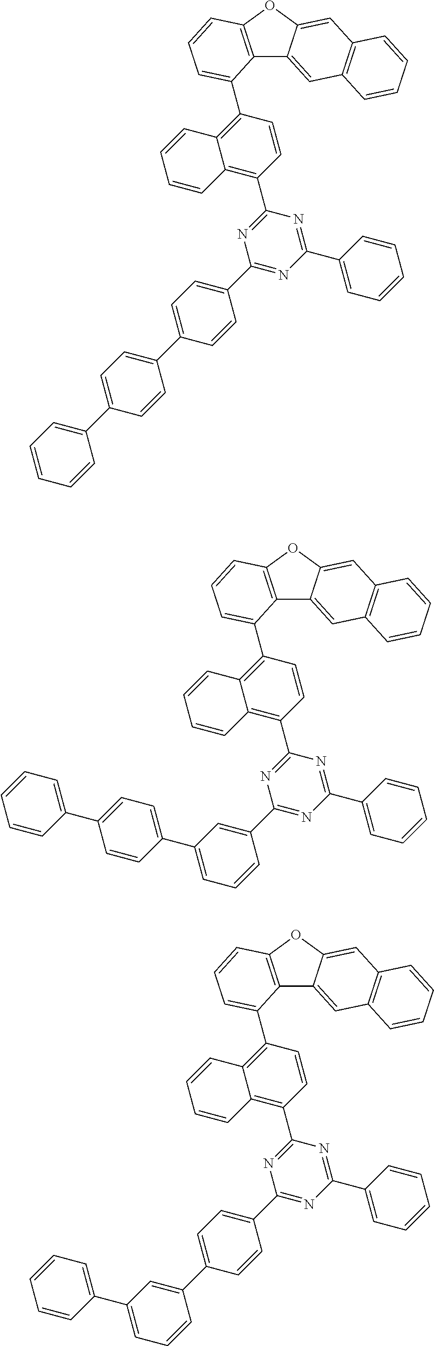 [SMILES: c1ccc(-c2ccc(-c3ccc(-c4nc(-c5ccccc5)nc(-c5ccc(-c6cccc7oc8cc9ccccc9cc8c67)c6ccccc56)n4)cc3)cc2)cc1 ; c1ccc(-c2ccc(-c3cccc(-c4nc(-c5ccccc5)nc(-c5ccc(-c6cccc7oc8cc9ccccc9cc8c67)c6ccccc56)n4)c3)cc2)cc1 ; c1ccc(-c2cccc(-c3ccc(-c4nc(-c5ccccc5)nc(-c5ccc(-c6cccc7oc8cc9ccccc9cc8c67)c6ccccc56)n4)cc3)c2)cc1]

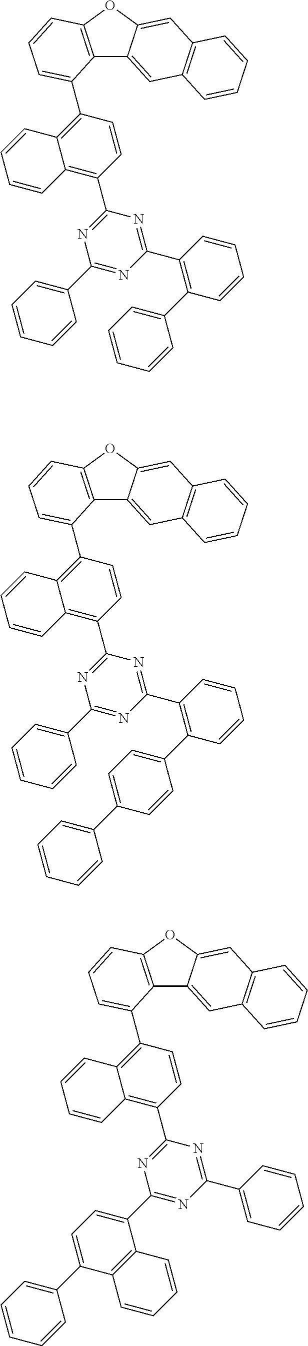 [SMILES: c1ccc(-c2nc(-c3ccccc3-c3ccccc3)nc(-c3ccc(-c4cccc5oc6cc7ccccc7cc6c45)c4ccccc34)n2)cc1 ; c1ccc(-c2ccc(-c3ccccc3-c3nc(-c4ccccc4)nc(-c4ccc(-c5cccc6oc7cc8ccccc8cc7c56)c5ccccc45)n3)cc2)cc1 ; c1ccc(-c2nc(-c3ccc(-c4ccccc4)c4ccccc34)nc(-c3ccc(-c4cccc5oc6cc7ccccc7cc6c45)c4ccccc34)n2)cc1]

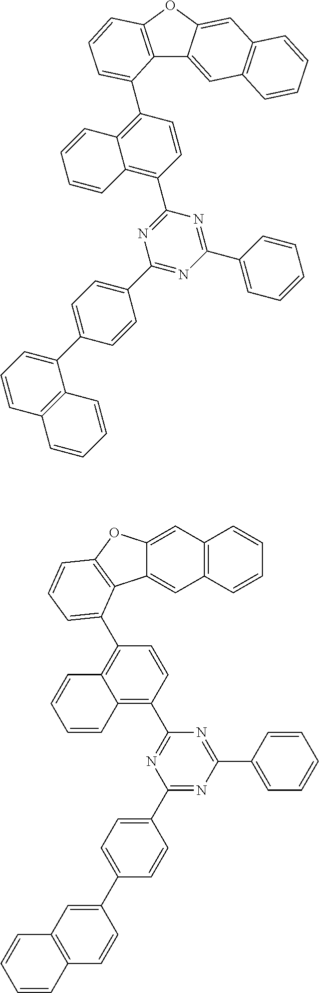 [SMILES: c1ccc(-c2nc(-c3ccc(-c4cccc5ccccc45)cc3)nc(-c3ccc(-c4cccc5oc6cc7ccccc7cc6c45)c4ccccc34)n2)cc1 ; c1ccc(-c2nc(-c3ccc(-c4ccc5ccccc5c4)cc3)nc(-c3ccc(-c4cccc5oc6cc7ccccc7cc6c45)c4ccccc34)n2)cc1]

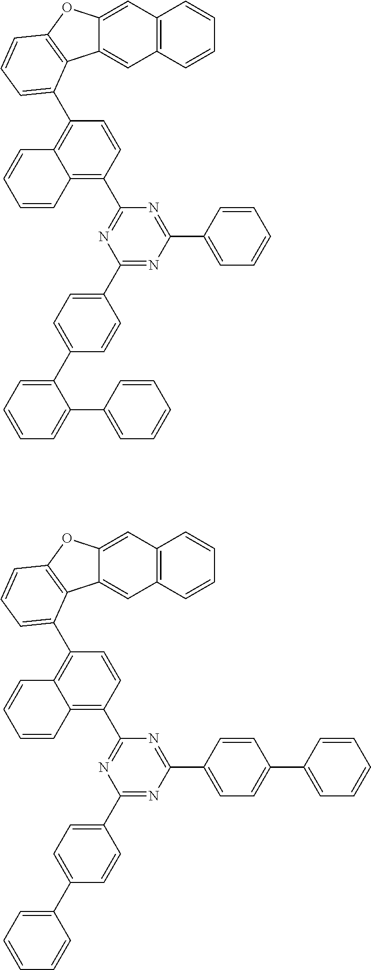 [SMILES: c1ccc(-c2nc(-c3ccc(-c4ccccc4-c4ccccc4)cc3)nc(-c3ccc(-c4cccc5oc6cc7ccccc7cc6c45)c4ccccc34)n2)cc1 ; c1ccc(-c2ccc(-c3nc(-c4ccc(-c5ccccc5)cc4)nc(-c4ccc(-c5cccc6oc7cc8ccccc8cc7c56)c5ccccc45)n3)cc2)cc1]

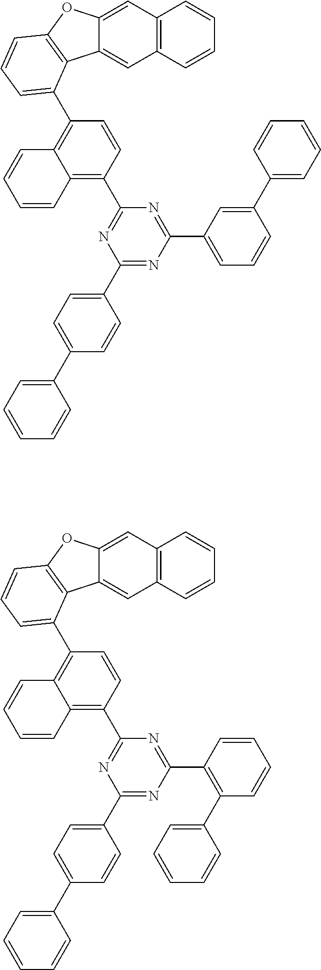 [SMILES: c1ccc(-c2ccc(-c3nc(-c4cccc(-c5ccccc5)c4)nc(-c4ccc(-c5cccc6oc7cc8ccccc8cc7c56)c5ccccc45)n3)cc2)cc1 ; c1ccc(-c2ccc(-c3nc(-c4ccccc4-c4ccccc4)nc(-c4ccc(-c5cccc6oc7cc8ccccc8cc7c56)c5ccccc45)n3)cc2)cc1]

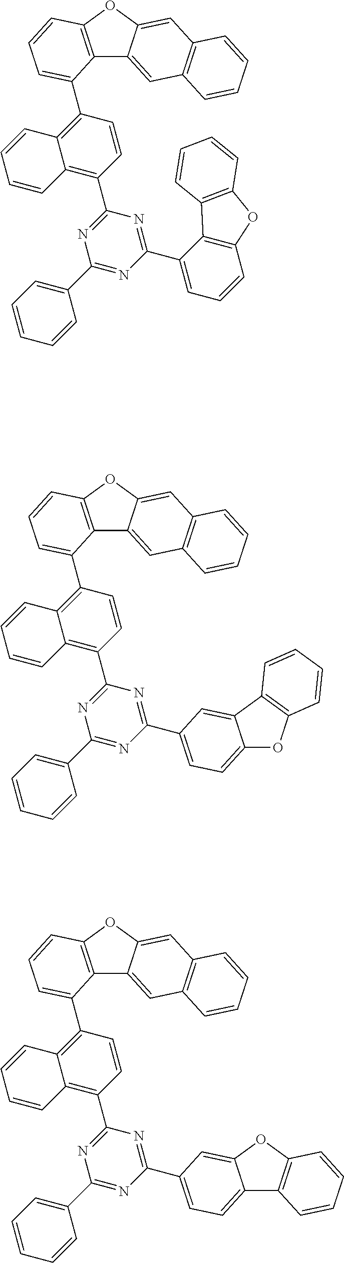 [SMILES: c1ccc(-c2nc(-c3ccc(-c4cccc5oc6cc7ccccc7cc6c45)c4ccccc34)nc(-c3cccc4oc5ccccc5c34)n2)cc1 ; c1ccc(-c2nc(-c3ccc4oc5ccccc5c4c3)nc(-c3ccc(-c4cccc5oc6cc7ccccc7cc6c45)c4ccccc34)n2)cc1 ; c1ccc(-c2nc(-c3ccc4c(c3)oc3ccccc34)nc(-c3ccc(-c4cccc5oc6cc7ccccc7cc6c45)c4ccccc34)n2)cc1]

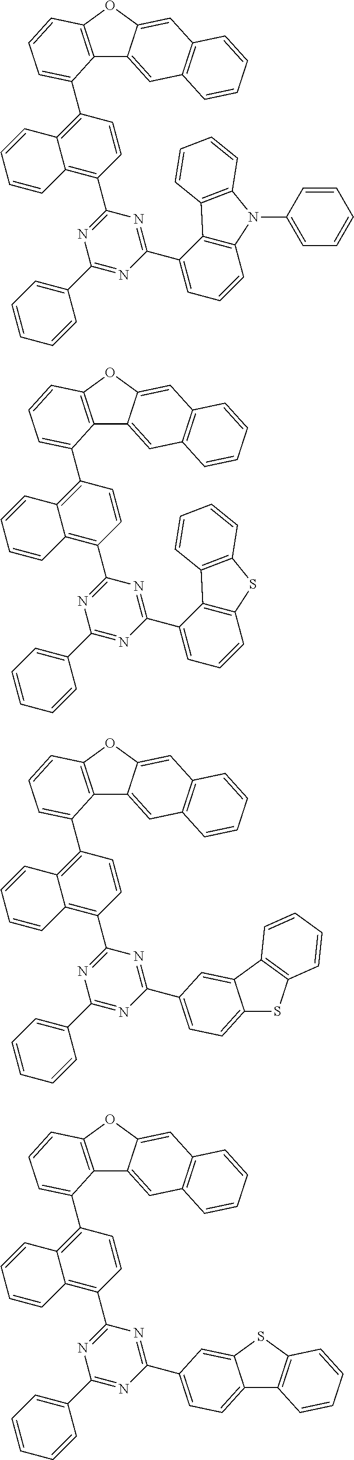

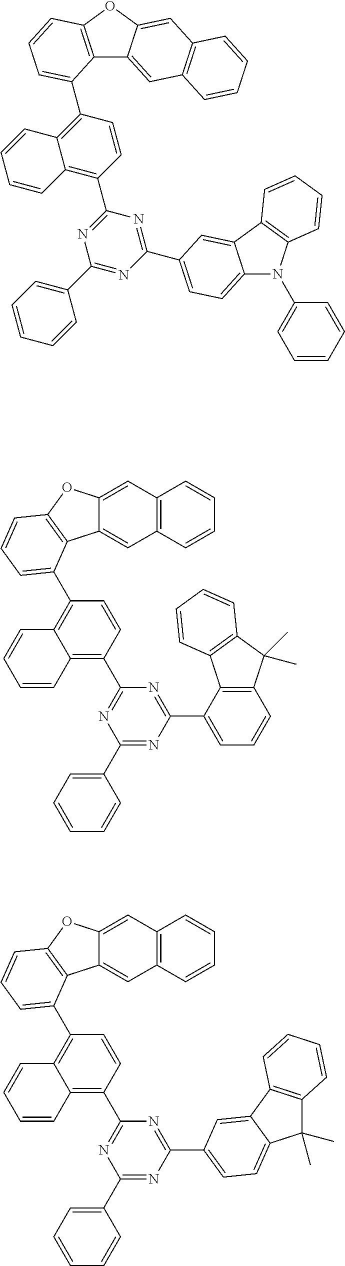 [SMILES: c1ccc(-c2nc(-c3ccc4c(c3)c3ccccc3n4-c3ccccc3)nc(-c3ccc(-c4cccc5oc6cc7ccccc7cc6c45)c4ccccc34)n2)cc1 ; CC1(C)c2ccccc2-c2c(-c3nc(-c4ccccc4)nc(-c4ccc(-c5cccc6oc7cc8ccccc8cc7c56)c5ccccc45)n3)cccc21 ; CC1(C)c2ccccc2-c2cc(-c3nc(-c4ccccc4)nc(-c4ccc(-c5cccc6oc7cc8ccccc8cc7c56)c5ccccc45)n3)ccc21]

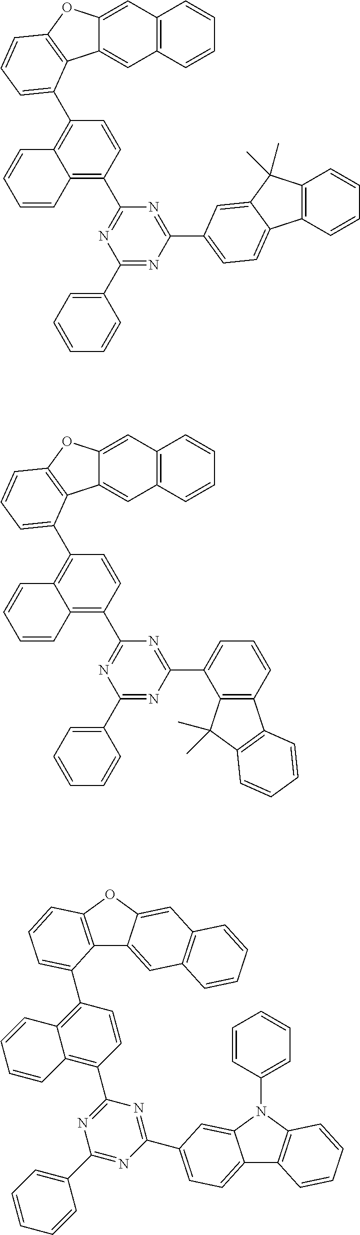 [SMILES: CC1(C)c2ccccc2-c2ccc(-c3nc(-c4ccccc4)nc(-c4ccc(-c5cccc6oc7cc8ccccc8cc7c56)c5ccccc45)n3)cc21 ; CC1(C)c2ccccc2-c2cccc(-c3nc(-c4ccccc4)nc(-c4ccc(-c5cccc6oc7cc8ccccc8cc7c56)c5ccccc45)n3)c21 ; c1ccc(-c2nc(-c3ccc4c5ccccc5n(-c5ccccc5)c4c3)nc(-c3ccc(-c4cccc5oc6cc7ccccc7cc6c45)c4ccccc34)n2)cc1]

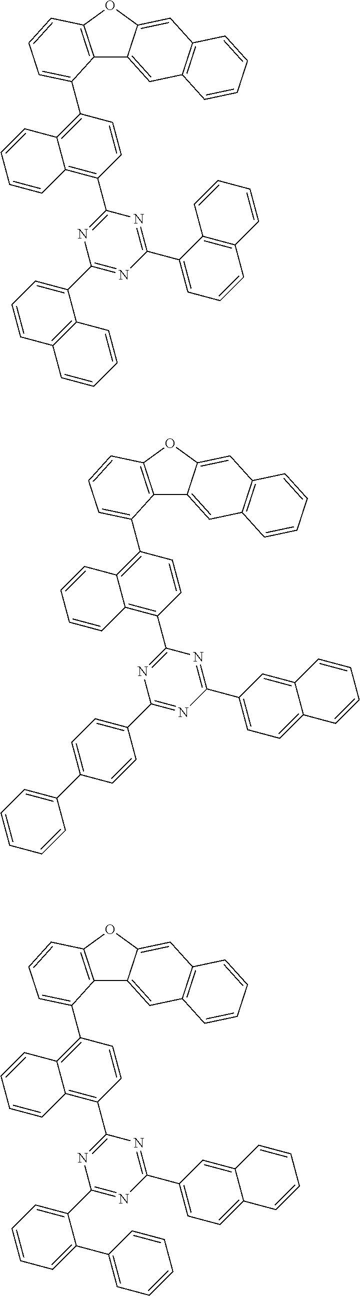 [SMILES: c1ccc2cc3c(cc2c1)oc1cccc(-c2ccc(-c4nc(-c5cccc6ccccc56)nc(-c5cccc6ccccc56)n4)c4ccccc24)c13 ; c1ccc(-c2ccc(-c3nc(-c4ccc5ccccc5c4)nc(-c4ccc(-c5cccc6oc7cc8ccccc8cc7c56)c5ccccc45)n3)cc2)cc1 ; c1ccc(-c2ccccc2-c2nc(-c3ccc4ccccc4c3)nc(-c3ccc(-c4cccc5oc6cc7ccccc7cc6c45)c4ccccc34)n2)cc1]

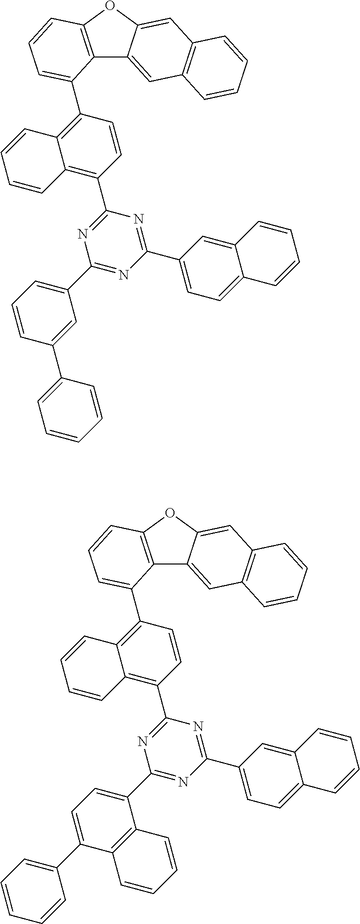 [SMILES: c1ccc(-c2cccc(-c3nc(-c4ccc5ccccc5c4)nc(-c4ccc(-c5cccc6oc7cc8ccccc8cc7c56)c5ccccc45)n3)c2)cc1 ; c1ccc(-c2ccc(-c3nc(-c4ccc5ccccc5c4)nc(-c4ccc(-c5cccc6oc7cc8ccccc8cc7c56)c5ccccc45)n3)c3ccccc23)cc1]

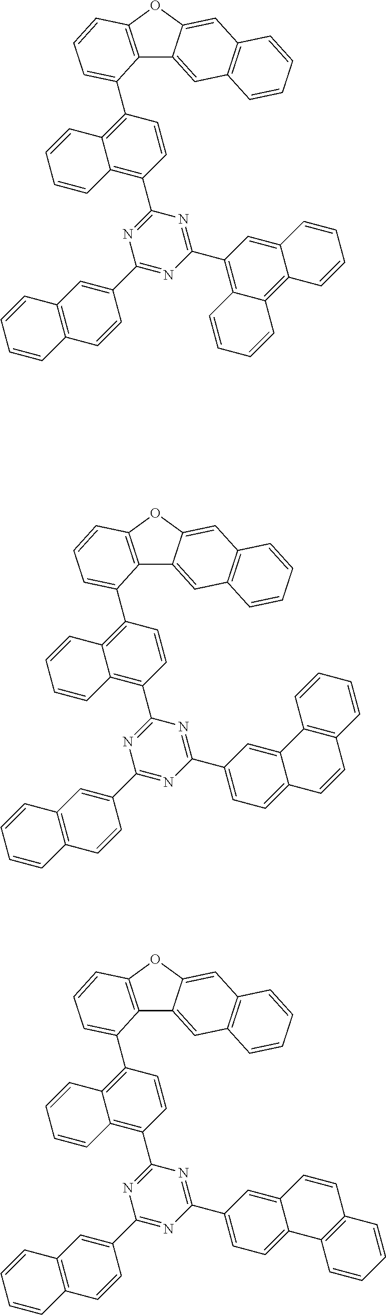 [SMILES: c1ccc2cc(-c3nc(-c4ccc(-c5cccc6oc7cc8ccccc8cc7c56)c5ccccc45)nc(-c4cc5ccccc5c5ccccc45)n3)ccc2c1 ; c1ccc2cc(-c3nc(-c4ccc5ccc6ccccc6c5c4)nc(-c4ccc(-c5cccc6oc7cc8ccccc8cc7c56)c5ccccc45)n3)ccc2c1 ; c1ccc2cc(-c3nc(-c4ccc5c(ccc6ccccc65)c4)nc(-c4ccc(-c5cccc6oc7cc8ccccc8cc7c56)c5ccccc45)n3)ccc2c1]

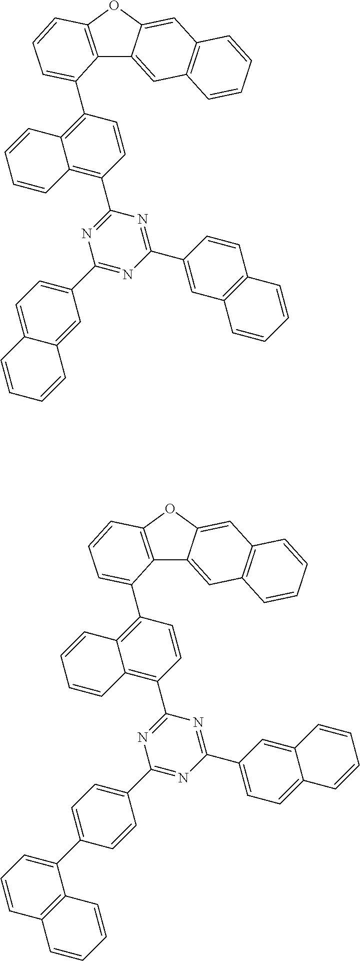 [SMILES: c1ccc2cc(-c3nc(-c4ccc5ccccc5c4)nc(-c4ccc(-c5cccc6oc7cc8ccccc8cc7c56)c5ccccc45)n3)ccc2c1 ; c1ccc2cc(-c3nc(-c4ccc(-c5cccc6ccccc56)cc4)nc(-c4ccc(-c5cccc6oc7cc8ccccc8cc7c56)c5ccccc45)n3)ccc2c1]

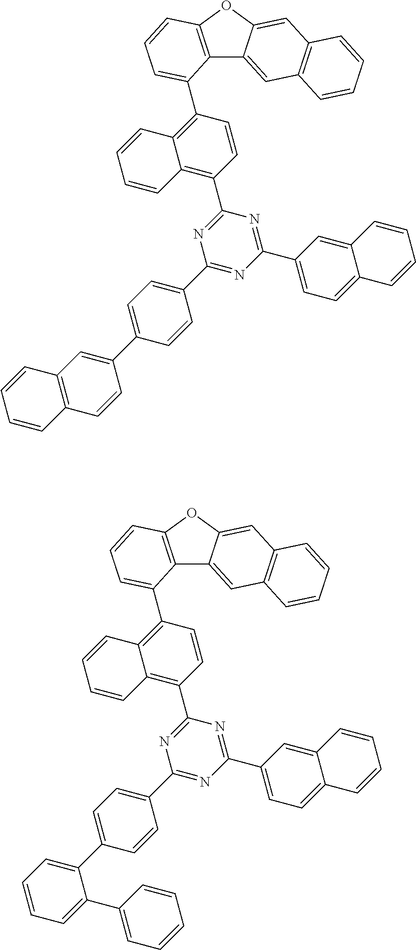 [SMILES: c1ccc2cc(-c3ccc(-c4nc(-c5ccc6ccccc6c5)nc(-c5ccc(-c6cccc7oc8cc9ccccc9cc8c67)c6ccccc56)n4)cc3)ccc2c1 ; c1ccc(-c2ccccc2-c2ccc(-c3nc(-c4ccc5ccccc5c4)nc(-c4ccc(-c5cccc6oc7cc8ccccc8cc7c56)c5ccccc45)n3)cc2)cc1]

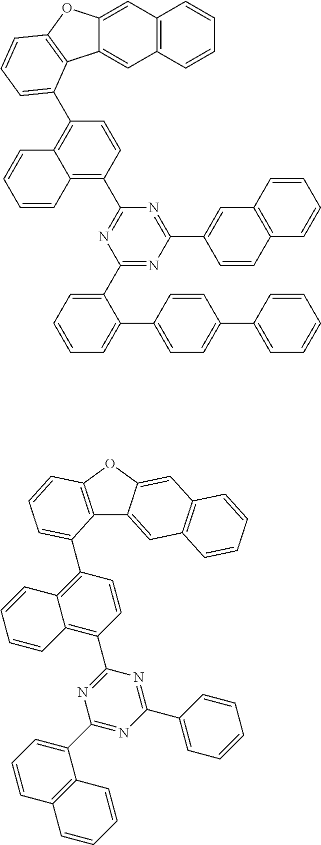 [SMILES: c1ccc(-c2ccc(-c3ccccc3-c3nc(-c4ccc5ccccc5c4)nc(-c4ccc(-c5cccc6oc7cc8ccccc8cc7c56)c5ccccc45)n3)cc2)cc1 ; c1ccc(-c2nc(-c3cccc4ccccc34)nc(-c3ccc(-c4cccc5oc6cc7ccccc7cc6c45)c4ccccc34)n2)cc1]

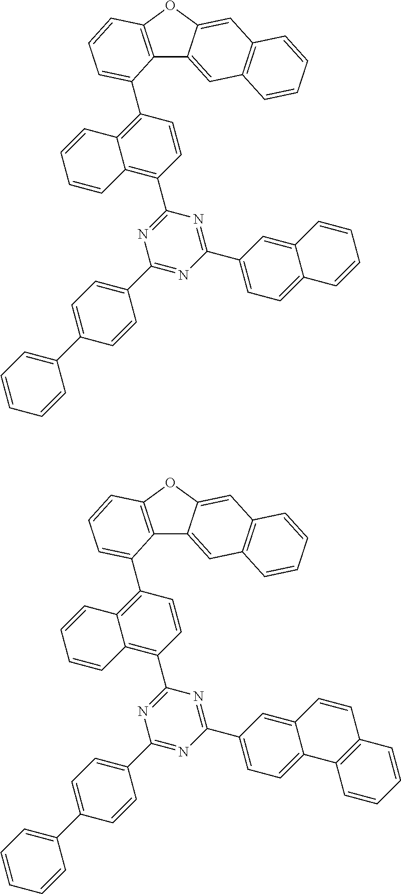 [SMILES: c1ccc(-c2ccc(-c3nc(-c4ccc5ccccc5c4)nc(-c4ccc(-c5cccc6oc7cc8ccccc8cc7c56)c5ccccc45)n3)cc2)cc1 ; c1ccc(-c2ccc(-c3nc(-c4ccc5c(ccc6ccccc65)c4)nc(-c4ccc(-c5cccc6oc7cc8ccccc8cc7c56)c5ccccc45)n3)cc2)cc1]

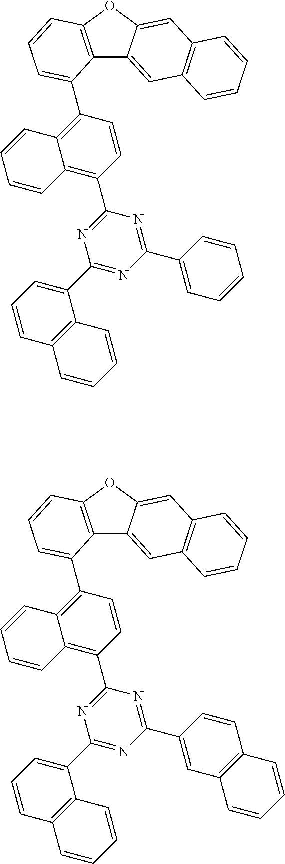 [SMILES: c1ccc(-c2nc(-c3cccc4ccccc34)nc(-c3ccc(-c4cccc5oc6cc7ccccc7cc6c45)c4ccccc34)n2)cc1 ; c1ccc2cc(-c3nc(-c4cccc5ccccc45)nc(-c4ccc(-c5cccc6oc7cc8ccccc8cc7c56)c5ccccc45)n3)ccc2c1]

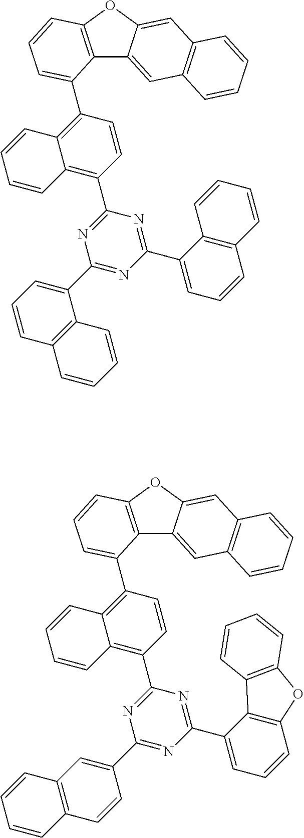 [SMILES: c1ccc2cc3c(cc2c1)oc1cccc(-c2ccc(-c4nc(-c5cccc6ccccc56)nc(-c5cccc6ccccc56)n4)c4ccccc24)c13 ; c1ccc2cc(-c3nc(-c4ccc(-c5cccc6oc7cc8ccccc8cc7c56)c5ccccc45)nc(-c4cccc5oc6ccccc6c45)n3)ccc2c1]

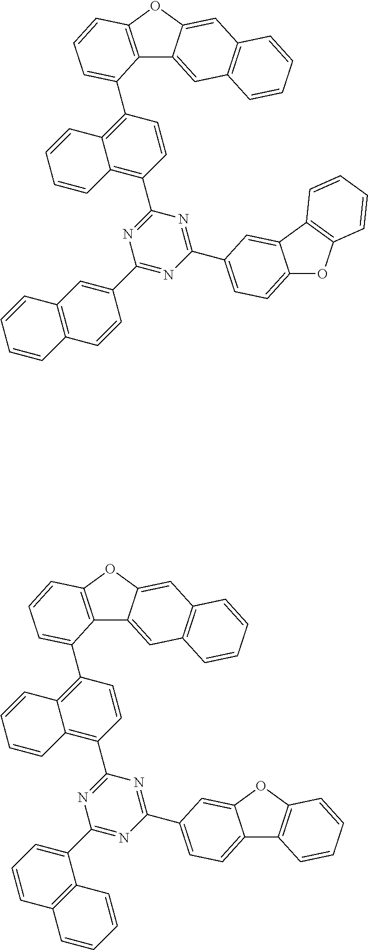 [SMILES: c1ccc2cc(-c3nc(-c4ccc5oc6ccccc6c5c4)nc(-c4ccc(-c5cccc6oc7cc8ccccc8cc7c56)c5ccccc45)n3)ccc2c1 ; c1ccc2cc3c(cc2c1)oc1cccc(-c2ccc(-c4nc(-c5ccc6c(c5)oc5ccccc56)nc(-c5cccc6ccccc56)n4)c4ccccc24)c13]

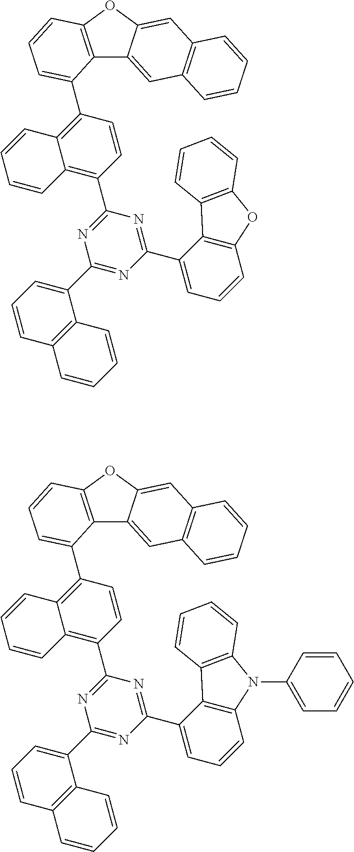 [SMILES: c1ccc2cc3c(cc2c1)oc1cccc(-c2ccc(-c4nc(-c5cccc6ccccc56)nc(-c5cccc6oc7ccccc7c56)n4)c4ccccc24)c13 ; c1ccc(-n2c3ccccc3c3c(-c4nc(-c5cccc6ccccc56)nc(-c5ccc(-c6cccc7oc8cc9ccccc9cc8c67)c6ccccc56)n4)cccc32)cc1]

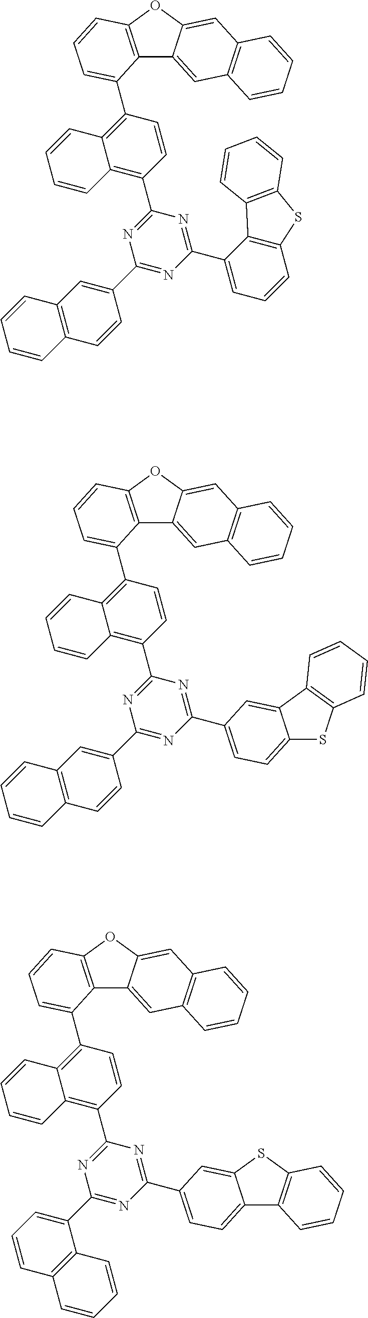 [SMILES: c1ccc2cc(-c3nc(-c4ccc(-c5cccc6oc7cc8ccccc8cc7c56)c5ccccc45)nc(-c4cccc5sc6ccccc6c45)n3)ccc2c1 ; c1ccc2cc(-c3nc(-c4ccc5sc6ccccc6c5c4)nc(-c4ccc(-c5cccc6oc7cc8ccccc8cc7c56)c5ccccc45)n3)ccc2c1 ; c1ccc2cc3c(cc2c1)oc1cccc(-c2ccc(-c4nc(-c5ccc6c(c5)sc5ccccc56)nc(-c5cccc6ccccc56)n4)c4ccccc24)c13]

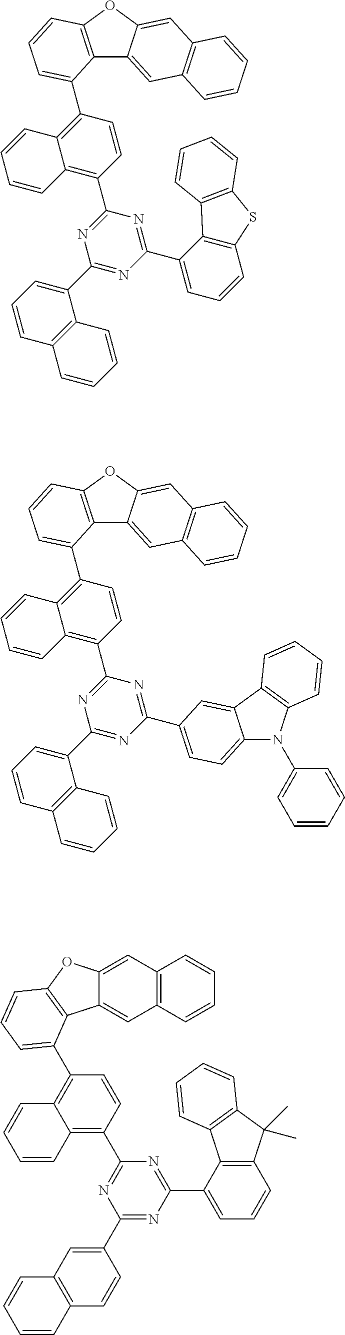 [SMILES: c1ccc2cc3c(cc2c1)oc1cccc(-c2ccc(-c4nc(-c5cccc6ccccc56)nc(-c5cccc6sc7ccccc7c56)n4)c4ccccc24)c13 ; c1ccc(-n2c3ccccc3c3cc(-c4nc(-c5cccc6ccccc56)nc(-c5ccc(-c6cccc7oc8cc9ccccc9cc8c67)c6ccccc56)n4)ccc32)cc1 ; CC1(C)c2ccccc2-c2c(-c3nc(-c4ccc5ccccc5c4)nc(-c4ccc(-c5cccc6oc7cc8ccccc8cc7c56)c5ccccc45)n3)cccc21]

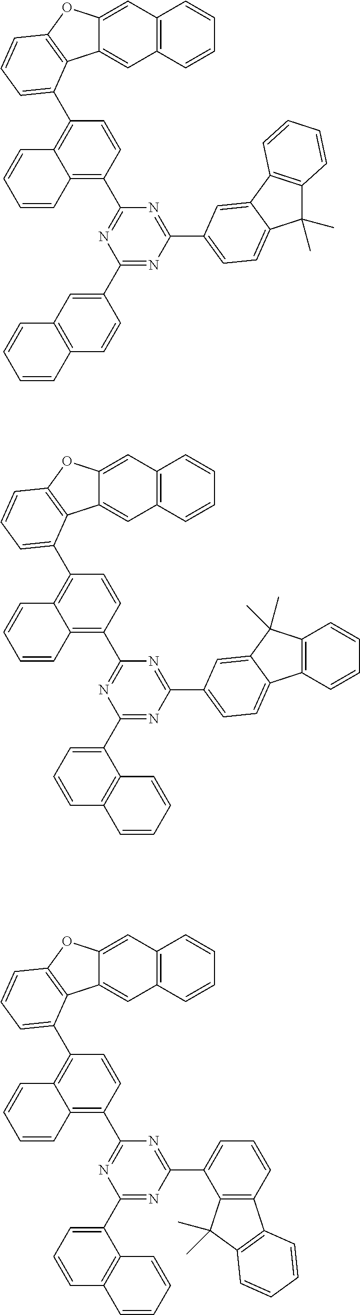 [SMILES: CC1(C)c2ccccc2-c2cc(-c3nc(-c4ccc5ccccc5c4)nc(-c4ccc(-c5cccc6oc7cc8ccccc8cc7c56)c5ccccc45)n3)ccc21 ; CC1(C)c2ccccc2-c2ccc(-c3nc(-c4cccc5ccccc45)nc(-c4ccc(-c5cccc6oc7cc8ccccc8cc7c56)c5ccccc45)n3)cc21 ; CC1(C)c2ccccc2-c2cccc(-c3nc(-c4cccc5ccccc45)nc(-c4ccc(-c5cccc6oc7cc8ccccc8cc7c56)c5ccccc45)n3)c21]

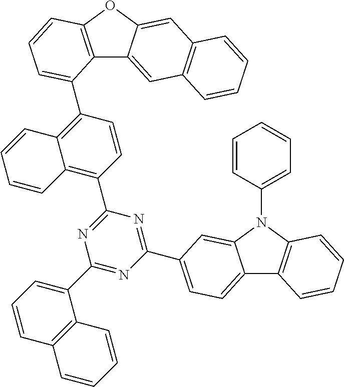 [SMILES: c1ccc(-n2c3ccccc3c3ccc(-c4nc(-c5cccc6ccccc56)nc(-c5ccc(-c6cccc7oc8cc9ccccc9cc8c67)c6ccccc56)n4)cc32)cc1]

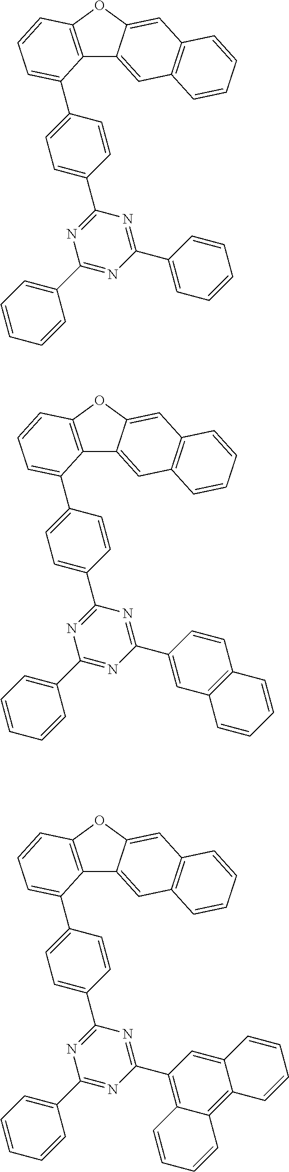 [SMILES: c1ccc(-c2nc(-c3ccccc3)nc(-c3ccc(-c4cccc5oc6cc7ccccc7cc6c45)cc3)n2)cc1 ; c1ccc(-c2nc(-c3ccc(-c4cccc5oc6cc7ccccc7cc6c45)cc3)nc(-c3ccc4ccccc4c3)n2)cc1 ; c1ccc(-c2nc(-c3ccc(-c4cccc5oc6cc7ccccc7cc6c45)cc3)nc(-c3cc4ccccc4c4ccccc34)n2)cc1]

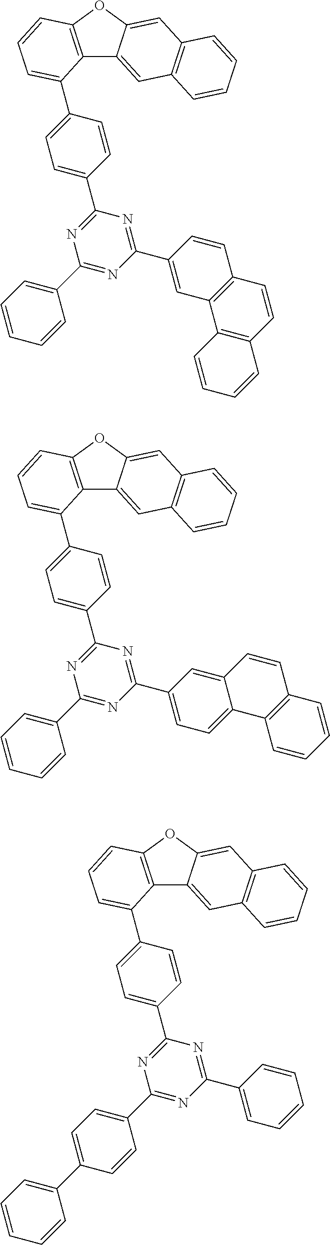 [SMILES: c1ccc(-c2nc(-c3ccc(-c4cccc5oc6cc7ccccc7cc6c45)cc3)nc(-c3ccc4ccc5ccccc5c4c3)n2)cc1 ; c1ccc(-c2nc(-c3ccc(-c4cccc5oc6cc7ccccc7cc6c45)cc3)nc(-c3ccc4c(ccc5ccccc54)c3)n2)cc1 ; c1ccc(-c2ccc(-c3nc(-c4ccccc4)nc(-c4ccc(-c5cccc6oc7cc8ccccc8cc7c56)cc4)n3)cc2)cc1]

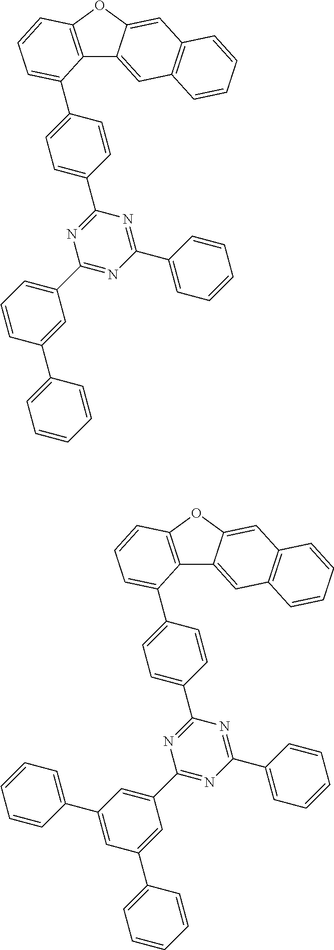 [SMILES: c1ccc(-c2cccc(-c3nc(-c4ccccc4)nc(-c4ccc(-c5cccc6oc7cc8ccccc8cc7c56)cc4)n3)c2)cc1 ; c1ccc(-c2cc(-c3ccccc3)cc(-c3nc(-c4ccccc4)nc(-c4ccc(-c5cccc6oc7cc8ccccc8cc7c56)cc4)n3)c2)cc1]

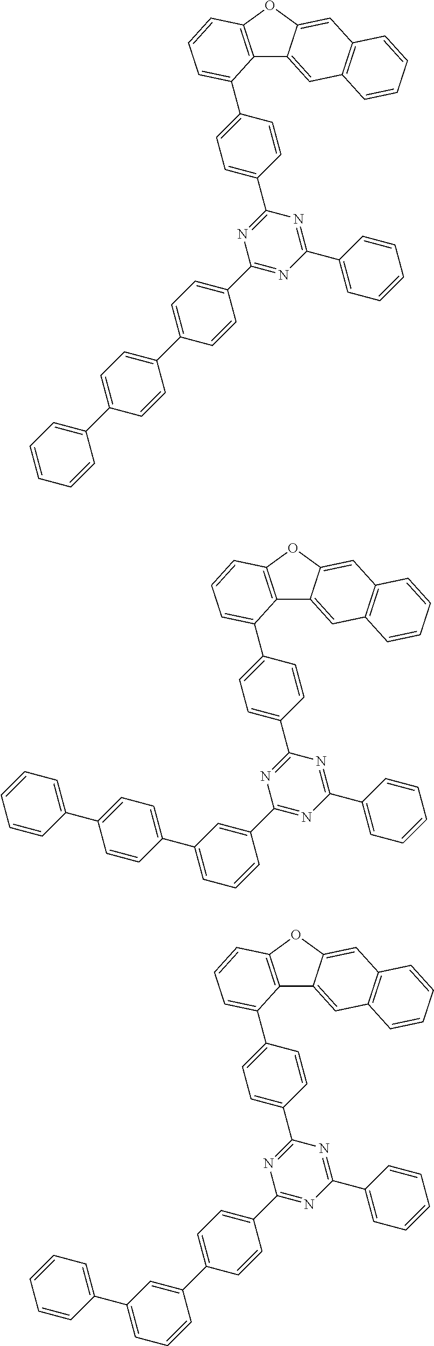 [SMILES: c1ccc(-c2ccc(-c3ccc(-c4nc(-c5ccccc5)nc(-c5ccc(-c6cccc7oc8cc9ccccc9cc8c67)cc5)n4)cc3)cc2)cc1 ; c1ccc(-c2ccc(-c3cccc(-c4nc(-c5ccccc5)nc(-c5ccc(-c6cccc7oc8cc9ccccc9cc8c67)cc5)n4)c3)cc2)cc1 ; c1ccc(-c2cccc(-c3ccc(-c4nc(-c5ccccc5)nc(-c5ccc(-c6cccc7oc8cc9ccccc9cc8c67)cc5)n4)cc3)c2)cc1]

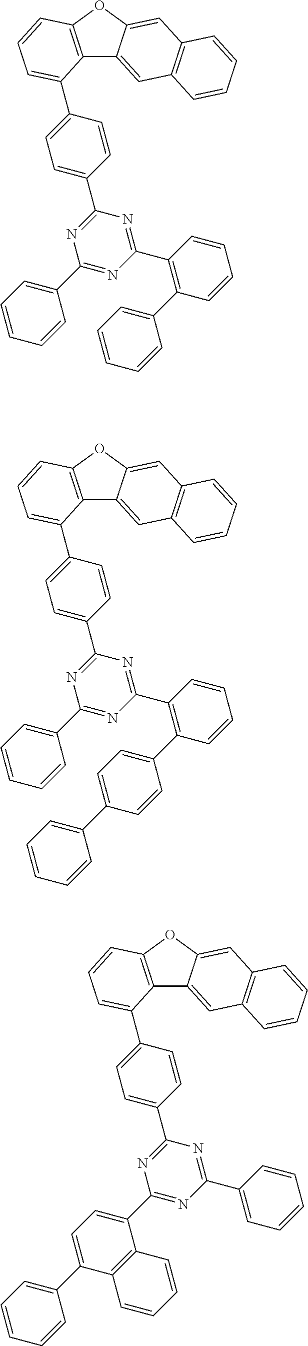 [SMILES: c1ccc(-c2nc(-c3ccc(-c4cccc5oc6cc7ccccc7cc6c45)cc3)nc(-c3ccccc3-c3ccccc3)n2)cc1 ; c1ccc(-c2ccc(-c3ccccc3-c3nc(-c4ccccc4)nc(-c4ccc(-c5cccc6oc7cc8ccccc8cc7c56)cc4)n3)cc2)cc1 ; c1ccc(-c2nc(-c3ccc(-c4cccc5oc6cc7ccccc7cc6c45)cc3)nc(-c3ccc(-c4ccccc4)c4ccccc34)n2)cc1]

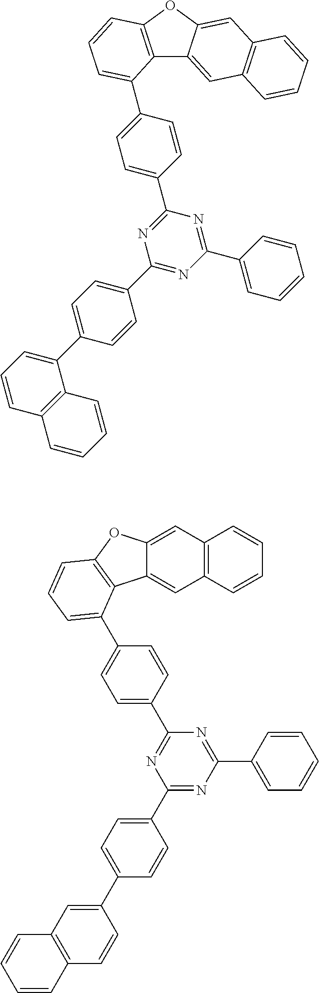 [SMILES: c1ccc(-c2nc(-c3ccc(-c4cccc5ccccc45)cc3)nc(-c3ccc(-c4cccc5oc6cc7ccccc7cc6c45)cc3)n2)cc1 ; c1ccc(-c2nc(-c3ccc(-c4ccc5ccccc5c4)cc3)nc(-c3ccc(-c4cccc5oc6cc7ccccc7cc6c45)cc3)n2)cc1]

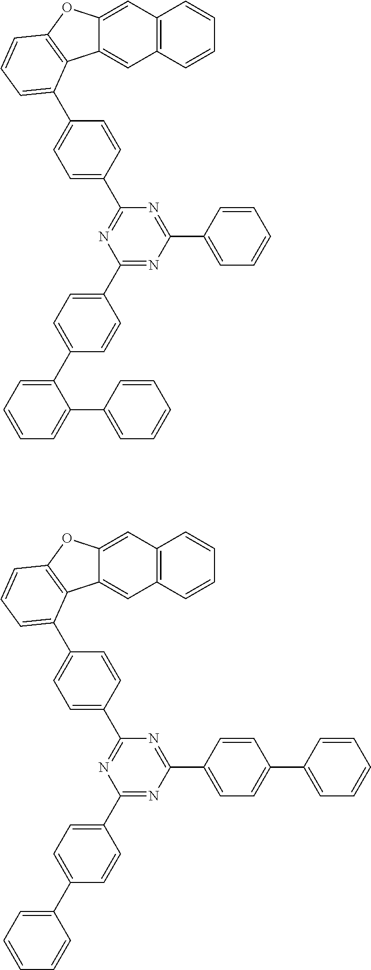 [SMILES: c1ccc(-c2nc(-c3ccc(-c4ccccc4-c4ccccc4)cc3)nc(-c3ccc(-c4cccc5oc6cc7ccccc7cc6c45)cc3)n2)cc1 ; c1ccc(-c2ccc(-c3nc(-c4ccc(-c5ccccc5)cc4)nc(-c4ccc(-c5cccc6oc7cc8ccccc8cc7c56)cc4)n3)cc2)cc1]

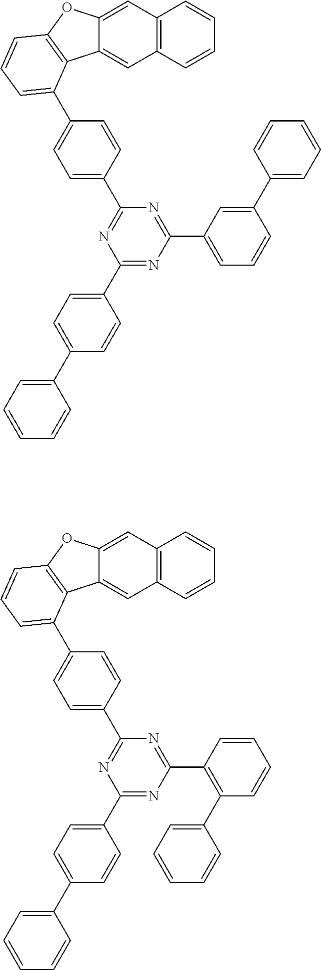 [SMILES: c1ccc(-c2ccc(-c3nc(-c4ccc(-c5cccc6oc7cc8ccccc8cc7c56)cc4)nc(-c4cccc(-c5ccccc5)c4)n3)cc2)cc1 ; c1ccc(-c2ccc(-c3nc(-c4ccc(-c5cccc6oc7cc8ccccc8cc7c56)cc4)nc(-c4ccccc4-c4ccccc4)n3)cc2)cc1]

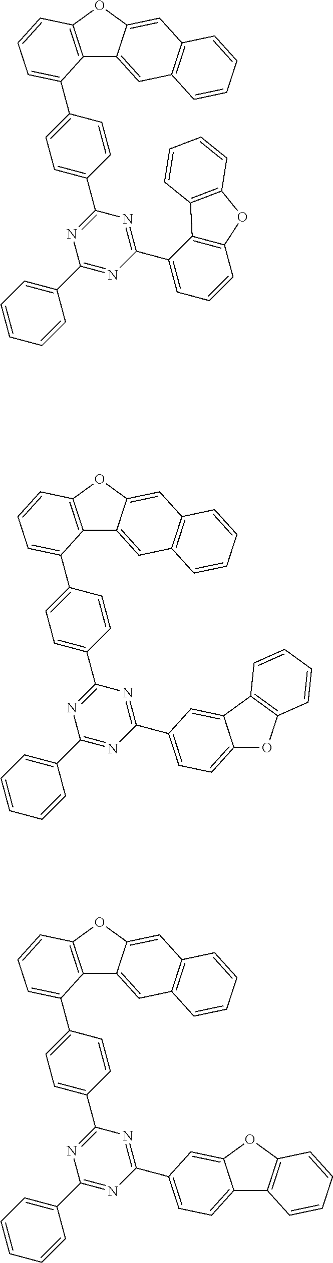 [SMILES: c1ccc(-c2nc(-c3ccc(-c4cccc5oc6cc7ccccc7cc6c45)cc3)nc(-c3cccc4oc5ccccc5c34)n2)cc1 ; c1ccc(-c2nc(-c3ccc(-c4cccc5oc6cc7ccccc7cc6c45)cc3)nc(-c3ccc4oc5ccccc5c4c3)n2)cc1 ; c1ccc(-c2nc(-c3ccc(-c4cccc5oc6cc7ccccc7cc6c45)cc3)nc(-c3ccc4c(c3)oc3ccccc34)n2)cc1]

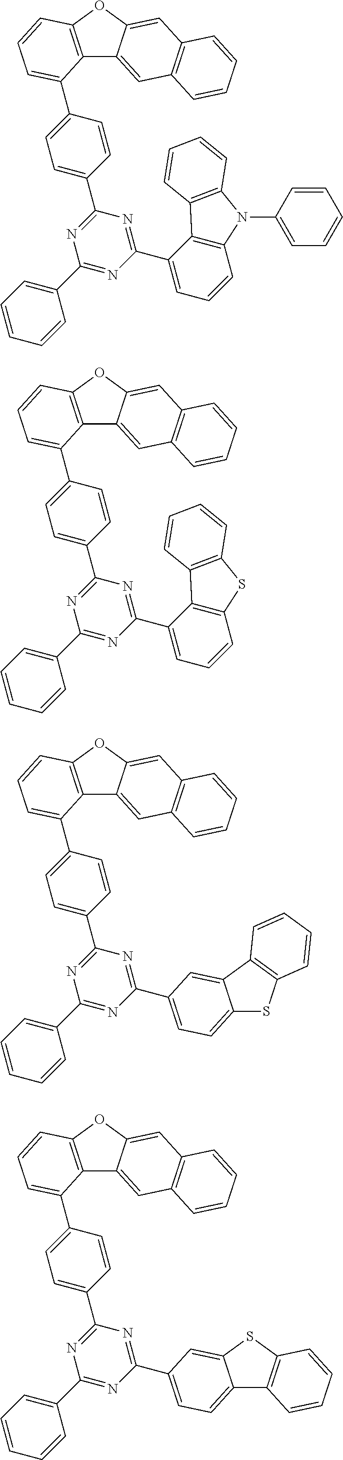

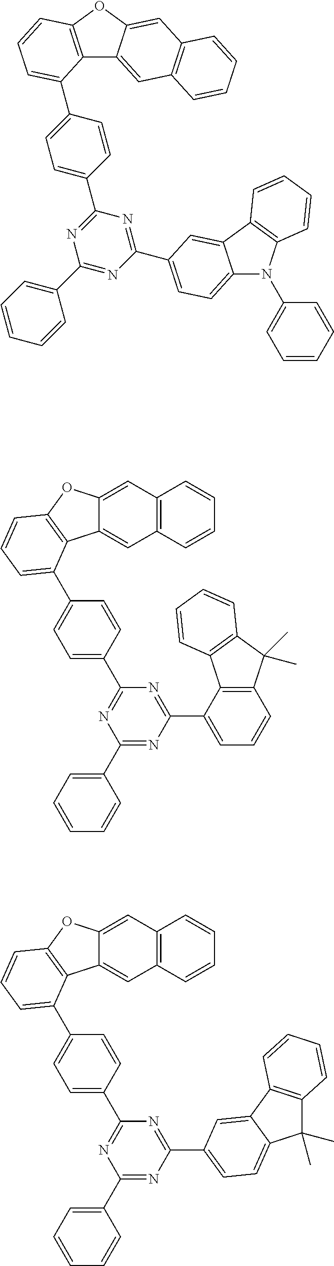 [SMILES: c1ccc(-c2nc(-c3ccc(-c4cccc5oc6cc7ccccc7cc6c45)cc3)nc(-c3ccc4c(c3)c3ccccc3n4-c3ccccc3)n2)cc1 ; CC1(C)c2ccccc2-c2c(-c3nc(-c4ccccc4)nc(-c4ccc(-c5cccc6oc7cc8ccccc8cc7c56)cc4)n3)cccc21 ; CC1(C)c2ccccc2-c2cc(-c3nc(-c4ccccc4)nc(-c4ccc(-c5cccc6oc7cc8ccccc8cc7c56)cc4)n3)ccc21]

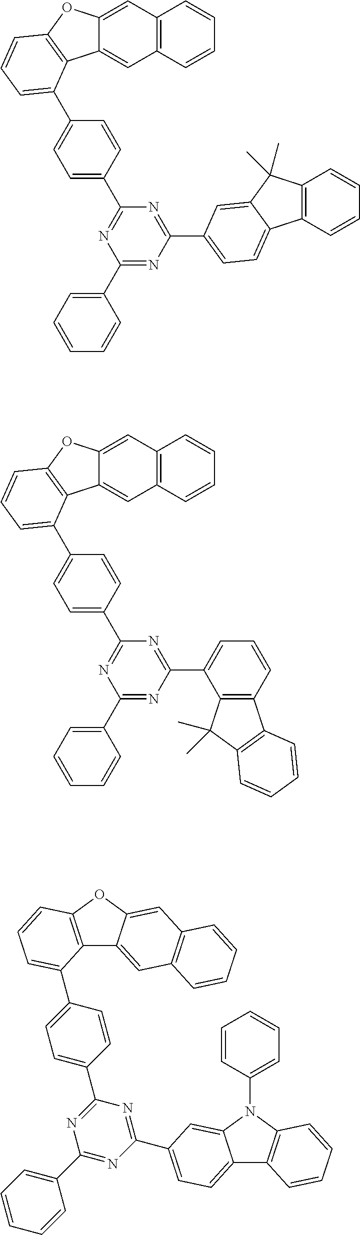 [SMILES: CC1(C)c2ccccc2-c2ccc(-c3nc(-c4ccccc4)nc(-c4ccc(-c5cccc6oc7cc8ccccc8cc7c56)cc4)n3)cc21 ; CC1(C)c2ccccc2-c2cccc(-c3nc(-c4ccccc4)nc(-c4ccc(-c5cccc6oc7cc8ccccc8cc7c56)cc4)n3)c21 ; c1ccc(-c2nc(-c3ccc(-c4cccc5oc6cc7ccccc7cc6c45)cc3)nc(-c3ccc4c5ccccc5n(-c5ccccc5)c4c3)n2)cc1]

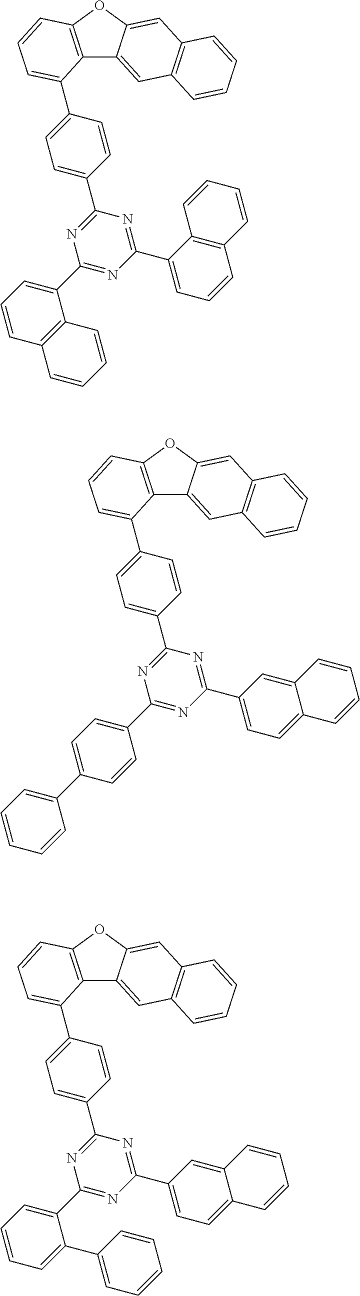 [SMILES: c1ccc2cc3c(cc2c1)oc1cccc(-c2ccc(-c4nc(-c5cccc6ccccc56)nc(-c5cccc6ccccc56)n4)cc2)c13 ; c1ccc(-c2ccc(-c3nc(-c4ccc(-c5cccc6oc7cc8ccccc8cc7c56)cc4)nc(-c4ccc5ccccc5c4)n3)cc2)cc1 ; c1ccc(-c2ccccc2-c2nc(-c3ccc(-c4cccc5oc6cc7ccccc7cc6c45)cc3)nc(-c3ccc4ccccc4c3)n2)cc1]

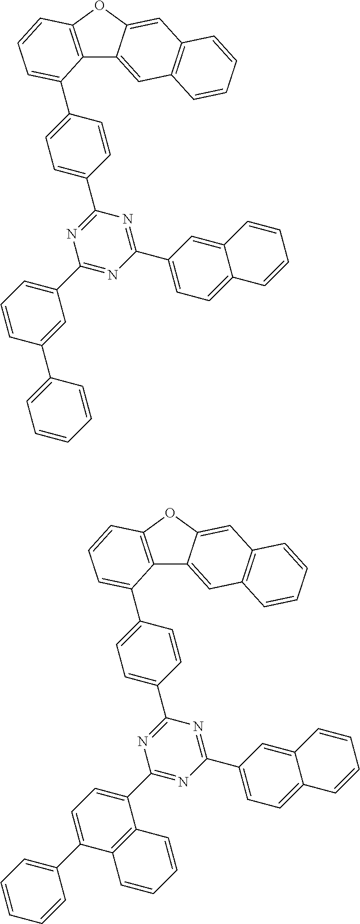 [SMILES: c1ccc(-c2cccc(-c3nc(-c4ccc(-c5cccc6oc7cc8ccccc8cc7c56)cc4)nc(-c4ccc5ccccc5c4)n3)c2)cc1 ; c1ccc(-c2ccc(-c3nc(-c4ccc(-c5cccc6oc7cc8ccccc8cc7c56)cc4)nc(-c4ccc5ccccc5c4)n3)c3ccccc23)cc1]

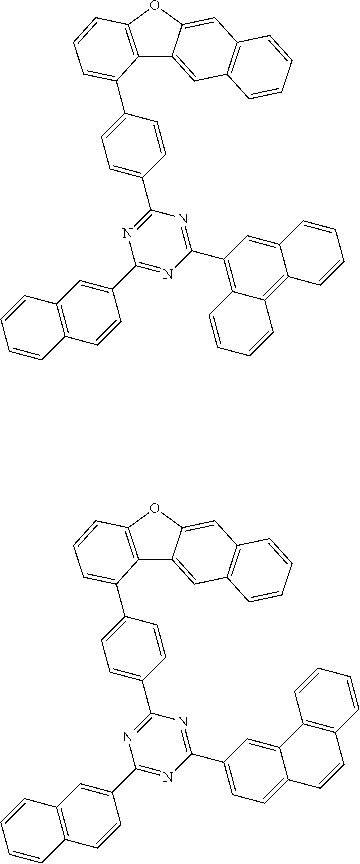 [SMILES: c1ccc2cc(-c3nc(-c4ccc(-c5cccc6oc7cc8ccccc8cc7c56)cc4)nc(-c4cc5ccccc5c5ccccc45)n3)ccc2c1 ; c1ccc2cc(-c3nc(-c4ccc(-c5cccc6oc7cc8ccccc8cc7c56)cc4)nc(-c4ccc5ccc6ccccc6c5c4)n3)ccc2c1]

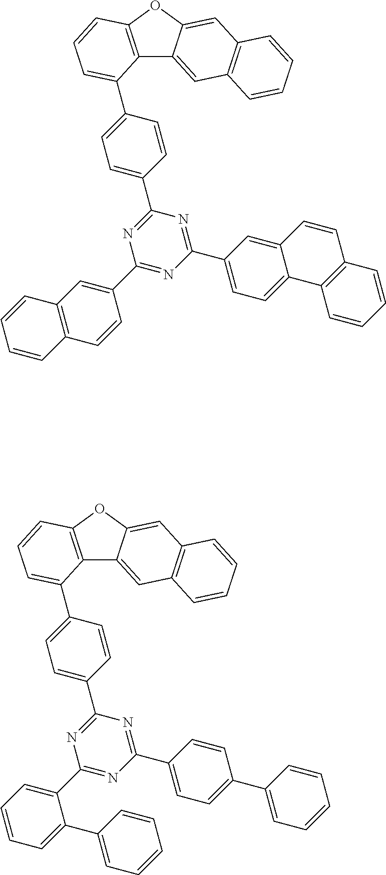 [SMILES: c1ccc2cc(-c3nc(-c4ccc(-c5cccc6oc7cc8ccccc8cc7c56)cc4)nc(-c4ccc5c(ccc6ccccc65)c4)n3)ccc2c1 ; c1ccc(-c2ccc(-c3nc(-c4ccc(-c5cccc6oc7cc8ccccc8cc7c56)cc4)nc(-c4ccccc4-c4ccccc4)n3)cc2)cc1]

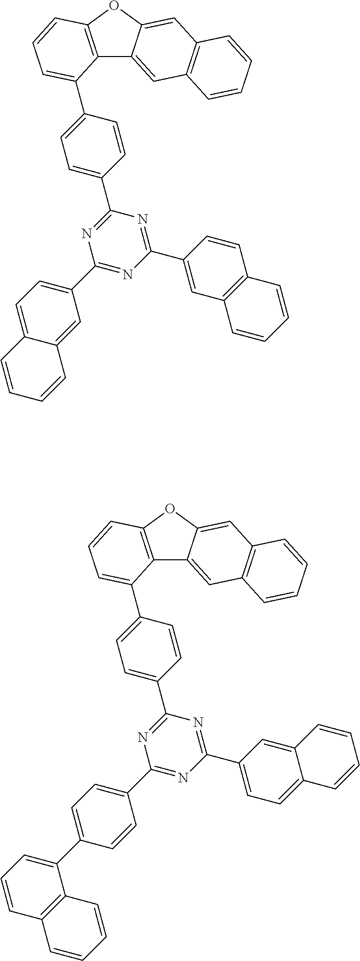 [SMILES: c1ccc2cc(-c3nc(-c4ccc(-c5cccc6oc7cc8ccccc8cc7c56)cc4)nc(-c4ccc5ccccc5c4)n3)ccc2c1 ; c1ccc2cc(-c3nc(-c4ccc(-c5cccc6ccccc56)cc4)nc(-c4ccc(-c5cccc6oc7cc8ccccc8cc7c56)cc4)n3)ccc2c1]

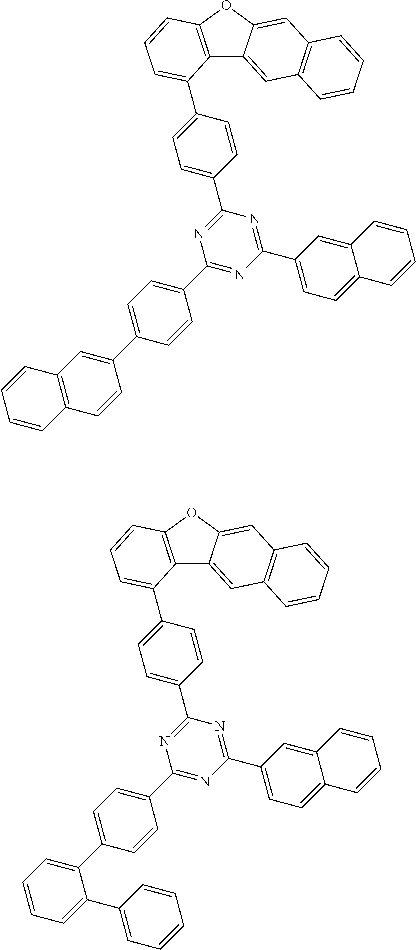 [SMILES: c1ccc2cc(-c3ccc(-c4nc(-c5ccc(-c6cccc7oc8cc9ccccc9cc8c67)cc5)nc(-c5ccc6ccccc6c5)n4)cc3)ccc2c1 ; c1ccc(-c2ccccc2-c2ccc(-c3nc(-c4ccc(-c5cccc6oc7cc8ccccc8cc7c56)cc4)nc(-c4ccc5ccccc5c4)n3)cc2)cc1]

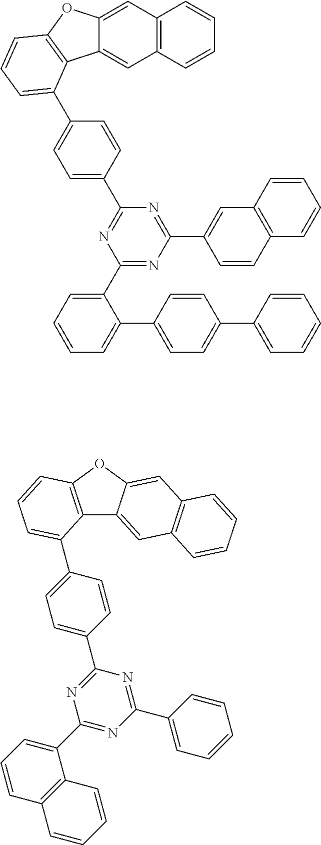 [SMILES: c1ccc(-c2ccc(-c3ccccc3-c3nc(-c4ccc(-c5cccc6oc7cc8ccccc8cc7c56)cc4)nc(-c4ccc5ccccc5c4)n3)cc2)cc1 ; c1ccc(-c2nc(-c3ccc(-c4cccc5oc6cc7ccccc7cc6c45)cc3)nc(-c3cccc4ccccc34)n2)cc1]

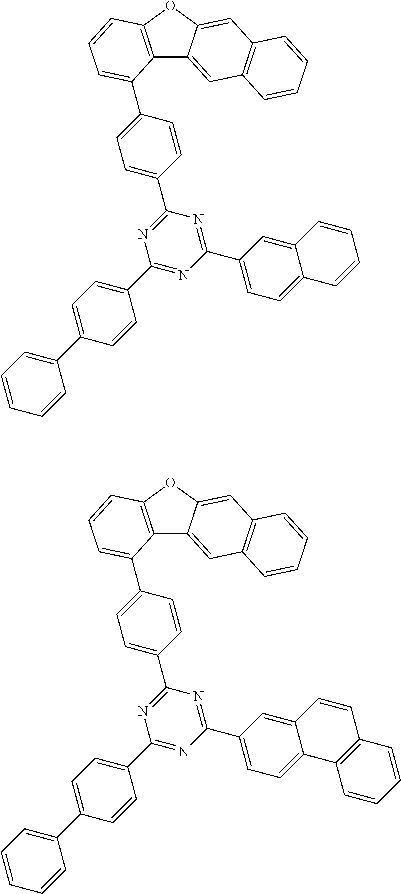 [SMILES: c1ccc(-c2ccc(-c3nc(-c4ccc(-c5cccc6oc7cc8ccccc8cc7c56)cc4)nc(-c4ccc5ccccc5c4)n3)cc2)cc1 ; c1ccc(-c2ccc(-c3nc(-c4ccc(-c5cccc6oc7cc8ccccc8cc7c56)cc4)nc(-c4ccc5c(ccc6ccccc65)c4)n3)cc2)cc1]

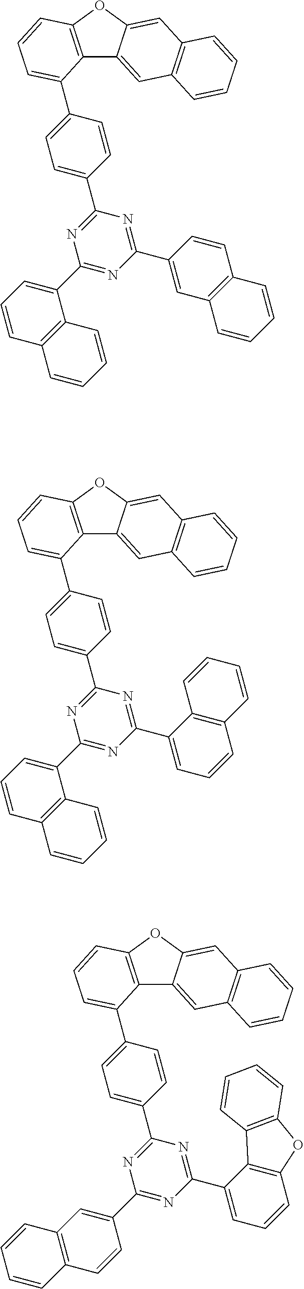 [SMILES: c1ccc2cc(-c3nc(-c4ccc(-c5cccc6oc7cc8ccccc8cc7c56)cc4)nc(-c4cccc5ccccc45)n3)ccc2c1 ; c1ccc2cc3c(cc2c1)oc1cccc(-c2ccc(-c4nc(-c5cccc6ccccc56)nc(-c5cccc6ccccc56)n4)cc2)c13 ; c1ccc2cc(-c3nc(-c4ccc(-c5cccc6oc7cc8ccccc8cc7c56)cc4)nc(-c4cccc5oc6ccccc6c45)n3)ccc2c1]

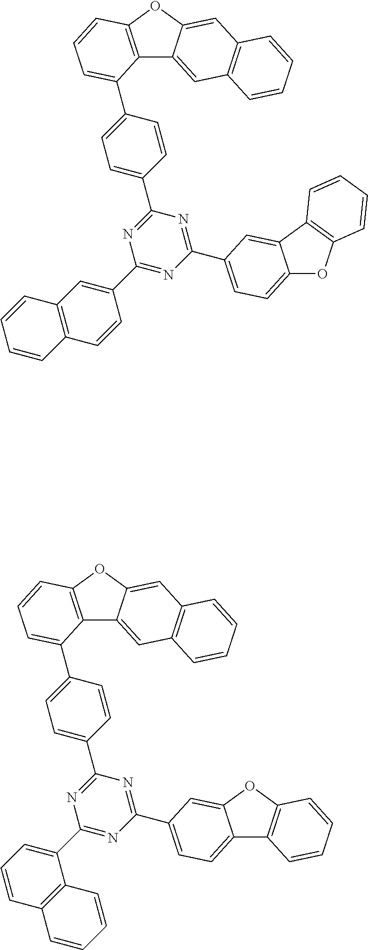 [SMILES: c1ccc2cc(-c3nc(-c4ccc(-c5cccc6oc7cc8ccccc8cc7c56)cc4)nc(-c4ccc5oc6ccccc6c5c4)n3)ccc2c1 ; c1ccc2cc3c(cc2c1)oc1cccc(-c2ccc(-c4nc(-c5ccc6c(c5)oc5ccccc56)nc(-c5cccc6ccccc56)n4)cc2)c13]

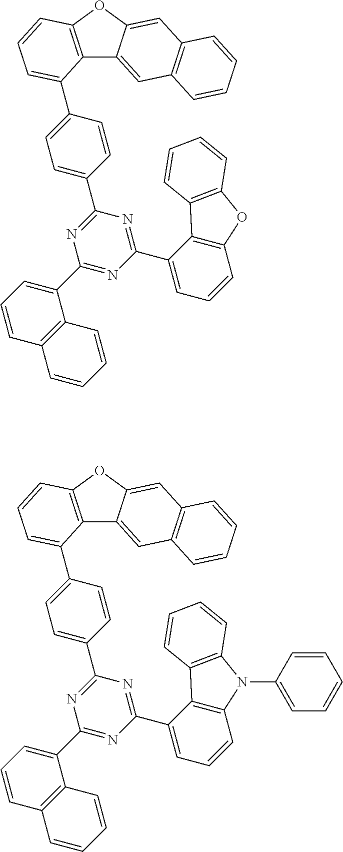 [SMILES: c1ccc2cc3c(cc2c1)oc1cccc(-c2ccc(-c4nc(-c5cccc6ccccc56)nc(-c5cccc6oc7ccccc7c56)n4)cc2)c13 ; c1ccc(-n2c3ccccc3c3c(-c4nc(-c5ccc(-c6cccc7oc8cc9ccccc9cc8c67)cc5)nc(-c5cccc6ccccc56)n4)cccc32)cc1]

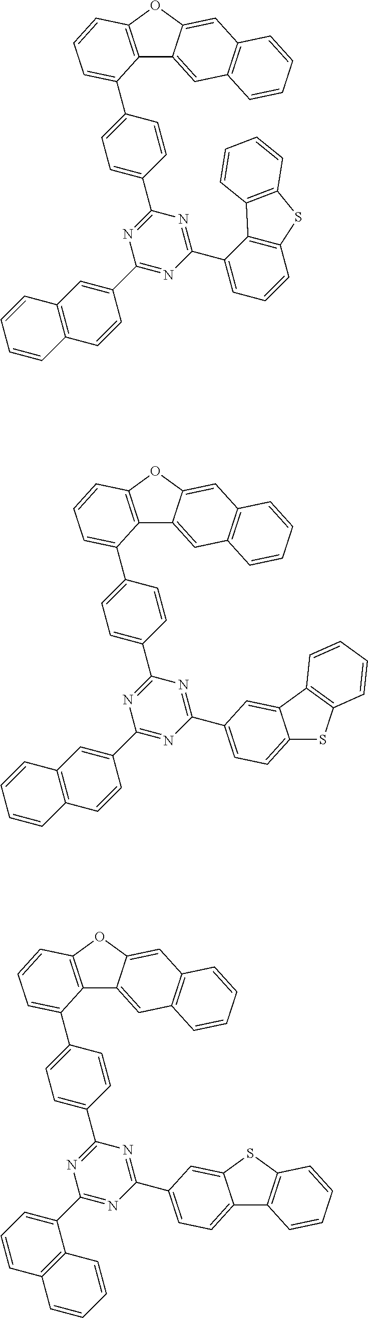 [SMILES: c1ccc2cc(-c3nc(-c4ccc(-c5cccc6oc7cc8ccccc8cc7c56)cc4)nc(-c4cccc5sc6ccccc6c45)n3)ccc2c1 ; c1ccc2cc(-c3nc(-c4ccc(-c5cccc6oc7cc8ccccc8cc7c56)cc4)nc(-c4ccc5sc6ccccc6c5c4)n3)ccc2c1 ; c1ccc2cc3c(cc2c1)oc1cccc(-c2ccc(-c4nc(-c5ccc6c(c5)sc5ccccc56)nc(-c5cccc6ccccc56)n4)cc2)c13]

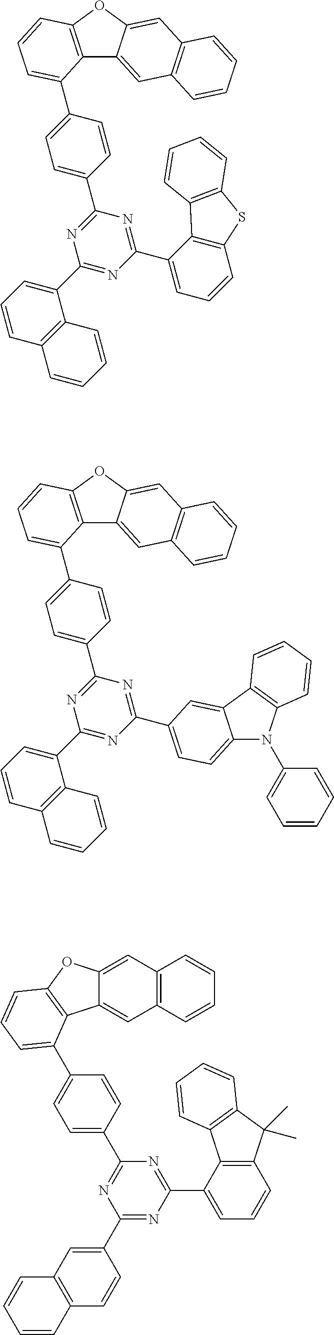 [SMILES: c1ccc2cc3c(cc2c1)oc1cccc(-c2ccc(-c4nc(-c5cccc6ccccc56)nc(-c5cccc6sc7ccccc7c56)n4)cc2)c13 ; c1ccc(-n2c3ccccc3c3cc(-c4nc(-c5ccc(-c6cccc7oc8cc9ccccc9cc8c67)cc5)nc(-c5cccc6ccccc56)n4)ccc32)cc1 ; CC1(C)c2ccccc2-c2c(-c3nc(-c4ccc(-c5cccc6oc7cc8ccccc8cc7c56)cc4)nc(-c4ccc5ccccc5c4)n3)cccc21]

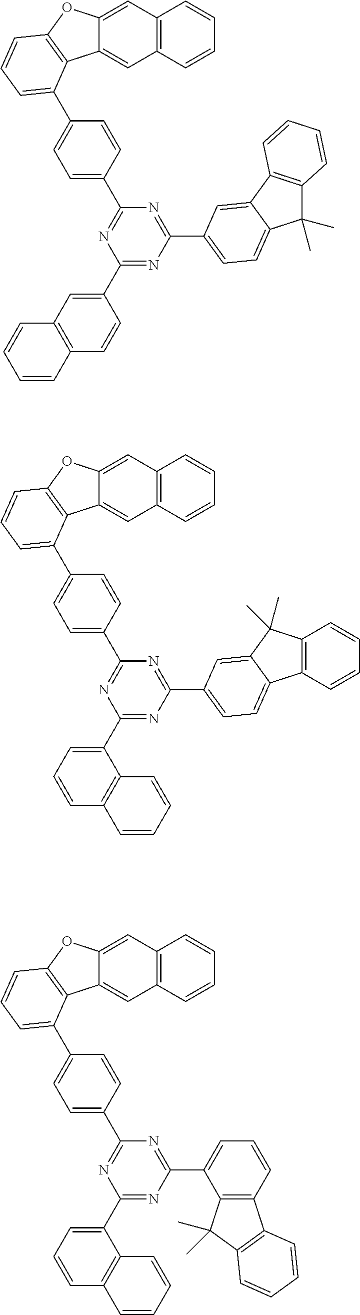 [SMILES: CC1(C)c2ccccc2-c2cc(-c3nc(-c4ccc(-c5cccc6oc7cc8ccccc8cc7c56)cc4)nc(-c4ccc5ccccc5c4)n3)ccc21 ; CC1(C)c2ccccc2-c2ccc(-c3nc(-c4ccc(-c5cccc6oc7cc8ccccc8cc7c56)cc4)nc(-c4cccc5ccccc45)n3)cc21 ; CC1(C)c2ccccc2-c2cccc(-c3nc(-c4ccc(-c5cccc6oc7cc8ccccc8cc7c56)cc4)nc(-c4cccc5ccccc45)n3)c21]

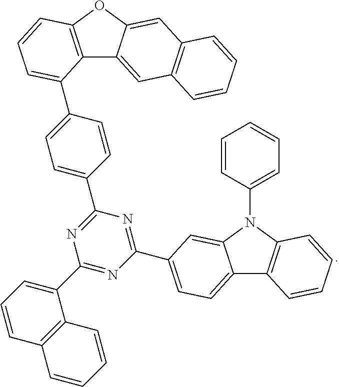 [SMILES: c1ccc(-n2c3ccccc3c3ccc(-c4nc(-c5ccc(-c6cccc7oc8cc9ccccc9cc8c67)cc5)nc(-c5cccc6ccccc56)n4)cc32)cc1]

8. The organic light emitting device according to claim 1,
wherein the compound of Chemical Formula 2 is represented by the following Chemical Formula 2-1: 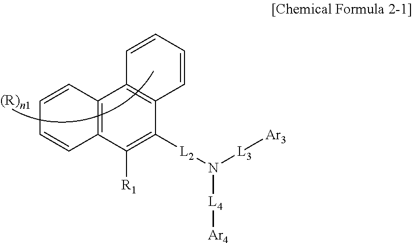
wherein in Chemical Formula 2-1,
R₁ is hydrogen, deuterium, or phenyl,
n1 is an integer of 0 to 8,
L₂, L₃, L₄, Ar₃, Ar₄ and R are the same as defined in claim 1.

9. The organic light emitting device according to claim 1,
wherein L₂ is phenylene; or phenylene substituted with at least one deuterium.

10. The organic light emitting device according to claim 1,
wherein L₃ and L₄ are each independently a single bond; phenylene; biphenyldiyl; or naphthylene, each of which except the single bond is
independently unsubstituted or substituted with at least one deuterium.

11. The organic light emitting device according to claim 1,
wherein Ar₃ and Ar₄ are each independently phenyl, biphenyl, terphenyl, naphthyl, phenanthrenyl, (phenyl)phenanthrenyl, triphenylenyl, phenylnaphthyl, naphthylphenyl, dimethylfluorenyl, diphenylfluorenyl, dibenzofuranyl, (phenyl)dibenzofuranyl, dibenzothiophenyl, (phenyl)dibenzothiophenyl, carbazole-9-yl, or 9-phenyl-9H-carbazolyl,
each of which is independently unsubstituted or substituted with at least one deuterium.

12. The organic light emitting device according to claim 1,
wherein the compound represented by Chemical Formula 2 is any one selected from the group consisting of the following: 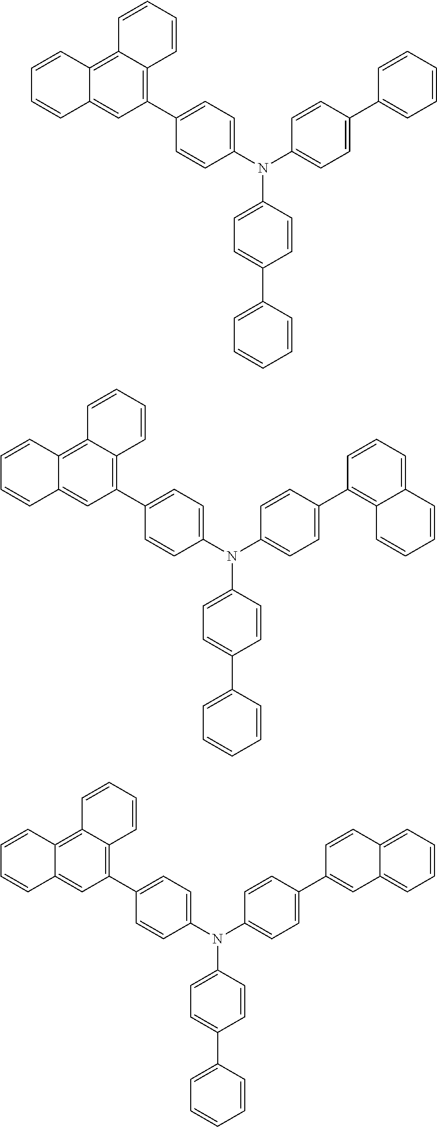 [SMILES: c1ccc(-c2ccc(N(c3ccc(-c4ccccc4)cc3)c3ccc(-c4cc5ccccc5c5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5ccccc5c4)cc3)c3ccc(-c4cc5ccccc5c5ccccc45)cc3)cc2)cc1] 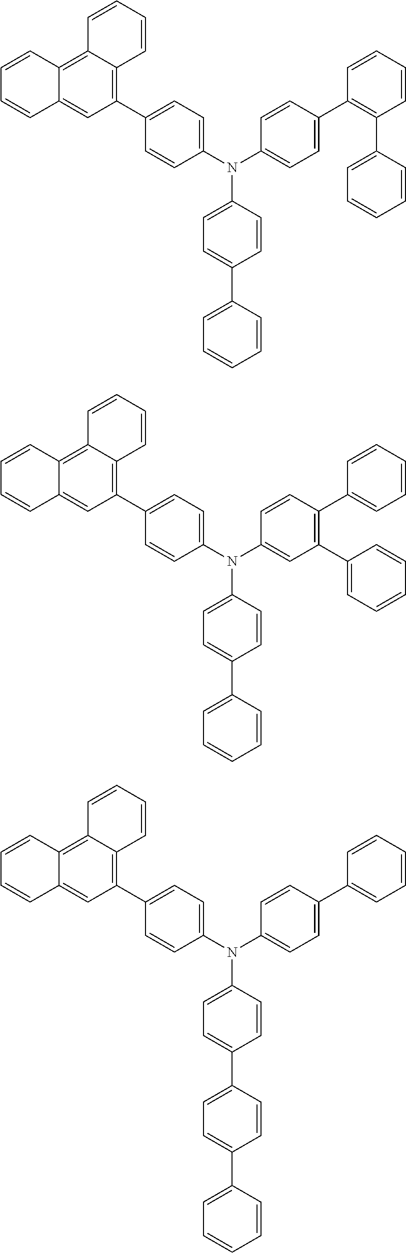 [SMILES: c1ccc(-c2ccc(N(c3ccc(-c4ccccc4-c4ccccc4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c(-c4ccccc4)c3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1] 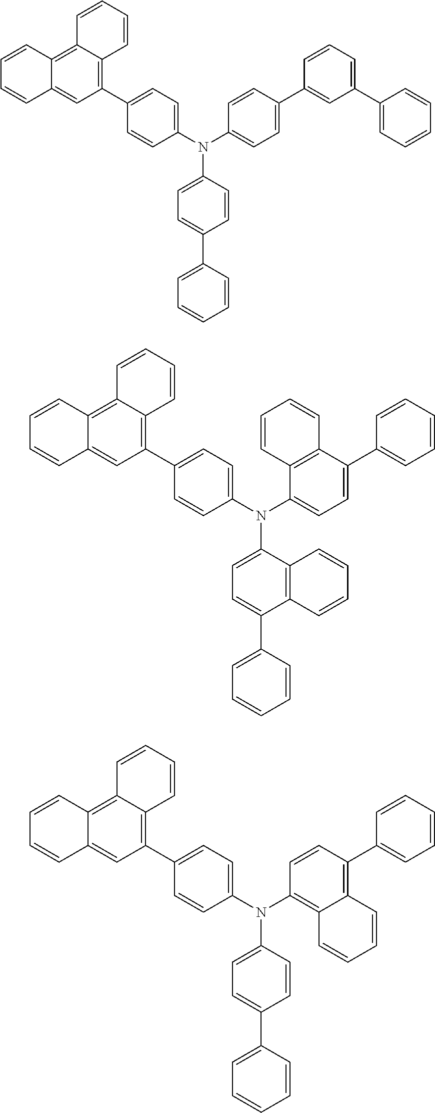 [SMILES: c1ccc(-c2ccc(N(c3ccc(-c4cccc(-c5ccccc5)c4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c4ccccc34)c3ccccc23)cc1 ; c1ccc(-c2ccc(N(c3ccc(-c4cc5ccccc5c5ccccc45)cc3)c3ccc(-c4ccccc4)c4ccccc34)cc2)cc1] 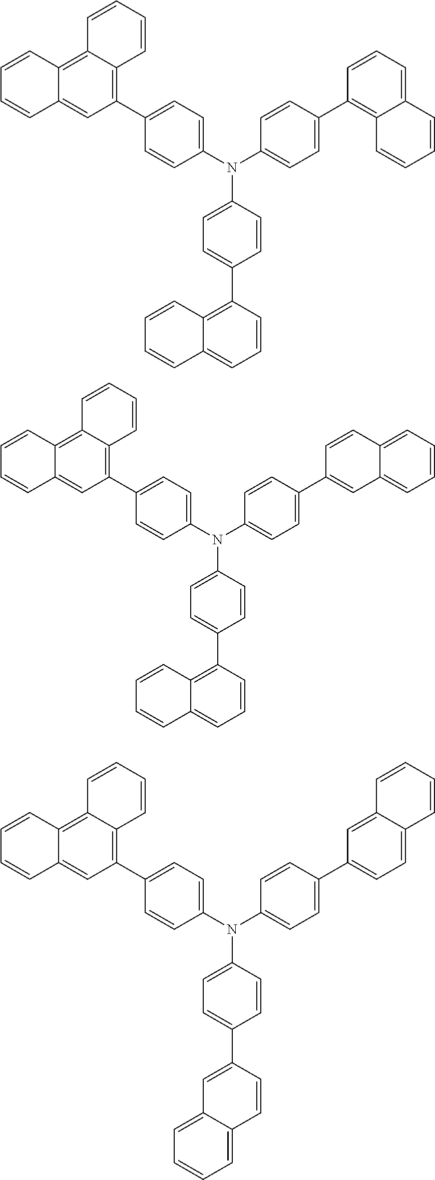 [SMILES: c1ccc2c(-c3ccc(N(c4ccc(-c5cccc6ccccc56)cc4)c4ccc(-c5cc6ccccc6c6ccccc56)cc4)cc3)cccc2c1 ; c1ccc2cc(-c3ccc(N(c4ccc(-c5cccc6ccccc56)cc4)c4ccc(-c5cc6ccccc6c6ccccc56)cc4)cc3)ccc2c1 ; c1ccc2cc(-c3ccc(N(c4ccc(-c5ccc6ccccc6c5)cc4)c4ccc(-c5cc6ccccc6c6ccccc56)cc4)cc3)ccc2c1] 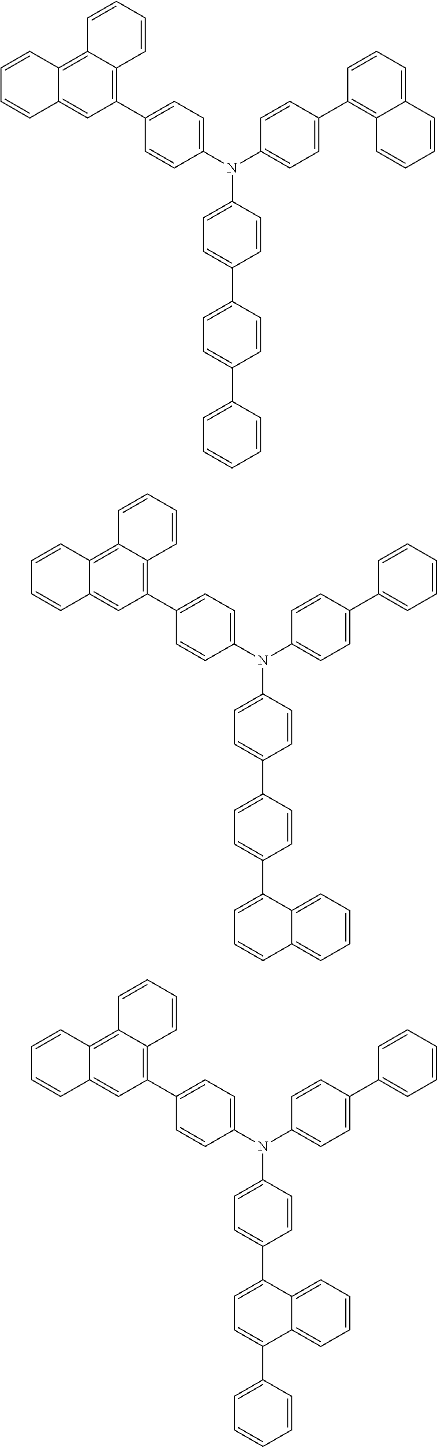 [SMILES: c1ccc(-c2ccc(-c3ccc(N(c4ccc(-c5cccc6ccccc56)cc4)c4ccc(-c5cc6ccccc6c6ccccc56)cc4)cc3)cc2)cc1 ; c1ccc(-c2ccc(N(c3ccc(-c4ccc(-c5cccc6ccccc56)cc4)cc3)c3ccc(-c4cc5ccccc5c5ccccc45)cc3)cc2)cc1 ; c1ccc(-c2ccc(N(c3ccc(-c4ccc(-c5ccccc5)c5ccccc45)cc3)c3ccc(-c4cc5ccccc5c5ccccc45)cc3)cc2)cc1] 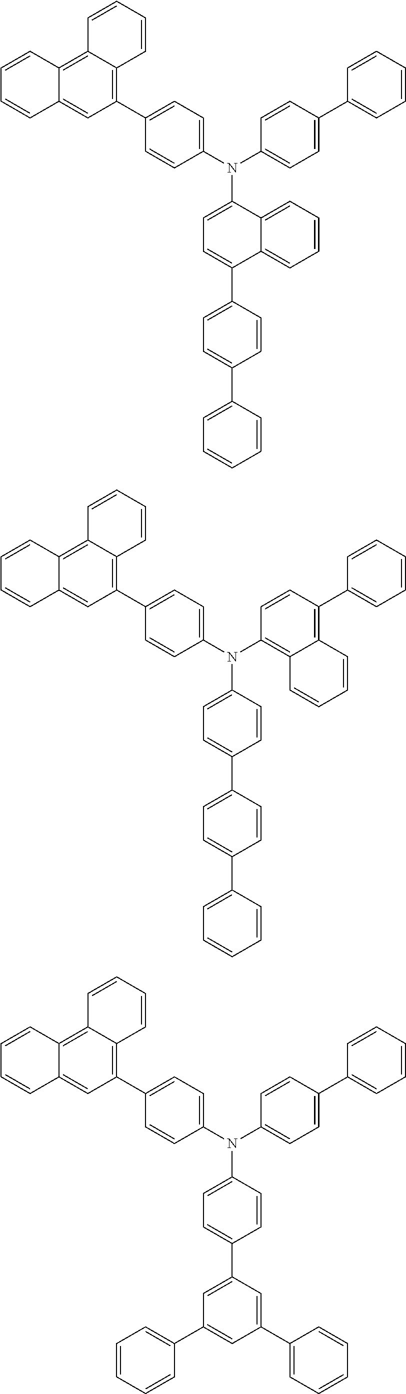 [SMILES: c1ccc(-c2ccc(-c3ccc(N(c4ccc(-c5ccccc5)cc4)c4ccc(-c5cc6ccccc6c6ccccc56)cc4)c4ccccc34)cc2)cc1 ; c1ccc(-c2ccc(-c3ccc(N(c4ccc(-c5cc6ccccc6c6ccccc56)cc4)c4ccc(-c5ccccc5)c5ccccc45)cc3)cc2)cc1 ; c1ccc(-c2ccc(N(c3ccc(-c4cc(-c5ccccc5)cc(-c5ccccc5)c4)cc3)c3ccc(-c4cc5ccccc5c5ccccc45)cc3)cc2)cc1] 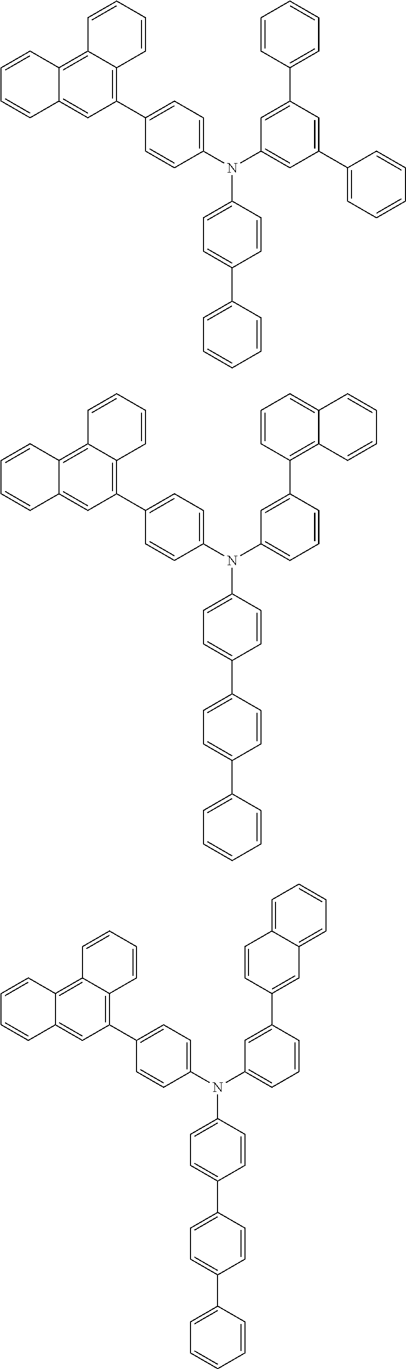 [SMILES: c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2ccc(-c3ccc(N(c4ccc(-c5cc6ccccc6c6ccccc56)cc4)c4cccc(-c5cccc6ccccc56)c4)cc3)cc2)cc1 ; c1ccc(-c2ccc(-c3ccc(N(c4ccc(-c5cc6ccccc6c6ccccc56)cc4)c4cccc(-c5ccc6ccccc6c5)c4)cc3)cc2)cc1] 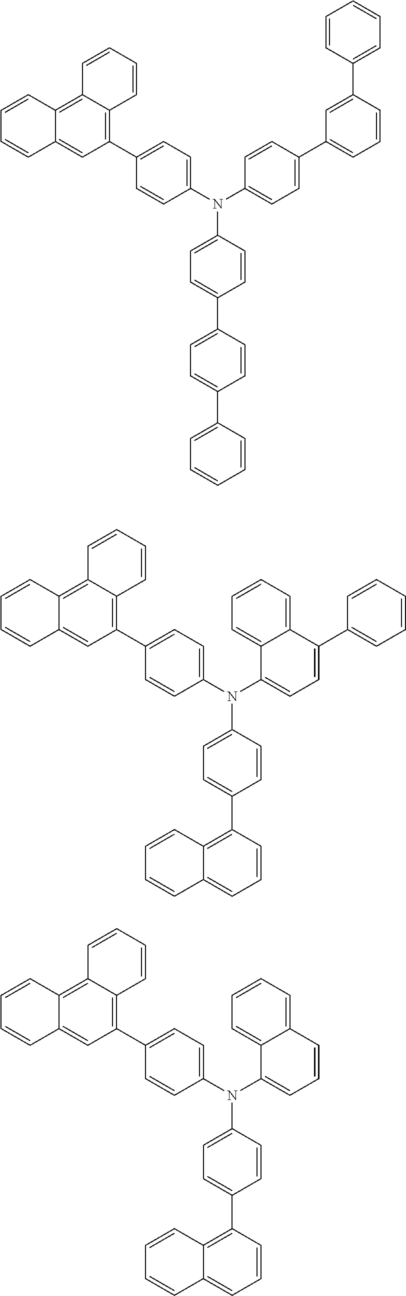 [SMILES: c1ccc(-c2ccc(-c3ccc(N(c4ccc(-c5cccc(-c6ccccc6)c5)cc4)c4ccc(-c5cc6ccccc6c6ccccc56)cc4)cc3)cc2)cc1 ; c1ccc(-c2ccc(N(c3ccc(-c4cccc5ccccc45)cc3)c3ccc(-c4cc5ccccc5c5ccccc45)cc3)c3ccccc23)cc1 ; c1ccc2c(-c3ccc(N(c4ccc(-c5cc6ccccc6c6ccccc56)cc4)c4cccc5ccccc45)cc3)cccc2c1] 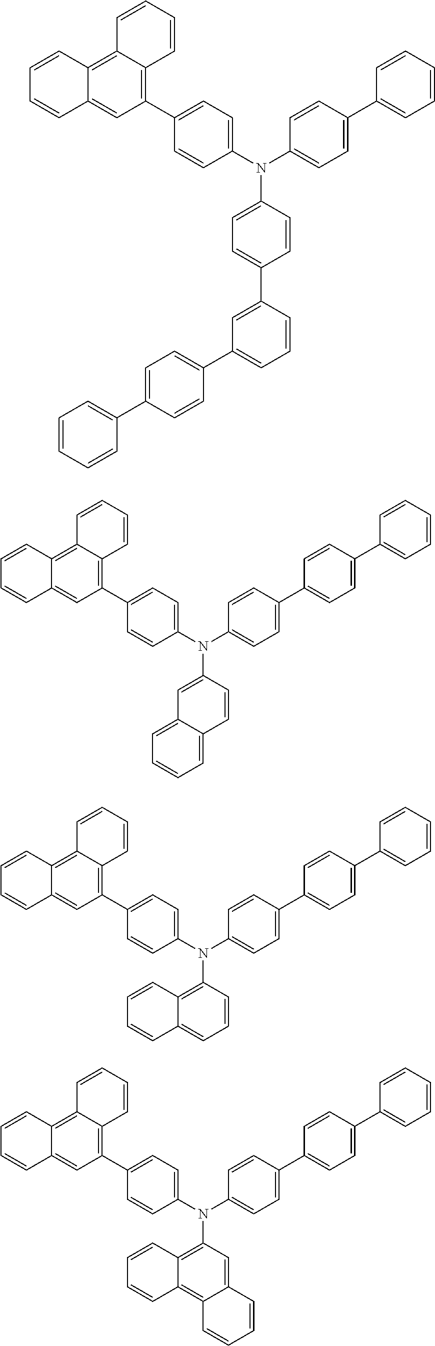 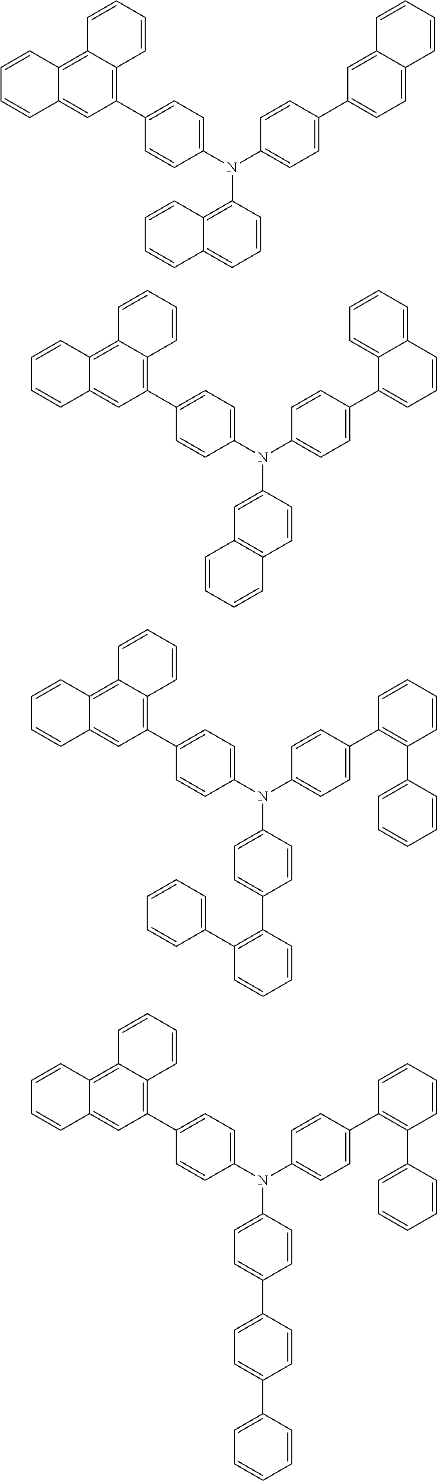 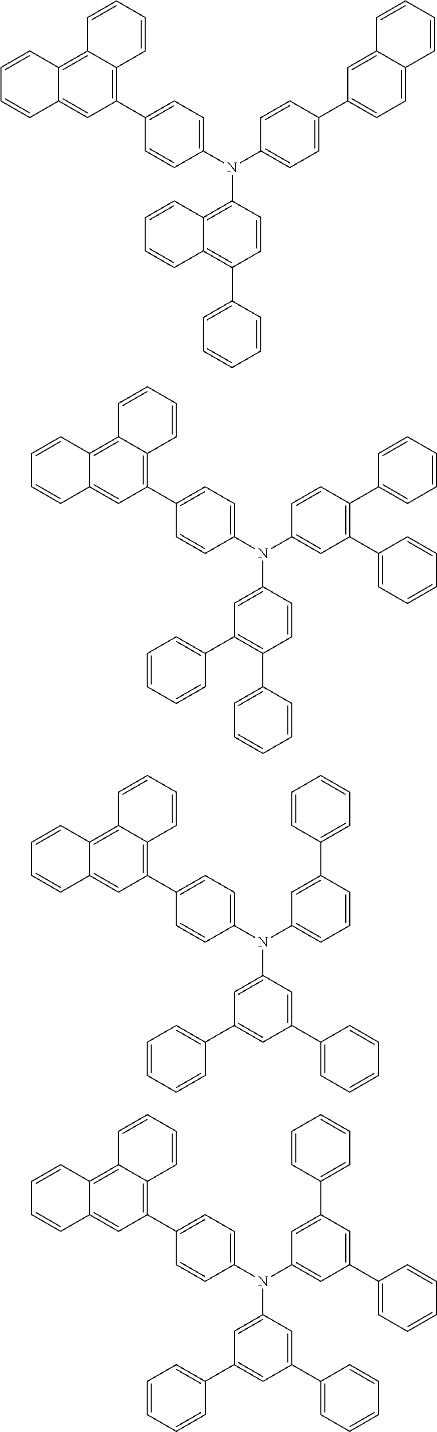 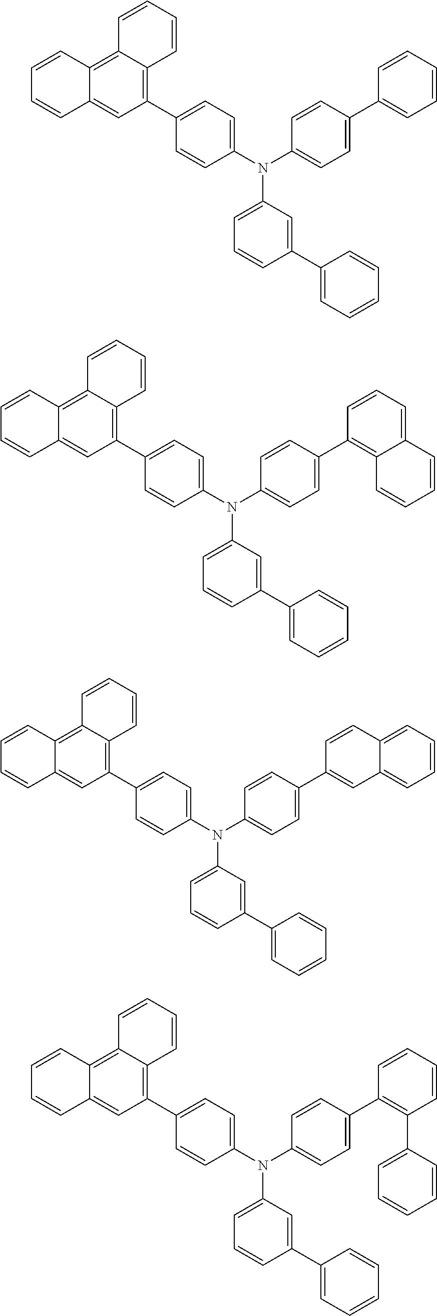 [SMILES: c1ccc(-c2ccc(N(c3ccc(-c4cc5ccccc5c5ccccc45)cc3)c3cccc(-c4ccccc4)c3)cc2)cc1 ; c1ccc(-c2cccc(N(c3ccc(-c4cccc5ccccc45)cc3)c3ccc(-c4cc5ccccc5c5ccccc45)cc3)c2)cc1 ; c1ccc(-c2cccc(N(c3ccc(-c4ccc5ccccc5c4)cc3)c3ccc(-c4cc5ccccc5c5ccccc45)cc3)c2)cc1 ; c1ccc(-c2cccc(N(c3ccc(-c4ccccc4-c4ccccc4)cc3)c3ccc(-c4cc5ccccc5c5ccccc45)cc3)c2)cc1] 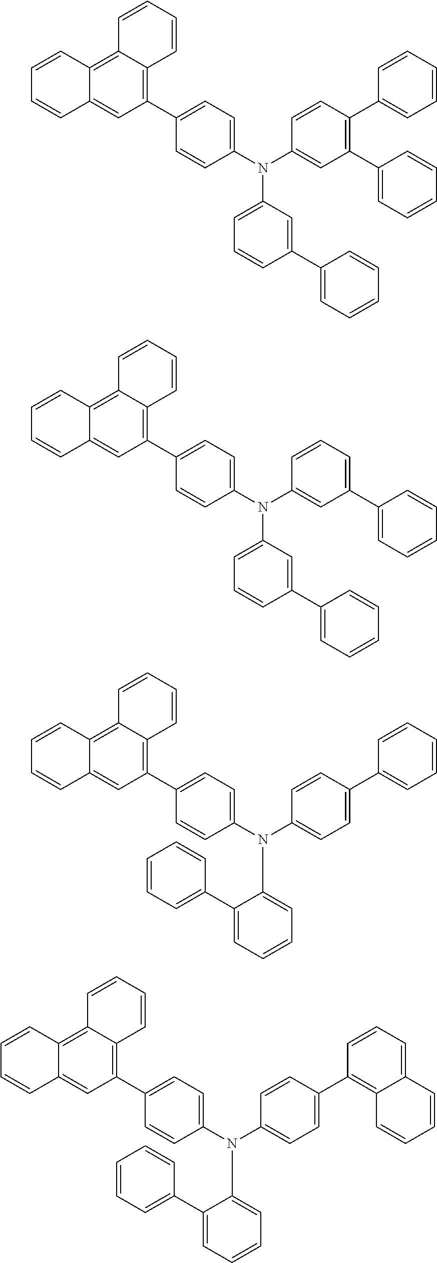 [SMILES: c1ccc(-c2cccc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cccc(-c4ccccc4)c3)c2)cc1 ; c1ccc(-c2ccc(N(c3ccc(-c4cc5ccccc5c5ccccc45)cc3)c3ccccc3-c3ccccc3)cc2)cc1 ; c1ccc(-c2ccccc2N(c2ccc(-c3cccc4ccccc34)cc2)c2ccc(-c3cc4ccccc4c4ccccc34)cc2)cc1] 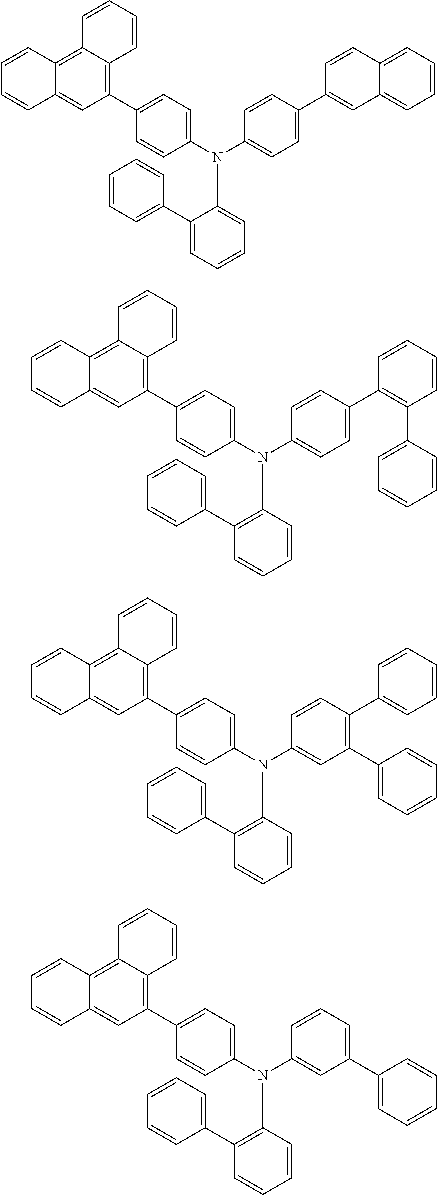 [SMILES: c1ccc(-c2ccccc2N(c2ccc(-c3ccc4ccccc4c3)cc2)c2ccc(-c3cc4ccccc4c4ccccc34)cc2)cc1 ; c1ccc(-c2ccccc2-c2ccc(N(c3ccc(-c4cc5ccccc5c5ccccc45)cc3)c3ccccc3-c3ccccc3)cc2)cc1 ; c1ccc(-c2ccc(N(c3ccc(-c4cc5ccccc5c5ccccc45)cc3)c3ccccc3-c3ccccc3)cc2-c2ccccc2)cc1 ; c1ccc(-c2cccc(N(c3ccc(-c4cc5ccccc5c5ccccc45)cc3)c3ccccc3-c3ccccc3)c2)cc1] 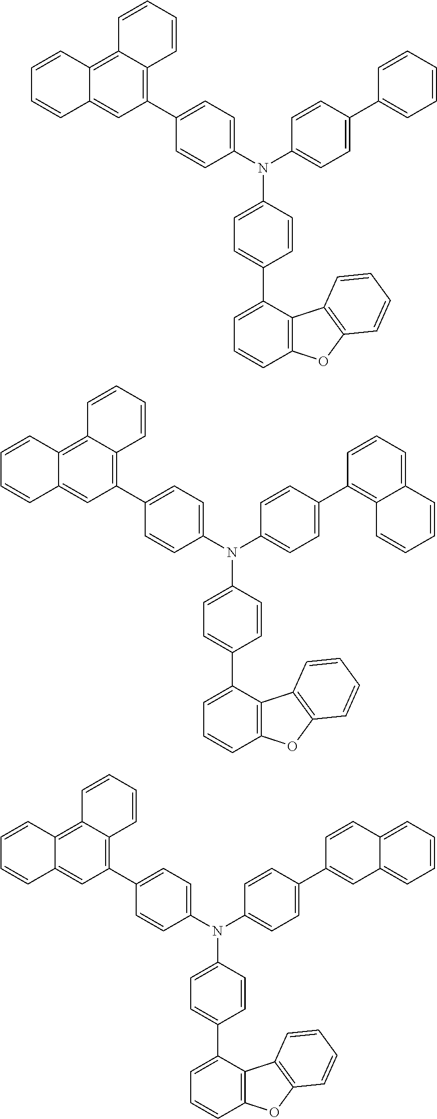 [SMILES: c1ccc(-c2ccc(N(c3ccc(-c4cc5ccccc5c5ccccc45)cc3)c3ccc(-c4cccc5oc6ccccc6c45)cc3)cc2)cc1 ; c1ccc2c(-c3ccc(N(c4ccc(-c5cc6ccccc6c6ccccc56)cc4)c4ccc(-c5cccc6oc7ccccc7c56)cc4)cc3)cccc2c1 ; c1ccc2cc(-c3ccc(N(c4ccc(-c5cc6ccccc6c6ccccc56)cc4)c4ccc(-c5cccc6oc7ccccc7c56)cc4)cc3)ccc2c1] 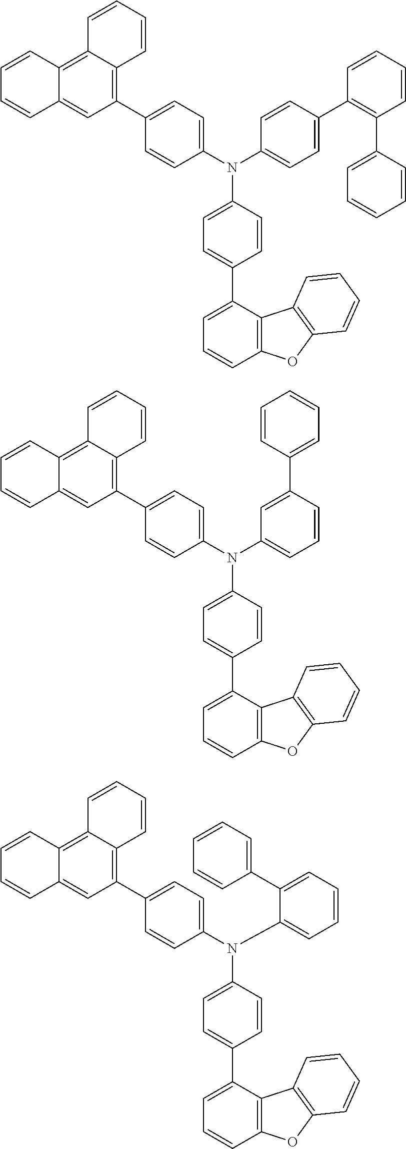 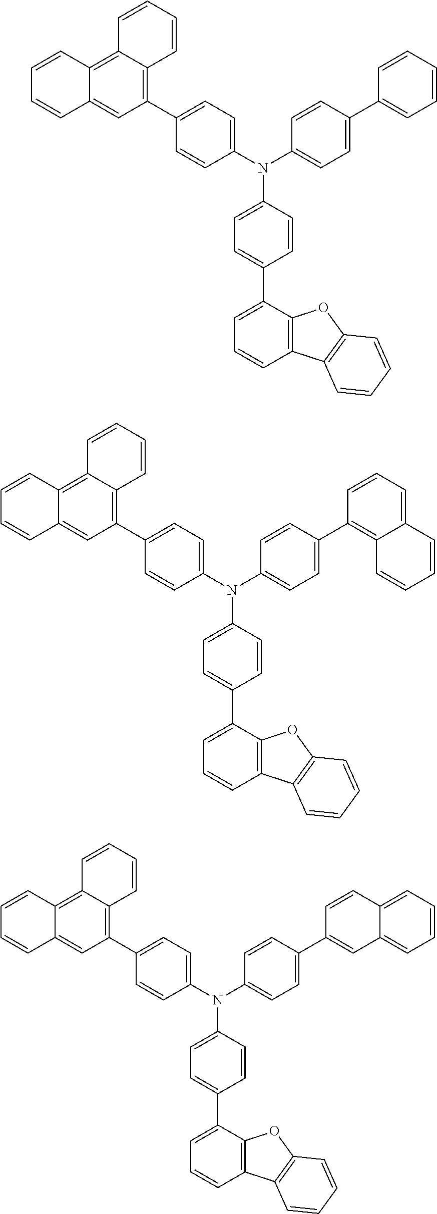 [SMILES: c1ccc(-c2ccc(N(c3ccc(-c4cc5ccccc5c5ccccc45)cc3)c3ccc(-c4cccc5c4oc4ccccc45)cc3)cc2)cc1 ; c1ccc2c(-c3ccc(N(c4ccc(-c5cc6ccccc6c6ccccc56)cc4)c4ccc(-c5cccc6c5oc5ccccc56)cc4)cc3)cccc2c1 ; c1ccc2cc(-c3ccc(N(c4ccc(-c5cc6ccccc6c6ccccc56)cc4)c4ccc(-c5cccc6c5oc5ccccc56)cc4)cc3)ccc2c1] 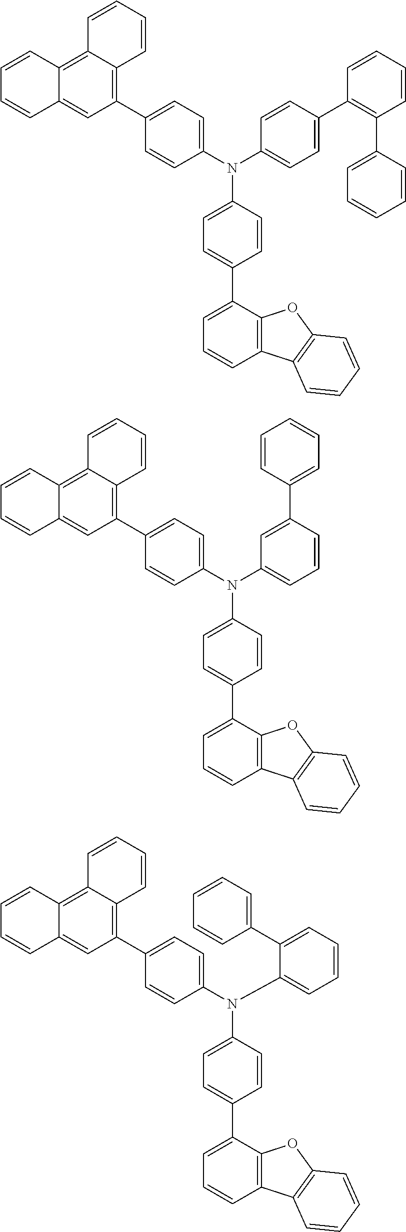 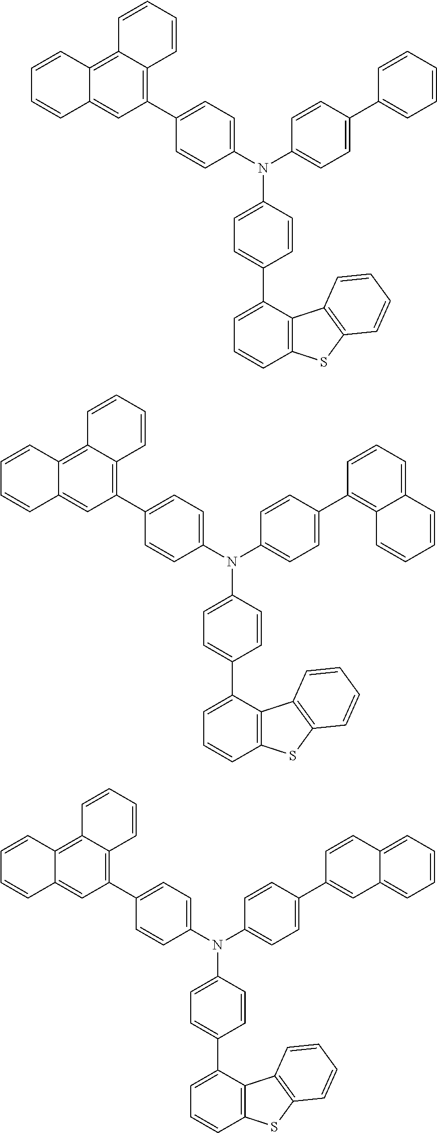 [SMILES: c1ccc(-c2ccc(N(c3ccc(-c4cc5ccccc5c5ccccc45)cc3)c3ccc(-c4cccc5sc6ccccc6c45)cc3)cc2)cc1 ; c1ccc2c(-c3ccc(N(c4ccc(-c5cc6ccccc6c6ccccc56)cc4)c4ccc(-c5cccc6sc7ccccc7c56)cc4)cc3)cccc2c1 ; c1ccc2cc(-c3ccc(N(c4ccc(-c5cc6ccccc6c6ccccc56)cc4)c4ccc(-c5cccc6sc7ccccc7c56)cc4)cc3)ccc2c1] 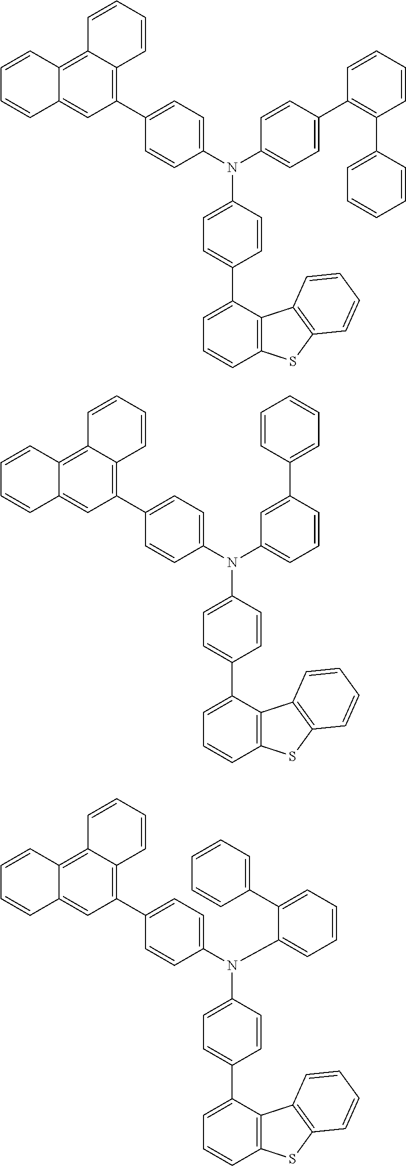 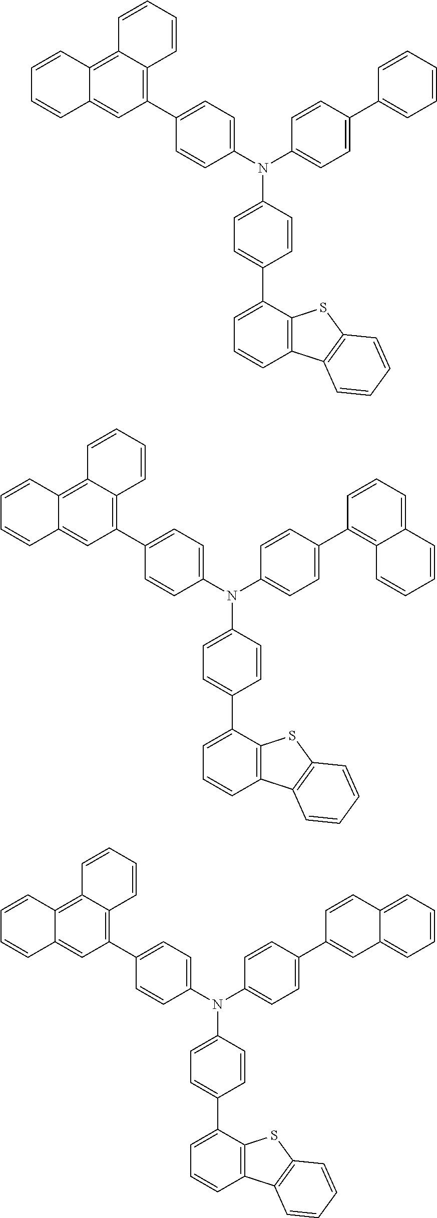 [SMILES: c1ccc(-c2ccc(N(c3ccc(-c4cc5ccccc5c5ccccc45)cc3)c3ccc(-c4cccc5c4sc4ccccc45)cc3)cc2)cc1 ; c1ccc2c(-c3ccc(N(c4ccc(-c5cc6ccccc6c6ccccc56)cc4)c4ccc(-c5cccc6c5sc5ccccc56)cc4)cc3)cccc2c1 ; c1ccc2cc(-c3ccc(N(c4ccc(-c5cc6ccccc6c6ccccc56)cc4)c4ccc(-c5cccc6c5sc5ccccc56)cc4)cc3)ccc2c1] 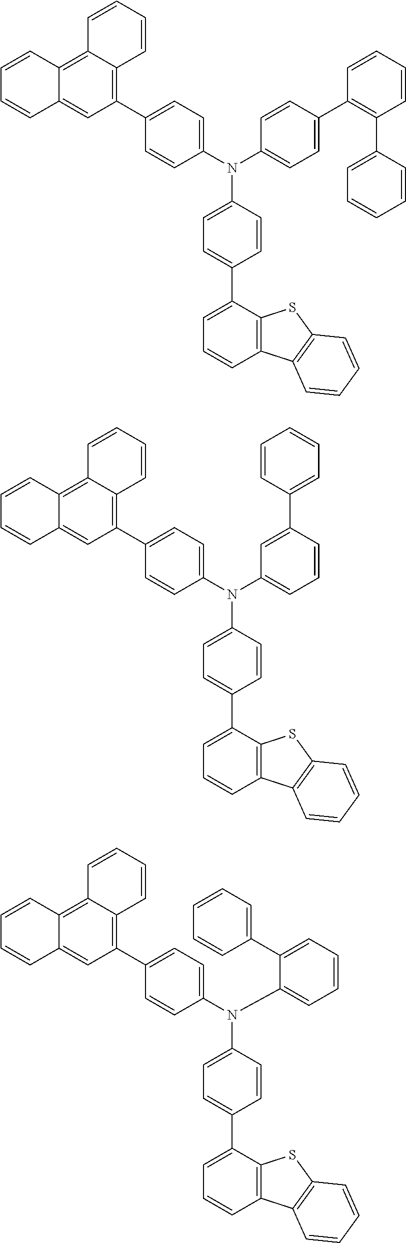 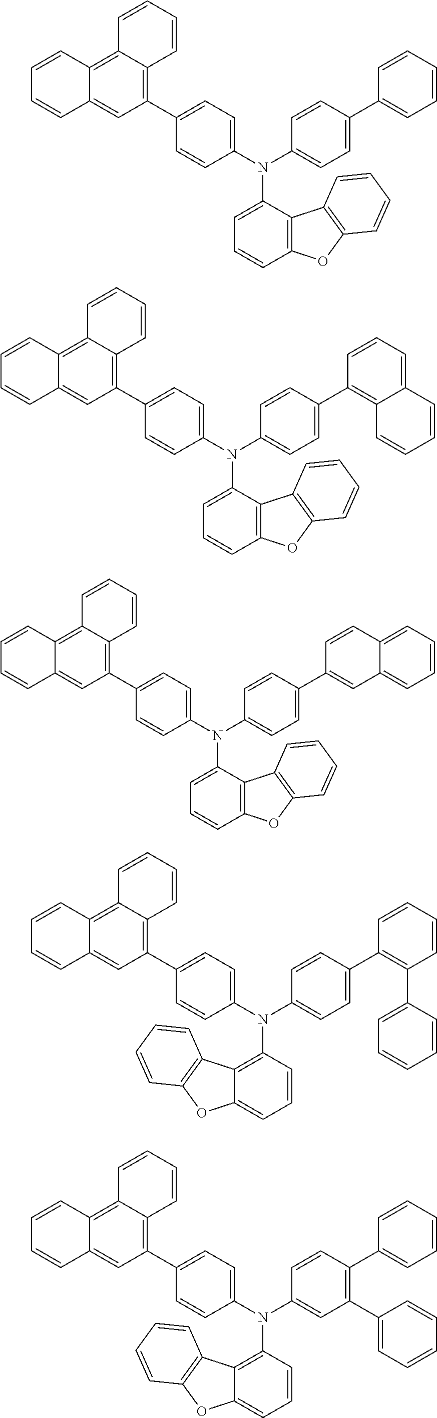 [SMILES: c1ccc(-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)cc3)cccc2c1 ; c1ccc2cc(-c3ccc(N(c4ccc(-c5cc6ccccc6c6ccccc56)cc4)c4cccc5oc6ccccc6c45)cc3)ccc2c1 ; c1ccc(-c2ccccc2-c2ccc(N(c3ccc(-c4cc5ccccc5c5ccccc45)cc3)c3cccc4oc5ccccc5c34)cc2)cc1 ; c1ccc(-c2ccc(N(c3ccc(-c4cc5ccccc5c5ccccc45)cc3)c3cccc4oc5ccccc5c34)cc2-c2ccccc2)cc1] 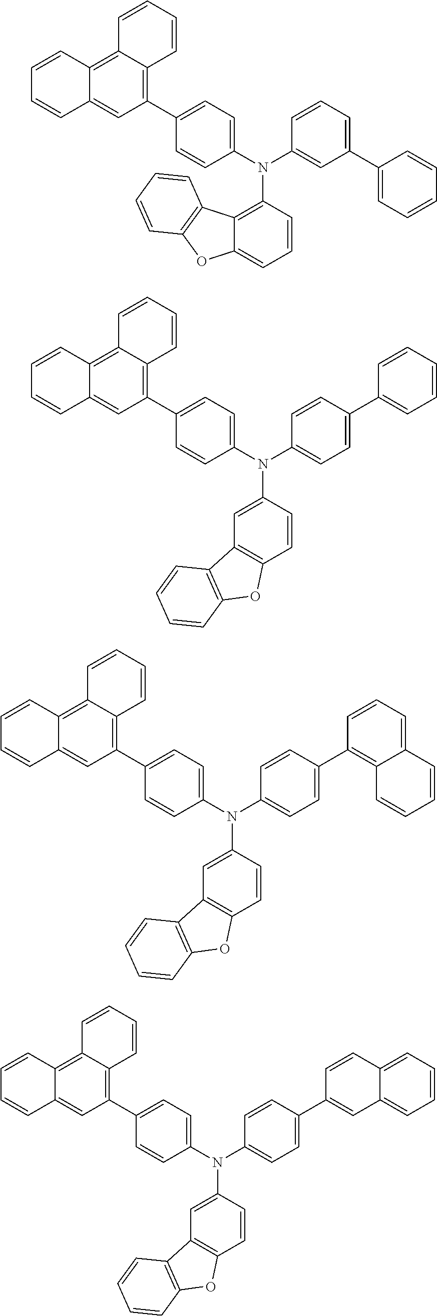 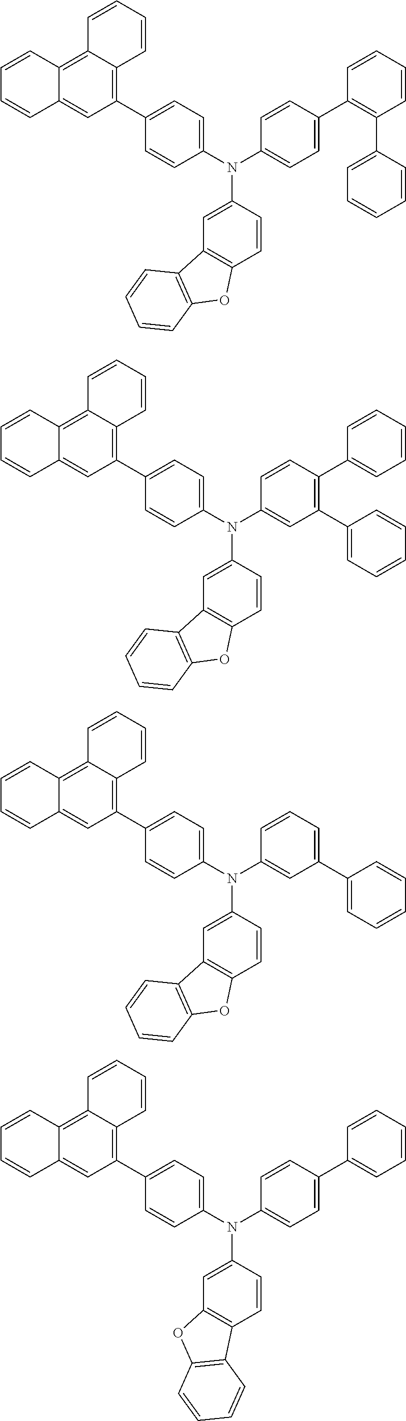 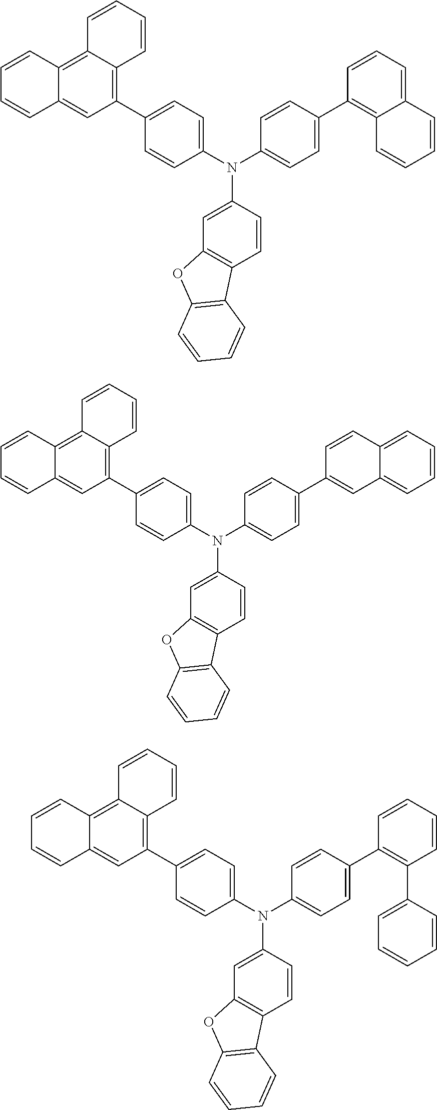 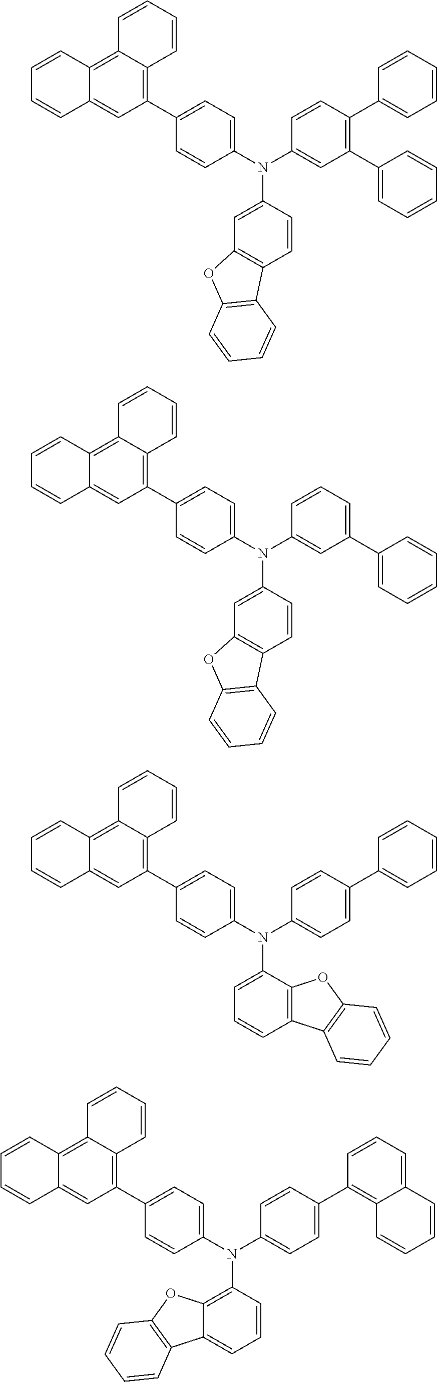 [SMILES: c1ccc(-c2ccc(N(c3ccc(-c4cc5ccccc5c5ccccc45)cc3)c3ccc4c(c3)oc3ccccc34)cc2-c2ccccc2)cc1 ; c1ccc(-c2cccc(N(c3ccc(-c4cc5ccccc5c5ccccc45)cc3)c3ccc4c(c3)oc3ccccc34)c2)cc1 ; c1ccc(-c2ccc(N(c3ccc(-c4cc5ccccc5c5ccccc45)cc3)c3cccc4c3oc3ccccc34)cc2)cc1 ; c1ccc2c(-c3ccc(N(c4ccc(-c5cc6ccccc6c6ccccc56)cc4)c4cccc5c4oc4ccccc45)cc3)cccc2c1] 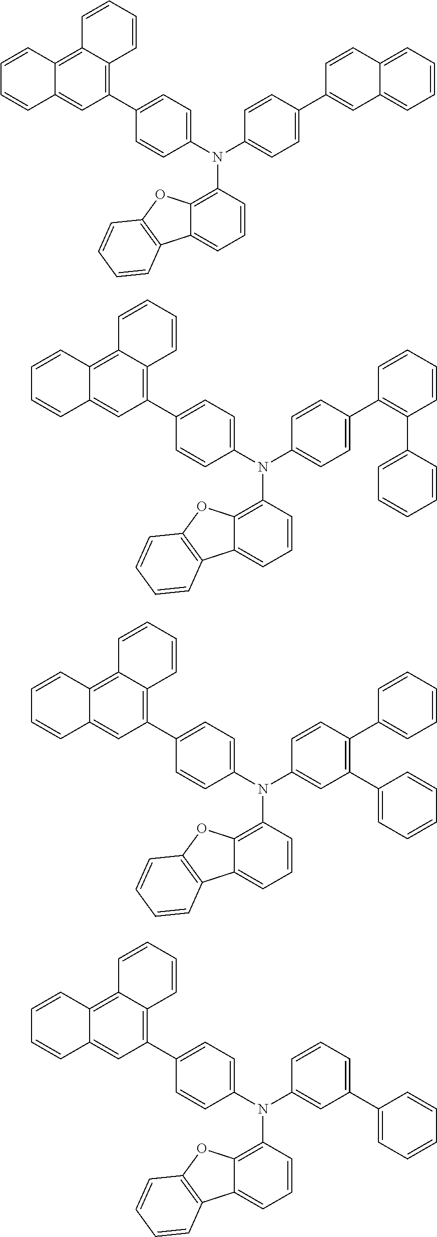 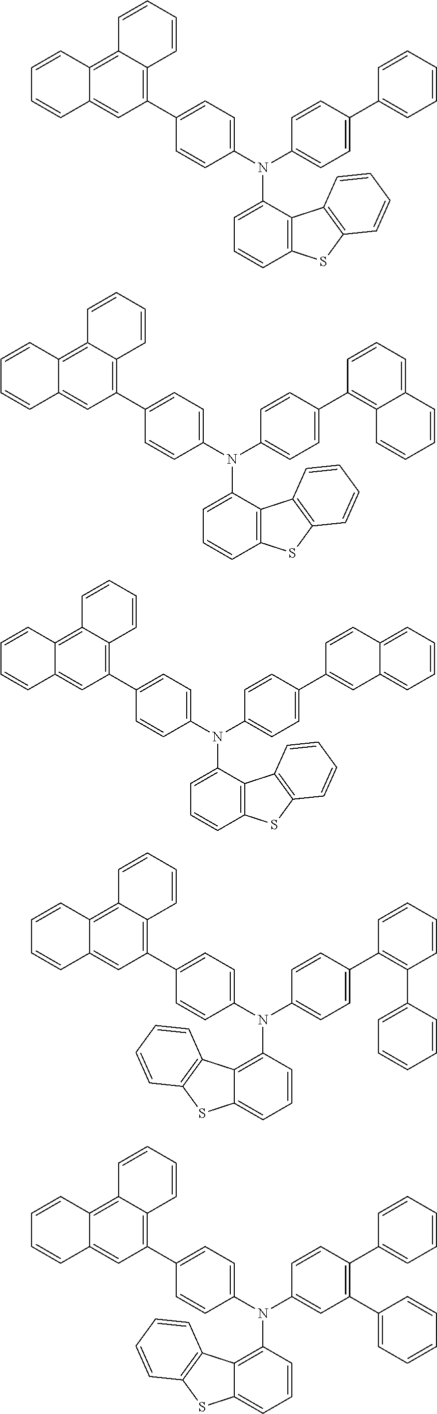 [SMILES: c1ccc(-c2ccc(N(c3ccc(-c4cc5ccccc5c5ccccc45)cc3)c3cccc4sc5ccccc5c34)cc2)cc1 ; c1ccc2c(-c3ccc(N(c4ccc(-c5cc6ccccc6c6ccccc56)cc4)c4cccc5sc6ccccc6c45)cc3)cccc2c1 ; c1ccc2cc(-c3ccc(N(c4ccc(-c5cc6ccccc6c6ccccc56)cc4)c4cccc5sc6ccccc6c45)cc3)ccc2c1 ; c1ccc(-c2ccccc2-c2ccc(N(c3ccc(-c4cc5ccccc5c5ccccc45)cc3)c3cccc4sc5ccccc5c34)cc2)cc1 ; c1ccc(-c2ccc(N(c3ccc(-c4cc5ccccc5c5ccccc45)cc3)c3cccc4sc5ccccc5c34)cc2-c2ccccc2)cc1] 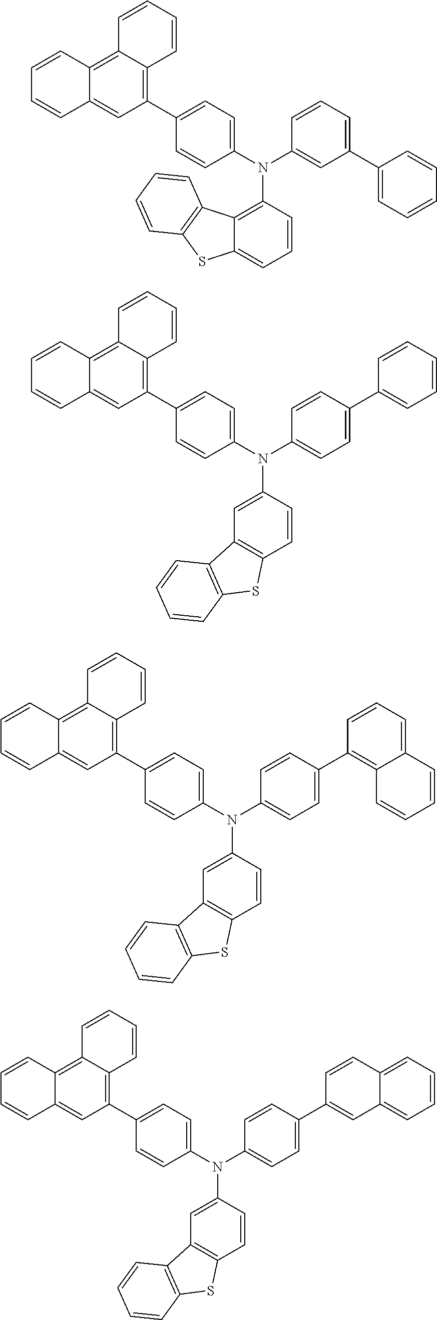 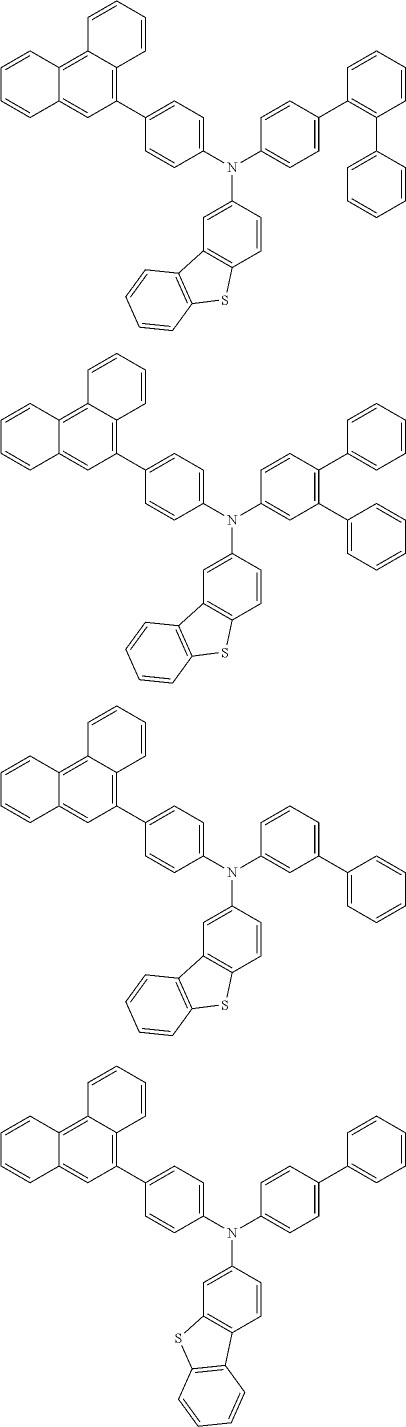 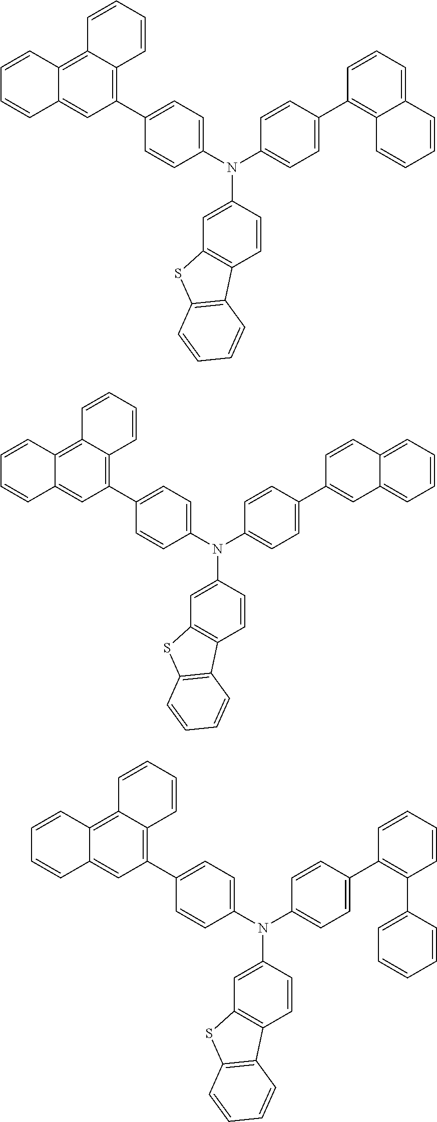 [SMILES: c1ccc2c(-c3ccc(N(c4ccc(-c5cc6ccccc6c6ccccc56)cc4)c4ccc5c(c4)sc4ccccc45)cc3)cccc2c1 ; c1ccc2cc(-c3ccc(N(c4ccc(-c5cc6ccccc6c6ccccc56)cc4)c4ccc5c(c4)sc4ccccc45)cc3)ccc2c1 ; c1ccc(-c2ccccc2-c2ccc(N(c3ccc(-c4cc5ccccc5c5ccccc45)cc3)c3ccc4c(c3)sc3ccccc34)cc2)cc1] 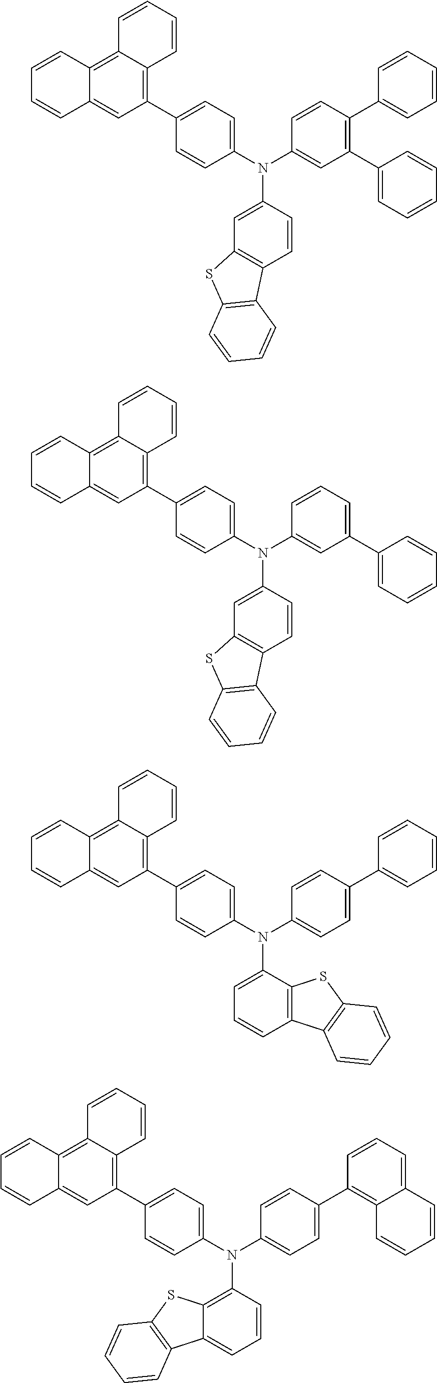 [SMILES: c1ccc(-c2ccc(N(c3ccc(-c4cc5ccccc5c5ccccc45)cc3)c3ccc4c(c3)sc3ccccc34)cc2-c2ccccc2)cc1 ; c1ccc(-c2cccc(N(c3ccc(-c4cc5ccccc5c5ccccc45)cc3)c3ccc4c(c3)sc3ccccc34)c2)cc1 ; c1ccc(-c2ccc(N(c3ccc(-c4cc5ccccc5c5ccccc45)cc3)c3cccc4c3sc3ccccc34)cc2)cc1 ; c1ccc2c(-c3ccc(N(c4ccc(-c5cc6ccccc6c6ccccc56)cc4)c4cccc5c4sc4ccccc45)cc3)cccc2c1] 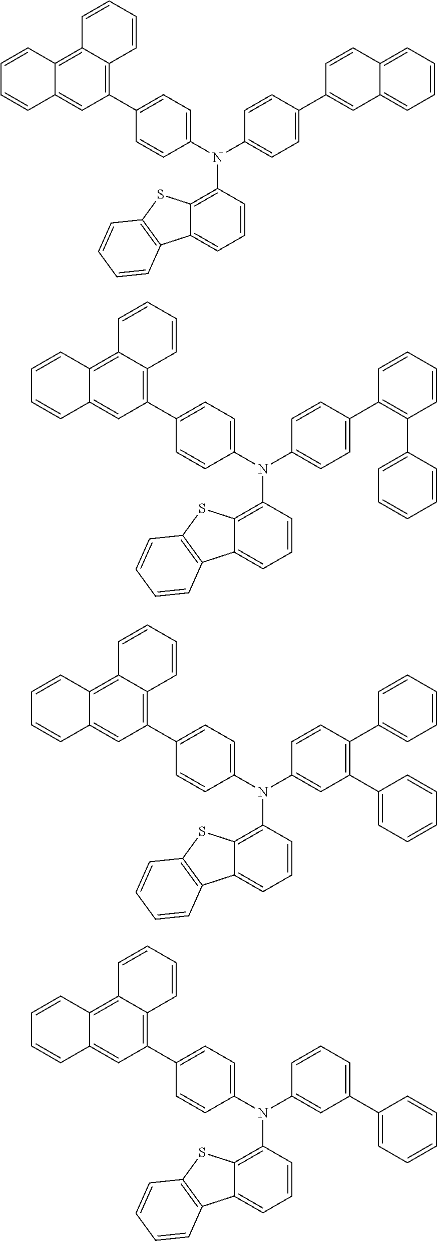 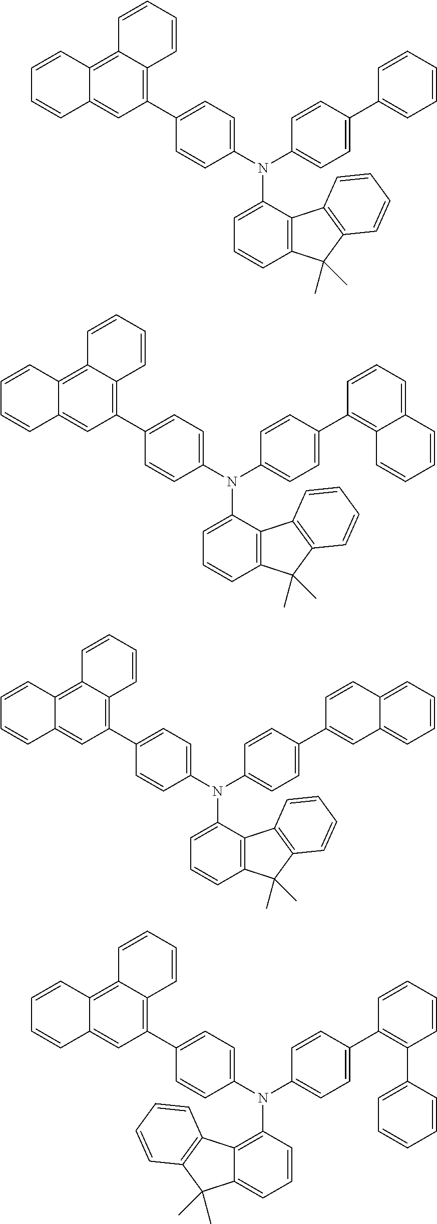 [SMILES: CC1(C)c2ccccc2-c2c(N(c3ccc(-c4ccccc4)cc3)c3ccc(-c4cc5ccccc5c5ccccc45)cc3)cccc21 ; CC1(C)c2ccccc2-c2c(N(c3ccc(-c4cccc5ccccc45)cc3)c3ccc(-c4cc5ccccc5c5ccccc45)cc3)cccc21 ; CC1(C)c2ccccc2-c2c(N(c3ccc(-c4ccc5ccccc5c4)cc3)c3ccc(-c4cc5ccccc5c5ccccc45)cc3)cccc21 ; CC1(C)c2ccccc2-c2c(N(c3ccc(-c4ccccc4-c4ccccc4)cc3)c3ccc(-c4cc5ccccc5c5ccccc45)cc3)cccc21] 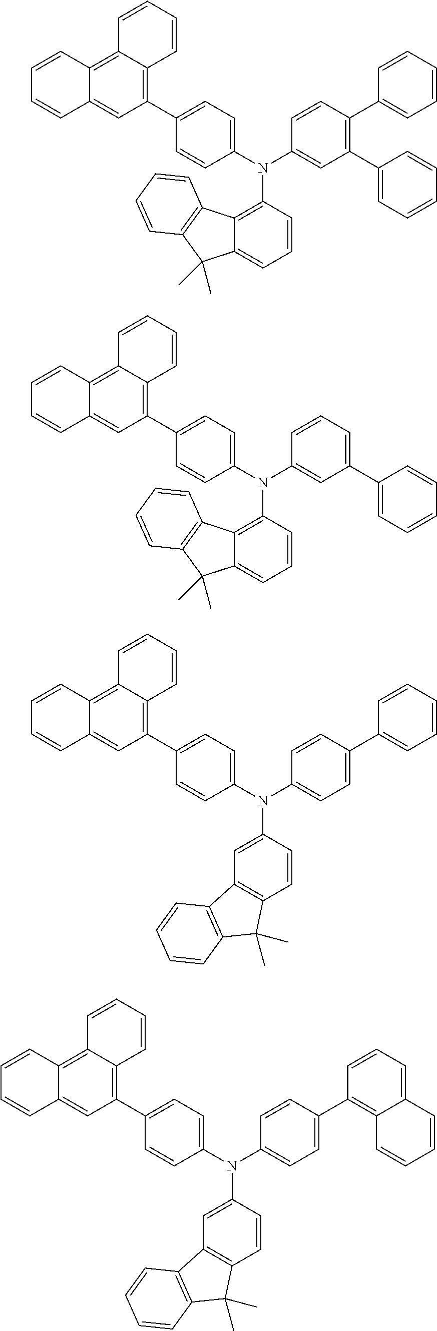 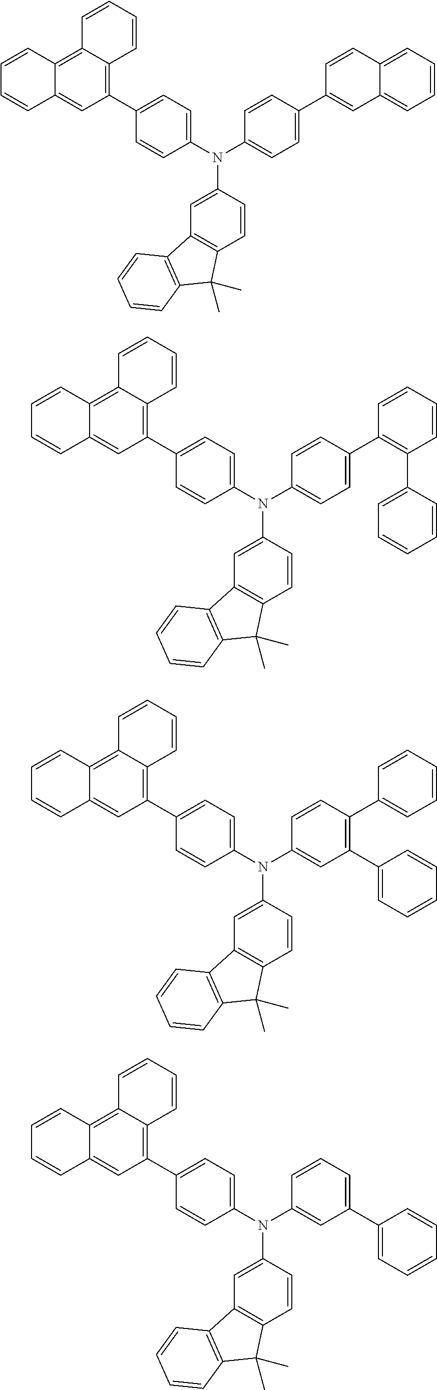 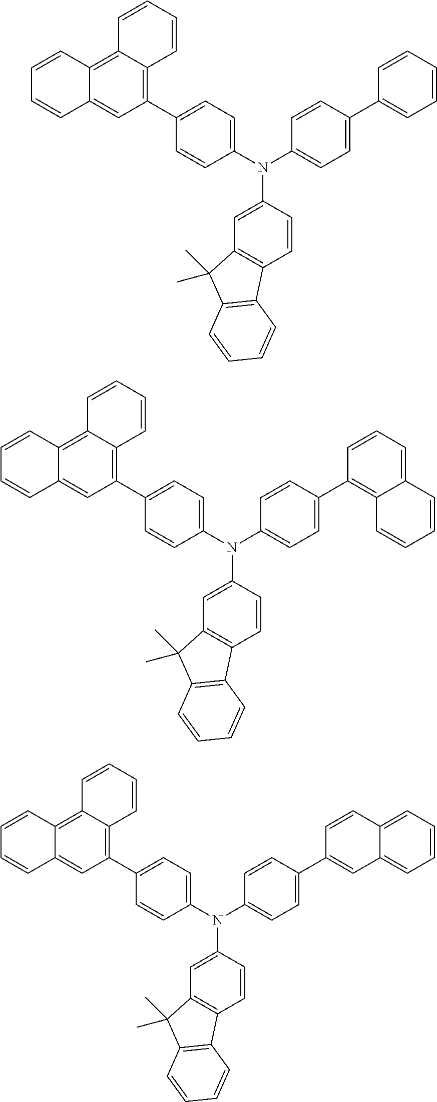 [SMILES: CC1(C)c2ccccc2-c2ccc(N(c3ccc(-c4ccccc4)cc3)c3ccc(-c4cc5ccccc5c5ccccc45)cc3)cc21 ; CC1(C)c2ccccc2-c2ccc(N(c3ccc(-c4cccc5ccccc45)cc3)c3ccc(-c4cc5ccccc5c5ccccc45)cc3)cc21 ; CC1(C)c2ccccc2-c2ccc(N(c3ccc(-c4ccc5ccccc5c4)cc3)c3ccc(-c4cc5ccccc5c5ccccc45)cc3)cc21] 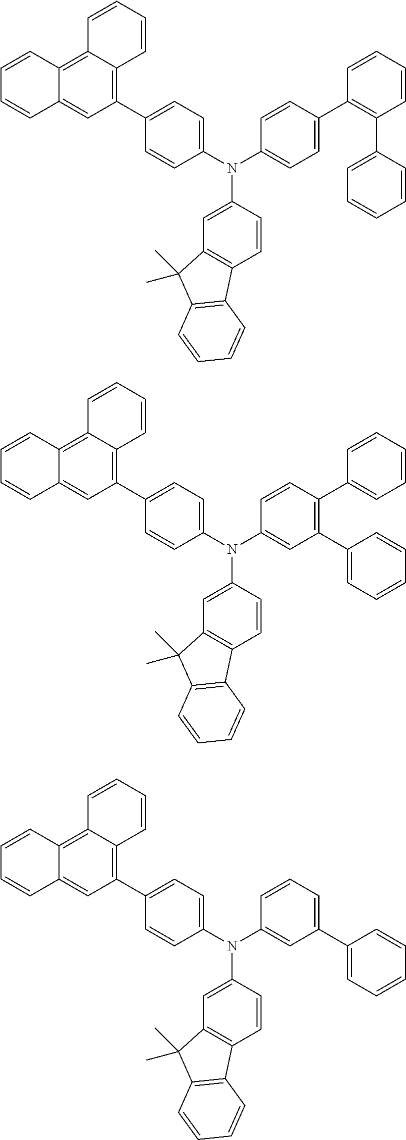 [SMILES: CC1(C)c2ccccc2-c2ccc(N(c3ccc(-c4ccccc4-c4ccccc4)cc3)c3ccc(-c4cc5ccccc5c5ccccc45)cc3)cc21 ; CC1(C)c2ccccc2-c2ccc(N(c3ccc(-c4cc5ccccc5c5ccccc45)cc3)c3ccc(-c4ccccc4)c(-c4ccccc4)c3)cc21 ; CC1(C)c2ccccc2-c2ccc(N(c3ccc(-c4cc5ccccc5c5ccccc45)cc3)c3cccc(-c4ccccc4)c3)cc21] 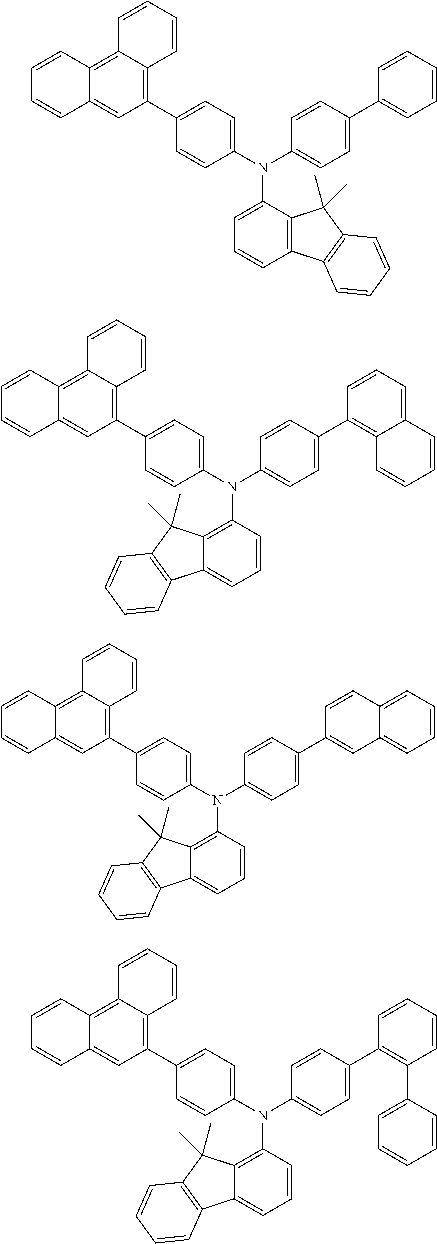 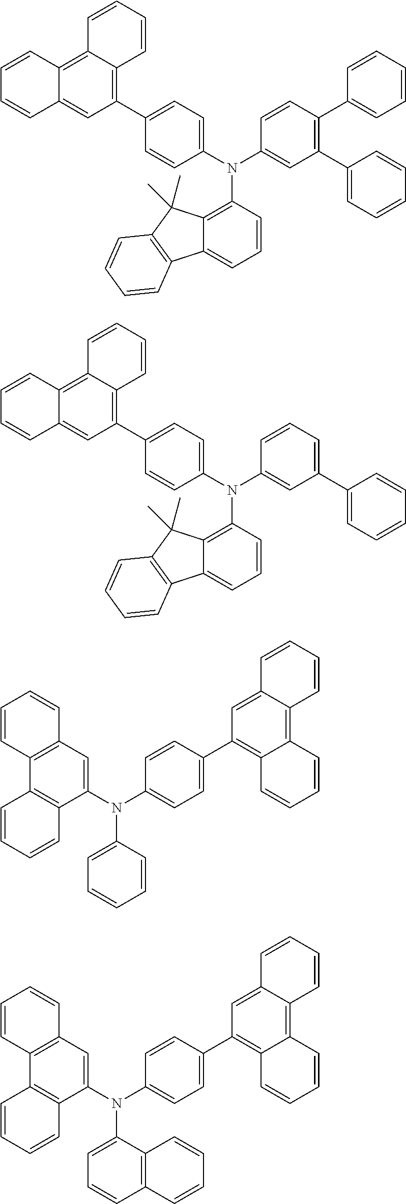 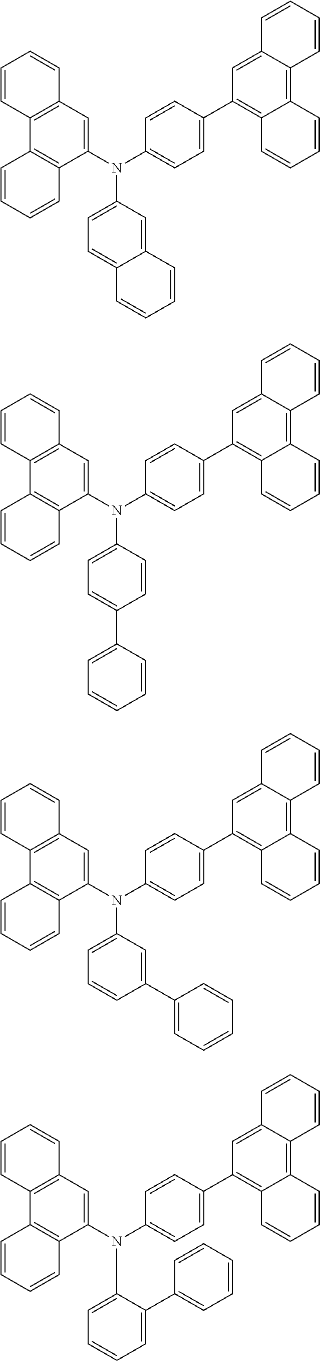 [SMILES: c1ccc2cc(N(c3ccc(-c4cc5ccccc5c5ccccc45)cc3)c3cc4ccccc4c4ccccc34)ccc2c1 ; c1ccc(-c2ccc(N(c3ccc(-c4cc5ccccc5c5ccccc45)cc3)c3cc4ccccc4c4ccccc34)cc2)cc1 ; c1ccc(-c2cccc(N(c3ccc(-c4cc5ccccc5c5ccccc45)cc3)c3cc4ccccc4c4ccccc34)c2)cc1 ; c1ccc(-c2ccccc2N(c2ccc(-c3cc4ccccc4c4ccccc34)cc2)c2cc3ccccc3c3ccccc23)cc1] 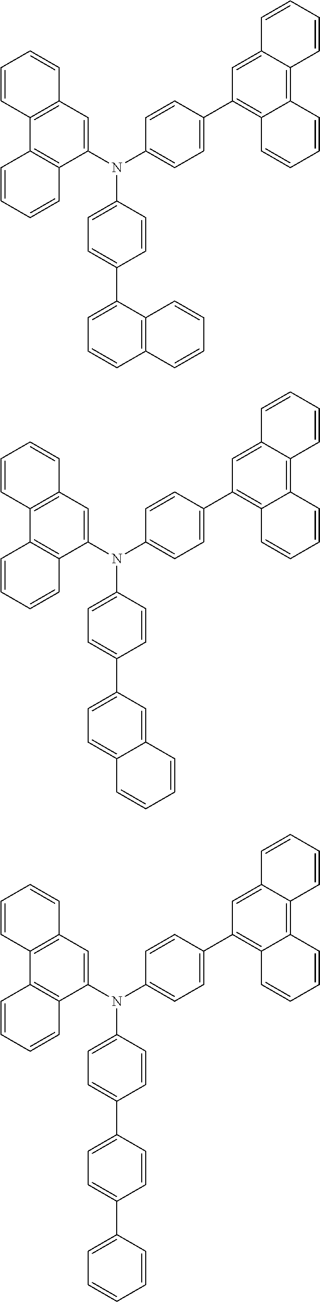 [SMILES: c1ccc2c(-c3ccc(N(c4ccc(-c5cc6ccccc6c6ccccc56)cc4)c4cc5ccccc5c5ccccc45)cc3)cccc2c1 ; c1ccc2cc(-c3ccc(N(c4ccc(-c5cc6ccccc6c6ccccc56)cc4)c4cc5ccccc5c5ccccc45)cc3)ccc2c1 ; c1ccc(-c2ccc(-c3ccc(N(c4ccc(-c5cc6ccccc6c6ccccc56)cc4)c4cc5ccccc5c5ccccc45)cc3)cc2)cc1] 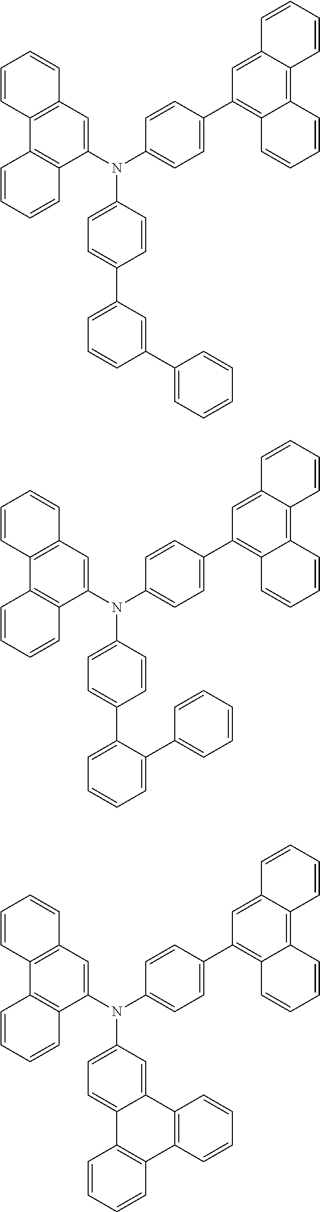 [SMILES: c1ccc(-c2cccc(-c3ccc(N(c4ccc(-c5cc6ccccc6c6ccccc56)cc4)c4cc5ccccc5c5ccccc45)cc3)c2)cc1 ; c1ccc(-c2ccccc2-c2ccc(N(c3ccc(-c4cc5ccccc5c5ccccc45)cc3)c3cc4ccccc4c4ccccc34)cc2)cc1 ; c1ccc2c(c1)cc(-c1ccc(N(c3ccc4c5ccccc5c5ccccc5c4c3)c3cc4ccccc4c4ccccc34)cc1)c1ccccc12] 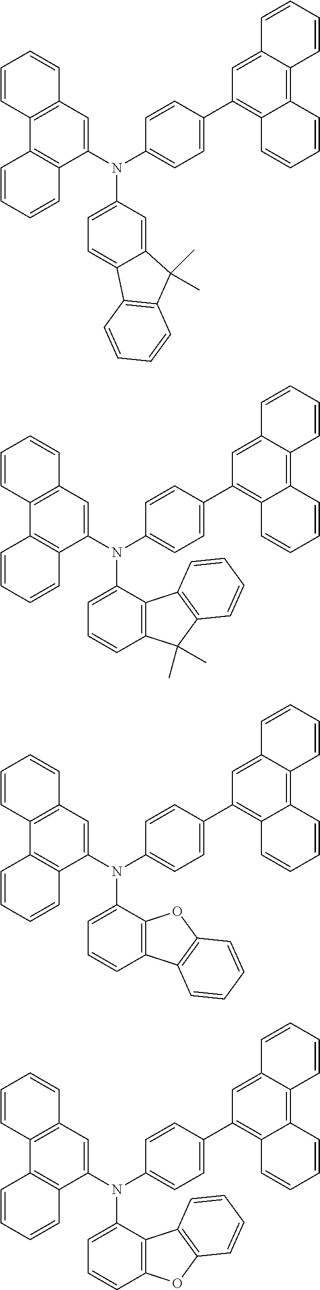 [SMILES: CC1(C)c2ccccc2-c2ccc(N(c3ccc(-c4cc5ccccc5c5ccccc45)cc3)c3cc4ccccc4c4ccccc34)cc21 ; CC1(C)c2ccccc2-c2c(N(c3ccc(-c4cc5ccccc5c5ccccc45)cc3)c3cc4ccccc4c4ccccc34)cccc21 ; c1ccc2c(c1)cc(-c1ccc(N(c3cc4ccccc4c4ccccc34)c3cccc4c3oc3ccccc34)cc1)c1ccccc12 ; c1ccc2c(c1)cc(-c1ccc(N(c3cc4ccccc4c4ccccc34)c3cccc4oc5ccccc5c34)cc1)c1ccccc12] 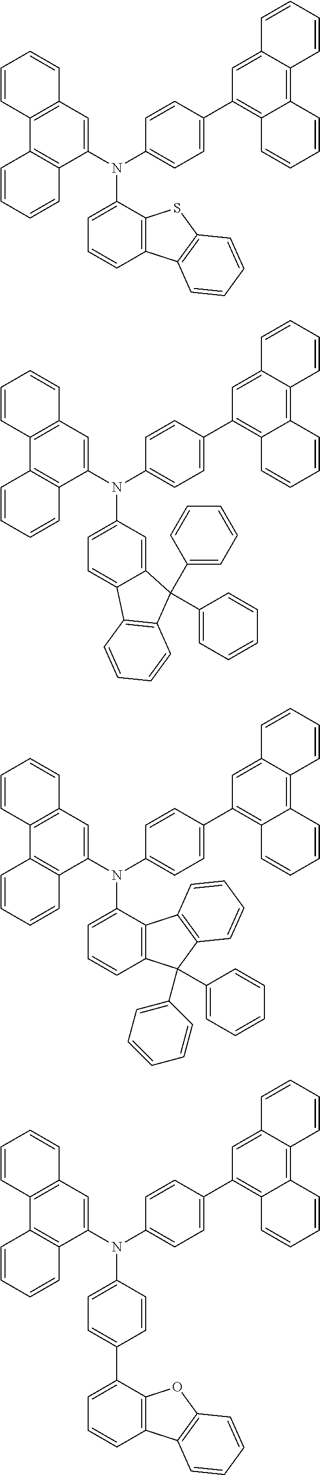 [SMILES: c1ccc2c(c1)cc(-c1ccc(N(c3cc4ccccc4c4ccccc34)c3cccc4c3sc3ccccc34)cc1)c1ccccc12 ; c1ccc(C2(c3ccccc3)c3ccccc3-c3ccc(N(c4ccc(-c5cc6ccccc6c6ccccc56)cc4)c4cc5ccccc5c5ccccc45)cc32)cc1 ; c1ccc(C2(c3ccccc3)c3ccccc3-c3c(N(c4ccc(-c5cc6ccccc6c6ccccc56)cc4)c4cc5ccccc5c5ccccc45)cccc32)cc1 ; c1ccc2c(c1)cc(-c1ccc(N(c3ccc(-c4cccc5c4oc4ccccc45)cc3)c3cc4ccccc4c4ccccc34)cc1)c1ccccc12] 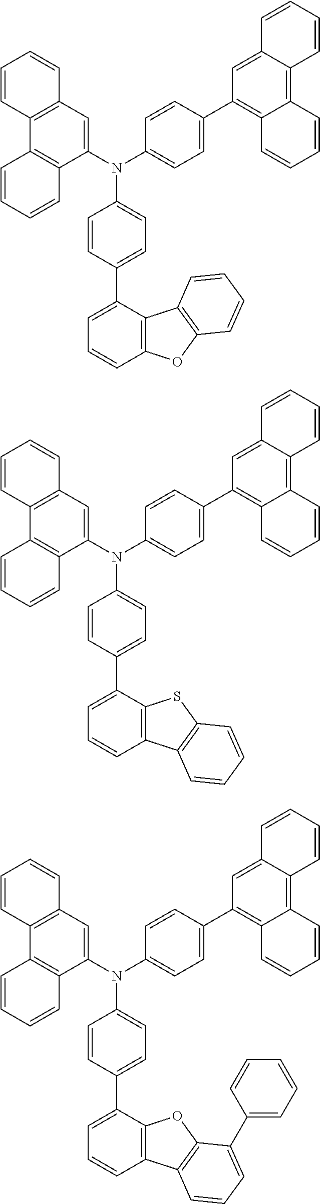 [SMILES: c1ccc2c(c1)cc(-c1ccc(N(c3ccc(-c4cccc5oc6ccccc6c45)cc3)c3cc4ccccc4c4ccccc34)cc1)c1ccccc12 ; c1ccc2c(c1)cc(-c1ccc(N(c3ccc(-c4cccc5c4sc4ccccc45)cc3)c3cc4ccccc4c4ccccc34)cc1)c1ccccc12 ; c1ccc(-c2cccc3c2oc2c(-c4ccc(N(c5ccc(-c6cc7ccccc7c7ccccc67)cc5)c5cc6ccccc6c6ccccc56)cc4)cccc23)cc1] 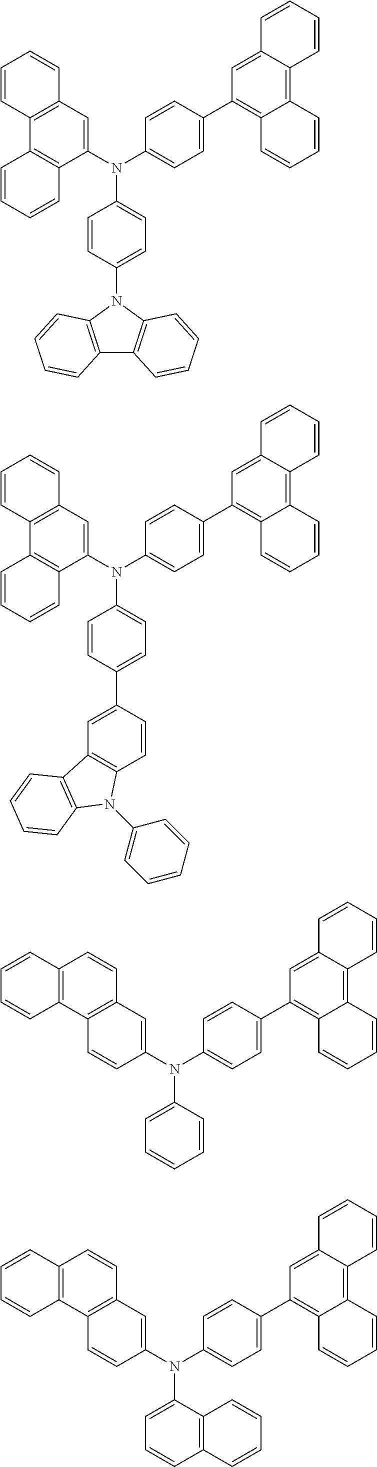 [SMILES: c1ccc2c(c1)cc(-c1ccc(N(c3ccc(-n4c5ccccc5c5ccccc54)cc3)c3cc4ccccc4c4ccccc34)cc1)c1ccccc12 ; c1ccc(-n2c3ccccc3c3cc(-c4ccc(N(c5ccc(-c6cc7ccccc7c7ccccc67)cc5)c5cc6ccccc6c6ccccc56)cc4)ccc32)cc1 ; c1ccc(N(c2ccc(-c3cc4ccccc4c4ccccc34)cc2)c2ccc3c(ccc4ccccc43)c2)cc1 ; c1ccc2c(N(c3ccc(-c4cc5ccccc5c5ccccc45)cc3)c3ccc4c(ccc5ccccc54)c3)cccc2c1] 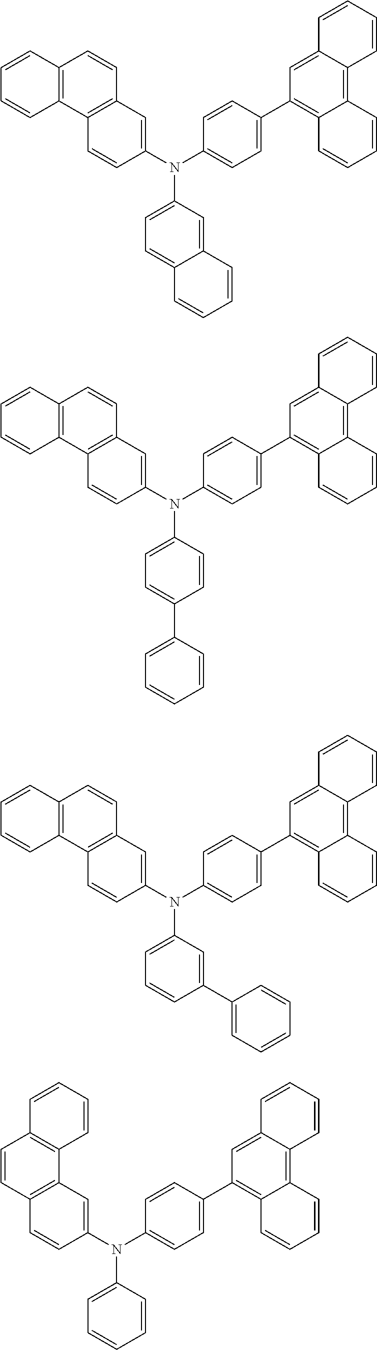 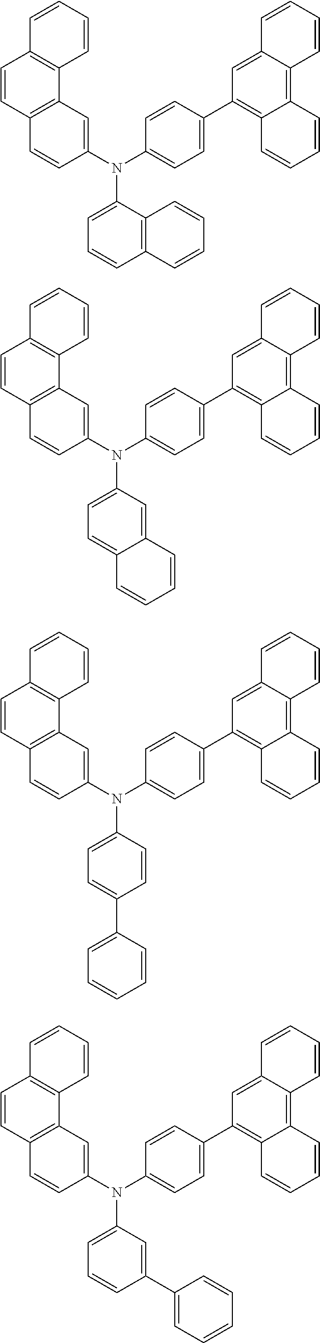 [SMILES: c1ccc2c(N(c3ccc(-c4cc5ccccc5c5ccccc45)cc3)c3ccc4ccc5ccccc5c4c3)cccc2c1 ; c1ccc2cc(N(c3ccc(-c4cc5ccccc5c5ccccc45)cc3)c3ccc4ccc5ccccc5c4c3)ccc2c1 ; c1ccc(-c2ccc(N(c3ccc(-c4cc5ccccc5c5ccccc45)cc3)c3ccc4ccc5ccccc5c4c3)cc2)cc1 ; c1ccc(-c2cccc(N(c3ccc(-c4cc5ccccc5c5ccccc45)cc3)c3ccc4ccc5ccccc5c4c3)c2)cc1] 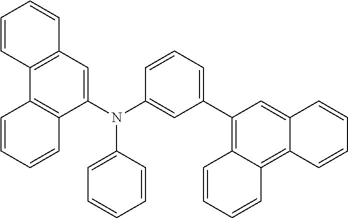 [SMILES: c1ccc(N(c2cccc(-c3cc4ccccc4c4ccccc34)c2)c2cc3ccccc3c3ccccc23)cc1] 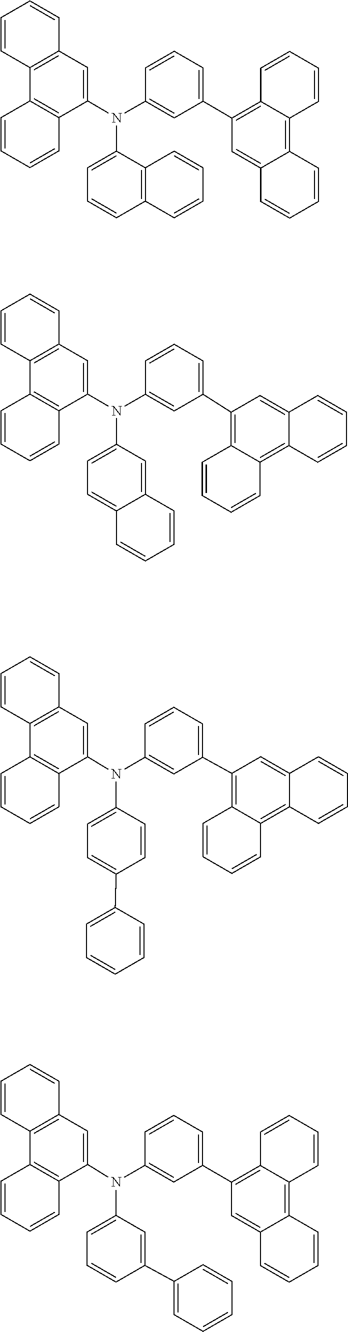 [SMILES: c1cc(-c2cc3ccccc3c3ccccc23)cc(N(c2cccc3ccccc23)c2cc3ccccc3c3ccccc23)c1 ; c1cc(-c2cc3ccccc3c3ccccc23)cc(N(c2ccc3ccccc3c2)c2cc3ccccc3c3ccccc23)c1 ; c1ccc(-c2ccc(N(c3cccc(-c4cc5ccccc5c5ccccc45)c3)c3cc4ccccc4c4ccccc34)cc2)cc1 ; c1ccc(-c2cccc(N(c3cccc(-c4cc5ccccc5c5ccccc45)c3)c3cc4ccccc4c4ccccc34)c2)cc1] 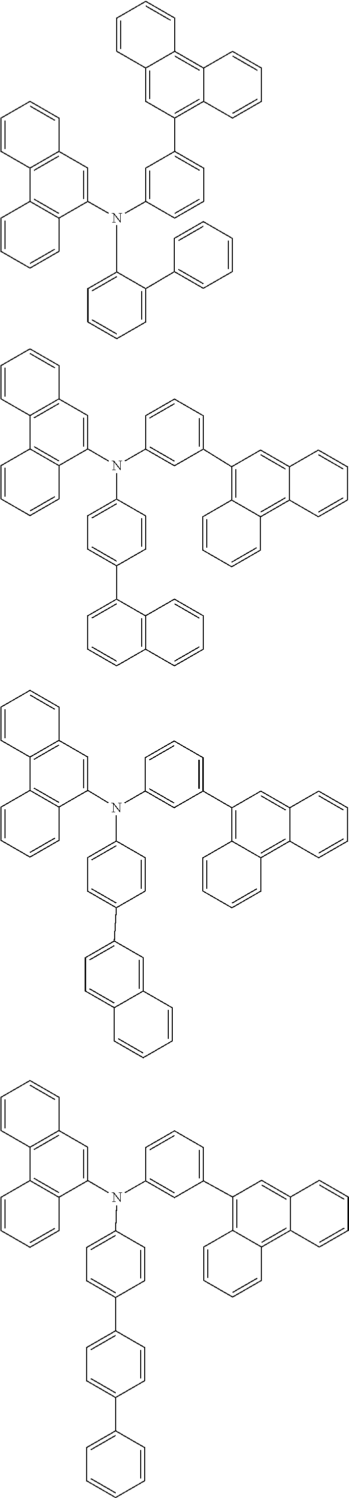 [SMILES: c1ccc(-c2ccccc2N(c2cccc(-c3cc4ccccc4c4ccccc34)c2)c2cc3ccccc3c3ccccc23)cc1 ; c1cc(-c2cc3ccccc3c3ccccc23)cc(N(c2ccc(-c3cccc4ccccc34)cc2)c2cc3ccccc3c3ccccc23)c1 ; c1cc(-c2cc3ccccc3c3ccccc23)cc(N(c2ccc(-c3ccc4ccccc4c3)cc2)c2cc3ccccc3c3ccccc23)c1 ; c1ccc(-c2ccc(-c3ccc(N(c4cccc(-c5cc6ccccc6c6ccccc56)c4)c4cc5ccccc5c5ccccc45)cc3)cc2)cc1] 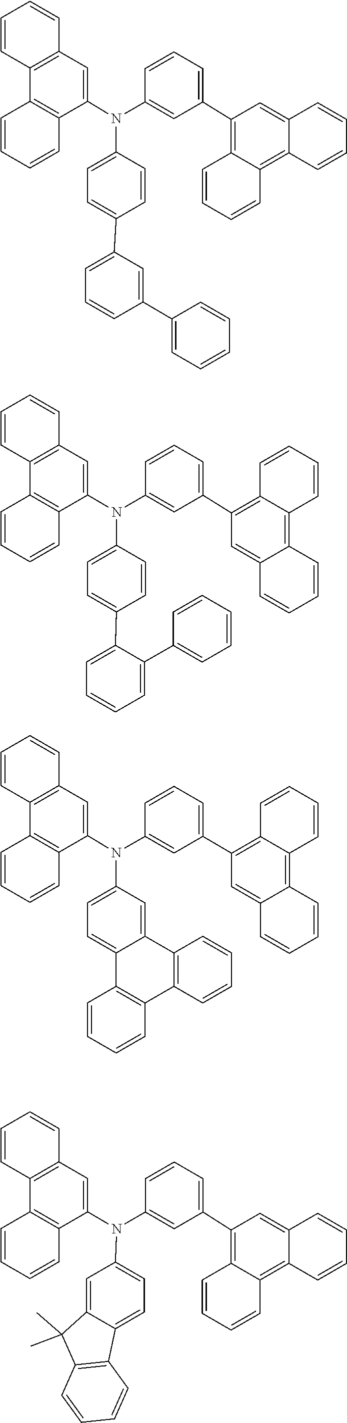 [SMILES: c1ccc(-c2cccc(-c3ccc(N(c4cccc(-c5cc6ccccc6c6ccccc56)c4)c4cc5ccccc5c5ccccc45)cc3)c2)cc1 ; c1ccc(-c2ccccc2-c2ccc(N(c3cccc(-c4cc5ccccc5c5ccccc45)c3)c3cc4ccccc4c4ccccc34)cc2)cc1 ; c1cc(-c2cc3ccccc3c3ccccc23)cc(N(c2ccc3c4ccccc4c4ccccc4c3c2)c2cc3ccccc3c3ccccc23)c1 ; CC1(C)c2ccccc2-c2ccc(N(c3cccc(-c4cc5ccccc5c5ccccc45)c3)c3cc4ccccc4c4ccccc34)cc21] 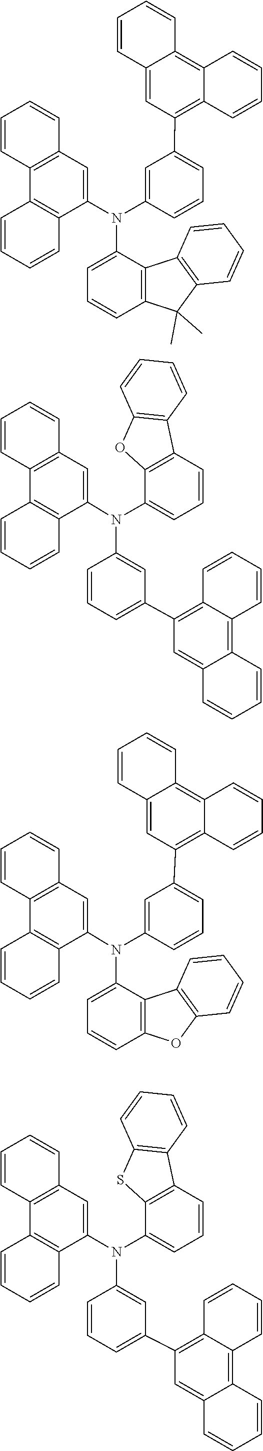 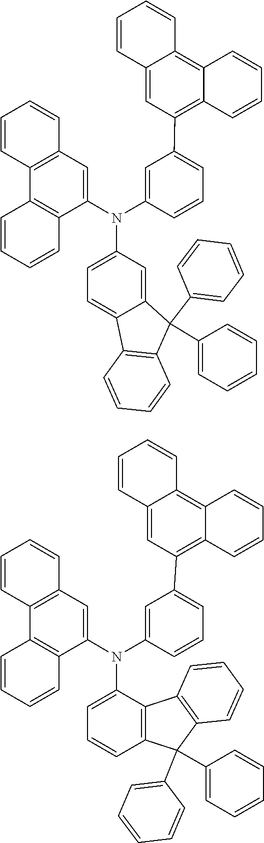 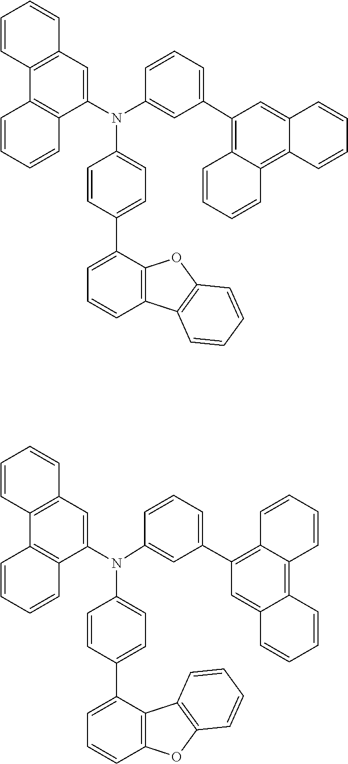 [SMILES: c1cc(-c2cc3ccccc3c3ccccc23)cc(N(c2ccc(-c3cccc4c3oc3ccccc34)cc2)c2cc3ccccc3c3ccccc23)c1 ; c1cc(-c2cc3ccccc3c3ccccc23)cc(N(c2ccc(-c3cccc4oc5ccccc5c34)cc2)c2cc3ccccc3c3ccccc23)c1] 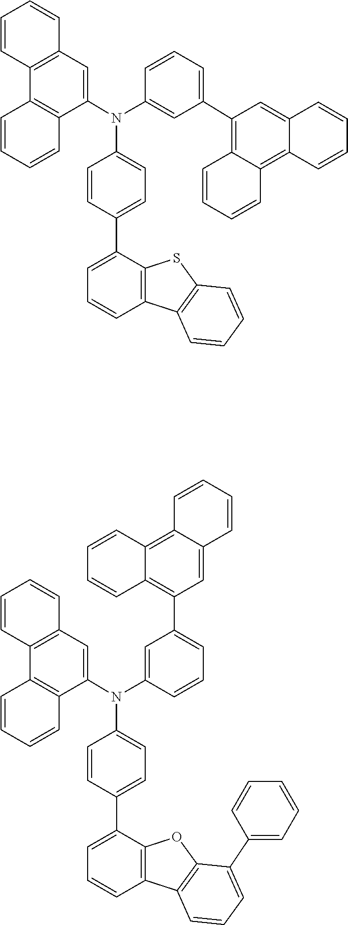 [SMILES: c1cc(-c2cc3ccccc3c3ccccc23)cc(N(c2ccc(-c3cccc4c3sc3ccccc34)cc2)c2cc3ccccc3c3ccccc23)c1 ; c1ccc(-c2cccc3c2oc2c(-c4ccc(N(c5cccc(-c6cc7ccccc7c7ccccc67)c5)c5cc6ccccc6c6ccccc56)cc4)cccc23)cc1] 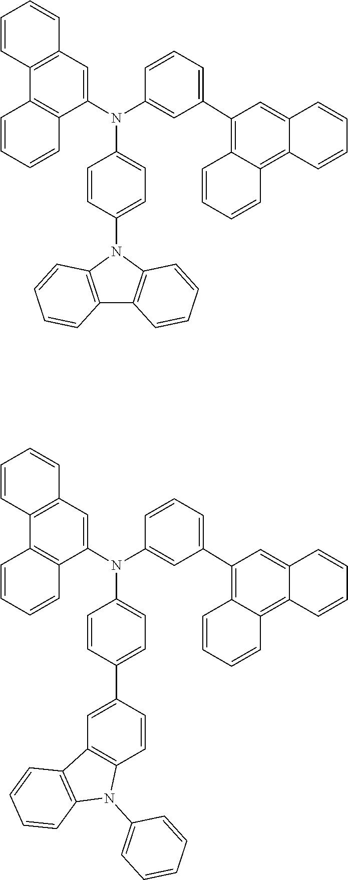 [SMILES: c1cc(-c2cc3ccccc3c3ccccc23)cc(N(c2ccc(-n3c4ccccc4c4ccccc43)cc2)c2cc3ccccc3c3ccccc23)c1 ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1] 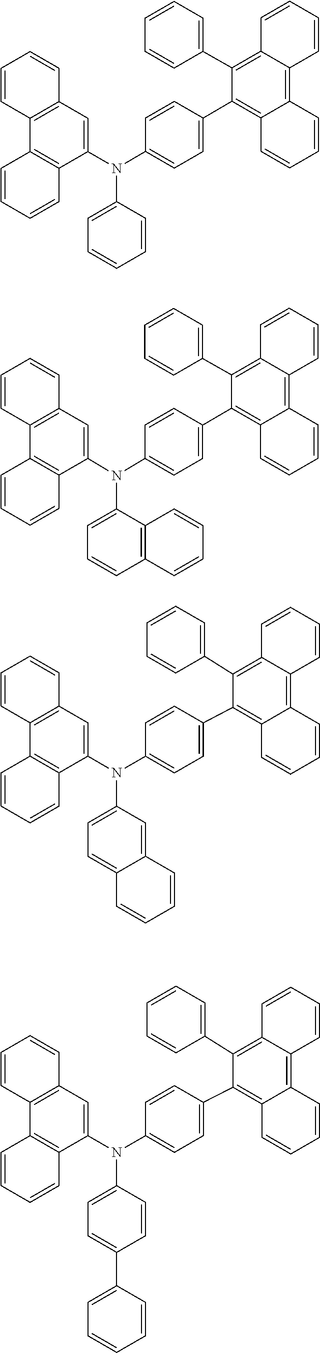 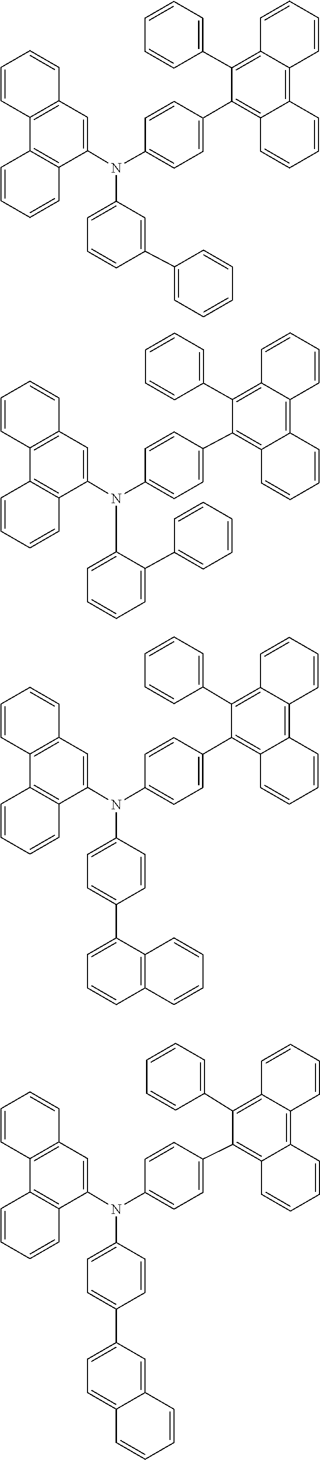 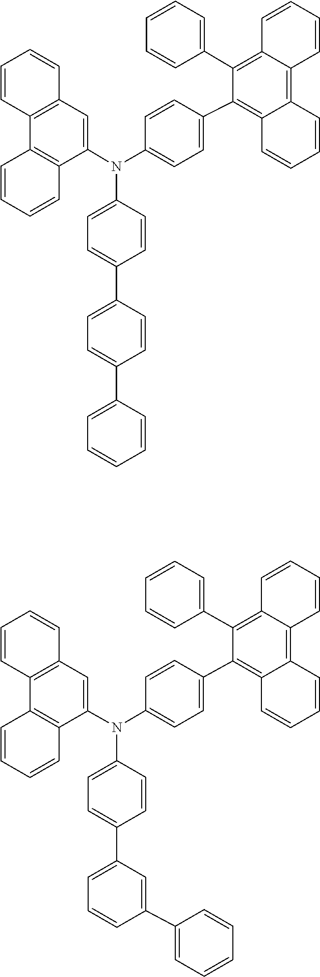 [SMILES: c1ccc(-c2ccc(-c3ccc(N(c4ccc(-c5c(-c6ccccc6)c6ccccc6c6ccccc56)cc4)c4cc5ccccc5c5ccccc45)cc3)cc2)cc1 ; c1ccc(-c2cccc(-c3ccc(N(c4ccc(-c5c(-c6ccccc6)c6ccccc6c6ccccc56)cc4)c4cc5ccccc5c5ccccc45)cc3)c2)cc1] 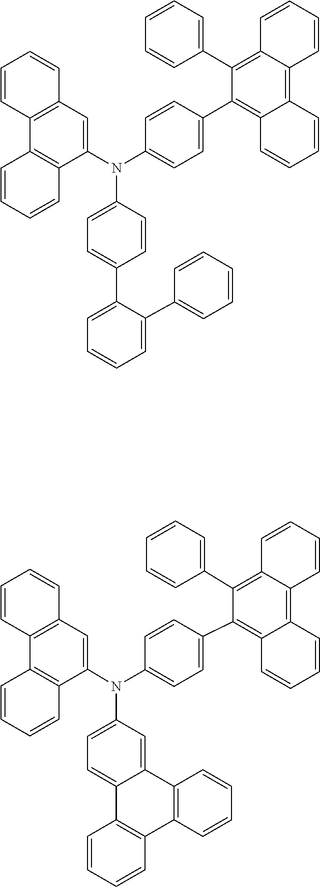 [SMILES: c1ccc(-c2ccccc2-c2ccc(N(c3ccc(-c4c(-c5ccccc5)c5ccccc5c5ccccc45)cc3)c3cc4ccccc4c4ccccc34)cc2)cc1 ; c1ccc(-c2c(-c3ccc(N(c4ccc5c6ccccc6c6ccccc6c5c4)c4cc5ccccc5c5ccccc45)cc3)c3ccccc3c3ccccc23)cc1] 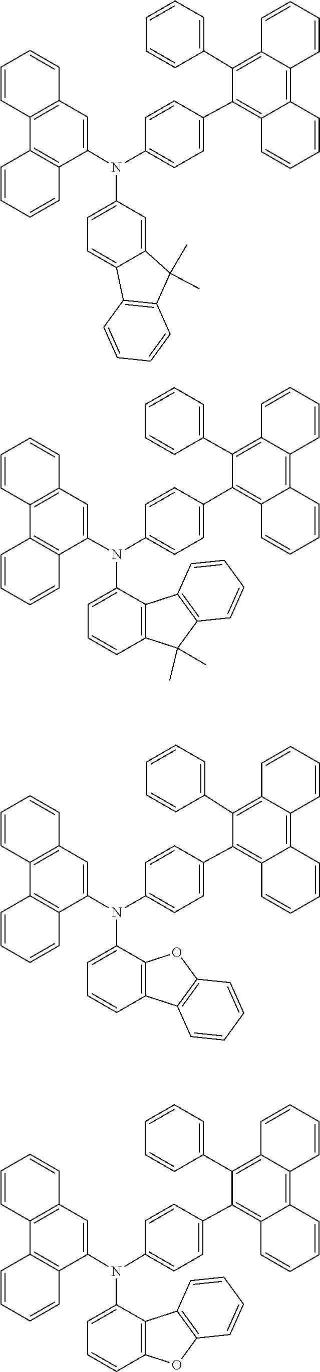 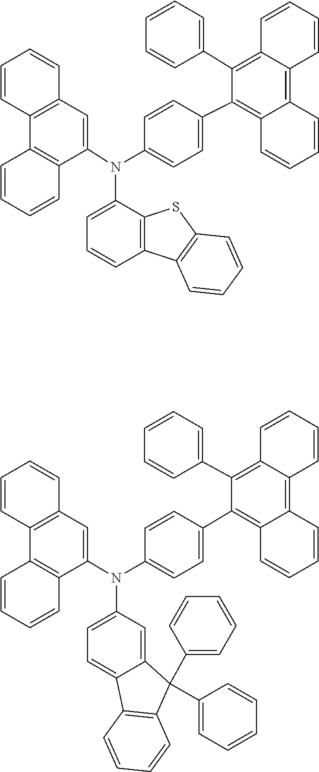 [SMILES: c1ccc(-c2c(-c3ccc(N(c4cc5ccccc5c5ccccc45)c4cccc5c4sc4ccccc45)cc3)c3ccccc3c3ccccc23)cc1 ; c1ccc(-c2c(-c3ccc(N(c4ccc5c(c4)C(c4ccccc4)(c4ccccc4)c4ccccc4-5)c4cc5ccccc5c5ccccc45)cc3)c3ccccc3c3ccccc23)cc1] 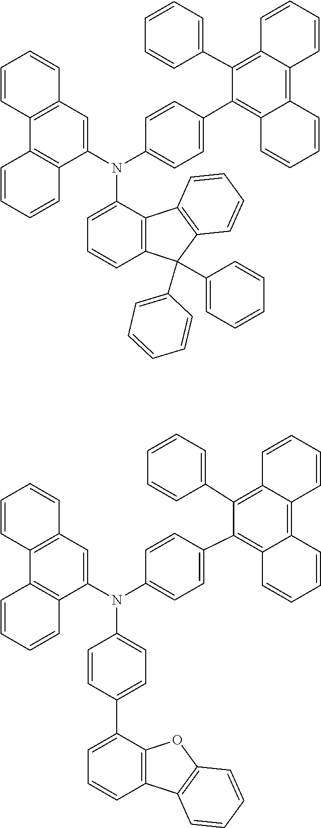 [SMILES: c1ccc(-c2c(-c3ccc(N(c4cccc5c4-c4ccccc4C5(c4ccccc4)c4ccccc4)c4cc5ccccc5c5ccccc45)cc3)c3ccccc3c3ccccc23)cc1 ; c1ccc(-c2c(-c3ccc(N(c4ccc(-c5cccc6c5oc5ccccc56)cc4)c4cc5ccccc5c5ccccc45)cc3)c3ccccc3c3ccccc23)cc1] 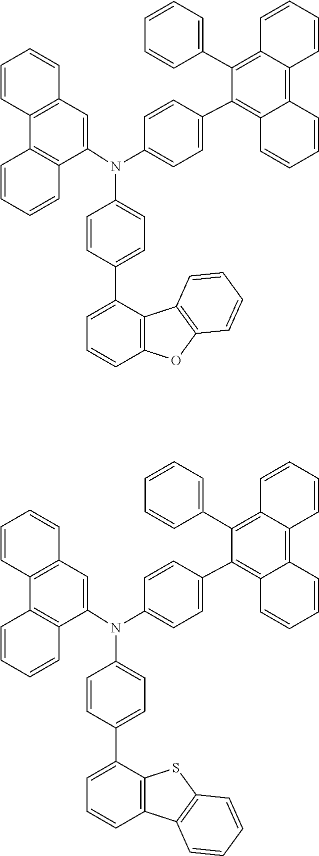 [SMILES: c1ccc(-c2c(-c3ccc(N(c4ccc(-c5cccc6oc7ccccc7c56)cc4)c4cc5ccccc5c5ccccc45)cc3)c3ccccc3c3ccccc23)cc1 ; c1ccc(-c2c(-c3ccc(N(c4ccc(-c5cccc6c5sc5ccccc56)cc4)c4cc5ccccc5c5ccccc45)cc3)c3ccccc3c3ccccc23)cc1] 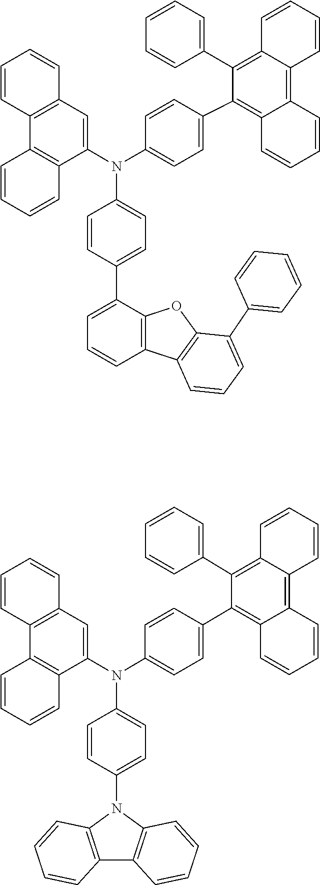 [SMILES: c1ccc(-c2c(-c3ccc(N(c4ccc(-c5cccc6c5oc5c(-c7ccccc7)cccc56)cc4)c4cc5ccccc5c5ccccc45)cc3)c3ccccc3c3ccccc23)cc1 ; c1ccc(-c2c(-c3ccc(N(c4ccc(-n5c6ccccc6c6ccccc65)cc4)c4cc5ccccc5c5ccccc45)cc3)c3ccccc3c3ccccc23)cc1] 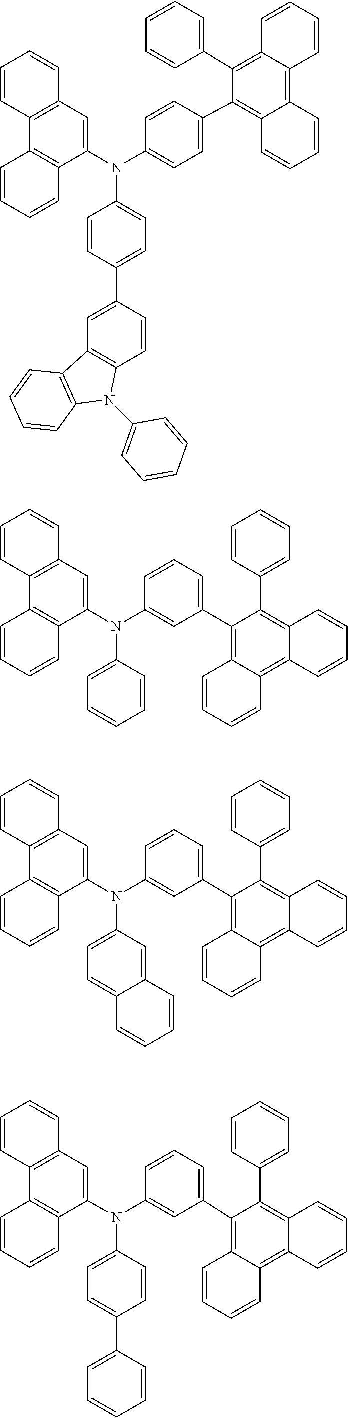 [SMILES: c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1 ; c1ccc(-c2c(-c3cccc(N(c4ccccc4)c4cc5ccccc5c5ccccc45)c3)c3ccccc3c3ccccc23)cc1 ; c1ccc(-c2c(-c3cccc(N(c4ccc5ccccc5c4)c4cc5ccccc5c5ccccc45)c3)c3ccccc3c3ccccc23)cc1 ; c1ccc(-c2ccc(N(c3cccc(-c4c(-c5ccccc5)c5ccccc5c5ccccc45)c3)c3cc4ccccc4c4ccccc34)cc2)cc1] 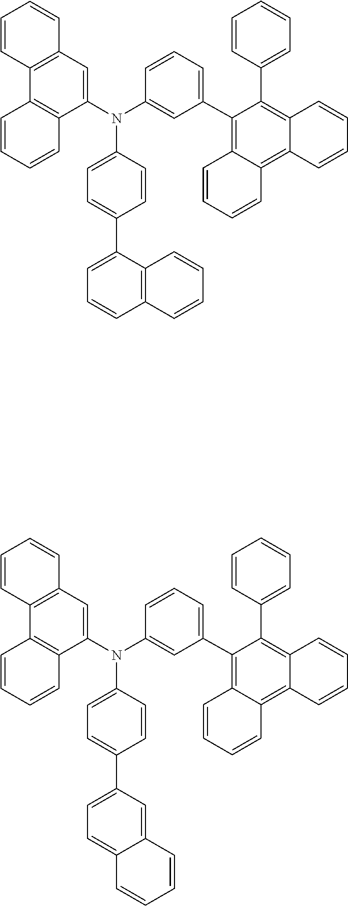 [SMILES: c1ccc(-c2c(-c3cccc(N(c4ccc(-c5cccc6ccccc56)cc4)c4cc5ccccc5c5ccccc45)c3)c3ccccc3c3ccccc23)cc1 ; c1ccc(-c2c(-c3cccc(N(c4ccc(-c5ccc6ccccc6c5)cc4)c4cc5ccccc5c5ccccc45)c3)c3ccccc3c3ccccc23)cc1] 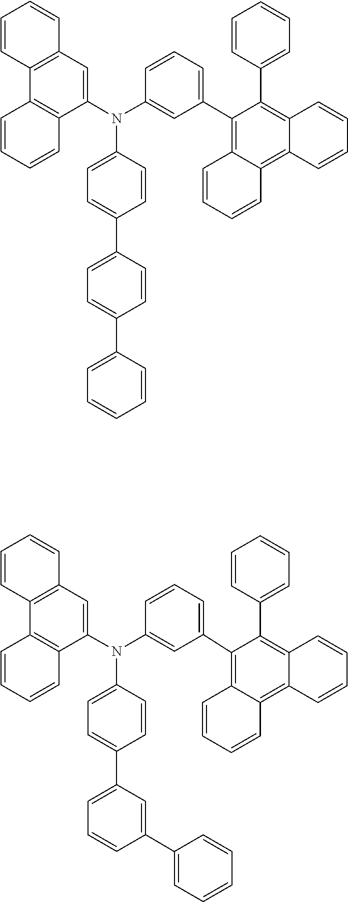 [SMILES: c1ccc(-c2ccc(-c3ccc(N(c4cccc(-c5c(-c6ccccc6)c6ccccc6c6ccccc56)c4)c4cc5ccccc5c5ccccc45)cc3)cc2)cc1 ; c1ccc(-c2cccc(-c3ccc(N(c4cccc(-c5c(-c6ccccc6)c6ccccc6c6ccccc56)c4)c4cc5ccccc5c5ccccc45)cc3)c2)cc1] 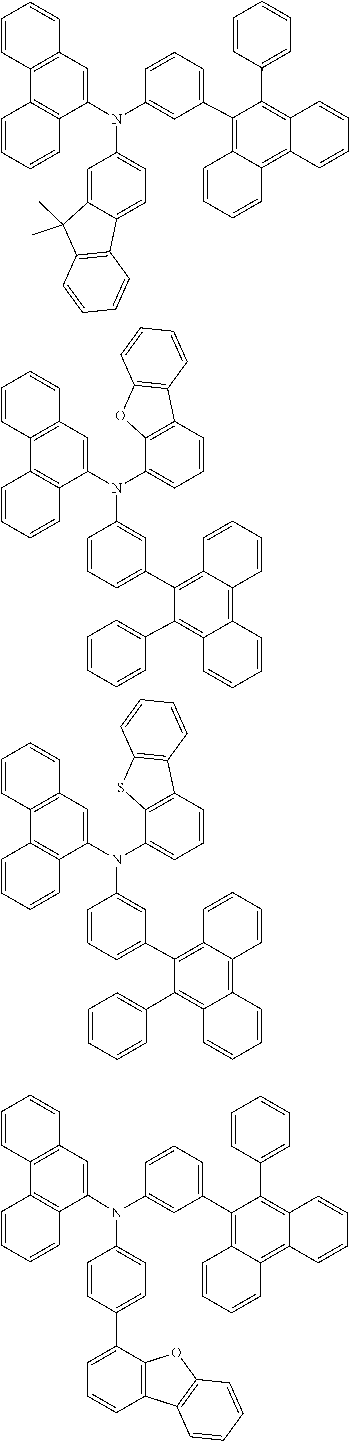 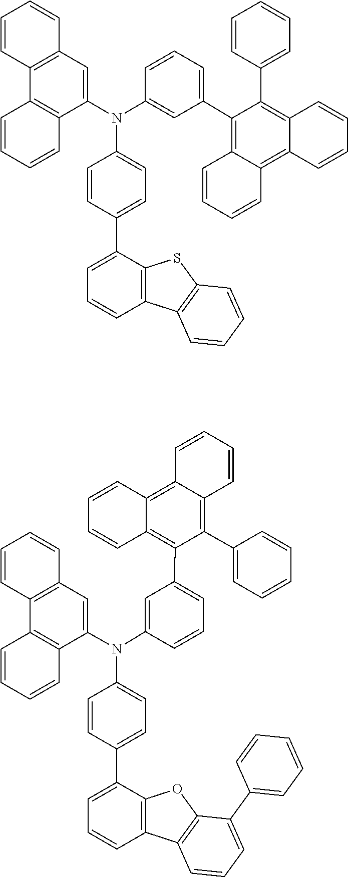 [SMILES: c1ccc(-c2c(-c3cccc(N(c4ccc(-c5cccc6c5sc5ccccc56)cc4)c4cc5ccccc5c5ccccc45)c3)c3ccccc3c3ccccc23)cc1 ; c1ccc(-c2c(-c3cccc(N(c4ccc(-c5cccc6c5oc5c(-c7ccccc7)cccc56)cc4)c4cc5ccccc5c5ccccc45)c3)c3ccccc3c3ccccc23)cc1] 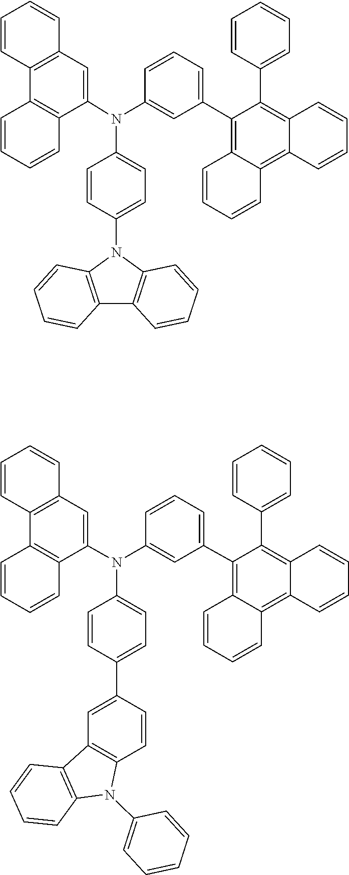 [SMILES: c1ccc(-c2c(-c3cccc(N(c4ccc(-n5c6ccccc6c6ccccc65)cc4)c4cc5ccccc5c5ccccc45)c3)c3ccccc3c3ccccc23)cc1 ; c1ccc(-c2c(-c3cccc(N(c4ccc(-c5ccc6c(c5)c5ccccc5n6-c5ccccc5)cc4)c4cc5ccccc5c5ccccc45)c3)c3ccccc3c3ccccc23)cc1] 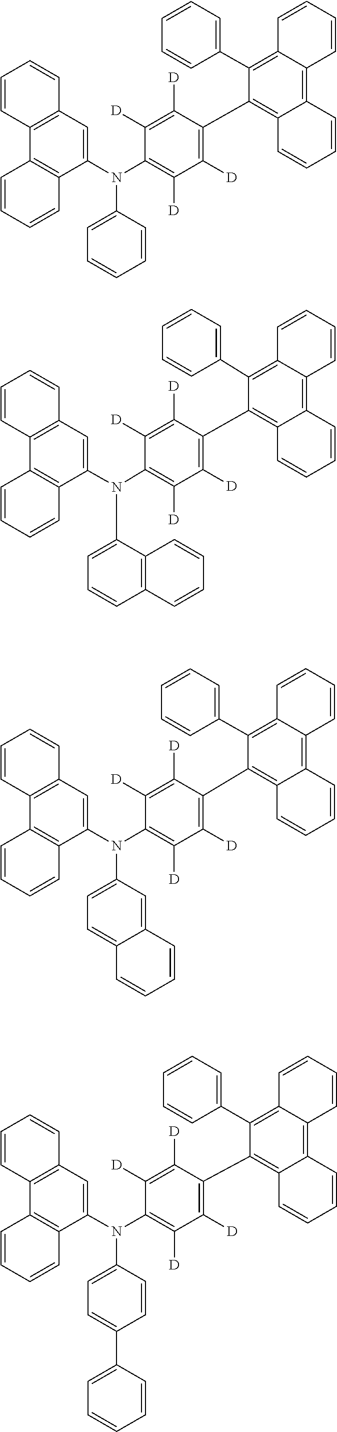 [SMILES: [2H]c1c([2H])c(N(c2ccccc2)c2cc3ccccc3c3ccccc23)c([2H])c([2H])c1-c1c(-c2ccccc2)c2ccccc2c2ccccc12 ; [2H]c1c([2H])c(N(c2cccc3ccccc23)c2cc3ccccc3c3ccccc23)c([2H])c([2H])c1-c1c(-c2ccccc2)c2ccccc2c2ccccc12 ; [2H]c1c([2H])c(N(c2ccc3ccccc3c2)c2cc3ccccc3c3ccccc23)c([2H])c([2H])c1-c1c(-c2ccccc2)c2ccccc2c2ccccc12 ; [2H]c1c([2H])c(N(c2ccc(-c3ccccc3)cc2)c2cc3ccccc3c3ccccc23)c([2H])c([2H])c1-c1c(-c2ccccc2)c2ccccc2c2ccccc12] 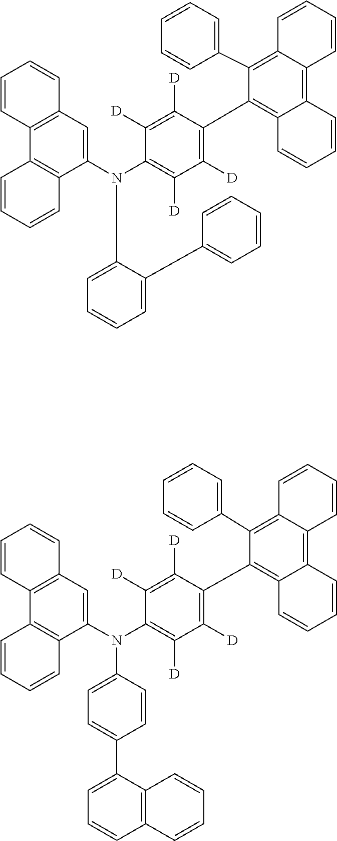 [SMILES: [2H]c1c([2H])c(N(c2ccccc2-c2ccccc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4ccccc34)cc2)c2cc3ccccc3c3ccccc23)c([2H])c([2H])c1-c1c(-c2ccccc2)c2ccccc2c2ccccc12] 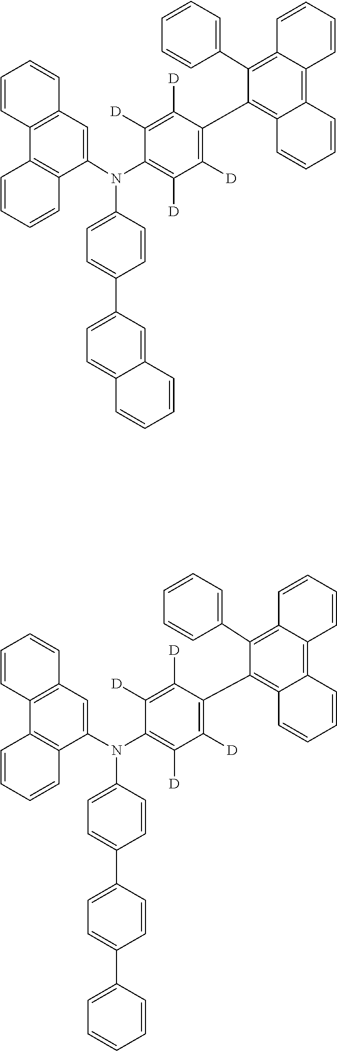 [SMILES: [2H]c1c([2H])c(N(c2ccc(-c3ccc4ccccc4c3)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1c(-c2ccccc2)c2ccccc2c2ccccc12] 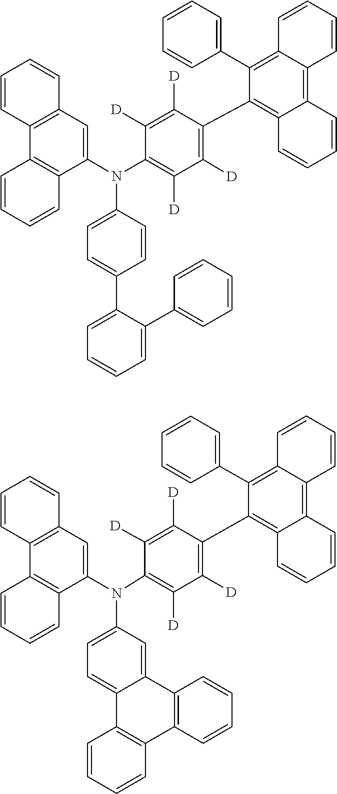 [SMILES: [2H]c1c([2H])c(N(c2ccc(-c3ccccc3-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccc3c4ccccc4c4ccccc4c3c2)c2cc3ccccc3c3ccccc23)c([2H])c([2H])c1-c1c(-c2ccccc2)c2ccccc2c2ccccc12] 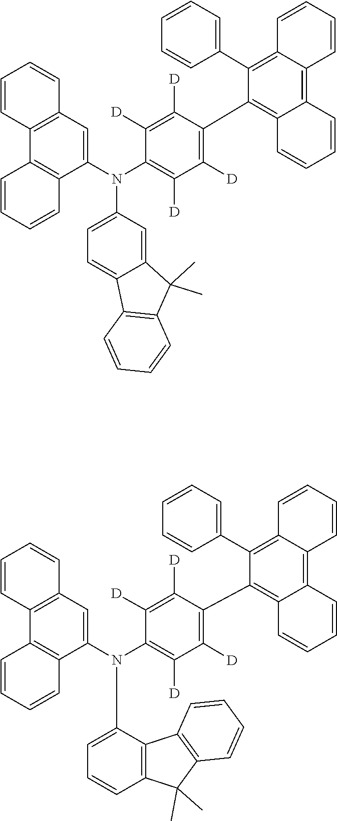 [SMILES: [2H]c1c([2H])c(N(c2ccc3c(c2)C(C)(C)c2ccccc2-3)c2cc3ccccc3c3ccccc23)c([2H])c([2H])c1-c1c(-c2ccccc2)c2ccccc2c2ccccc12 ; [2H]c1c([2H])c(N(c2cccc3c2-c2ccccc2C3(C)C)c2cc3ccccc3c3ccccc23)c([2H])c([2H])c1-c1c(-c2ccccc2)c2ccccc2c2ccccc12] 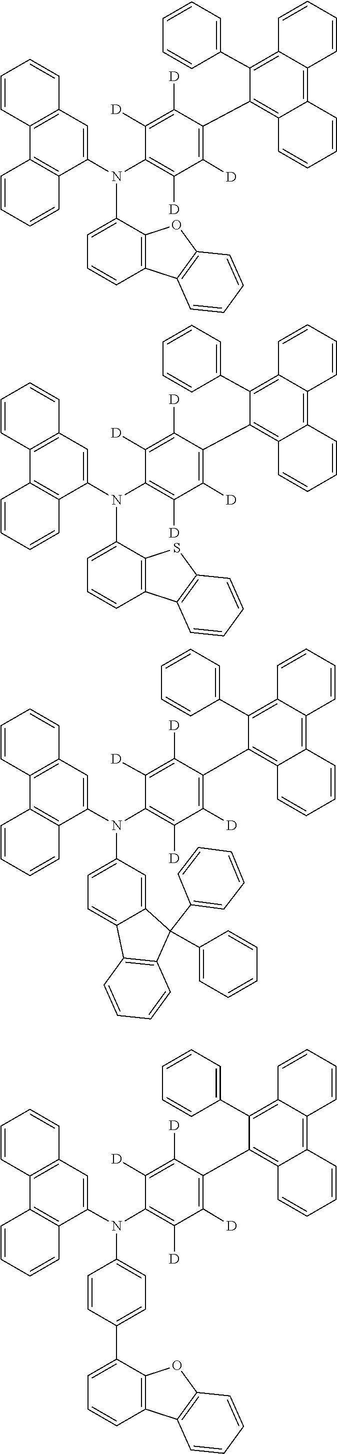 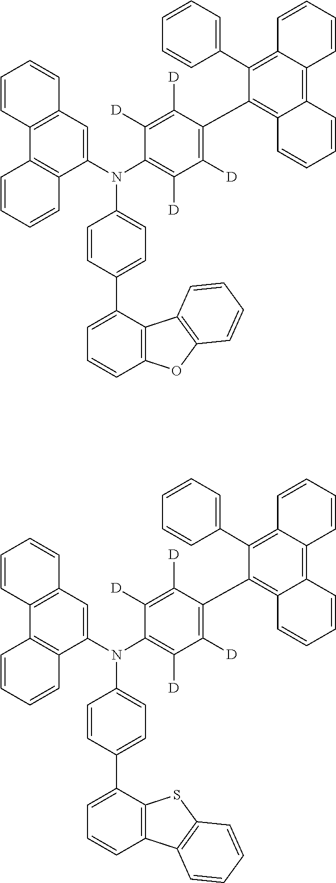 [SMILES: [2H]c1c([2H])c(N(c2ccc(-c3cccc4oc5ccccc5c34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3sc3ccccc34)cc2)c2cc3ccccc3c3ccccc23)c([2H])c([2H])c1-c1c(-c2ccccc2)c2ccccc2c2ccccc12] 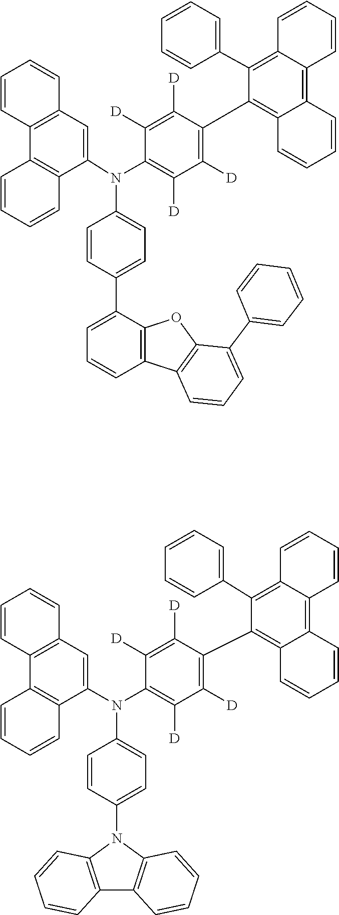 [SMILES: [2H]c1c([2H])c(N(c2ccc(-c3cccc4c3oc3c(-c5ccccc5)cccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1c(-c2ccccc2)c2ccccc2c2ccccc12] 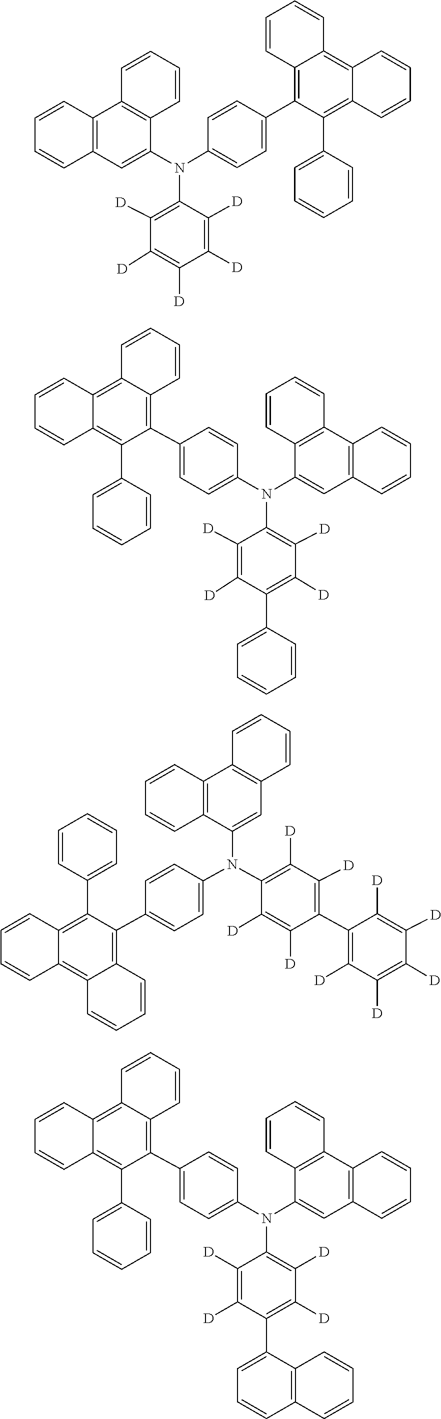 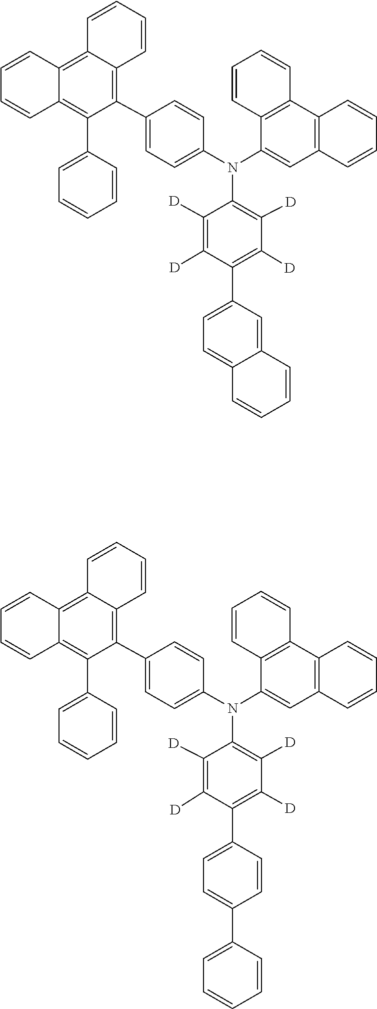 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc2ccccc2c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc(-c2ccccc2)cc1] 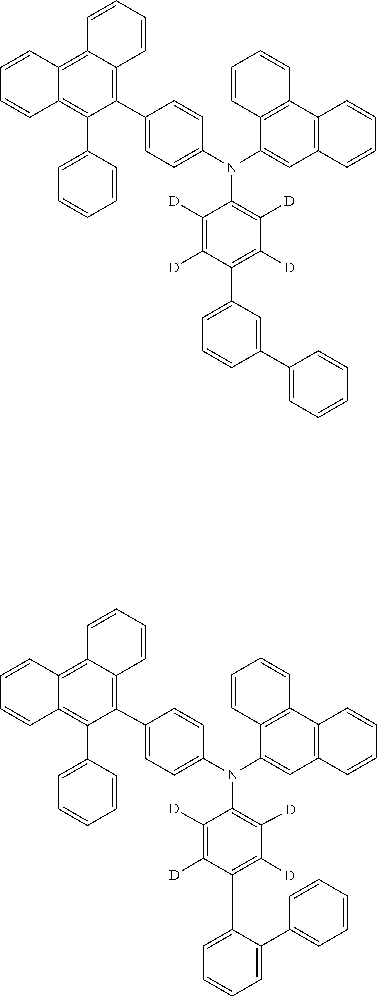 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc(-c2ccccc2)c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1-c1ccccc1] 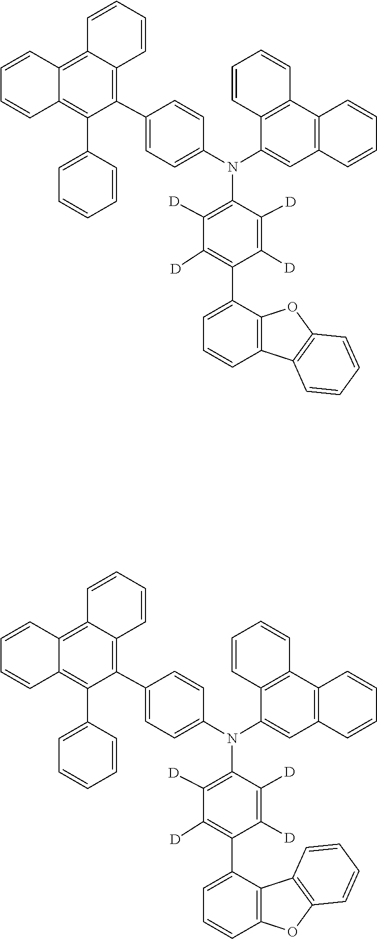 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1oc1ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2oc3ccccc3c12] 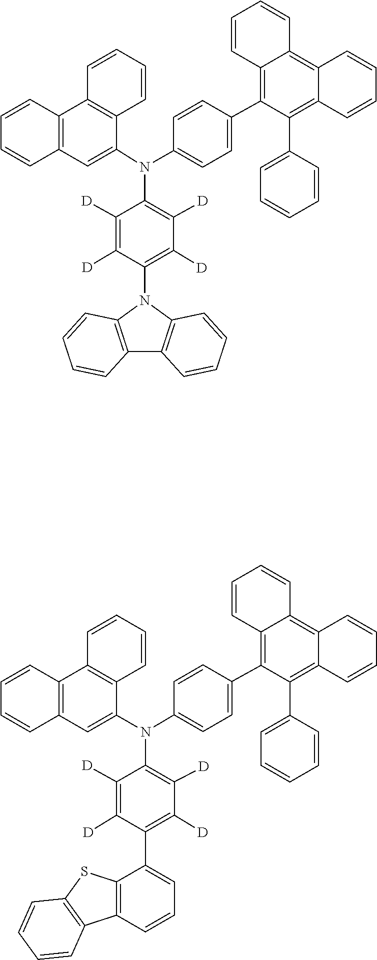 [SMILES: [2H]c1c([2H])c(-n2c3ccccc3c3ccccc32)c([2H])c([2H])c1N(c1ccc(-c2c(-c3ccccc3)c3ccccc3c3ccccc23)cc1)c1cc2ccccc2c2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1sc1ccccc12] 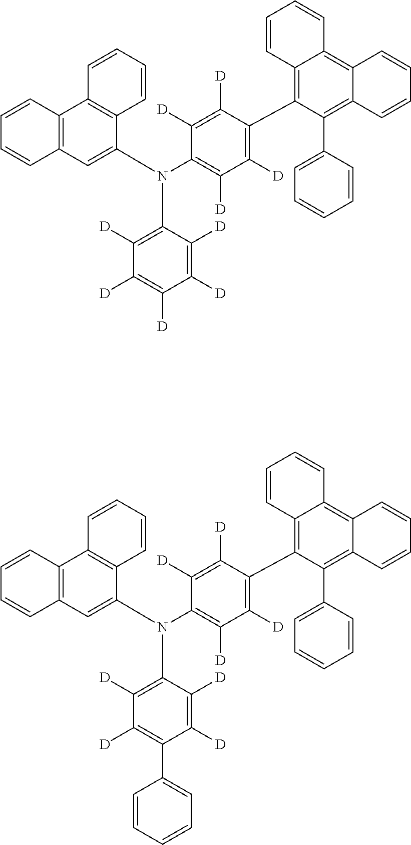 [SMILES: [2H]c1c([2H])c([2H])c(N(c2c([2H])c([2H])c(-c3c(-c4ccccc4)c4ccccc4c4ccccc34)c([2H])c2[2H])c2cc3ccccc3c3ccccc23)c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1] 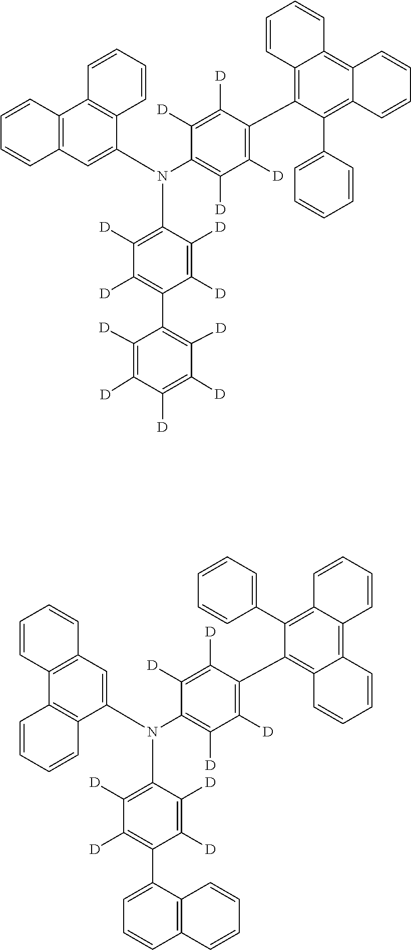 [SMILES: [2H]c1c([2H])c([2H])c(-c2c([2H])c([2H])c(N(c3c([2H])c([2H])c(-c4c(-c5ccccc5)c5ccccc5c5ccccc45)c([2H])c3[2H])c3cc4ccccc4c4ccccc34)c([2H])c2[2H])c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1cccc2ccccc12] 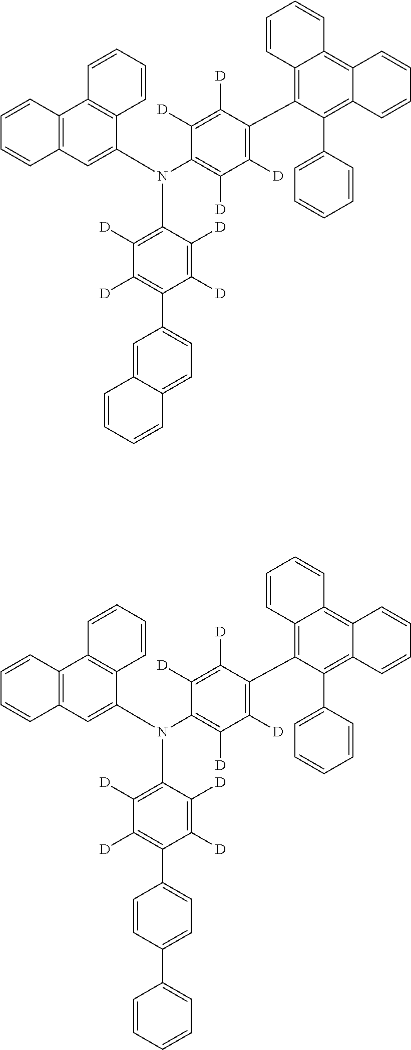 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc(-c2ccccc2)cc1] 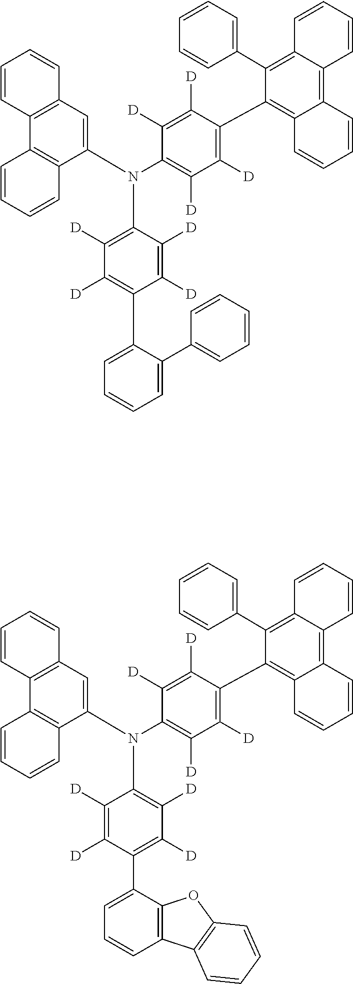 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1-c1ccccc1 ; [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1c(-c2ccccc2)c2ccccc2c2ccccc12] 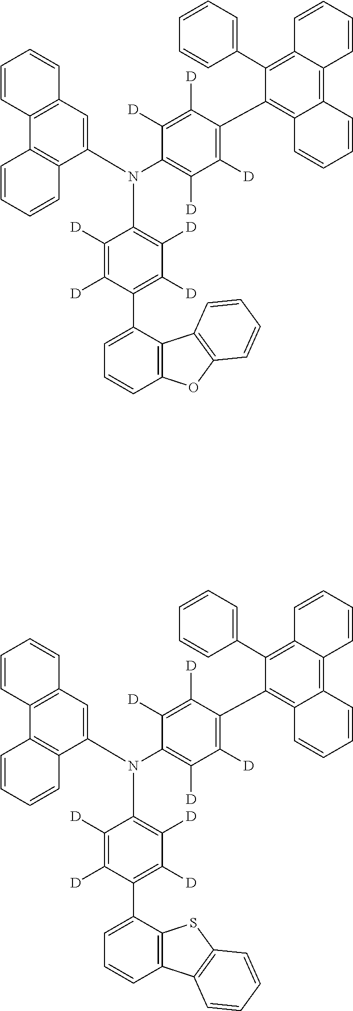 [SMILES: [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1c(-c2ccccc2)c2ccccc2c2ccccc12] 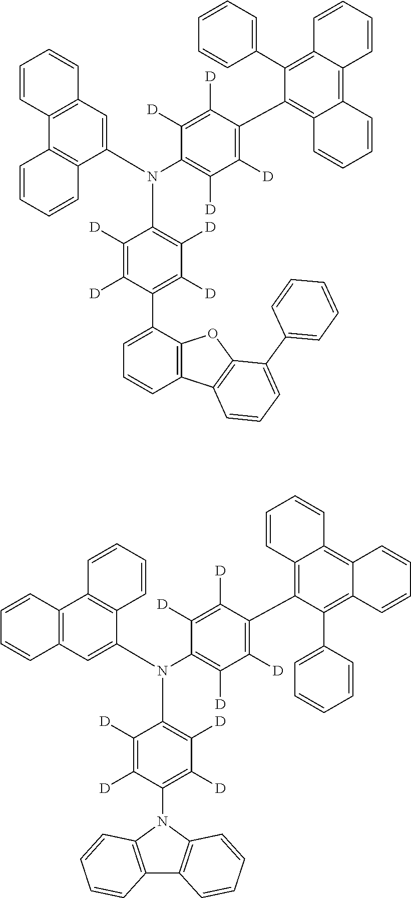 [SMILES: [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1c(-c2ccccc2)c2ccccc2c2ccccc12] 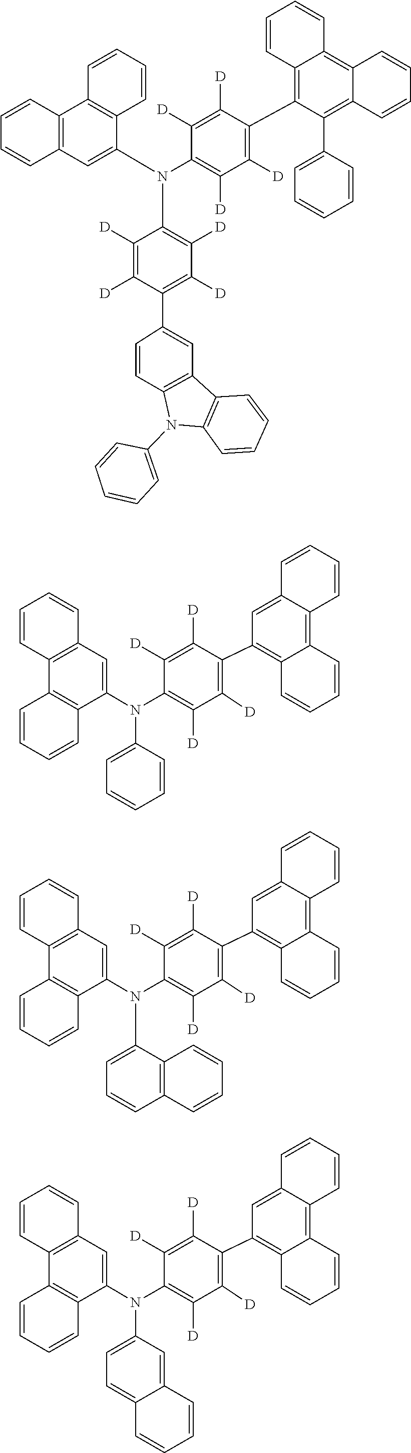 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2c(c1)c1ccccc1n2-c1ccccc1 ; [2H]c1c([2H])c(N(c2ccccc2)c2cc3ccccc3c3ccccc23)c([2H])c([2H])c1-c1cc2ccccc2c2ccccc12 ; [2H]c1c([2H])c(N(c2cccc3ccccc23)c2cc3ccccc3c3ccccc23)c([2H])c([2H])c1-c1cc2ccccc2c2ccccc12 ; [2H]c1c([2H])c(N(c2ccc3ccccc3c2)c2cc3ccccc3c3ccccc23)c([2H])c([2H])c1-c1cc2ccccc2c2ccccc12] 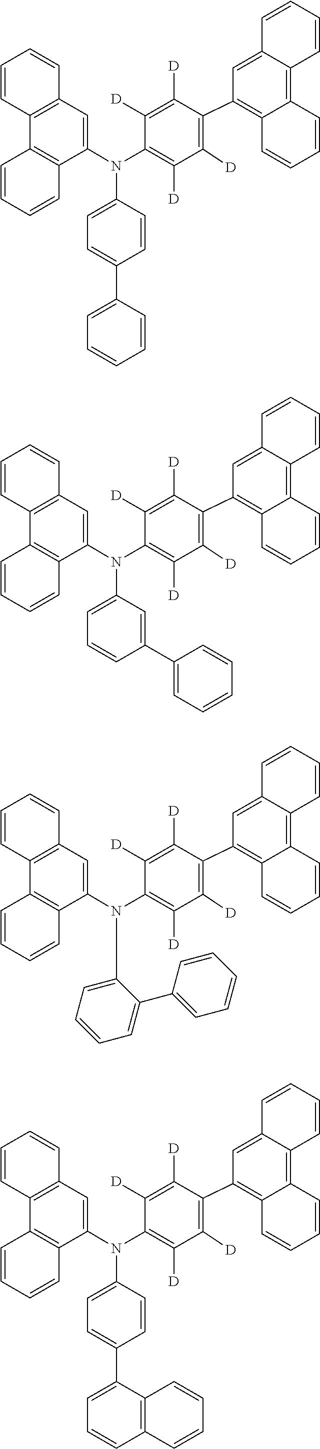 [SMILES: [2H]c1c([2H])c(N(c2ccc(-c3ccccc3)cc2)c2cc3ccccc3c3ccccc23)c([2H])c([2H])c1-c1cc2ccccc2c2ccccc12 ; [2H]c1c([2H])c(N(c2cccc(-c3ccccc3)c2)c2cc3ccccc3c3ccccc23)c([2H])c([2H])c1-c1cc2ccccc2c2ccccc12 ; [2H]c1c([2H])c(N(c2ccccc2-c2ccccc2)c2cc3ccccc3c3ccccc23)c([2H])c([2H])c1-c1cc2ccccc2c2ccccc12 ; [2H]c1c([2H])c(N(c2ccc(-c3cccc4ccccc34)cc2)c2cc3ccccc3c3ccccc23)c([2H])c([2H])c1-c1cc2ccccc2c2ccccc12] 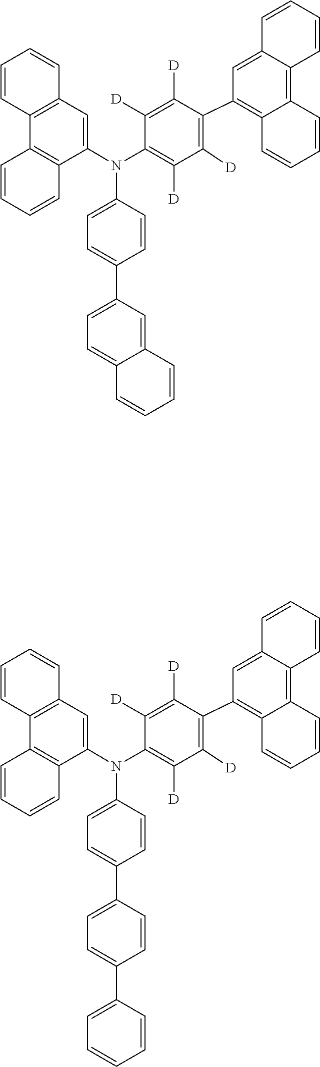 [SMILES: [2H]c1c([2H])c(N(c2ccc(-c3ccc4ccccc4c3)cc2)c2cc3ccccc3c3ccccc23)c([2H])c([2H])c1-c1cc2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1cc2ccccc2c2ccccc12] 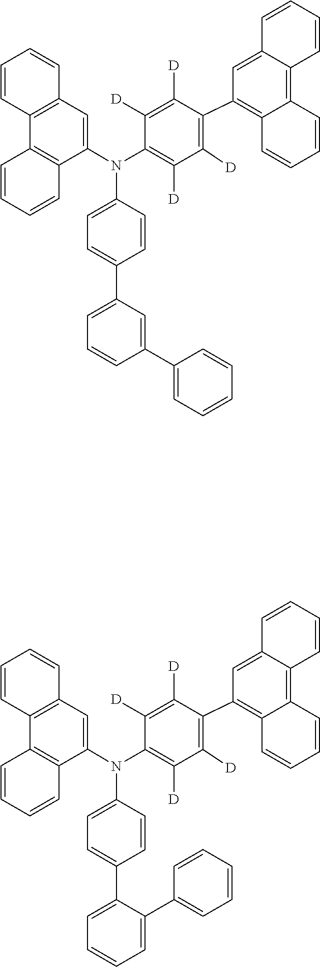 [SMILES: [2H]c1c([2H])c(N(c2ccc(-c3cccc(-c4ccccc4)c3)cc2)c2cc3ccccc3c3ccccc23)c([2H])c([2H])c1-c1cc2ccccc2c2ccccc12 ; [2H]c1c([2H])c(N(c2ccc(-c3ccccc3-c3ccccc3)cc2)c2cc3ccccc3c3ccccc23)c([2H])c([2H])c1-c1cc2ccccc2c2ccccc12] 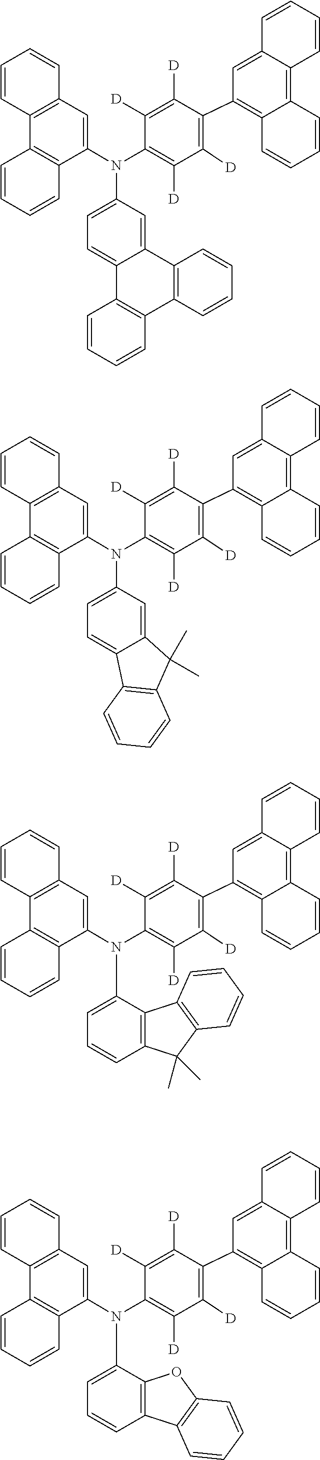 [SMILES: [2H]c1c([2H])c(N(c2ccc3c4ccccc4c4ccccc4c3c2)c2cc3ccccc3c3ccccc23)c([2H])c([2H])c1-c1cc2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(C)C)c2cc3ccccc3c3ccccc23)c([2H])c([2H])c1-c1cc2ccccc2c2ccccc12 ; [2H]c1c([2H])c(N(c2cc3ccccc3c3ccccc23)c2cccc3c2oc2ccccc23)c([2H])c([2H])c1-c1cc2ccccc2c2ccccc12] 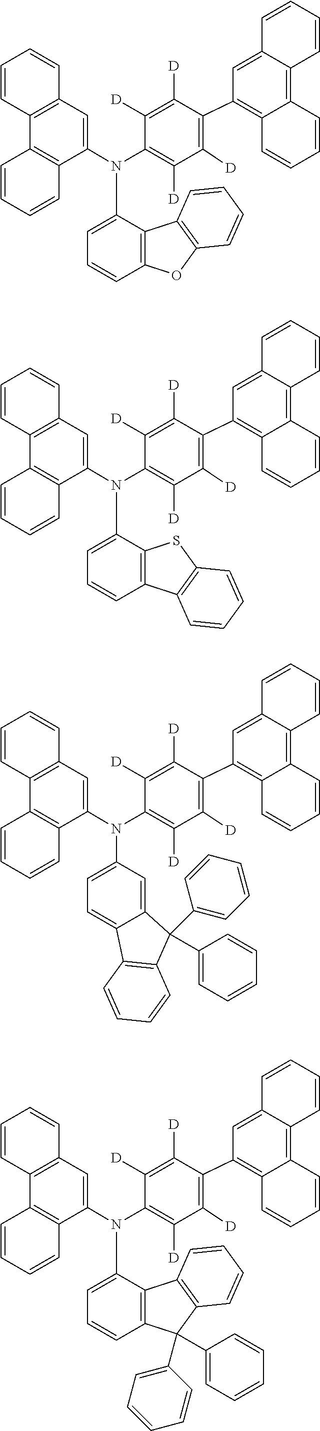 [SMILES: [2H]c1c([2H])c(N(c2cc3ccccc3c3ccccc23)c2cccc3oc4ccccc4c23)c([2H])c([2H])c1-c1cc2ccccc2c2ccccc12 ; [2H]c1c([2H])c(N(c2cc3ccccc3c3ccccc23)c2cccc3c2sc2ccccc23)c([2H])c([2H])c1-c1cc2ccccc2c2ccccc12 ; [2H]c1c([2H])c(N(c2ccc3c(c2)C(c2ccccc2)(c2ccccc2)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(c2ccccc2)c2ccccc2)c2cc3ccccc3c3ccccc23)c([2H])c([2H])c1-c1cc2ccccc2c2ccccc12] 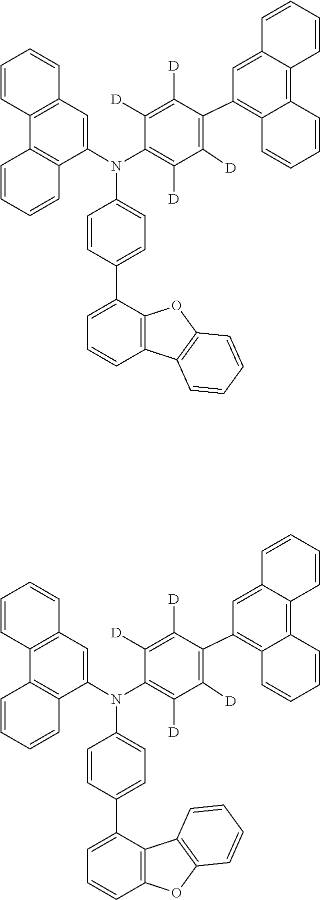 [SMILES: [2H]c1c([2H])c(N(c2ccc(-c3cccc4c3oc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1cc2ccccc2c2ccccc12] 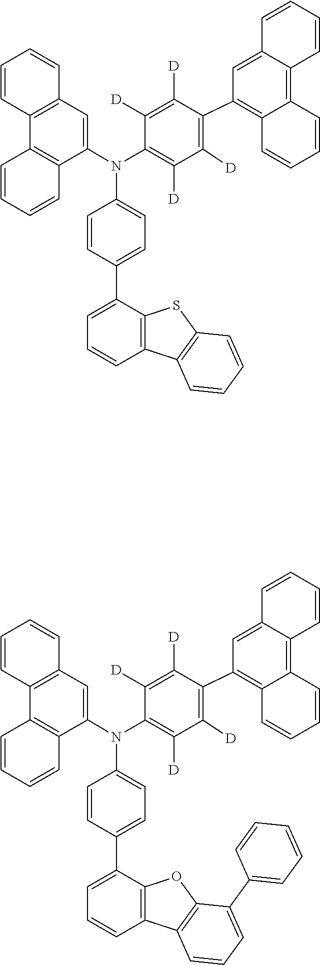 [SMILES: [2H]c1c([2H])c(N(c2ccc(-c3cccc4c3sc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1cc2ccccc2c2ccccc12] 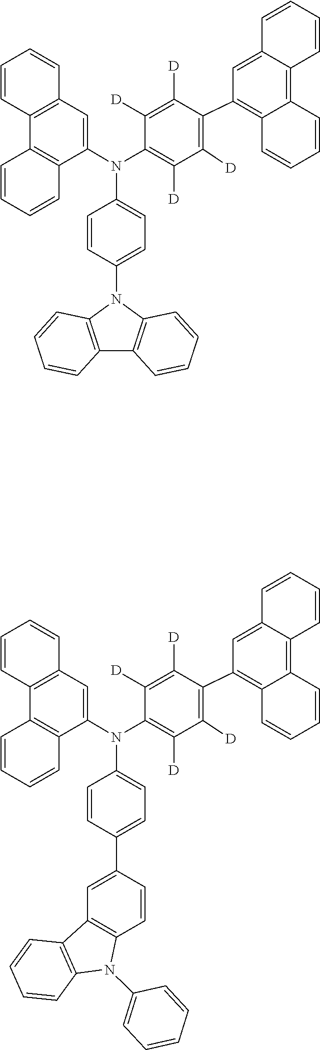 [SMILES: [2H]c1c([2H])c(N(c2ccc(-n3c4ccccc4c4ccccc43)cc2)c2cc3ccccc3c3ccccc23)c([2H])c([2H])c1-c1cc2ccccc2c2ccccc12 ; [2H]c1c([2H])c(N(c2ccc(-c3ccc4c(c3)c3ccccc3n4-c3ccccc3)cc2)c2cc3ccccc3c3ccccc23)c([2H])c([2H])c1-c1cc2ccccc2c2ccccc12] 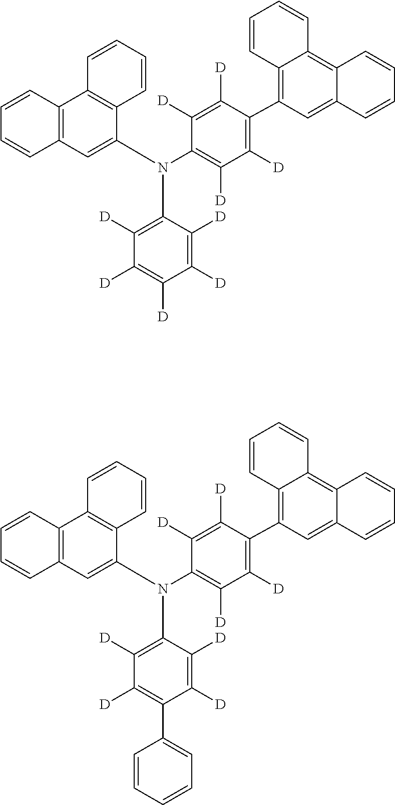 [SMILES: [2H]c1c([2H])c([2H])c(N(c2c([2H])c([2H])c(-c3cc4ccccc4c4ccccc34)c([2H])c2[2H])c2cc3ccccc3c3ccccc23)c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1] 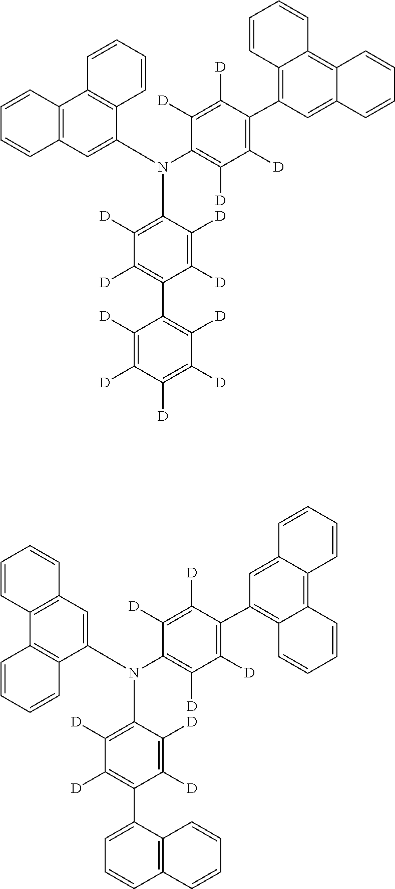 [SMILES: [2H]c1c([2H])c([2H])c(-c2c([2H])c([2H])c(N(c3c([2H])c([2H])c(-c4cc5ccccc5c5ccccc45)c([2H])c3[2H])c3cc4ccccc4c4ccccc34)c([2H])c2[2H])c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc2ccccc12] 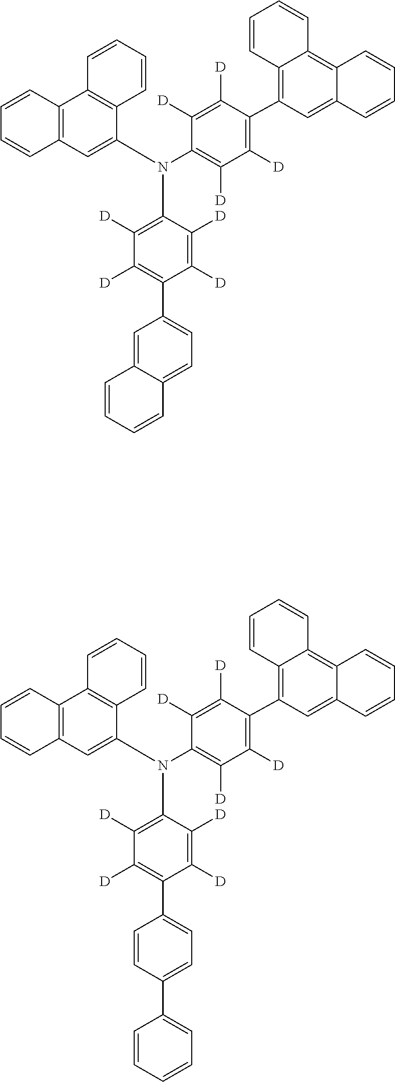 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc(-c2ccccc2)cc1] 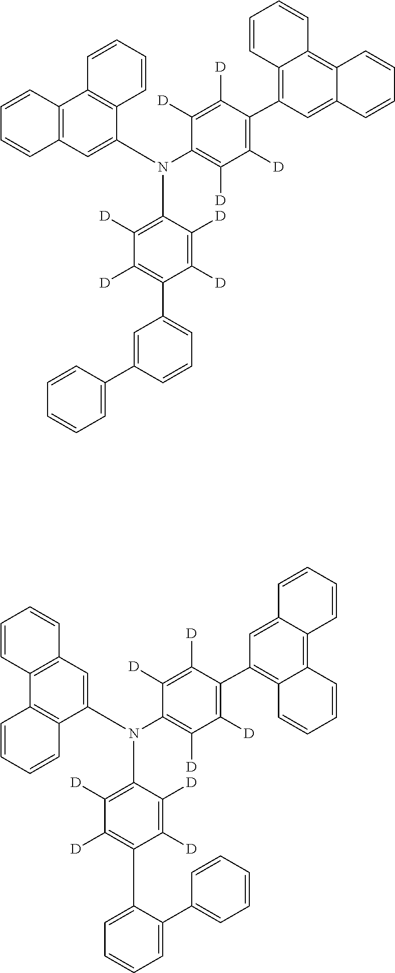 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc(-c2ccccc2)c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1-c1ccccc1] 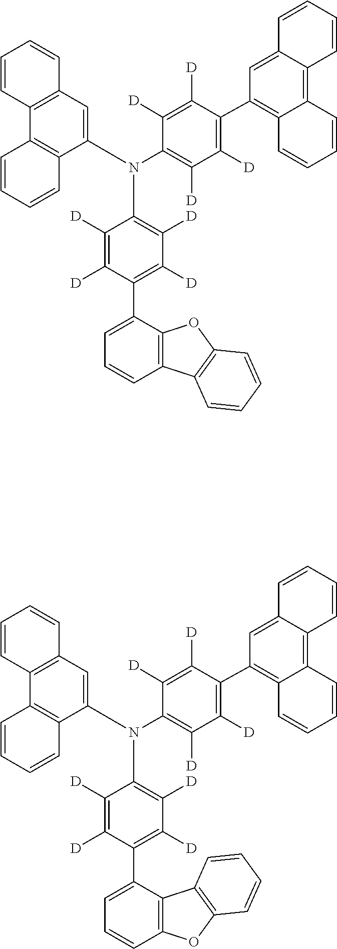 [SMILES: [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1cc2ccccc2c2ccccc12] 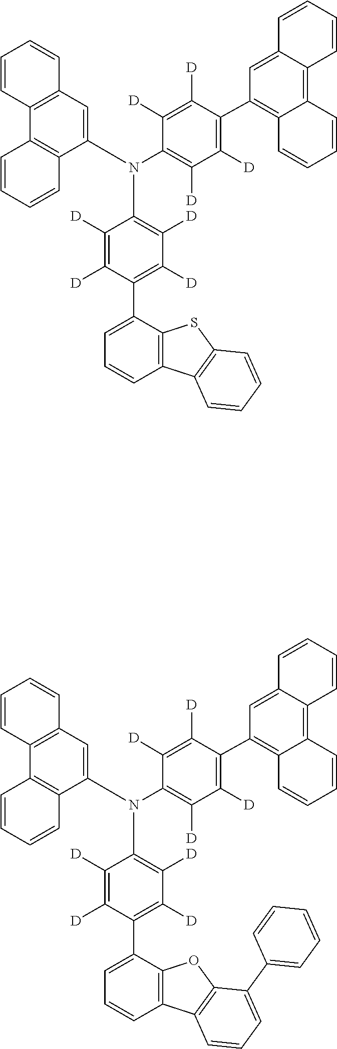 [SMILES: [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1cc2ccccc2c2ccccc12] 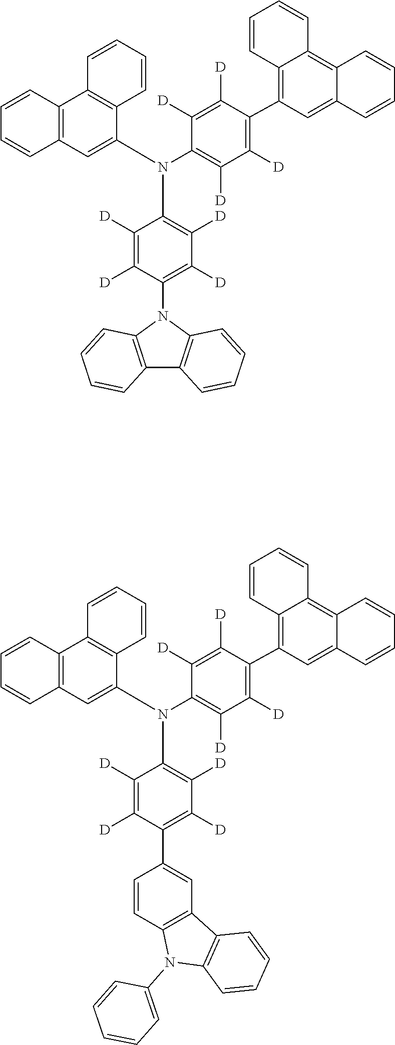 [SMILES: [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2c(c1)c1ccccc1n2-c1ccccc1] 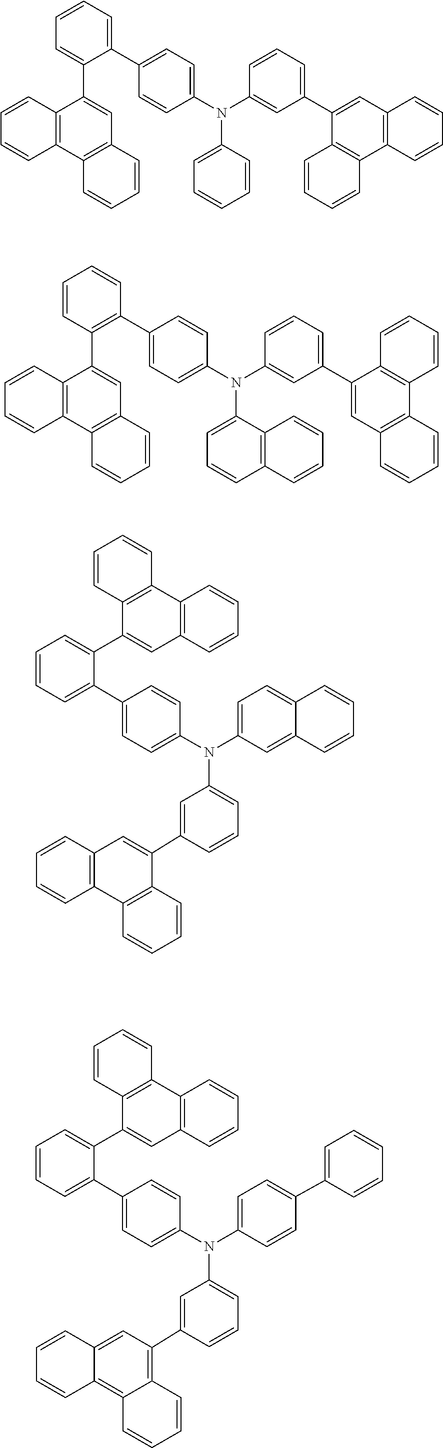 [SMILES: c1ccc(N(c2ccc(-c3ccccc3-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3ccccc3-c3cc4ccccc4c4ccccc34)cc2)c2cccc3ccccc23)c1 ; c1cc(-c2cc3ccccc3c3ccccc23)cc(N(c2ccc(-c3ccccc3-c3cc4ccccc4c4ccccc34)cc2)c2ccc3ccccc3c2)c1 ; c1ccc(-c2ccc(N(c3ccc(-c4ccccc4-c4cc5ccccc5c5ccccc45)cc3)c3cccc(-c4cc5ccccc5c5ccccc45)c3)cc2)cc1] 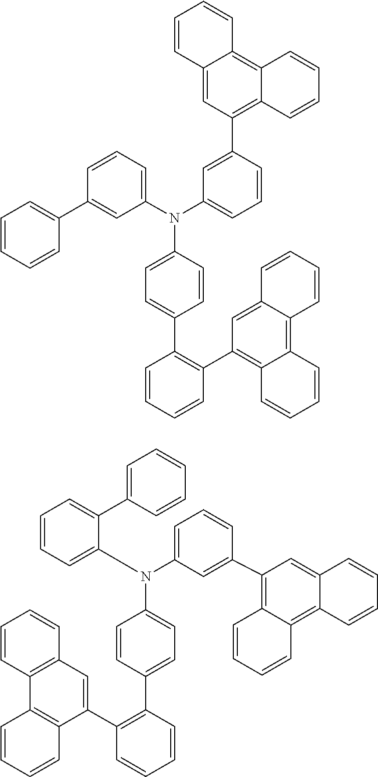 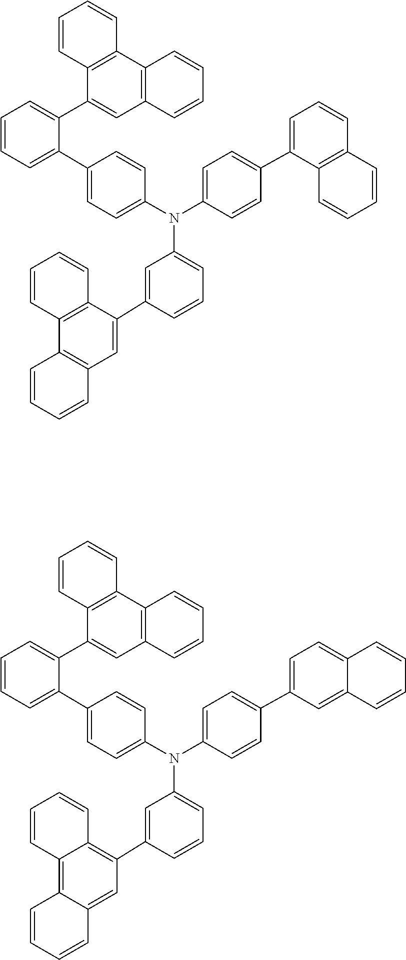 [SMILES: c1cc(-c2cc3ccccc3c3ccccc23)cc(N(c2ccc(-c3ccccc3-c3cc4ccccc4c4ccccc34)cc2)c2ccc(-c3cccc4ccccc34)cc2)c1 ; c1cc(-c2cc3ccccc3c3ccccc23)cc(N(c2ccc(-c3ccc4ccccc4c3)cc2)c2ccc(-c3ccccc3-c3cc4ccccc4c4ccccc34)cc2)c1] 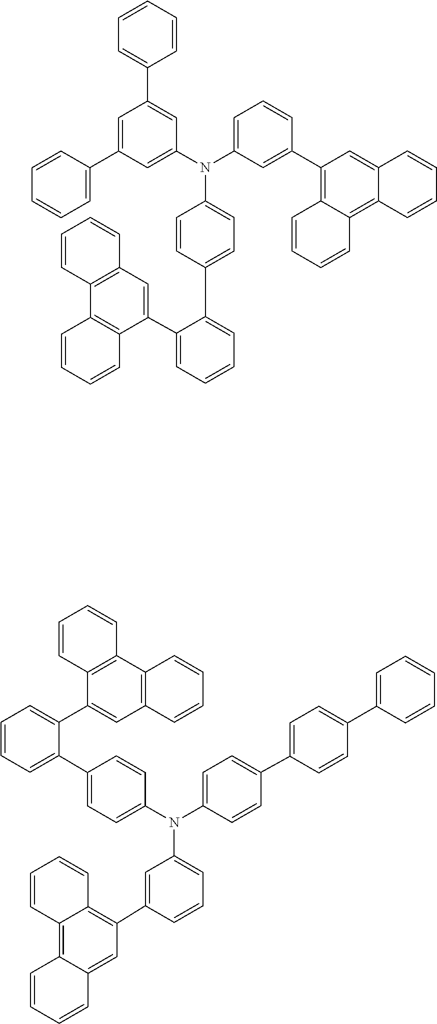 [SMILES: c1ccc(-c2cc(-c3ccccc3)cc(N(c3ccc(-c4ccccc4-c4cc5ccccc5c5ccccc45)cc3)c3cccc(-c4cc5ccccc5c5ccccc45)c3)c2)cc1 ; c1ccc(-c2ccc(-c3ccc(N(c4ccc(-c5ccccc5-c5cc6ccccc6c6ccccc56)cc4)c4cccc(-c5cc6ccccc6c6ccccc56)c4)cc3)cc2)cc1] 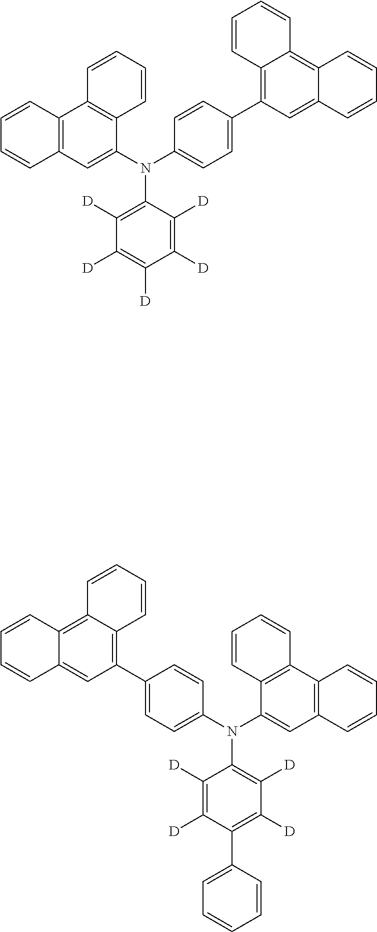 [SMILES: [2H]c1c([2H])c([2H])c(N(c2ccc(-c3cc4ccccc4c4ccccc34)cc2)c2cc3ccccc3c3ccccc23)c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1ccccc1] 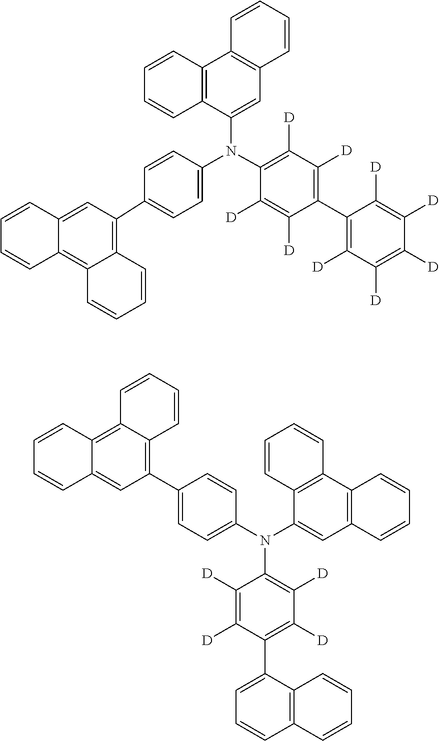 [SMILES: [2H]c1c([2H])c([2H])c(-c2c([2H])c([2H])c(N(c3ccc(-c4cc5ccccc5c5ccccc45)cc3)c3cc4ccccc4c4ccccc34)c([2H])c2[2H])c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1cccc2ccccc12] 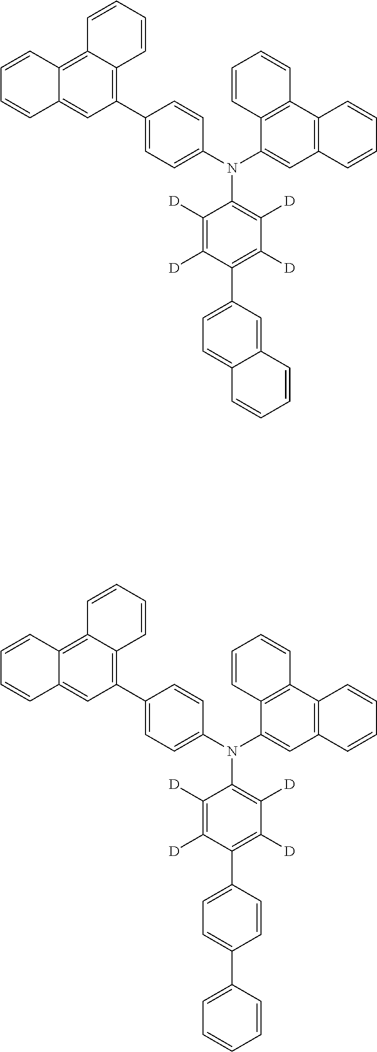 [SMILES: [2H]c1c([2H])c(N(c2ccc(-c3cc4ccccc4c4ccccc34)cc2)c2cc3ccccc3c3ccccc23)c([2H])c([2H])c1-c1ccc2ccccc2c1 ; [2H]c1c([2H])c(N(c2ccc(-c3cc4ccccc4c4ccccc34)cc2)c2cc3ccccc3c3ccccc23)c([2H])c([2H])c1-c1ccc(-c2ccccc2)cc1] 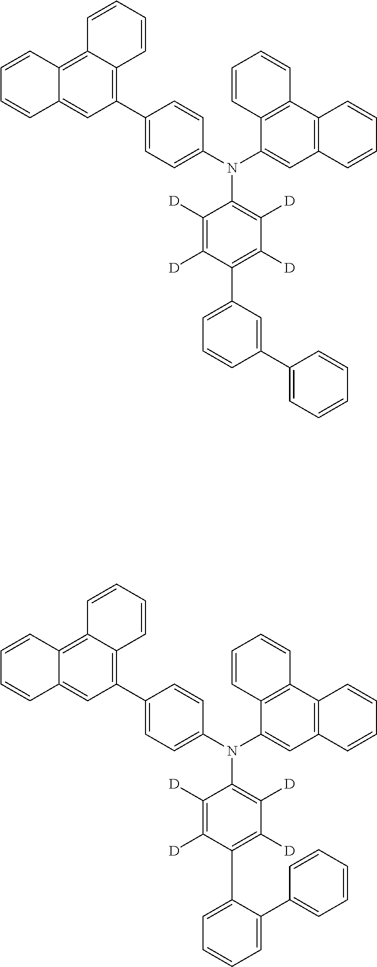 [SMILES: [2H]c1c([2H])c(N(c2ccc(-c3cc4ccccc4c4ccccc34)cc2)c2cc3ccccc3c3ccccc23)c([2H])c([2H])c1-c1cccc(-c2ccccc2)c1 ; [2H]c1c([2H])c(N(c2ccc(-c3cc4ccccc4c4ccccc34)cc2)c2cc3ccccc3c3ccccc23)c([2H])c([2H])c1-c1ccccc1-c1ccccc1] 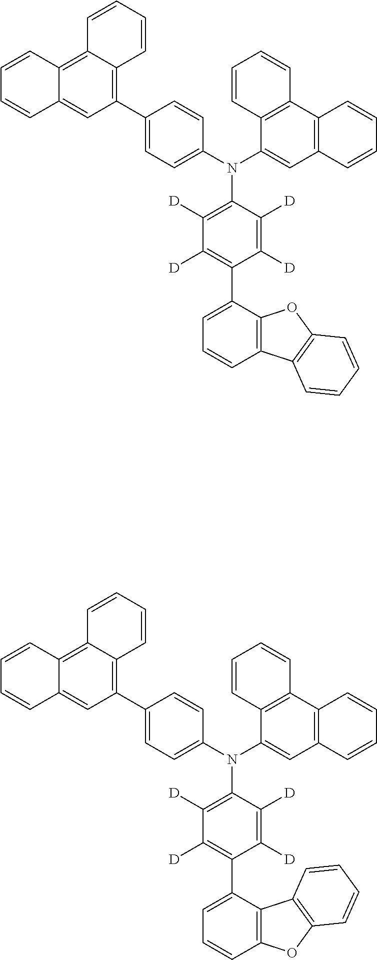 [SMILES: [2H]c1c([2H])c(N(c2ccc(-c3cc4ccccc4c4ccccc34)cc2)c2cc3ccccc3c3ccccc23)c([2H])c([2H])c1-c1cccc2c1oc1ccccc12 ; [2H]c1c([2H])c(N(c2ccc(-c3cc4ccccc4c4ccccc34)cc2)c2cc3ccccc3c3ccccc23)c([2H])c([2H])c1-c1cccc2oc3ccccc3c12] 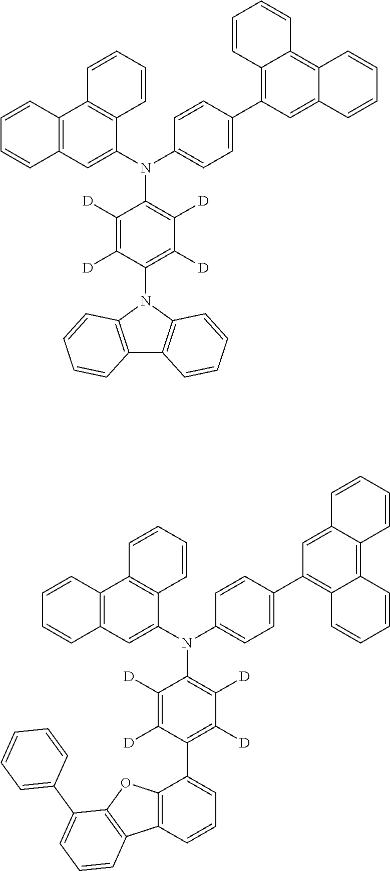 [SMILES: [2H]c1c([2H])c(-n2c3ccccc3c3ccccc32)c([2H])c([2H])c1N(c1ccc(-c2cc3ccccc3c3ccccc23)cc1)c1cc2ccccc2c2ccccc12 ; [2H]c1c([2H])c(N(c2ccc(-c3cc4ccccc4c4ccccc34)cc2)c2cc3ccccc3c3ccccc23)c([2H])c([2H])c1-c1cccc2c1oc1c(-c3ccccc3)cccc12] 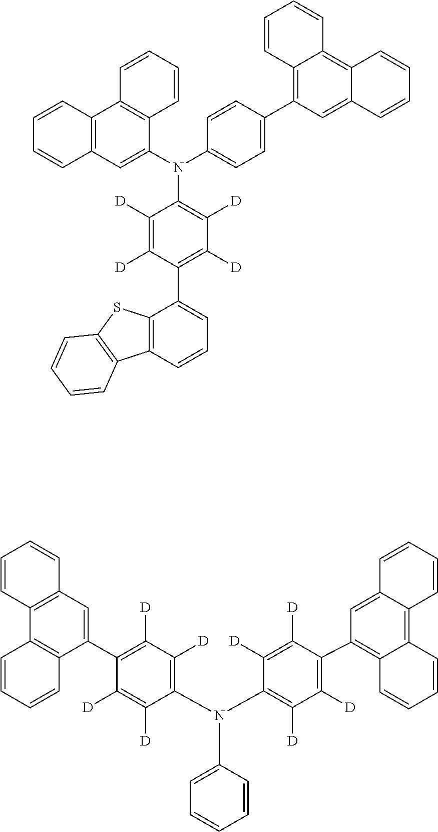 [SMILES: [2H]c1c([2H])c(N(c2ccc(-c3cc4ccccc4c4ccccc34)cc2)c2cc3ccccc3c3ccccc23)c([2H])c([2H])c1-c1cccc2c1sc1ccccc12 ; [2H]c1c([2H])c(N(c2ccccc2)c2c([2H])c([2H])c(-c3cc4ccccc4c4ccccc34)c([2H])c2[2H])c([2H])c([2H])c1-c1cc2ccccc2c2ccccc12] 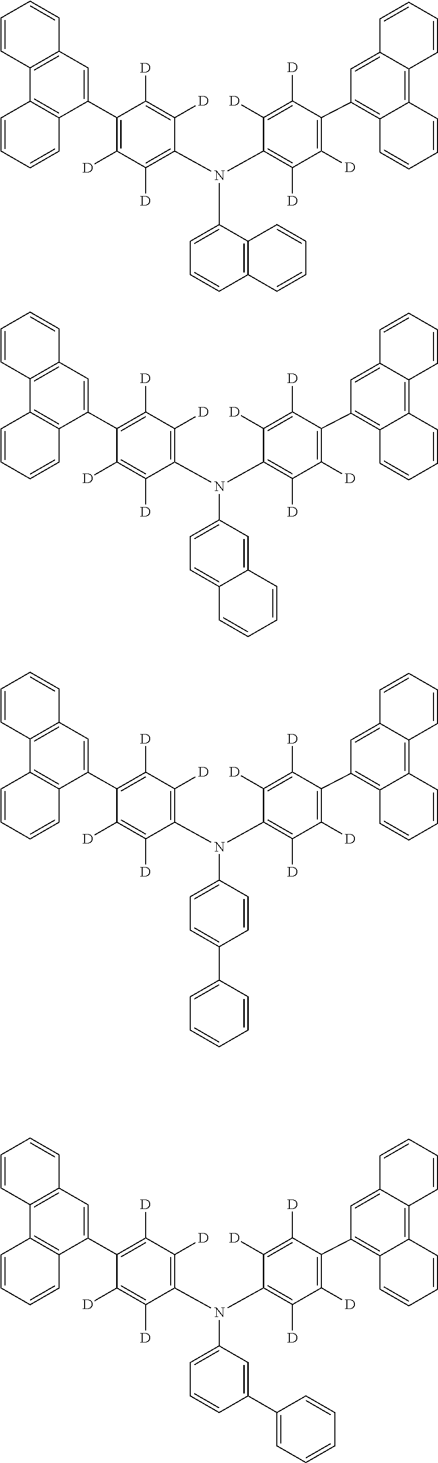 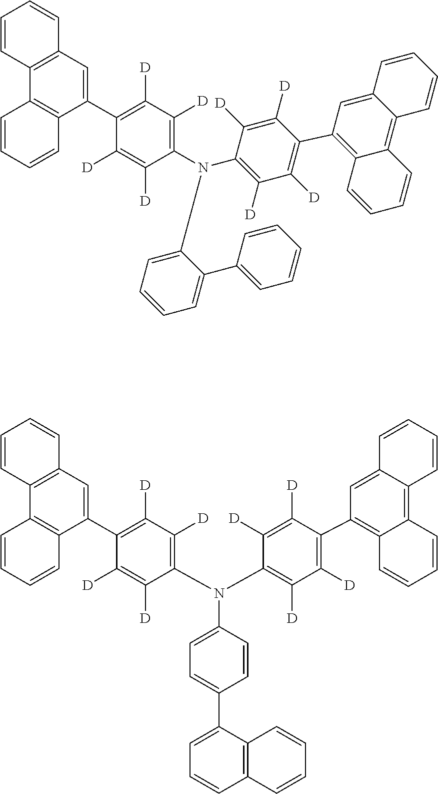 [SMILES: [2H]c1c([2H])c(N(c2ccccc2-c2ccccc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc4ccccc34)cc2)c2c([2H])c([2H])c(-c3cc4ccccc4c4ccccc34)c([2H])c2[2H])c([2H])c([2H])c1-c1cc2ccccc2c2ccccc12] 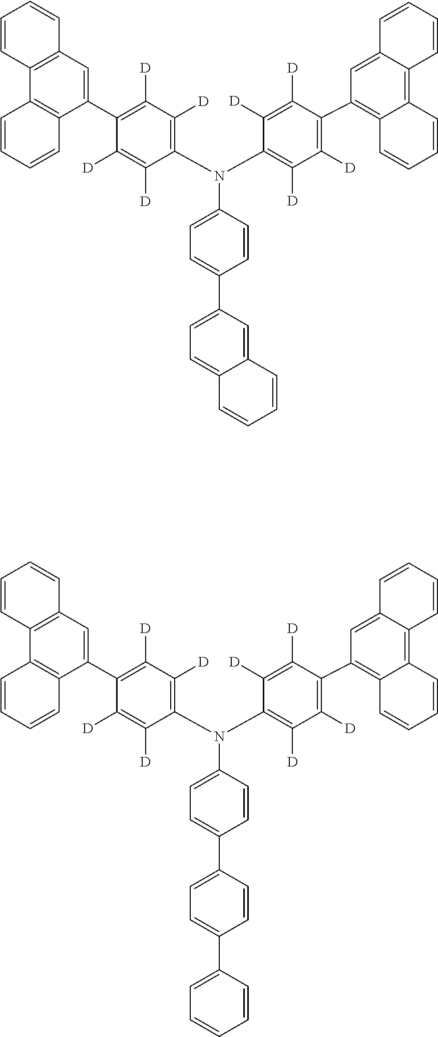 [SMILES: [2H]c1c([2H])c(N(c2ccc(-c3ccc4ccccc4c3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccc(-c4ccccc4)cc3)cc2)c2c([2H])c([2H])c(-c3cc4ccccc4c4ccccc34)c([2H])c2[2H])c([2H])c([2H])c1-c1cc2ccccc2c2ccccc12] 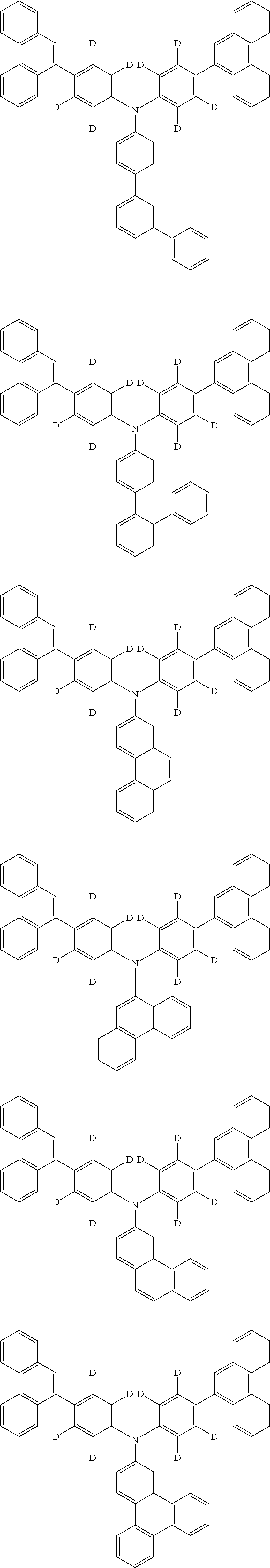 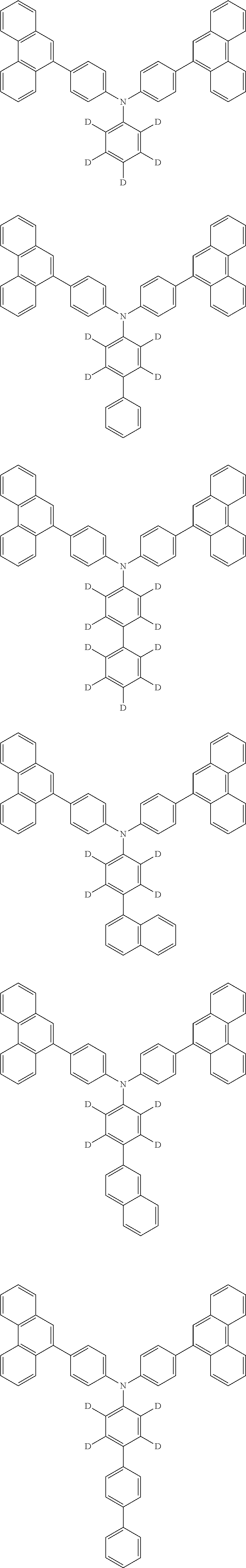 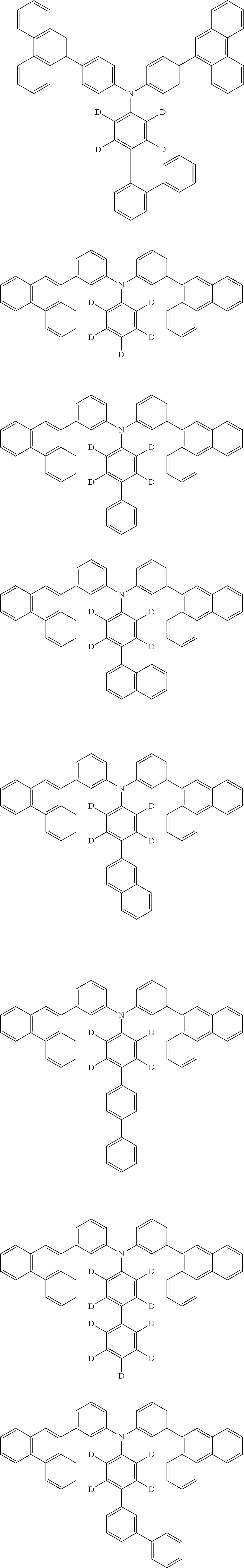 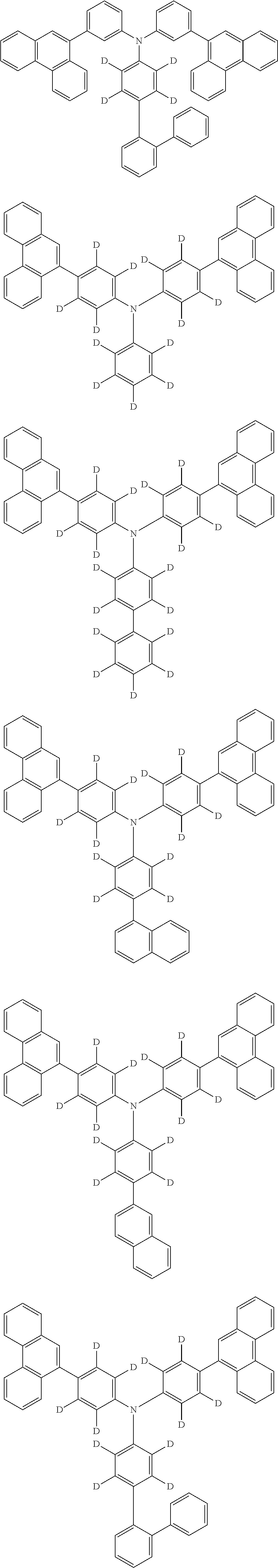 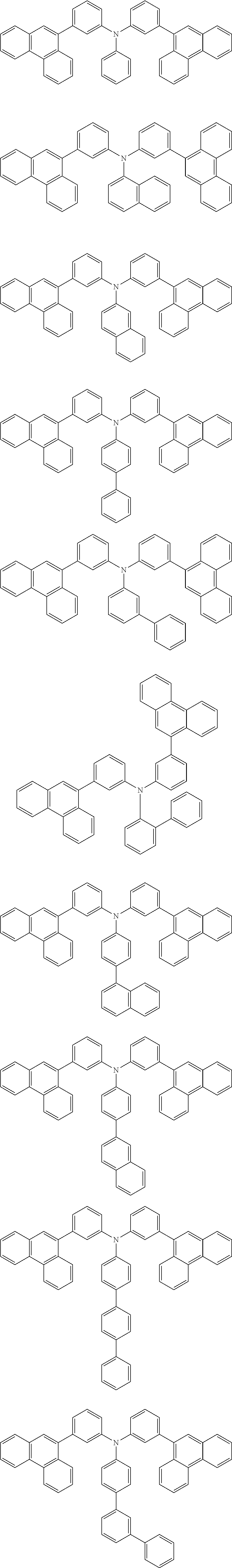 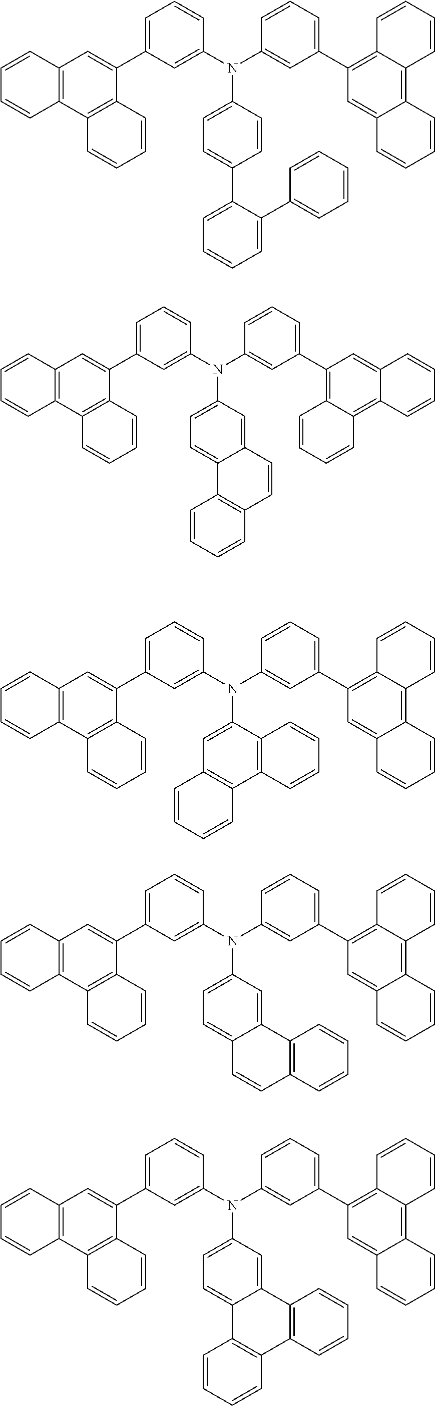 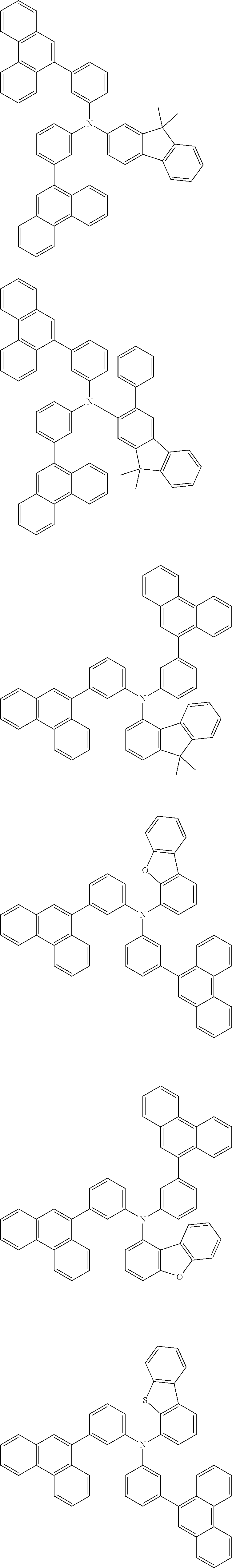 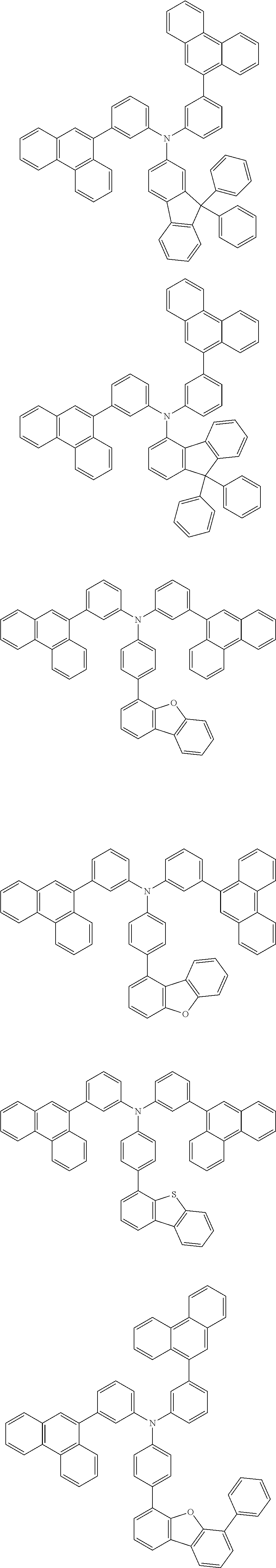 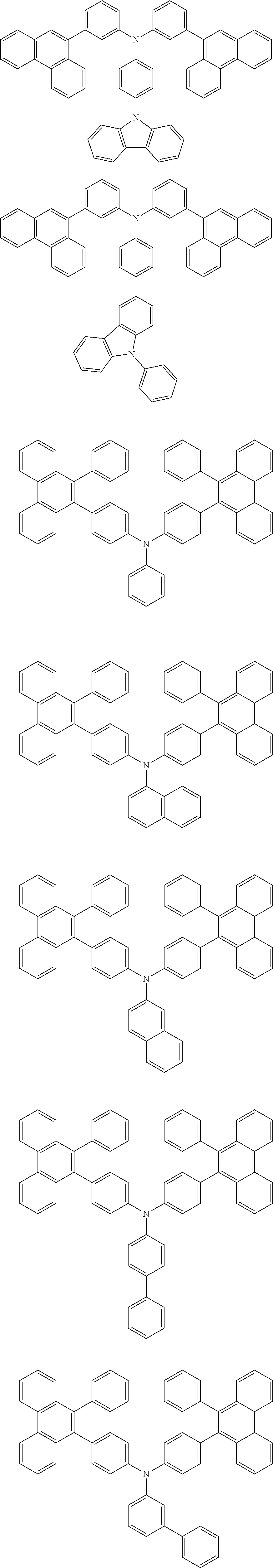 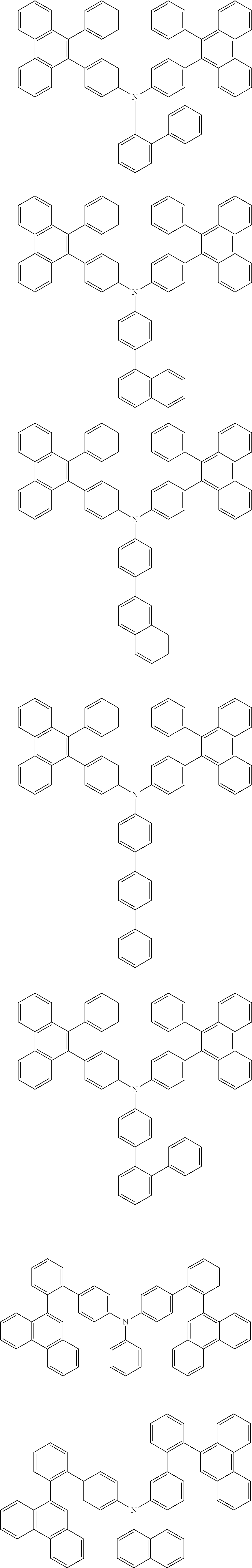 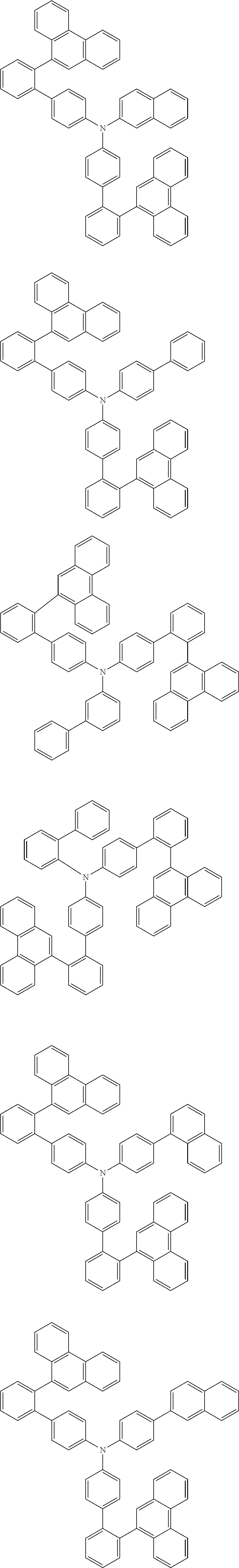 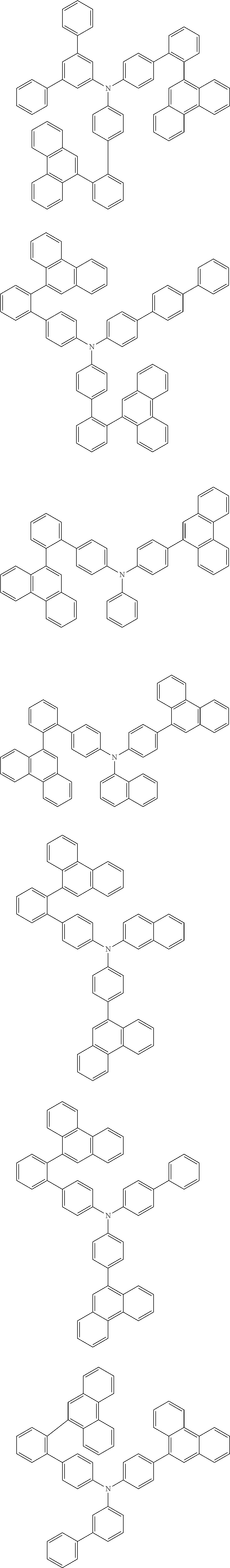 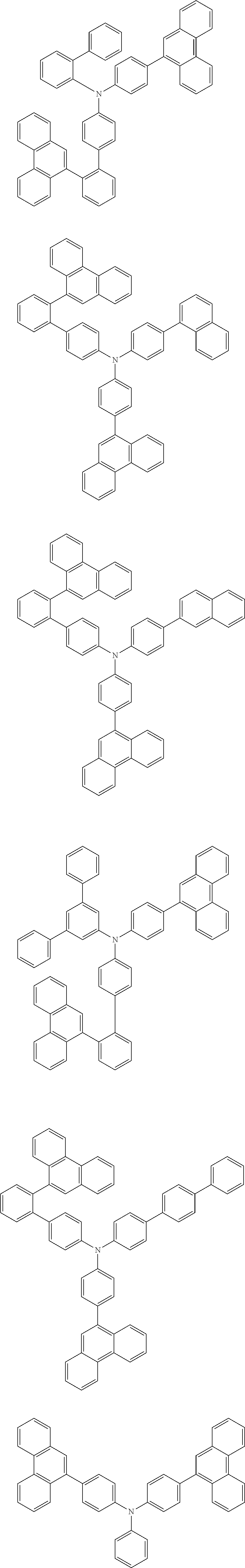 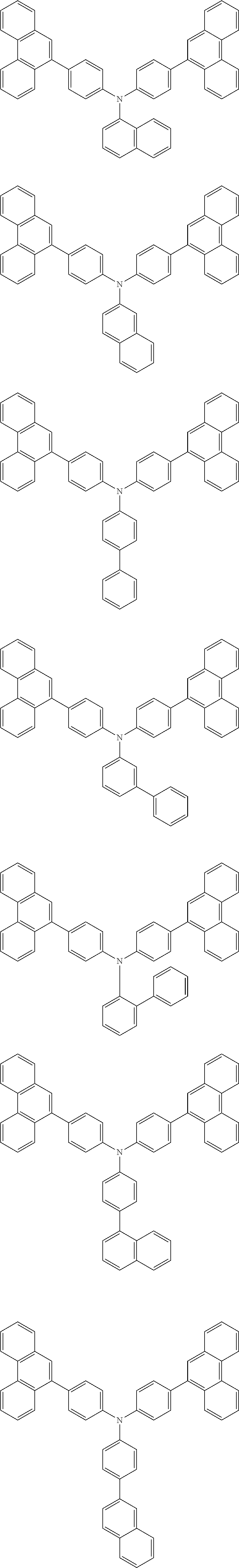 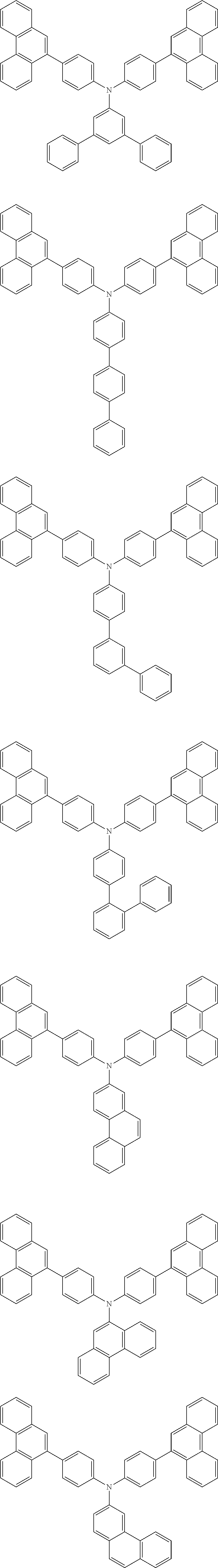 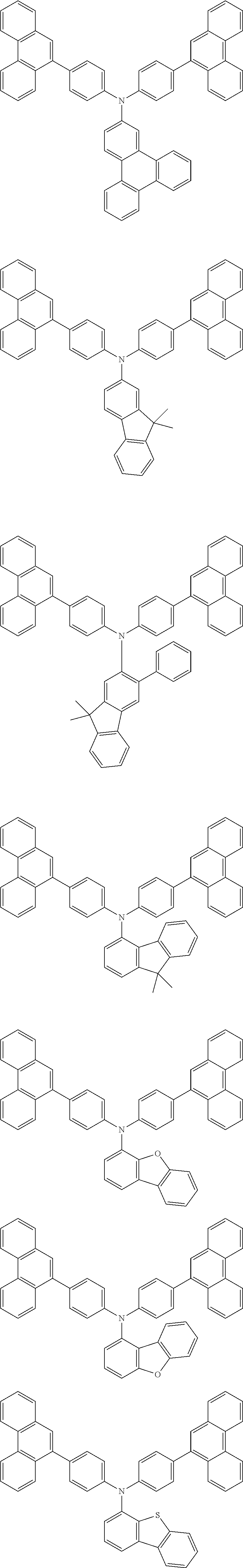 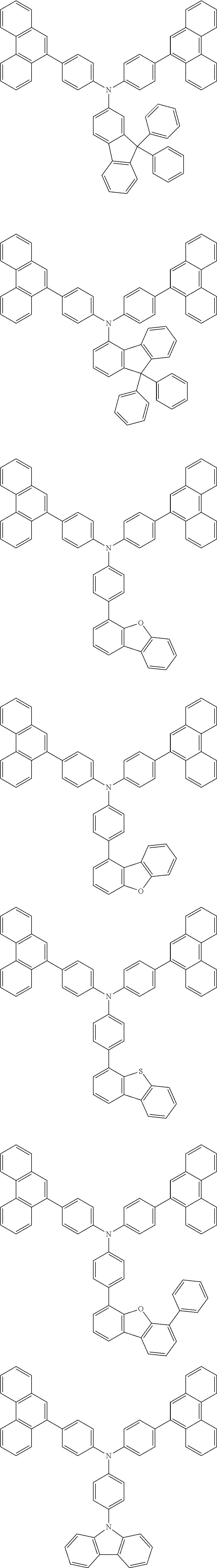 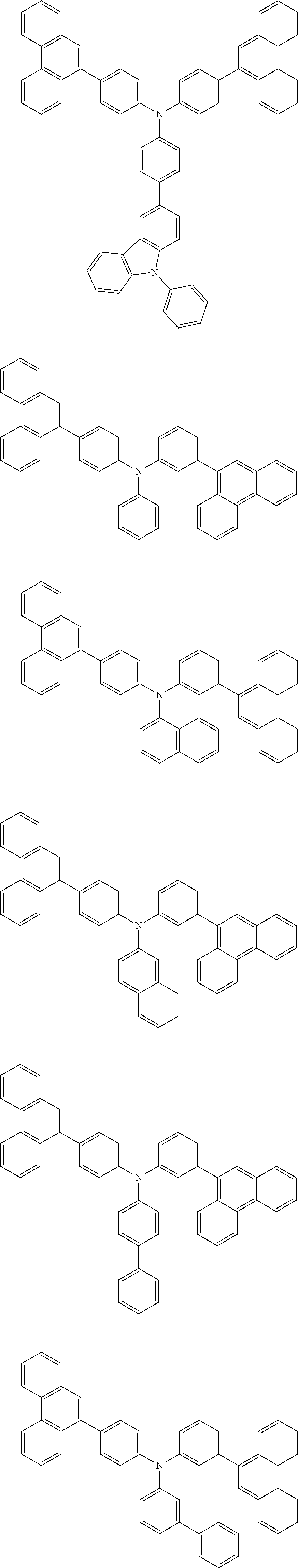 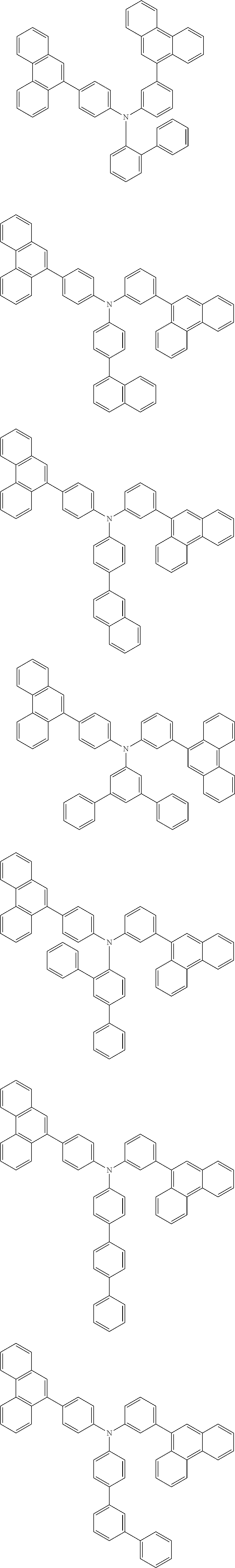 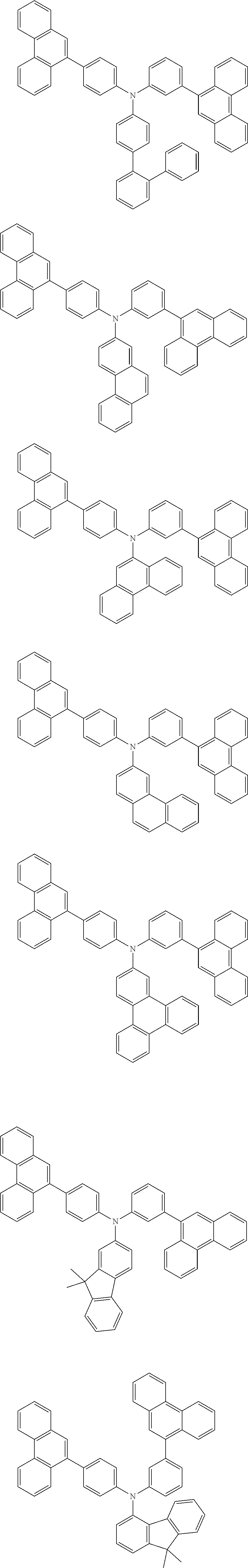 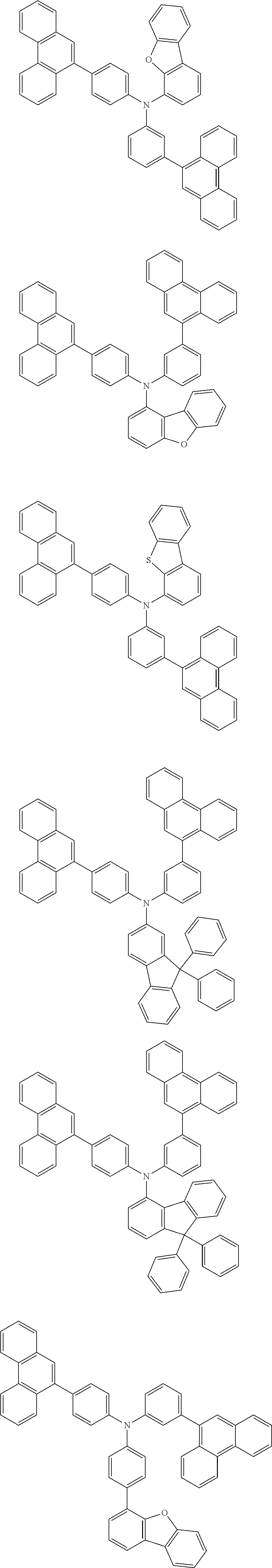 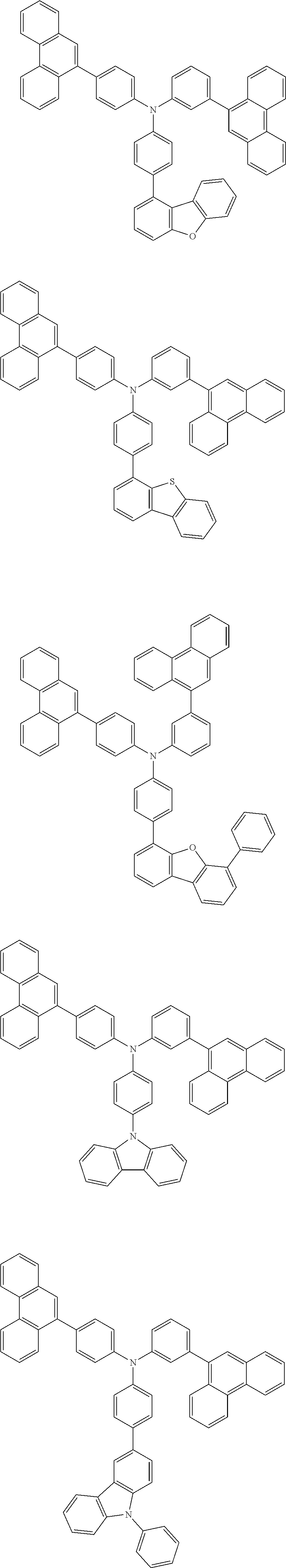 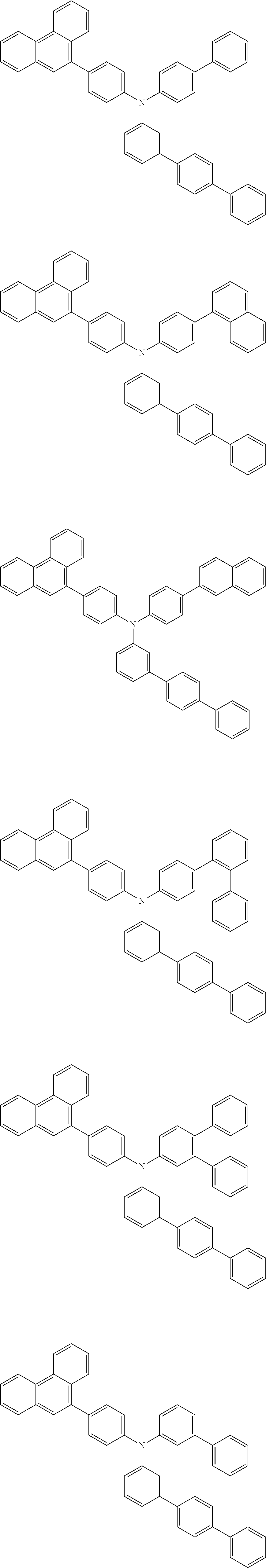 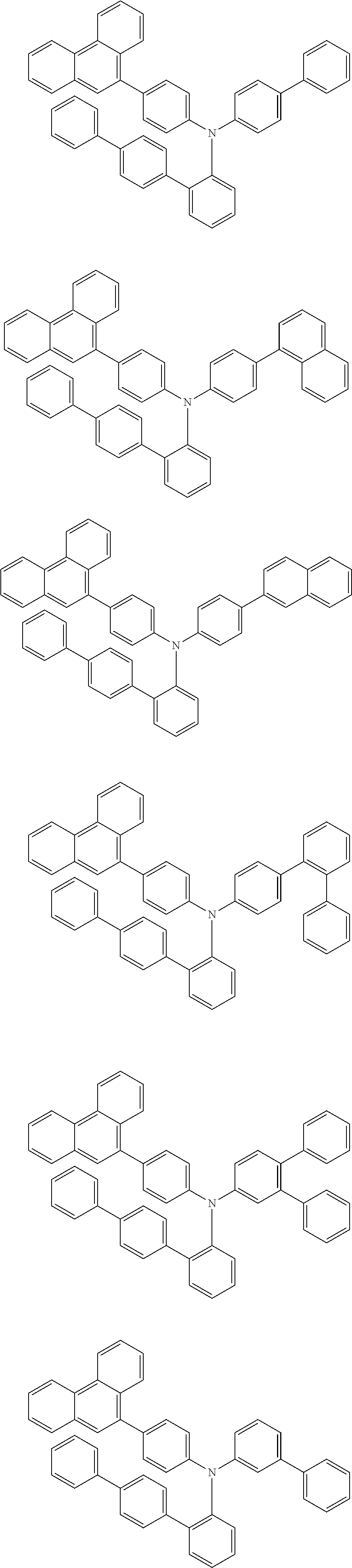 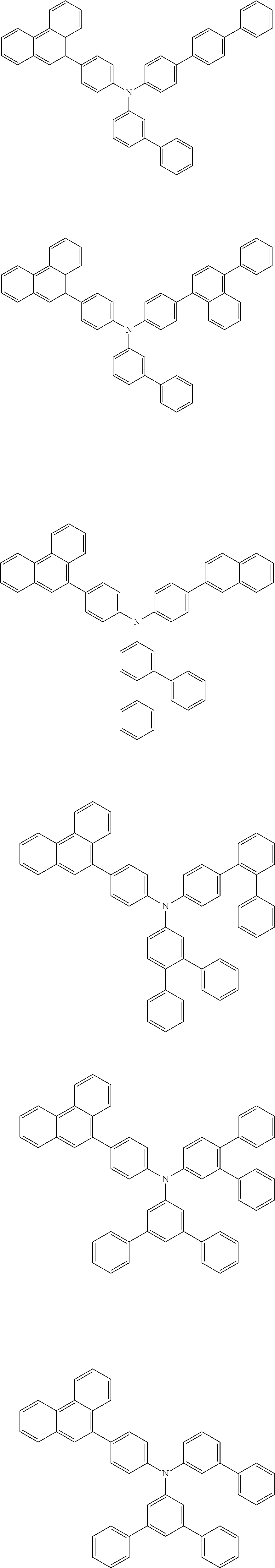 [chemical structure]
